(19) United States
(12) Patent Application Publication
(10) Pub. No.: US 2024/0132976 A1
(43) Pub. Date: Apr. 25, 2024

(54) METHODS OF STRATIFYING AND TREATING CORONAVIRUS INFECTION

(71) Applicants: Massachusetts Institute of Technology, Cambridge, MA (US); The Children's Medical Center Corporation, Boston, MA (US); University of Mississippi Medical Center, Jackson, MS (US)

(72) Inventors: Alexander K. Shalek, Cambridge, MA (US); Jose Ordovas-Montanes, Boston, MA (US); Carly Ziegler, Cambridge, MA (US); Sarah Glover, Jackson, MS (US); Bruce Horwitz, Boston, MA (US); Vincent Miao, Cambridge, MA (US); Anna Owings, Jackson, MS (US); Andrew Navia, Cambridge, MA (US); Ying Tang, Boston, MA (US); Joshua Bromley, Cambridge, MA (US)

(21) Appl. No.: 18/277,612

(22) PCT Filed: Feb. 18, 2022
(86) PCT No.: PCT/US2022/017082

§ 371 Date: Aug. 16, 2023

Related U.S. Application Data
(60) Provisional application No. 63/151,002, filed on Feb. 18, 2021.
(60) Provisional application No. 63/203,514, filed on Jul. 26, 2021.

(51) Int. Cl. C12Q 1/70; C12Q 1/6874; G01N 33/569

(52) U.S. Cl. CPC C12Q 1/701; C12Q 1/6874; G01N 33/56966; C12Q 2600/112; C12Q 2600/158

(57) ABSTRACT

The subject matter disclosed herein is generally directed to stratifying and treating coronavirus infections based on intrinsic immune states.

### CROSS-REFERENCE TO RELATED APPLICATIONS

This application claims the benefit of U.S. Provisional Application Nos. 63/151,002 filed Feb. 18, 2021 and 63/203,514 filed Jul. 26, 2021. The entire contents of the above-identified applications are hereby fully incorporated herein by reference.

### STATEMENT REGARDING FEDERALLY SPONSORED RESEARCH

This invention was made with government support under Grant Nos. GM007753, All 18672, and DK122532, awarded by the National Institutes of Health. The government has certain rights in the invention.

### REFERENCE TO AN ELECTRONIC SEQUENCE LISTING

The contents of the electronic sequence listing (“BROD-5375WP_ST25.txt”; Size is 8,000 bytes and it was created on Feb. 17, 2022) is herein incorporated by reference in its entirety.

### TECHNICAL FIELD

The subject matter disclosed herein is generally directed to determining whether a subject is at risk for severe respiratory disease from a coronavirus infection and treating the subject.

### BACKGROUND

The novel coronavirus clade SARS-CoV-2 emerged in late 2019 and has quickly led to one of the most devastating global pandemics in modern history. SARS-CoV-2 infection can cause severe respiratory COVID-19. However, many individuals present with isolated upper respiratory symptoms, suggesting potential to constrain viral pathology to the nasopharynx. Which cells SARS-CoV-2 primarily targets and how infection influences the respiratory epithelium remains incompletely understood. Similar to other successful respiratory viruses, high replication within the nasopharynx (Pan et al., 2020; Sanche et al., 2020) and viral shedding by asymptomatic or presymptomatic individuals contributes to high transmissibility (Fears et al., 2020; Meyerowitz et al., 2021) and rapid community spread (Arons et al., 2020; Sakurai et al., 2020; Wang et al., 2020c). COVID-19, the disease caused by SARS-CoV-2 infection, occurs in a fraction of those infected by the virus and carries profound morbidity and mortality. The clinical pictures of COVID-19 vary widely—from some individuals who experience few mild symptoms to some with prolonged and severe disease characterized by pneumonia, acute respiratory distress syndrome, and diverse systemic effects impacting various other tissues (Guan et al., 2020; Huang et al., 2020a). To facilitate effective preventative and therapeutic strategies for COVID-19, differentiating the host protective mechanisms that support rapid viral clearance and limit disease severity from those that drive severe and fatal outcomes is essential.

Rapid mobilization of the scientific community and a commitment to open data sharing early in the COVID-19 pandemic enabled researchers across the globe to study SARS-CoV-2 and build initial models of disease pathogenesis (Chan et al., 2020a; Wu et al., 2020; Zhou et al., 2020). By analogy to related human betacoronaviruses (Frieman and Baric, 2008), we currently understand viral tropism and disease progression to begin with SARS-CoV-2 entry through the mouth or nares where it initially replicates within epithelial cells of the human nasopharynx, generating an upper respiratory infection over several days (Harrison et al., 2020). A subset of patients develop symptoms of lower respiratory, where a combination of inflammatory immune responses and direct viral-mediated pathogenesis can lead to diffuse damage to distal airways, alveoli, and vasculature (Ackermann et al., 2020; Borczuk et al., 2020). However, the precise early targets for SARS-CoV-2 in the nasopharynx, the scope of potential host cells, and the variance in viral tropism across patients and disease courses have yet to be defined. A clearer understanding of viral tropism, how the airway epithelium responds to infection, and the relationship to disease outcome may critically inform future therapeutic or prophylactic strategies.

Citation or identification of any document in this application is not an admission that such a document is available as prior art to the present invention.

### SUMMARY

In one aspect, the present invention provides for a method of treating a barrier tissue infection in a subject in need thereof comprising: detecting one or more indicators of infection from a sample obtained from the subject, wherein the sample comprises one or more of epithelial, immune, stromal, and neuronal cells; comparing the indicators to control/healthy samples or disease reference values to determine whether the subject will progress to a risk group selected from: mild/moderate or severe; and administering one or more treatments if one or more indicators are present.

In certain embodiments, the barrier tissue infection is a respiratory barrier tissue infection. In certain embodiments, mild subjects are asymptomatic or symptomatic and not hospitalized, wherein moderate subjects are hospitalized and do not require oxygen by non-invasive ventilation or high flow, and wherein severe subjects are hospitalized and require oxygen by non-invasive ventilation, high flow, or intubation and mechanical ventilation. In certain embodiments, the infection is a viral infection. In certain embodiments, the viral infection is a coronavirus. In certain embodiments, the coronavirus is SARS-CoV2 or variant thereof. In certain embodiments, mild/moderate subjects have a WHO score of 1-5 and severe subjects have a WHO score of 6-8.

In certain example embodiments, one or more indicators of infection are selected from the group consisting of: decreased interferon-stimulated gene (ISG) induction; upregulation of one or more anti-viral factors or IFN-responsive genes; reduction of mature ciliated cell population or increased immature ciliated cell population; increased secretory cell population; increased deuterosomal cell population; increased ciliated cell population; increased goblet cell population; decreased expression in Type II interferon specific genes; increased expression in Type I interferon specific genes; increased MHC-I and MHC-II genes; increased developing ciliated cell populations; altered expression of one or more genes in a cell type selected from any of Tables 2-4; altered expression of one or more genes in a cell type selected from Table 5; increase expression of IFITM3 and IFI44L; increased expression of EIF2AK2; increased expression of TMPRSS4, TMPRSS2, CTSS, CTSD; upregulation of cholesterol and lipid biosynthesis; and increased abundance of low-density lipoprotein receptors LDLR and LRP8.

In certain embodiments, one or more interferon-stimulated genes are detected, wherein if the one or more interferon-stimulated genes are downregulated the subject is at risk for severe disease and if the one or more interferon-stimulated genes are upregulated the subject is not at risk for severe disease. In certain embodiments, the one or more interferon-stimulated genes are selected from the group consisting of STAT1, STAT2, IRF1, and IRF9.

In certain embodiments, the one or more indicators of infection are detected in infected host cells and compared to reference values in infected host cells from a risk group. In certain embodiments, one or more anti-viral factors or IFN-responsive genes are detected in virally-infected cells, wherein if the one or more anti-viral factors or IFN-responsive genes are downregulated or absent in virally-infected cells the subject is at risk for severe disease and if the one or more anti-viral factors or IFN-responsive genes are upregulated in virally-infected cells the subject is not at risk for severe disease. In certain embodiments, the one or more anti-viral factors or IFN-responsive genes are selected from the group consisting of EIF2AK2, STAT1 and STAT2.

In certain example embodiments, the secretory cells comprise one or both of: KRT13 KRT24 high Secretory Cells and Early Response Secretory Cells. In certain example embodiments, wherein the secretory cells express CXCL8. In certain example embodiments, the goblet cells comprise one or both of: AZGP1 high Goblet Cells and SCGB1A1 high Goblet Cells. In certain example embodiments, the ciliated cells comprise one or more upregulated genes selected from the group consisting of IFI27, IFIT1, IFI6, IFITM3, and GBP3. In certain example embodiments, one or both of the ciliated cells and the goblet cells comprise increased gene expression of one or more IFN gene selected from any of Tables 2-4. In certain example embodiments, ACE2 expression is upregulated compared to other epithelial cells among one or more of secretory cells, goblet cells, ciliated cells, developing ciliated cells, and deuterosomal cells. In certain example embodiments, the mature ciliated cells are BEST4 high cilia high ciliated cells. In certain example embodiments, the MHC-I and MHC-II genes comprise at least one or more of: HLA-A, HLA-C, HLA-F, HLA-E, HLA-DRB1, and HLA-DRA. In certain example embodiments, the upregulated cholesterol and lipid biosynthesis genes comprise at least one or more of: FDFT1, MVK, FDPS, ACAT2, and HMGCS1. In certain example embodiments, detecting one or more indicators is performed by using Simpson's index.

In certain example embodiments, a subject is determined to belong to the severe risk group if one or more of the following is detected in the sample: proinflammatory cytokines comprising at least one or more of: IL1B, TNF, CXCL8, CCL2, CCL3, CXCL9, CXCL10, and CXCL11; upregulation of alarmins comprising one or both of: S100A8 and S100A9; 14%-26% of all epithelial cells are secretory cells; elevated BPIFA1 high Secretory cells; elevated KRT13 KRT24 high secretory cells; macrophage population increase as compared to other immune cells; upregulated genes in ciliated cells comprising one or both of: IL5RA and NLRP1; no increase of at least one or more of: type I, type II, and type III interferon abundance; elevated stress response factors comprising at least one or more of: HSPA8, HSPA1A, and DUSP1; increased expression of one or more genes differentially expressed in COVID-19 WHO 6-8 according to Table 3 or Table 4; reduced or absent antiviral/interferon response; and reduced or absent mature ciliated cells. In certain example embodiments, the macrophage population comprises at least one or more of: ITGAX High Macrophages, FFAR High Macrophages, Inflammatory Macrophages, and Interferon Responsive Macrophages.

In certain example embodiments, a subject is determined to belong to the mild/moderate risk group if one or more of the following is detected in the sample: 4%-12% of all epithelial cells are Secretory Cells; 10%-20% of all epithelial cells comprise Interferon Responsive Ciliated Cells; upregulated ciliated cell genes comprising at least one or more of: IFI44L, STAT1, IFITM1, MX1, IFITM3, OAS1, OAS2, OAS3, STAT2, TAP1, HLA-C, ADAR, XAF1, IRF1, CTSS, and CTSB; increase in type I interferon abundance; high expression of interferon-responsive genes; decreased expression of one or more genes differentially expressed in COVID-19 WHO 6-8 according to Table 3 or Table 4; induction of type I interferon responses; and high abundance of IFI6 and IFI27.

In certain example embodiments, the interferon-responsive genes comprise at least one or more of: STAT1, MX1, HLA-B, and HLA-C. In certain example embodiments, the interferon response occurs in at least one or more of: MUC5AC high Goblet Cells, SCGB1A1 high Goblet Cells, Early Response Secretory Cells, Deuterosomal Cells, Interferon Responsive Ciliated Cells, and BEST4 high Cilia high Ciliated Cells.

In certain example embodiments, the treatment is administered according to determined risk group. In certain example embodiments, where the treatment involves administering a preventative or therapeutic intervention according to the determined risk group. In certain example embodiments, wherein if the subject is determined to be at risk for progression to the severe risk group the subject is administered a treatment comprising one or more treatments selected from the group consisting of: one or more antiviral; blood-derived immune-based therapy; one or more corticosteroid; one or more interferon; one or more interferon Type I agonists; one or more interleukin-1 inhibitors; one or more kinase inhibitors; one or TLR agonists; a glucocorticoid; and interleukin-6 inhibitor.

In certain example embodiments, if the subject is determined to be at risk for progression to either risk group the subject is administered a treatment comprising one or more of: one or more antiviral; one or more antibiotic; and one or more cholesterol biosynthesis inhibitor.

In certain example embodiments, the treatment comprises an antiviral. In certain example embodiments, the antiviral inhibits viral replication. In certain example embodiments, the antiviral is paxlovid, molnupiravir and remdesivir.

In certain example embodiments, the treatment is an immune-based therapy. In certain example embodiments, the immune-based therapy is a blood-derived product comprising at least one or more of: a convalescent plasma and an immunoglobin. In certain example embodiments, the immune-based therapy is an immunomodulator comprising at least one or more of: a corticosteroid, a glucocorticoid, an interferon, an interferon Type I agonist, an interleukin-1 inhibitor, an interleukin-6 inhibitor, a kinase inhibitor, and a TLR agonist. In certain example embodiments, the corticosteroid comprises at least one of: methylprednisolone, hydrocortisone, and dexamethasone. In certain example embodiments, the glucocorticoid comprises at least one of: cortisone, prednisone, prednisolone, methylprednisolone, dexamethasone, betamethasone, triamcinolone, Fludrocortisone acetate, deoxycorticosterone acetate, and hydrocortisone. In certain example embodiments, the interferon comprises at least one or more of: interferon beta-1b and interferon alpha-2b. In certain example embodiments, the interleukin-1 inhibitor comprises anakinra. In certain example embodiments, the interleukin-6 inhibitor comprises at least one or more of: anti-interleukin-6 receptor monoclonal antibodies and anti-interleukin-6 monoclonal antibody. In certain example embodiments, the anti-interleukin-6 receptor monoclonal antibody is tocilizumab. In certain example embodiments, the anti-interleukin-6 monoclonal antibody is siltuximab. In certain example embodiments, the kinase inhibitor comprises of at least one or more of Bruton's tyrosine kinase inhibitor and Janus kinase inhibitor. In certain example embodiments, the Bruton's tyrosine kinase inhibitor comprises at least one or more of: acalabrutinib, ibrutinib, and zanubrutinib. In certain example embodiments, the Janus kinase inhibitor comprises at least one or more of: baracitinib, ruxolitinib and tofacitinib. In certain example embodiments, the TLR agonist comprises at least one or more of: imiquimod, BCG, and MPL.

In certain example embodiments, the treatment comprises inhibiting cholesterol biosynthesis. In certain example embodiments, inhibiting cholesterol biosynthesis comprises administering HMG-CoA reductase inhibitors. In certain example embodiments, the HMG-CoA reductase inhibitor comprises at least one or more of: simvastatin atorvastatin, lovastatin, pravastatin, fluvastatin, rosuvastatin, pitavastatin. In certain example embodiments, the treatment comprises an antibiotic.

In certain example embodiments, the treatment comprises one or more agents capable of shifting epithelial cells to express an antiviral signature. In certain example embodiments, the treatment comprises one or more agents capable of suppressing a myeloid inflammatory response. In certain example embodiments, the treatment comprises an RNA-guided nuclease system. In certain example embodiments, the RNA-guided nuclease system is a CRISPR system. In certain example embodiments, the CRISPR system comprises a CRISPR-Cas base editing system, a prime editor system, or a CAST system.

In certain example embodiments, the treatment is administered before severe disease. In certain example embodiments, the infection is a viral infection. In certain example embodiments, the viral infection is a coronavirus. In certain example embodiments, coronavirus is SARS-CoV2 or variant thereof.

In certain example embodiments, the one or more cell types are detected using one or markers differentially expressed in the cell types. In certain example embodiments, the one or more cell types or one or more genes are detected by immunohistochemistry (IHC), fluorescence activated cell sorting (FACS), fluorescently bar-coded oligonucleotide probes, RNA FISH (fluorescent in situ hybridization), RNA-seq, or any combination thereof. In certain example embodiments, single cell expression is inferred from bulk RNA-seq. In certain example embodiments, expression is determined by single cell RNA-seq.

In another aspect, the present invention provides for a method of screening for agents capable of shifting epithelial cells from a SARS-CoV2 severe phenotype to a mild/moderate phenotype comprising: treating a sample comprising epithelial cells with a drug candidate; detecting modulation of any indicators of infection according to any of the preceding claims; and identifying the drug, wherein the one or more indicators shift towards a mild/moderate phenotype. In certain example embodiments, the sample comprises epithelial cells infected with SARS-CoV2. In certain example embodiments, the sample comprises epithelial cells expressing one or more SARS-CoV2 genes. In certain example embodiments, the sample is an organoid or tissue model. In certain example embodiments, the sample is an animal model. In certain example embodiments, cell types are detected using one or markers selected from Table 1.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1O—Cellular composition of nasopharyngeal swabs. FIG. 1A. Schematic of method for viable cryopreservation of nasopharyngeal swabs, cellular isolation, and scRNA-seq using the Seq-Well S\hat{ }3 platform (created with BioRender). FIG. 1B. UMAP of 32,588 single-cell transcriptomes from all participants, colored by cell type (following iterative Louvain clustering). FIG. 1C. UMAP as in B, colored by SARS-CoV-2 PCR status at time of swab. FIG. 1D. UMAP as in B, colored by peak level of respiratory support (WHO COVID-19 severity scale).

FIG. 1E. UMAP as in B, colored by participant. FIG. 1F. Violin plots of cluster marker genes (FDR <0.01) for coarse cell type annotations (as in B). FIG. 1G. Proportional abundance of coarse cell types by participant (ordered within each disease cohort by increasing Ciliated cell abundance). FIG. 1H. Proportional abundance of participants by coarse cell types. Shades of red: COVID-19. Shades of blue: Control. FIG. 1I. Expression of entry factors for SARS-CoV-2 and other common upper respiratory viruses. Dot size represents fraction of cell type (rows) expressing a given gene (columns). Dot hue represents scaled average expression. FIG. 1J. Proportion of Goblet Cells by sample. Statistical test above graph represents Kruskal-Wallis test results across all cohorts (following Bonferroni-correction). Statistical significance asterisks within box represent significant results from Dunn's post-hoc testing. * Bonferroni-corrected p-value <0.05, ** q<0.01, *** q<0.001. FIG. 1K. Proportion of Secretory Cells by sample. FIG. 1L. Proportion of Deuterosomal Cells by sample. FIG. 1M. Proportion of Developing Ciliated Cells by sample. FIG. 1N. Proportion of Ciliated Cells by sample. FIG. 1O. Simpson's Diversity index across epithelial cell types in COVID-19 vs. Control. Significance by Student's t-test.

FIGS. 2A-2R—Altered epithelial cell composition and recovery in the nasopharynx during COVID-19. FIG. 2A. UMAP of 28,948 epithelial cell types following re-clustering, colored by coarse cell types. Lines represent smoothed estimate of cellular differentiation trajectories (RNA velocity estimates via scVelo using intronic:exonic splice ratios).

FIG. 2B. UMAP as in A, colored by SARS-CoV-2 PCR status at time of swab. FIG. 2C. UMAP as in A, colored by peak level of respiratory support (WHO illness severity scale). FIG. 2D. UMAP as in A, colored by detailed cell annotations. FIG. 2E. Violin plots of cluster marker genes (FDR <0.01) for detailed epithelial cell type annotations (as in D). FIG. 2F. UMAP of 9,209 Basal, Goblet, and Secretory Cells, following sub-clustering and resolution of detailed cell annotations. FIG. 2G. UMAP of only Basal, Goblet, and Secretory Cells as in F, colored by SARS-CoV-2 PCR status at time of swab. FIG. 2H. UMAP of only Basal, Goblet, and Secretory Cells as in F, colored by inferred velocity pseudotime (darker blue shades: precursor cells, lighter yellow shades: more terminally differentiated cell types). FIG. 2I. Plot of gene expression by Basal, Goblet, and Secretory Cell velocity pseudotime for select genes. Points colored by detailed cell type annotations. FIG. 2J. UMAP of 13,913 Ciliated Cells, following sub-clustering and resolution of detailed cell annotations. FIG. 2K. UMAP of Ciliated Cells as in J, colored by SARS-CoV-2 PCR status at time of swab. FIG. 2L. UMAP of Ciliated Cells as in J, colored by inferred velocity pseudotime (darker blue shades: precursor cells, lighter yellow shades: more terminally differentiated cell types). FIG. 2M. Plot of gene expression by Ciliated Cell velocity pseudotime for select genes (all significantly correlated with velocity expression. Points colored by detailed cell type annotations. FIG. 2N. Proportion of Secretory Cell subtypes (detailed annotation) by sample, normalized to all epithelial cells. FIG. 2O. Proportion of Ciliated Cell subtypes (detailed annotation) by sample, normalized to all epithelial cells. FIG. 2P. UMAP of 13,210 epithelial cells (using UMAP embedding from A) from SARS-CoV-2 PCR negative participants (Control). Lines represent smoothed estimate of cellular differentiation trajectories (via RNA velocity) calculated using only cells from Control participants. FIG. 2Q. UMAP of 15,738 epithelial cells (using UMAP embedding from A) from SARS-CoV-2 PCR positive participants (COVID-19). Lines represent smoothed estimate of cellular differentiation trajectories (via RNA velocity) calculated using only cells from COVID-19 participants. Named cell types highlight those significantly altered between disease cohorts. FIG. 2R. UMAP of 32,588 cells from all participants, shaded by detailed cell type. Arrows represent smoothed estimate of cellular differentiation trajectories inferred by RNA Velocity.

FIGS. 3A-3J—Cell-type specific and shared transcriptional responses to SARS-CoV-2 infection. FIG. 3A. Abundance of significant differentially expressed (DE) genes by detailed cell type between Control WHO 0 vs. COVID-19 WHO 1-5 samples (left), Control WHO 0 and COVID-19 WHO 6-8 samples (middle), COVID-19 WHO 1-5 and COVID-19 WHO 6-8 samples (right). Restricted to genes with FDR-corrected p<0.001, log 2 fold change >0.25. ø=comparison not tested due to too few cells in one group. FIG. 3B. Top: Volcano plots of average log fold change vs. −log 10(FDR-adjusted p-value) for Ciliated cells (coarse annotation). Left: Control WHO 0 vs. COVID-19 WHO 1-5 (mild/moderate). Middle: Control WHO 0 vs. COVID-19 WHO 6-8 (severe). Right: COVID-19 WHO 1-5 (mild/moderate) vs. COVID-19 WHO 6-8 (severe). Horizontal red dashed line: FDR-adjusted p-value cutoff of 0.05 for significance. Bottom: gene set enrichment analysis plots across shared, type I interferon specific, and type II interferon specific stimulated genes. Genes are ranked by their average log fold change (FC) between each comparison. Black lines represent the ranked location of genes belonging to the annotated gene set. Bar height represents running enrichment score (NES: Normalized Enrichment Score). P-values following Bonferroni-correction: *** corrected p<0.001, ** p<0.01, * p<0.05. FIG. 3C. Heatmap of significantly DE genes between Interferon Responsive Ciliated Cells from different disease cohorts. FIG. 3D. Top: Volcano plots related to C. Average log fold change vs. −log 10(FDR-adjusted p-value) for Interferon Responsive Ciliated cells. Horizontal red dashed line: 0.05 cutoff for significance. Bottom: gene set enrichment analysis across shared, type I, and type II interferon stimulated genes. FIG. 3E. Heatmap of significantly DE genes between MUC5AC high Goblet Cells from different disease cohorts. FIG. 3F. Top: Volcano plots related to E. Average log fold change vs. -log 10(FDR-adjusted p-value) for MUC5AC high Goblet Cells. Horizontal red dashed line: 0.05 cutoff for significance. Bottom: gene set enrichment analysis across shared, type I, and type II interferon stimulated genes. FIG. 3G. Top: Dot plot of IFNGR1/2 and IFNAR1/2 gene expression by selected cell types. Bottom: Violin plots of gene module scores across selected cell types, split by Control WHO 0 (blue), COVID-19 WHO 1-5 (red), and COVID-19 WHO 6-8 (pink). Gene modules represent transcriptional responses of human basal cells from the nasal epithelium following in vitro treatment with IFNA or IFNG. Significance by Wilcoxon signed-rank test. P-values following Bonferroni-correction: * p<0.05, ** p<0.01, *** p<0.001. FIG. 3H. Common DE genes across detailed cell types. Left (red): genes upregulated in multiple cell types when comparing COVID-19 WHO 1-5 vs. Control WHO 0. Right (pink): genes upregulated in multiple cell types when comparing COVID-19 WHO 6-8 vs. Control WHO 0. FIG. 3I. Relative abundances of IgG autoantibodies for human type I, II, and III interferons via multiplexed human antigen microarray (see Methods). Blue circles: Control WHO 0, n=5; red circles: COVID-19 WHO 1-5, n=12; pink squares: COVID-19 WHO 6-8, n=8. Large pink squares: autoantibodies against 12 type I interferons from a single donor: COVID-19 Participant 27 (peak WHO severity score: 8, swab WHO severity score: 5). FIG. 3J. Average expression of STAT1, STAT2, IRF1, and IRF9 among ciliated cells by participant. For each gene: left: participants separated by disease group, determined by participants' peak WHO severity score. Statistical testing by Kruskal-Wallis test across disease groups (** p=0.0018) with Dunn's post hoc testing: * p<0.05, ** p<0.01, *** p<0.001. Right: participants in COVID-19 WHO 6-8 group, separated by level of severity at time of nasal swab. Statistical testing by Wilcoxon signed-rank test, n.s. non-significant, p >0.05.

FIGS. 4A-4H—Co-detection of human and SARS-CoV-2 RNA. FIG. 4A. Metatranscriptomic classification of all single-cell RNA-seq reads using Kraken2. Results shown from selected respiratory viruses. Only results with greater than 5 reads are shown. FIG. 4B. Normalized abundance of SARS-CoV-2 aligning UMI from all single-cell RNA-seq reads (including those derived from ambient/low-quality cell barcodes). P<0.0001 by Kruskal-Wallis test. Pairwise comparisons using Dunn's post-hoc testing. ** p<0.01, *** p<0.001. FIG. 4C. Proportional abundance of Secretory cells (all) vs. total SARS-CoV-2 UMI (normalized to M total UMI). FIG. 4D. Proportional abundance of FOXJ1 high Ciliated cells vs. total SARS-CoV-2 UMI (normalized to M total UMI). FIG. 4E. SARS-CoV-2 UMI per high-quality cell barcode. Results following correction for ambient viral reads. FIG. 4F. Schematic for SARS-CoV-2 genome and subgenomic RNA species. FIG. 4G. Schematic for SARS-CoV-2 genomic features annotated in the custom reference gtf. FIG. 4H. Heatmap of SARS-CoV-2 genes expression among SARS-CoV-2 RNA+ single cells (following correction for ambient viral reads). Top color bar indicates disease and severity cohort (red: COVID-19 WHO 1-5, pink: COVID-19 WHO 6-8, black: COVID-19 convalescent, blue: Control WHO 0). Top heatmap: SARS-CoV-2 genes and regions organized from 5′ to 3′. Bottom heatmap: alignment to 70-mer regions directly surrounding viral transcription regulatory sequence (TRS) sites, suggestive of spliced RNA species (joining of the leader to body regions) vs. unspliced RNA species (alignment across TRS).

FIGS. 5A-5E—Cellular targets of SARS-CoV-2 in the nasopharynx. FIG. 5A. Summary schematic of top SARS-CoV-2 RNA+ cells. (created with BioRender). FIG. 5B. SARS-CoV-2 RNA+ cell abundance (top) and percent (bottom) per participant. Results following correction for ambient viral reads. FIG. 5C. Abundance of SARS-CoV-2 RNA+ cells by detailed cell type, bars colored by participant. Results following correction for ambient viral reads. FIG. 5D. Dot plot of SARS-CoV-2 RNA presence by sample (columns) and detailed cell types (rows). Dot size reflects fraction of a given participant and cell type containing SARS-CoV-2 RNA (following viral ambient correction). Dot color reflects fraction of aligned reads corresponding to the SARS-CoV-2 positive strand (yellow) vs. negative strand (black). Dot plot across columns: alignment of viral reads by participant, separated by RNA species type. Dot plot across rows: alignment of viral reads by detailed cell type, separated by RNA species type. FIG. 5E. Percent ACE2+ cells vs. percent SARS-CoV-2 RNA+ cells by coarse cell type (left) and detailed cell type (right).

FIGS. 6A-6F—Intrinsic and bystander responses to SARS-CoV-2 infection. FIG. 6A. Violin plot of selected genes upregulated in SARS-CoV-2 RNA+ cells in at least 3 individual cell type comparisons. Dark red: SARS-CoV-2 RNA+ cells, red: bystander cells from COVID-19 participants, blue: cells from Control participants. From left to right the scale is log(1+UMI per 10K) FIG. 6B. Enriched gene ontologies among genes consistently up- or down-regulated among SARS-CoV-2 RNA+ cells across cell types. FIG. 6C. Heatmap of genes consistently higher in SARS-CoV-2 RNA+ cells across multiple cell types. Colors represent log fold changes between SARS-CoV-2 RNA+ cells and bystander cells (SARS-CoV-2 RNA− cells, from COVID-19 infected donors) by cell type. Restricted to cell types with at least 5 SARS-CoV-2 RNA+ cells. Yellow: upregulated among SARS-CoV-2 RNA+ cells, blue: upregulated among bystander cells. FIG. 6D. Heatmap of genes consistently higher in bystander cells across multiple cell types. FIG. 6E. Top: Violin plots of SARS-CoV-2 aligning reads among SARS-CoV-2 RNA+ cells. Statistical significance by Wilcoxon rank sum test. Bottom: select differentially expressed genes between SARS-CoV-2 RNA+ cells from participants with mild/moderate COVID-19 (red) vs. severe COVID-19 (pink). Statistical significance by likelihood ratio test assuming an underlying negative binomial distribution. * ** FDR-corrected p<0.001, ** p<0.01, * p<0.05. FIG. 6F Percent ACE2+ cells vs. percent SARS-CoV-2 RNA+ cells by detailed cell type. Left: cells from participants with mild/moderate COVID-19. Right: cells from participants with severe COVID-19. Point size reflects average type I interferon specific module score among SARS-CoV-2 RNA+ cells.

FIGS. 7A-7N—Participant cohort and cellular composition of nasopharyngeal swabs. FIG. 7A. Cohort composition and participant demographics. FIG. 7B. IgM and IgG titers among Control WHO 0 and COVID-19 participants. FIG. 7C. Detailed schematic of sample preparation and cell processing from nasal swabs (created with BioRender). FIG. 7D. Single cell quality metrics by cohort (after filtering for low-quality cells). FIG. 7E. Single cell quality metrics by participant (after filtering for low quality cells). FIG. 7F. Quality metrics for matched fresh vs. frozen nasal swabs from two participants (P1 and P2). FIG. 7G. UMAP of cell types from P1. FIG. 7H. UMAP of cell types from P2. FIG. 7I. Percent composition of each cell type by fresh (grey circles) or frozen (black squares) processing. FIG. 7J. UMAP from P1 as in G, colored by fresh (grey) vs. frozen (black). FIG. 7K. UMAP from P2 as in H, colored by fresh (grey) vs. frozen (black). FIG. 7L. Comparison of WHO severity at swab and peak. FIG. 7M. Comparison of WHO severity at swab and peak. FIG. 7N. Number of high-quality cells/array recovered for single-cell RNA-seq by disease group. Statistical testing by Kruskal-Wallis test (p=0.37) with Dunn's post hoc testing, all p >0.05.

FIGS. 8A-8G—COVID-19-induced changes to epithelial diversity and differentiation. FIG. 8A. Proportional abundance of detailed epithelial cell types by participant. FIG. 8B. Expression of entry factors for SARS-CoV-2 and other common upper respiratory viruses among detailed epithelial cell types. Dot size represents fraction of cell type (rows) expressing a given gene (columns). Dot hue represents average expression. FIG. 8C. Plot of gene expression by epithelial cell velocity pseudotime. Select genes significantly associated with ciliated cell pseudotime. Points colored by coarse cell type annotations. Top: alignment to unspliced (intronic) regions. Bottom: alignment to spliced (exonic) regions. FIG. 8D. Proportion of Goblet Cell subtypes (detailed annotation) by sample, normalized to all epithelial cells. Statistical test above graph represents Kruskal-Wallis test results across all cohorts (following Bonferroni-correction). FIG. 8E. Flow cytometry and gating scheme of immune cells from a fresh nasopharyngeal (NP) swab. Representative healthy participant. Bottom right: quantification of cellular proportions. FIG. 8F. Flow cytometry and gating scheme of epithelial cells from an NP swab. Representative data from a participant with severe COVID-19. FIG. 8G. Secretory cell proportion of live, CD45− cells from NP swabs. Healthy donors (Control WHO 0): n=7. Severe COVID-19 (COVID-19 WHO 6-8): n=7. Secretory cells identified as Live, CD45⁻ATubulin-CD271⁻CD49f⁻CD66c⁺ cells. Statistical testing: Wilcoxon signed-rank test: ** p=0.0047.

FIGS. 9A-9L—COVID-19-induced changes to nasopharynx-resident immune cells. FIG. 9A. UMAP of 3,640 immune cells following re-clustering, colored by coarse cell types. FIG. 9B. UMAP as in A, colored by detailed cell annotations. FIG. 9C. UMAP as in A, colored by level of respiratory support (WHO illness severity scale). FIG. 9D. UMAP as in A, colored by SARS-CoV-2 PCR status at time of swab. FIG. 9E. UMAP as in A, colored by participant. FIG. 9F. Violin plots of cluster marker genes (FDR <0.01) for detailed immune cell type annotations (as in B). FIG. 9G. Proportional abundance of detailed immune cell types by participant. FIG. 9H. Proportion of immune cell subtypes by sample and cohort, normalized to all immune cells. Statistical test above graph represents Kruskal-Wallis test results across all cohorts (following Bonferroni-correction). FIG. 9I. Heatmap of significantly DE genes between Macrophages (all, coarse annotation) from different disease cohorts. FIG. 9J. Heatmap of significantly DE genes between T Cells (all, coarse annotation) from different disease cohorts. FIG. 9K. Top: Dot plot of IFNGR1/2 and IFNAR1/2 gene expression among all detailed immune subtypes. Bottom: Violin plots of gene module scores, split by Control WHO 0 (blue), COVID-19 WHO 1-5 (red), and COVID-19 WHO 6-8 (pink). Gene modules represent transcriptional responses of human basal cells from the nasal epithelium following in vitro treatment with IFNA or IFNG. Significance by Wilcoxon signed-rank test. P-values following Bonferroni-correction: * p<0.05, ** p<0.01, *** p<0.001. FIG. 9L. Proportion of interferon responsive macrophages vs. proportion of interferon responsive cytotoxic CD8 T cells per sample, normalized to total immune cells. Including all samples, Control and COVID-19 groups.

FIGS. 10A-10H—Cell-type specific and shared transcriptional responses to SARS-CoV-2 infection. FIG. 10A. Abundance of significant differentially expressed genes by coarse cell type between Control WHO 0 and COVID-19 WHO 1-5 samples (left), Control WHO 0 and COVID-19 WHO 6-8 samples (middle) and COVID-19 WHO 1-5 vs. COVID-19 WHO 6-8 samples (right). FDR-corrected p<0.001, log 2 fold change >0.25. FIG. 10B. Heatmap of significantly DE genes between Ciliated Cells (all, coarse annotation) from different disease cohorts. FIG. 10C. Venn diagram of significantly upregulated genes among Ciliated Cells between COVID-19 WHO 1-5 vs Control WHO 0 (red) and COVID-19 WHO 6-8 vs. Control WHO 0 (pink). Asterisk: genes impacted by steroid treatment within each cohort. FIG. 10D. Interferon gene module scores across all detailed epithelial cell types, split by Control WHO 0 (blue), COVID-19 WHO 1-5 (red), and COVID-19 WHO 6-8 (pink). Gene modules represent transcriptional responses of human basal cells from the nasal epithelium following in vitro treatment with IFNA or IFNG. FIG. 10E. Dot plot of ACE2 expression across select coarse and detailed epithelial cell types and subsets. FIG. 10F. Dot plot of interferon and cytokine expression among detailed epithelial and immune cell types. FIG. 10G. Violin plots of select genes upregulated among ciliated cells in COVID-19 WHO 1-5 participants compared to Control WHO 0 (PARP14, ISG1S) and in COVID-19 WHO 6-8 participants compared to Control WHO 0 (FKBP5). Cells separated by participant treatment with corticosteroids. *** FDR-corrected p<0.001. FIG. 10H. Dot plot of type I and type III interferons among ciliated, goblet, and squamous cells. Left: healthy vs. influenza A/B virus infected participants from Cao et al., 2020. Right: Control WHO 0 vs. COVID-19 WHO 1-5, vs. COVID-19 WHO 6-8 participants. Datasets processed and scaled identically.

FIGS. 11A-11J—Detection of SARS-CoV-2 RNA from single-cell RNA-seq data. FIG. 11A. Metatranscriptomic classification of all single-cell RNA-seq reads using Kraken2: reads per sample annotated as unclassified. FIG. 11B. Metatranscriptomic classification of all single-cell RNA-seq reads using Kraken2: reads per sample annotated as Homo sapiens. FIG. 11C. Metatranscriptomic classification of all single-cell RNA-seq reads using Kraken2: reads per sample annotated as SARS-related coronaviruses. FIG. 11D. Total recovered cells per sample vs. normalized abundance of SARS-CoV-2 aligning UMI from all single-cell RNA-seq reads (including those derived from ambient/low-quality cell barcodes). FIG. 11E. Normalized abundance of SARS-CoV-2 aligning UMI from all single-cell RNA-seq reads across all COVID-19 participants. Dashed line represents partition between “Viral High” vs “Viral Low” samples. FIG. 11F. Proportional abundance of selected cell types according to total SARS-CoV-2 abundance among COVID-19 samples. Statistical test above graph represents Kruskal-Wallis test statistic across all cohorts. Statistical significance asterisks within box represent significant results from Dunn's post-hoc testing. Bonferroni-corrected p-value: * p<0.05, ** p<0.01, * ** p<0.001. FIG. 11G. Abundance of SARS-CoV-2 aligning UMI/cell by participant prior to (top) and following (bottom) ambient viral RNA correction. FIG. 11H. Quality metrics among 415 SARS-CoV-2 RNA+ cells (associated with high-quality cell barcodes and following ambient viral RNA correction). Left: abundance of SARS-CoV-2 aligning UMI vs. percent of all aligned reads (per cell barcode) aligning to SARS-CoV-2. Middle: abundance of human (GRCh38)-aligning UMI vs. abundance of SARS-CoV-2 aligning UMI. Right: abundance of human (GRCh38) aligning UMI vs. percent of all aligned reads (per cell barcode) aligning to human genes. FIG. 11I. Normalized abundance of SARS-CoV-2 aligning UMI vs. anti-SARS-CoV-2 IgM (left) or IgG titers (right). Plasma samples taken on same day of nasopharyngeal swab. Subset of Control WHO 0 (blue circles, n=13) and COVID-19 (red circles, mild/moderate: n=8; pink squares, severe: n=15) participants. Dashed lines: lower limit of detection: 100; upper limit of detection: 100,000; positive threshold: 5,000. Pearson's correlation of COVID-19 samples: IgM: r=−0.59, ** p=0.0028; IgG: r=−0.60, ** p=0.0025. FIG. 11J. Percent SARS-CoV-2 RNA+ cells (associated with high-quality cell barcodes and following ambient viral RNA correction) per donor, separated by disease group. Statistical test above graph represents Kruskal-Wallis test statistic across all groups. Statistical significance asterisks within box represent significant results from Dunn's post-hoc testing. * p<0.05, ** p<0.01.

FIGS. 12A-12H—SARS-CoV-2 RNA species and cell types containing viral reads. FIG. 12A. Schematic of method to distinguish unspliced from spliced SARS-CoV-2 RNA species by searching for reads which align across a spliced or genomic Transcription Regulatory Sequence (TRS, 6mer). FIG. 12B. Abundance of SARS-CoV-2 aligning UMI/Cell per detailed cell type (following ambient viral RNA correction), split by UMI aligning to the viral positive strand, negative strand, 70-mer region across an unspliced TRS, and 70-mer region across a spliced TRS. FIG. 12C. Abundance of SARS-CoV-2 aligning UMI/Cell per participant (following ambient viral RNA correction), split by UMI aligning to the viral positive strand, negative strand, 70-mer region across an unspliced TRS, and 70-mer region across a spliced TRS. FIG. 12D. Dot plot of SARS-CoV-2 unspliced TRS aligning UMI by participant (columns) and detailed cell type (rows). FIG. 12E. Dot plot of SARS-CoV-2 spliced TRS aligning UMI by participant (columns) and detailed cell type (rows). FIG. 12F. Percent ACE2+ cells vs. percent SARS-CoV-2 RNA+ (after ambient correction) by detailed cell type. Including only cells from COVID-19 participants. Statistical testing using spearman's correlation. FIG. 12G. Abundance of SARS-CoV-2 negative strand aligning reads by coarse epithelial cell types. FIG. 12H. Abundance of SARS-CoV-2 negative strand aligning reads by detailed ciliated cell types.

FIGS. 13A-13C—Intrinsic and bystander responses to SARS-CoV-2 infection. FIG. 13A. Violin plots of select genes upregulated in SARS-CoV-2 RNA+ Cells when compared to matched bystanders. Plotting only SARS-CoV-2 RNA+ Cells from COVID-19 WHO 1-5 participants (red) and COVID-19 WHO 6-8 participants (pink). Top row: SARS-CoV-2 RNA expression by alignment type. FIG. 13B. Heatmaps of log fold changes between SARS-CoV-2 RNA+ cells and bystander cells by cell types. Gene sets derived from four CRISPR screens for important host factors in the SARS-CoV-2 viral life cycle. Restricted to cell types with at least 5 SARS-CoV-2 RNA+ cells. Yellow: upregulated among SARS-CoV-2 RNA+ cells, blue: upregulated among bystander cells. FIG. 13C. Heatmap of Spearman's correlation between 73 clinical parameters, demographic data, or results from scRNA-seq. Includes individuals from healthy (Control WHO 0), COVID-19 mild/moderate (COVID-19 WHO 1-5) and COVID-19 severe (COVID-19 WHO 6-8) groups. Colored squares represent statistically significant associations by permutation test (p<0.01; red: positive Spearman's rho; blue: negative Spearman's rho).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to a manuscript entitled “Impaired local intrinsic immunity to SARS-CoV-2 infection in severe COVID-19,” uploaded to Biorxiv on Feb. 18, 2021 and having the following authors: Carly G. K. Ziegler, Vincent N. Miao, Anna H. Owings, Andrew W. Navia, Ying Tang, Joshua D. Bromley, Peter Lotfy, Meredith Sloan, Hannah Laird, Haley B. Williams, Micayla George, Riley S. Drake, Taylor Christian, Adam Parker1, Campbell B. Sindel, Molly W. Burger, Yilianys Pride, Mohammad Hasan, George E. Abraham III, Michal Senitko, Tanya O. Robinson, Alex K. Shalek, Sarah C. Glover, Bruce H. Horwitz, Jose Ordovas-Montanes. Reference is also made to U.S. patent application Ser. No. 16/631,898, published as US20200158716A1 and claiming priority to PCT/US2018/042557. Reference is also made to Ziegler C G K, Miao V N, Owings A H, et al. Impaired local intrinsic immunity to SARS-CoV-2 infection in severe COVID-19. Cell. 2021; 184(18):4713-4733.e22. doi:10.1016/j.cell.2021.07.023. All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods of determining whether a subject is at risk for severe respiratory disease from a coronavirus infection and treating subjects at risk prophylactically or subjects suffering from severe respiratory disease. SARS-CoV-2, the virus that causes COVID-19, relies on efficient replication within cells of the human upper airways for infection and transmission. In some individuals, the virus accesses lower respiratory tissues, causing pneumonia, acute respiratory distress syndrome, and systemic effects which lead to profound morbidity and mortality. Despite major advances in understanding peripheral correlates of immunity during COVID-19, how SARS-CoV-2 impacts its primary target tissue, the human nasopharynx, remains unclear. Here, Applicants present a cohort of over 60 samples from healthy individuals and participants with COVID-19, representing a wide spectrum of disease states from ambulatory to critically ill. Using standard nasopharyngeal swabs, Applicants collected viable cells and performed single-cell RNA-seq, simultaneously profiling both host and viral RNA. Applicants performed scRNA-seq on nasopharyngeal swabs from 58 healthy and COVID-19 participants. Applicants find that following infection with SARS-CoV-2 the upper respiratory epithelium undergoes massive expansion and diversification of secretory cells and preferential loss of mature ciliated cells. During COVID-19, Applicants observe expansion of secretory, loss of ciliated, and epithelial cell repopulation via deuterosomal expansion. Active repopulation of lost ciliated cells appears to occur through secretory cell transdifferentiation via deuterosomal cell intermediates. Epithelial cells from participants with mild/moderate COVID-19 showed extensive induction of genes associated with anti-viral and type I interferon responses. In contrast, cells from participants with severe lower respiratory symptoms appear globally stunted in their anti-viral capacity, despite substantially higher local inflammatory myeloid populations and equivalent nasal viral loads: suggesting an essential role for intrinsic, local epithelial immunity in curbing and constraining viral infection. In mild/moderate COVID-19, epithelial cells express anti-viral/interferon-responsive genes, while cells in severe COVID-19 have muted anti-viral responses despite equivalent viral loads. Through a custom computational pipeline, Applicants characterized cell-associated SARS-CoV-2 RNA and identified rare cells with RNA intermediates strongly suggestive of active replication. Among SARS-CoV-2 RNA+ host cells, Applicants found remarkable diversity and heterogeneity both within and across individuals, including developing/immature and interferon-responsive ciliated cells, KRT13+ “hillock”-like cells, and unique subsets of secretory, goblet, and squamous cells. SARS-CoV-2 RNA+ host-target cells are highly heterogenous, including developing ciliated, interferon-responsive ciliated, AZGP1^{thigh} goblet, and KRT13+ “hillock”-like cells, and Applicants identify genes associated with susceptibility, resistance, or infection response. Finally, among SARS-CoV-2 RNA+ cells, Applicants detected genes that were enriched compared to uninfected bystanders, suggesting involvement in either the cell-intrinsic response or susceptibility to infection. These included anti-viral genes (e.g., MXJ, IFITM3, EIF2AK2), proteases (e.g., CTSL, TMPRSS2), and pathways involved in cholesterol biosynthesis. Together, this work defines the protective and detrimental host responses to SARS-CoV-2, determines the direct viral targets of infection, and suggests that failed cell-intrinsic anti-viral epithelial immunity in the nasal mucosa underlies the progression to severe COVID-19. The study defines protective and detrimental responses to SARS-CoV-2, the direct viral targets of infection, and suggests that failed nasal epithelial anti-viral immunity may underlie and precede severe COVID-19.

The present invention stratifies subjects based on their risk of developing severe respiratory disease or if the subject is predicted to have mild/moderate disease. The present invention also provides for predicting the risk of developing severe respiratory disease in subjects who initially present as asymptomatic or as mild/moderate disease. As used herein, the terms “severe” refers to a subject having intubation and mechanical ventilation, ventilation with additional organ support, or death. As used herein, the terms “mild” refers to a subject having no limitation of activities, limitation of activities, hospitalized and no oxygen therapy, oxygen by mask or nasal prongs, non-invasive ventilation or high-flow oxygen. As used herein, the terms “moderate” refers to a subject having no limitation of activities, limitation of activities, hospitalized and no oxygen therapy, oxygen by mask or nasal prongs, non-invasive ventilation or high-flow oxygen.

| Patient State | Descriptor | Score |
|---|---|---|
| Uninfected | No clinical or virological | 0 |
| | evidence of infection | |
| Ambulatory | No limitation of activities | 1 |
| | Limitation of activities | 2 |
| Hospitalized | Hospitalized, no oxygen | |
| Mild disease | therapy | |
| | Oxygen by mask or nasal | 4 |
| | prongs | |
| Hospitalized | Non-invasive ventilation or | 5 |
| Severe Disease | high-flow oxygen | |
| | Intubation and mechanical | 6 |
| | ventilation | |
| | Ventilation + additional organ | 7 |
| | support - pressors, RRT, | |
| | ECMO | |
| Dead | Death | 8 |

The present invention provides for cell subsets and cell states identified using single cell RNA sequencing of nasopharyngeal swabs from a large patient cohort of SARS-CoV-2 positive subjects. As used herein cell subsets refers to a cell that can be distinguished by a parent cell type, but expresses a specific gene signature or cell state that can further distinguish the cell from other cells of the parent cell type. As used herein, cell subsets are also referred to by a cluster (i.e., the different cell subsets cluster together). In certain embodiments, shifts in cell types or subsets of a cell type are used to predict a disease state and for selecting a treatment. In certain embodiments, shifts in cell states in cell types or subsets of a cell type and are used to predict a disease state and for selecting a treatment. As used herein, cell state refers to the expression of genes in specific cell subsets. As used herein, gene expression is not limited to mRNA expression and may also include proteins. In certain embodiments, the cell subset frequency and/or cell states can be detected for screening novel therapeutics. The present invention provides for subsets of epithelial cell types and immune cells. In certain embodiments, intrinsic immune responses are differentially induced in different patient populations (e.g., severe, mild or moderate). In certain embodiments, intrinsic immune states or conditions are monitored or detected during treatment. In certain embodiments, the frequency of the cell subsets are shifted in disease states. Disease states may include disease severity or response to any treatment in the standard of care for the disease.

In certain embodiments, one or more cell subsets associated with a disease state or risk group is detected or shifted to a treat a subject in need thereof. In certain embodiments, the cell subsets can be identified using one or more marker genes specific for the subset. In certain embodiments, the cell subsets that are shifted include KRT13 KRT24 high Secretory Cells, Early Response Secretory Cells, CXCL8 Secretory Cells, AZGP1 high Goblet Cells, SCGB1A1 high Goblet Cells, IFI27; IFIT1; IFI6; IFITM3; and GBP3 ciliated cells, any IFN gene ciliated cells, any IFN goblet cells, ACE2 epithelial cells, ACE2 secretory cells, ACE2 goblet cells, ACE2 ciliated cells, ACE2 developing ciliated cells, ACE2 deuterosomal cells, BEST4 high cilia high ciliated cells. Applicants have identified specific markers for each cell subset using single cell RNA sequencing (scRNA-seq) (see, e.g., Table 1). In certain embodiments, 1, 2, 3, 4, 5, 6, 7, 8, 9 or 10 or more genes are detected. In certain embodiments, detecting 2 or more of the subset markers increases the probability of detecting a cell subset.

In certain embodiments, specific cell types or cell subtypes differentially express genes based on the disease state or risk of the disease state. Applicants have identified specific differentially expressed genes in specific cell types using single cell RNA sequencing (scRNA-seq). In particular, Applicants identified differentially expressed genes in specific cell types between subjects having different severity of disease (see, e.g., Tables 2-4). In certain embodiments, genes differentially expressed between WHO score 0 (healthy) and WHO score 1-5 (mild/moderate) (Table 2) indicate genes that are expressed in subjects to reduce virus severity. In certain embodiments, a treatment would increase expression of one or more of these genes. In certain embodiments, detection of one or more of these genes indicates that the subject does not have a severe disease or risk of severe disease. In certain embodiments, genes differentially expressed between WHO score 0 (healthy) and WHO score 6-8 (severe) (Table 3) indicate genes that are expressed in subjects to reduce virus severity and/or generate an intrinsic immune response that leads to severe disease. In certain embodiments, a treatment would decrease expression of one or more of these genes. In certain embodiments, detection of one or more of these genes indicates that the subject has a severe disease or risk of severe disease. In certain embodiments, genes differentially expressed between WHO score 1-5 (mild/moderate) and WHO score 6-8 (severe) (Table 4) indicate genes that are expressed in subjects generate an intrinsic immune response that leads to severe disease. In certain embodiments, a treatment would decrease expression of one or more of these genes. In certain embodiments, detection of one or more of these genes indicates that the subject has a severe disease or risk of severe disease.

In certain embodiments, a cell state associated with a disease state or risk group is detected or shifted to a treat a subject in need thereof. In certain embodiments, the cell states can be identified using one or more differentially expressed genes in specific cell types between risk groups. In certain embodiments, 1, 2, 3, 4, 5, 6, 7, 8, 9 or 10 or more genes are detected. In certain embodiments, 10, 20, 30, 40, 50, 60, 70, 80, 90 or more than 100 genes are detected. In certain embodiments, detecting 2 or more of the differentially expressed genes increases the probability of detecting a subject having a cell state indicative of a specific intrinsic immune state and risk of severe disease.

In certain embodiments, the methods of the present invention use control values for the frequency of subsets and cell states. For example, the present nasal swab single cell atlas provides for the frequency of cell subsets and cell states for each of healthy WHO score 0 and COVID WHO score 1-8 subjects. Cells such as disclosed herein may in the context of the present specification be said to “comprise the expression” or conversely to “not express” one or more markers, such as one or more genes or gene products; or be described as “positive” or conversely as “negative” for one or more markers, such as one or more genes or gene products; or be said to “comprise” a defined “gene or gene product signature”.

Such terms are commonplace and well-understood by the skilled person when characterizing cell phenotypes. By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells. In regard to frequency, a cell subset may be present or not present. In certain embodiments, a cell subset may be 5, 10, 20, 30, 40, 50, 60, 70, 80 or 90% more frequent in a parent cell population as compared to a control level.

In certain embodiments, the cell state is a gene program comprising one or more up and down regulated genes. Clusters (subsets) and gene programs as described herein can also be described as a metagene. As used herein a “metagene” refers to a pattern or aggregate of gene expression and not an actual gene. Each metagene may represent a collection or aggregate of genes behaving in a functionally correlated fashion within the genome. The metagene can be increased if the pattern is increased. As used herein the term “gene program” or “program” can be used interchangeably with “cell state”, “biological program”, “expression program”, “transcriptional program”, “expression profile”, “signature”, “gene signature” or “expression program” and may refer to a set of genes that share a role in a biological function (e.g., an antiviral program, inflammatory program, cell differentiation program, proliferation program). Biological programs can include a pattern of gene expression that result in a corresponding physiological event or phenotypic trait (e.g., inflammation). Biological programs can include up to several hundred genes that are expressed in a spatially and temporally controlled fashion. Expression of individual genes can be shared between biological programs. Expression of individual genes can be shared among different single cell subtypes; however, expression of a biological program may be cell subtype specific or temporally specific (e.g., the biological program is expressed in a cell subtype at a specific time). Multiple biological programs may include the same gene, reflecting the gene's roles in different processes. Expression of a biological program may be regulated by a master switch, such as a nuclear receptor or transcription factor.

As used herein a “signature” or “gene program” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of immune cells that are linked to particular pathological condition (e.g., inflammation), or linked to a particular outcome or progression of the disease (e.g., autoimmunity), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

As used herein, all gene name symbols refer to the gene as commonly known in the art. The examples described herein that refer to the human gene names are to be understood to also encompasses mouse genes, as well as genes in any other organism (e.g., homologous, orthologous genes). Any reference to the gene symbol is a reference made to the entire gene or variants of the gene. Any reference to the gene symbol is also a reference made to the gene product (e.g., protein). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). The signature as described herein may encompass any of the genes described herein.

### Diseases

In certain embodiments, the disease is a viral infection. In certain embodiments, the virus infects a barrier tissue. As used herein a “barrier cell” or “barrier tissues” refers generally to various epithelial tissues of the body such, but not limited to, those that line the respiratory system, digestive system, urinary system, and reproductive system as well as cutaneous systems. The epithelial barrier may vary in composition between tissues but is composed of basal and apical components, or crypt/villus components in the case of intestine.

In certain embodiments, the disease is caused by a differential immune response (e.g., subjects have different immune responses to SARS-CoV-2 which affects severity of COVID-19 disease). In certain embodiments, immune responses are coordinated by immune cells and epithelial cells. The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc. As used throughout this specification, “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”) and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. An immune response can also be an innate immune response (see, e.g., Artis D, Spits H. The biology of innate lymphoid cells. Nature. 2015; 517(7534):293-301).

In certain embodiments, the viral infection is a coronavirus infection. As used herein, “coronavirus” refers to enveloped viruses with a positive-sense single-stranded RNA genome and a nucleocapsid of helical symmetry that constitute the subfamily Orthocoronavirinae, in the family Coronaviridae (see, e.g., Woo P C, Huang Y, Lau S K, Yuen K Y. Coronavirus genomics and bioinformatics analysis. Viruses. 2010; 2(8):1804-1820). The present disclosure relates to and/or involves SARS-CoV-2. Severe acute respiratory syndrome coronavirus 2 (SARS-CoV-2) is the virus causing the ongoing Coronavirus Disease 19 (COVID19) pandemic (see, e.g., Zhou, et al. (2020). A pneumonia outbreak associated with a new coronavirus of probable bat origin. Nature 579, 270-273). In preferred embodiments, the virus is SARS-CoV-2 or variants thereof. In preferred embodiments, the disease treated is COVID-19. SARS-CoV-2 is the third zoonotic betacoronavirus to cause a human outbreak after SARS-CoV in 2002 and Middle East respiratory syndrome coronavirus (MERS-CoV) in 2012 (de Wit et al., 2016, SARS and MERS: recent insights into emerging coronaviruses. Nat Rev Microbiol 14, 523-534). As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain embodiments, a variant has developed a “specific group of mutations” that causes the variant to behave differently than that of the strain it originated from.

While there are many thousands of variants of SARS-CoV-2, (Koyama, Takahiko Koyama; Platt, Daniela; Parida, Laxmi (June 2020). “Variant analysis of SARS-CoV-2 genomes”. Bulletin of the World Health Organization. 98: 495-504) there are also much larger groupings called clades. Several different clade nomenclatures for SARS-CoV-2 have been proposed. As of December 2020, GISAID, referring to SARS-CoV-2 as hCoV-19 identified seven clades (O, S, L, V, G, GH, and GR) (Alm E, Broberg E K, Connor T, et al. Geographical and temporal distribution of SARS-CoV-2 clades in the WHO European Region, January to June 2020 [published correction appears in Euro Surveill. 2020 August; 25(33):]. Euro Surveill. 2020; 25(32):2001410). Also as of December 2020, Nextstrain identified five (19A, 19B, 20A, 20B, and 20C) (Cited in Alm et al. 2020). Guan et al. identified five global clades (G614, S84, V251, 1378 and D392) (Guan Q, Sadykov M, Mfarrej S, et al. A genetic barcode of SARS-CoV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A.; Holmes, E. C.; O'Toole, Á.; et al. “A dynamic nomenclature proposal for SARS-CoV-2 lineages to assist genomic epidemiology”. 5: 1403-1407).

Genetic variants of SARS-CoV-2 have been emerging and circulating around the world throughout the COVID-19 pandemic (see, e.g., The US Centers for Disease Control and Prevention; www.cdc.gov/coronavirus/2019-ncov/variants/variant-info.html). Exemplary, non-limiting variants applicable to the present disclosure include variants of SARS-CoV-2, particularly those having substitutions of therapeutic concern. Table A shows exemplary, non-limiting genetic substitutions in SARS-CoV-2 variants.

TABLE A

| | Common Pango Lineages |
|---|---|
| | with Spike Protein |
| Spike Protein Substitution | Substitutions |
| L452R | A.2.5, B.1, B.1.429, B.1.427, |
| | B.1.617.1, B.1.526.1, |
| | B.1.617.2, C.36.3 |
| E484K | B.1.1.318, B.1.1.7, B.1.351, |
| | B.1.525, B.1.526, B.1.621, |
| | B.1.623, P.1, P.1.1, P.1.2, |
| | R.1 |
| K417N, E484K, N501Y | B.1.351, B.1.351.3 |
| K417T, E484K, N501Y | P.1, P.1.1, P.1.2 |
| A67V, del69-70, T95I, del142-144, | B.1.1.529 and BA lineages |
| Y145D, del211, L212I, ins214EPE, | |
| G339D, S371L, S373P, S375F, K417N, | |
| N440K, G446S, S477N, T478K, E484A, | |
| Q493R, G496S, Q498R, N501Y, Y505H, | |
| T547K, D614G, H655Y, N679K, P681H, | |
| N764K, D796Y, N856K, Q954H, N969K, | |
| L981F | |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-CoV-2 lineages, known as the PANGO nomenclature. It is available at cov-lineages.org.

In some embodiments, the SARS-CoV-2 variant is and/or includes: B.1.1.7, also known as Alpha (WHO) or UK variant, having the following spike protein substitutions: 69del, 70del, 144del, (E484K*), (S494P*), N501Y, A570D, D614G, P681H, T7161, S982A, and D1118H (K1191N*); B.1.351, also known as Beta (WHO) or South Africa variant, having the following spike protein substitutions: D80A, D215G, 241del, 242del, 243del, K417N, E484K, N501Y, D614G, and A701V; B.1.427, also known as Epsilon (WHO) or US California variant, having the following spike protein substitutions: L452R, and D614G; B.1.429, also known as Epsilon (WHO) or US California variant, having the following spike protein substitutions: S13I, W152C, L452R, and D614G; B.1.617.2, also known as Delta (WHO) or India variant, having the following spike protein substitutions: T19R, (G142D), 156del, 157del, R158G, L452R, T478K, D614G, P681R, and D950N; P.1, also known as Gamma (WHO) or Japan/Brazil variant, having the following spike protein substitutions: L18F, T20N, P26S, D138Y, R190S, K417T, E484K, N501Y, D614G, H655Y, and T1027I; and B.1.1.529 also known as Omicron (WHO), having the following spike protein substitutions: A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, or any combination thereof.

In some embodiments, the SARS-CoV-2 variant is classified and/or otherwise identified as a Variant of Concern (VOC) by the World Health Organization and/or the U.S. Centers for Disease Control. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures.

In some embodiments, the SARS-Cov-2 variant is classified and/or otherwise identified as a Variant of High Consequence (VHC) by the World Health Organization and/or the U.S. Centers for Disease Control. A variant of high consequence has clear evidence that prevention measures or medical countermeasures (MCMs) have significantly reduced effectiveness relative to previously circulating variants.

In some embodiments, the SARS-Cov-2 variant is classified and/or otherwise identified as a Variant of Interest (VOI) by the World Health Organization and/or the U.S. Centers for Disease Control. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

In some embodiments, the SARS-Cov-2 variant is classified and/or is otherwise identified as a Variant of Note (VON). As used herein, VON refers to both “variants of concern” and “variants of note” as the two phrases are used and defined by Pangolin (cov-lineages.org) and provided in their available “VOC reports” available at cov-lineages.org.

In some embodiments the SARS-Cov-2 variant is a VOC. In some embodiments, the SARS-CoV-2 variant is or includes an Alpha variant (e.g., Pango lineage B.1.1.7), a Beta variant (e.g., Pango lineage B.1.351, B.1.351.1, B.1.351.2, and/or B.1.351.3), a Delta variant (e.g., Pango lineage B.1.617.2, AY.1, AY.2, AY.3 and/or AY.3.1); a Gamma variant (e.g., Pango lineage P.1, P.1.1, P.1.2, P.1.4, P.1.6, and/or P.1.7), an Omicron variant (B.1.1.529) or any combination thereof.

In some embodiments the SARS-Cov-2 variant is a VOL. In some embodiments, the SARS-CoV-2 variant is or includes an Eta variant (e.g., Pango lineage B.1.525 (Spike protein substitutions A67V, 69del, 70del, 144del, E484K, D614G, Q677H, F888L)); an Iota variant (e.g., Pango lineage B.1.526 (Spike protein substitutions L5F, (D80G*), T95I, (Y144-*), (F157S*), D253G, (L452R*), (S477N*), E484K, D614G, A701V, (T859N*), (D950H*), (Q957R*))); a Kappa variant (e.g., Pango lineage B.1.617.1 (Spike protein substitutions (T951), G142D, E154K, L452R, E484Q, D614G, P681R, Q1071H)); Pango lineage variant B.1.617.2 (Spike protein substitutions T19R, G142D, L452R, E484Q, D614G, P681R, D950N)), Lambda (e.g., Pango lineage C.37); or any combination thereof.

In some embodiments SARS-Cov-2 variant is a VON. In some embodiments, the SARS-Cov-2 variant is or includes Pango lineage variant P.1 (alias, B.1.1.28.1.) as described in Rambaut et al. 2020. Nat. Microbiol. 5:1403-1407) (spike protein substitutions: T20N, P26S, D138Y, R190S, K417T, E484K, N501Y, H655Y, TI027I)); an Alpha variant (e.g., Pango lineage B.1.1.7); a Beta variant (e.g., Pango lineage B.1.351, B.1.351.1, B.1.351.2, and/or B.1.351.3); Pango lineage variant B.1.617.2 (Spike protein substitutions T19R, G142D, L452R, E484Q, D614G, P681R, D950N)); an Eta variant (e.g., Pango lineage B.1.525); Pango lineage variant A.23.1 (as described in Bugembe et al. medRxiv. 2021. doi: https://doi.org/10.1101/2021.02.08.21251393) (spike protein substitutions: F157L, V367F, Q613H, P681R); or any combination thereof.

### Diagnostic Methods

In certain embodiments, detecting cell subset markers or differentially expressed genes can be used to determine a treatment for a subject suffering from a disease or stratify a subject based on risk of developing severe disease (e.g., COVID-19). The invention provides biomarkers (e.g., phenotype specific or cell subtype) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations who would benefit from treatment based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the cells in the subject compared to a control subject having normal status or not having a disease indicates response to treatment. Hence, the methods may rely on comparing the quantity of cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or 1×SE or ±2×SE or ±3×SE). Deviation may also refer to a value falling outside of a reference range defined by values in a given population (for example, outside of a range which comprises >40%, >50%, >60%, >70%, >75% or >80% or >85% or >90% or >95% or even >100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

### Stratification of Subjects

In certain embodiments, the subject is determined to belong to or at risk to progress to the severe risk group if one or more (e.g., 1, 2, 3, 4, 5, 6, 7, 8, 9, 10, etc. or more) of proinflammatory cytokines comprising at least one or more of: IL1B, TNF, CXCL8, CCL2, CCL3, CXCL9, CXCL10, and CXCL11; upregulation of alarmins comprising one or both of: S100A8 and S100A9; 14%-26% of all epithelial cells are secretory cells; elevated BPIFA1 high Secretory cells; elevated KRT13 KRT24 high secretory cells; macrophage population increase as compared to other immune cells; upregulated genes in ciliated cells comprising one or both of: IL5RA and NLRP1; no increase of at least one or more of: type I, type II, and type III interferon abundance; elevated stress response factors comprising at least one or more of: HSPA8, HSPA1A, and DUSP1; and reduced or absent antiviral/interferon response, and reduced or absent mature ciliated cells is detected. In certain embodiments, the subject is determined to belong to the mild/moderate risk group if one or more (e.g., 1, 2, 3, 4, 5, 6, 7, 8, 9, 10, etc. or more) of 4%-12% of all epithelial cells are Secretory Cells; 10%-20% of all epithelial cells comprise Interferon Responsive Ciliated Cells; upregulated ciliated cell genes comprising at least one or more of: IFI44L, STAT1, IFITM1, MX1, IFITM3, OAS1, OAS2, OAS3, STAT2, TAP1, HLA-C, ADAR, XAF1, IRF1, CTSS, and CTSB; increase in type I interferon abundance; high expression of interferon-responsive genes; induction of type I interferon responses; and high abundance of IFI6 and IFI27 is detected.

In certain embodiments, one or more (e.g., 1, 2, 3, 4, 5, 6, 7, 8, 9, 10, etc. or more) cell subset markers or differentially expressed genes found in Table 2 are detected in a sample from a subject stratify the subject into the mild/moderate risk group. In certain embodiments, one or more (e.g., 1, 2, 3, 4, 5, 6, 7, 8, 9, 10, etc. or more) cell subset markers or differentially expressed genes found in Table 3 are detected in a sample from a subject stratify the subject into the severe risk group. In certain embodiments, one or more (e.g., 1, 2, 3, 4, 5, 6, 7, 8, 9, 10, etc. or more) cell subset markers or differentially expressed genes found in Table 3 are detected in a sample from a subject stratify the subject into the mild/moderate risk group or severe risk group. In certain embodiments, one or more (e.g., 1, 2, 3, 4, 5, 6, 7, 8, 9, 10, etc. or more) cell subset markers or differentially expressed genes found in Table 5 are detected in a sample from a subject stratify the subject into the risk of developing the disease or having the disease.

### Sample Collection

In some embodiments, a sample can be collected with a nasal swab, endoscopy, polyester tipped swabs, plastic curettes, cytology brushes (Lai P S, et al. J Allergy Clin Immunol. 2015; 136(4)). Tissue samples for diagnosis, prognosis or detecting may be obtained by endoscopy. In one embodiment, a sample may be obtained by endoscopy and analyzed b FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample. A cutting tool can be attached to the end of the endoscope, and the apparatus can then be used to perform surgery. Applications of endoscopy that can be used with the present invention include, but are not limited to examination of the oesophagus, stomach and duodenum (esophagogastroduodenoscopy); small intestine (enteroscopy); large intestine/colon (colonoscopy, sigmoidoscopy); bile duct; rectum (rectoscopy) and anus (anoscopy), both also referred to as (proctoscopy); respiratory tract; nose (rhinoscopy); lower respiratory tract (bronchoscopy); ear (otoscope); urinary tract (cystoscopy); female reproductive system (gynoscopy); cervix (colposcopy); uterus (hysteroscopy); fallopian tubes (falloposcopy); normally closed body cavities (through a small incision); abdominal or pelvic cavity (laparoscopy); interior of a joint (arthroscopy); or organs of the chest (thoracoscopy and mediastinoscopy).

In one non-limiting example, nasopharyngeal samples are collected by a trained healthcare provider using FLOQSwabs (Copan 1109 flocked swabs) following the manufacturer's instructions. Collectors don personal protective equipment (PPE), including a gown, non-sterile gloves, a protective N95 mask, a bouffant, and a face shield. The patient's head is tilted back slightly, and the swab is inserted along the nasal septum, above the floor of the nasal passage to the nasopharynx until slight resistance was felt. The swab is then left in place for several seconds to absorb secretions and is slowly removed while rotating swab. The swab is then placed into a cryogenic vial with 900 μL of heat inactivated fetal bovine serum (FBS) and 100 μL of dimethyl sulfoxide (DMSO). Vials are placed into a Mr. Frosty Freezing Container (Thermo Fisher Scientific) for optimal cell preservation. A Mr. Frosty containing the vials is placed in a cooler with dry ice for transportation from patient areas to the laboratory for processing. Once in the laboratory, the Mr. Frosty is placed into a −80° C. freezer overnight, and on the next day, the vials are moved to liquid nitrogen storage containers.

In one non-limiting example, swabs in freezing media (90% FBS/10% DMSO) were stored in liquid nitrogen until immediately prior to dissociation. This approach ensures that all cells and cellular material from the nasal swab (whether directly attached to the nasal swab, or released during the washing and digestion process), are exposed first to DTT for 15 minutes, followed by an Accutase digestion for 30 minutes. Briefly, nasal swabs in freezing media were thawed, and each swab was rinsed in RPMI before incubation in 1 mL RPMI/10 mM DTT (Sigma) for 15 minutes at 37° C. with agitation. Next, the nasal swab was incubated in 1 mL Accutase (Sigma) for 30 minutes at 37° C. with agitation. The 1 mL RPMI/10 mM DTT from the nasal swab incubation was centrifuged at 400 g for 5 minutes at 4° C. to pellet cells, the supernatant was discarded, and the cell pellet was resuspended in 1 mL Accutase and incubated for 30 minutes at 37° C. with agitation. The original cryovial containing the freezing media and the original swab washings were combined and centrifuged at 400 g for 5 minutes at 4° C. The cell pellet was then resuspended in RPMI/10 mM DTT, and incubated for 15 minutes at 37° C. with agitation, centrifuged as above, the supernatant was aspirated, and the cell pellet was resuspended in 1 mL Accutase, and incubated for 30 minutes at 37° C. with agitation. All cells were combined following Accutase digestion and filtered using a 70 m nylon strainer. The filter and swab were washed with RPMI/10% FBS/4 mM EDTA, and all washings combined. Dissociated, filtered cells were centrifuged at 400 g for 10 minutes at 4° C., and resuspended in 200 μL RPMI/10% FBS for counting. Cells were diluted to 20,000 cells in 200 μL for scRNA-seq. For the majority 1140 of swabs, fewer than 20,000 cells total were recovered. In these instances, all cells were input into scRNA-seq.

### Detection of Biomarkers

In one embodiment, the signature genes, biomarkers, and/or cells may be detected by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) (Chen et al., Spatially resolved, highly multiplexed RNA profiling in single cells. Science, 2015, 348:aaa6090; and Xia et al., Multiplexed detection of RNA using MERFISH and branched DNA amplification. Sci Rep. 2019 May 22; 9(1):7721. doi: 10.1038/s41598-019-43943-8), ExSeq (Alon, S. et al. Expansion Sequencing: Spatially Precise In Situ Transcriptomics in Intact Biological Systems. biorxiv.org/lookup/doi/10.1101/2020.05.13.094268 (2020) doi:10.1101/2020.05.13.094268), and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed by FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample (e.g., a biopsy).

The present invention also may comprise a kit with a detection reagent that binds to one or more biomarkers or can be used to detect one or more biomarkers.

Immunoassays Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B. V. (1993) and Kricka, “Nonisotopic DNA Probe Techniques”, Academic Press, San Diego, Calif. (1992).

### Single Cell Sequencing

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Therapeutic Methods

### Treatment Selection

In certain embodiments, the methods of the present invention are used to select a treatment within the current standard of care and provide for less toxicity and improved treatment. The term “standard of care” as used herein refers to the current treatment that is accepted by medical experts as a proper treatment for a certain type of disease and that is widely used by healthcare professionals. Standard of care is also called best practice, standard medical care, and standard therapy. In certain embodiments, a subject having a mild or moderate phenotype will recover without any treatment. In certain embodiments, a subject having a severe phenotype requires treatment in order to recover. In certain embodiments, severe subjects or subjects at risk for severe disease as determined by detecting cell subsets and/or differentially expressed genes are treated with one or more agents as described further herein. In certain embodiments, subjects already suffering from severe disease are treated. In certain embodiments, subjects at risk for severe disease are treated. In certain embodiments, the treatment results in induction of a phenotype identified in mild/moderate subjects (e.g., antiviral response).

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

In certain embodiments, the therapeutic agents are administered in an effective amount or therapeutically effective amount. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

### Therapeutic Agents

In certain embodiments, the present invention provides for one or more therapeutic agents capable of shifting a phenotype as described herein. In certain embodiments, the present invention provides for one or more therapeutic agents against one or more of the targets identified. In certain embodiments, the one or more agents comprises a small molecule inhibitor, small molecule degrader (e.g., ATTEC, AUTAC, LYTAC, or PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, the therapeutic agents are administered in an effective amount or therapeutically effective amount. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

In certain embodiments, an agent against one of the targets is used in combination with a treatment already be known or used clinically. In certain embodiments, targeting the combination may require less of the agent as compared to the current standard of care and provide for less toxicity and improved treatment.

### Antiviral

In certain embodiments, the one or more agent is an antiviral. In certain embodiments, an antiviral inhibits viral replication. In certain embodiments, the antiviral is paxlovid. The U.S. Food and Drug Administration issued an emergency use authorization (EUA) for Pfizer's Paxlovid (nirmatrelvir tablets and ritonavir tablets, co-packaged for oral use) for the treatment of mild-to-moderate coronavirus disease (COVID-19) in adults and pediatric patients (12 years of age and older weighing at least 40 kilograms or about 88 pounds) with positive results of direct SARS-CoV-2 testing, and who are at high risk for progression to severe COVID-19, including hospitalization or death (Paxlovid EUA Letter of Authorization issued Dec. 22, 2021). In certain embodiments, the antiviral is molnupiravir. The U.S. Food and Drug Administration issued an emergency use authorization (EUA) for Merck's molnupiravir for the treatment of mild-to-moderate coronavirus disease (COVID-19) in adults with positive results of direct SARS-CoV-2 viral testing, and who are at high risk for progression to severe COVID-19, including hospitalization or death, and for whom alternative COVID-19 treatment options authorized by the FDA are not accessible or clinically appropriate (Molnupiravir EUA Letter of Authorization issued Feb. 11, 2022). In certain embodiments, the antiviral is Remdesivir.

### Immune-Based Therapy

In certain embodiments, the one or more agent is immune-based therapy. In certain embodiments, the immune-based therapy is a blood-derived product. In certain embodiments, the blood-derived product is convalescent plasma. In certain embodiments, the blood-derived product is immunoglobulin. In certain embodiments, the immune-based therapy is immunoglobin. In certain embodiments, the immune-based therapy is one or more of: a corticosteroid, a glucocorticoid, an interferon, an interferon Type I agonist, an interleukin-1 inhibitor, an interleukin-6 inhibitor, a kinase inhibitor, and a TLR agonist. In certain embodiments, the corticosteroid comprises at least one of: methylprednisolone, hydrocortisone, and dexamethasone. In certain embodiments, the glucocorticoid comprises at least one of: cortisone, prednisone, prednisolone, methylprednisolone, dexamethasone, betamethasone, triamcinolone, Fludrocortisone acetate, deoxycorticosterone acetate, and hydrocortisone. In certain embodiments, the interferon comprises at least one or more of: interferon beta-1b and interferon alpha-2b. In certain embodiments, the interleukin-1 inhibitor comprises anakinra. In certain embodiments, the interleukin-6 inhibitor comprises at least one or more of: anti-interleukin-6 receptor monoclonal antibodies and anti-interleukin-6 monoclonal antibody. In certain embodiments, the anti-interleukin-6 receptor monoclonal antibody is tocilizumab. In certain embodiments, the anti-interleukin-6 monoclonal antibody is siltuximab. In certain embodiments, the kinase inhibitor comprises of at least one or more of Bruton's tyrosine kinase inhibitor and Janus kinase inhibitor. In certain embodiments, the Bruton's tyrosine kinase inhibitor comprises at least one or more of: acalabrutinib, ibrutinib, and zanubrutinib. In certain embodiments, the Janus kinase inhibitor comprises at least one or more of: baracitinib, ruxolitinib and tofacitinib. In certain embodiments, the TLR agonist comprises at least one or more of: imiquimod, BCG, and MPL.

### Other Treatment Options

In certain embodiments, the treatment comprises inhibiting cholesterol biosynthesis. In certain embodiments, inhibiting cholesterol biosynthesis comprises administering HMG-CoA reductase inhibitors. in certain embodiments, the HMG-CoA reductase inhibitor comprises at least one or more of: simvastatin atorvastatin, lovastatin, pravastatin, fluvastatin, rosuvastatin, pitavastatin. In certain embodiments, wherein the treatment comprises one or more agents capable of shifting epithelial cells to express an antiviral signature. In certain embodiments, the treatment comprises one or more agents capable of suppressing a myeloid inflammatory response.

### Antibodies

In certain embodiments, the one or more agent is an antibody. In certain embodiments, an antibody targets one or more surface genes or polypeptides. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, 1gM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by p pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (1° F.n3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×107 M⁻¹ (or a dissociation coefficient of 1 M or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}—Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds (e.g., CD160). In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Bispecific Antibodies

In certain embodiments, bispecific antibodies are used to target specific cell types (e.g., viral infected cells). Bi-specific antigen-binding constructs, e.g., bi-specific antibodies (bsAb) or BiTEs, bind two antigens (see, e.g., Suurs et al., A review of bispecific antibodies and antibody constructs in oncology and clinical challenges. Pharmacol Ther. 2019 September; 201:103-119; and Huehls, et al., Bispecific T cell engagers for cancer immunotherapy. Immunol Cell Biol. 2015 March; 93(3): 290-296). The bi-specific antigen-binding construct includes two antigen-binding polypeptide constructs, e.g., antigen binding domains. In some embodiments, the antigen-binding construct is derived from known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

### Aptamers

In certain embodiments, the one or more agent is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2-NH₂), 2′-fluoro (2-F), and/or 2-O-methyl (2-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to, those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins as described herein (e.g., CD160).

### Genetic Modifying Agents

In certain embodiments, the one or more modulating agents may be a genetic modifying agent. The genetic modifying agents may manipulate nucleic acids (e.g., genomic DNA or mRNA). The genetic modulating agent can be used to up- or downregulate expression of a gene either by targeting a nuclease or functional domain to a DNA or RNA sequence. The genetic modifying agent may comprise an RNA-guided nuclease system (e.g., CRISPR system), RNAi system, a zinc finger nuclease, a TALE, or a meganuclease. In certain embodiments, one or more genes capable of shifting cell composition or cell states is modified by a genetic modifying agent (e.g., one or more genes in Tables 1-5). In certain embodiments, a genetic modifying agent is used in subjects already having severe disease.

### CRISPR-Cas Modification

In some embodiments, a polynucleotide of the present invention described elsewhere herein can be modified using a CRISPR-Cas and/or Cas-based system (e.g., genomic DNA or mRNA, preferably, for a disease gene). The nucleotide sequence may be or encode one or more components of a CRISPR-Cas system. For example, the nucleotide sequences may be or encode guide RNAs. The nucleotide sequences may also encode CRISPR proteins, variants thereof, or fragments thereof.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

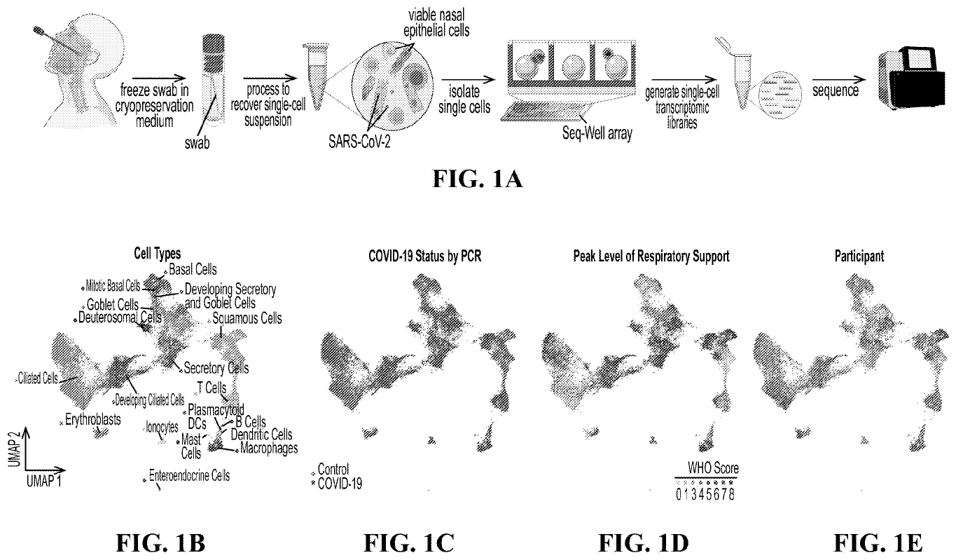

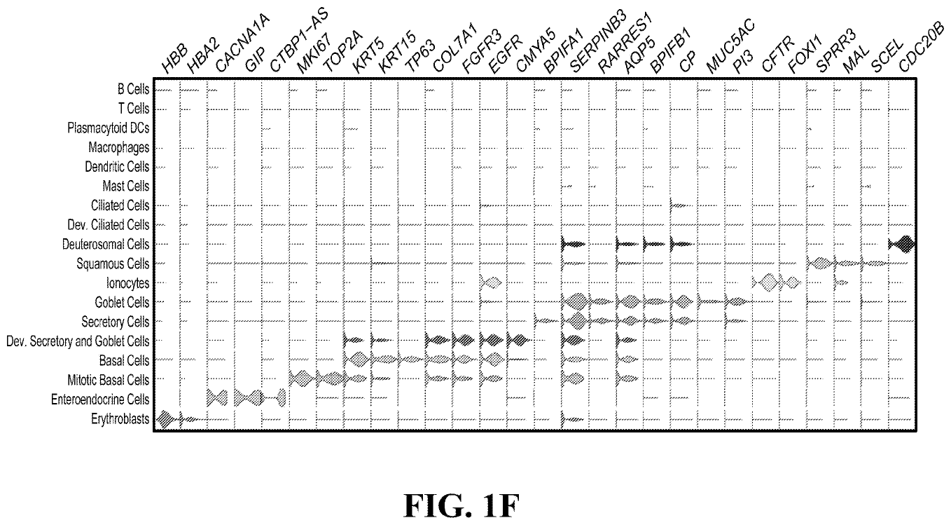

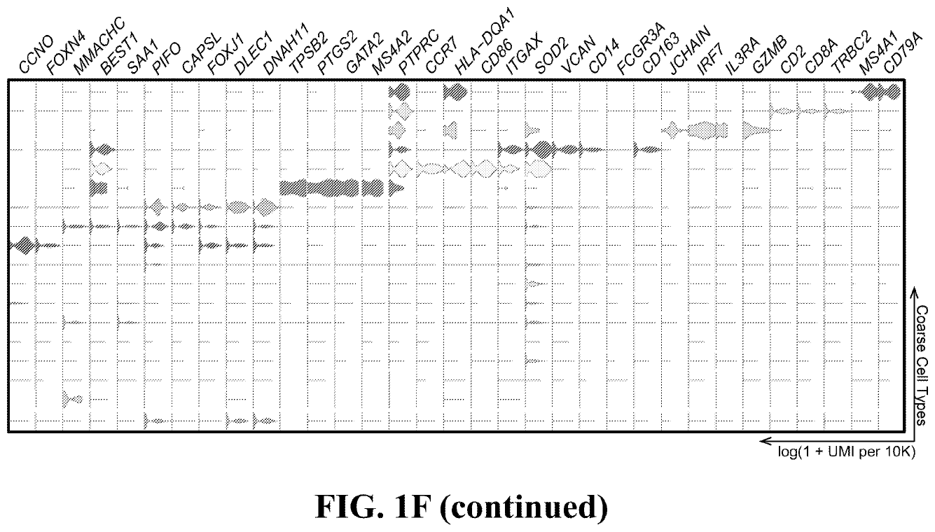

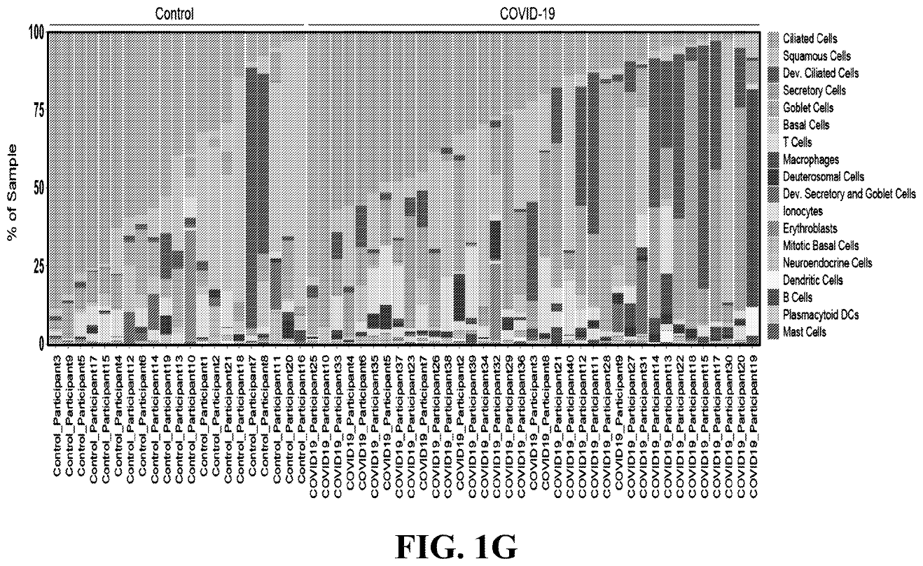

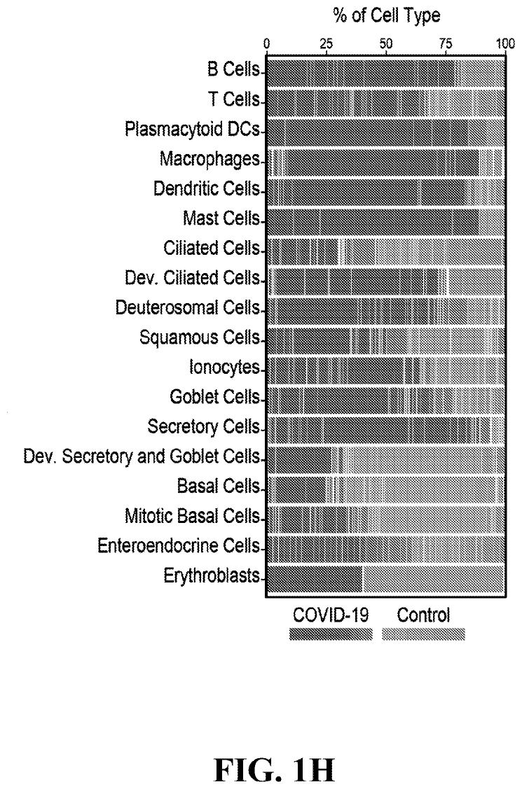

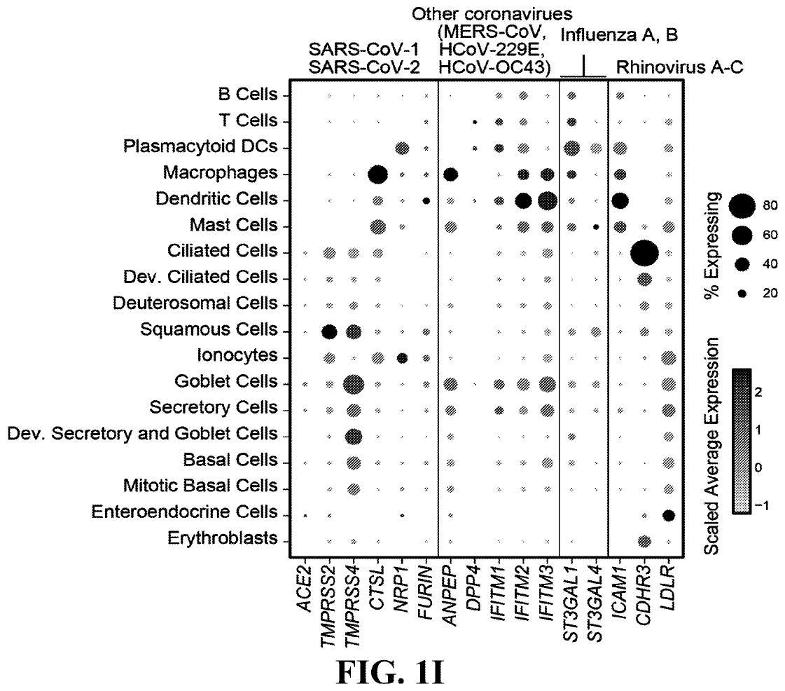

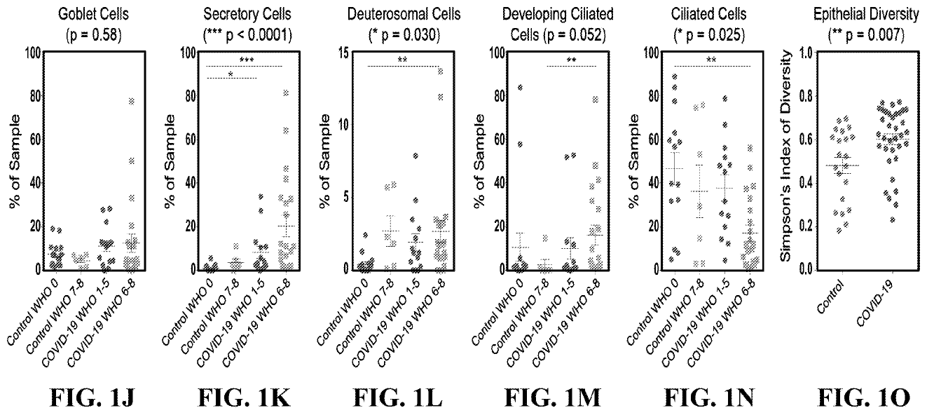

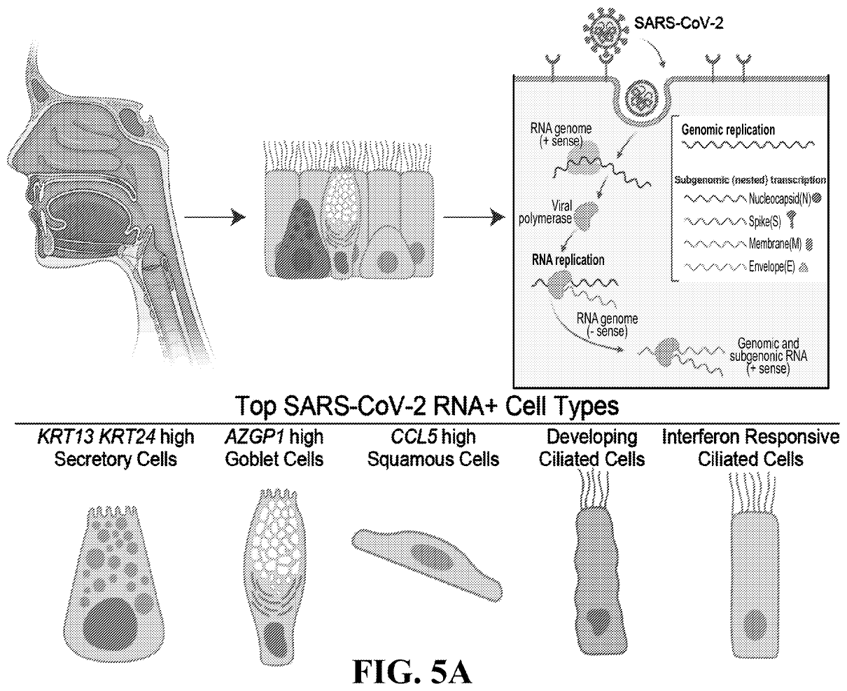

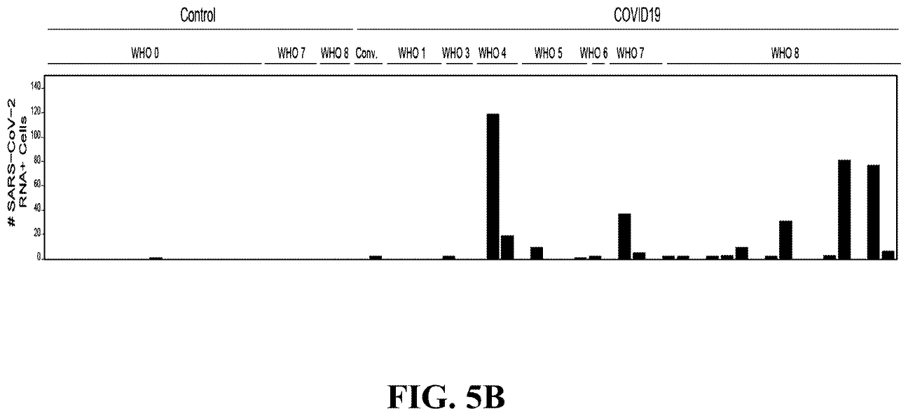

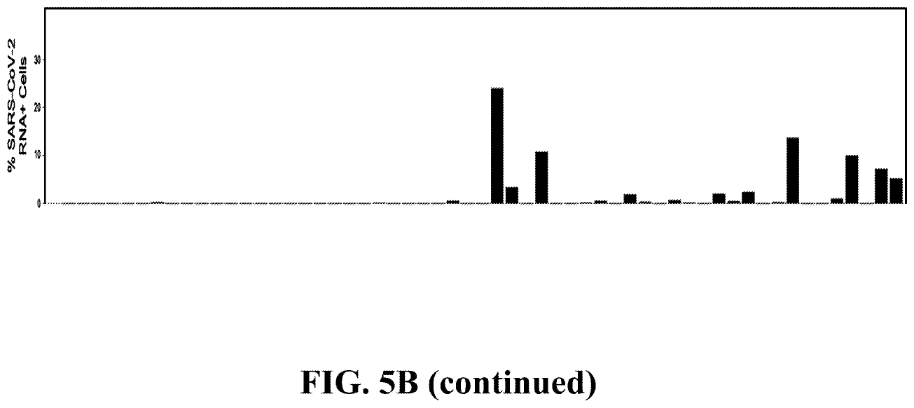

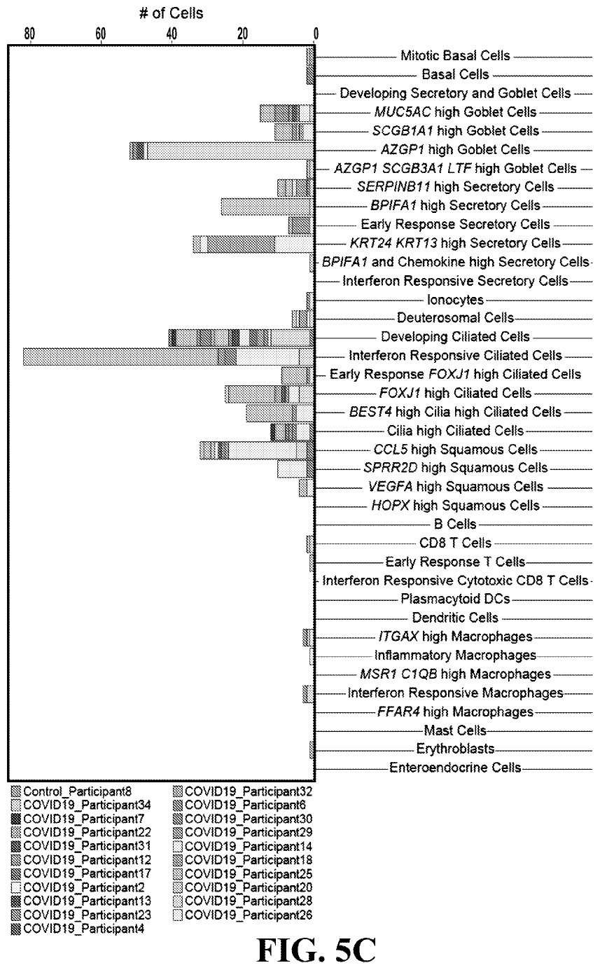

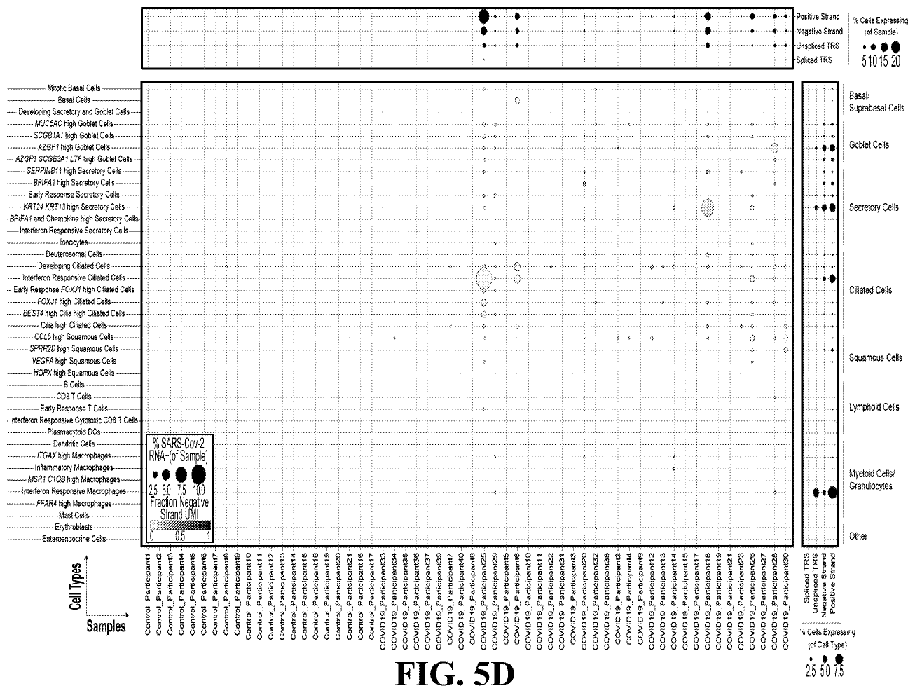

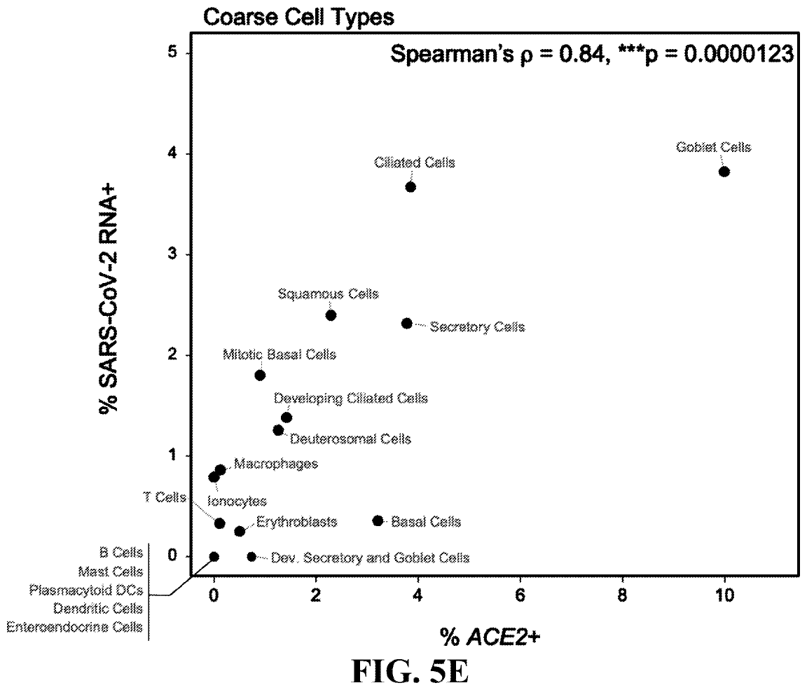

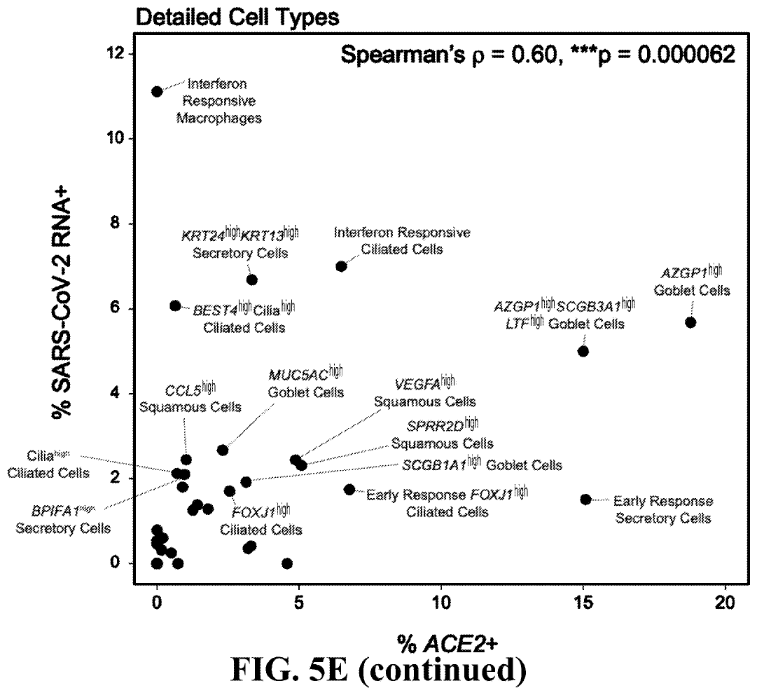

In some embodiments, the CRISPR-Cas system that can be used to modify a polynucleotide of the present invention described herein can be a Class 1 CRISPR-Cas system. Class 1 CRISPR-Cas systems are divided into Types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

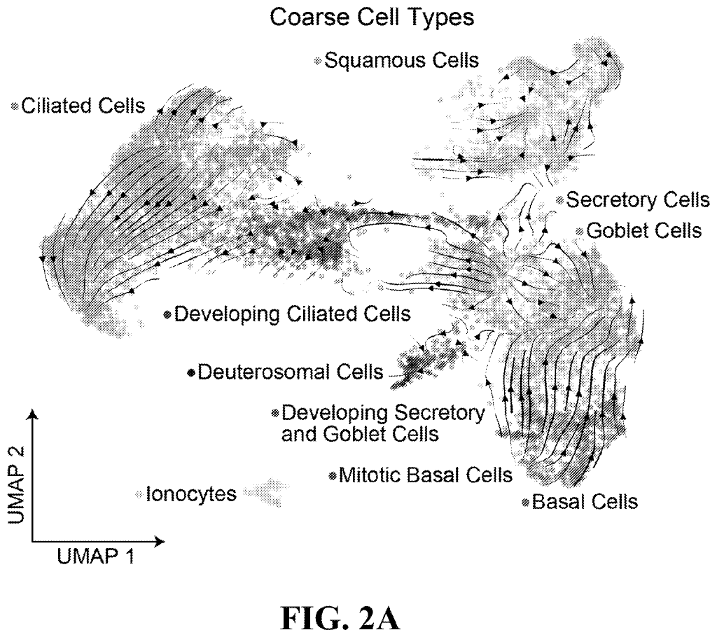

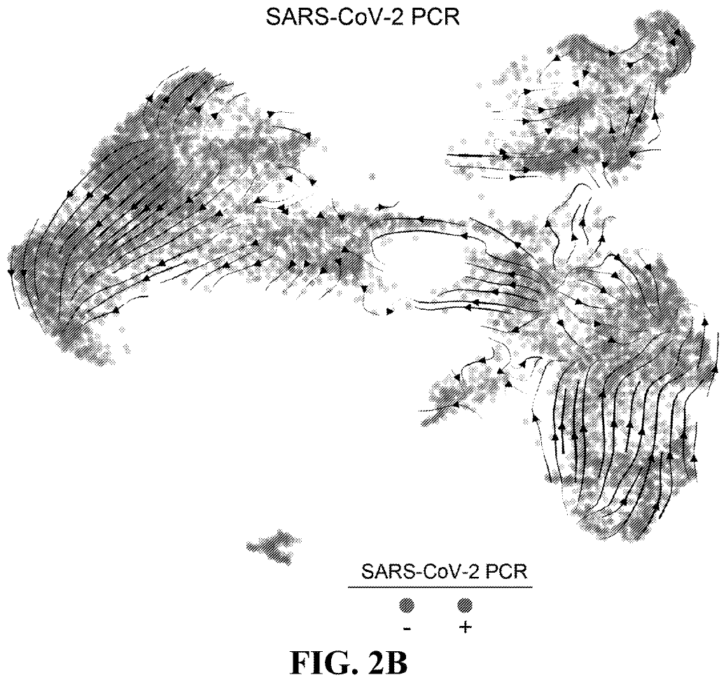

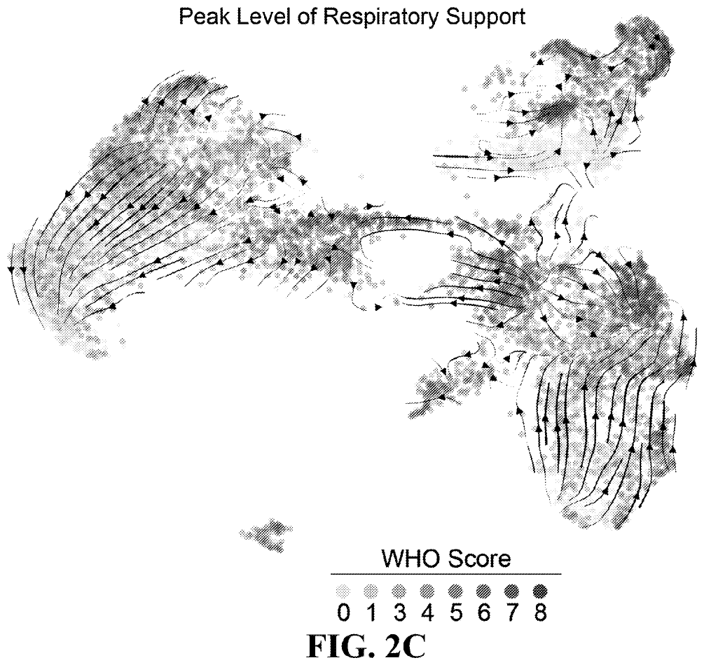

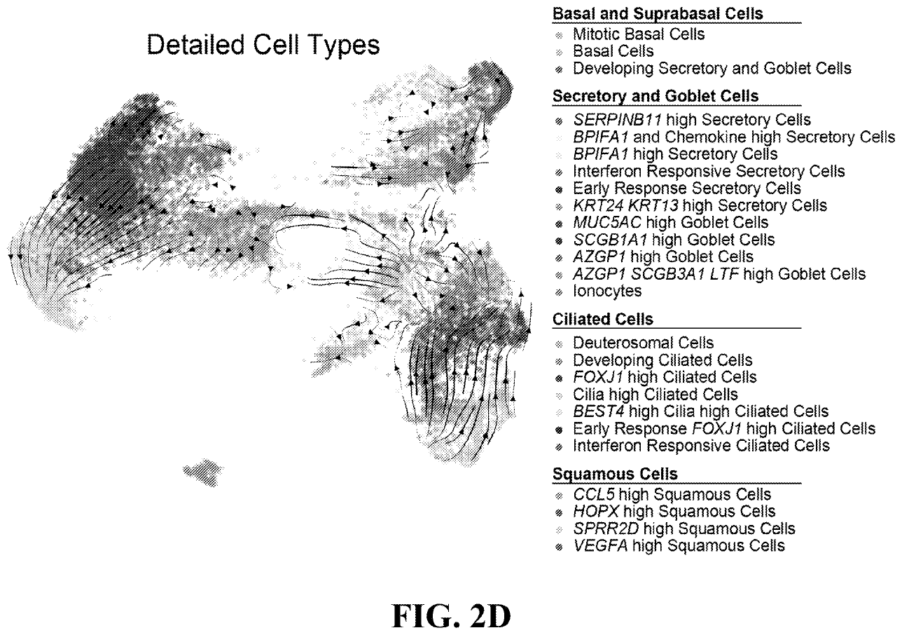

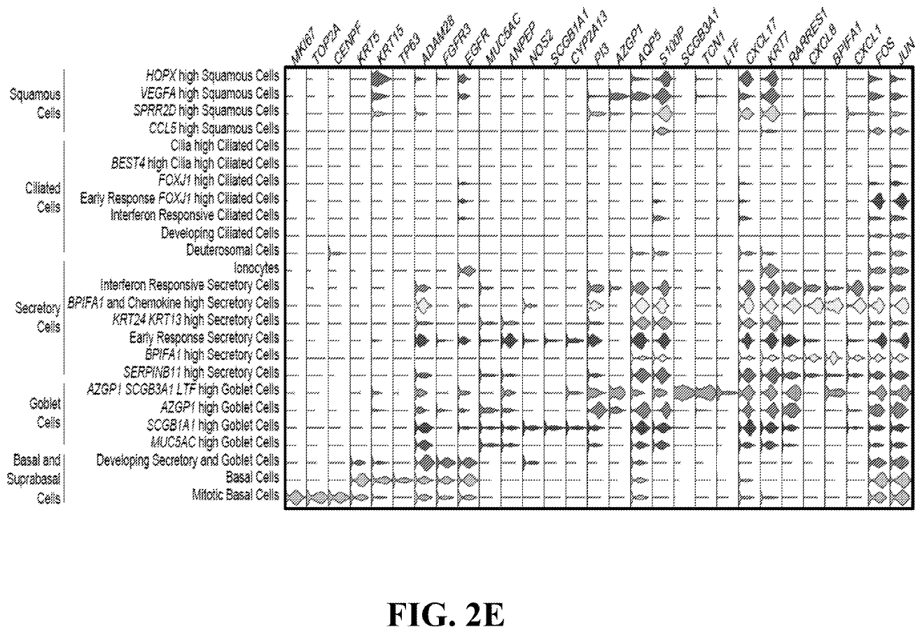

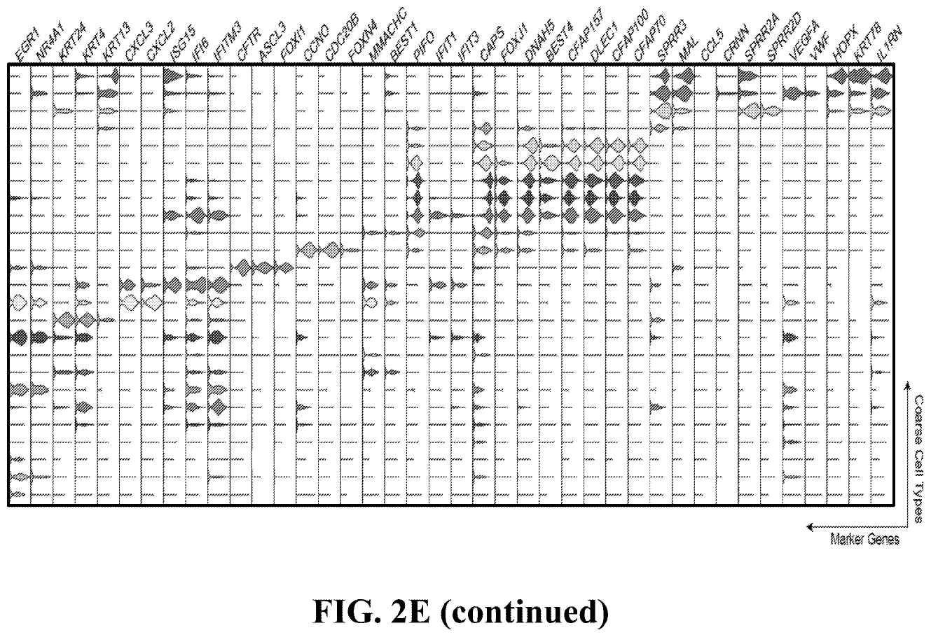

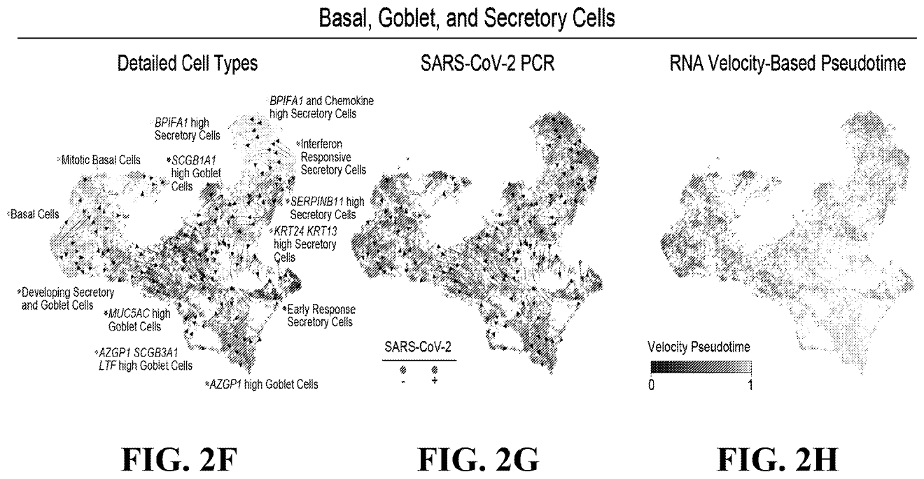

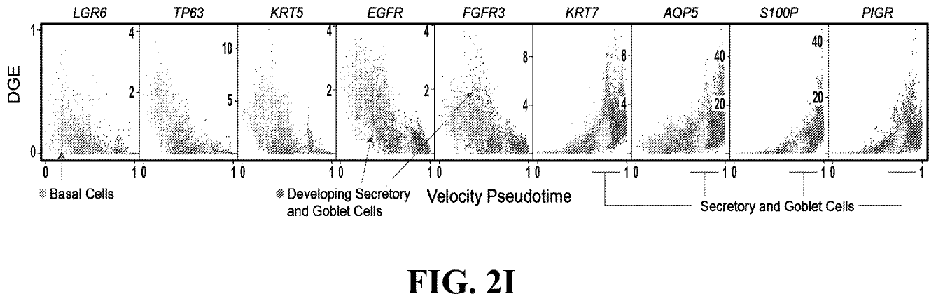

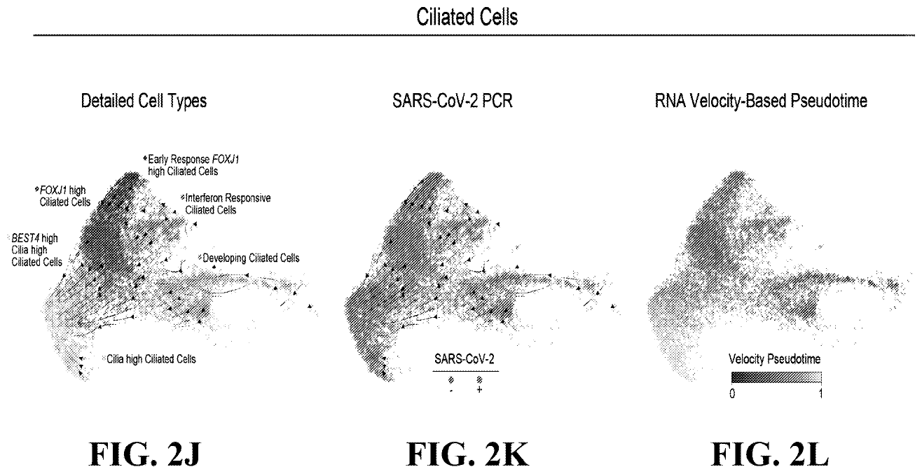

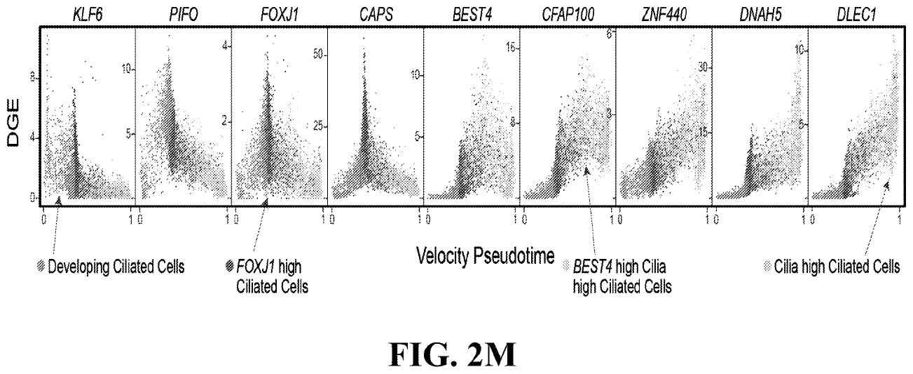

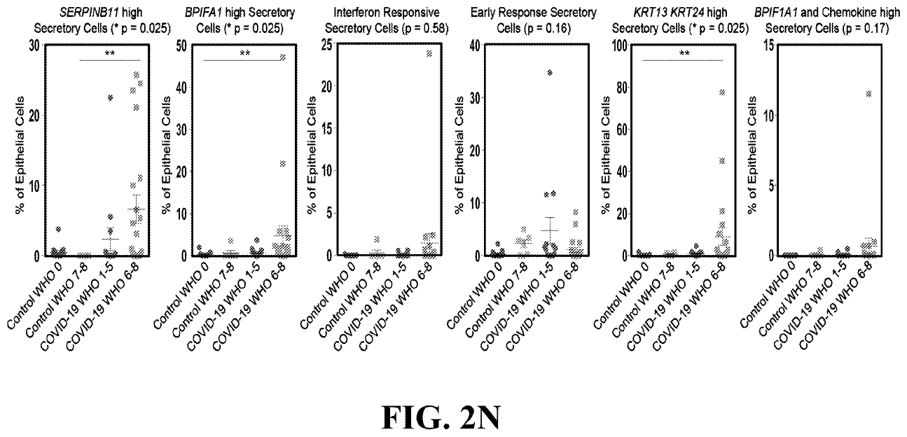

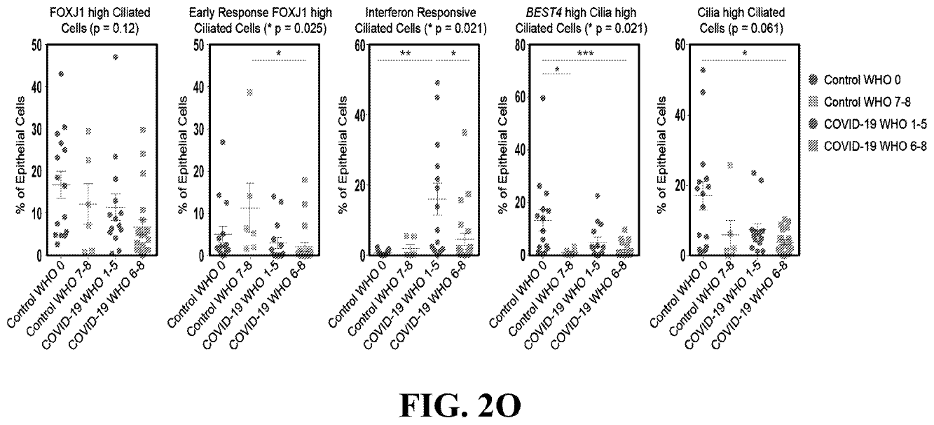

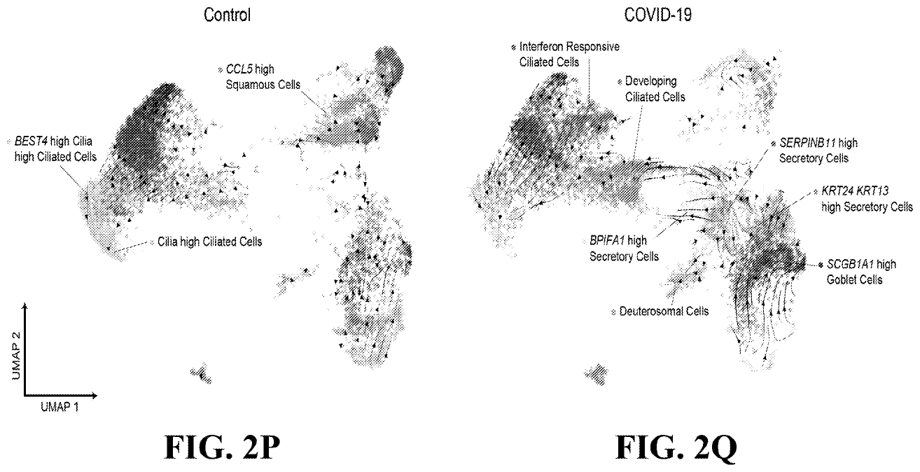

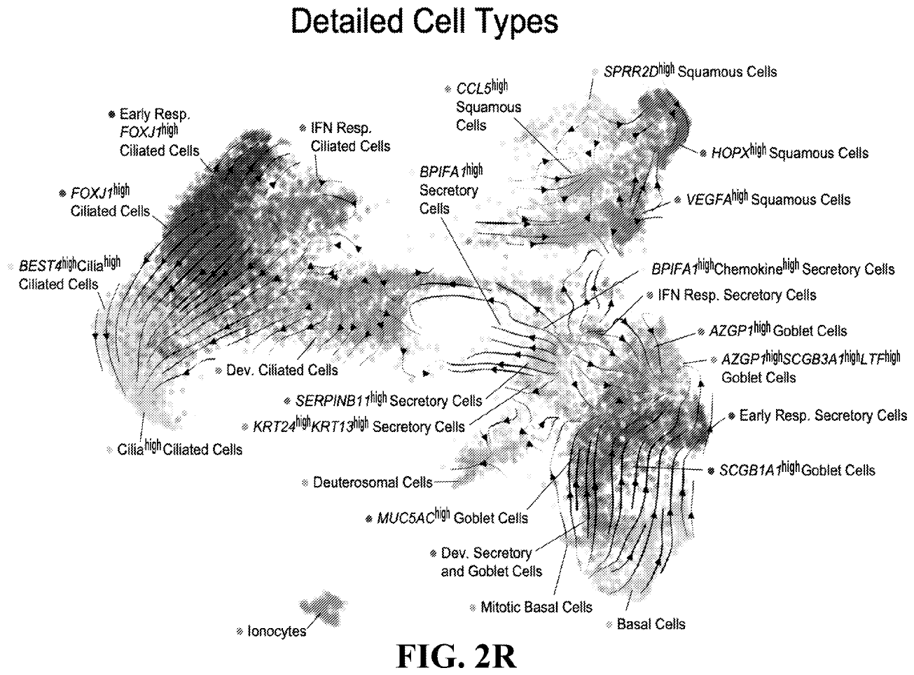

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Etched et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C●G base pair into a T●A base pair (Komor et al. 2016. Nature. 533:420-424; Nishida et al. 2016. Science. 353; and Li et al. Nat. Biotech. 36:324-327) and ABEs convert an A●T base pair to a G●C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g., A:T to G:C). Richter et al. 2020. Nature Biotechnology. doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system (See e.g., Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

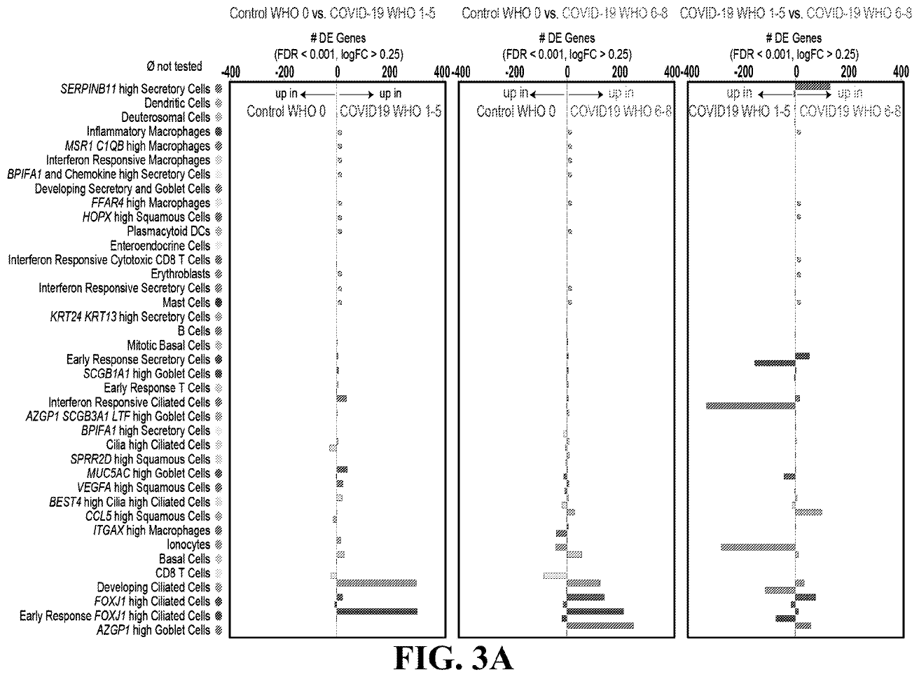

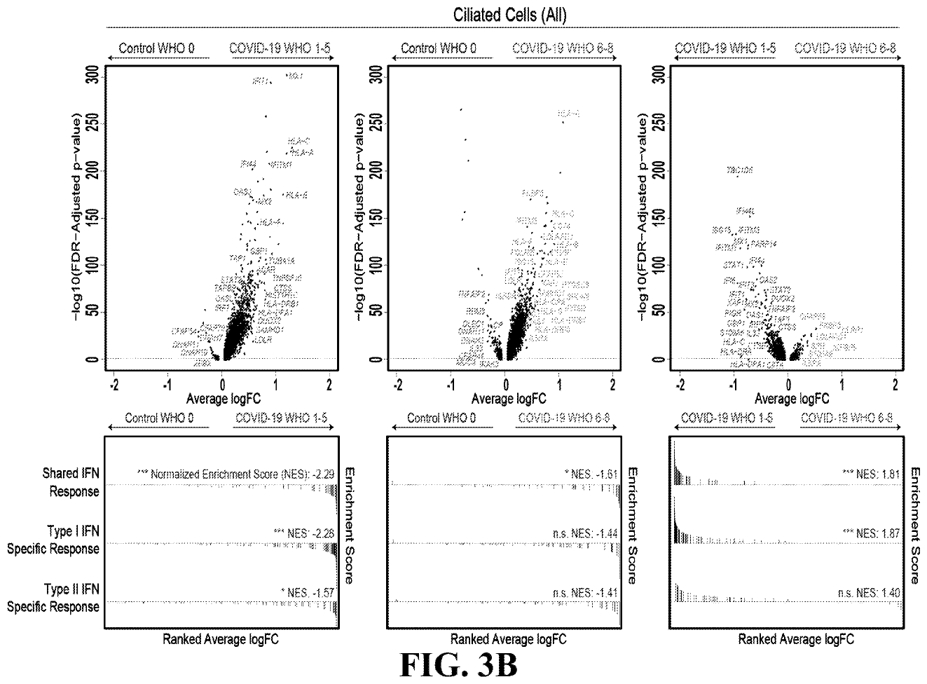

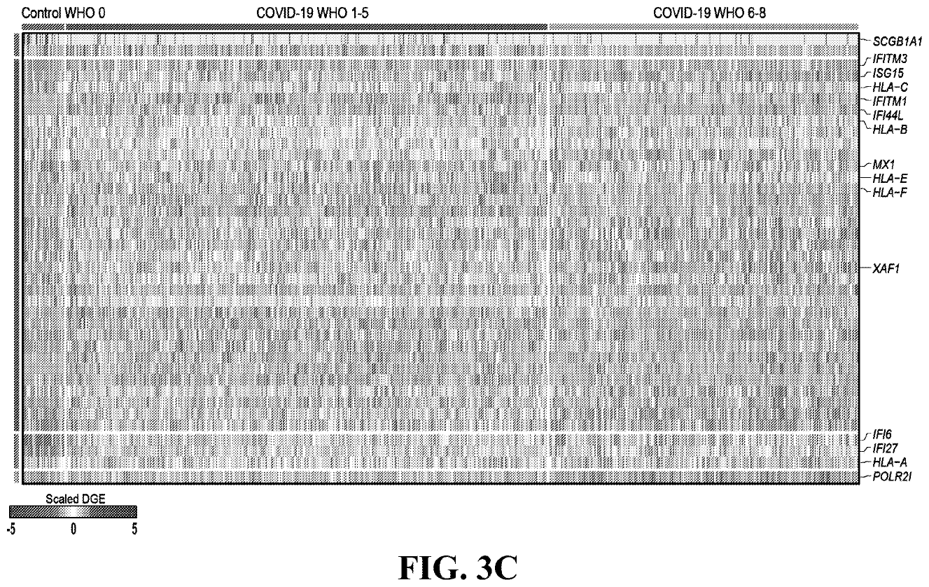

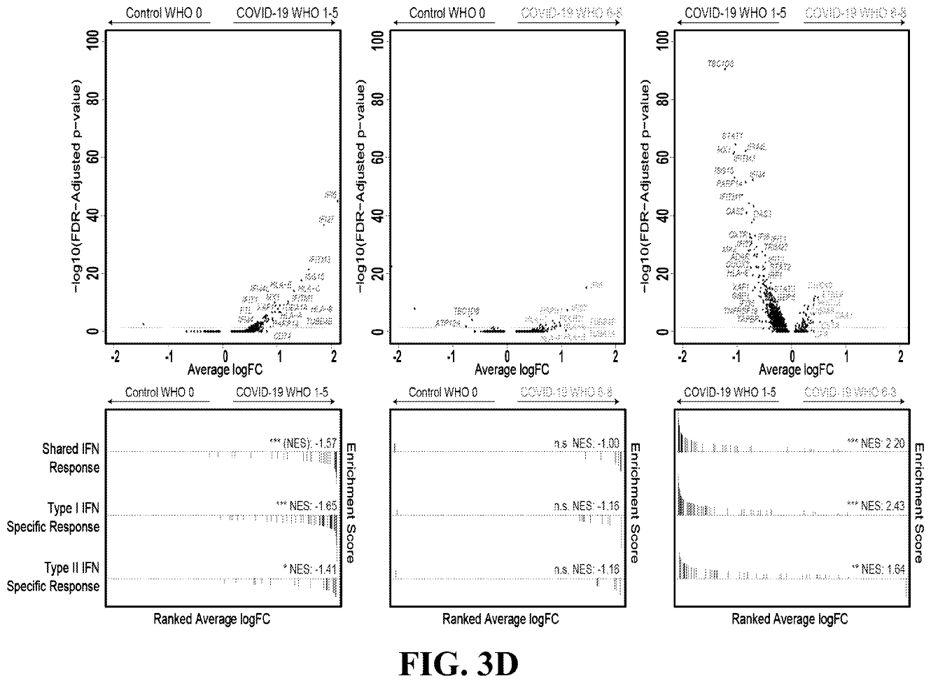

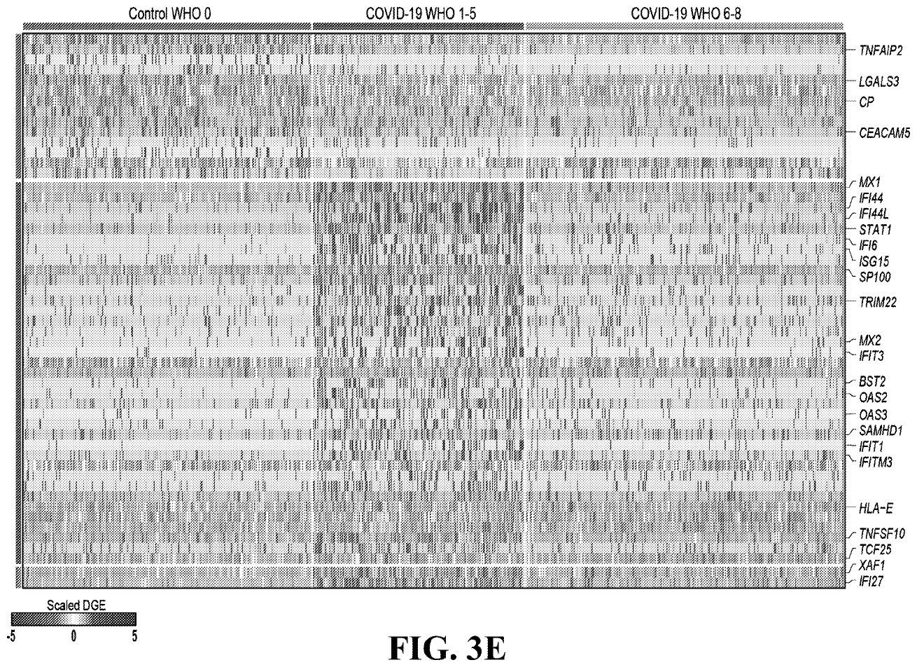

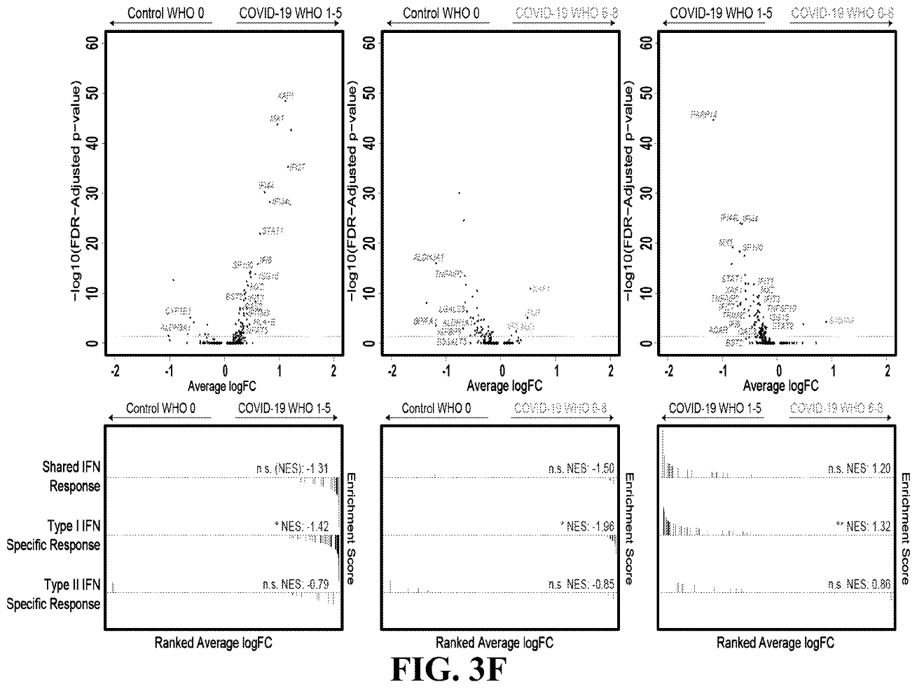

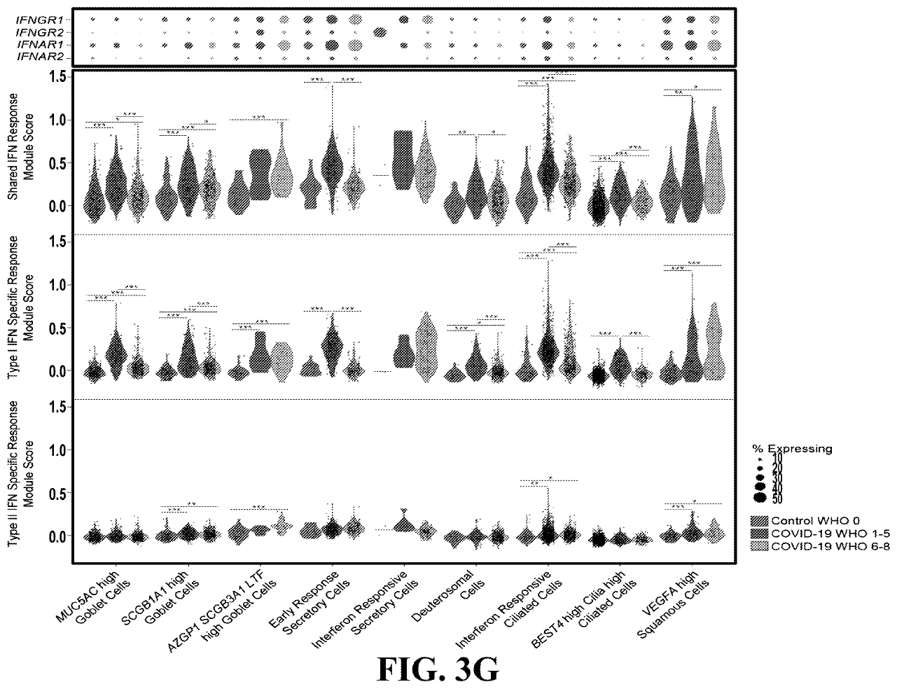

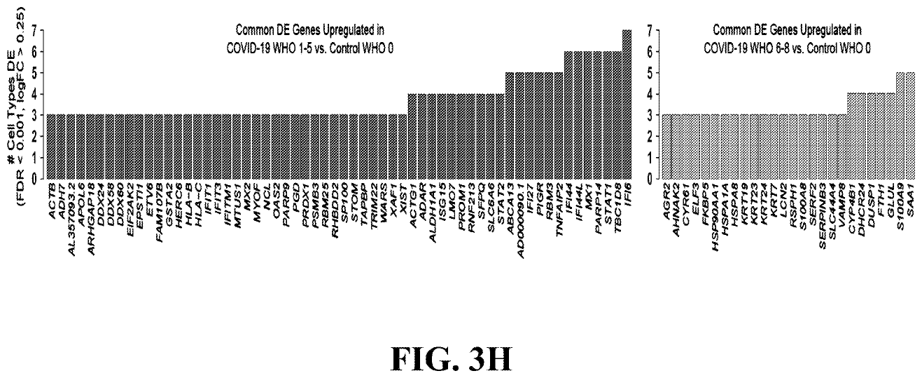

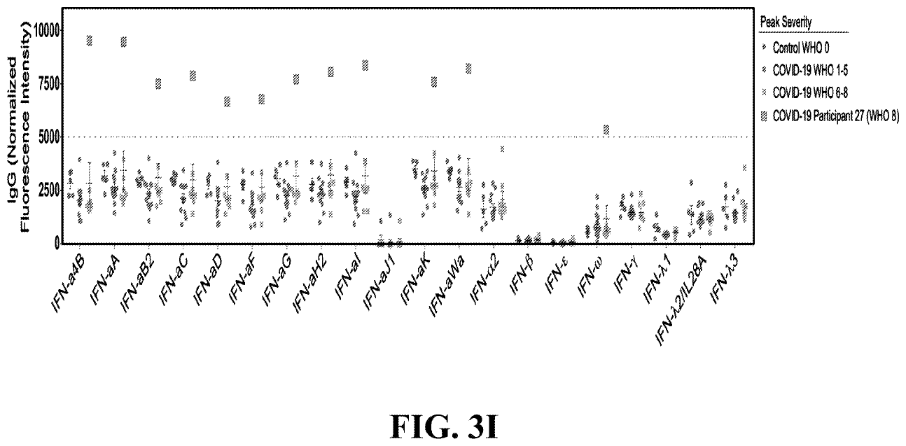

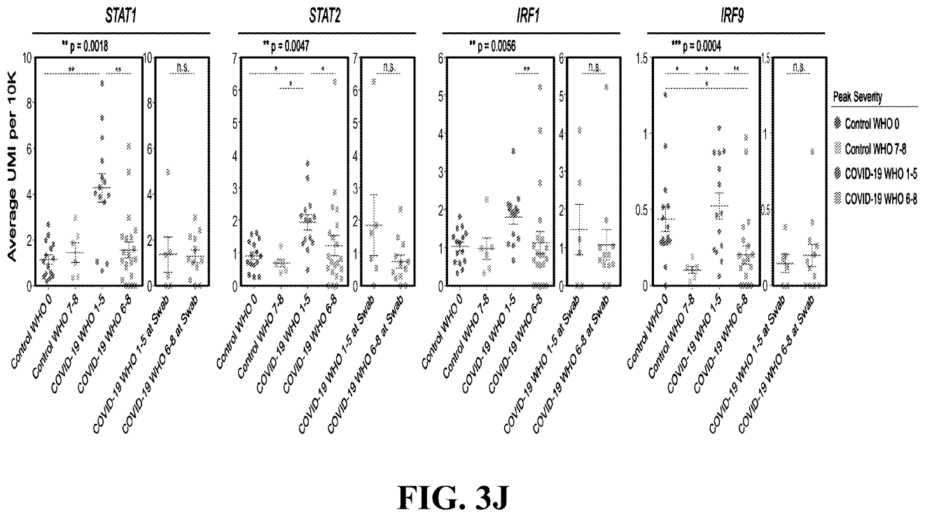

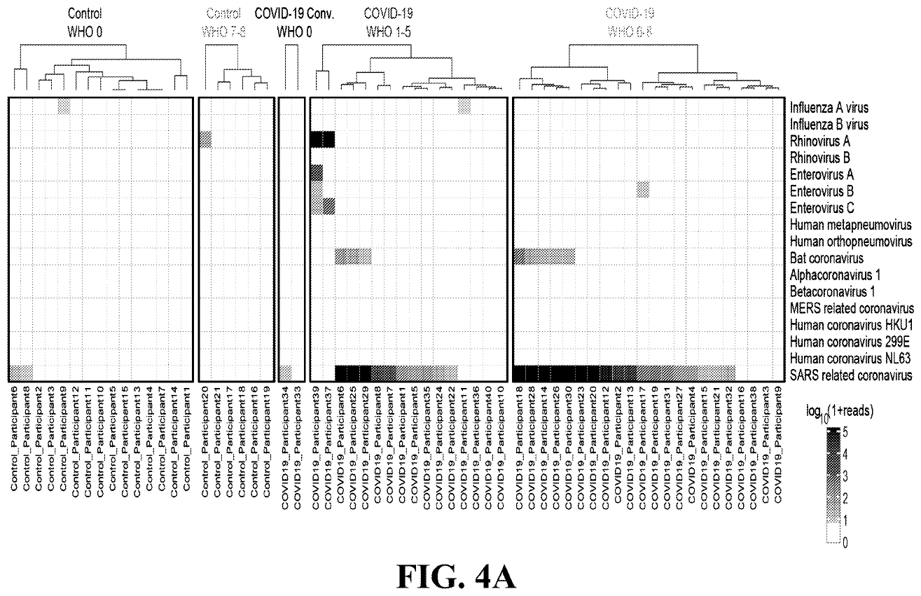

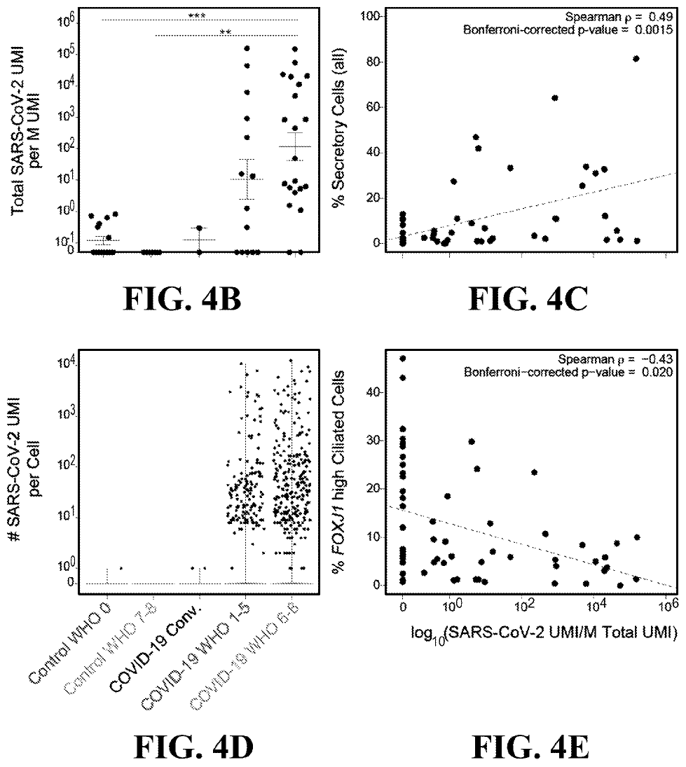

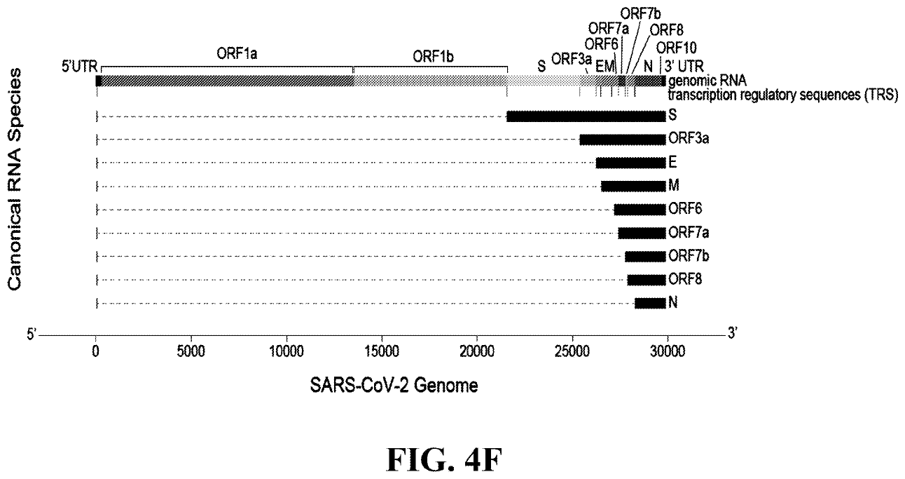

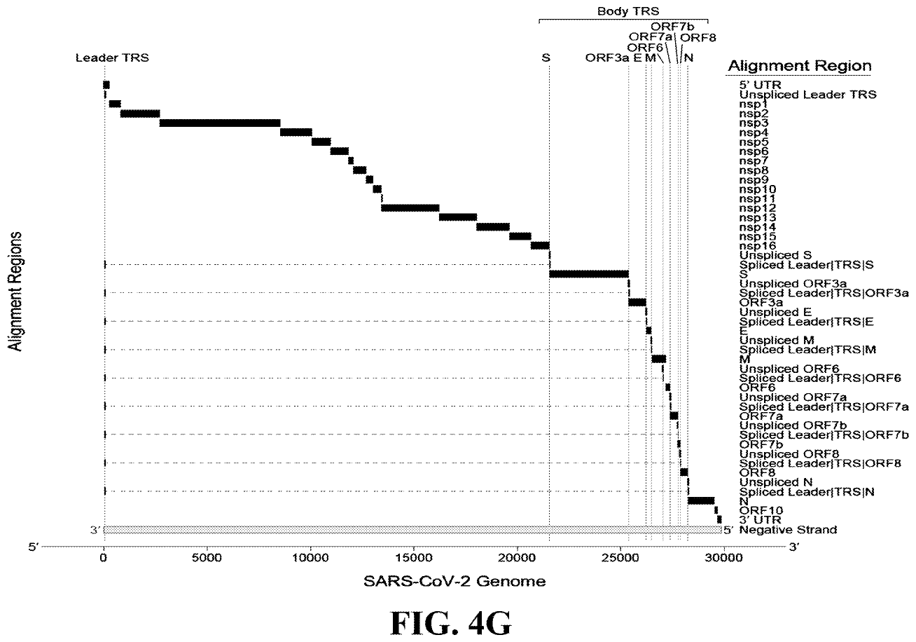

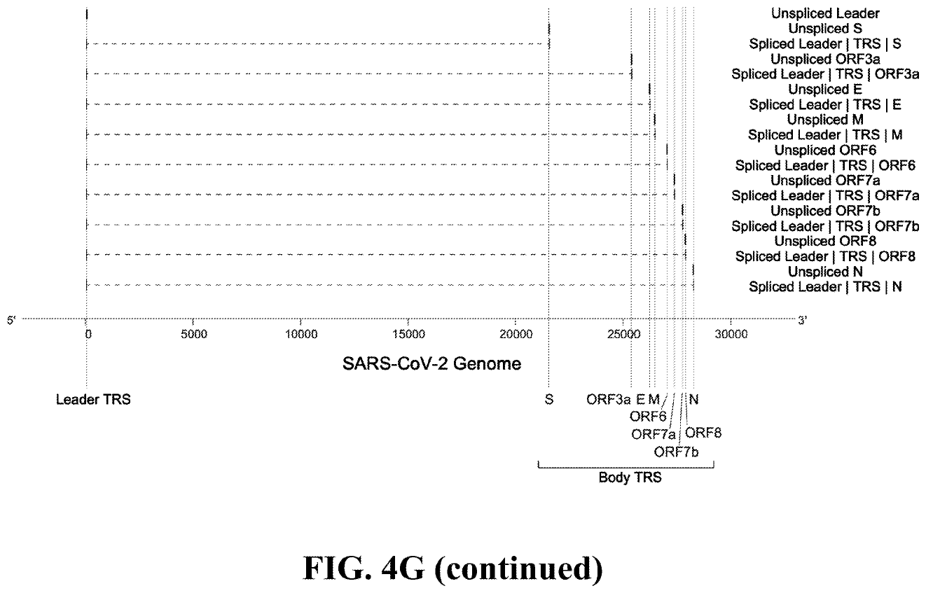

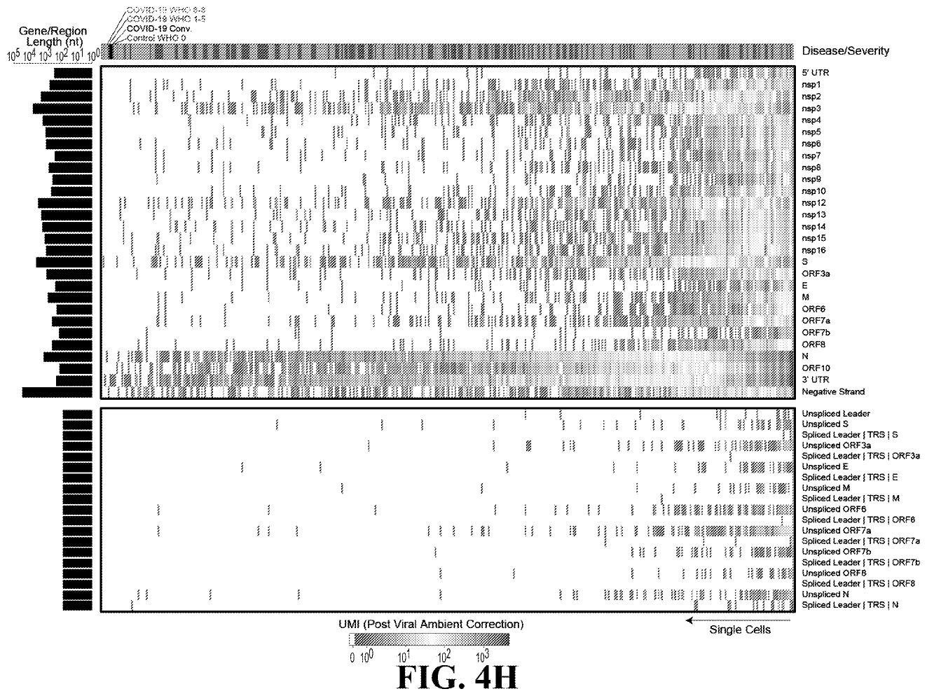

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sea sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333], which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A below shows several Cas polypeptides and the PAM sequence they recognize.

TABLE A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 | TTTV |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein HisA, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/naturel4592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 1)
M D P I R S R T P S P A R E L L S G P Q
P D G V Q P T A D R G V S P P A G G P L
D G L P A R R T M S R T R L P S P P A P
S P A F S A D S F S D L L R Q F D P S L
E N T S L F D S L P P F G A H H T E A A
T G E W D E V Q S G L R A A D A P P P T
M R V A V T A A R P P R A K P A P R R R
A A Q P S D A S P A A Q V D L R T L G Y
S Q Q Q Q E K I K P K V R S T V A Q H H
E A L V G H G F T H A H I V A L S Q H P
A A L G T V A V K Y Q D M I A A L P E A
T H E A I V G V G K Q W S G A R A L E A
L L T V A G E L R G P P L Q L D T G Q L
L K I A K R G G V T A V E A V H A W R N
A L T G A P L N

An exemplary amino acid sequence of a C-terminal capping region is:

(SEQ ID NO: 2)
R P A L E S I V A Q L S R P D P A L A A
L T N D H L V A L A C L G G R P A L D A
V K K G L P H A P A L I K R T N R R I P
E R T S H R V A D H A Q V V R V L G F F
Q C H S H P A Q A F D D A M T Q F G M S
R H G L L Q L F R R V G V T E L E A R S
G T L P P A S Q R W D R I L Q A S G M K
R A K P S P T S T Q T P D Q A S L H A F
A D S L E R D L D A P S P M H E G D Q T
R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include, but are not limited to, BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e., an activation domain), such as the VP16, VP64 or p65 activation domain. In some embodiments, the nucleic acid binding is linked, for example, with an effector domain that includes, but is not limited to, a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 3) or PKKKRKVEAS (SEQ ID NO: 4); the NLS from nucleoplasmin (e.g., the nucleoplasmin bipartite NLS with the sequence KRPAATKKAGQAKKKK (SEQ ID NO: 5)); the c-myc NLS having the amino acid sequence PAAKRVKLD (SEQ ID NO: 6) or RQRRNELKRSP (SEQ ID NO: 7); the hRNPA1 M9 NLS having the sequence NQSSNFGPMKGGNFGGRSSGPYGGGGQYFAKPRNQGGY (SEQ ID NO: 8); the sequence RMRIZFKNKGKDTAELRRRRVEVSVELRKAKKDEQILKRRNV (SEQ ID NO: 9) of the IBB domain from importin-alpha; the sequences VSRKRPRP (SEQ ID NO: 10) and PPKKARED (SEQ ID NO: 11) of the myoma T protein; the sequence PQPKKKPL (SEQ ID NO: 12) of human p53; the sequence SALIKKKKKMAP (SEQ ID NO: 13) of mouse c-abl IV; the sequences DRLRR (SEQ ID NO: 14) and PKQKKRK (SEQ ID NO: 15) of the influenza virus NS1; the sequence RKLKKKIKKL (SEQ ID NO: 16) of the Hepatitis virus delta antigen; the sequence REKKKFLKRR (SEQ ID NO: 17) of the mouse M×1 protein; the sequence KRKGDEVDGVDEVAKKKSKK (SEQ ID NO: 18) of the human poly(ADP-ribose) polymerase; and the sequence RKCLQAGMNLEARKTKK (SEQ ID NO: 19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively, or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 1 1, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA”, used interchangeably herein, are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Screening Methods

### Identifying Novel and Improved Treatments

In certain embodiments, the cell subset frequency and/or differential cell states (e.g., intrinsic immune response) can be detected for screening of novel therapeutic agents. In certain embodiments, the present invention can be used to identify improved treatments by monitoring the identified cell states in a subject undergoing an experimental treatment. In certain embodiments, an organoid system is used to detect shifts in the identified cell states to identify agents capable of shifting a subject from a severe disease state to a mild/moderate state (see, e.g., Yin X, Mead B E, Safaee H, Langer R, Karp J M, Levy O. Engineering Stem Cell Organoids. Cell Stem Cell. 2016; 18(1):25-38). As used herein, the term “organoid” or “epithelial organoid” refers to a cell cluster or aggregate that resembles an organ, or part of an organ, and possesses cell types relevant to that particular organ. Organoid systems have been described previously, for example, for brain, retinal, stomach, lung, thyroid, small intestine, colon, liver, kidney, pancreas, prostate, mammary gland, fallopian tube, taste buds, salivary glands, and esophagus (see, e.g., Clevers, Modeling Development and Disease with Organoids, Cell. 2016 Jun. 16; 165(7):1586-1597). In certain embodiments, a tissue system or tissue explant is used to detect shifts in the identified cell states to identify agents capable of shifting a subject from a severe disease state to a mild/moderate state (see, e.g., Grivel J C, Margolis L. Use of human tissue explants to study human infectious agents. Nat Protoc. 2009; 4(2):256-269). In certain embodiments, an animal model is used to detect shifts in the identified cell states to identify agents capable of shifting a subject from a severe disease state to a mild/moderate state (see, e.g., Munoz-Fontela C, Dowling W E, Funnell S G P, et al. Animal models for COVID-19. Nature. 2020; 586(7830):509-515).

In certain embodiments, candidate agents are screened. The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, the present invention provides for gene signature screening to identify agents that shift expression of the gene targets described herein (e.g., cell subset markers and differentially expressed genes). The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The gene signatures or biological programs of the present invention may be used to screen for drugs that reduce the signature or biological program in cells as described herein.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to identify small molecules capable of modulating a gene signature or biological program of the present invention in silico.

Further embodiments are illustrated in the following Examples which are given for illustrative purposes only and are not intended to limit the scope of the invention.

### EXAMPLES

### Example 1—Defining Cellular Diversity in the Human Nasopharynx (Nasopharyngeal Mucosa)

Here, Applicants present a comprehensive analysis of the cellular phenotypes in the nasal mucosa during early SARS-CoV-2 infection. To achieve this, Applicants developed tissue handling protocols that enabled high-quality scRNA-seq from frozen nasopharyngeal swabs collected from a large patient cohort (n=58) and created a detailed map of epithelial and immune cell diversity. Applicants found that SARS-CoV-2 infection leads to a dramatic loss of mature ciliated cells, which is associated with secretory cell expansion, differentiation, and the accumulation of deuterosomal cell intermediates—potentially involved in the compensatory repopulation of damaged ciliated epithelium. While Applicants observe broad induction of interferon-responsive and anti-viral genes in cells from individuals with mild/moderate COVID-19, severe COVID-19 is characterized by a dramatically blunted interferon response, and mucosal recruitment of highly inflammatory myeloid populations, which represent the primary sources of tissue pro-inflammatory cytokines including TNF, IL1B, and CXCL8. Further, using unbiased whole-transcriptomic amplification, Applicants map not only host cellular RNA, but also cell-associated SARS-CoV-2 RNA, allowing us to trace viral tropism to specific epithelial subsets and identify host pathways linked with susceptibility or resistance to viral infection. Together, the data suggest that an early failure of intrinsic anti-viral immunity among nasal epithelial cells responding to SARS-CoV-2 infection may underlie and predict progression to severe COVID-19.

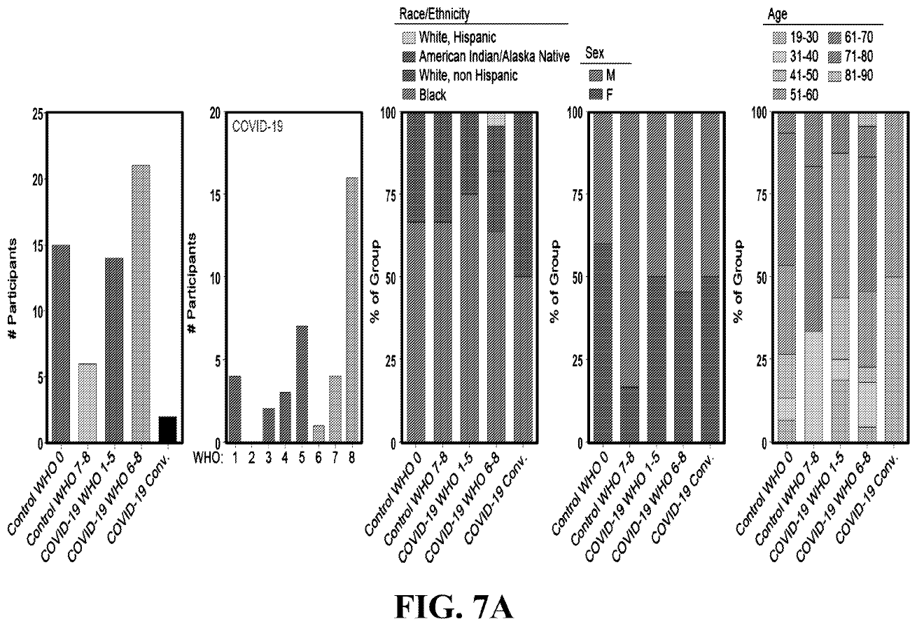

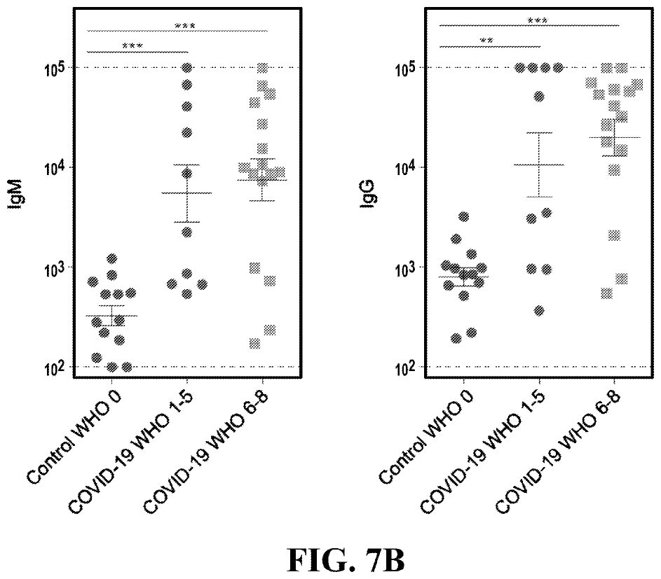

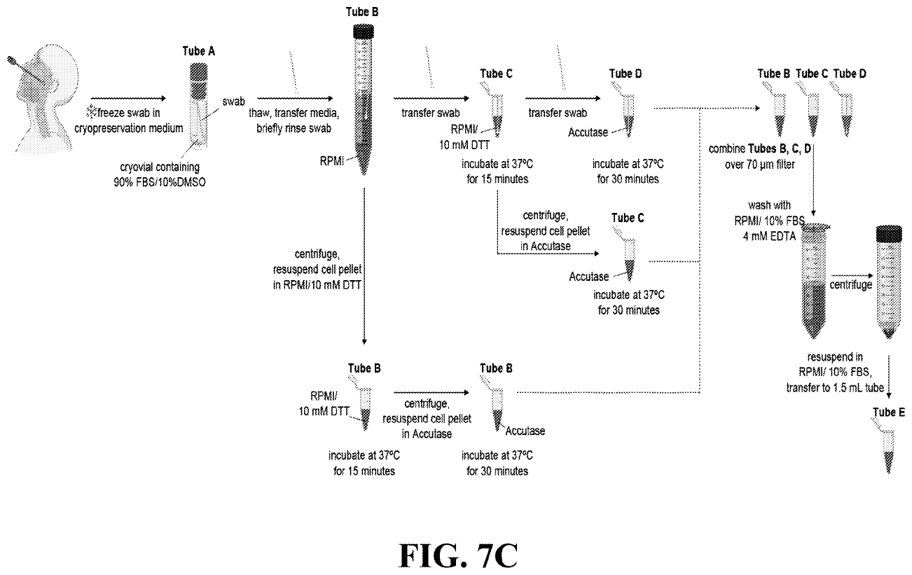

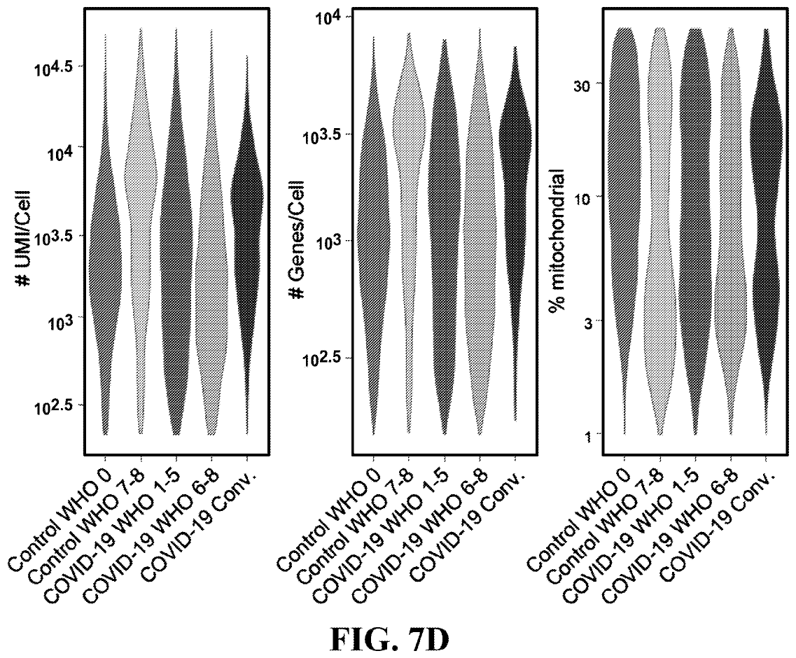

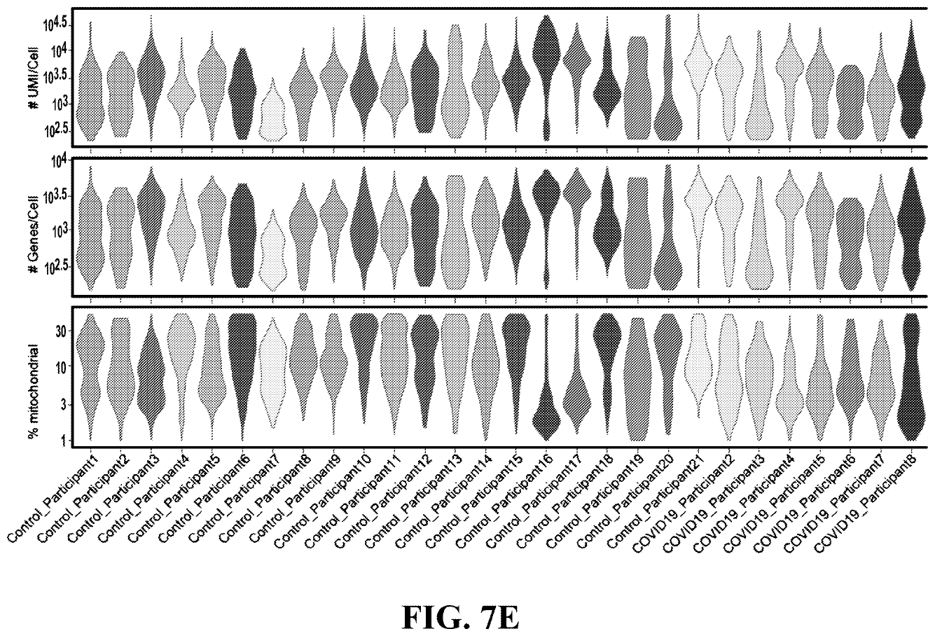

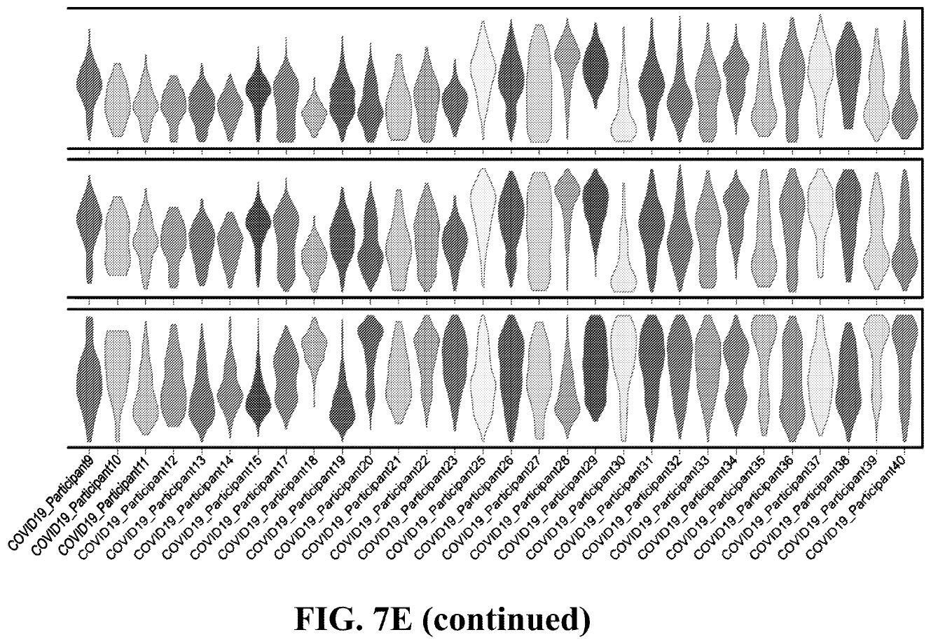

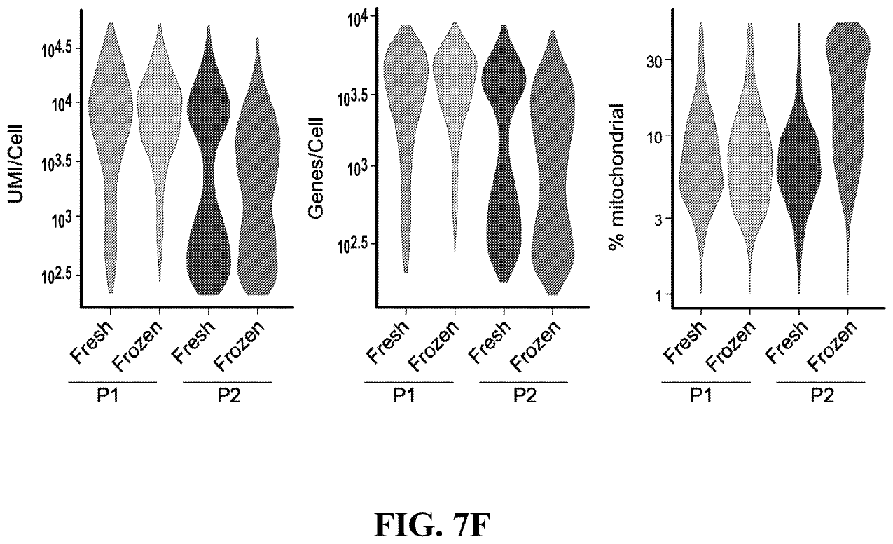

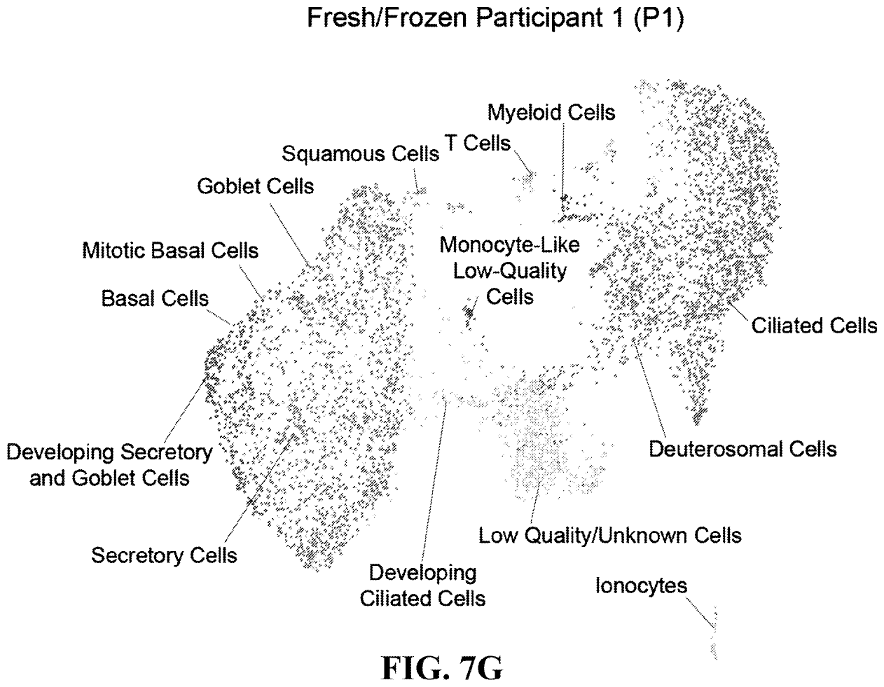

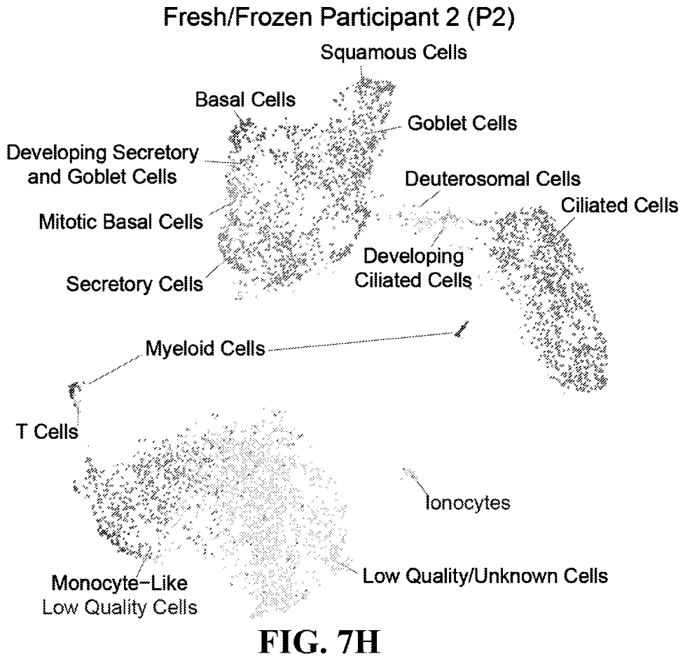

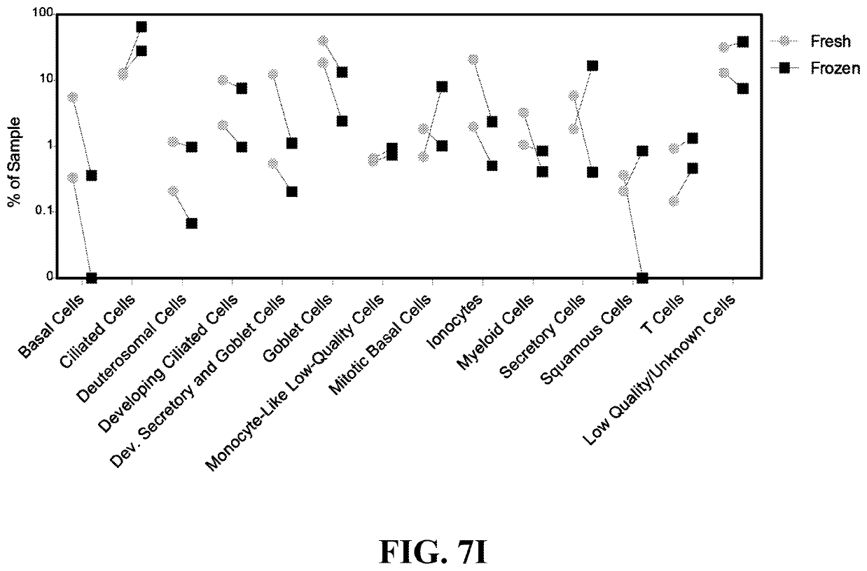

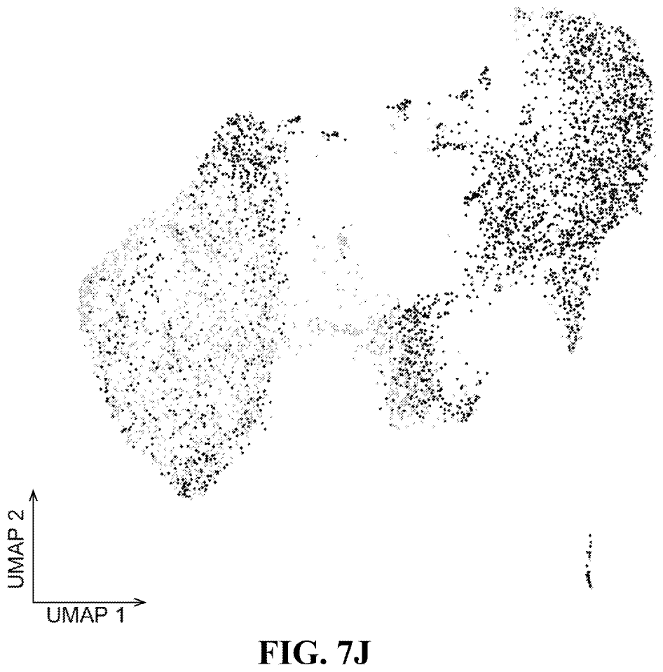

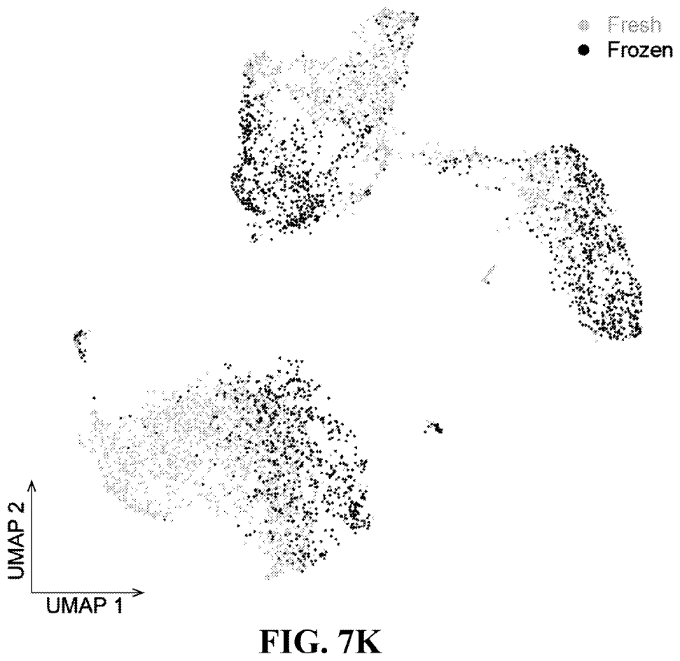

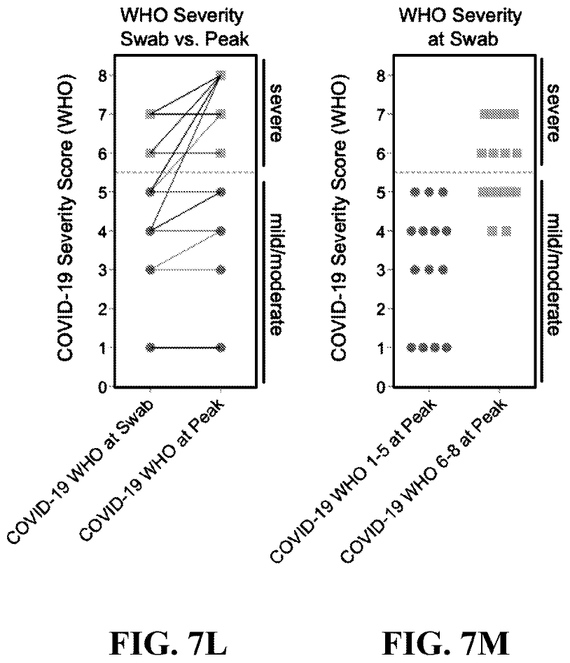

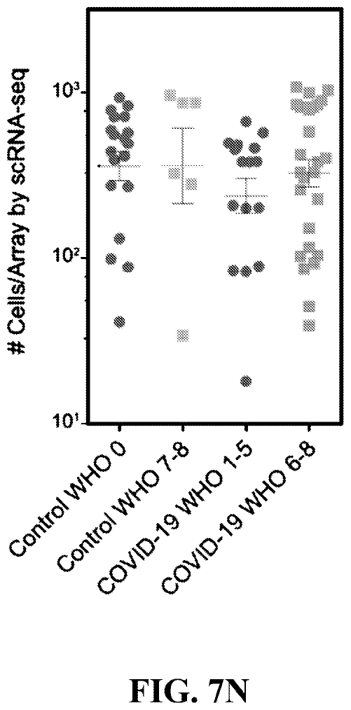

Nasopharyngeal (NP) swabs were collected from 58 individuals from the University of Mississippi Medical Center (UMMC) between April and September 2020. This cohort consisted of 35 individuals who had a positive SARS-CoV-2 PCR NP swab on the day of hospital presentation. A Control group consisted of 15 individuals who were asymptomatic and had a negative SARS-CoV-2 NP PCR, 6 intubated individuals in the intensive care unit without a recent history of COVID-19 and negative SARS-CoV-2 NP PCR, and 2 additional individuals with recent history of COVID-19 and negative SARS-CoV-2 NP PCR, classified as “Convalescent” (Table 6, see Methods for full inclusion and exclusion criteria). 38 individuals were diagnosed with COVID-19, and nasopharyngeal swabs were collected within the first 3 days following admission to the hospital. Using the World Health Organization (WHO) guidelines for stratification and classification of COVID-19 severity based on the level of required respiratory support, 16 of the individuals were considered COVID-19 mild/moderate (WHO score 1-5) and 22 had severe COVID-19 (WHO score 6-8) (see Methods, Table 6, FIGS. 7A, 7B for complete demographic and clinical information). Patient groups by WHO score reflects the peak disease severity, rather than the severity at the moment samples were collected. Applicants grouped individuals with COVID-19 based on the maximum (“peak”) level of required respiratory support (World Health Organization, 2020). Samples from the nasopharyngeal epithelium were taken by a trained healthcare provider and rapidly processed and cryopreserved to maintain cellular viability (FIG. 1A, FIG. 7C). Swabs were later processed to recover single-cell suspensions (mean+/−SEM: 57,000+/−15,000 total cells recovered per swab), before generating single-cell transcriptomes using the Seq-Well S^{3 44-46}.

Among all COVID-19 and Control samples, Applicants recovered 32,871 genes across 32,588 cells (following filtering and quality control), with an average recovery of 562+/−69 cells per swab (mean+/−SEM). Among recovered cells, Applicants found roughly equivalent transcriptomic quality following uniform preprocessing steps and filtering (see Methods) between COVID-19 and Control participants, despite high variability in cellular recovery and quality of recovered cells between participants (FIGS. 7D, 7E). Following dimensionality reduction and clustering approaches to resolve individual cell types and cell states, Applicants annotated 18 clusters corresponding to distinct cell types across immune and epithelial identities (FIG. 1B-E, Table 1). As tissue sampling relied on surface-resident cells that were gently scraped off of the nasopharyngeal epithelium, Applicants did not expect to recover stromal cell populations such as endothelial cells, fibroblasts, or pericytes, which were found in previous scRNA-seq datasets from nasal epithelial surgical samples^{47,48}. Among epithelial cell types, Applicants readily identified both Basal Cells by their expression of canonical marker genes including TP63, KRT15, KRT5, as well as Mitotic Basal Cells based on the added expression of genes involved in the cell cycle such as MKI67, and TOP2A (FIG. 1F). Applicants resolved large populations of both Secretory Cells and Goblet cells, identified by expression of KRT7, CXCL17, F3, AQP5, and CP. Despite strong transcriptional similarity between Secretory and Goblet cells, Applicants distinguished between both cell types based on expression of MUC5AC, which defines Goblet Cells, and BPIFA1, which Applicants found primarily expressed within Secretory Cell types and diminished in MUC5AC high cells. Applicants also designated a small population of cells Developing Secretory and Goblet Cells based on their lower expression of classic Secretory/Goblet Cell genes, as well as persistent expression of some Basal Cell markers (e.g., persistent COL7A1 and DST expression, but diminishing KRT5/KRT15 expression). Applicants also distinguished between goblet and secretory cells based on expression of MUC5AC-expressing goblet, and BPIFA1-expressing secretory cells. Applicants also resolved a population of ionocytes, a recently-identified specialized subtype of secretory cell present in respiratory epithelia defined by expression of transcription factors FOXI1 and FOXI2, as well as CTFR—thus thought to play a role in mucous viscosity^{49,51}. Squamous cells were identified by their expression of SCEL, as well as multiple SPRR− genes, and likely derive from pharyngeal/oral squamous cells as well those within the nasal epithelium. Applicants also recovered a very small population of cells Applicants term “Enteroendocrine Cells”, based on unique expression of gastric inhibitory polypeptide (GIP), which is typically produced by intestinal and gastric enteroendocrine cells and LGR5, which classically marks stem cell populations in the gastrointestinal mucosa.

Ciliated cells were the most numerous epithelial cell type recovered in this dataset, defined by expression of transcription factor FOXJJ as well as numerous genes involved in the formation of cilia, e.g., DLEC1, DNAH11, and CFAP43. Similar to intermediate/developing cells of the secretory and goblet lineage, Applicants also identified two populations of precursor ciliated cells. One, termed Developing Ciliated Cells, which expressed canonical Ciliated Cell genes such as FOXJJ, CAPSL, and PIFO, however lower than mature Ciliated Cells and without the expression of cilia-forming genes. Applicants also identified a cluster defined by expression of DEUP1, which is critical for centriole amplification as a precursor to cilium assembly. Together with co-expression of CCNO, CDC20, FOXN4, and HES6, these cells match a recently-defined cell type termed Deuterosomal Cells⁴⁸, which represent an intermediate cell type in which Secretory cells trans-differentiate into Ciliated Cells.

Immune cells represent a minority of recovered cells, yet Applicants resolved multiple distinct clusters and cell types, representing major myeloid and lymphoid populations. Among lymphoid cells, Applicants recovered T cells, identified by CD3E, CD2, TRBC2 expression, and B cells, identified by MS4A1, CD79A, CD79B expression. Among myeloid cell types, Applicants recovered a large population of Macrophages (CD14, FCGR3A, VCAN), Dendritic Cells (CCR7, CD86), and Plasmacytoid DCs (IRF7, IL3RA). Relative to true tissue-resident abundances, Applicants under-recovered granulocyte populations, likely due to the intrinsic fragility of these cell types and the cryopreservation methods required in the sample pipeline. Applicants recovered a very small population of Mast Cells, defined by expression of GATA2, TPSB2, and PTGS2. Among two samples, Applicants recovered Erythroblast-like cells, defined by expression of hemoglobin subunits including HBB and HBA2. With the exception of Erythroblasts, each cell type was represented by cells from numerous participants, and from each participant Applicants recovered a diversity of cell types and states, though the cellular composition was highly variable between distinct individuals (FIG. 1G, 1H).

Applicants directly tested whether cell types collected from nasal swabs following cryopreservation were representative of cellular composition extracted from a freshly swabbed nasal epithelium, or if certain cell types were lost during freezing (FIG. 7F-7K). Recovery of viable cells, technical metrics of single-cell library quality, and cellular proportions after clustering and analysis were all largely stable between matched fresh and cryopreserved swabs taken from the same individual. Importantly, no “new” cell types were recovered from the freshly processed samples (from healthy participants), thus supporting adequate data representation of the nasal mucosa even following on-swab cryopreservation.

Applicants interrogated each cell type for their expression of host factors utilized by common respiratory viruses for cellular entry (FIG. 1I)^{35,51-55}. Applicants found ACE2 expression highest among Secretory Cells and Goblet Cells, and to a lesser extent on Ciliated Cells, Developing Ciliated Cells, Deuterosomal Cells, and Squamous Cells—suggesting these cells are likely targets for SARS-CoV-2 (and other beta coronaviruses that use ACE2 as their primary cellular entry factor). SARS-CoV-2 spike protein requires “priming” or cleavage by host proteases to enable membrane fusion and viral release into the cell, since early 2020, researchers have identified TMPRSS2, TMPRSS4, CTSL, and FURIN as capable of spike protein cleavage and critical for viral entry⁵¹. TMPRSS2, thought to be the principal host factor for SARS-CoV-2 S cleavage, is found in highest abundance on Squamous Cells, followed by modest expression on all other epithelial cell types. Similarly, CTSL (and other cathepsins) was found across diverse epithelial and myeloid cell types. ANPEP and DPP4, host receptors targeted by other Human coronaviruses causing upper respiratory diseases, are found primarily on Goblet Cells and Secretory Cells. As expected, CDHR3, the receptor utilized by Rhinovirus C, is found primarily on Ciliated Cells and Developing Ciliated Cells.

Next, Applicants binned both Control and COVID-19 participants by their level of respiratory support according to the WHO scoring system: Control WHO 0 (comprising healthy SARS-CoV-2 PCR negative participants, n=15), Control WHO 7-8 (SARS-CoV-2 PCR negative, incubated participants treated in the ICU for non-COVID-19 diagnoses, n=6), COVID-19 WHO 1-5 (SARS-CoV-2 PCR positive, mild/moderate disease, n=14), and COVID-19 WHO 6-8 (SARS-CoV-2 PCR positive, intubated, severe disease, n=21). Applicants compared proportional cell type abundances from the coarse cell type annotations across these four disease cohorts (FIG. 1J-1N). Applicants found that the abundance of Ciliated Cells (all, coarse annotation) was significantly impacted by cohort (Bonferroni-corrected p=0.025) and were significantly reduced among COVID-19 WHO 6-8 participants compared to healthy controls (mean+/−SEM 17.1+/−3.6% of COVID-19 WHO 6-8 samples were Ciliated Cells, compared to 46.7+/−7.4% of Control WHO 0, p<0.01) (FIG. 1N). Deuterosomal cells, which represent a developmental intermediate as secretory/goblet cells trans-differentiate into ciliated cells, were significantly increased among Control WHO 7-8, COVID-19 WHO 1-5, and COVID-19 WHO 6-8 samples, with the strongest increases observed from participants with severe COVID-19 compared to healthy controls (FIG. 1L). Likewise, Developing Ciliated Cells were significantly increased among participants with severe COVID-19 (FIG. 1M). Secretory cells were also dramatically increased among all COVID-19 participants compared to non-COVID-19 controls, with 20.4+/−5.0% (mean+/−SEM) of all epithelial cells were Secretory Cells within severe COVID-19 participants, while mild/moderate COVID-19 participants contained 8.3+/−2.8% Secretory Cells, and on average, fewer than 4% of cells per participant were Secretory among either Control WHO 0 and Control WHO 7-8 samples (FIG. 1K). Goblet Cells, however, did not reach significance but were substantially increased in a subset of participants from the COVID-19 mild/moderate and severe groups (FIG. 1J). Intriguingly, expansion of secretory cells and loss of ciliated cells resulted in a net gain in epithelial diversity, calculated by Simpson's index which calculates the richness of the epithelial “ecosystem” (FIG. 1O).

### Example 2—Epithelial Diversity and Remodeling Following SARS-CoV-2 Infection

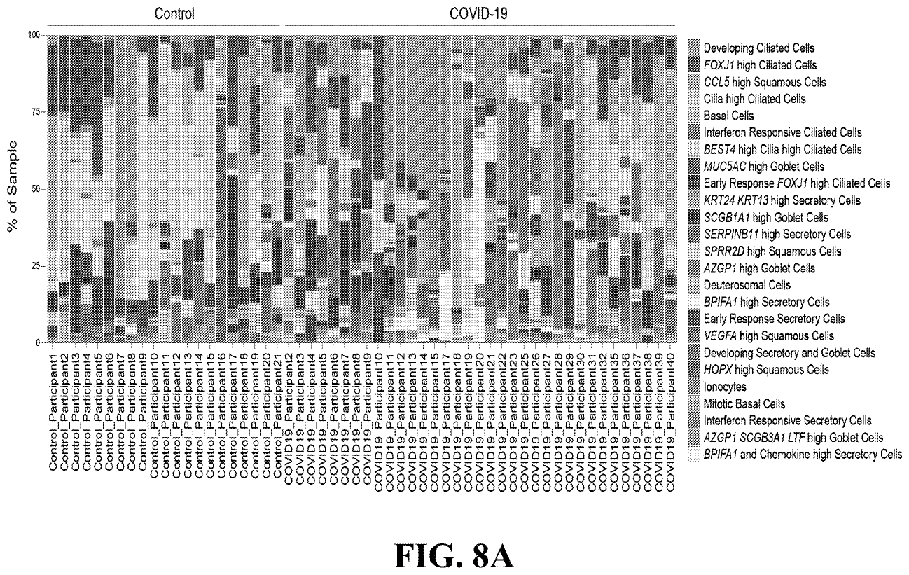

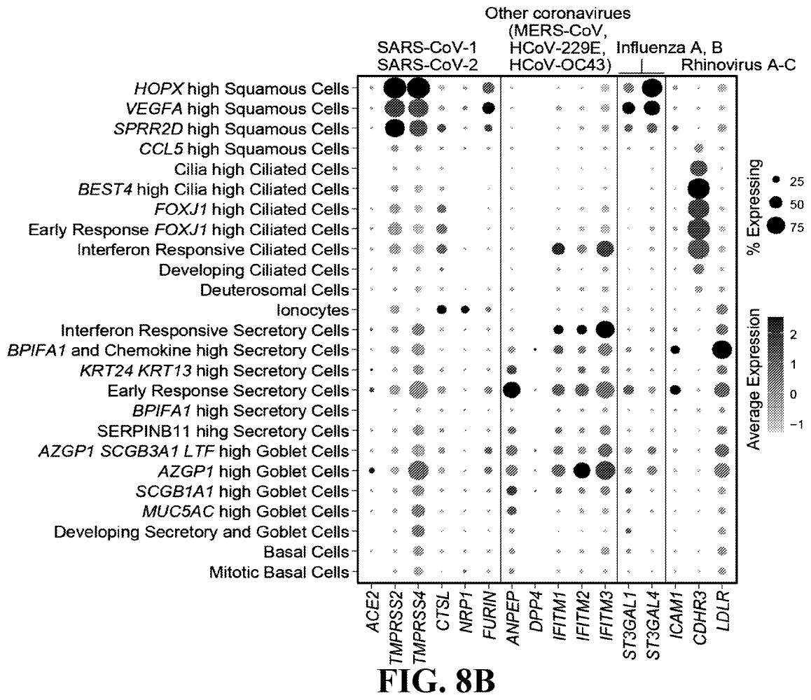

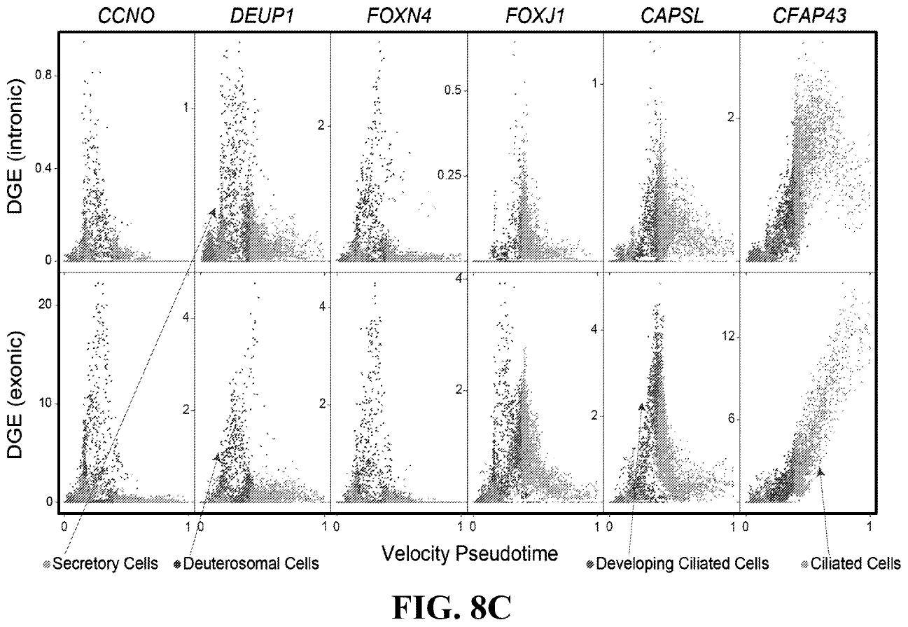

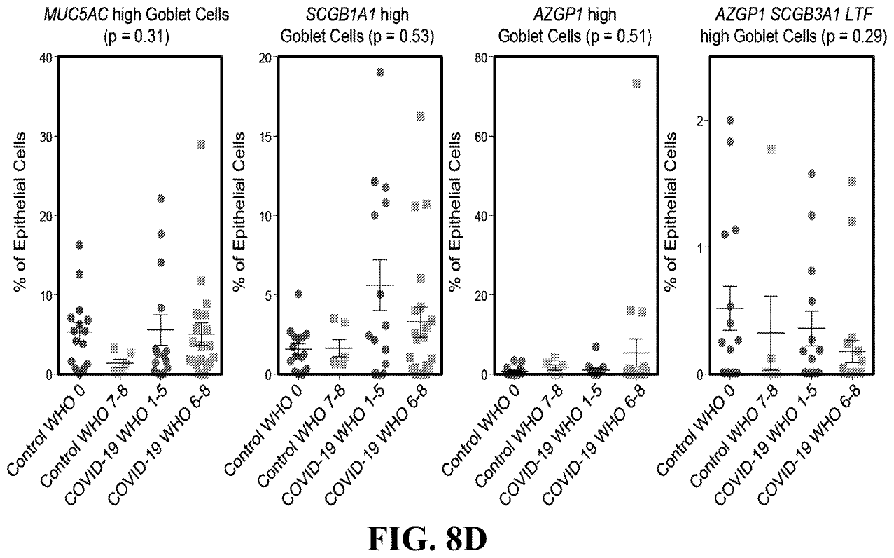

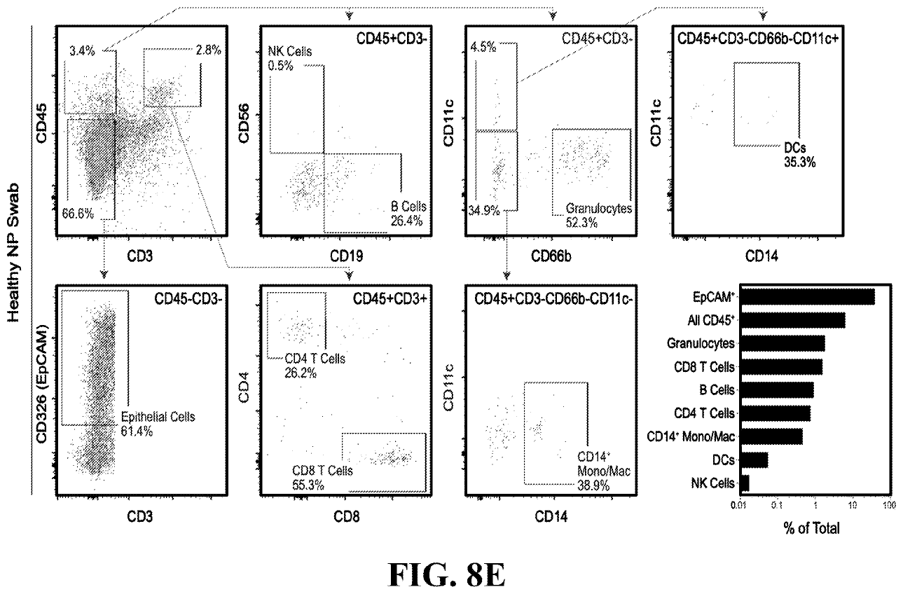

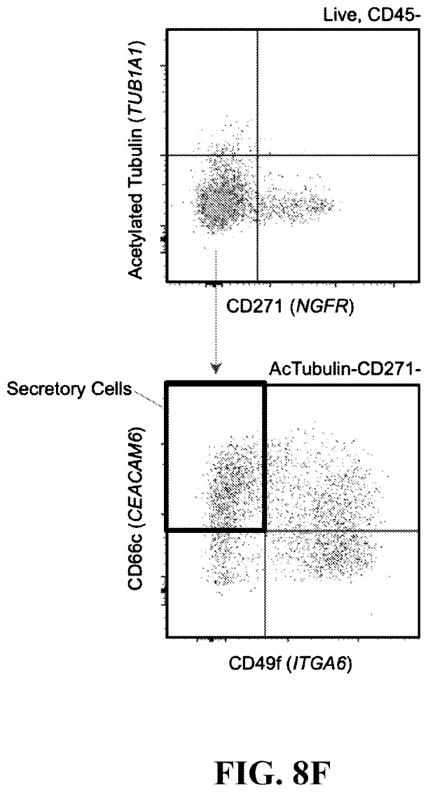

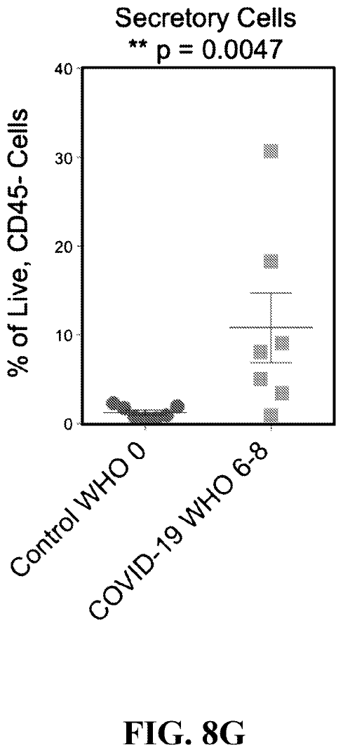

Next, Applicants sought to more completely delineate the diversity of epithelial cells through iterative clustering and sub-clustering among epithelial cell types (see Methods). This enabled Applicants to divide the 10 “Coarse” epithelial cell types into 25 “Detailed” cell types/states (FIG. 2A-2E, FIG. 8A, Table 1). Among some cell types, Applicants did not find additional within-type diversity, and thus the “Coarse” annotations (FIG. 2A) are equivalent to the “Detailed” identities (FIG. 2D). This applied to Ionocytes, Deuterosomal Cells, Developing Secretory and Goblet Cells, Basal Cells, Mitotic Basal Cells, and Developing Ciliated Cells. Applicants split Goblet Cells (Coarse annotation) into 4 distinct Detailed annotations: MUC5AC high Goblet Cells, which lacked additional specialized markers beyond classic Goblet Cell identifiers, SCGB1A1 high Goblet Cells, AZGP1 high Goblet Cells, and AZGP1 SCGB3A1 LTF high Goblet Cells (each named by a representative defining marker or marker set). Secretory Cells were divided into 6 distinct Detailed subtypes: SERPINB11 high Secretory Cells (which, similar to MUC5AC high Goblet Cells, represented a more “generic” Secretory Cell phenotype), BPIFA1 high Secretory Cells, Early Response Secretory Cells (which expressed genes such as JUN, EGR1, FOS, NR4A1), KRT24 KRT13 high Secretory Cells (which are highly similar to previously-described KRT13+ “hillock” cells), BPIFA1 and Chemokine high Secretory Cells (example chemokines include CXCL8, CXCL2, CXCL1, and CXCL3), and Interferon Responsive Secretory Cells (defined by higher expression of broad anti-viral genes including IFITM3, IFI6, and MX1). Subsets of Squamous Cells were also found—detailed Squamous Cell subtypes include CCL5 high Squamous Cells, VEGFA high Squamous cells (which express multiple vascular endothelial genes including VEGFA and VWF), SPRR2D high Squamous Cells (which, in addition to SPRR2D, express the highest abundances of multiple SPRR− genes including SPRR2A, SPRR1B, SPRR2E, and SPRR3), and HOPXhigh Squamous Cells. Finally, Ciliated Cells could be further divided into 5 distinct subtypes: Interferon Responsive Ciliated Cells (expressing anti-viral genes similar to other “Interferon Responsive” subsets, such as IFIT1, IFIT3, IFI6), FOXJ1 high Ciliated Cells, Early Response FOXJ1 high Ciliated Cells (which, in addition to high FOXJJ, also express higher abundances of genes such as JUN, EGR1, FOS than other ciliated cell subtypes), Cilia high Ciliated Cells (which broadly express the highest abundances of structural cilia genes, such as DLEC1 and CFAP100), and BEST4 high Cilia high Ciliated Cells (in addition to cilia components, also express the ion channel BEST4).

Here, Applicants again examined the epithelial subtypes for their expression of host entry factors which facilitate viral entry among common upper respiratory pathogens (FIG. 8B). ACE2 was previously identified as highest among Secretory, Goblet, and Ciliated Cells^{35,36}—here Applicants observe substantial within-cell type heterogeneity in ACE2 expression among each of these cell types. Notably, among Goblet cells, AZGP1 high Goblet Cells express the highest abundance of ACE2 mRNA, suggesting this cell type may be a preferential target for SARS-CoV-2 infection. Likewise, Early Response Secretory Cells, KRT24 KRT13 high Secretory Cells, and Interferon Responsive Secretory cells, all express elevated abundances of ACE2, and many other Secretory and Goblet Cell types express detectable ACE2, but lower levels. Similarly, multiple detailed subsets of Ciliated Cells expressed ACE2, however Cilia high and BEST4 high Cilia high Ciliated Cells notably did not appear to be actively transcribing ACE2 mRNA.

To map the differentiation trajectories and lineage relationships between epithelial cell types, Applicants analyzed single-cell RNA velocity (scVelo) across all epithelial cells. RNA velocity analysis leverages the dynamic relationships between expression of unspliced (intron-containing) and spliced (exonic) RNA across thousands of variable genes, enabling 1) estimation of the directionality of transitions between distinct cells and cell types, and 2) identification of putative driver genes behind these transitions. Overlaying the UMAPs of cell type identities and associated metadata in FIGS. 2A-2D, vector fields (black lines and arrows) represent a smoothed estimate of cellular transitions based on RNA velocity. Globally, RNA velocity appropriately places Basal Cells and Mitotic Basal Cells as the “root” or “origin” of cellular transitions, which then progress through the Developing Secretory and Goblet Cells to the Secretory Cells and Goblet Cells. Applicants hypothesize that the squamous cells recovered in this dataset arise from a distinct set of basal cells present in oral/upper esophageal mucosa, therefore their differentiation intermediates and trajectory are poorly represented here. Likewise, Applicants do not recover intermediate cell types for Ionocytes, so cannot trace their development from basal cells. Developing Ciliated Cells and Ciliated Cells are placed “later” in the differentiation trajectory, distal to development of both Secretory and Deuterosomal Cells, which is consistent with current models where ciliated cells represent a terminally differentiated state and may arise from these precursor cell types. By visualizing spliced and unspliced forms of representative markers underlying ciliated cell development, Applicants can visualize the transition from precursor Secretory Cell to Deuterosomal Cells to Developing Ciliated Cells, and finally mature Ciliated Cells differentiation (FIG. 8C).

Applicants next mapped and visualized developmental transitions and relationships between Basal, Goblet, and Secretory cell subtypes from the detailed cluster annotations (FIG. 2F-2I). As observed when considering all epithelial cells (FIG. 2A), Basal Cells and Mitotic Basal Cells were accurately predicted to represent the “root” of this differentiation trajectory. From here, TP63, KRT5 and LGR6 expression gradually decline across Basal and Developing Secretory and Goblet Cells, while expression of Secretory and Goblet Cell specific markers such as KRT7 and AQP5 progressively increase. The transition from Basal to Secretory and Goblet cell types through Developing Secretory and Goblet Cells is marked by transient upregulation of FGFR3 and progressive downregulation of EGFR. Notably, transitions between detailed Secretory and detailed Goblet cells are substantially less linear than among the coarse cell types or as seen in ciliated cells. RNA velocity curves predict multiple routes for development between distinct subtypes. This observation is consistent with the current understanding of respiratory secretory cell plasticity and capacity for de-differentiation.

Ciliated Cell subtypes were analyzed by their RNA velocity and pseudotemporal ordering in the same manner. Here, a focused UMAP with only Developing Ciliated Cells and Ciliated Cells is presented and overlaid with vector fields representing RNA velocity transitions (FIGS. 2J-2M). The velocity pseudotime predicts progression from Developing Ciliated Cells, to FOXJJ high Ciliated Cells, to BEST4 high Cilia high Ciliated Cells, and terminating in Cilia high Ciliated Cells. (FIG. 2M). Interferon Responsive Ciliated Cells and Early Response FOXJJ high Ciliated Cells represent phenotypic deviations from this ordered progression, and therefore appear collapsed/unresolved along this trajectory with the same pseudotime range as FOXJJ high Ciliated Cells.

Applicants next connected the composition of the detailed nasal epithelial microenvironment to the disease status of the participant (FIGS. 2N-2Q). Applicants mapped epithelial cell diversity and differentiation trajectories as before, including either cells from SARS-CoV-2 negative participants (FIG. 2P) or cells from SARS-CoV-2 positive participants (FIG. 2Q). Notably, cells from Control participants poorly populated the intermediate regions that bridge Secretory and Goblet Cell types to mature Ciliated Cells. Conversely, regions annotated as multiple Secretory Cell subsets and Developing Ciliated Cells were uniquely captured from COVID-19 participants. Dysregulated abundances of mature ciliated cell subsets were also observed, with decreased proportions of both Cilia high and BEST4 high Cilia high Ciliated Cells (representing the most terminally differentiated branches of ciliated cell development) among COVID-19 participants compared to healthy controls (FIG. 2O). Interferon Responsive Ciliated Cells were substantially increased among COVID-19 participants—averaging 15.9% of all epithelial cells among mild/moderate COVID-19 participants, compared to fewer than 1% among healthy controls. Among Secretory cell subtypes, BPIFA1 high Secretory cells were significantly elevated among participants with severe COVID-19, as were KRT13 KRT24 high Secretory Cells (FIG. 2N). Goblet Cells, Ionocytes, and Squamous Cells were largely unchanged by cohort, however SCGB1A1 high Goblet Cells were modestly increased among both mild/moderate and severe COVID-19 participants (FIG. 8D).

Together, the analysis defines both the cellular diversity among cells collected from nasopharyngeal swabs, as well as the nuanced developmental relationships between epithelial cells of the upper airway. Further, Applicants observe substantial expansion of immature/intermediate and specialized subtypes of secretory, goblet, and ciliated cells during COVID-19, presumably as a result of direct viral targeting and pathology, as well as part of the intrinsic capacity of the nasal epithelium to regenerate and repopulate following damage.

### Example 3—Alterations to Nasal Mucosal Immune Populations in COVID-19

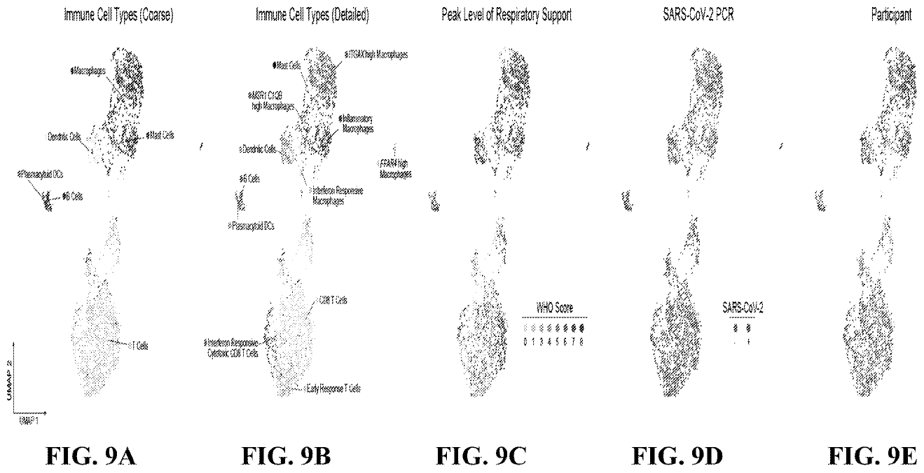

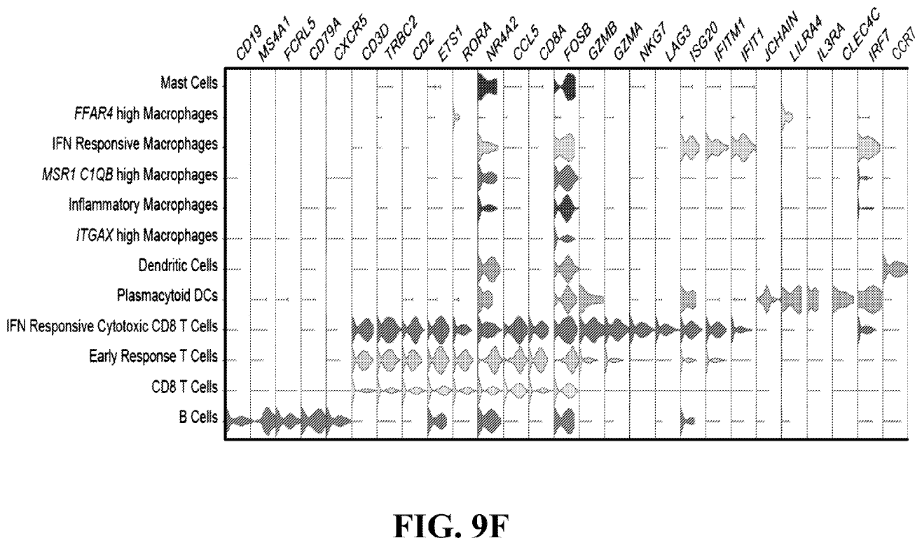

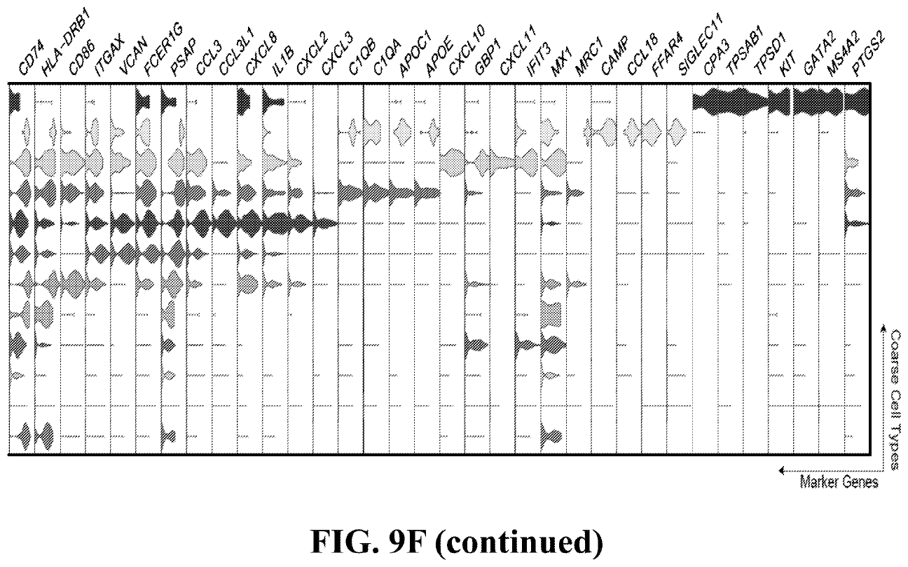

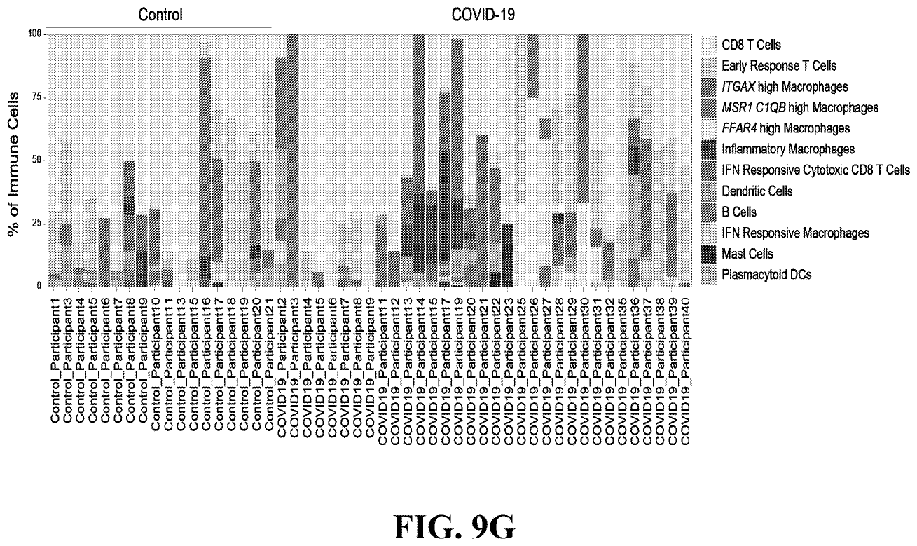

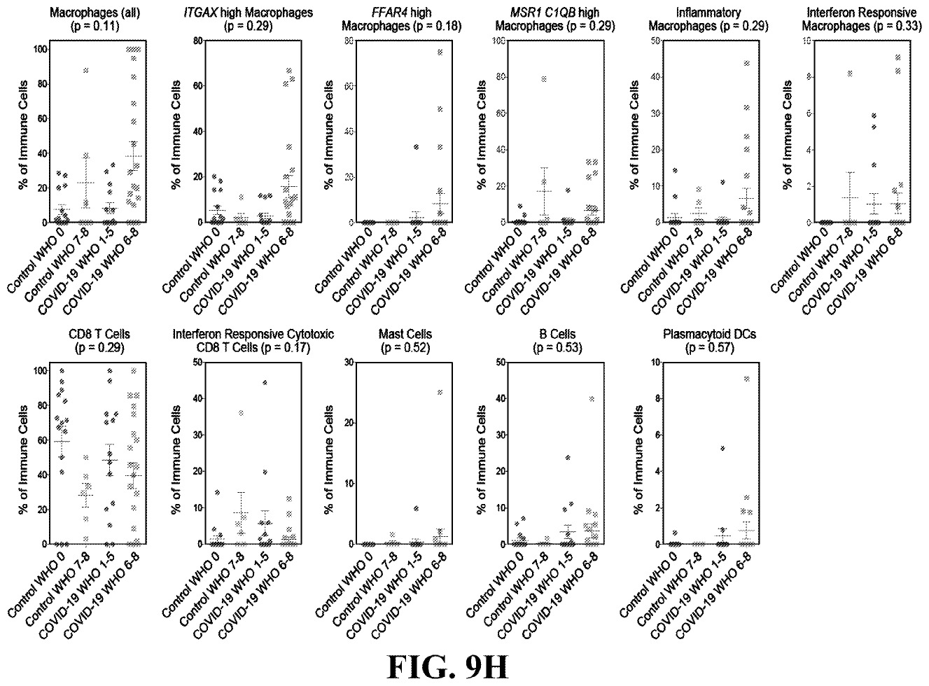

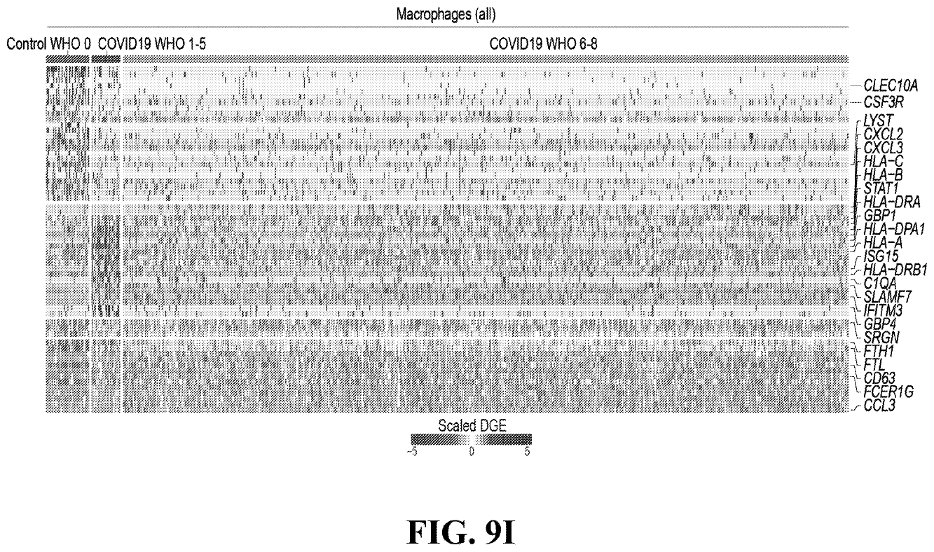

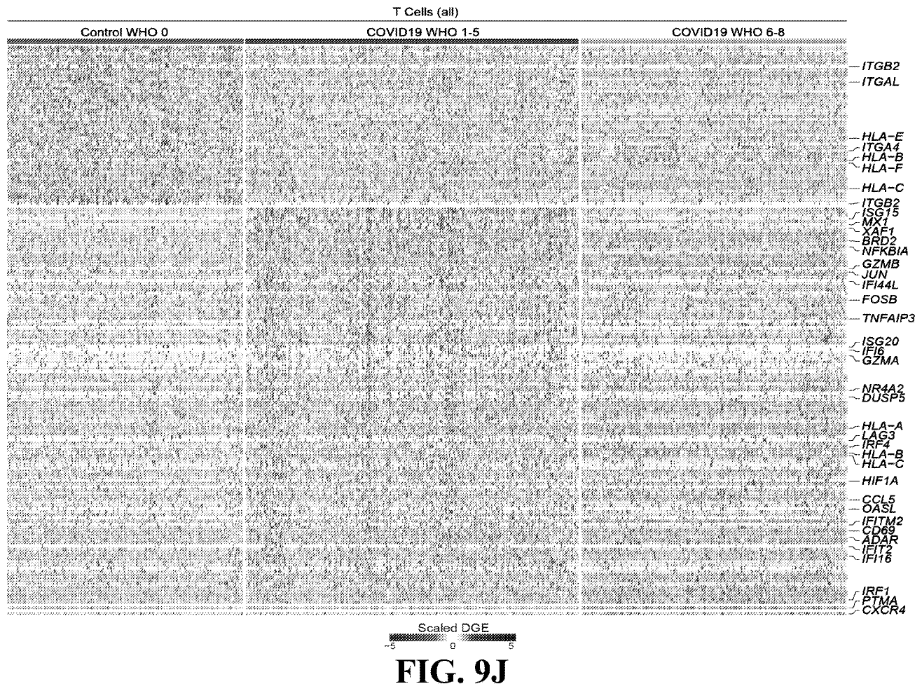

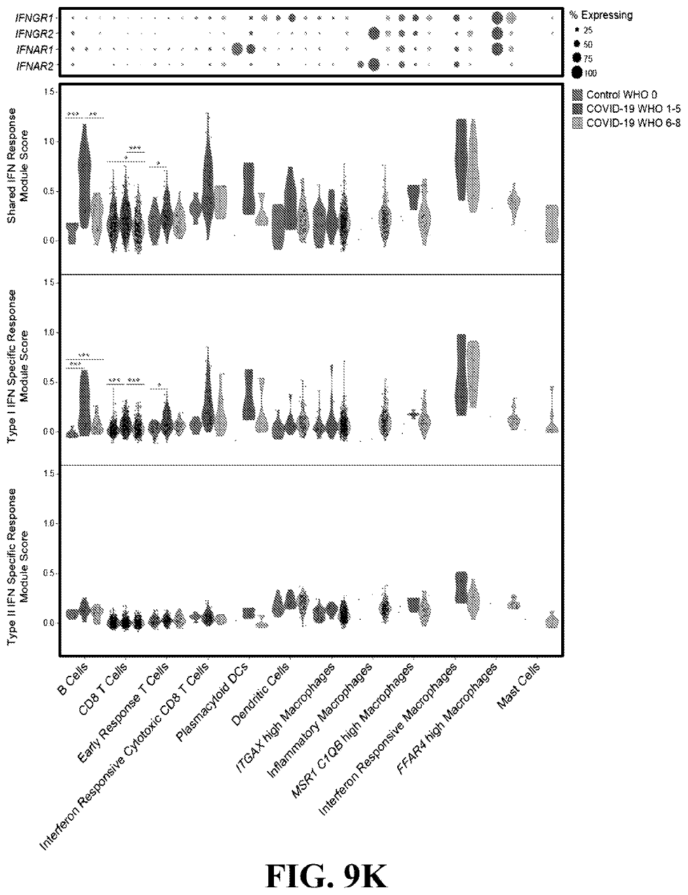

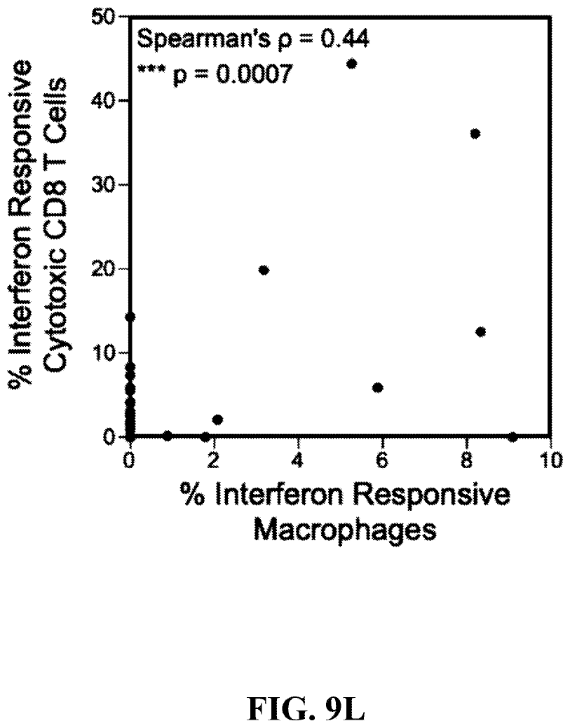

As with epithelial cells, Applicants further clustered and annotated detailed immune cell populations. Multiple cell types could not be further subdivided from their coarse annotation (FIG. 1B, FIG. 9A-9E), including Mast Cells, Plasmacytoid DCs, B Cells, and Dendritic Cells. Among Macrophages (coarse annotation), Applicants resolved 5 distinct subtypes (FIG. 9B). FFAR4 high Macrophages were defined by expression of FFAR4, MRC1, CHIT1, and SIGLEC11, as well as chemotactic factors including CCL18, CCL15, genes involved in leukotriene synthesis (ALOX5, ALOX5AP, LTA4H), and toll-like receptors TLR8 and TLR2 (Table 1, FIG. 9F). Interferon Responsive Macrophages were distinguished by elevated expression of anti-viral genes such as IFIT3, IFIT2, ISG15, and MX1, akin to the epithelial subsets labeled “Interferon Responsive”, along with CXCL9, CXCL10, CXCL11, which are likely indicative of IFNγ stimulation. MSR1 C1QB high Macrophages are defined by cathepsin expression (CTSD, CTSL, CTSB) and elevated expression of complement (C1QB, C1QA, C1QC), and lipid binding proteins (APOE, APOC, and NPC2). The fourth “specialized” subtype of Macrophage Applicants found was termed “Inflammatory Macrophages”, which uniquely expressed inflammatory cytokines such as CCL3, CCL3L1, IL1B, CXCL2, and CXCL3. The remaining “ITGAX high” Macrophages were distinguished from other immune cell types by ITGAX, VCAN, PSAP, FTL, FTH1 and CD163 (though these genes are shared by other specialized macrophages subsets). T cells were largely CD69 and CD8A high, consistent with a T resident memory-like phenotype, and Applicants were not able to resolve a separate cluster of CD4 T cells. Two specialized subtypes of CD8 T Cells were annotated from this dataset: one defined by exceptionally high expression of Early Response genes (FOSB, NR4A2, and CCL5), and the other termed Interferon Responsive Cytotoxic CD8 T Cells, defined by granzyme and perforin expression (GZMB, GZMA, GNLY, PRF1, GZMH), anti-viral genes (ISG20, IFIT3, APOBEC3C, GBP5) and genes associated with effector CD8 T cell function (LAG3, IL2RB, IKZF3, TBX21).

Among immune cells, Macrophages were markedly increased relative to other immune cell types during severe COVID-19 (FIG. 9G, 9H). Multiple specialized myeloid cell types were uniquely detected and enriched among COVID-19 participants, albeit in a subset of participants, and biased to severe COVID-19 cases: ITGAX high Macrophages, FFAR high Macrophages, Inflammatory Macrophages, and Interferon Responsive Macrophages (FIG. 9H). Through rare, plasmacytoid DCs and mast cells were only recovered as >1% of immune cells among COVID-19 participants. Somewhat surprisingly, T Cells and T Cell subtypes were not dramatically altered between disease cohorts. Finally, Applicants assessed the correlation between distinct cell types across all participants. When samples from all disease cohorts were considered, Applicants found that proportional abundance of Dendritic Cells, Mast Cells, and Macrophages were highly correlated with one another (p<0.01), likely indicative of the coordinated recruitment of these immune subtypes during inflammation. Among detailed immune cell types, Interferon Responsive Macrophages were highly correlated with Interferon Responsive Cytotoxic CD8 T Cells (p<0.01), suggesting direct communication between IFNG-expressing tissue resident T cells and CXCL9/10/11 expressing myeloid cells.

These analyses demonstrate how the epithelial and immune compartments are dramatically altered during COVID-19, likely reflecting both protective anti-viral and regenerative responses, as well as pathologic changes underlying progression to severe disease.

### Example 4—Cellular Behaviors Associated with COVID-19 Disease Trajectory

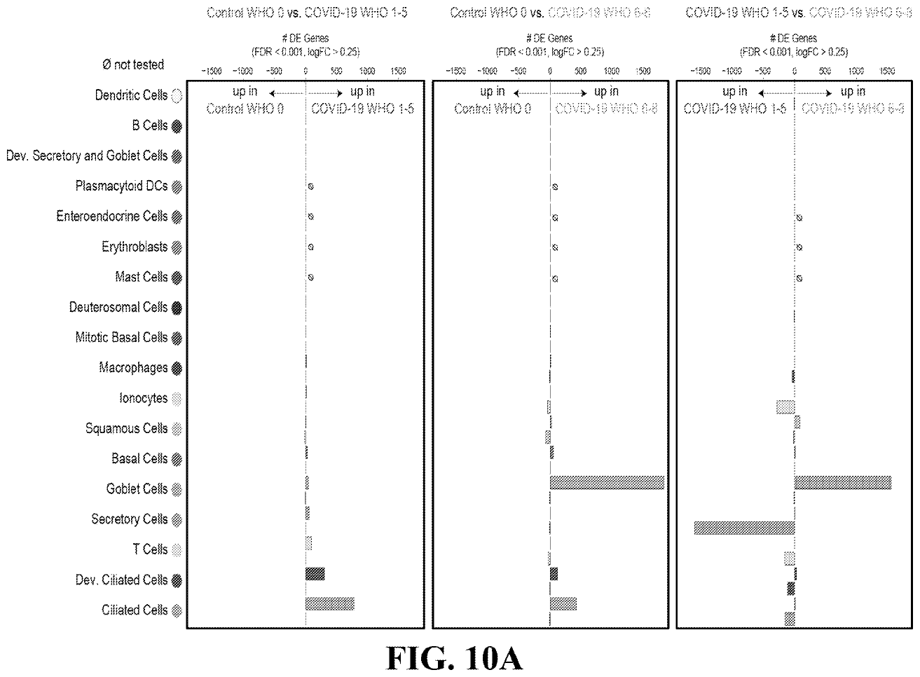

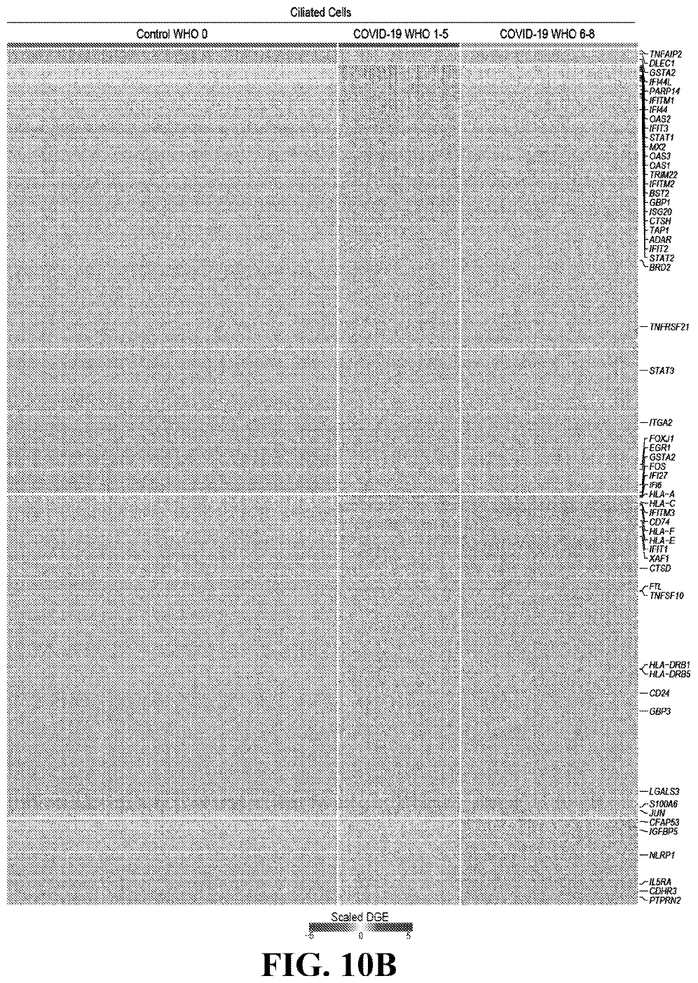

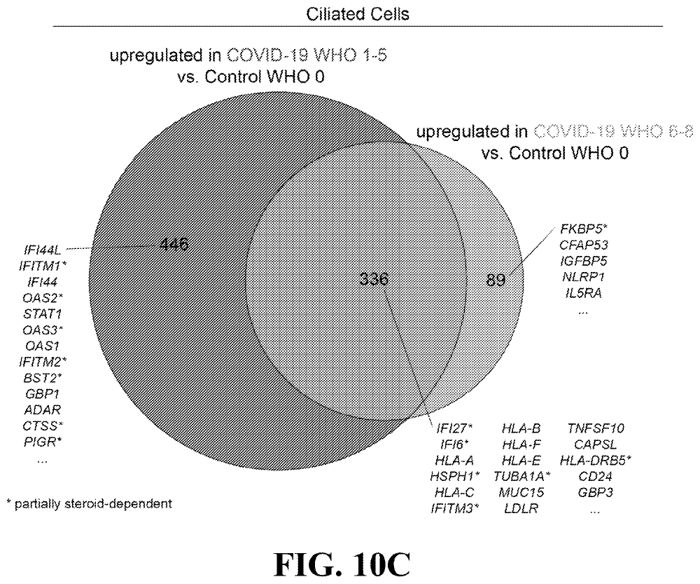

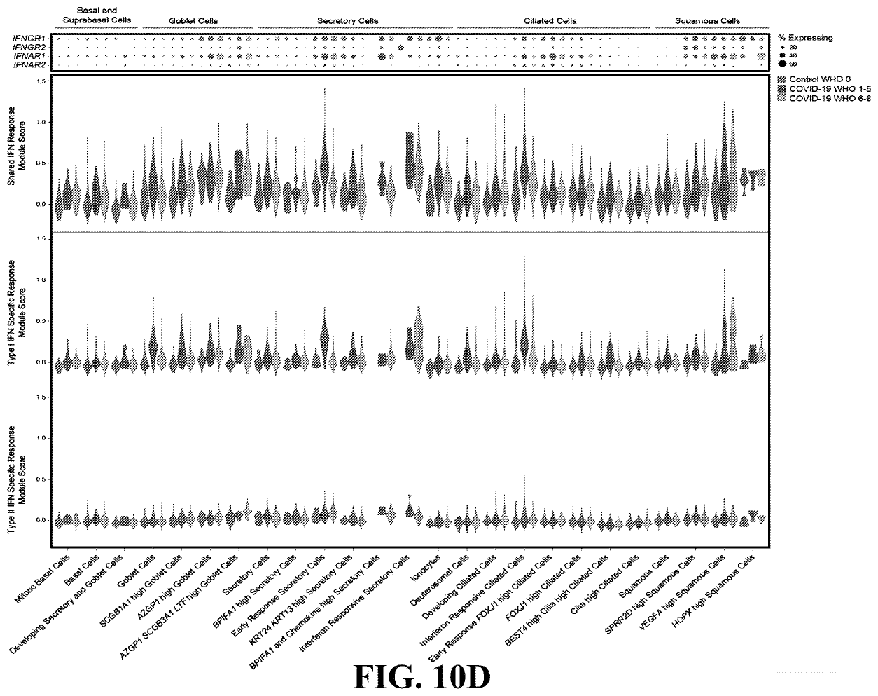

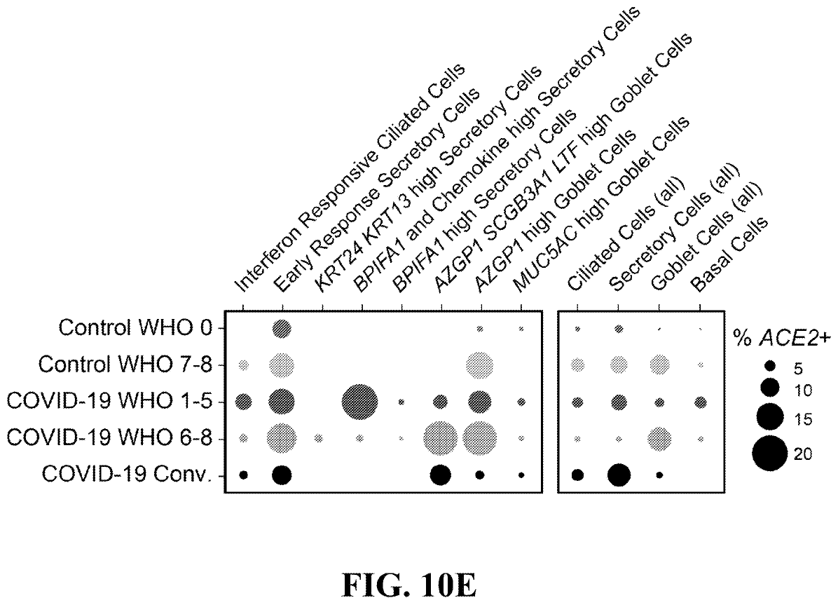

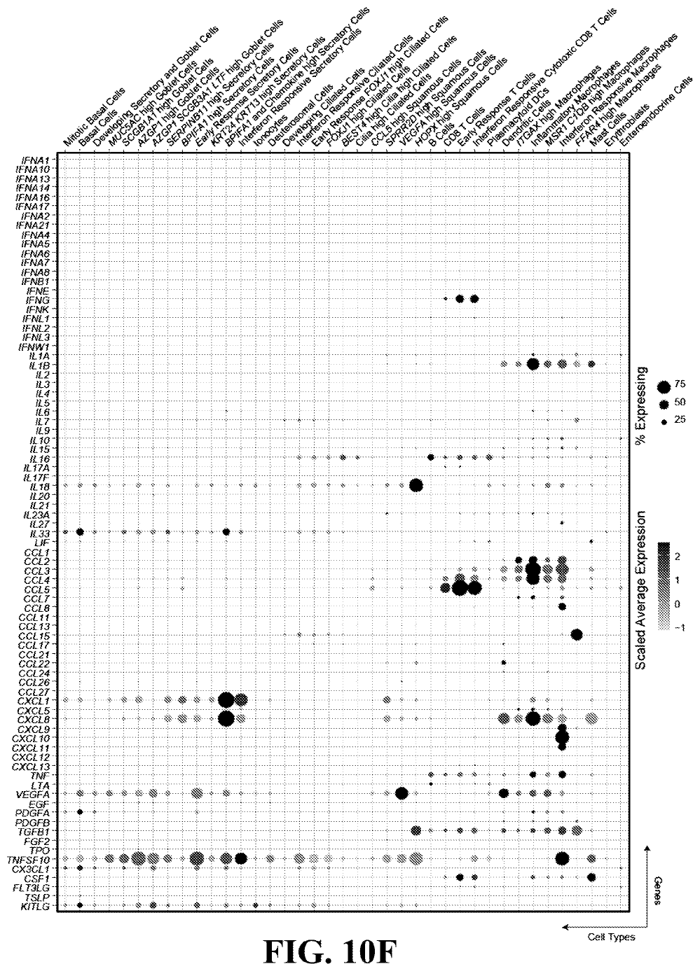

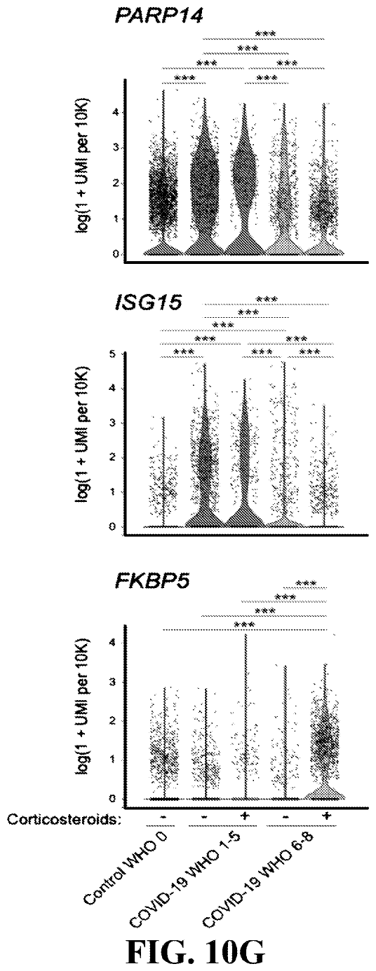

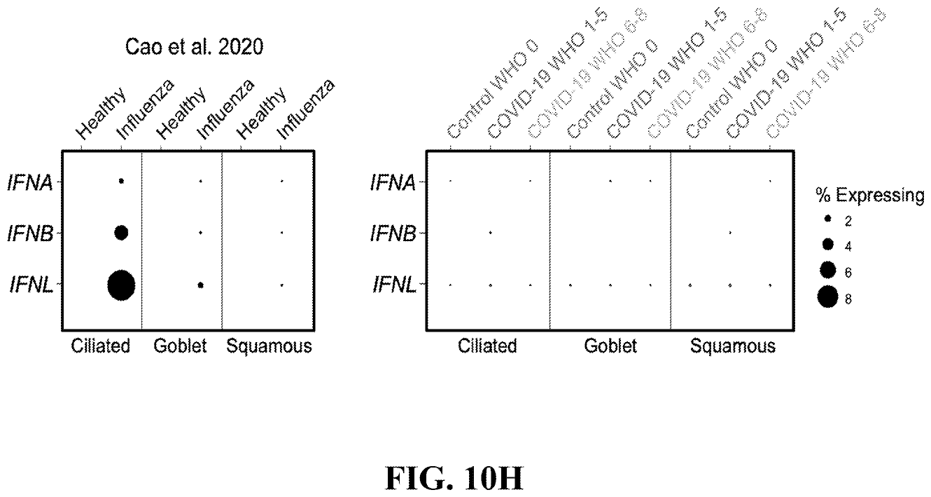

Thus far, Applicants have characterized how COVID-19 elicits major cell compositional changes within the nasopharyngeal mucosa, including expansion of the secretory cell/deuterosomal cell compartments to repopulate lost mature ciliated cells, and recruitment of highly inflammatory myeloid cells. Next, Applicants examined how each individual cell type responds during COVID-19. Here, Applicants restricted the analysis to pairwise comparisons between Control WHO 0, COVID-19 WHO 1-5 (mild/moderate), and COVID-19 WHO 6-8 (severe), and compared both high-level “Coarse” cell types (FIG. 1B, Tables 2-4), and “Detailed” cell subsets (FIGS. 2A, 1D, FIG. 9B). Among all coarse cell types, the largest magnitude transcriptional changes (measured by the number of differentially expressed (DE) genes with FDR <0.001, and log fold change >0.25) were observed primarily within the epithelial compartment, most strikingly within Ciliated Cells, Developing Ciliated Cells, Secretory Cells, Goblet Cells, and Ionocytes (FIG. 10A). Notably, as Applicants had previously discovered substantial heterogeneity among some of these coarse cell types, namely Secretory and Goblet Cells, it is unsurprising that many of these differentially expressed genes (e.g., between Goblet Cells from Control WHO 0 participants vs. Goblet Cells from COVID-19 WHO 6-8 participants) reflect novel cellsubtypes that emerge or dominate during COVID-19 and may partially confound true “cell-type intrinsic” transcriptional responses. Therefore, Applicants similarly compared transcriptomic responses among the detailed cell type annotations between disease cohorts (FIG. 3A). Here, the largest transcriptional changes were found among AZGP1 high Goblet Cells, Early Response FOXJ1 high Ciliated Cells, FOXJ1 high Ciliated Cells, Goblet Cells, SERPINB11 high Secretory Cells, Early Response Secretory Cells, and Interferon Responsive Ciliated Cells. Broadly, major differences were observed in the identity of cell types with large transcriptional responses—with mild/moderate COVID-19 driving differences principally in multiple Ciliated Cell subtypes, MUC5AC high Goblet Cells, and Ionocytes, while severe COVID-19 included major perturbations among Basal cells, AZGP1 high Goblet Cells, and various Ciliated Cell types. Finally, when Applicants directly compared mild/moderate to severe COVID-19, multiple cell types showed robust differential gene expression, most drastically among Ciliated Cell subtypes (Interferon Responsive Ciliated Cells, FOXJ1 high Ciliated Cells, Early Response FOXJ1 high Ciliated Cells, Developing Ciliated Cells), Ionocytes, SERPINB11 high Secretory Cells, Early Response Secretory Cells, and AZGP1 high Goblet Cells.

First, Applicants examined the specific DE genes among Ciliated Cells (all, coarse annotation) between each cohort (FIG. 3B, Tables 2-4). Compared to Ciliated Cells from Control WHO 0 participants, cells from both mild/moderate COVID-19 and severe COVID-19 robustly upregulated genes involved in the host response to virus, including IFI27, IFIT1, IFI6, IFITM3, and GBP3, and both cohorts induced expression of MHC-I and MHC-II genes (including HLA-A, HLA-C, HLA-F, HLA-E, HLA-DRB1, HLA-DRA) and other factors involved in antigen processing and presentation (FIGS. 10B, 10C). Notably, large sets of interferon-responsive and anti-viral genes were exclusively induced among Ciliated Cells from COVID-19 WHO 1-5 participants when compared to Control WHO 0 participants, and in a direct comparison of Ciliated Cells from mild/moderate COVID-19 to severe COVID-19, the cells from individuals with mild/moderate disease showed strong upregulation of diverse anti-viral factors, including IFI44L, STAT1, IFITM1, MXJ, IFITM3, OAS1, OAS2, OAS3, STAT2, TAP1, HLA-C, ADAR, XAF1, IRF1, CTSS, CTSB, and many others. Ciliated Cells from severe COVID-19 uniquely upregulated IL5RA and NLRP1 (compared to both control and mild/moderate COVID-19). Together, these differentially expressed gene lists are suggestive of exposure to secreted inflammatory factors and type I/II/III interferons, as well as direct cellular sensing of viral products. Using previously published data from human nasal basal cells treated in vitro with either type I (IFNA) or type II (IFNG) interferon³⁶, Applicants created gene sets that represented the “shared” gene responses to type I and type II interferon, and the cellular responses specific to either type (FIG. 3B). Using gene set enrichment analysis, Applicants tested whether the genes that discriminate Ciliated cells from different disease cohorts (e.g., mild/moderate COVID-19 vs. severe COVID-19) imply exposure to specific interferon types. Applicants found that Ciliated cells in mild/moderate COVID-19 robustly induced type I interferon-specific gene signatures, both compared to cells from healthy controls, as well as individuals with severe COVID-19. Conversely, only a few genes were suggestive of a type II response, including induction of NMC-II genes among mild/moderate COVID-19 cases. Further, when compared to healthy individuals, Ciliated cells from individuals with severe COVID-19 did not significantly induce type I or type II interferon responsive genes, potentially underlying poor control of viral spread.

Applicants next investigated whether these effects were observed among other cell types and subsets. Surprisingly, even among cells defined as “Interferon Responsive” Ciliated Cells, cells from mild/moderate COVID-19 participants expressed higher fold changes of interferon-responsive genes compared to cells from COVID-19 WHO 6-8 participants or Control WHO 0 (FIGS. 3C, 3D, Tables 2-4). Other detailed epithelial cell types displayed a similar pattern: where broad interferon-responsive genes (largely type I specific) were strongly upregulated among cells from mild/moderate COVID-19 participants, while cells from severe COVID-19 upregulated few shared markers with mild/moderate COVID-19 participants, and instead skewed towards inflammatory genes such as S100A8 and S100A9 instead of anti-viral factors (FIG. 3E-3H). In some cases, cells from individuals with severe COVID-19 expressed levels of interferon responsive or anti-viral genes indistinguishable from healthy controls. Strongest induction of type I specific interferon responses among mild/moderate COVID-19 cases was observed in MUC5AC high Goblet Cells, SCGB1A1 high Goblet Cells, Early Response Secretory Cells, Deuterosomal Cells, Interferon Responsive Ciliated Cells, and BEST4 high Cilia high Ciliated Cells (FIG. 3G). Rare cell types from severe COVID-19 individuals induced comparable type I interferon responses to their mild/moderate counterparts, including AZGP1 SCGB3A1 LTF high Goblet Cells, Interferon Responsive Secretory Cells, and VEGFA high Squamous Cells. Expression of type II specific genes were globally blunted across all cell types from COVID-19 samples when compared to type I module scores (FIG. 3G, FIG. 10D). Further, the absence of a transcriptional response to secreted interferon could not be explained by a lack of either interferon alpha receptor (IFNAR1, IFNAR2) or interferon gamma receptor (IFNGR1, IFNGR2) expression. Previous work has identified ACE2, the host receptor for SARS-CoV-2, as among the interferon-induced genes in nasal epithelial cells. Indeed, Applicants observe modest upregulation of this gene among cells from COVID-19 participants compared to healthy controls. Further, some of the cell subtypes identified as expanded during COVID-19 (e.g., Interferon Responsive Ciliated Cells, BPIFA1 high Secretory Cells, BPIFA1 and Chemokine high Secretory Cells, and KRT24 KRT13 high Secretory Cells) express relatively high abundances of ACE2 (FIG. 10E).

Together, across all detailed cell types, cells within the COVID-19 WHO 1-5 cohort recurrently upregulated interferon-responsive factors including STAT1, MXJ, HLA-B, HLA-C, among others (compared to matched cell types among Control WHO 0 participants), while cells from the COVID-19 WHO 6-8 cohort repeatedly induced a distinct set of genes, including S100A9, S100A8 and stress response factors (HSPA8, HSPA1A, DUSP1, FIG. 3H).

Applicants were curious as to whether depressed interferon and anti-viral responses could be explained by higher rates of steroid treatment among the severe COVID-19 group (Table 1). Applicants therefore stratified the cohorts further into Steroid-Treated vs. Untreated, and assessed expression of genes previously identified as DE between Control WHO 0, COVID-19 WHO 1-5, and COVID-19 WHO 6-8. For some genes, steroid treatment partially suppressed the interferon response within each cohort—for instance, Ciliated Cells from Untreated COVID-19 WHO 1-5 participants showed higher abundances of IFITM1, OAS2, IFI6, and IFI27 than their Steroid-Treated counterparts—while still maintaining strong differences in expression between cohorts (with abundance in COVID-19 WHO 1-5>COVID-19 WHO 6-8>COVID-19 WHO 0, see annotations on FIG. 10C). Intriguingly, induction of FKBP5 expression among Ciliated Cells from severe COVID-19 participants was fully explained by steroid treatment, which is consistent with the role for this protein in modulating glucocorticoid receptor activity. Other sets of anti-viral genes were equivalently expressed within each cohort, independent of steroid treatment, including STAT1, STAT2, IFI44, and ISG15. For many anti-viral factors in multiple cell types, Applicants observed no effect of steroid treatment on the intrinsic anti-viral response during COVID-19.

Together, these data demonstrate global blunting of the local anti-viral/interferon response among nasopharyngeal epithelial cells during severe COVID-19. Applicants next attempted to query the source of local interferon, particularly in the COVID-19 WHO 1-5 samples where cell types appeared to be maximally responding to interferon stimulation. Notably, Applicants expect many of the tissue-resident immune cells to reside principally within the deeper lamina propria and submucosal spaces, and are therefore are poorly represented in the dataset due to sampling type (swabbing of surface epithelial cells). Accordingly, Applicants found exceedingly few immune cell types producing interferons: IFNA and IFNB were absent, rare IFNL1 UMI were observed among T cells and Macrophages, and IFNG was robustly produced from cytotoxic CD8 T cells, despite limited evidence for type II responses among epithelial cells (FIG. 10F). Further, Applicants could not detect expression of any interferon types among epithelial cells, which is dramatically different from previous observations of robust type I/III interferon expression among nasal ciliated cells during influenza A and B infection (FIG. 10G). Rather, Applicants found robust induction of other inflammatory molecules from both immune and epithelial cell types. CXCL8 was produced by several specialized secretory cell types, including those uniquely expanded in COVID-19. Inflammatory and Interferon Responsive Macrophages represent the primary sources of local TNF, IL6, and IL10, and uniquely express high abundances of chemoattractant molecules such as CCL3, CCL2, CXCL8, CXCL9, CXCL10, and CXCL11 (FIG. 10F).

Applicants directly tested whether the lack of an IFN-stimulated response among nasal epithelial cells in severe COVID-19 participants could be explained by autoantibody mediated inhibition of secreted interferons as reported in other cohorts (Bastard, P., et al. (2020). Autoantibodies against type I IFNs in patients with life-threatening COVID-19. Science (80); Bastard, P., et al. (2021). Preexisting autoantibodies to type I IFNs underlie critical COVID-19 pneumonia in patients with APS-1. J. Exp. Med. 218; Wang, N., et al. (2020a). Retrospective Multicenter Cohort Study Shows Early Interferon Therapy Is Associated with Favorable Clinical Responses in COVID-19 Patients. Cell Host Microbe). Using matched plasma collected at the time of NP swab, Applicants analyzed a subset of 25 participants for IgG and RgM antibodies targeting a large panel of potential antigens (using a microarray-based antibody hybridization platform, see Methods). Here Applicants found evidence for IgG autoantibodies targeting IFN-ω and 11 IFNα, subtypes in 1/8 participants who developed severe COVID-19, 0/12 participants with mild/moderate disease, and 0/5 healthy donors (FIG. 3I). Applicants caution against generalizing this result due to the limited cohort size; Applicants note however that the findings agree well with the expected proportion (˜10%) of severe individuals with autoantibodies to IFN-components from published data (Bastard et al., 2020).

To better understand participant-to-participant variability in anti-viral and IFN-responsive gene signatures, Applicants analyzed the average expression of STAT1, STAT2, IRF1, and IRF9—key transcription factors responsible for the induction of IFN-stimulated gene expression and IFN-induced genes themselves—among ciliated cells from each participant (FIG. 3J). Applicants found that the expression of STAT1, STAT2, and IRF1 was indistinguishable among cells from control WHO 0, control WHO 7-8, and COVID-19 WHO 6-8 participants. IRF9 was diminished among COVID-19 WHO 6-8 participants and control WHO 7-8 participants compared to healthy donors and participants with mild or moderate COVID-19. Intriguingly, despite the absence of autoantibodies directed at type I interferons, nearly all participants who developed severe COVID-19 failed to induce STAT1, STAT2, IRF1, and IRF9 expression (among other IFN-stimulated genes). Even individuals who had milder disease and limited requirement for respiratory support at the time of nasal swab, but later went on to develop severe or fatal COVID-19 (swab WHO 1-5, peak WHO 6-8), already had diminished STAT1 expression at the time of nasal swab (FIG. 3J). This suggests a potential predictive value of poor interferon-stimulated gene (ISG) induction.

### Example 5—Targets of SARS-CoV-2 Infection in the Nasopharynx

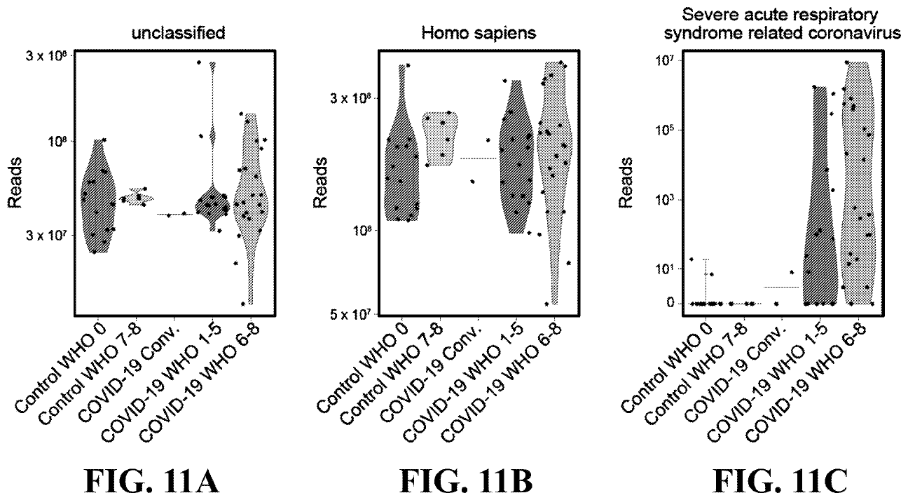

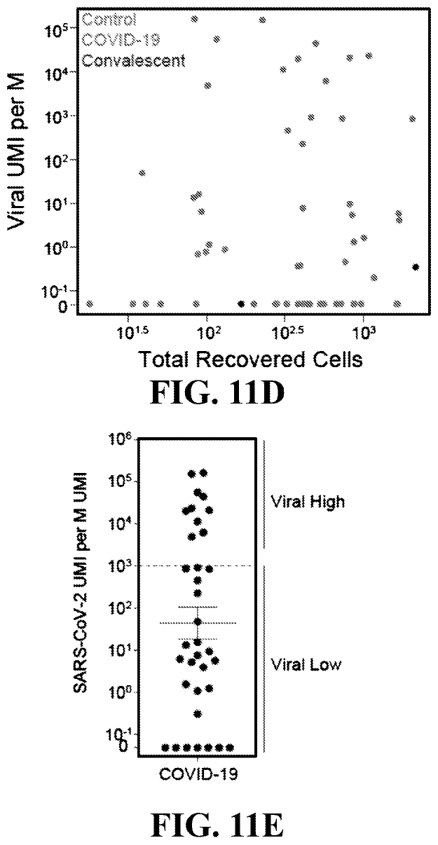

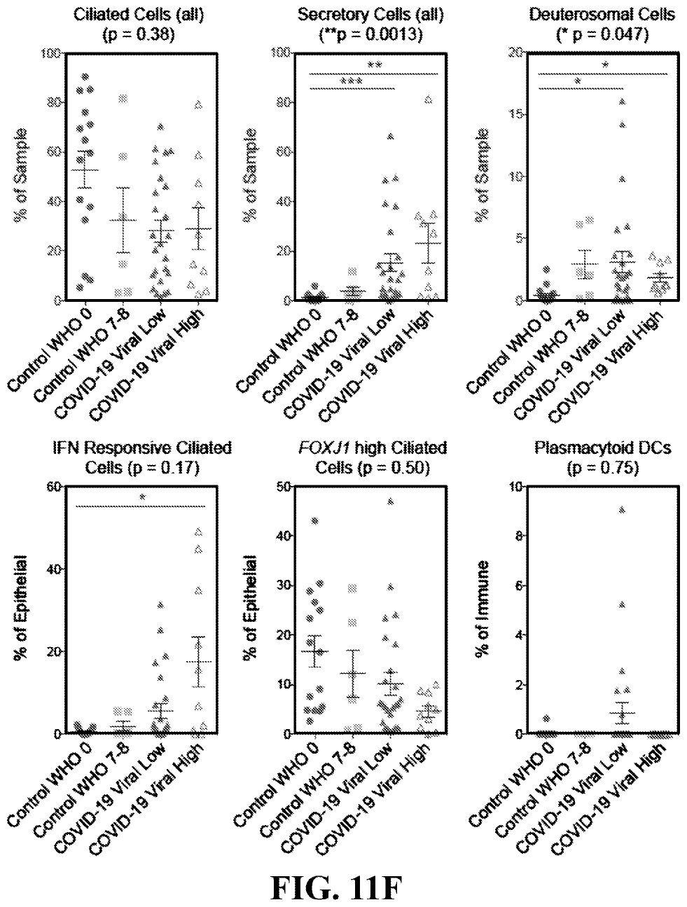

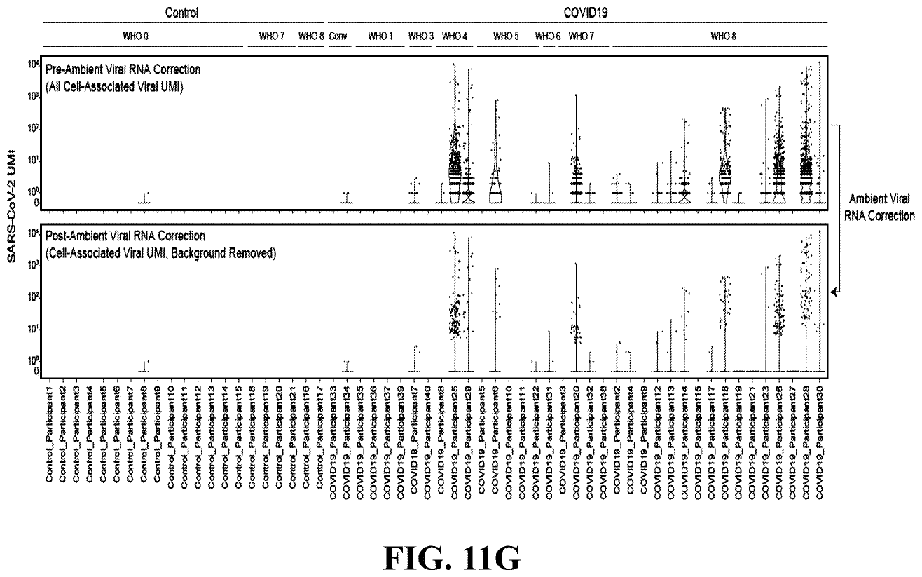

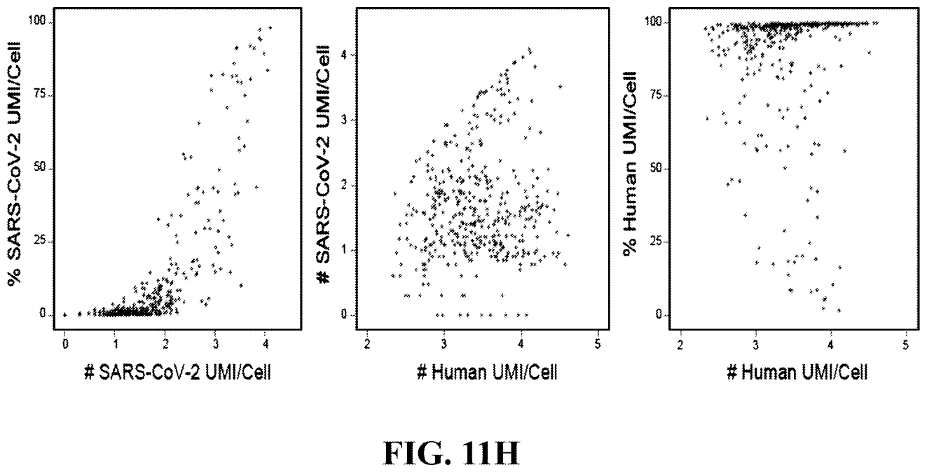

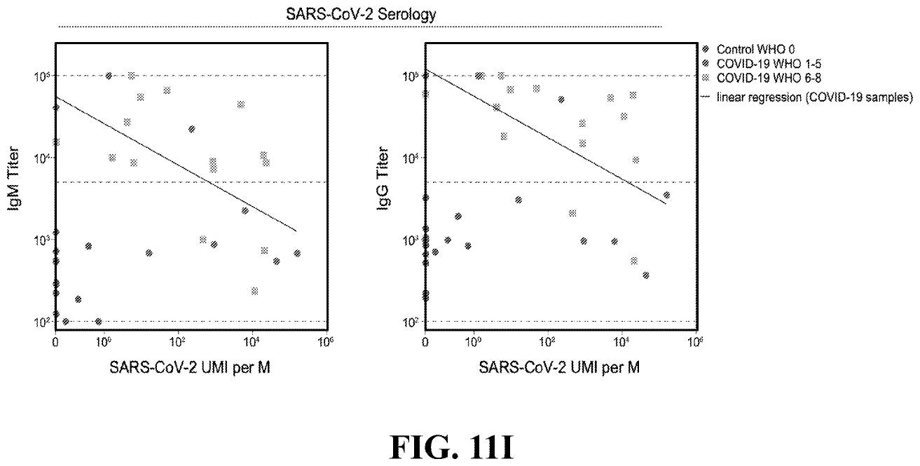

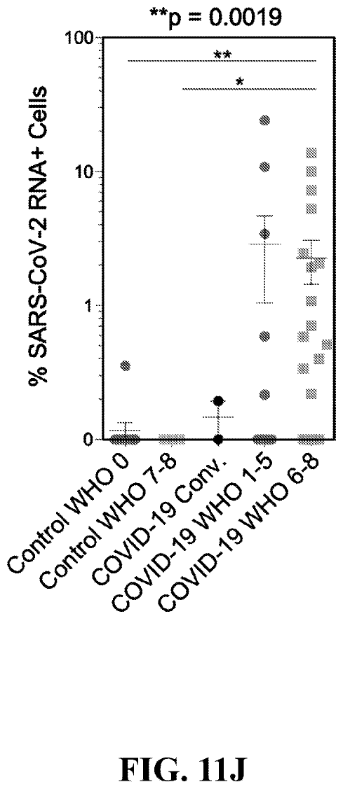

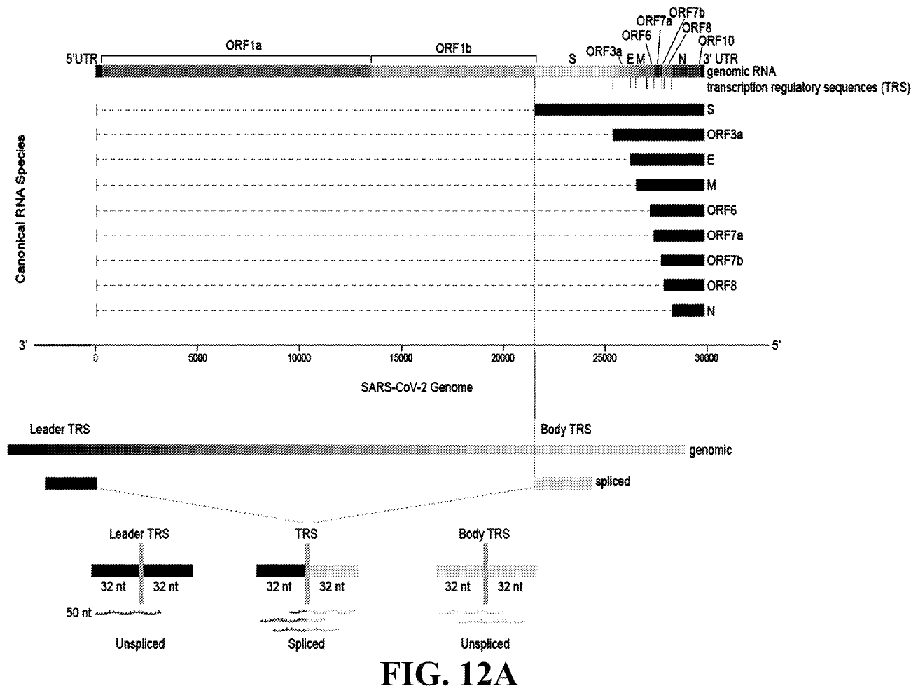

Given a comprehensive picture of host cell biology during COVID-19 and across the spectrum of disease severity, Applicants next tested whether the observed epithelial phenotypes were associated with altered viral loads. Single cell RNA-sequencing protocols utilize poly-adenylated RNA capture and reverse transcription to generate snapshots of the transcriptional status of each individual cell. As other pathogens and commensal microbes also utilize poly-adenylation for RNA intermediates, or contain poly-adenylated stretches of RNA within their genomes, they may also be represented within single-cell RNA-seq libraries. First, to perform an unbiased search for co-detected viral, bacterial, and fungal genomic material, Applicants used metatranscriptomic classification (implemented with Kraken2) to assign reads according to a comprehensive reference database (previously described, see Methods). As expected, the majority (28/38) of swabs from individuals with COVID-19 contained reads classified as SARS coronavirus species (FIG. 4A, FIGS. 11A-11C). Among samples containing SARS coronavirus genomic material, the read abundance ranged from 2e0 to 8.8e6 reads (1.8e-3 to 1.9e4 reads/M total reads). Applicants found little evidence for co-occurring respiratory viral infections, which may be partially explained by the season when many of the swabs were collected (April-September 2020) and concurrent social distancing practices. Swabs from two individuals were found to contain rare reads classified as Influenza A virus species (maximum 5 reads per donor, within range for spurious classification), and Applicants found no evidence for other seasonal human coronaviruses, Influenza B virus, metapneumovirus, or orthopneumovirus. Swabs from two individuals with mild/moderate COVID-19 were found to contain exceptionally high abundances of reads classified as Rhinovirus A (2.1e5 and 2.4e5 reads). Finally, Applicants recovered SARS coronavirus assigned reads from two participants from the Control WHO 0 cohort and one individual classified as Convalescent (>40 days following resolution of mild COVID-19).

Next, Applicants analyzed all SARS-CoV-2-aligned UMI following alignment to a joint genome containing both human and SARS-CoV-2. Applicants took the sum of all SARS-CoV-2 aligning UMI from a given participant—both from high-quality single-cell transcriptomes and low-quality/ambient RNA—as a representative measure of the total SARS-CoV-2 burden within the tissue microenvironment. As observed using metatranscriptomic classification, Applicants found relatively low/spurious alignments to SARS-CoV-2 among Control participants, while swabs from COVID-19 participants contained a wide range of SARS-CoV-2 aligning reads (FIG. 4B, FIGS. 11D, 11E). Samples from COVID-19 WHO 6-8 participants contained significantly higher abundances of SARS-CoV-2 aligning reads than both control cohorts, with an average of 1.1e2+/−2.8e0 (geometric mean+/−SEM) UMI per million aligned UMI (ranging from 0 to 1.5e5 per sample). Swabs from participants with mild/moderate COVID-19 contained slightly fewer SARS-CoV-2 aligning UMI, with an average of 1.1e1+/−4.3e0 (geometric mean+/−SEM) UMI per M.

Given the large diversity in SARS-CoV-2 abundance across all COVID-19 participants, Applicants interrogated whether cell composition correlated with total SARS-CoV-2 (NB: contemporaneous work by Applicants has evaluated the accuracy of single-cell RNA-seq derived estimates of total SARS-CoV-2 abundance with more established protocols such as Real-Time RT-PCR). Among all cell types, Applicants found that Secretory Cells were significantly positively correlated with the total viral abundance (Spearman's rho=0.49, Bonferroni-corrected p=0.0015), while FOXJ1 high Ciliated Cells were significantly negatively correlated (Spearman's rho=−0.43, Bonferroni-corrected p=0.020, FIG. 4C, 4D). This observation is in line with findings outlined in FIGS. 1 and 2 where epithelial cell destruction during SARS-CoV-2 infection drives preferential loss of differentiated ciliated cell types, and secretory cells may expand to repopulate lost epithelial cell types. Next, Applicants binned the samples from COVID-19 participants into “Viral Low” and “Viral High” groupings (based on an arbitrary cutoff of 1e3 SARS-CoV-2 UMI per M, though the findings were robust to a range of partition choices, FIGS. 11E, 11F). Interferon Responsive Ciliated Cells were expanded among “Viral High” COVID-19 samples and plasmacytoid DCs were absent from “Viral High” samples.

Next, Applicants aimed to differentiate SARS-CoV-2 UMI derived from ambient or low-quality cell barcodes from those truly reflecting intracellular RNA molecules. First, Applicants filtered to only viral UMIs associated with cells presented in FIG. 1, thereby removing those associated with low-quality cell barcodes (FIG. 11G). Next, using a combination of computational tools to 1) estimate the proportion of ambient RNA contamination per single cell and 2) estimate the abundance of SARS-CoV-2 RNA within the extracellular/ambient environment (i.e., not cell-associated), Applicants were able to test whether the amount of viral RNA associated with a given single-cell transcriptome was significantly higher than would be expected from ambient spillover. Together, this enabled Applicants to identify cell barcodes whose SARS-CoV-2 aligning UMI were likely driven by spurious contamination, and annotate single cells that contain probable cell-associated or intracellular SARS-CoV-2 RNA (FIG. 4E, FIG. 11G). Across all single cells, this analysis recovered 415 high-confidence SARS-CoV-2 RNA+ cells across 21 participants, and Applicants confirmed that cell assignment as “SARS-CoV-2 RNA+” was not driven by technical factors such as sequencing depth or cell complexity (FIG. 11H). 262 cells (of 12,909) were from participants with severe COVID-19 and 150 (of 5,194) from mild/moderate COVID-19. Applicants found 3 SARS-CoV-2 RNA+ cells from participants with negative SARS-CoV-2 PCR: two from a participant classified as “Convalescent”, and one from a Control participant. Among participants with any SARS-CoV-2 RNA+ cell, Applicants found 20+/−7 (mean+/−SEM) SARS-CoV-2 RNA+ cells per sample (range 1-119), amounting to 4+/−1.3% (range 0.1-24%) of the recovered cells per sample. Within a given single cell, the abundance of SARS-CoV-2 UMI ranged from 1 to 12,612, corresponding to 0.01-98% of all human and viral UMI per cell.

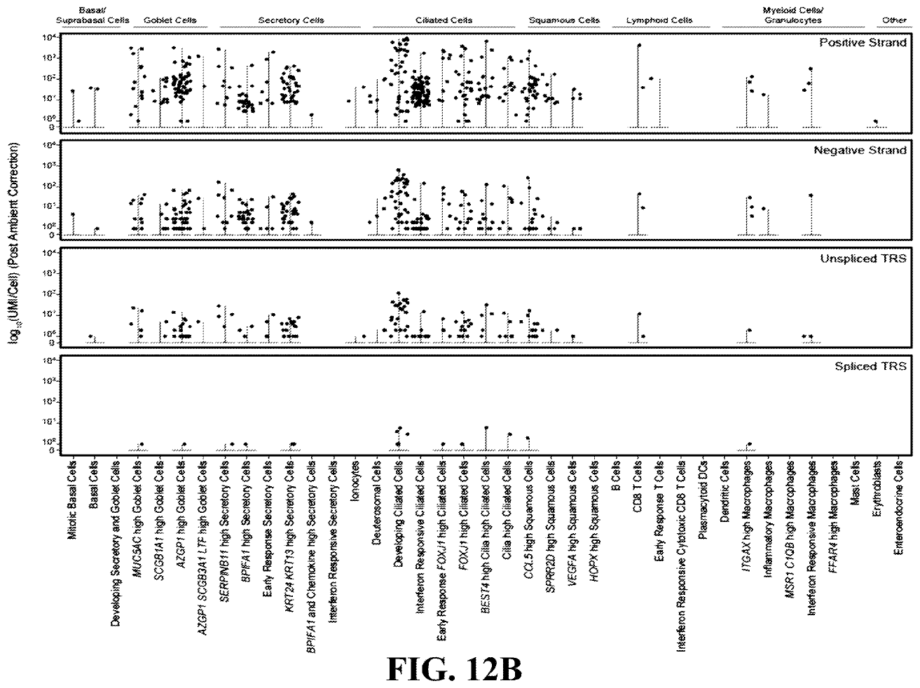

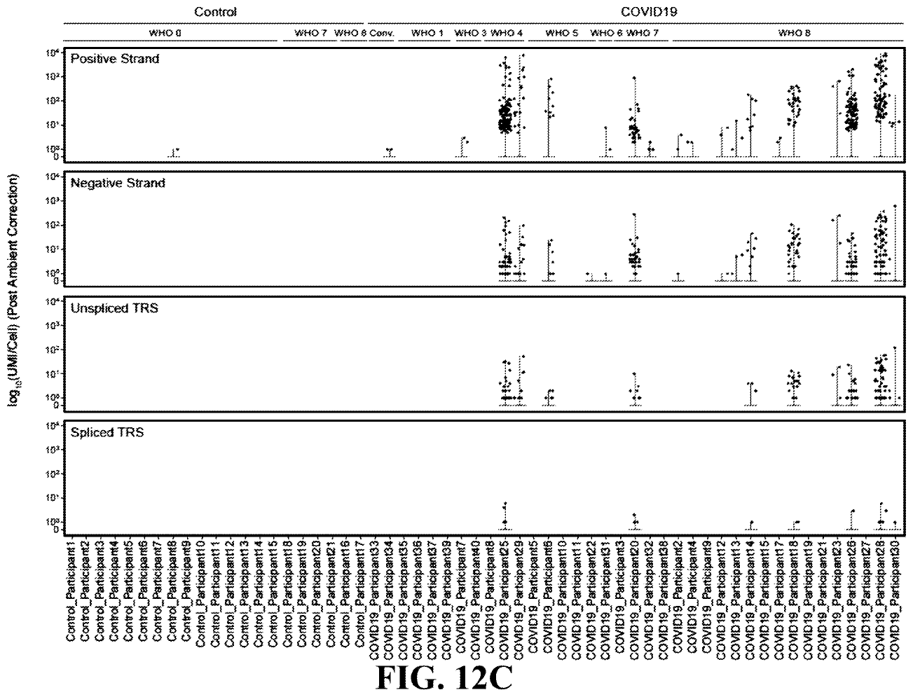

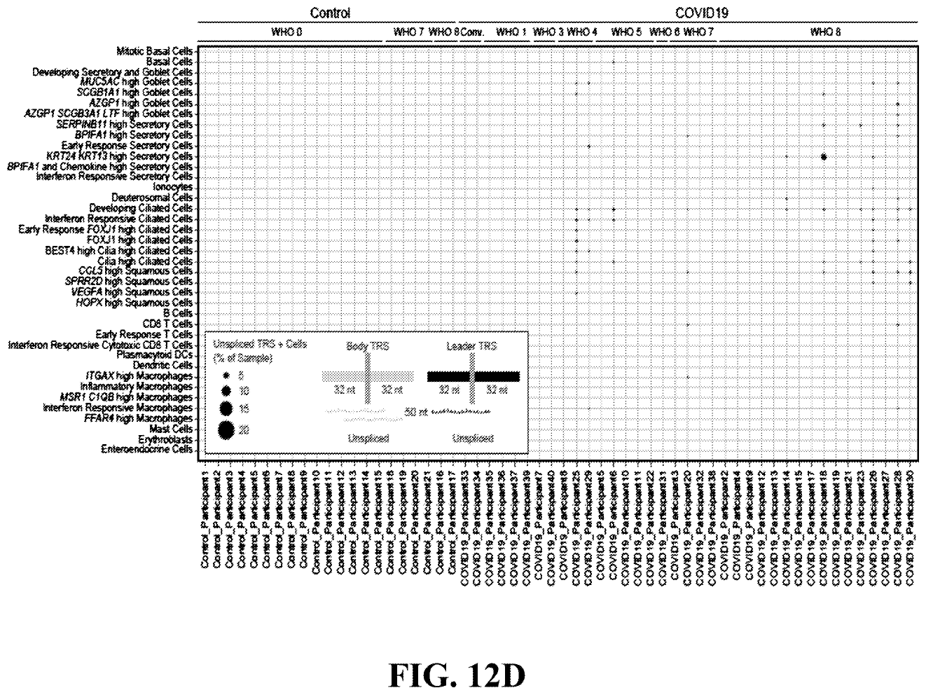

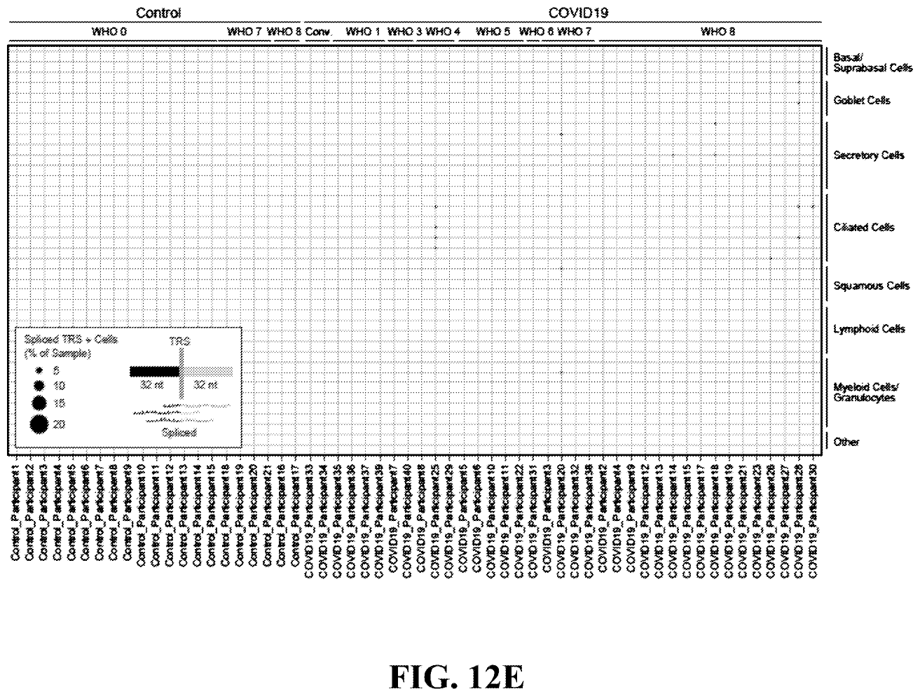

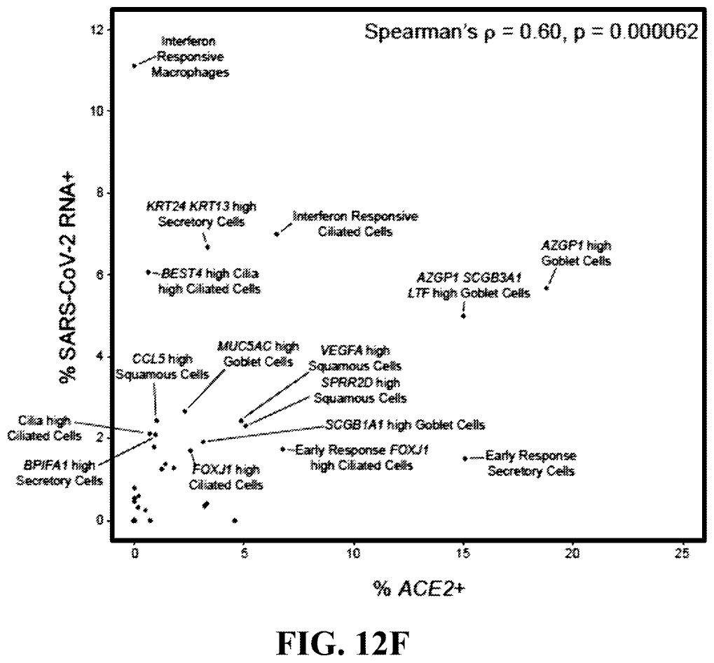

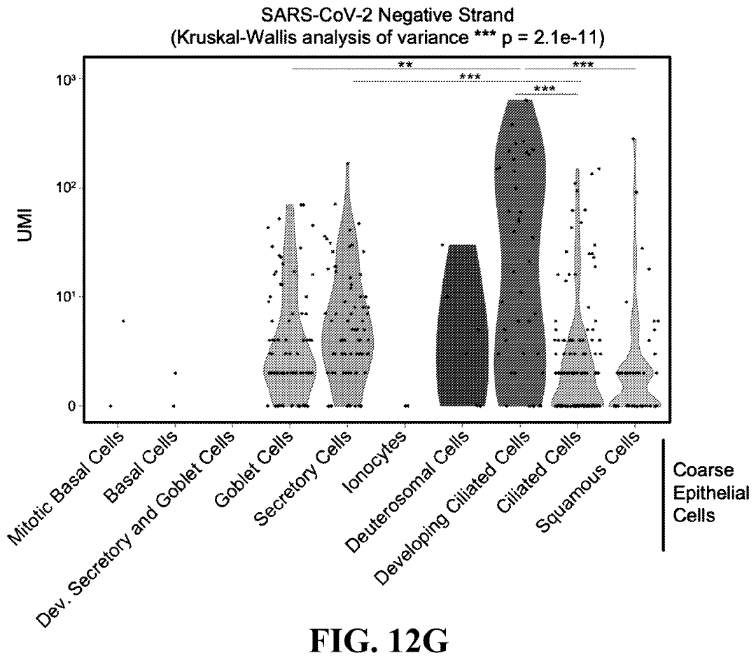

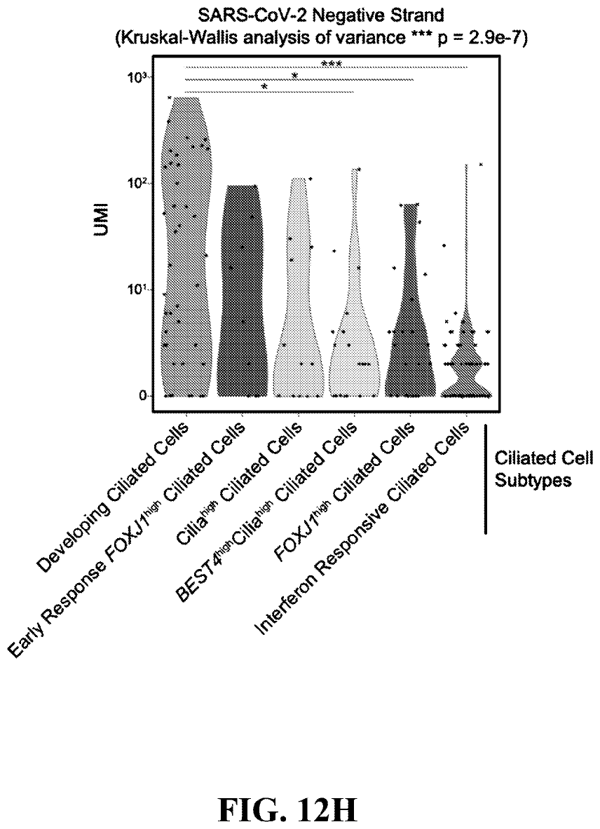

To further understand the biological significance behind SARS-CoV-2 aligning UMI within a single cell, and to better identify cells with the highest-likelihood of actively supporting viral replication, Applicants analyzed the specific viral sequences and their alignment regions in the viral genome. During SARS-CoV-2 infection, viral uncoating from endosomal vesicles releases the positive, single-stranded, 5′ capped, poly-adenylated genome into the host cytosol (FIG. 4F, 4G). Here, translation of non-structural proteins proceeds first by templating directly off of the viral genome, generating a replication and transcription complex. The viral replication complex then produces both 1) negative strand genomic RNA intermediates, which serve as templates for further positive strand genomic RNA and 2) nested subgenomic mRNAs which are constructed from a 5′ leader sequence fused to a 3′ sequence encoding structural proteins for production of viral progeny (e.g., Spike, Envelope, Membrane, Nucleocapsid). Generation of nested subgenomic mRNAs relies on discontinuous transcription occurring between pairs of 6-mer transcriptional regulatory sequences (TRS), one 3′ to the leader sequence (termed leader TRS, or TRS-L), and others 5′ to each gene coding sequence (termed body TRS, or TRS-B). Applicants reasoned that short SARS-CoV-2 aligning UMI could be readily distinguished by their strandedness (aligning to the negative vs. positive strand) and whether they fell within coding regions, across intact TRS (indicating RNA splicing had not occurred for that RNA molecule at that splice site) or across a TRS with leader-to-body fusions (corresponding to subgenomic RNA, FIG. 4F, 4G, FIG. 12A). Single cells containing higher abundances of spliced or negative strand aligning reads are therefore more likely to represent truly virally infected cells with a functional viral replication and transcription complex. Critically, the co-detection of host transcriptomic and viral genomic material associated with a single cell barcode cannot definitively establish the presence of intracellular virus and/or productive infection. Rather, Applicants integrate these and other aspects of the host and viral transcriptomes to refine and contextualize the confidence in “SARS-CoV-2 RNA+” cells.

The majority of SARS-CoV-2 aligning UMI among SARS-CoV-2 RNA+ cells was found heavily biased towards the 3′ end of the genome, attributed to the 3′ UTR, ORF10, and N gene regions, as expected due to poly-A priming (FIG. 4H). A majority (68.7%) of SARS-CoV-2 RNA+ cells contained reads aligning to the viral negative strand, increasing the likelihood that many of these cells represent true targets of SARS-CoV-2 virions in vivo. In addition to negative strand alignment, Applicants find roughly ˜¼ of the SARS-CoV-2 RNA+ cells contain at least 100 UMI that map to more than 20 distinct viral genomic locations per cell. Finally, comparing spliced to unspliced UMI, Applicants found a minor fraction of cells with reads mapping directly across a spliced TRS sequence (4.6%), while 35% of SARS-CoV-2 RNA+ cells contained reads mapping across the equivalent 70mer window around an unspliced TRS. Notably, single cells containing reads aligning to spliced (subgenomic) RNA were heavily skewed toward those cells that contained the highest overall abundances of viral UMI—this may be an accurate reflection of coronavirus biology, wherein subgenomic RNA are most frequent within cells robustly producing new virions and total viral genomic material, but also points to inherent limitations in the detection of low-frequency RNA species by single-cell RNA-seq technologies.

Next, Applicants integrated 1) the strand and splice information among SARS-CoV-2 aligning UMIs, 2) participant-to-participant diversity and 3) cell type annotations to gain a comprehensive picture of the identity and range of SARS-CoV-2 RNA+ cells within the nasopharyngeal mucosa (FIG. 5A-D, FIG. 12A-12E). Applicants found incredible diversity in both the identity of SARS-CoV-2 RNA+ cells, as well as the distribution of SARS-CoV-2 RNA+ cells within and across participants. The majority of SARS-CoV-2 RNA+ cells were Ciliated, Goblet, Secretory or Squamous. Highest-confidence SARS-CoV-2 RNA+ cells (spliced UMI, negative strand UMI, >100 SARS-CoV-2 UMI) tended to be found among MUC5AC high Goblet Cells, AZGP1 high Goblet Cells, BPIFA1 high Secretory Cells, KRT24 KRT13 high Secretory Cells, CCL5 high Squamous Cells, Developing Ciliated Cells, and each Ciliated Cell subtype. A high proportion of Interferon Responsive Macrophages contained SARS-CoV-2 genomic material, and rare ITGAX high Macrophages were found to contain UMI aligning to viral negative strand or spliced TRS regions—likely representing myeloid cells that have recently engulfed virally-infected epithelial cells or free virions. Applicants did not find major differences in the presumptive cellular tropism by the severity of COVID-19. A few cell types were commonly found to be SARS-CoV-2 RNA+ across all participants (including participants with only rare viral RNA+ cells): most frequently, participants had at least one Developing Ciliated or Squamous cell with SARS-CoV-2 RNA, followed by Goblet Cells, Cilia high Ciliated Cells, and FOXJ1 high Ciliated Cells (FIG. 5C). However, among the individuals with the highest abundances of SARS-CoV-2 RNA+ cells, viral RNA was spread broadly across many different cell types, including those outside of the expected tropism for SARS-CoV-2 (e.g., also found within Basal Cells, Ionocytes). Further, the cell types harboring the highest proportions of SARS-CoV-2 RNA+ cells represent the same cell types uniquely expanded or induced within COVID-19 participants, such as KRT24KRT13 high Secretory Cells, AZGP1 high Goblet Cells, and Interferon Responsive Ciliated Cells, and contain the highest abundances of ACE2-expressing cells (FIG. 5C, FIG. 12F. Whether these cell types represent specific phenotypes elicited by intrinsic viral infection (potentially alongside induction of anti-viral genes) or are uniquely susceptible to SARS-CoV-2 entry (e.g., enhanced entry factor expression) will require further investigation. Developing ciliated cells contain among the highest SARS-CoV-2 RNA molecules per-cell, including positive strand, negative strand-aligning reads, and spliced TRS reads (FIG. 12G). Among ciliated cell subtypes, IFN responsive ciliated cells, despite representing one of the most frequent “targets” of viral infection, contain the lowest per-cell abundances of SARS-CoV-2 RNA, potentially reflecting the impact of elevated anti-viral factors curbing high levels of intracellular viral replication (FIG. 12H).

### Example 6—Cell Intrinsic Responses to SARS-CoV-2 Infection

Above, Applicants carefully mapped the specific cell types and states harboring SARS-CoV-2 RNA+ cells, identifying the subsets of epithelial cells that appear to actively support viral replication in vivo across distinct individuals (FIG. 5). Further, Applicants have characterized robust and cell-type-specific host responses among cells from COVID-19 participants, ostensibly representing both the bystander cell response to local virus and an inflammatory microenvironment, as well as the intrinsic response to intracellular SARS-CoV-2 RNA (FIG. 3). Here, by directly comparing single cells containing SARS-CoV-2 RNA to their matched bystanders, Applicants aimed to map both the cell-intrinsic response to direct viral infection, as well as the host cell identities that may potentiate or enable SARS-CoV-2 replication and tropism.

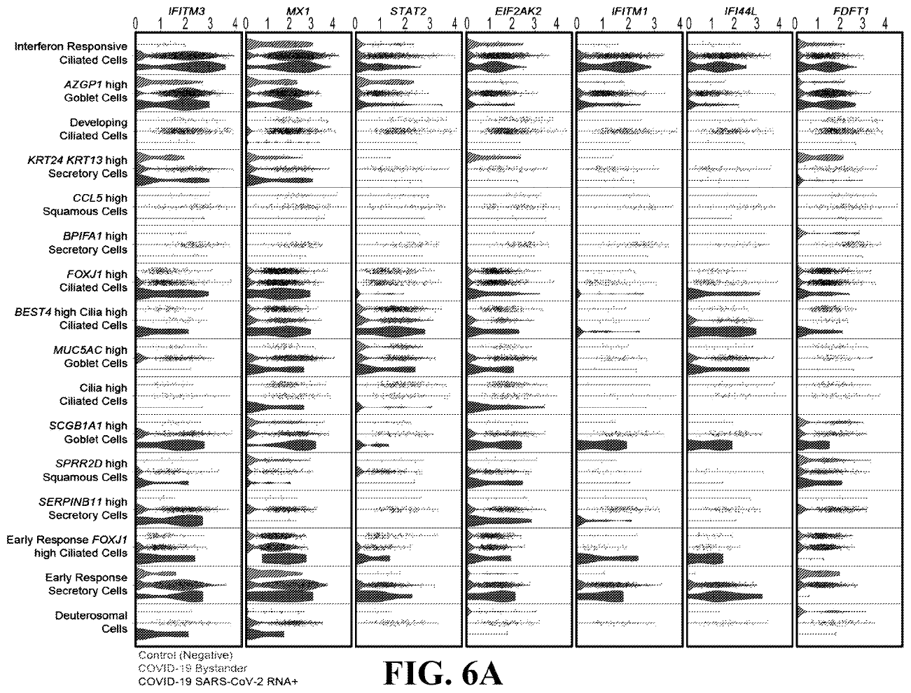

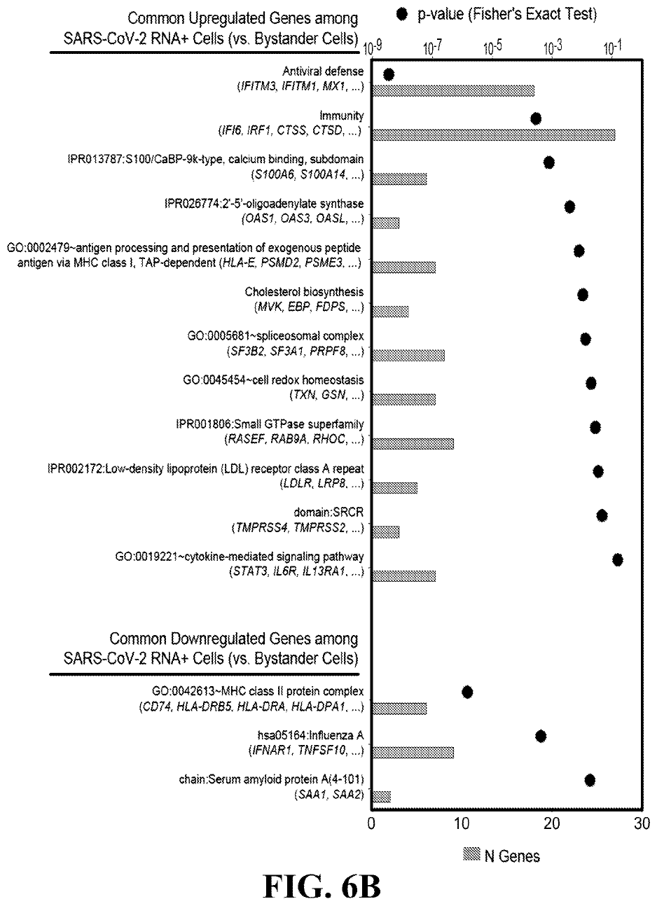

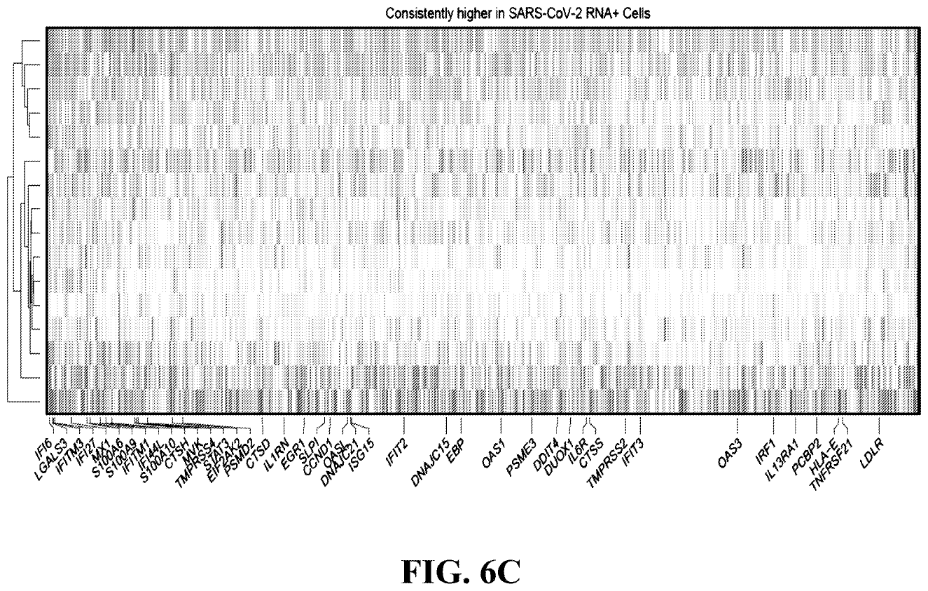

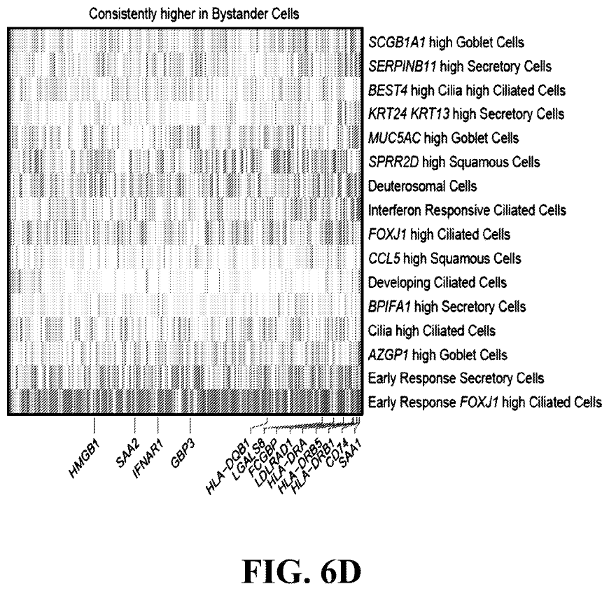

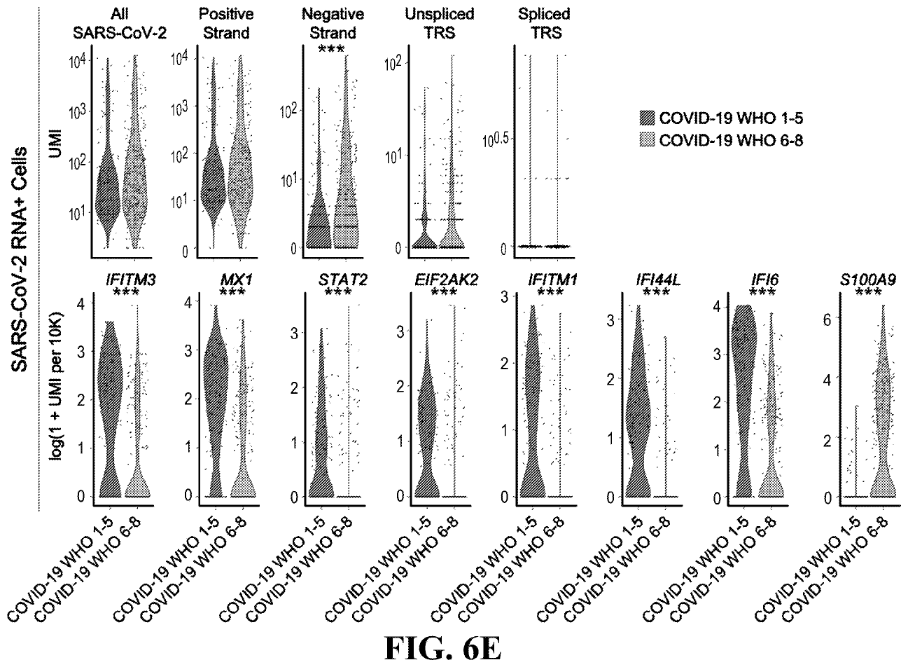

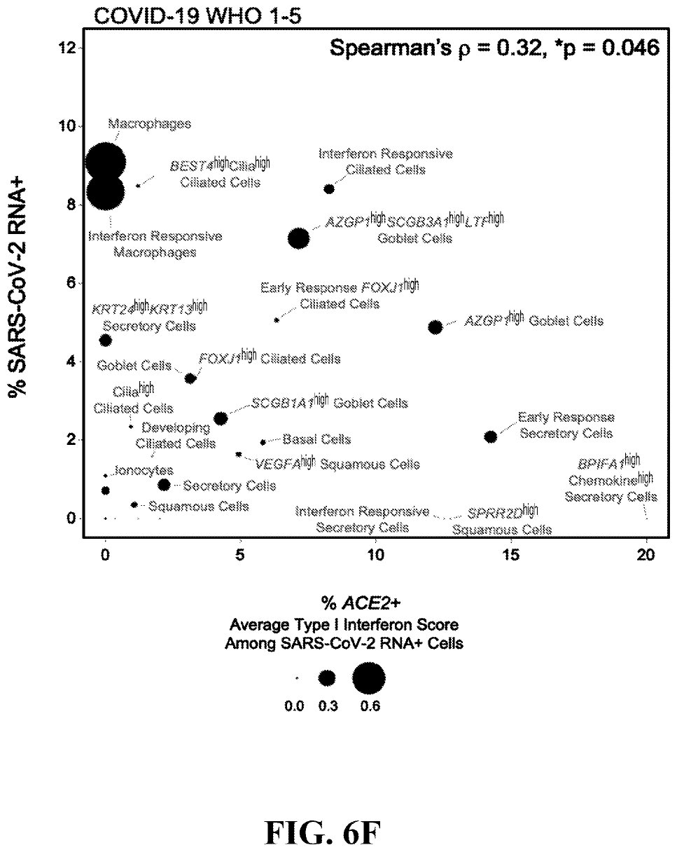

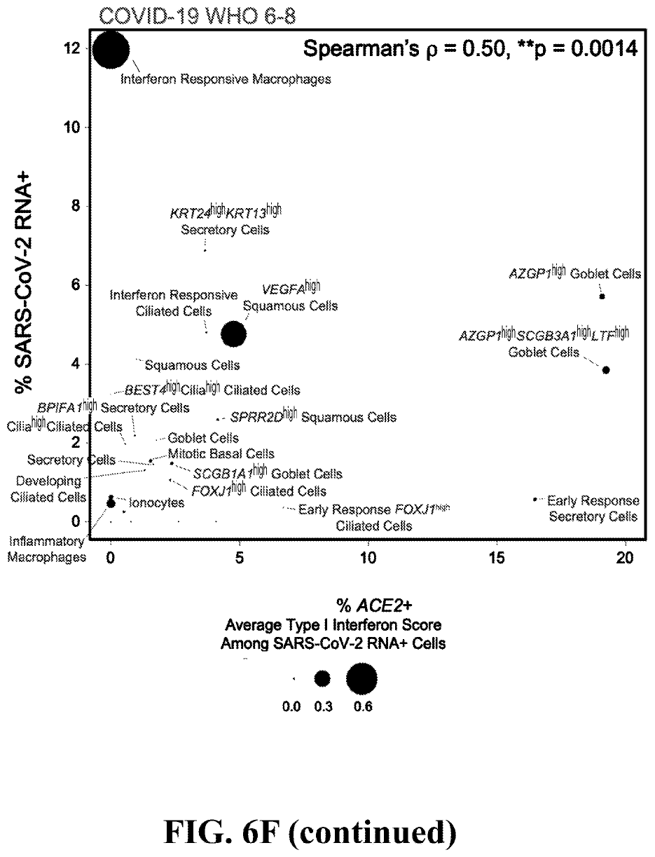

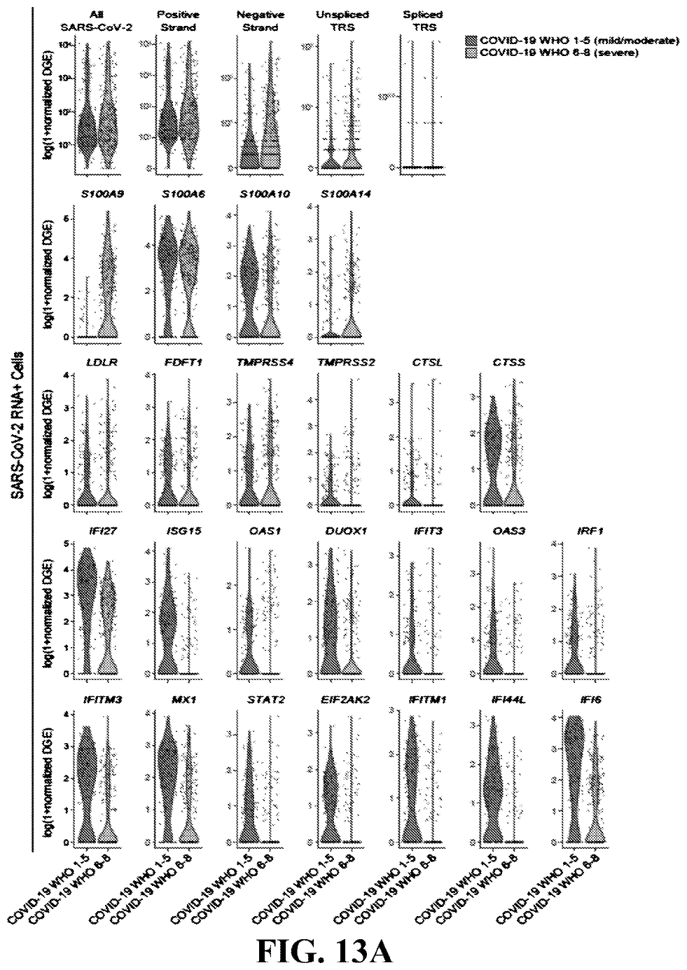

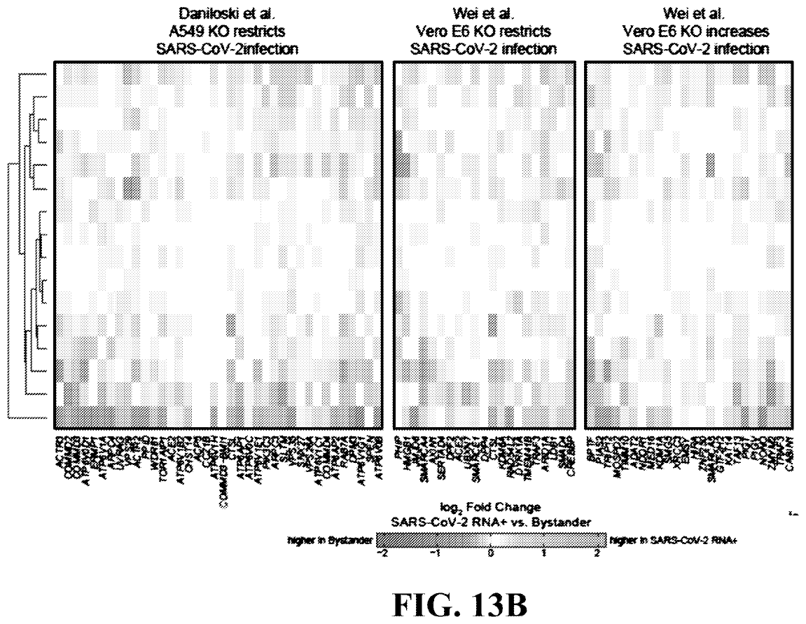

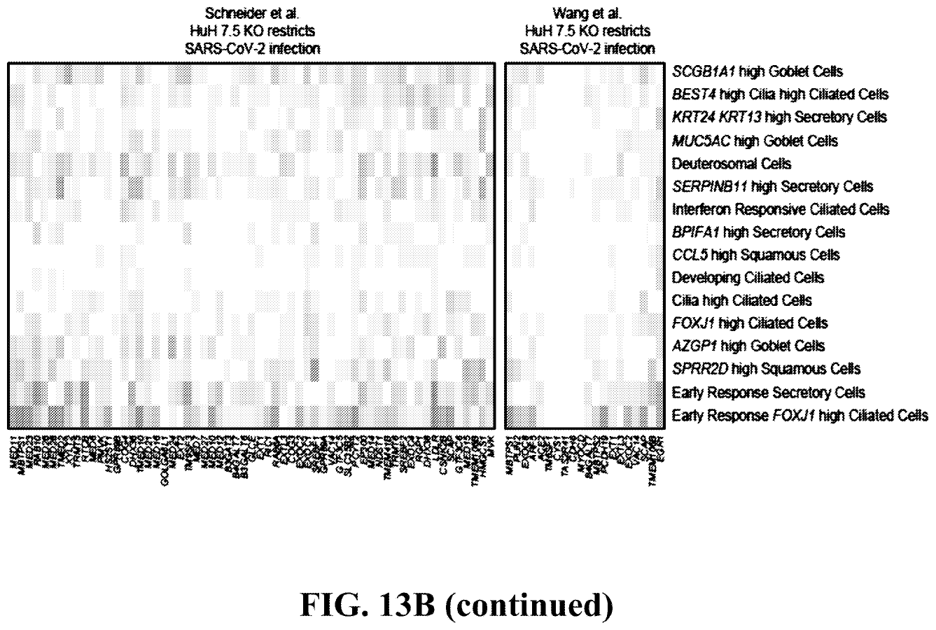

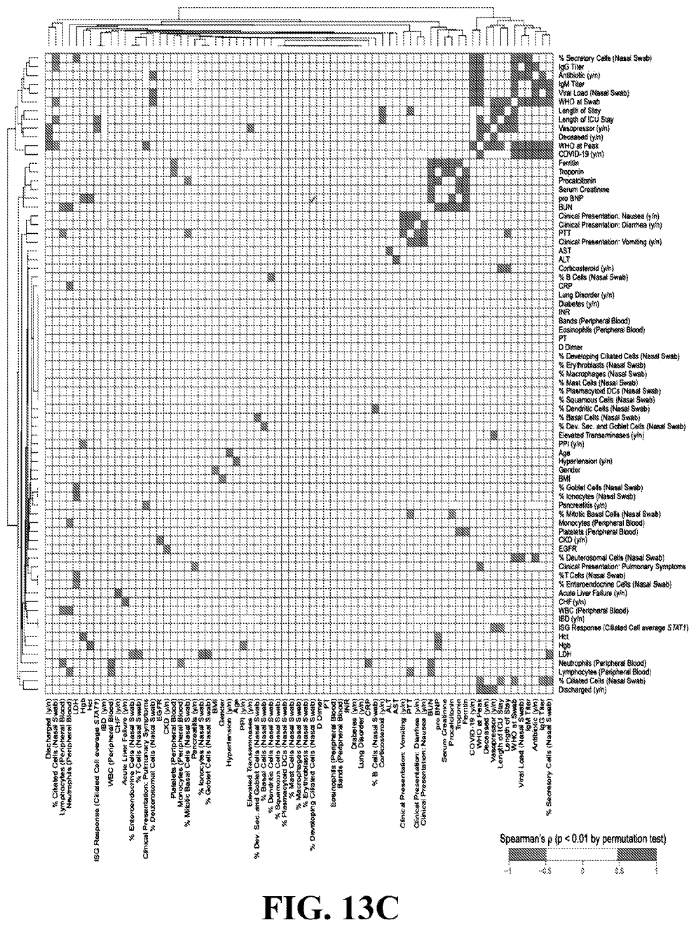

To control for variability among different SARS-CoV-2 RNA+ cell types and individuals, Applicants compared SARS-CoV-2 RNA+ cells to bystander cells of the same cell type and participant. Among cell types with at least 5 SARS-CoV-2 RNA+ cells, Applicants observed robust and specific transcriptional changes compared to both matched bystander cells as well as cells from healthy individuals (FIGS. 6A, 6B). Notably, many of the genes previously identified as increased within all cells from COVID-19 donors, e.g., anti-viral factors IFITM3, MXJ, IFI44L, and IRF1, were also upregulated among SARS-CoV-2 RNA+ cells compared to matched bystanders within multiple cell types. SARS-CoV-2 RNA+ cells from participants with mild/moderate COVID-19 showed stronger induction of anti-viral and interferon responsive pathways compared to those with severe COVID-19, despite equivalent abundances of cell-associated viral UMI (FIG. 13A). EIF2AK2, which encodes protein kinase R and drives host cell apoptosis following recognition of intracellular double-stranded RNA, was among the most reliably expressed and upregulated genes among SARS-CoV-2 RNA+ cells compared to matched bystanders across diverse cell types, suggesting rapid activation of this locus following intrinsic PAMP recognition of SARS-CoV-2 replication intermediates. Therefore, direct sensing of intracellular viral products amplifies interferon-responsive and anti-viral gene upregulation, though these pathways are also elevated within bystander cells. The majority of genes induced within SARS-CoV-2 RNA+ cells were shared across diverse cell types, suggesting a conserved anti-viral response, as well as common features that facilitate or restrict infection (FIG. 6B-6D, Table 5). SARS-CoV-2 RNA appeared to robustly stimulate expression of genes involved in anti-viral sensing and defense (e.g., MX1, IRF1, OAS1, OAS2), as well as genes involved in antigen presentation via MHC class I (FIG. 6C, Table 5). SARS-CoV-2 RNA+ cells expressed significantly higher abundances of multiple proteases involved in the cleavage of SARS-CoV-2 spike protein, a required step for viral entry (TMPRSS4, TMPRSS2, CTSS, CTSD). This suggests that within a given cell type, natural variations in the abundance of genes which support the viral life cycle partially account for which cells are successfully targeted by the virus. Among the core anti-viral/interferon-responsive gene sets induced within SARS-CoV-2 RNA+ cells, Applicants found repeated and robust upregulation of IFITM3 and IFITM1. Multiple studies have demonstrated that while these two interferon-inducible factors can disrupt viral release from endocytic compartments among a wide diversity of viral species, IFITMs can instead facilitate entry by human betacoronaviruses. Therefore, enrichment of these factors within presumptive infected cells may reflect viral hijacking of a conserved host anti-viral responsive pathway. Genes involved in cholesterol and lipid biosynthesis were also upregulated among SARS-CoV-2 RNA+ cells, including FDFT, MVK, FDPS, ACAT2, HMGCS1, all enzymes involved in the mevalonate synthesis pathway. In addition, SARS-CoV-2 RNA+ cells showed increased abundance of low-density lipoprotein receptors LDLR and LRP8 compared to matched bystanders. Intriguingly, various genes involved in cholesterol metabolism were recently identified as critical host factors for SARS-CoV-2 replication via CRISPR screens from multiple independent research groups^{56,57}. Further, these groups found that direct inhibition of cholesterol biosynthesis decreased SARS-CoV-2 (as well as coronavirus strains 299E and OC43) replication within cell lines, and suggest S-mediated entry relies on host cholesterol. Applicants queried the full collections of presumptive replication factors identified by four published CRISPR screens⁵⁶⁻⁵⁹, and found significant enrichment among SARS-CoV-2 RNA+ cells for RAB GTPases (e.g. RAB9A, RHOC, RASEF), vacuolar ATPase H+ pump subunits, as well as transcriptional modulators such as SPEN, SLTM, CREBBP, SMAD4 and EGR1 (FIG. 13B).

Finally, Applicants found multiple previously unappreciated genes implicated in susceptibility and response to SARS-CoV-2 infection, including S100/Calbindin genes such as S100A6, S100A4, and S100A9, which may directly play a role in leukocyte recruitment to infected cells. IFNAR1 was substantially increased in many bystander cells compared to both cells from SARS-CoV-2 negative participants as well as matched SARS-CoV-2 RNA+ cells (FIG. 6D). Blunting of interferon alpha signaling via downregulation of IFNAR1 within SARS-CoV-2 RNA+ cells may partially explain high levels of viral replication compared to neighboring cells. Moreover, this may represent a novel mechanism for interferon antagonism by SARS-CoV-2. Finally, bystander cells expressed significantly higher abundances of MHC-II molecules compared to SARS-CoV-2 RNA+ cells, including HLA-DQB1, HLA-DRB1, HLA-DRB5, HLA-DRA, and CD74.

Anti-viral factors were largely absent from presumptive virally infected cells in participants who developed severe COVID-19, despite equivalent abundances of cell-associated viral UMIs, and elevated UMIs/cell aligning to the viral negative strand (FIG. 6E, FIG. 13A). EIF2AK2, which encodes protein kinase R and drives host cell apoptosis following recognition of intracellular double-stranded RNA, is among the most reliably expressed and upregulated genes among SARS-CoV-2 RNA+ cells compared to matched bystanders across diverse cell types, suggesting rapid activation of this gene following intrinsic PAMP recognition of SARS-CoV-2 replication intermediates (Krahling et al., (2009). Severe Acute Respiratory Syndrome Coronavirus Triggers Apoptosis via Protein Kinase R but Is Resistant to Its Antiviral Activity. J. Virol.). Neither EIF2AK2 nor IFN-responsive transcription factors such as STAT1 and STAT2 were expressed within SARS-CoV-2 RNA+ cells from participants who developed severe COVID-19 (FIG. 6E). This suggests that direct sensing of intracellular viral products may amplify IFN-responsive and anti-viral gene upregulation, though these pathways are only induced among SARS-CoV-2 RNA+ cells from participants with mild/moderate COVID-19 (FIG. 6F). Together, this suggests a failure of the intrinsic immune response to viral infection among nasal epithelial cells in individuals who develop severe COVID-19.

### Example 7—Discussion

Here, Applicants have created a comprehensive map of SARS-CoV-2 infection of the human nasopharynx using scRNA-seq, and identified tissue correlates of protection and disease severity within a large human cohort. By linking a detailed census of cell types and states across disease outcomes, Applicants begin to untangle the myriad factors that underlie restriction of viral infection to the upper respiratory tract vs. expansion to the lower airways and lung parenchyma or support the development of severe lower respiratory tract disease (FIG. 13C). This study defines major compositional differences in the nasal epithelia during COVID-19 and directly relates these to NP viral load, cellular tropism, and cell-intrinsic responses to SARS-CoV-2. Further, Applicants identify marked variability in the induction of anti-viral gene expression that is associated with peak disease severity and may precede development of severe respiratory damage. Applicants find that anti-viral gene expression is profoundly blunted in cells isolated from individuals who develop severe disease, even in cells containing SARS-CoV-2 RNA.

First, Applicants find that mature ciliated cells decline dramatically within the nasopharynx of COVID-19 samples, directly correlated with the tissue abundance of SARS-CoV-2 RNA at the time of sampling. Conversely, secretory cell populations expand among samples with high viral loads, which potentially represents a conserved response for epithelial repopulation of lost mature ciliated cells through a recently identified mechanism of secretory/goblet trans-differentiation, using deuterosomal cells as intermediates. Accordingly, deuterosomal cells and immature/developing ciliated cells were dramatically expanded among COVID-19 samples, suggesting interdependence between each of these compartments in maintaining epithelial homeostasis during viral challenge. Further work is required to understand how this process relates to epithelial responses in other common upper respiratory viral infections and inflammatory states. Broadly, SARS-CoV-2 infection induced dramatic increases in the diversity of epithelial cell types, both with respect to shifted compositional balance among major cell identities, and also via expansion of specialized secretory and goblet cell subsets, including a subset termed KRT13 KRT24 high Secretory Cells, which closely match the recently-identified KRT13 “hillock” cell, previously associated with epithelial regions experiencing rapid cellular turnover and inflammation⁴⁹. Other specialized subsets of secretory and goblet cells, such as Early Response Secretory Cells, AZGP1 high Goblet Cells, and SCGB1A1 high Goblet Cells are expanded among COVID-19 participants, however, are found within discrete subsets of individuals and are not homogenous across the disease cohorts Applicants sample here. Indeed, heterogeneous responses in the epithelial compartment between individuals with COVID-19 underscores the need for larger cohort studies, with a focus on longitudinal responses following initial infection.

Beyond compositional changes during COVID-19, this study found that individuals who developed severe disease exhibited profoundly blunted anti-viral responses and diminished expression of interferon-responsive genes compared to individuals with milder courses. This effect was observed among diverse cell types, including those thought to represent direct targets of viral infection, such as ciliated cells and secretory cells, and also bystanders and co-resident immune cells. Notably, individuals with severe COVID-19 disease had equivalent or even elevated levels of nasal SARS-CoV-2 RNA at the time of sampling, and contained expanded inflammatory and type II-interferon responsive macrophages compared to mild/moderate cases. Surprisingly, even among mild cases with robust interferon stimulated gene expression, Applicants found little to no type I/III interferon transcription amongst any recovered cell types. In a related study mapping the nasal epithelium during influenza infection, the authors found extensive upregulation of IFNA, IFNB1, and IFNL1-3 within ciliated cells and goblet cells, both highlighting the capacity of superficial nasal epithelial cells to secrete local interferons during viral infection, but also the technical capacity of the scRNA-seq platform used in both studies to capture interferon mRNA. The precise source and signal which motivates a broad anti-viral response among mild COVID-19 cases in this study remains unknown, and may originate from immune cells contained deeper within the respiratory mucosa (therefore inaccessible through the superficial sampling used here), or may derive from direct PAMP/DAMP sensing or alternative inflammatory signals. Indeed, published peripheral immune studies comparing mild and severe COVID-19 also observe diminished type I and type III interferon abundances, and note restricted interferon stimulated gene expression among circulating immune cells^{17,18}. The close association between disease severity and weak anti-viral gene expression among nasal epithelial cells is also intriguing given recent observations of inborn defects in TLR3, IRF7, IRF9, and IFNAR1 or direct antibody-mediated neutralization of secreted type I interferons within individuals who develop severe COVID-19³²⁻³⁴. Even among cells containing SARS-CoV-2 RNA, individuals who developed severe disease failed to induce expression of classic anti-viral factors including MX1, IFITM1, ISG15, which were all robustly associated with intracellular viral RNA within mild/moderate cases. Further, Applicants found lower nasal viral loads were associated with elevated detection of tissue plasmacytoid DCs, suggesting diminished or delayed recruitment of these cells may partially explain how local viral replication proceeds to such high abundances. These findings strongly suggest severe infection can arise in the setting of an intrinsic impairment of epithelial anti-viral immunity. Further, human betacoronaviruses including MERS, SARS-CoV, and SARS-CoV-2 all exhibit multiple strategies to avoid triggering pattern recognition receptor pathways, including degradation of host mRNA within infected cells, sequestration of viral replication intermediates (e.g., double stranded RNA) from host sensors, and direct inhibition of immune effector molecules, thereby leading to diminished induction of anti-viral pathways and blunted autocrine and paracrine interferon signaling. Applicants surmise that the combined effects of a viral strain with naturally poor interferon induction in a host with intrinsic defects in immune or epithelial anti-viral responses drives prolonged viral replication in the upper airway, which eventually leads to immunopathology characteristic of severe COVID-19.

Critically, this work does not address the dynamics of nasal epithelial anti-viral responses during SARS-CoV-2 infection, nor does it directly relate failed intrinsic epithelial immunity in the nasopharynx to potential interferon or anti-viral responses in the lung or distal airways. Indeed, related work suggests type III interferons are present in the lungs, but not the nasopharynx, during SARS-CoV-2 infection, and may contribute to tissue damage late in disease course⁶⁰. Further, as the individuals in this cohort were intentionally sampled as early within their disease course as possible, and the majority have elevated viral levels within their nasopharynx, the findings have an unclear relation to the tissue response during hyper-inflammatory “late” stages of COVID-19. However, among individuals who develop severe COVID-19, Applicants observe unique recruitment of highly inflammatory macrophages that represent the major tissue sources of proinflammatory cytokines including IL1B, TNF, CXCL8, CCL2, CCL3 and CXCL9/10/11—of likely relation to the immune dysregulation characterized by elevation of the same factors in the periphery in late, severe disease. In addition, Applicants note specific upregulation of alarmins S100A8 S100A9 (which together form TLR4 and RAGE ligand calprotectin) among epithelial cells in severe COVID-19 compared to mild and control counterparts, and even higher expression of S100A9 within SARS-CoV-2 RNA+ cells from those same individuals. A recent study identified these as potential biomarkers of severe COVID-19, and proposed that these factors directly drive excessive inflammation and precede the massive cytokine release characteristic of late disease. This work suggests that severe COVID-19-specific expression of calprotectin may originate instead within the virally-infected nasal epithelia, and suggests that further work to understand the epithelial cell regulation of S100A8/A9 gene expression may help clarify maladaptive responses to SARS-CoV-2 infection.

Finally, Applicants provide a direct investigation into the host factors that enable or restrict SARS-CoV-2 replication within epithelial cells in vivo. Here, Applicants recapitulate expected “hits” based on well-described host factors involved in viral replication, e.g., TMPRSS2, TMPRSS4 enrichment among presumptive virally infected cells. Applicants similarly observed expression of anti-viral genes which were globally enriched among cells from mild/moderate COVID-19 participants, with even higher expression among the viral RNA+ cells themselves. In accordance with previous studies into the nasal epithelial response to influenza infection, Applicants observed bystander epithelial cell upregulation of both MHC-I and MHC-II family genes, however found that SARS-CoV-2 RNA+ cells only expressed MHC-I, and uniformly downregulated MHC-II genes compared to matched bystanders. To Applicants' knowledge, downregulation of host cell pathways for antigen presentation by coronaviruses has not been previously described. A recent study found that CIITA and CD74 can intrinsically block entry of a range of viruses (including SARS-CoV-2) via endosomal sequestration, and therefore cells that upregulate these (and other) components of MHC-II machinery may naturally restrict viral entry.

Together, this work demonstrates that many of the factors that determine the clinical trajectory following SARS-CoV-2 infection stem from initial host-viral encounters in the nasopharyngeal epithelium. Further, it implies that dysregulated tissue immunity may be subverted by focusing preventative or therapeutic interventions early within the nasopharynx, thereby bolstering anti-viral responses and curbing pathological inflammatory signaling prior to development of severe respiratory dysfunction or systemic disease.

### Example 8—Methods

Study Participants and Design—Subjects 18 years and older were recruited from the University of Mississippi Medical Center (UMMC) (Jackson, Mississippi) between April 2020 and September 2020. All patients were enrolled in the prospective study at UMMC, which included patients with COVID-19 who were inpatient hospitalized as well as non-COVID-19 (control) who were outpatient and seen at UMMC Acute Respiratory Clinic or UMMC GI Endoscopy. Inclusion criteria for COVID-19 participants included fever, cough, sore throat and/or shortness of breath with presumed diagnosis of COVID-19 upper respiratory tract infection. The patients all weighed 110 lbs or greater. Non-COVID-19 (control) participants all had a negative SARS-CoV-2 test, weighed 110 pounds or greater, and were seen in either GI Endoscopy or UMMC Acute Respiratory Clinic. Exclusion criteria for both cohorts included a history of blood transfusion within 4 weeks and subjects who could not be assigned a definitive COVID-19 diagnosis from either nucleic acid testing or Chest CT imaging. For the nasopharyngeal (NP) samples, 38 individuals with COVID-19 were included, both male (n=20) and female (n=18). 21 of the participants were non-COVID-19 (control)—11 identified as male, 10 as female. The median age of COVID-19 participants was 56.5 years old; the median age of Control participants was 62 years old. Among hospitalized participants, samples were collected between Day 1 to Day 3 of hospitalization. The Institutional Review Board approved the study, and all subjects provided written informed consent, or their legally authorized representative provided it on their behalf. Research samples were collected from volunteers in the form of nasal swabs. A healthcare provider collected the nasopharyngeal sample using two cotton swabs. COVID-19 participants were classified according to the 8-level ordinal scale proposed by the WHO representing severity and level of respiratory support required.

Sample Collection and Biobanking—Nasopharyngeal samples were collected by trained healthcare provider using FLOQSwabs (Copan flocked swabs) following the manufacturer's instructions. Collectors would don personal protective equipment (PPE), including a gown, non-sterile gloves, a protective N95 mask, a bouffant, and a face shield. The patient's head was then tilted back slightly, and the swab inserted along the nasal septum, above the floor of the nasal passage to the nasopharynx until slight resistance was felt. The swab was then left in place for several seconds to absorb secretions and slowly removed while rotating swab. A second swab was then completed in the other nares. The swabs were then placed into a cryogenic vial with 900 μL of heat inactivated fetal bovine serum (FBS) and 100 μL of dimethyl sulfoxide (DMSO). The vials were then placed into a Thermo Scientific Mr. Frosty Freezing Container for optimal cell preservation. The Mr. Frosty containing the vials was then placed in cooler with dry ice for transportation from patient area to laboratory for processing. Once in the laboratory, the Mr. Frosty was placed into the −80° C. Freezer overnight and then on the next day, the vials were moved to the liquid nitrogen storage container.

Dissociation and Collection of Viable Single Cells from Nasal Swabs—Swabs in freezing media (90% FBS/10% DMSO) were stored in liquid nitrogen until immediately prior to dissociation. A detailed sample protocol can be found here: protocols.io/view/human-nasopharyngeal-swab-processing-for-viable-si-bjhkkj4w.html. This approach ensures that all cells and cellular material from the nasal swab (whether directly attached to the nasal swab, or released during the washing and digestion process), are exposed first to DTT for 15 minutes, followed by an Accutase digestion for 30 minutes. Briefly, nasal swabs in freezing media were thawed, and each swab was rinsed in RPMI before incubation in 1 mL RPMI/10 mM DTT (Sigma) for 15 minutes at 37° C. with agitation. Next, the nasal swab was incubated in 1 mL Accutase (Sigma) for 30 minutes at 37° C. with agitation. The 1 mL RPMI/10 mM DTT from the nasal swab incubation was centrifuged at 400 g for 5 minutes at 4° C. to pellet cells, the supernatant was discarded, and the cell pellet was resuspended in 1 mL Accutase and incubated for 30 minutes at 37° C. with agitation. The original cryovial containing the freezing media and the original swab washings were combined and centrifuged at 400 g for 5 minutes at 4° C. The cell pellet was then resuspended in RPMI/10 mM DTT, and incubated for 15 minutes at 37° C. with agitation, centrifuged as above, the supernatant was aspirated, and the cell pellet was resuspended in 1 mL Accutase, and incubated for 30 minutes at 37° C. with agitation. All cells were combined following Accutase digestion and filtered using a 70 μm nylon strainer. The filter and swab were washed with RPMI/10% FBS/4 mM EDTA, and all washings combined. Dissociated, filtered cells were centrifuged at 400 g for 10 minutes at 4° C., and resuspended in 200 μL RPMI/10% FBS for counting. Cells were diluted to 20,000 cells in 200 μL for scRNA-seq. For the majority of swabs, fewer than 20,000 cells total were recovered. In these instances, all cells were input into scRNA-seq.

scRNA-seq—Seq-Well S³ was run as previously described⁴⁴⁻⁴⁶. Briefly, a maximum of 20,000 single cells were deposited onto Seq-Well arrays preloaded with a single barcoded mRNA capture bead per well. Cells were allowed to settle by gravity into wells for 10 minutes, after which the arrays were washed with PBS and RPMI, and sealed with a semi-permeable membrane for 30 minutes, and incubated in lysis buffer (5 M guanidinium thiocyanate/1 mM EDTA/1% BME/0.5% sarkosyl) for 20 minutes. Arrays were then incubated in a hybridization buffer (2M NaCl/8% v/v PEG8000) for 40 minutes, and then the beads were removed from the arrays and collected in 1.5 mL tubes in wash buffer (2M NaCl/3 mM MgCl₂/20 mM Tris-HCl/8% v/v PEG8000). Beads were resuspended in a reverse transcription master mix, and reverse transcription, exonuclease digestion, second strand synthesis, and whole transcriptome amplification were carried out as previously described. Libraries were generated using Illumina Nextera XT Library Prep Kits and sequenced on NextSeq 500/550 High Output v2.5 kits to an average depth of 180 million aligned reads per array: read 1: 21 (cell barcode, UMI), read 2: 50 (digital gene expression), index 1: 8 (N700 barcode).

Data Preprocessing and Quality Control—Pooled libraries were demultiplexed using bcl2fastq (v2.17.1.14) with default settings (mask_short_adapter_reads 10, minimum_trimmed_read_length 10, implemented using Cumulus, snapshot 4, cumulus.readthedocs.io/en/stable/bcl2fastq.html). Libraries were aligned using STAR within the Drop-Seq Computational Protocol (github.com/broadinstitute/Drop-seq) and implemented on Cumulus (cumulus.readthedocs.io/en/latest/drop_seq.html, snapshot 9, default parameters). A custom reference was created by combining human GRCh38 (from CellRanger version 3.0.0, Ensembl 93) and SARS-CoV-2 RNA genomes. The SARS-CoV-2 viral sequence and GTF are as described in Kim et al. 2020 (github.com/hyeshik/sars-cov-2-transcriptome, BetaCov/South Korea/KCDC03/2020 based on NC_045512.2). The GTF includes all CDS regions (as of this annotation of the transcriptome, the CDS regions completely cover the RNA genome without overlapping segments), and regions were added to describe the 5′ UTR (“SARSCoV2_5prime”), the 3′ UTR (“SARSCoV2_3prime”), and reads aligning to anywhere within the Negative Strand (“SARSCoV2_NegStrand”). Trailing A's at the 3′ end of the virus were excluded from the SARS-CoV-2 FASTA, as these were found to drive spurious viral alignment in pre-COVID19 samples. Finally, additional small sequences were appended to the FASTA and GTF that differentiate reads that align to the 70-nucleotide region around the viral TRS sequence—either across the intact, unspliced genomic sequences (e.g., named “SARSCoV2_Unspliced_S” or “SARSCoV2_Unspliced_Leader”) or various spliced RNA species (e.g., “SARSCoV2_Spliced_Leader_TRS_S”), see schematics in FIGS. 12K, 12L. Alignment references were tested against a diverse set of pre-COVID-19 samples and in vitro SARS-CoV-2 infected human bronchial epithelial cultures (Ravindra et al.) to confirm specificity of viral aligning reads (data not shown). Aligned cell-by-gene matrices were merged across all study participants, and cells were filtered to eliminate barcodes with fewer than 200 UMI, 150 unique genes, and greater than 50% mitochondrial reads (cutoffs determined by distributions of reads across cells, see FIG. 7C). Of the 61 nasal swabs thawed and processed, 3 contained no high-quality cell barcodes after sequencing (NB: these samples contained <5,000 viable cells prior to Seq-Well array loading). This resulted in a final dataset of 32,871 genes and 32,588 cells across 58 study participants (35 COVID-19 individuals, 21 control individuals, 2 COVID-19 convalescent individuals). Preprocessing, alignment, and data filtering was applied equivalently to samples from the fresh vs. frozen cohort. For analysis of RNA velocity, Applicants also recovered both exonic and intronic alignment information using DropEst (Cumulus (cumulus.readthedocs.io/en/latest/drop_seq.html, snapshot 9, dropest_velocyto true, run_dropest true).

Cell Clustering and Annotation—Dimensionality reduction, cell clustering and differential gene analysis were all achieved using the Seurat (v3.1.5) package in R programming language (v3.0.2). Dimensionality reduction was carried out by running principal components analysis over the 3,483 most variable genes with dispersion >0.8 (tested over a range of dispersion >0.7 to dispersion >1.2; dispersion >0.8 was determined as optimal based on number of variable genes, and general stability of clustering results across these cutoffs was confirmed). Only variable genes from human transcripts were considered for dimensionality reduction and clustering. Using the Jackstraw function within Seurat, Applicants selected the first 36 principal components that described the majority of variance within the dataset, and used these for defining a nearest neighbor graph and Uniform Manifold Approximation and Projection (UMAP) plot. Cells were clustered using Louvain clustering, and the resolution parameter was chosen by maximizing the average silhouette score across all clusters. Differentially expressed genes between each cluster and all other cells were calculated using the FindAllMarkers function, test.use set to “bimod”. Clusters were merged if they failed to contain sets of significantly differentially expressed genes. Applicants proceeded iteratively through each cluster and subcluster until “terminal” cell subsets/cell states were identified—Applicants defined “terminal” cell states as those for whom principal components analysis and Louvain clustering did not confidently identify additional sub-states, as measured by abundance of differentially expressed genes between potential clusters. For visualization in FIGS. 2, 3, and FIG. 9, Applicants pooled all cells determined to be of epithelial origin, and using the methods for dimensionality reduction as above (dispersion cutoff >1, 30 principal components). Applicants applied similar approaches for immune cell types, including iterative subclustering to resolve and annotate all constituent cells types and subtypes, and combined all immune cells for visualization purposes in FIG. 10. Cell cycle scoring utilized gene lists from Tirosh et al. Gene module scores were calculated using the AddModuleScore function within Seurat.

RNA Velocity and Pseudotemporal Ordering of Epithelial Cells—RNA velocity was modeled using the scVelo package, version 0.2.3. Using cluster annotations previously assigned from iterative clustering in Seurat, cells from epithelial cell types were pre-processed according to the scVelo pipeline: genes were normalized using default parameters (pp.filter_and_normalize), principal components and nearest neighbors in PCA space were calculated (using defaults of 30 PCs, 30 nearest neighbors), and the first and second order moments of nearest neighbors were computed, which are used as inputs into velocity estimates (pp.moments). RNA velocity was estimated using the scVelo tool tl.recover_dynamics with default input parameters, which maps the full splicing kinetics for all genes and tl.velocity, with mode=‘dynamical’. Top velocity transition “driver” genes were identified by high “fit_likelihood” parameters from the dynamical model, and are used for visualization in FIG. 9G. The same approaches were used for modeling RNA velocity among only Ciliated Cells (FIG. 2H-2K), Basal, Secretory, and Goblet Cells (FIG. 2L-2O), and only COVID-19 or only Control cells (FIG. 3A). For RNA velocity analysis of Ciliated Cells or Basal, Secretory and Goblet Cells, the velocity pseudotime was calculated using the tl.velocity_pseudotime function with default settings.

Metagenomic Classification of Reads from Single-Cell RNA-Seq—To identify co-detected microbial taxa present in the cell-associated or ambient RNA of nasopharyngeal swabs, Applicants used the Kraken2 software implemented using the Broad Institute viral-ngs pipelines on Terra (github.com/broadinstitute/viral-pipelines/tree/master). A previously-published reference database included Human, archaea, bacteria, plasmid, viral, fungi, and protozoa species and was constructed on May 5, 2020, therefore included sequences belonging to the novel SARS-CoV-2 virus. Inputs to Kraken2 were: kraken2_db_tgz=“gs://pathogen-public-dbs/v1/kraken2-broad-20200505.tar.zst”, krona_taxonomy_db_kraken2_tgz=“gs://pathogen-public-dbs/v1/krona.taxonomy-20200505.tab.zst”, ncbi_taxdump_tgz=“gs://pathogen-public-dbs/v1/taxdump-20200505.tar.gz”, trim_clip_db=“gs://pathogen-public-dbs/v0/contaminants.clip_db.fasta” and spikein_db=“gs.//pathogen-public-dbs/v0/ERCC_96_nopolyA.fasta”. Species with fewer than 5 reads were considered spurious and excluded.

Correction for Ambient Viral RNA—Single-cell data from high-throughput single-cell RNA-seq platforms frequently experience low-levels of non-specific RNA assigned to cell barcodes that does not represent true cell-derived transcriptomic material, but rather contamination from the ambient pool of RNA. To safeguard against spurious assignment of SARS-CoV-2 RNA to cells without true intracellular viral material, i.e., viral RNA non-specifically picked up from the microenvironment as a component of ambient RNA contamination, Applicants employed the following corrections and statistical tests to correct for ambient viral RNA and enable confident assignments for SARS-CoV-2 RNA+ cells. Similar to approaches previously described, Applicants tested whether the abundance of viral RNA within a given single cell was significantly higher than expected by chance given the estimate of ambient RNA contaminating that cell, as well as the proportion of viral RNA of the total ambient RNA pool. First, this required modeling and estimating the ambient RNA fraction associated with each individual swab. Here, Applicants employed CellBender (github.com/broadinstitute/CellBender), a software package built to learn the ambient RNA profile and provide an ambient RNA-corrected output. Input UMI count matrices contained the top 10,000 cell barcodes, therefore including at least 70% cell barcodes sampling the ambient RNA of low-quality cell pool. CellBender's remove-background function was run with default parameters and --fpr 0.01 --expected-cells 500 --low-count-threshold 5. Using the corrected output from each sample's count matrix following CellBender, Applicants calculated the proportion of ambient contamination per high-quality cell by comparing to the single-cell's transcriptome pre-correction, and summed all UMI from background/low-quality cell barcodes to recover an estimate of the total ambient pool. Next, Applicants tested whether the abundance of viral RNA in a given single cell was significantly above the null abundance given the ambient RNA characteristics using an exact binomial test (implemented in R (binom.test):

$$P ( x ) = \frac{n !}{( n - x ) ! x !} {p}^{x} {q}^{n - x}$$

where n=SARS-CoV-2 UMI per cell, x=total UMI per cell
p=(ambient fraction per cell)*(SARS-CoV-2 UMI fraction of all ambient UMI), and q=1−p

P-values were FDR-corrected within sample, and cells whose SARS-CoV-2 UMI abundance with FDR <0.01 were considered “SARS-CoV-2 RNA+”.

Differential Expression by Cohort, Cell Type, or Viral RNA Status—To compare gene expression between cells from distinct donor cohorts Applicants employed a negative binomial generalized linear model. Cells from each cell type belonging to either COVID-19 WHO 1-5 (mild/moderate), COVID-19 WHO 6-8 (severe), or Control WHO 0 were compared in a pairwise manner, implemented using the Seurat FindAllMarkers function. Applicants considered genes as differentially expressed with an FDR-adjusted p value <0.001 and log fold change >0.25. To compare gene expression between SARS-CoV-2 RNA+ cells and bystander cells (from COVID-19 participants, but without intracellular viral RNA) Applicants again used a negative binomial generalized linear model, but instead implemented using DESeq2. Applicants only tested cell types containing at least 15 SARS-CoV-2 RNA+ cells, and for each cell type, Applicants restricted the bystander cells to the same participants as the SARS-CoV-2 RNA+ cells. Next, given the large discrepancies in cell number between SARS-CoV-2 RNA+ and bystander groups among most cell types, Applicants randomly sub-sampled the bystander cells to at most 4× the number of SARS-CoV-2 RNA+ cells. Further, Applicants selected bystander cell subsets that matched the cell quality distribution of the SARS-CoV-2 RNA+ cells, based on binned deciles of UMI/Cell. DESeq2 was run with default parameters and test=“Wald”. Gene ontology analysis was run using the Database for Annotation, Visualization, and Integrated Discovery (DAVID). Gene set enrichment analysis (GSEA) was completed using the R package fgsea over genes ranked by average log foldchange expression between each cohort, including all genes with an average expression >0.5 UMI within each respective cell type. Gene lists corresponding to “Shared IFN Response”, “Type I IFN Specific Response” and “Type II IFN Specific Response” are derived from previously-published population RNA-seq data from nasal epithelial basal cells treated in vitro with 0.1 ng/mL—10 ng/mL IFNA or IFNG for 12 hours. Module scores were calculated using the Seurat function AddModuleScore with default inputs.

Statistical Testing—All statistical tests were implemented either in R (v4.0.2) or Prism (v6) software. Comparisons between cell type proportions by cohort were tested using a Kruskal-Wallis test and Bonferroni-correction, implemented in R using the kruskal.test, and p.adjust functions. Post-tests for between-group pairwise comparisons used Dunn's test. Spearman correlation was used where appropriate, implemented using the cor.test function in R. All testing for differential expression was implemented in R using either Seurat, scVelo, or DESeq2, and all results were FDR-corrected as noted in specific Methods sections. P-values, n, and all summary statistics are provided either in the results section, figure legends, figure panels, or tables.

Data and Code Availability—Prism (v6), R (v4.0.2) packages ggplot2 (v3.3.2), Seurat (v3.2.2), ComplexHeatmap (v2.7.3), and Circlize (0.4.11), fgsea (v.1.16.0) and Python (v3.8.3) package scVelo (v0.3.0) were used for visualization.

### REFERENCES

1. Meyerowitz, E. A., Richterman, A., Gandhi, R. T. & Sax, P. E. Transmission of SARS-CoV-2: A Review of Viral, Host, and Environmental Factors. Ann. Intern. Med (2021) doi:10.7326/m20-5008.
2. Fears, A. C. et al. Persistence of Severe Acute Respiratory Syndrome Coronavirus 2 in Aerosol Suspensions. Emerg. Infect. Dis. (2020) doi:10.3201/eid2609.201806.
3. Pan, Y., Zhang, D., Yang, P., Poon, L. L. M. & Wang, Q. Viral load of SARS-CoV-2 in clinical samples. The Lancet Infectious Diseases (2020) doi:10.1016/S1473-3099(20)30113-4.
4. Sanche, S. et al. RESEARCH High Contagiousness and Rapid Spread of Severe Acute Respiratory Syndrome Coronavirus 2. Emerg. Infect. Dis. (2020) doi:10.3201/eid2607.200282.
5. Arons, M. M. et al. Presymptomatic SARS-CoV-2 Infections and Transmission in a Skilled Nursing Facility. N. Engl. J. Med (2020) doi:10.1056/nejmoa2008457.
6. Wang, Y. et al. Clinical outcome of 55 asymptomatic cases at the time of hospital admission infected with SARS-Coronavirus-2 in Shenzhen, China. J. Infect. Dis. (2020) doi:10.1093/infdis/jiaa119.
7. Sakurai, A. et al. Natural History of Asymptomatic SARS-CoV-2 Infection. N. Engl. J Med (2020) doi:10.1056/nejmc2013020.
8. Guan, W. et al. Clinical Characteristics of Coronavirus Disease 2019 in China. N. Engl. J Med (2020) doi:10.1056/nejmoa2002032.
9. Huang, C. et al. Clinical features of patients infected with 2019 novel coronavirus in Wuhan, China. Lancet (2020) doi:10.1016/S0140-6736(20)30183-5.
10. Chan, J. F. W. et al. Genomic characterization of the 2019 novel human-pathogenic coronavirus isolated from a patient with atypical pneumonia after visiting Wuhan. Emerg. Microbes Infect. (2020) doi:10.1080/22221751.2020.1719902.
11. Frieman, M. & Baric, R. Mechanisms of Severe Acute Respiratory Syndrome Pathogenesis and Innate Immunomodulation. Microbiol. Mol. Biol. Rev. (2008) doi:10.1128/mmbr.00015-08.
12. Harrison, A. G., Lin, T. & Wang, P. Mechanisms of SARS-CoV-2 Transmission and Pathogenesis. Trends in Immunology (2020) doi:10.1016/j.it.2020.10.004.
13. Lucas, C. et al. Longitudinal analyses reveal immunological misfiring in severe COVID-19. Nature (2020) doi:10.1038/s41586-020-2588-y.
14. Mathew, D. et al. Deep immune profiling of COVID-19 patients reveals distinct immunotypes with therapeutic implications. Science (80-.). (2020) doi:10.1.126/SCIENCE.ABC8511.
15. Schulte-Schrepping, J. et al. Severe COVID-19 Is Marked by a Dysregulated Myeloid Cell Compartment. Cell (2020) doi:10.1016/j.cell.2020.08.001.
16. Su, Y. et al. Multi-Omics Resolves a Sharp Disease-State Shift between Mild and Moderate COVID-19. Cell (2020) doi:10.1016/j.cell.2020.10.037.
17. Galani, I. E. et al. Untuned antiviral immunity in COVID-19 revealed by temporal type I/III interferon patterns and flu comparison. Nat. Immunol. (2021) doi:10.1038/s41590-020-00840-x.
18. Hadjadj, J. et al. Impaired type I interferon activity and inflammatory responses in severe COVID-19 patients. Science (80-.). (2020) doi:10.1126/science.abc6027.
19. Szabo, P. A. et al. Analysis of respiratory and systemic immune responses in COVID-19 reveals mechanisms of disease pathogenesis. medRxiv (2020) doi:10.1101/2020.10.15.20208041.
20. Speranza, E. et al. Single-cell RNA sequencing reveals SARS-CoV-2 infection dynamics in lungs of African green monkeys. Sci. Transl. Med. (2021) doi:10.1126/scitranslmed.abe8146.
21. Munster, V. et al. Respiratory disease and virus shedding in rhesus macaques inoculated with SARS-CoV-2. bioRxiv (2020) doi:10.1101/2020.03.21.001628.
22. Chandrashekar, A. et al. SARS-CoV-2 infection protects against rechallenge in rhesus macaques. Science (80-.). (2020) doi:10.1.126/science.abc4776.
23. Chan, J. F. W. et al. Simulation of the Clinical and Pathological Manifestations of Coronavirus Disease 2019 (COVID-19) in a Golden Syrian Hamster Model: Implications for Disease Pathogenesis and Transmissibility. Clin. Infect. Dis. (2020) doi:10.1093/cid/ciaa325.
24. Sia, S. F. et al. Pathogenesis and transmission of SARS-CoV-2 in golden hamsters. Nature (2020) doi:10.1038/s41586-020-2342-5.
25. Sun, S. H. et al. A Mouse Model of SARS-CoV-2 Infection and Pathogenesis. Cell HostMicrobe (2020) doi:10.1016/j.chom.2020.05.020.
26. Bao, L. et al. The pathogenicity of SARS-CoV-2 in hACE2 transgenic mice. Nature (2020) doi:10.1038/s41586-020-2312-y.
27. Jiang, R. Di et al. Pathogenesis of SARS-CoV-2 in Transgenic Mice Expressing Human Angiotensin-Converting Enzyme 2. Cell (2020) doi:10.1016/j.cell.2020.05.027.
28. Kim, Y. Il et al. Infection and Rapid Transmission of SARS-CoV-2 in Ferrets. Cell Host Microbe (2020) doi:10.1016/j.chom.2020.03.023.
29. Richard, M. et al. SARS-CoV-2 is transmitted via contact and via the air between ferrets. Nat. Commun. (2020) doi:10.1038/s41467-020-17367-2.
30. Ravindra, N. G. et al. Single-cell longitudinal analysis of SARS-CoV-2 infection in human airway epithelium. bioRxiv (2020) doi:10.1101/2020.05.06.081695.
31. Blanco-Melo, D. et al. SARS-CoV-2 launches a unique transcriptional signature from in vitro, ex vivo, and in vivo systems. bioRxiv (2020) doi:10.1101/2020.03.24.004655.
32. Zhang, Q. et al. Inborn errors of type I IFN immunity in patients with life-threatening COVID-19. Science (80-.). (2020) doi:10.1.126/science.abd4570.
33. Bastard, P. et al. Autoantibodies against type I IFNs in patients with life-threatening COVID-19. Science (80-.). (2020) doi:10.1126/science.abd4585.
34. Combes, A. J. et al. Global Absence and Targeting of Protective Immune States in Severe COVID-19. bioRxiv (2020).
35. Sungnak, W. et al. SARS-CoV-2 entry factors are highly expressed in nasal epithelial cells together with innate immune genes. Nat. Med. (2020) doi:10.1038/s41591-020-0868-6.
36. Ziegler, C. G. K. et al. SARS-CoV-2 Receptor ACE2 Is an Interferon-Stimulated Gene in Human Airway Epithelial Cells and Is Detected in Specific Cell Subsets across Tissues. Cell (2020) doi:10.1016/j.cell.2020.04.035.
37. Huang, N. et al. Integrated Single-Cell Atlases Reveal an Oral SARS-CoV-2 Infection and Transmission Axis. medRxiv (2020).
38. Muus, C. et al. Integrated analyses of single-cell atlases reveal age, gender, and smoking status associations with cell type-specific expression of mediators of SARS-CoV-2 viral entry and highlights inflammatory programs in putative target cells. bioRxiv 2020.04.19.049254 (2020) doi:10.1101/2020.04.19.049254.
39. Lukassen, S. et al. SARS-CoV-2 receptor ACE2 and TMPRSS2 are predominantly expressed in a transient secretory cell type in subsegmental bronchial branches. bioRxiv (2020) doi:10.1101/2020.03.13.991455.
40. Chua, R. L. et al. COVID-19 severity correlates with airway epithelium-immune cell interactions identified by single-cell analysis. Nat. Biotechnol. (2020) doi:10.1038/s41587-020-0602-4.
41. Schaefer, I. M. et al. In situ detection of SARS-CoV-2 in lungs and airways of patients with COVID-19. Mod. Pathol. (2020) doi:10.1038/s41379-020-0595-z.
42. Hou, Y. J. et al. SARS-CoV-2 Reverse Genetics Reveals a Variable Infection Gradient in the Respiratory Tract. Cell (2020) doi:10.1016/j.cell.2020.05.042.
43. Zhu, N. et al. Morphogenesis and cytopathic effect of SARS-CoV-2 infection in human airway epithelial cells. Nat. Commun. (2020) doi:10.1038/s41467-020-17796-z.
44. Gierahn, T. M. et al. Seq-Well: Portable, low-cost rna sequencing of single cells at high throughput. Nat. Methods (2017) doi:10.1038/nmeth.4179.
45. Hughes, T. K. et al. Highly Efficient, Massively-Parallel Single-Cell RNA-Seq Reveals Cellular States and Molecular Features of Human Skin Pathology. bioRxiv (2019) doi:10.1101/689273.
46. Aicher, T. P. et al. Seq-Well: A sample-efficient, portable picowell platform for massively parallel single-cell RNA sequencing. in Methods in Molecular Biology (2019). doi:10.1007/978-1-4939-9240-9_8.
47. Ordovas-Montanes, J. et al. Allergic inflammatory memory in human respiratory epithelial progenitor cells. Nature (2018) doi:10.1038/s41586-018-0449-8.
48. Garcia, S. R. et al. Novel dynamics of human mucociliary differentiation revealed by single-cell RNA sequencing of nasal epithelial cultures. Dev. (2019) doi:10.1242/dev.177428.
49. Montoro, D. T. et al. A revised airway epithelial hierarchy includes CFTR-expressing ionocytes. Nature (2018) doi:10.1038/s41586-018-0393-7.
50. Plasschaert, L. W. et al. A single-cell atlas of the airway epithelium reveals the CFTR-rich pulmonary ionocyte. Nature (2018) doi:10.1038/s41586-018-0394-6.
51. Hoffmann, M. et al. SARS-CoV-2 Cell Entry Depends on ACE2 and TMPRSS2 and Is Blocked by a Clinically Proven Protease Inhibitor. Cell (2020) doi:10.1016/j.cell.2020.02.052.
52. Li, W. et al. Angiotensin-converting enzyme 2 is a functional receptor for the SARS coronavirus. Nature (2003) doi:10.1038/nature02145.
53. Yan, R. et al. Structural basis for the recognition of SARS-CoV-2 by full-length human ACE2. Science (80-.). (2020) doi:10.1.126/science.abb2762.
54. Wrapp, D. et al. Cryo-EM structure of the 2019-nCoV spike in the prefusion conformation. Science (80-.). (2020) doi:10.1126/science.aax0902.
55. Wang, Q. et al. Structural and Functional Basis of SARS-CoV-2 Entry by Using Human ACE2. Cell (2020) doi:10.1016/j.cell.2020.03.045.
56. Daniloski, Z. et al. Identification of Required Host Factors for SARS-CoV-2 Infection in Human Cells. Cell (2021) doi:10.1016/j.cell.2020.10.030.
57. Wang, R. et al. Genetic Screens Identify Host Factors for SARS-CoV-2 and Common Cold Coronaviruses. Cell (2021) doi:10.1016/j.cell.2020.12.004.
58. Wei, J. et al. Genome-wide CRISPR Screens Reveal Host Factors Critical for SARS-CoV-2 Infection. Cell (2021) doi:10.1016/j.cell.2020.10.028.
59. Schneider, W. M. et al. Genome-Scale Identification of SARS-CoV-2 and Pan-coronavirus Host Factor Networks. Cell (2021) doi:10.1016/j.cell.2020.12.006.
60. Broggi, A., Granucci, F. & Zanoni, I. Type III interferons: Balancing tissue tolerance and resistance to pathogen invasion. J. Exp. Med. (2020) doi:10.1084/jem.20190295.

### Tables

TABLE 1
Cell Type Marker Genes (related to FIGS. 1, 2, 9)

| | | | | | |
|---|---|---|---|---|---|
| cluster | gene | cluster | gene | cluster | gene |

Table 1A. Coarse Cell Types (see FIG. 1)

| | | | | | |
|---|---|---|---|---|---|
| Basal Cells | KRT5 | Ciliated Cells | ABCA5 | Goblet Cells | BICDL2 |
| Basal Cells | KRT15 | Ciliated Cells | NUDT14 | Goblet Cells | KRT18 |
| Basal Cells | COL7A1 | Ciliated Cells | TEX26 | Goblet Cells | ALDH1A1 |
| Basal Cells | DST | Ciliated Cells | C2CD3 | Goblet Cells | PGD |
| Basal Cells | EGR1 | Ciliated Cells | DDX3Y | Goblet Cells | GK5 |
| Basal Cells | FOS | Ciliated Cells | ANKRD39 | Goblet Cells | NR2F6 |
| Basal Cells | TP63 | Ciliated Cells | ALKBH5 | Goblet Cells | HLA-B |
| Basal Cells | EGFR | Ciliated Cells | WNT9A | Goblet Cells | RAB37 |
| Basal Cells | FOSB | Ciliated Cells | HSDL2 | Goblet Cells | NFAT5 |
| Basal Cells | EPAS1 | Ciliated Cells | SAT2 | Goblet Cells | PTPN13 |
| Basal Cells | LAMB1 | Ciliated Cells | PPP2CB | Goblet Cells | POR |
| Basal Cells | FGFR3 | Ciliated Cells | MYH10 | Goblet Cells | AKR1A1 |
| Basal Cells | TNC | Ciliated Cells | CDC42BPG | Goblet Cells | TACSTD2 |
| Basal Cells | KRT17 | Ciliated Cells | C3orf52 | Goblet Cells | TSPO |
| Basal Cells | FAT2 | Ciliated Cells | PARP14 | Goblet Cells | DTX4 |
| Basal Cells | JUN | Ciliated Cells | TCF25 | Goblet Cells | MDK |
| Basal Cells | SERPINF1 | Ciliated Cells | PRPF40B | Goblet Cells | SLC26A2 |
| Basal Cells | S100A2 | Ciliated Cells | GSDMD | Goblet Cells | CTSB |
| Basal Cells | POSTN | Ciliated Cells | GOLGA2 | Goblet Cells | ATP13A5 |
| Basal Cells | CA12 | Ciliated Cells | CYP27A1 | Goblet Cells | PADI1 |
| Basal Cells | HSPA1A | Ciliated Cells | OTUD4 | Goblet Cells | HPGD |
| Basal Cells | OBSCN | Ciliated Cells | BTC | Goblet Cells | CTSC |
| Basal Cells | PABPC1 | Ciliated Cells | WDR45B | Goblet Cells | ABLIM1 |
| Basal Cells | FMO2 | Ciliated Cells | SGMS2 | Goblet Cells | SLC12A2 |
| Basal Cells | SEMA5A | Ciliated Cells | CCNDBP1 | Goblet Cells | SORL1 |
| Basal Cells | ADAM28 | Ciliated Cells | LINC01436 | Goblet Cells | RNF152 |
| Basal Cells | RPLP1 | Ciliated Cells | WNK1 | Goblet Cells | CFB |
| Basal Cells | HSPA1B | Ciliated Cells | ATR | Goblet Cells | SEL1L3 |
| Basal Cells | SLC38A2 | Ciliated Cells | WDR77 | Goblet Cells | SLC9A3R1 |
| Basal Cells | CD81 | Ciliated Cells | GLB1L2 | Goblet Cells | SORD |
| Basal Cells | LAMA5 | Ciliated Cells | NPTN | Goblet Cells | TSHZ2 |
| Basal Cells | RPL8 | Ciliated Cells | PITRM1 | Goblet Cells | NCOA4 |
| Basal Cells | MKL2 | Ciliated Cells | CEP104 | Goblet Cells | MGST1 |
| Basal Cells | ALDH3A2 | Ciliated Cells | CEP131 | Goblet Cells | MYOF |
| Basal Cells | KLF4 | Ciliated Cells | TMEM14B | Goblet Cells | FAM107B |
| Basal Cells | RPL3 | Ciliated Cells | GCLM | Goblet Cells | DUOXA2 |
| Basal Cells | TXNIP | Ciliated Cells | TMEM68 | Goblet Cells | PTGES |
| Basal Cells | CD44 | Ciliated Cells | DNAAF2 | Goblet Cells | SLC4A4 |
| Basal Cells | RPL18 | Ciliated Cells | RASEF | Goblet Cells | ATP10B |
| Basal Cells | RASSF6 | Ciliated Cells | LARP1 | Goblet Cells | CYP2B6 |
| Basal Cells | SFN | Ciliated Cells | GABARAPL2 | Goblet Cells | TET2 |
| Basal Cells | ID1 | Ciliated Cells | WEE2-AS1 | Goblet Cells | ADIRF |
| Basal Cells | TNS1 | Ciliated Cells | RALGDS | Goblet Cells | DCXR |
| Basal Cells | IL33 | Ciliated Cells | TMEM59 | Goblet Cells | STK39 |
| Basal Cells | IER2 | Ciliated Cells | UBB | Goblet Cells | PLA2R1 |
| Basal Cells | CAPN13 | Ciliated Cells | CCT5 | Goblet Cells | FUT3 |
| Basal Cells | RPS6 | Ciliated Cells | HRASLS2 | Goblet Cells | PKM |
| Basal Cells | MT1X | Ciliated Cells | CWH43 | Goblet Cells | ALCAM |
| Basal Cells | RPS18 | Ciliated Cells | SETD2 | Goblet Cells | TCIRG1 |
| Basal Cells | TSHZ2 | Ciliated Cells | MLEC | Goblet Cells | GALE |
| Basal Cells | EEF2 | Ciliated Cells | STAM2 | Goblet Cells | TM9SF3 |
| Basal Cells | RPL13 | Ciliated Cells | ERLIN2 | Goblet Cells | GALNT12 |
| Basal Cells | RACK1 | Ciliated Cells | EML6 | Goblet Cells | VILL |
| Basal Cells | RPS8 | Ciliated Cells | RPP38 | Goblet Cells | CCND1 |
| Basal Cells | CLSTN1 | Ciliated Cells | NDUFAF3 | Goblet Cells | TCN1 |
| Basal Cells | HSPB1 | Ciliated Cells | MED24 | Goblet Cells | HLA-DRB5 |
| Basal Cells | PTPRZ1 | Ciliated Cells | CRIP1 | Goblet Cells | PROM2 |
| Basal Cells | RPL10A | Ciliated Cells | HSP90AB1 | Goblet Cells | SGK1 |
| Basal Cells | TPT1 | Ciliated Cells | TNFRSF21 | Goblet Cells | FXYD3 |
| Basal Cells | RPLP0 | Ciliated Cells | SAP18 | Goblet Cells | STK38 |
| Basal Cells | JUNB | Ciliated Cells | SORT1 | Goblet Cells | MAGI3 |
| Basal Cells | LMO4 | Ciliated Cells | NME3 | Goblet Cells | SDC1 |
| Basal Cells | MYOF | Ciliated Cells | CXXC1 | Goblet Cells | ST14 |
| Basal Cells | NOP53 | Ciliated Cells | GFPT1 | Goblet Cells | ASPH |
| Basal Cells | RPL4 | Ciliated Cells | MICAL3 | Goblet Cells | NBEAL1 |
| Basal Cells | FLNA | Ciliated Cells | LRWD1 | Goblet Cells | PDXDC1 |
| Basal Cells | COL4A5 | Ciliated Cells | RHPN2 | Goblet Cells | IFITM2 |
| Basal Cells | RPL13A | Ciliated Cells | VPS28 | Goblet Cells | RPS12 |
| Basal Cells | RPS21 | Ciliated Cells | CRACR2B | Goblet Cells | SLC44A2 |
| Basal Cells | RPS16 | Ciliated Cells | CANX | Goblet Cells | BICDL1 |
| Basal Cells | JAG1 | Ciliated Cells | KCTD1 | Goblet Cells | ALDH3A2 |
| Basal Cells | RPS4X | Ciliated Cells | RHBDD2 | Goblet Cells | RHOC |
| Basal Cells | RPL31 | Ciliated Cells | PNISR | Goblet Cells | MAL2 |
| Basal Cells | RPS12 | Ciliated Cells | PLCB2 | Goblet Cells | TMEM160 |
| Basal Cells | IER3 | Ciliated Cells | PEX6 | Goblet Cells | MX1 |
| Basal Cells | RPL7A | Ciliated Cells | HSPB11 | Goblet Cells | ID1 |
| Basal Cells | PIK3R1 | Ciliated Cells | C2CD2L | Goblet Cells | TSPAN8 |
| Basal Cells | CEBPD | Ciliated Cells | ESYT2 | Goblet Cells | SLC5A8 |
| Basal Cells | SLC25A6 | Ciliated Cells | TMEM245 | Goblet Cells | VCL |
| Basal Cells | BCAM | Ciliated Cells | USH1C | Goblet Cells | RAI14 |
| Basal Cells | ZFP36L1 | Ciliated Cells | CDK20 | Goblet Cells | CHL1 |
| Basal Cells | RPL5 | Ciliated Cells | USP51 | Goblet Cells | PRKAR2B |
| Basal Cells | PTPN13 | Ciliated Cells | TTLL6 | Goblet Cells | TMSB10 |
| Basal Cells | PLCH2 | Ciliated Cells | ATXN7L1 | Goblet Cells | FAM114A1 |
| Basal Cells | JAG2 | Ciliated Cells | ERCC1 | Goblet Cells | OAS1 |
| Basal Cells | TNS4 | Ciliated Cells | MAT1A | Goblet Cells | AP1G2 |
| Basal Cells | RPS23 | Ciliated Cells | TMED10 | Goblet Cells | TSTA3 |
| Basal Cells | PRNP | Ciliated Cells | ISCA2 | Goblet Cells | PNISR |
| Basal Cells | RPS5 | Ciliated Cells | PDE4DIP | Goblet Cells | ASCC2 |
| Basal Cells | LRP1 | Ciliated Cells | PRICKLE2 | Goblet Cells | CD82 |
| Basal Cells | RPS19 | Ciliated Cells | NOL6 | Goblet Cells | SYTL2 |
| Basal Cells | RPL11 | Ciliated Cells | PCDH7 | Goblet Cells | PHF20L1 |
| Basal Cells | RPL12 | Ciliated Cells | SPAG7 | Goblet Cells | H6PD |
| Basal Cells | RPL37A | Ciliated Cells | PRPF6 | Goblet Cells | ASAH1 |
| Basal Cells | PTGFRN | Ciliated Cells | SULT1A1 | Goblet Cells | ELL2 |
| Basal Cells | SULT1E1 | Ciliated Cells | CHD6 | Goblet Cells | TPD52L1 |
| Basal Cells | SULF2 | Ciliated Cells | N6AMT1 | Goblet Cells | CRYBG1 |
| Basal Cells | PLEKHG3 | Ciliated Cells | CYTH1 | Goblet Cells | C19orf33 |
| Basal Cells | MAFB | Ciliated Cells | SCP2 | Goblet Cells | RPLP2 |
| Basal Cells | RPS24 | Ciliated Cells | POR | Goblet Cells | BLVRB |
| Basal Cells | ATP1B3 | Ciliated Cells | ZDHHC11 | Goblet Cells | SGSM2 |
| Basal Cells | BOC | Ciliated Cells | HINT2 | Goblet Cells | ACSL3 |
| Basal Cells | PDGFA | Ciliated Cells | CAPN7 | Goblet Cells | HP1BP3 |
| Basal Cells | RPLP2 | Ciliated Cells | SLC2A10 | Goblet Cells | RAPGEFL1 |
| Basal Cells | PRSS23 | Ciliated Cells | GOLM1 | Goblet Cells | S100A13 |
| Basal Cells | IGSF3 | Ciliated Cells | RASA3 | Goblet Cells | ARFGEF1 |
| Basal Cells | NOTCH1 | Ciliated Cells | ARMH4 | Goblet Cells | PPDPF |
| Basal Cells | HLF | Ciliated Cells | CATSPERD | Goblet Cells | SPINT2 |
| Basal Cells | CAVIN1 | Ciliated Cells | C2orf74 | Goblet Cells | PLEKHS1 |
| Basal Cells | PKP1 | Ciliated Cells | C2orf81 | Goblet Cells | SERINC2 |
| Basal Cells | DSC3 | Ciliated Cells | PSMD1 | Goblet Cells | TTC3 |
| Basal Cells | ANOS1 | Ciliated Cells | SRRM2 | Goblet Cells | KIAA1324 |
| Basal Cells | SESN3 | Ciliated Cells | CACHD1 | Goblet Cells | ESRP2 |
| Basal Cells | FLRT3 | Ciliated Cells | MYO1D | Goblet Cells | ZG16B |
| Basal Cells | BTG1 | Ciliated Cells | ANKRD35 | Goblet Cells | LAMTOR5 |
| Basal Cells | NECTIN1 | Ciliated Cells | NEK4 | Goblet Cells | CAPN5 |
| Basal Cells | SNCA | Ciliated Cells | PAXBP1 | Goblet Cells | PODXL |
| Basal Cells | F3 | Ciliated Cells | CTTN | Goblet Cells | PAQR4 |
| Basal Cells | BCL11A | Ciliated Cells | CCDC160 | Goblet Cells | ICA1 |
| Basal Cells | NRG1 | Ciliated Cells | NDUFAB1 | Goblet Cells | VEGFA |
| Basal Cells | CLCA2 | Ciliated Cells | COG7 | Goblet Cells | PIK3R3 |
| Basal Cells | LGR6 | Ciliated Cells | CPSF1 | Goblet Cells | EIF4G1 |
| Basal Cells | AQP5 | Ciliated Cells | DMXL2 | Goblet Cells | CADPS2 |
| Basal Cells | DKK3 | Ciliated Cells | PBRM1 | Goblet Cells | CA12 |
| Basal Cells | ELN | Ciliated Cells | PARK7 | Goblet Cells | HLA-F |
| Basal Cells | BMP7 | Ciliated Cells | SNX3 | Goblet Cells | SSR4 |
| Basal Cells | MARVELD1 | Ciliated Cells | TMED4 | Goblet Cells | CARMIL1 |
| Basal Cells | SNX31 | Ciliated Cells | ASIC1 | Goblet Cells | LIMK2 |
| Basal Cells | DLK2 | Ciliated Cells | CFAP206 | Goblet Cells | CHD9 |
| Basal Cells | SNAI2 | Ciliated Cells | NARS | Goblet Cells | BACE2 |
| Basal Cells | NGFR | Ciliated Cells | DHX30 | Goblet Cells | RPL37A |
| Ciliated Cells | DNAH5 | Ciliated Cells | HOOK1 | Goblet Cells | QSOX1 |
| Ciliated Cells | SYNE1 | Ciliated Cells | VPS25 | Goblet Cells | KMT2C |
| Ciliated Cells | DNAAF1 | Ciliated Cells | CCL28 | Goblet Cells | TRIP6 |
| Ciliated Cells | CFAP157 | Ciliated Cells | SPR | Goblet Cells | CCNO |
| Ciliated Cells | DLEC1 | Ciliated Cells | ZNF516 | Goblet Cells | SRSF11 |
| Ciliated Cells | DNAH11 | Ciliated Cells | PSAP | Goblet Cells | SREBF1 |
| Ciliated Cells | DNAH3 | Ciliated Cells | C2CD5 | Goblet Cells | ECE1 |
| Ciliated Cells | CAPS | Ciliated Cells | ZFP3 | Goblet Cells | SLC39A7 |
| Ciliated Cells | DNAH12 | Ciliated Cells | STAU1 | Goblet Cells | LDHA |
| Ciliated Cells | CFAP100 | Ciliated Cells | RETREG1 | Goblet Cells | ERBB2 |
| Ciliated Cells | CCDC17 | Ciliated Cells | PLPP2 | Goblet Cells | CXCL1 |
| Ciliated Cells | CDHR3 | Ciliated Cells | SDK1 | Goblet Cells | LPP |
| Ciliated Cells | HYDIN | Ciliated Cells | KIAA1211 | Goblet Cells | IDH1 |
| Ciliated Cells | CFAP46 | Ciliated Cells | RANBP10 | Goblet Cells | TCF25 |
| Ciliated Cells | FHAD1 | Ciliated Cells | NUPR1 | Goblet Cells | RPL27A |
| Ciliated Cells | DNAH9 | Ciliated Cells | SRPX2 | Goblet Cells | GGT6 |
| Ciliated Cells | ERICH3 | Ciliated Cells | SURF1 | Goblet Cells | DTX2 |
| Ciliated Cells | DNAH10 | Ciliated Cells | PRPS1 | Goblet Cells | LAMB2 |
| Ciliated Cells | VWA3A | Ciliated Cells | LRRC36 | Goblet Cells | ADI1 |
| Ciliated Cells | DNAH6 | Ciliated Cells | PIR | Goblet Cells | RAB25 |
| Ciliated Cells | SPEF2 | Ciliated Cells | PDK4 | Goblet Cells | LGI1 |
| Ciliated Cells | TPPP3 | Ciliated Cells | METTL27 | Goblet Cells | SH2D4A |
| Ciliated Cells | CFAP43 | Ciliated Cells | PPM1H | Goblet Cells | HDLBP |
| Ciliated Cells | DNAH7 | Ciliated Cells | ATRX | Goblet Cells | DBNL |
| Ciliated Cells | RSPH1 | Ciliated Cells | P4HA1 | Goblet Cells | CTNND1 |
| Ciliated Cells | CFAP44 | Ciliated Cells | PMM1 | Goblet Cells | MVP |
| Ciliated Cells | RP1 | Ciliated Cells | USP11 | Goblet Cells | VWA1 |
| Ciliated Cells | SPAG17 | Ciliated Cells | CHCHD6 | Goblet Cells | ANXA11 |
| Ciliated Cells | LRRIQ1 | Ciliated Cells | TNFRSF14 | Goblet Cells | RHOV |
| Ciliated Cells | DRC3 | Ciliated Cells | ZNF33A | Goblet Cells | TRPM4 |
| Ciliated Cells | CFAP70 | Ciliated Cells | LONP2 | Goblet Cells | NIPAL2 |
| Ciliated Cells | TBC1D8 | Ciliated Cells | NCBP3 | Goblet Cells | KIF13B |
| Ciliated Cells | CFAP54 | Ciliated Cells | HSPA4 | Goblet Cells | MYO5C |
| Ciliated Cells | ZBBX | Ciliated Cells | HDAC7 | Goblet Cells | HOMER2 |
| Ciliated Cells | BEST4 | Ciliated Cells | CNTNAP3 | Goblet Cells | HSD11B2 |
| Ciliated Cells | WDR60 | Ciliated Cells | FUZ | Goblet Cells | MECOM |
| Ciliated Cells | KIAA2012 | Ciliated Cells | YLPM1 | Goblet Cells | EEF1D |
| Ciliated Cells | RRAD | Ciliated Cells | CAB39 | Goblet Cells | BCL6 |
| Ciliated Cells | TMEM190 | Ciliated Cells | ZMAT1 | Goblet Cells | SMC4 |
| Ciliated Cells | CCDC170 | Ciliated Cells | SMDT1 | Goblet Cells | UPF1 |
| Ciliated Cells | C20orf85 | Ciliated Cells | METRN | Goblet Cells | OGFRL1 |
| Ciliated Cells | ZMYND10 | Ciliated Cells | ATP7B | Goblet Cells | RPL19 |
| Ciliated Cells | CFAP45 | Ciliated Cells | ACTR1B | Goblet Cells | TOP1 |
| Ciliated Cells | FRMPD2 | Ciliated Cells | WRAP53 | Goblet Cells | RSBN1L |
| Ciliated Cells | AC007906.2 | Ciliated Cells | TBC1D32 | Goblet Cells | CSTA |
| Ciliated Cells | CSPP1 | Ciliated Cells | TRIP12 | Goblet Cells | AKAP13 |
| Ciliated Cells | DMD | Ciliated Cells | NSD3 | Goblet Cells | PRRC2C |
| Ciliated Cells | DTHD1 | Ciliated Cells | SLAIN2 | Goblet Cells | UNC13B |
| Ciliated Cells | CROCC2 | Ciliated Cells | FAXDC2 | Goblet Cells | GNAI1 |
| Ciliated Cells | CCDC180 | Ciliated Cells | DCAF6 | Goblet Cells | RAB2A |
| Ciliated Cells | CCDC187 | Ciliated Cells | PTPRA | Goblet Cells | FUT6 |
| Ciliated Cells | CCDC40 | Ciliated Cells | ADD3 | Goblet Cells | SLK |
| Ciliated Cells | CDHR4 | Ciliated Cells | NRBP2 | Goblet Cells | TMEM205 |
| Ciliated Cells | DYNC2H1 | Ciliated Cells | TM9SF2 | Goblet Cells | FKBP2 |
| Ciliated Cells | CCDC114 | Ciliated Cells | MECOM | Goblet Cells | DGKA |
| Ciliated Cells | CCDC146 | Ciliated Cells | VSTM2L | Goblet Cells | NAPRT |
| Ciliated Cells | CROCC | Ciliated Cells | AL033397.1 | Goblet Cells | PPA1 |
| Ciliated Cells | EFHC1 | Ciliated Cells | TTC5 | Goblet Cells | SP100 |
| Ciliated Cells | WDR66 | Ciliated Cells | NDUFS3 | Goblet Cells | PLXNB2 |
| Ciliated Cells | C6orf118 | Ciliated Cells | SPATA7 | Goblet Cells | DAP |
| Ciliated Cells | ABCA13 | Ciliated Cells | ARFIP2 | Goblet Cells | MFSD4A |
| Ciliated Cells | NEK10 | Ciliated Cells | TRPC4AP | Goblet Cells | SFN |
| Ciliated Cells | PRR29 | Ciliated Cells | LXN | Goblet Cells | LAMA5 |
| Ciliated Cells | MAATS1 | Ciliated Cells | SIVA1 | Goblet Cells | OST4 |
| Ciliated Cells | TUBB4B | Ciliated Cells | ZCRB1 | Goblet Cells | LINC01133 |
| Ciliated Cells | UBXN10 | Ciliated Cells | TRIM44 | Goblet Cells | CD63 |
| Ciliated Cells | CES1 | Ciliated Cells | ISCU | Goblet Cells | GPT2 |
| Ciliated Cells | IGFBP7 | Ciliated Cells | BAD | Goblet Cells | FASN |
| Ciliated Cells | ALDH3B1 | Ciliated Cells | UCN3 | Goblet Cells | SERPINB11 |
| Ciliated Cells | SPAG6 | Ciliated Cells | AP002026.1 | Goblet Cells | CD74 |
| Ciliated Cells | DNAI1 | Ciliated Cells | TTLL3 | Goblet Cells | LLGL2 |
| Ciliated Cells | C9orf24 | Ciliated Cells | SMIM19 | Goblet Cells | UAP1 |
| Ciliated Cells | LDLRAD1 | Ciliated Cells | STYXL1 | Goblet Cells | GOLGB1 |
| Ciliated Cells | DCDC1 | Ciliated Cells | FAM104B | Goblet Cells | KCNK5 |
| Ciliated Cells | DZIP1L | Ciliated Cells | C5orf15 | Goblet Cells | PAM |
| Ciliated Cells | RSPH4A | Ciliated Cells | BBS5 | Goblet Cells | SAT1 |
| Ciliated Cells | BAIAP3 | Ciliated Cells | STX18 | Goblet Cells | MACC1 |
| Ciliated Cells | FAM216B | Ciliated Cells | TRAF4 | Goblet Cells | SLC16A3 |
| Ciliated Cells | MAPK15 | Ciliated Cells | UBN1 | Goblet Cells | ALDH2 |
| Ciliated Cells | LRRC23 | Ciliated Cells | CDK9 | Goblet Cells | AGRN |
| Ciliated Cells | DNAH1 | Ciliated Cells | MRPL55 | Goblet Cells | REEP3 |
| Ciliated Cells | MNS1 | Ciliated Cells | CAPRIN1 | Goblet Cells | GNE |
| Ciliated Cells | FANK1 | Ciliated Cells | KCNE3 | Goblet Cells | IFI16 |
| Ciliated Cells | PROM1 | Ciliated Cells | NME7 | Goblet Cells | IFI44L |
| Ciliated Cells | CCDC189 | Ciliated Cells | AKR7A2 | Goblet Cells | TMED3 |
| Ciliated Cells | CFAP57 | Ciliated Cells | VEZF1 | Goblet Cells | HNRNPA2B1 |
| Ciliated Cells | MS4A8 | Ciliated Cells | SESN1 | Goblet Cells | GOLGA3 |
| Ciliated Cells | TUBA1A | Ciliated Cells | GPS1 | Goblet Cells | ARHGEF16 |
| Ciliated Cells | AK9 | Ciliated Cells | TEDC1 | Goblet Cells | ADGRG1 |
| Ciliated Cells | CCDC39 | Ciliated Cells | FUCA1 | Goblet Cells | GNL3 |
| Ciliated Cells | IFT57 | Ciliated Cells | PGM2L1 | Goblet Cells | LDLR |
| Ciliated Cells | NWD1 | Ciliated Cells | LINC01513 | Goblet Cells | ARGLU1 |
| Ciliated Cells | CFAP65 | Ciliated Cells | BTAF1 | Goblet Cells | LGALS8 |
| Ciliated Cells | CES4A | Ciliated Cells | DCAF5 | Goblet Cells | RASSF7 |
| Ciliated Cells | HAGHL | Ciliated Cells | HAGH | Goblet Cells | AUP1 |
| Ciliated Cells | DRC1 | Ciliated Cells | MRFAP1 | Goblet Cells | NDUFS7 |
| Ciliated Cells | VWA3B | Ciliated Cells | LYPD6B | Goblet Cells | VPS13D |
| Ciliated Cells | CYP4B1 | Ciliated Cells | TASP1 | Goblet Cells | PABPC4 |
| Ciliated Cells | IQCG | Ciliated Cells | FZD6 | Goblet Cells | FDPS |
| Ciliated Cells | FAM92B | Ciliated Cells | GABPB1-AS1 | Goblet Cells | PPIB |
| Ciliated Cells | CCDC190 | Ciliated Cells | RGS12 | Goblet Cells | HEBP2 |
| Ciliated Cells | TTC25 | Ciliated Cells | SPART | Goblet Cells | GNPNAT1 |
| Ciliated Cells | CETN2 | Ciliated Cells | TLCD1 | Goblet Cells | SCIN |
| Ciliated Cells | SPATA18 | Ciliated Cells | RNF6 | Goblet Cells | FOXN3 |
| Ciliated Cells | ADGB | Ciliated Cells | AMOT | Goblet Cells | VTCN1 |
| Ciliated Cells | LRRC74B | Ciliated Cells | PAF1 | Goblet Cells | TAPBP |
| Ciliated Cells | AGBL2 | Ciliated Cells | AL035661.1 | Goblet Cells | PRPF38B |
| Ciliated Cells | PIFO | Ciliated Cells | TPM2 | Goblet Cells | CCDC186 |
| Ciliated Cells | C5orf49 | Ciliated Cells | CEP95 | Goblet Cells | RNASET2 |
| Ciliated Cells | CFAP74 | Ciliated Cells | PRKDC | Goblet Cells | GALNT5 |
| Ciliated Cells | DNALI1 | Ciliated Cells | SENP7 | Goblet Cells | RPS3 |
| Ciliated Cells | MUC16 | Ciliated Cells | VPS13B | Goblet Cells | RBM39 |
| Ciliated Cells | PRDX5 | Ciliated Cells | PIGR | Goblet Cells | RPL13 |
| Ciliated Cells | ARMC3 | Ciliated Cells | ZNHIT2 | Goblet Cells | NUMA1 |
| Ciliated Cells | CASC1 | Ciliated Cells | AP3D1 | Goblet Cells | AHNAK |
| Ciliated Cells | CD59 | Ciliated Cells | LUC7L | Goblet Cells | APLP2 |
| Ciliated Cells | RFX3 | Ciliated Cells | RSPH10B | Goblet Cells | RPL30 |
| Ciliated Cells | KIF21A | Ciliated Cells | UBXN4 | Goblet Cells | CLDN7 |
| Ciliated Cells | WDR78 | Ciliated Cells | ZNF214 | Goblet Cells | KIF1C |
| Ciliated Cells | IFT172 | Ciliated Cells | SLC16A5 | Goblet Cells | CKAP4 |
| Ciliated Cells | TEKT1 | Ciliated Cells | LY75 | Goblet Cells | GLRX |
| Ciliated Cells | MAP3K19 | Ciliated Cells | SNX17 | Goblet Cells | TST |
| Ciliated Cells | LRRC46 | Ciliated Cells | LRRC61 | Goblet Cells | APRT |
| Ciliated Cells | CEP126 | Ciliated Cells | RAB34 | Goblet Cells | KDELR2 |
| Ciliated Cells | CC2D2A | Ciliated Cells | FIS1 | Goblet Cells | RPS18 |
| Ciliated Cells | GSTA1 | Ciliated Cells | TUSC2 | Goblet Cells | HS3ST1 |
| Ciliated Cells | TRAF3IP1 | Ciliated Cells | ABCC6 | Goblet Cells | MBOAT2 |
| Ciliated Cells | ODF3B | Ciliated Cells | FYCO1 | Goblet Cells | WDR83OS |
| Ciliated Cells | SLC44A4 | Ciliated Cells | AGL | Goblet Cells | EIF2AK3 |
| Ciliated Cells | WDR49 | Ciliated Cells | SON | Goblet Cells | RPL11 |
| Ciliated Cells | TTC21A | Ciliated Cells | ZMYND12 | Goblet Cells | TUBA1C |
| Ciliated Cells | SNTN | Ciliated Cells | PARP4 | Goblet Cells | RNF213 |
| Ciliated Cells | DNAH2 | Ciliated Cells | CLDN4 | Goblet Cells | RPS21 |
| Ciliated Cells | ANKRD18A | Ciliated Cells | STPG1 | Goblet Cells | NEURL3 |
| Ciliated Cells | PLXNB1 | Ciliated Cells | JKAMP | Goblet Cells | UBXN4 |
| Ciliated Cells | NEK5 | Ciliated Cells | RBM19 | Goblet Cells | B3GNT3 |
| Ciliated Cells | C11orf16 | Ciliated Cells | ENO4 | Goblet Cells | BMP3 |
| Ciliated Cells | P4HTM | Ciliated Cells | LLGL2 | Goblet Cells | OAT |
| Ciliated Cells | CCDC80 | Ciliated Cells | SEC16A | Goblet Cells | MET |
| Ciliated Cells | BBOF1 | Ciliated Cells | COLCA2 | Goblet Cells | SMAGP |
| Ciliated Cells | IK | Ciliated Cells | GMPR | Goblet Cells | GLTP |
| Ciliated Cells | WDR90 | Ciliated Cells | ABCD3 | Goblet Cells | PLPP2 |
| Ciliated Cells | CCDC81 | Ciliated Cells | EFCAB7 | Goblet Cells | RALBP1 |
| Ciliated Cells | CFAP69 | Ciliated Cells | PLA2G16 | Goblet Cells | MUC20 |
| Ciliated Cells | PPOX | Ciliated Cells | JAK2 | Goblet Cells | TBC1D9B |
| Ciliated Cells | BASP1 | Ciliated Cells | AKR1A1 | Goblet Cells | AMN |
| Ciliated Cells | CTGF | Ciliated Cells | SPCS1 | Goblet Cells | SON |
| Ciliated Cells | CFAP53 | Ciliated Cells | OXR1 | Goblet Cells | MAOA |
| Ciliated Cells | CLMN | Ciliated Cells | ZNF688 | Goblet Cells | CSTB |
| Ciliated Cells | ALOX15 | Ciliated Cells | TRPT1 | Goblet Cells | RPS19 |
| Ciliated Cells | NUCB2 | Ciliated Cells | TMEM219 | Goblet Cells | YWHAB |
| Ciliated Cells | CCDC113 | Ciliated Cells | LRP2BP | Goblet Cells | HLA-E |
| Ciliated Cells | DNAJA4 | Ciliated Cells | SOX5 | Goblet Cells | NPDC1 |
| Ciliated Cells | TSPAN1 | Ciliated Cells | PSMD2 | Goblet Cells | CCPG1 |
| Ciliated Cells | MAP1A | Ciliated Cells | ATF7IP2 | Goblet Cells | BIRC6 |
| Ciliated Cells | CCDC30 | Ciliated Cells | AC004990.1 | Goblet Cells | GAA |
| Ciliated Cells | DZIP3 | Ciliated Cells | DUS1L | Goblet Cells | SLC35C1 |
| Ciliated Cells | ALDH3A1 | Ciliated Cells | B4GALNT3 | Goblet Cells | MYH9 |
| Ciliated Cells | ANKUB1 | Ciliated Cells | CAPN10 | Goblet Cells | ADGRF1 |
| Ciliated Cells | CKB | Ciliated Cells | DAPP1 | Goblet Cells | RPL35 |
| Ciliated Cells | TMC5 | Ciliated Cells | CYP4X1 | Goblet Cells | GPI |
| Ciliated Cells | ROPN1L | Ciliated Cells | C8orf34 | Goblet Cells | LMO7 |
| Ciliated Cells | AK7 | Ciliated Cells | PKIB | Goblet Cells | FGFR3 |
| Ciliated Cells | ENKUR | Ciliated Cells | SAFB | Goblet Cells | GOLGA2 |
| Ciliated Cells | CIB1 | Ciliated Cells | PLEC | Goblet Cells | BST2 |
| Ciliated Cells | SPEF1 | Ciliated Cells | MBTPS1 | Goblet Cells | IFITM1 |
| Ciliated Cells | TUBA4B | Ciliated Cells | SDHA | Goblet Cells | CYP4F12 |
| Ciliated Cells | WDR63 | Ciliated Cells | UBL3 | Goblet Cells | DUT |
| Ciliated Cells | STK33 | Ciliated Cells | TNKS | Goblet Cells | RAB3D |
| Ciliated Cells | PCM1 | Ciliated Cells | GGA1 | Goblet Cells | HMGCS2 |
| Ciliated Cells | FAM81B | Ciliated Cells | GPR135 | Goblet Cells | ZNF185 |
| Ciliated Cells | TTLL9 | Ciliated Cells | COPS6 | Goblet Cells | CDK2AP2 |
| Ciliated Cells | ARMC4 | Ciliated Cells | EIF3D | Goblet Cells | TP53I11 |
| Ciliated Cells | NEK11 | Ciliated Cells | ENSA | Goblet Cells | LRP10 |
| Ciliated Cells | FOXJ1 | Ciliated Cells | PRRC2B | Goblet Cells | ATP1B3 |
| Ciliated Cells | STOML3 | Ciliated Cells | TEAD1 | Goblet Cells | FAM173A |
| Ciliated Cells | FAM227A | Ciliated Cells | PARD3 | Goblet Cells | RACK1 |
| Ciliated Cells | EZR | Ciliated Cells | BPHL | Goblet Cells | SLCO4C1 |
| Ciliated Cells | KIAA1211L | Ciliated Cells | STOML2 | Goblet Cells | ARPC5 |
| Ciliated Cells | RSPH3 | Ciliated Cells | PAPOLA | Goblet Cells | STEAP2 |
| Ciliated Cells | EFCAB1 | Ciliated Cells | RBM24 | Goblet Cells | TLE2 |
| Ciliated Cells | CCDC88C | Ciliated Cells | RBM10 | Goblet Cells | VAMP5 |
| Ciliated Cells | AL357093.2 | Ciliated Cells | ADAR | Goblet Cells | TUBB |
| Ciliated Cells | SCO2 | Ciliated Cells | MRPS21 | Goblet Cells | MTDH |
| Ciliated Cells | CCDC173 | Ciliated Cells | LUC7L3 | Goblet Cells | GCNT3 |
| Ciliated Cells | CFAP47 | Ciliated Cells | HSD17B8 | Goblet Cells | MZT2B |
| Ciliated Cells | ANKRD26 | Ciliated Cells | C7orf50 | Goblet Cells | MESP1 |
| Ciliated Cells | CCDC153 | Ciliated Cells | FAIM | Goblet Cells | CSNK1A1 |
| Ciliated Cells | SAXO2 | Ciliated Cells | RBKS | Goblet Cells | IRAK3 |
| Ciliated Cells | AKAP9 | Ciliated Cells | CDH26 | Goblet Cells | RNF207 |
| Ciliated Cells | MAP6 | Ciliated Cells | CCDC186 | Goblet Cells | GMDS |
| Ciliated Cells | MOK | Ciliated Cells | SFMBT1 | Goblet Cells | PDXK |
| Ciliated Cells | CLUAP1 | Ciliated Cells | HSPA4L | Goblet Cells | LDLRAP1 |
| Ciliated Cells | WDR54 | Ciliated Cells | SAPCD1-AS1 | Goblet Cells | TXNIP |
| Ciliated Cells | TSNAXIP1 | Ciliated Cells | GIPC2 | Goblet Cells | CXCL16 |
| Ciliated Cells | KLHL6 | Ciliated Cells | INTS10 | Goblet Cells | GSPT1 |
| Ciliated Cells | CCDC33 | Ciliated Cells | CALCOCO1 | Goblet Cells | C1orf116 |
| Ciliated Cells | CFAP73 | Ciliated Cells | RAET1E | Goblet Cells | TMC6 |
| Ciliated Cells | TMEM231 | Ciliated Cells | STAT6 | Goblet Cells | AC025154.2 |
| Ciliated Cells | SLC25A36 | Ciliated Cells | CYTH2 | Goblet Cells | ILVBL |
| Ciliated Cells | EFHB | Ciliated Cells | C20orf194 | Goblet Cells | PTP4A2 |
| Ciliated Cells | CCDC78 | Ciliated Cells | ARL6 | Goblet Cells | PSME2 |
| Ciliated Cells | FABP6 | Ciliated Cells | FGD5-AS1 | Goblet Cells | HNMT |
| Ciliated Cells | CPLANE1 | Ciliated Cells | SENP6 | Goblet Cells | RPS24 |
| Ciliated Cells | IQUB | Ciliated Cells | LRP8 | Goblet Cells | PPL |
| Ciliated Cells | ECT2L | Ciliated Cells | Z95115.1 | Goblet Cells | CTDSPL |
| Ciliated Cells | CHST9 | Ciliated Cells | LRRC73 | Goblet Cells | PON2 |
| Ciliated Cells | TCTEX1D4 | Ciliated Cells | AP001207.3 | Goblet Cells | CHMP3 |
| Ciliated Cells | MORN2 | Ciliated Cells | PCYT1B | Goblet Cells | MAGT1 |
| Ciliated Cells | IFT140 | Ciliated Cells | ATG9B | Goblet Cells | ARHGDIB |
| Ciliated Cells | CCDC191 | Ciliated Cells | MACROD2 | Goblet Cells | LZTS3 |
| Ciliated Cells | MDH1B | Ciliated Cells | ACCS | Goblet Cells | RPL7A |
| Ciliated Cells | LRRC6 | Ciliated Cells | C1orf141 | Goblet Cells | PPCS |
| Ciliated Cells | KIF27 | Ciliated Cells | ERCC3 | Goblet Cells | TRIM7 |
| Ciliated Cells | ALS2CR12 | Ciliated Cells | STK40 | Goblet Cells | FGFR2 |
| Ciliated Cells | ARHGAP18 | Ciliated Cells | RAB4A | Goblet Cells | PLPP1 |
| Ciliated Cells | CFAP52 | Ciliated Cells | SPRYD3 | Goblet Cells | DNAJC15 |
| Ciliated Cells | C11orf88 | Ciliated Cells | COQ8A | Goblet Cells | RPS6 |
| Ciliated Cells | ATP5IF1 | Ciliated Cells | PSMG3 | Goblet Cells | PABPC1 |
| Ciliated Cells | NUDC | Ciliated Cells | BRD8 | Goblet Cells | EML4 |
| Ciliated Cells | UFC1 | Ciliated Cells | EVA1C | Goblet Cells | LPCAT4 |
| Ciliated Cells | GSTA2 | Ciliated Cells | CD200R1 | Goblet Cells | HSD17B10 |
| Ciliated Cells | CRIP2 | Ciliated Cells | PITPNA | Goblet Cells | RPS9 |
| Ciliated Cells | DUOX1 | Ciliated Cells | DHX57 | Goblet Cells | DDX17 |
| Ciliated Cells | PPP1R16A | Ciliated Cells | AL163051.1 | Goblet Cells | SYTL5 |
| Ciliated Cells | GAS2L2 | Ciliated Cells | CREBZF | Goblet Cells | MTUS1 |
| Ciliated Cells | PRSS12 | Ciliated Cells | UPF2 | Goblet Cells | RPLP1 |
| Ciliated Cells | LRRC10B | Ciliated Cells | ITGA3 | Goblet Cells | RNPEPL1 |
| Ciliated Cells | LRRC71 | Ciliated Cells | MAP3K13 | Goblet Cells | CORO2A |
| Ciliated Cells | C21orf58 | Ciliated Cells | BMS1 | Goblet Cells | TPD52 |
| Ciliated Cells | SYNE2 | Ciliated Cells | XRCC5 | Goblet Cells | AC104126.1 |
| Ciliated Cells | NQ01 | Ciliated Cells | ADH6 | Goblet Cells | CAMK1D |
| Ciliated Cells | ALMS1 | Ciliated Cells | ZNF638 | Goblet Cells | ALG3 |
| Ciliated Cells | ARMC2 | Ciliated Cells | DUSP22 | Goblet Cells | RPS8 |
| Ciliated Cells | SLC7A2 | Ciliated Cells | JTB | Goblet Cells | NDRG2 |
| Ciliated Cells | ATP2C2 | Ciliated Cells | 9-Sep | Goblet Cells | GPRC5C |
| Ciliated Cells | CFAP58 | Ciliated Cells | SETD4 | Goblet Cells | HLA-DRB1 |
| Ciliated Cells | LRP11 | Ciliated Cells | TTC8 | Goblet Cells | KCNS3 |
| Ciliated Cells | ODF2L | Ciliated Cells | RBM25 | Goblet Cells | SYNGR1 |
| Ciliated Cells | IGFBP2 | Ciliated Cells | BCL2L1 | Goblet Cells | SLC16A7 |
| Ciliated Cells | SSBP4 | Ciliated Cells | WBP1L | Goblet Cells | CAVIN2 |
| Ciliated Cells | CCDC65 | Ciliated Cells | HADHA | Goblet Cells | BCL2L15 |
| Ciliated Cells | C9orf116 | Ciliated Cells | SURF2 | Goblet Cells | PRSS8 |
| Ciliated Cells | CAPSL | Ciliated Cells | L3MBTL2 | Goblet Cells | NUCKS1 |
| Ciliated Cells | EFCAB12 | Ciliated Cells | PIN1 | Goblet Cells | DHCR7 |
| Ciliated Cells | SPAG8 | Ciliated Cells | UBN2 | Goblet Cells | CTDSP1 |
| Ciliated Cells | IFT81 | Ciliated Cells | SMC1A | Goblet Cells | NT5C2 |
| Ciliated Cells | SPA17 | Ciliated Cells | WDR73 | Goblet Cells | JUP |
| Ciliated Cells | UCP2 | Ciliated Cells | MFSD2A | Goblet Cells | KNOP1 |
| Ciliated Cells | IQCA1 | Ciliated Cells | MTA1 | Goblet Cells | ATP13A2 |
| Ciliated Cells | CCDC57 | Ciliated Cells | SNRNP70 | Goblet Cells | RPL5 |
| Ciliated Cells | TEKT2 | Ciliated Cells | FAM96B | Goblet Cells | RDH11 |
| Ciliated Cells | ULK4 | Ciliated Cells | TRAF3 | Goblet Cells | RNF181 |
| Ciliated Cells | DNAI2 | Ciliated Cells | PPM1G | Goblet Cells | LAPTM4B |
| Ciliated Cells | C1orf194 | Ciliated Cells | CMPK1 | Goblet Cells | PTMA |
| Ciliated Cells | SPAG16 | Ciliated Cells | TMEM178A | Goblet Cells | RAB27B |
| Ciliated Cells | CFAP221 | Ciliated Cells | OGFR | Goblet Cells | SASH1 |
| Ciliated Cells | RHPN1 | Ciliated Cells | HS3ST6 | Goblet Cells | ALDH3B2 |
| Ciliated Cells | TOGARAM2 | Ciliated Cells | FAM221A | Goblet Cells | SMIM31 |
| Ciliated Cells | SAMD15 | Ciliated Cells | SPIRE2 | Goblet Cells | HM13 |
| Ciliated Cells | GIPR | Ciliated Cells | SUGT1 | Goblet Cells | ZFP36L1 |
| Ciliated Cells | PLEKHG7 | Ciliated Cells | TCTA | Goblet Cells | ERBB3 |
| Ciliated Cells | BAIAP2L1 | Ciliated Cells | ZMAT2 | Goblet Cells | FBLN1 |
| Ciliated Cells | TSGA10 | Ciliated Cells | VWA5A | Goblet Cells | RPS23 |
| Ciliated Cells | PSENEN | Ciliated Cells | NDUFB10 | Goblet Cells | SDCBP2 |
| Ciliated Cells | TNFAIP8L1 | Ciliated Cells | SSUH2 | Goblet Cells | GPRC5B |
| Ciliated Cells | DIAPH2 | Ciliated Cells | LRPAP1 | Goblet Cells | MPST |
| Ciliated Cells | ANKMY1 | Ciliated Cells | ARMC9 | Goblet Cells | RPS2 |
| Ciliated Cells | SLC22A4 | Ciliated Cells | TRADD | Goblet Cells | SPINT1 |
| Ciliated Cells | ANKRD18B | Ciliated Cells | MAN1B1 | Goblet Cells | RPL12 |
| Ciliated Cells | SEC14L3 | Ciliated Cells | ERLEC1 | Goblet Cells | MMP1 |
| Ciliated Cells | C20orf96 | Ciliated Cells | PBXIP1 | Goblet Cells | MYO1C |
| Ciliated Cells | FILIP1 | Ciliated Cells | MRPL41 | Goblet Cells | LRRC8A |
| Ciliated Cells | IFT27 | Ciliated Cells | MYH14 | Goblet Cells | RPL32 |
| Ciliated Cells | C2orf40 | Ciliated Cells | MVP | Goblet Cells | PDZD2 |
| Ciliated Cells | KIAA0556 | Ciliated Cells | BAZ2B | Goblet Cells | KYNU |
| Ciliated Cells | ZNF106 | Ciliated Cells | AGAP3 | Goblet Cells | NACA |
| Ciliated Cells | TTC21B | Ciliated Cells | DOCK1 | Goblet Cells | SEC24D |
| Ciliated Cells | TTC29 | Ciliated Cells | SH3BGRL | Goblet Cells | RPL37 |
| Ciliated Cells | SPAG1 | Ciliated Cells | RING1 | Goblet Cells | RPL23 |
| Ciliated Cells | IQCE | Ciliated Cells | PPP5C | Goblet Cells | PLXDC1 |
| Ciliated Cells | NSUN7 | Ciliated Cells | TAGLN3 | Goblet Cells | ARHGAP1 |
| Ciliated Cells | ENPP5 | Ciliated Cells | SNX2 | Goblet Cells | CYP4B1 |
| Ciliated Cells | TYMP | Ciliated Cells | XPNPEP3 | Goblet Cells | NME1 |
| Ciliated Cells | CEP83 | Ciliated Cells | BCLAF1 | Goblet Cells | CLEC7A |
| Ciliated Cells | FAM183A | Ciliated Cells | SDCCAG8 | Goblet Cells | DYNC1H1 |
| Ciliated Cells | IFT88 | Ciliated Cells | NUB1 | Goblet Cells | HNRNPU |
| Ciliated Cells | EFHC2 | Ciliated Cells | IDNK | Goblet Cells | TSPAN14 |
| Ciliated Cells | CNTRL | Ciliated Cells | PSMA5 | Goblet Cells | RPL14 |
| Ciliated Cells | OMG | Ciliated Cells | DOPEY1 | Goblet Cells | RPS28 |
| Ciliated Cells | ARHGAP39 | Ciliated Cells | ERBB2 | lonocytes | RARRES2 |
| Ciliated Cells | AHNAK2 | Ciliated Cells | CHCHD1 | lonocytes | ADGRF5 |
| Ciliated Cells | TTLL10 | Ciliated Cells | NEDD4L | lonocytes | CFTR |
| Ciliated Cells | SPTBN1 | Ciliated Cells | DDX1 | lonocytes | STAP1 |
| Ciliated Cells | SPPL2B | Ciliated Cells | COPS8 | lonocytes | SCNN1B |
| Ciliated Cells | CGN | Ciliated Cells | ERICH6-AS1 | Ionocytes | CLCNKB |
| Ciliated Cells | UBXN11 | Ciliated Cells | MYO5B | Ionocytes | ITPR2 |
| Ciliated Cells | DYDC2 | Ciliated Cells | SPATA20 | Ionocytes | TFCP2L1 |
| Ciliated Cells | FAM13A | Ciliated Cells | PUM2 | Ionocytes | ASCL3 |
| Ciliated Cells | CEP290 | Ciliated Cells | MATR3 | Ionocytes | ANK2 |
| Ciliated Cells | HMGN3 | Ciliated Cells | KIAA1217 | Ionocytes | RNF152 |
| Ciliated Cells | INTU | Ciliated Cells | MROH1 | Ionocytes | DST |
| Ciliated Cells | STRBP | Ciliated Cells | LDLRAD4 | Ionocytes | THBS1 |
| Ciliated Cells | CDS1 | Ciliated Cells | SLTM | Ionocytes | IGF1 |
| Ciliated Cells | ZNF440 | Ciliated Cells | CRELD2 | Ionocytes | ITIH5 |
| Ciliated Cells | DNPH1 | Ciliated Cells | SSU72 | Ionocytes | ATP6V1A |
| Ciliated Cells | RUVBL1 | Ciliated Cells | CNDP2 | Ionocytes | PPP1R12B |
| Ciliated Cells | TSPAN19 | Ciliated Cells | EFNB3 | Ionocytes | LINC01187 |
| Ciliated Cells | TCTEX1D1 | Ciliated Cells | NEDD8 | Ionocytes | HEPACAM2 |
| Ciliated Cells | TSPAN6 | Ciliated Cells | RTN4 | Ionocytes | SCNN1G |
| Ciliated Cells | GPR162 | Ciliated Cells | PRPF40A | Ionocytes | GOLM1 |
| Ciliated Cells | RUVBL2 | Ciliated Cells | EPS8L2 | Ionocytes | BSND |
| Ciliated Cells | IGFBP5 | Ciliated Cells | BCCIP | Ionocytes | PLCG2 |
| Ciliated Cells | DENND6B | Ciliated Cells | SPATA4 | Ionocytes | DGKI |
| Ciliated Cells | GAS8 | Ciliated Cells | BAIAP2-DT | Ionocytes | SSFA2 |
| Ciliated Cells | CCDC74A | Ciliated Cells | PATL2 | Ionocytes | PDE1C |
| Ciliated Cells | PACRG | Ciliated Cells | STX16 | Ionocytes | FOXI1 |
| Ciliated Cells | SAMHD1 | Ciliated Cells | WDR11 | Ionocytes | APLP2 |
| Ciliated Cells | PLCH1 | Ciliated Cells | RGL1 | Ionocytes | KIT |
| Ciliated Cells | SPATA17 | Ciliated Cells | ZDHHC23 | Ionocytes | LAMA4 |
| Ciliated Cells | IQCD | Ciliated Cells | NAP1L1 | Ionocytes | DMRT2 |
| Ciliated Cells | RIBC1 | Ciliated Cells | CUL1 | Ionocytes | CLNK |
| Ciliated Cells | NPHP1 | Ciliated Cells | MGAT5 | Ionocytes | C1orf115 |
| Ciliated Cells | CD24 | Ciliated Cells | SMARCA4 | Ionocytes | ATP6V1G3 |
| Ciliated Cells | KIF3B | Ciliated Cells | PSMB1 | Ionocytes | TMEM61 |
| Ciliated Cells | METTL7A | Ciliated Cells | EPB41L4A | Ionocytes | CLCNKA |
| Ciliated Cells | PKIG | Ciliated Cells | DYRK2 | Ionocytes | FOXI2 |
| Ciliated Cells | MAP1B | Ciliated Cells | SPIDR | Ionocytes | CEL |
| Ciliated Cells | CEP162 | Ciliated Cells | IFT52 | Ionocytes | PTGER3 |
| Ciliated Cells | SCPEP1 | Ciliated Cells | WDPCP | Ionocytes | SLC14A1 |
| Ciliated Cells | DNAAF4 | Ciliated Cells | PMPCB | Ionocytes | SCUBE2 |
| Ciliated Cells | DYNLL1 | Ciliated Cells | LMBRD1 | Ionocytes | ATP6V0D2 |
| Ciliated Cells | DZIP1 | Ciliated Cells | RUFY1 | Ionocytes | RCAN2 |
| Ciliated Cells | ERGIC3 | Ciliated Cells | ANP32E | Ionocytes | ADCY5 |
| Ciliated Cells | LAMC2 | Ciliated Cells | MCM3AP | Macrophages | CCL3 |
| Ciliated Cells | CABIN1 | Ciliated Cells | CELF1 | Macrophages | VIM |
| Ciliated Cells | CLIC6 | Ciliated Cells | TMEM259 | Macrophages | SRGN |
| Ciliated Cells | TMEM67 | Ciliated Cells | ELF3 | Macrophages | FTL |
| Ciliated Cells | MORN5 | Ciliated Cells | KIAA0895 | Macrophages | LYZ |
| Ciliated Cells | HDGF | Ciliated Cells | EWSR1 | Macrophages | CXCL8 |
| Ciliated Cells | MAPK10 | Ciliated Cells | TEKT3 | Macrophages | TIMP1 |
| Ciliated Cells | C22orf15 | Ciliated Cells | CHD7 | Macrophages | IL1B |
| Ciliated Cells | FMO3 | Ciliated Cells | SCRN1 | Macrophages | TYROBP |
| Ciliated Cells | CLDN3 | Ciliated Cells | AC007405.3 | Macrophages | SPP1 |
| Ciliated Cells | SLC27A2 | Ciliated Cells | MAGEF1 | Macrophages | SOD2 |
| Ciliated Cells | TRMT9B | Ciliated Cells | TOP2B | Macrophages | VCAN |
| Ciliated Cells | TTC16 | Ciliated Cells | RBM38 | Macrophages | PLIN2 |
| Ciliated Cells | C1orf87 | Ciliated Cells | EMC4 | Macrophages | FCER1G |
| Ciliated Cells | CABCOCO1 | Ciliated Cells | ALDH1L1 | Macrophages | SLC11A1 |
| Ciliated Cells | ZNF664 | Ciliated Cells | DCTN3 | Macrophages | PLEK |
| Ciliated Cells | TRAK1 | Ciliated Cells | SNRNP200 | Macrophages | CCL2 |
| Ciliated Cells | DNAJC10 | Ciliated Cells | TGOLN2 | Macrophages | ZEB2 |
| Ciliated Cells | CYB561 | Ciliated Cells | CCDC91 | Macrophages | CTSL |
| Ciliated Cells | DYNLT1 | Ciliated Cells | SGSM2 | Macrophages | CCL3L1 |
| Ciliated Cells | TMEM232 | Ciliated Cells | CD74 | Macrophages | LAPTM5 |
| Ciliated Cells | TTC12 | Ciliated Cells | RAB11FIP4 | Macrophages | BEST1 |
| Ciliated Cells | NFE2L1 | Ciliated Cells | FGF14 | Macrophages | FTH1 |
| Ciliated Cells | ALDH1A1 | Ciliated Cells | AC130456.2 | Macrophages | LGALS1 |
| Ciliated Cells | FAM229B | Ciliated Cells | RHOA | Macrophages | ITGAX |
| Ciliated Cells | MAGED2 | Ciliated Cells | HNRNPM | Macrophages | CD163 |
| Ciliated Cells | RGL2 | Ciliated Cells | ACACA | Macrophages | HSPA6 |
| Ciliated Cells | ADPRHL2 | Ciliated Cells | SMC3 | Macrophages | CD68 |
| Ciliated Cells | MAP9 | Ciliated Cells | TMED1 | Macrophages | CD83 |
| Ciliated Cells | C9orf135 | Ciliated Cells | EPN2 | Macrophages | PLAUR |
| Ciliated Cells | ANK3 | Ciliated Cells | FAM193B | Macrophages | SH3BGRL3 |
| Ciliated Cells | IFT46 | Ciliated Cells | ST6GALNAC1 | Macrophages | LCP1 |
| Ciliated Cells | PLTP | Ciliated Cells | VRK3 | Macrophages | CD44 |
| Ciliated Cells | OSCP1 | Ciliated Cells | CTNNB1 | Macrophages | AIF1 |
| Ciliated Cells | AHI1 | Ciliated Cells | SERP1 | Macrophages | TNFRSF1B |
| Ciliated Cells | TMC4 | Ciliated Cells | ARL6IP4 | Macrophages | NFKBIA |
| Ciliated Cells | CALM1 | Ciliated Cells | PQLC1 | Macrophages | SERPINA1 |
| Ciliated Cells | PPIL6 | Ciliated Cells | BCL2L2 | Macrophages | PSAP |
| Ciliated Cells | GCLC | Ciliated Cells | KIAA0319L | Macrophages | RETN |
| Ciliated Cells | CCDC13 | Ciliated Cells | H2AFJ | Macrophages | BCL2A1 |
| Ciliated Cells | CCDC96 | Ciliated Cells | AC078864.2 | Macrophages | MAFB |
| Ciliated Cells | ENKD1 | Ciliated Cells | IPO11 | Macrophages | ANXA5 |
| Ciliated Cells | SMYD2 | Ciliated Cells | THAP7 | Macrophages | FCN1 |
| Ciliated Cells | KIAA1841 | Ciliated Cells | TCTN3 | Macrophages | ACTB |
| Ciliated Cells | KIF3A | Ciliated Cells | MAP4 | Macrophages | PFN1 |
| Ciliated Cells | CAPN2 | Ciliated Cells | AP1M2 | Macrophages | CTSB |
| Ciliated Cells | UCKL1-AS1 | Ciliated Cells | SRSF11 | Macrophages | OLR1 |
| Ciliated Cells | ZC2HC1A | Ciliated Cells | PNKP | Macrophages | SLC39A8 |
| Ciliated Cells | RSPH9 | Ciliated Cells | ABCB11 | Macrophages | NAMPT |
| Ciliated Cells | WDR19 | Ciliated Cells | FAM81A | Macrophages | CD14 |
| Ciliated Cells | TMEM173 | Ciliated Cells | ROGDI | Macrophages | AQP9 |
| Ciliated Cells | IFT74 | Ciliated Cells | CLDN7 | Macrophages | CYBB |
| Ciliated Cells | SUN1 | Ciliated Cells | NEBL | Macrophages | FLNA |
| Ciliated Cells | TSPAN3 | Ciliated Cells | DNAAF5 | Macrophages | TGFBI |
| Ciliated Cells | AFDN | Ciliated Cells | BICC1 | Macrophages | CCL4L2 |
| Ciliated Cells | ZNF273 | Ciliated Cells | USP10 | Macrophages | AC020656.1 |
| Ciliated Cells | NBEA | Ciliated Cells | C9orf3 | Macrophages | DNAJB1 |
| Ciliated Cells | POFUT2 | Ciliated Cells | LYRM2 | Macrophages | TMSB4X |
| Ciliated Cells | GAPVD1 | Ciliated Cells | HTATSF1 | Macrophages | HSPH1 |
| Ciliated Cells | SRGAP3-AS2 | Ciliated Cells | DIS3L2 | Macrophages | IER3 |
| Ciliated Cells | DNHD1 | Ciliated Cells | HACD3 | Macrophages | SGK1 |
| Ciliated Cells | COQ4 | Ciliated Cells | BIN3 | Macrophages | IER5 |
| Ciliated Cells | DHRS9 | Ciliated Cells | COQ9 | Macrophages | CYBA |
| Ciliated Cells | RFX2 | Ciliated Cells | DROSHA | Macrophages | LILRB2 |
| Ciliated Cells | ANKZF1 | Ciliated Cells | FLOT1 | Macrophages | CLEC5A |
| Ciliated Cells | TCTN1 | Ciliated Cells | CYP4V2 | Macrophages | EMILIN2 |
| Ciliated Cells | COL28A1 | Ciliated Cells | EFTUD2 | Macrophages | CEBPB |
| Ciliated Cells | DMKN | Ciliated Cells | RPAP3 | Macrophages | RASGEF1B |
| Ciliated Cells | SELENBP1 | Ciliated Cells | CMIP | Macrophages | IGSF6 |
| Ciliated Cells | DCDC2B | Ciliated Cells | AKAP11 | Macrophages | HSPA1A |
| Ciliated Cells | LRRC56 | Ciliated Cells | NEAT1 | Macrophages | S100A8 |
| Ciliated Cells | RAB36 | Ciliated Cells | NORAD | Macrophages | ZFAND2A |
| Ciliated Cells | RAD9A | Ciliated Cells | SMARCA5 | Macrophages | ITGB2 |
| Ciliated Cells | CFAP299 | Ciliated Cells | NAA15 | Macrophages | LILRB1 |
| Ciliated Cells | STK36 | Ciliated Cells | DNMBP | Macrophages | GSTO1 |
| Ciliated Cells | RPGRIP1L | Ciliated Cells | TTC39C | Macrophages | PPP1R15A |
| Ciliated Cells | DRC7 | Ciliated Cells | ECHDC2 | Macrophages | CD63 |
| Ciliated Cells | PTPRF | Ciliated Cells | NFX1 | Macrophages | STAB1 |
| Ciliated Cells | C11orf97 | Ciliated Cells | TRAPPC12 | Macrophages | SLAMF7 |
| Ciliated Cells | TTC6 | Ciliated Cells | ENPP3 | Macrophages | ATP2B1 |
| Ciliated Cells | CYB5D1 | Ciliated Cells | CCAR2 | Macrophages | ANPEP |
| Ciliated Cells | AC013264.1 | Ciliated Cells | CKAP5 | Macrophages | C15orf48 |
| Ciliated Cells | CFAP99 | Ciliated Cells | NSMCE1 | Macrophages | HMOX1 |
| Ciliated Cells | JPT2 | Ciliated Cells | TRAPPC8 | Macrophages | KLF10 |
| Ciliated Cells | TNNI3 | Ciliated Cells | PSMD10 | Macrophages | CFL1 |
| Ciliated Cells | CCDC151 | Ciliated Cells | TP53INP1 | Macrophages | ID2 |
| Ciliated Cells | LCA5 | Ciliated Cells | SETX | Macrophages | FPR1 |
| Ciliated Cells | ADH7 | Ciliated Cells | ZBTB4 | Macrophages | CCDC88A |
| Ciliated Cells | UNC119B | Ciliated Cells | HMGN2 | Macrophages | CD74 |
| Ciliated Cells | HIPK1 | Ciliated Cells | UXT | Macrophages | ABCA1 |
| Ciliated Cells | EFCAB6 | Ciliated Cells | CFDP1 | Macrophages | LAIR1 |
| Ciliated Cells | SFXN3 | Ciliated Cells | EIF4G2 | Macrophages | SDCBP |
| Ciliated Cells | WDR34 | Ciliated Cells | OCIAD1 | Macrophages | CTSZ |
| Ciliated Cells | SOD1 | Ciliated Cells | TYK2 | Macrophages | WTAP |
| Ciliated Cells | PLAC8 | Ciliated Cells | ZNF396 | Macrophages | FGR |
| Ciliated Cells | GCC2 | Ciliated Cells | C19orf70 | Macrophages | NPC2 |
| Ciliated Cells | NEK1 | Ciliated Cells | EIF2D | Macrophages | ATP6V1B2 |
| Ciliated Cells | SLFN13 | Ciliated Cells | TMEM175 | Macrophages | NCF2 |
| Ciliated Cells | COL21A1 | Ciliated Cells | EIF5B | Macrophages | PTPRE |
| Ciliated Cells | FAM184A | Ciliated Cells | CCHCR1 | Macrophages | NINJ1 |
| Ciliated Cells | ARMH1 | Ciliated Cells | TRMT11 | Macrophages | MARCKS |
| Ciliated Cells | KNDC1 | Ciliated Cells | L3MBTL1 | Macrophages | CTSS |
| Ciliated Cells | KIF19 | Ciliated Cells | PAPSS1 | Macrophages | SNX10 |
| Ciliated Cells | CCDC24 | Ciliated Cells | DPM3 | Macrophages | CD53 |
| Ciliated Cells | CFAP126 | Ciliated Cells | SNX19 | Macrophages | TPM3 |
| Ciliated Cells | ARL3 | Ciliated Cells | PSIP1 | Macrophages | CD300E |
| Ciliated Cells | KIF9 | Ciliated Cells | DMAP1 | Macrophages | TLR2 |
| Ciliated Cells | PEBP1 | Ciliated Cells | ST6GALNAC2 | Macrophages | CSTB |
| Ciliated Cells | HMGXB3 | Ciliated Cells | BORCS7 | Macrophages | EMP3 |
| Ciliated Cells | FAM174A | Ciliated Cells | AC096637.2 | Macrophages | CCRL2 |
| Ciliated Cells | EFCAB10 | Ciliated Cells | ZNF599 | Macrophages | HIF1A |
| Ciliated Cells | HSD17B13 | Ciliated Cells | SLC30A9 | Macrophages | GRB2 |
| Ciliated Cells | MGLL | Ciliated Cells | NOA1 | Macrophages | CLEC4E |
| Ciliated Cells | EFCAB2 | Ciliated Cells | MEA1 | Macrophages | HSP90AA1 |
| Ciliated Cells | ABHD2 | Ciliated Cells | PABPC1L | Macrophages | SIRPA |
| Ciliated Cells | MPDZ | Ciliated Cells | CNGA4 | Macrophages | LHFPL2 |
| Ciliated Cells | CD164 | Ciliated Cells | COASY | Macrophages | FCGR2A |
| Ciliated Cells | DDX5 | Ciliated Cells | KIAA1191 | Macrophages | C5AR1 |
| Ciliated Cells | ERBB4 | Ciliated Cells | TCEAL3 | Macrophages | TLN1 |
| Ciliated Cells | MYCBPAP | Ciliated Cells | NMRAL1 | Macrophages | GMFG |
| Ciliated Cells | FAM161A | Ciliated Cells | DHX9 | Macrophages | DSE |
| Ciliated Cells | CELSR1 | Ciliated Cells | ZMYM3 | Macrophages | CAPG |
| Ciliated Cells | MTSS1 | Ciliated Cells | PCNT | Macrophages | TYMP |
| Ciliated Cells | CFAP36 | Ciliated Cells | SEC63 | Macrophages | MCEMP1 |
| Ciliated Cells | CDKL1 | Ciliated Cells | NAGA | Macrophages | S100A9 |
| Ciliated Cells | CAPS2 | Ciliated Cells | WDFY3 | Macrophages | SPI1 |
| Ciliated Cells | SPATA6 | Ciliated Cells | SGSM3 | Macrophages | PTPRC |
| Ciliated Cells | UBAC1 | Ciliated Cells | HSF2 | Macrophages | AC243829.4 |
| Ciliated Cells | SELENOW | Ciliated Cells | NDFIP2 | Macrophages | CSF1R |
| Ciliated Cells | TUSC3 | Ciliated Cells | EPHX2 | Macrophages | PLXDC2 |
| Ciliated Cells | UNC93B1 | Ciliated Cells | FBXL2 | Macrophages | LILRB4 |
| Ciliated Cells | SARAF | Ciliated Cells | ZFHX2 | Macrophages | LCP2 |
| Ciliated Cells | ANKRD66 | Ciliated Cells | SIX3-AS1 | Macrophages | HCK |
| Ciliated Cells | KTN1 | Ciliated Cells | BSG | Macrophages | CSGALNACT2 |
| Ciliated Cells | ATXN7L3B | Ciliated Cells | SLC9A3R1 | Macrophages | TREM1 |
| Ciliated Cells | ENAH | Ciliated Cells | WBP1 | Macrophages | PHACTR1 |
| Ciliated Cells | PIH1D2 | Ciliated Cells | LSM4 | Macrophages | OAZ1 |
| Ciliated Cells | UACA | Ciliated Cells | KPNA1 | Macrophages | LST1 |
| Ciliated Cells | SLC25A29 | Ciliated Cells | FAM172A | Macrophages | AL133415.1 |
| Ciliated Cells | PDLIM1 | Ciliated Cells | LMO2 | Macrophages | MNDA |
| Ciliated Cells | WLS | Ciliated Cells | FER | Macrophages | ARPC2 |
| Ciliated Cells | AKAP6 | Ciliated Cells | GNPAT | Macrophages | COTL1 |
| Ciliated Cells | CLBA1 | Ciliated Cells | CRLF1 | Macrophages | PLXNC1 |
| Ciliated Cells | IL5RA | Ciliated Cells | COQ8B | Macrophages | CPVL |
| Ciliated Cells | B3GNT5 | Ciliated Cells | SLC25A10 | Macrophages | CD93 |
| Ciliated Cells | TEX9 | Ciliated Cells | KIAA1522 | Macrophages | LILRB3 |
| Ciliated Cells | GRAMD2A | Ciliated Cells | CEP70 | Macrophages | MS4A7 |
| Ciliated Cells | TMBIM6 | Ciliated Cells | TMEM14A | Macrophages | TFEC |
| Ciliated Cells | STEAP3 | Ciliated Cells | NIT2 | Macrophages | MYO1F |
| Ciliated Cells | CCDC60 | Ciliated Cells | FXR1 | Macrophages | ADGRE2 |
| Ciliated Cells | DNAJB2 | Ciliated Cells | KAZN | Macrophages | ITGA5 |
| Ciliated Cells | ERICH5 | Ciliated Cells | PAIP2 | Macrophages | CCR1 |
| Ciliated Cells | CLU | Ciliated Cells | SMIM14 | Macrophages | MPP1 |
| Ciliated Cells | PLXNB2 | Ciliated Cells | MAN1A2 | Macrophages | HAVCR2 |
| Ciliated Cells | HECTD1 | Ciliated Cells | ZFP90 | Macrophages | SMIM25 |
| Ciliated Cells | C10orf67 | Ciliated Cells | ACSBG1 | Macrophages | CD86 |
| Ciliated Cells | PRKAR1A | Ciliated Cells | CLHC1 | Macrophages | PLA2G7 |
| Ciliated Cells | ANKRD42 | Ciliated Cells | RIPK4 | Macrophages | ITGAM |
| Ciliated Cells | DAW1 | Ciliated Cells | CDCP1 | Macrophages | C3AR1 |
| Ciliated Cells | PLEKHS1 | Ciliated Cells | UBA7 | Macrophages | FCGR1A |
| Ciliated Cells | TP53BP1 | Ciliated Cells | MTFR1L | Mitotic Basal Cells | MKI67 |
| Ciliated Cells | 10-Mar | Ciliated Cells | ORAI2 | Mitotic Basal Cells | TOP2A |
| Ciliated Cells | SPACA9 | Ciliated Cells | SETD6 | Secretory Cells | BPIFA1 |
| Ciliated Cells | TTC26 | Ciliated Cells | RNF40 | Secretory Cells | KRT24 |
| Ciliated Cells | LINC01765 | Ciliated Cells | RAB11A | Secretory Cells | SERPINB3 |
| Ciliated Cells | ANKRD45 | Ciliated Cells | SUPT7L | Secretory Cells | MSMB |
| Ciliated Cells | CCP110 | Ciliated Cells | SPATA33 | Secretory Cells | CXCL1 |
| Ciliated Cells | MUC4 | Ciliated Cells | WWC1 | Secretory Cells | RARRES1 |
| Ciliated Cells | TPPP | Ciliated Cells | RASAL2 | Secretory Cells | CXCL8 |
| Ciliated Cells | CHST6 | Ciliated Cells | HSPA12A | Secretory Cells | KRT4 |
| Ciliated Cells | PTPRN2 | Ciliated Cells | UBTD1 | Secretory Cells | VMO1 |
| Ciliated Cells | IFT122 | Ciliated Cells | TAF7 | Secretory Cells | SERPINB11 |
| Ciliated Cells | IFT22 | Ciliated Cells | PRPF8 | Secretory Cells | S100A2 |
| Ciliated Cells | DIXDC1 | Ciliated Cells | LMAN1 | Secretory Cells | LYPD2 |
| Ciliated Cells | BTBD3 | Ciliated Cells | PREPL | Secretory Cells | KRT7 |
| Ciliated Cells | MYB | Ciliated Cells | NUDT3 | Secretory Cells | BPIFB1 |
| Ciliated Cells | RABL2B | Ciliated Cells | ARGLU1 | Secretory Cells | AQP5 |
| Ciliated Cells | NME5 | Ciliated Cells | KLHDC10 | Secretory Cells | F3 |
| Ciliated Cells | MORN1 | Ciliated Cells | FAM111A | Secretory Cells | CXCL2 |
| Ciliated Cells | DDX17 | Ciliated Cells | CNKSR1 | Secretory Cells | RPL41 |
| Ciliated Cells | TNFRSF19 | Ciliated Cells | KIZ | Secretory Cells | XBP1 |
| Ciliated Cells | PALLD | Ciliated Cells | QARS | Secretory Cells | AGR2 |
| Ciliated Cells | DNAAF3 | Ciliated Cells | AC025181.2 | Secretory Cells | PRSS23 |
| Ciliated Cells | NELL2 | Ciliated Cells | FAM210B | Secretory Cells | NCOA7 |
| Ciliated Cells | C12orf75 | Ciliated Cells | CREBL2 | Secretory Cells | CP |
| Ciliated Cells | DUSP18 | Ciliated Cells | RUFY3 | Secretory Cells | CXCL3 |
| Ciliated Cells | DZANK1 | Ciliated Cells | EIF5 | Secretory Cells | SLC6A14 |
| Ciliated Cells | CBR1 | Ciliated Cells | NUMA1 | Secretory Cells | CXCL17 |
| Ciliated Cells | MLF1 | Ciliated Cells | LIMK2 | Secretory Cells | ASS1 |
| Ciliated Cells | ANXA2 | Ciliated Cells | RRP12 | Secretory Cells | CD55 |
| Ciliated Cells | RPGR | Ciliated Cells | CEP57 | Secretory Cells | KRT19 |
| Ciliated Cells | DPCD | Ciliated Cells | PRUNE2 | Secretory Cells | STEAP4 |
| Ciliated Cells | CEP164 | Ciliated Cells | CTBP1 | Secretory Cells | IGFBP3 |
| Ciliated Cells | NAT14 | Ciliated Cells | NCALD | Secretory Cells | RDH10 |
| Ciliated Cells | PIBF1 | Ciliated Cells | CYHR1 | Secretory Cells | HES1 |
| Ciliated Cells | VNN3 | Ciliated Cells | TFF3 | Secretory Cells | MMP10 |
| Ciliated Cells | CCDC157 | Ciliated Cells | SLC45A4 | Secretory Cells | ADH1C |
| Ciliated Cells | TOGARAM1 | Ciliated Cells | LAS1L | Secretory Cells | TMSB4X |
| Ciliated Cells | CEP128 | Ciliated Cells | RBM43 | Secretory Cells | S100A14 |
| Ciliated Cells | UTRN | Ciliated Cells | POLI | Secretory Cells | MT-ND4L |
| Ciliated Cells | IGSF10 | Ciliated Cells | INTS11 | Secretory Cells | TM4SF1 |
| Ciliated Cells | APOD | Ciliated Cells | CMTM6 | Secretory Cells | C3 |
| Ciliated Cells | SHROOM3 | Ciliated Cells | MRPL43 | Secretory Cells | RPS18 |
| Ciliated Cells | IRX3 | Ciliated Cells | PDPK1 | Secretory Cells | NTS |
| Ciliated Cells | CFAP298 | Ciliated Cells | PNN | Secretory Cells | ANKRD36C |
| Ciliated Cells | DNAL1 | Ciliated Cells | AC010754.1 | Secretory Cells | S100A6 |
| Ciliated Cells | PALMD | Ciliated Cells | PACRGL | Secretory Cells | HSPA5 |
| Ciliated Cells | ZNF487 | Ciliated Cells | CEP41 | Secretory Cells | EPAS1 |
| Ciliated Cells | GSTP1 | Ciliated Cells | TRIM13 | Secretory Cells | WFDC2 |
| Ciliated Cells | NRAV | Ciliated Cells | NDUFA10 | Secretory Cells | MGST1 |
| Ciliated Cells | ATP2A2 | Ciliated Cells | POLR2H | Secretory Cells | PIGR |
| Ciliated Cells | MFSD6 | Ciliated Cells | TMEM138 | Secretory Cells | SERPINB4 |
| Ciliated Cells | POU2AF1 | Ciliated Cells | SNHG10 | Secretory Cells | MUC1 |
| Ciliated Cells | GRIN3B | Ciliated Cells | ORMDL1 | Secretory Cells | RND3 |
| Ciliated Cells | CCDC181 | Ciliated Cells | VPS39 | Secretory Cells | S100P |
| Ciliated Cells | WDR35 | Ciliated Cells | ABI2 | Secretory Cells | ATP1B1 |
| Ciliated Cells | SMPD2 | Ciliated Cells | MMP24OS | Secretory Cells | RPS29 |
| Ciliated Cells | ODF2 | Ciliated Cells | TCEA2 | Secretory Cells | GABRP |
| Ciliated Cells | AKAP14 | Ciliated Cells | PLEKHA7 | Secretory Cells | HSPB1 |
| Ciliated Cells | WDR38 | Ciliated Cells | L3HYPDH | Secretory Cells | SLPI |
| Ciliated Cells | RCAN3 | Ciliated Cells | SVOPL | Secretory Cells | LCN2 |
| Ciliated Cells | ATP9A | Ciliated Cells | RPS6KA1 | Secretory Cells | RPS27 |
| Ciliated Cells | ANKRD37 | Ciliated Cells | SMG7 | Secretory Cells | GPRC5A |
| Ciliated Cells | LRRC43 | Ciliated Cells | AP1G2 | Secretory Cells | RPL34 |
| Ciliated Cells | OSBPL6 | Ciliated Cells | AC087500.1 | Secretory Cells | CYP2F1 |
| Ciliated Cells | ARFGEF3 | Ciliated Cells | ILF3 | Secretory Cells | RPL12 |
| Ciliated Cells | PLCB4 | Ciliated Cells | CRISPLD1 | Secretory Cells | RPS27A |
| Ciliated Cells | ZSCAN18 | Ciliated Cells | ZNF33B | Secretory Cells | UGT2A1 |
| Ciliated Cells | CD164L2 | Ciliated Cells | SMIM26 | Secretory Cells | C19orf33 |
| Ciliated Cells | MYO1E | Ciliated Cells | PHF1 | Secretory Cells | RPL26 |
| Ciliated Cells | KIF6 | Ciliated Cells | MTMR2 | Secretory Cells | RPL35 |
| Ciliated Cells | ENPP4 | Ciliated Cells | ZNF446 | Secretory Cells | CCND1 |
| Ciliated Cells | CCDC69 | Ciliated Cells | EIPR1 | Secretory Cells | LY6E |
| Ciliated Cells | SYTL3 | Ciliated Cells | RIIAD1 | Secretory Cells | KRT8 |
| Ciliated Cells | ZNF474 | Ciliated Cells | AKR1C3 | Secretory Cells | MMACHC |
| Ciliated Cells | APPL2 | Ciliated Cells | TCP1 | Secretory Cells | PI3 |
| Ciliated Cells | CCDC148 | Ciliated Cells | CLIC1 | Secretory Cells | NPM1 |
| Ciliated Cells | STOX1 | Ciliated Cells | AARS | Secretory Cells | ZFP36 |
| Ciliated Cells | MED25 | Ciliated Cells | FMNL2 | Secretory Cells | SAA1 |
| Ciliated Cells | CCDC66 | Ciliated Cells | BOD1L1 | Secretory Cells | RPL39 |
| Ciliated Cells | LZTFL1 | Ciliated Cells | DSCAML1 | Secretory Cells | RPLP0 |
| Ciliated Cells | WFDC6 | Ciliated Cells | NACC2 | Secretory Cells | S100A4 |
| Ciliated Cells | SF3B2 | Ciliated Cells | TMEM50B | Secretory Cells | LDLR |
| Ciliated Cells | DYNLRB2 | Ciliated Cells | DDX46 | Secretory Cells | GLUL |
| Ciliated Cells | TMEM131 | Ciliated Cells | SYS1 | Secretory Cells | RPS6 |
| Ciliated Cells | SYTL2 | Ciliated Cells | SNX7 | Secretory Cells | SOCS3 |
| Ciliated Cells | KCNE1 | Ciliated Cells | TRIP11 | Secretory Cells | MAL2 |
| Ciliated Cells | ZKSCAN1 | Ciliated Cells | TAF1B | Secretory Cells | RPLP1 |
| Ciliated Cells | DALRD3 | Ciliated Cells | SKIV2L | Secretory Cells | YBX1 |
| Ciliated Cells | POLR21 | Ciliated Cells | ELAC2 | Secretory Cells | RPL32 |
| Ciliated Cells | PHIP | Ciliated Cells | ADIPOR1 | Secretory Cells | RPL13A |
| Ciliated Cells | ANKRD65 | Ciliated Cells | RSRP1 | Secretory Cells | COX6A1 |
| Ciliated Cells | SLC23A1 | Ciliated Cells | AQP4-AS1 | Secretory Cells | MSLN |
| Ciliated Cells | TUFM | Ciliated Cells | AHCYL2 | Secretory Cells | KRT18 |
| Ciliated Cells | EXOC3 | Ciliated Cells | SOAT1 | Secretory Cells | RPS24 |
| Ciliated Cells | WDR27 | Ciliated Cells | ARAP1 | Secretory Cells | RPS12 |
| Ciliated Cells | WDR13 | Ciliated Cells | IARS | Secretory Cells | KLF5 |
| Ciliated Cells | POMT2 | Ciliated Cells | ZNHIT1 | Secretory Cells | S100A16 |
| Ciliated Cells | PRDX1 | Ciliated Cells | CFAP97 | Secretory Cells | NFKBIA |
| Ciliated Cells | C12orf74 | Ciliated Cells | ASB1 | Secretory Cells | RPL37A |
| Ciliated Cells | CALM2 | Ciliated Cells | CPNE3 | Secretory Cells | RPL27A |
| Ciliated Cells | RIBC2 | Ciliated Cells | SPON2 | Secretory Cells | B4GALT5 |
| Ciliated Cells | NEIL1 | Ciliated Cells | LMAN2 | Secretory Cells | JUNB |
| Ciliated Cells | COPRS | Ciliated Cells | INTS1 | Secretory Cells | MACC1 |
| Ciliated Cells | MAPRE3 | Ciliated Cells | YWHAQ | Secretory Cells | FGFBP1 |
| Ciliated Cells | EPCAM | Ciliated Cells | MYO6 | Secretory Cells | SLC12A2 |
| Ciliated Cells | TUBGCP2 | Ciliated Cells | ATRAID | Secretory Cells | CALR |
| Ciliated Cells | FSIP1 | Ciliated Cells | TTC30B | Secretory Cells | SGK1 |
| Ciliated Cells | AZIN1 | Ciliated Cells | TCP11L2 | Secretory Cells | RPS28 |
| Ciliated Cells | RGS22 | Ciliated Cells | FGFR1OP | Secretory Cells | CLINT1 |
| Ciliated Cells | 6-Mar | Ciliated Cells | MYO15B | Secretory Cells | ATP5F1E |
| Ciliated Cells | MPC2 | Ciliated Cells | ASL | Secretory Cells | ADAM28 |
| Ciliated Cells | C7orf57 | Ciliated Cells | ATP13A1 | Secretory Cells | HPGD |
| Ciliated Cells | TEKT4 | Ciliated Cells | DRAM2 | Secretory Cells | ST6GAL1 |
| Ciliated Cells | CTNNAL1 | Ciliated Cells | HLTF | Secretory Cells | ALCAM |
| Ciliated Cells | LRBA | Ciliated Cells | CNTLN | Secretory Cells | RPL23 |
| Ciliated Cells | EXOC7 | Ciliated Cells | PARP1 | Secretory Cells | FER1L6 |
| Ciliated Cells | IFT43 | Ciliated Cells | ZRANB2 | Secretory Cells | PSCA |
| Ciliated Cells | SNAPC4 | Ciliated Cells | AP3M2 | Secretory Cells | GAPDH |
| Ciliated Cells | LRRC4 | Ciliated Cells | FIBP | Secretory Cells | TCIM |
| Ciliated Cells | CFLAR | Ciliated Cells | VILL | Secretory Cells | REEP3 |
| Ciliated Cells | CES2 | Ciliated Cells | RBL2 | Secretory Cells | TACSTD2 |
| Ciliated Cells | CTSS | Ciliated Cells | MRPL28 | Secretory Cells | RPL31 |
| Ciliated Cells | SPATA6L | Ciliated Cells | MFSD10 | Secretory Cells | RNF152 |
| Ciliated Cells | INHBB | Ciliated Cells | STAT2 | Secretory Cells | PPA1 |
| Ciliated Cells | AC013470.2 | Ciliated Cells | SOCS4 | Secretory Cells | ANPEP |
| Ciliated Cells | FMN1 | Ciliated Cells | ARF1 | Secretory Cells | RHOV |
| Ciliated Cells | STK11IP | Ciliated Cells | TDRD7 | Secretory Cells | SERF2 |
| Ciliated Cells | PFN2 | Ciliated Cells | POMGNT2 | Secretory Cells | RPL13 |
| Ciliated Cells | MYCBP | Ciliated Cells | CHMP5 | Secretory Cells | ALPL |
| Ciliated Cells | MIPEP | Ciliated Cells | SCRN2 | Secretory Cells | CTSC |
| Ciliated Cells | USP2 | Ciliated Cells | USP21 | Secretory Cells | ALDH1A1 |
| Ciliated Cells | TAX1BP1 | Ciliated Cells | IRF6 | Secretory Cells | PABPC1 |
| Ciliated Cells | TMEM63A | Ciliated Cells | PELI1 | Secretory Cells | CYP2B6 |
| Ciliated Cells | COLCA1 | Ciliated Cells | CXXC4 | Secretory Cells | TMSB10 |
| Ciliated Cells | CUEDC1 | Ciliated Cells | USO1 | Secretory Cells | SAT1 |
| Ciliated Cells | SRI | Ciliated Cells | KDM1A | Secretory Cells | CEACAM6 |
| Ciliated Cells | CFAP300 | Ciliated Cells | INO80E | Secretory Cells | B2M |
| Ciliated Cells | GLB1L | Ciliated Cells | SYPL1 | Secretory Cells | TPT1 |
| Ciliated Cells | GOLGB1 | Ciliated Cells | SLC25A14 | Squamous Cells | SPRR3 |
| Ciliated Cells | PLEKHB1 | Ciliated Cells | ALS2 | Squamous Cells | KRT13 |
| Ciliated Cells | LRTOMT | Ciliated Cells | ACTN1 | Squamous Cells | MAL |
| Ciliated Cells | TJP3 | Ciliated Cells | SF3B5 | Squamous Cells | EMP1 |
| Ciliated Cells | BBS9 | Ciliated Cells | C4orf33 | Squamous Cells | S100A9 |
| Ciliated Cells | CDC14A | Ciliated Cells | PAIP2B | Squamous Cells | CEACAM5 |
| Ciliated Cells | FBXO31 | Ciliated Cells | IPO5 | Squamous Cells | SCEL |
| Ciliated Cells | PASK | Ciliated Cells | MB | Squamous Cells | S100A8 |
| Ciliated Cells | CFAP61 | Ciliated Cells | ITPA | Squamous Cells | SPRR2A |
| Ciliated Cells | HACD4 | Ciliated Cells | AGPAT5 | Squamous Cells | RPTN |
| Ciliated Cells | BBS4 | Ciliated Cells | MOB1B | Squamous Cells | KRT6A |
| Ciliated Cells | IGF1R | Ciliated Cells | NOL7 | Squamous Cells | SPRR1B |
| Ciliated Cells | SNED1 | Ciliated Cells | UBR2 | Squamous Cells | CEACAM6 |
| Ciliated Cells | IGF2BP2 | Ciliated Cells | GALNT3 | Squamous Cells | TMPRSS11E |
| Ciliated Cells | CCDC125 | Ciliated Cells | WBP11 | Squamous Cells | TMPRSS11B |
| Ciliated Cells | AHSA1 | Ciliated Cells | ULK3 | Squamous Cells | HSPB8 |
| Ciliated Cells | CTSH | Ciliated Cells | AGPAT4 | Squamous Cells | C15orf48 |
| Ciliated Cells | TCTN2 | Ciliated Cells | TTLL1 | Squamous Cells | KRT78 |
| Ciliated Cells | CLSTN1 | Ciliated Cells | BRD9 | Squamous Cells | KRT23 |
| Ciliated Cells | CD38 | Ciliated Cells | KMT2B | Squamous Cells | HOPX |
| Ciliated Cells | TP73 | Ciliated Cells | PRMT5 | Squamous Cells | LCN2 |
| Ciliated Cells | CSNK1G2 | Ciliated Cells | LRRIQ3 | Squamous Cells | TMPRSS11D |
| Ciliated Cells | SHANK2 | Ciliated Cells | IL20RA | Squamous Cells | IL1RN |
| Ciliated Cells | TRIM2 | Ciliated Cells | SIX3 | Squamous Cells | FAM129B |
| Ciliated Cells | DGKH | Ciliated Cells | TP53TG1 | Squamous Cells | PPL |
| Ciliated Cells | KIF2A | Ciliated Cells | AGPAT2 | Squamous Cells | SPRR2E |
| Ciliated Cells | CERKL | Ciliated Cells | ZNF462 | Squamous Cells | MXD1 |
| Ciliated Cells | EXOC4 | Ciliated Cells | RMDN3 | Squamous Cells | SPNS2 |
| Ciliated Cells | MTURN | Ciliated Cells | TMEM98 | Squamous Cells | TMPRSS2 |
| Ciliated Cells | LRGUK | Ciliated Cells | UBL5 | Squamous Cells | NCCRP1 |
| Ciliated Cells | LRIG1 | Ciliated Cells | SLF1 | Squamous Cells | PRSS27 |
| Ciliated Cells | TSPOAP1 | Ciliated Cells | PCBP2 | Squamous Cells | ECM1 |
| Ciliated Cells | ANKS1A | Ciliated Cells | MED11 | Squamous Cells | KRT17 |
| Ciliated Cells | CYP2S1 | Ciliated Cells | LIMS1 | Squamous Cells | MUC21 |
| Ciliated Cells | TXNRD1 | Ciliated Cells | MXD4 | Squamous Cells | ITGB8 |
| Ciliated Cells | RABL2A | Ciliated Cells | RHOT2 | Squamous Cells | SPRR2D |
| Ciliated Cells | DDR1 | Ciliated Cells | ANKEF1 | Squamous Cells | RHCG |
| Ciliated Cells | EIF4G3 | Ciliated Cells | DDX56 | Squamous Cells | H1F0 |
| Ciliated Cells | JHY | Ciliated Cells | PIGT | Squamous Cells | EPS8L1 |
| Ciliated Cells | SCGB2A1 | Ciliated Cells | IFT20 | Squamous Cells | CRNN |
| Ciliated Cells | GPX4 | Ciliated Cells | AGAP1 | Squamous Cells | TP53INP2 |
| Ciliated Cells | SERPINB6 | Ciliated Cells | TMTC2 | Squamous Cells | NDRG2 |
| Ciliated Cells | MDM1 | Ciliated Cells | PDCD11 | Squamous Cells | S100P |
| Ciliated Cells | VDAC3 | Ciliated Cells | PPID | Squamous Cells | GPRC5A |
| Ciliated Cells | ANKRD36 | Ciliated Cells | KDM5C | Squamous Cells | UCA1 |
| Ciliated Cells | LRRC27 | Ciliated Cells | SLC6A9 | Squamous Cells | ALDH1A3 |
| Ciliated Cells | GALNS | Ciliated Cells | HSP90B1 | Squamous Cells | FBXO32 |
| Ciliated Cells | DNAJB13 | Ciliated Cells | FGGY | Squamous Cells | HIST1H1C |
| Ciliated Cells | REC8 | Ciliated Cells | ULK2 | Squamous Cells | SPINT1 |
| Ciliated Cells | SPTAN1 | Ciliated Cells | ABHD6 | Squamous Cells | SAT1 |
| Ciliated Cells | HHLA2 | Ciliated Cells | ZMIZ2 | Squamous Cells | CEACAM1 |
| Ciliated Cells | MUC15 | Ciliated Cells | NUDT5 | Squamous Cells | KRT80 |
| Ciliated Cells | VPS35 | Ciliated Cells | CHD1L | Squamous Cells | PRSS8 |
| Ciliated Cells | VPS13C | Ciliated Cells | GNG12 | Squamous Cells | TACSTD2 |
| Ciliated Cells | NME9 | Ciliated Cells | CES3 | Squamous Cells | PLAT |
| Ciliated Cells | PZP | Ciliated Cells | SLC2A12 | Squamous Cells | TPM4 |
| Ciliated Cells | CMTM4 | Ciliated Cells | FIP1L1 | Squamous Cells | SPINK7 |
| Ciliated Cells | C4orf3 | Ciliated Cells | UBR5 | Squamous Cells | CDKN2B |
| Ciliated Cells | STX2 | Ciliated Cells | TM7SF2 | Squamous Cells | DUSP5 |
| Ciliated Cells | GBP6 | Ciliated Cells | ERP29 | Squamous Cells | LMO7 |
| Ciliated Cells | PPP4R3B | Ciliated Cells | AKAP8L | Squamous Cells | HIST1H2AC |
| Ciliated Cells | FSD1L | Ciliated Cells | GPR107 | Squamous Cells | PITX1 |
| Ciliated Cells | C4orf47 | Ciliated Cells | PSEN2 | Squamous Cells | FTH1 |
| Ciliated Cells | EP400 | Ciliated Cells | CSTF3 | Squamous Cells | KLK7 |
| Ciliated Cells | GON7 | Ciliated Cells | NFIX | Squamous Cells | TMBIM1 |
| Ciliated Cells | DPY30 | Ciliated Cells | PTPN21 | Squamous Cells | SPINK5 |
| Ciliated Cells | ANAPC5 | Ciliated Cells | HEATR5B | Squamous Cells | PRSS22 |
| Ciliated Cells | KIAA0753 | Ciliated Cells | OVOL2 | Squamous Cells | GCNT3 |
| Ciliated Cells | MAPK8IP1 | Ciliated Cells | OTUD6B-AS1 | Squamous Cells | A2ML1 |
| Ciliated Cells | LRRC49 | Ciliated Cells | CCT8 | Squamous Cells | RAB11FIP1 |
| Ciliated Cells | PERP | Ciliated Cells | RLIM | Squamous Cells | ANXA11 |
| Ciliated Cells | SOX2 | Ciliated Cells | SPSB2 | Squamous Cells | CEACAM7 |
| Ciliated Cells | SQLE | Ciliated Cells | SMYD3 | Squamous Cells | KRT16 |
| Ciliated Cells | SYNGAP1 | Ciliated Cells | ELOF1 | Squamous Cells | SDCBP2 |
| Ciliated Cells | CAST | Ciliated Cells | DCTN1 | Squamous Cells | S100A4 |
| Ciliated Cells | GRHL2 | Ciliated Cells | IGBP1 | Squamous Cells | TTC9 |
| Ciliated Cells | PPP1R7 | Ciliated Cells | CNOT1 | Squamous Cells | SQSTM1 |
| Ciliated Cells | CIR1 | Ciliated Cells | XRCC1 | Squamous Cells | LAMB3 |
| Ciliated Cells | APOBEC4 | Ciliated Cells | PLXNA2 | Squamous Cells | CAP1 |
| Ciliated Cells | TRIM3 | Ciliated Cells | WDTC1 | Squamous Cells | DUOX2 |
| Ciliated Cells | CYSTM1 | Ciliated Cells | PPME1 | Squamous Cells | LGALS3 |
| Ciliated Cells | SRGAP3 | Ciliated Cells | NT5C | Squamous Cells | CAMK2N1 |
| Ciliated Cells | TMEM123 | Ciliated Cells | WDR55 | Squamous Cells | GALNT1 |
| Ciliated Cells | RSPH14 | Ciliated Cells | S100A13 | Squamous Cells | FAM83A |
| Ciliated Cells | LRRC45 | Ciliated Cells | PILRB | Squamous Cells | NECTIN4 |
| Ciliated Cells | TMEM212 | Ciliated Cells | RRBP1 | Squamous Cells | SLC6A14 |
| Ciliated Cells | MORF4L2 | Ciliated Cells | DHX16 | Squamous Cells | RIOK3 |
| Ciliated Cells | STMND1 | Ciliated Cells | ADRB1 | Squamous Cells | ADGRF1 |
| Ciliated Cells | SECISBP2L | Ciliated Cells | ACTN4 | Squamous Cells | XDH |
| Ciliated Cells | SLC37A1 | Ciliated Cells | NSRP1 | Squamous Cells | ANXA1 |
| Ciliated Cells | SLC12A7 | Ciliated Cells | HLA-B | Squamous Cells | PADI1 |
| Ciliated Cells | C1orf158 | Ciliated Cells | HIST2H2BE | Squamous Cells | SLK |
| Ciliated Cells | NELFE | Ciliated Cells | ATP2A3 | Squamous Cells | ACTG1 |
| Ciliated Cells | FBXW9 | Ciliated Cells | NONO | Squamous Cells | TIMP2 |
| Ciliated Cells | ANKRD54 | Ciliated Cells | RPN1 | Squamous Cells | SERPINB2 |
| Ciliated Cells | ABCC5 | Ciliated Cells | UBE2G2 | Squamous Cells | TINAGL1 |
| Ciliated Cells | EVI5 | Ciliated Cells | TMX4 | Squamous Cells | RDH10 |
| Ciliated Cells | PLEKHA5 | Ciliated Cells | LINC00467 | Squamous Cells | AZGP1 |
| Ciliated Cells | FYB2 | Ciliated Cells | DMTN | Squamous Cells | SRD5A3 |
| Ciliated Cells | SLC22A23 | Ciliated Cells | IQGAP2 | Squamous Cells | C1orf116 |
| Ciliated Cells | ANKRD28 | Ciliated Cells | STAT1 | Squamous Cells | NT5C2 |
| Ciliated Cells | LMNTD1 | Ciliated Cells | TULP3 | Squamous Cells | CSTA |
| Ciliated Cells | CEP112 | Ciliated Cells | FAM192A | Squamous Cells | KLK10 |
| Ciliated Cells | CEP170B | Ciliated Cells | CTNS | Squamous Cells | FUT3 |
| Ciliated Cells | C9orf72 | Ciliated Cells | CD81 | Squamous Cells | KLK6 |
| Ciliated Cells | PDLIM4 | Ciliated Cells | MGST3 | Squamous Cells | TRIP10 |
| Ciliated Cells | CDH1 | Ciliated Cells | CSMD1 | Squamous Cells | MYL12A |
| Ciliated Cells | CARS | Ciliated Cells | OAZ1 | Squamous Cells | CST6 |
| Ciliated Cells | HSPBP1 | Ciliated Cells | ERMARD | Squamous Cells | TM4SF1 |
| Ciliated Cells | PIH1D3 | Ciliated Cells | DNAJA2 | Squamous Cells | SAMD9 |
| Ciliated Cells | ACADVL | Ciliated Cells | PPIL4 | Squamous Cells | ZNF185 |
| Ciliated Cells | ALCAM | Ciliated Cells | IRF3 | Squamous Cells | TMPRSS4 |
| Ciliated Cells | KATNB1 | Ciliated Cells | PPP1R26-AS1 | Squamous Cells | SDC1 |
| Ciliated Cells | WRB | Ciliated Cells | ABCD2 | Squamous Cells | PLIN3 |
| Ciliated Cells | AP2B1 | Ciliated Cells | ZNF24 | Squamous Cells | PSCA |
| Ciliated Cells | SMAP2 | Ciliated Cells | NAA35 | Squamous Cells | GALNT5 |
| Ciliated Cells | EPB41L4B | Ciliated Cells | MTIF2 | Squamous Cells | C6orf132 |
| Ciliated Cells | BAIAP2 | Ciliated Cells | ZNF432 | Squamous Cells | EPHA2 |
| Ciliated Cells | SLC20A2 | Ciliated Cells | NCOR1 | Squamous Cells | PLS3 |
| Ciliated Cells | CAT | Ciliated Cells | USP16 | Squamous Cells | GNE |
| Ciliated Cells | KIAA0232 | Ciliated Cells | DDAH2 | Squamous Cells | KRT19 |
| Ciliated Cells | ACO2 | Ciliated Cells | NUCKS1 | Squamous Cells | CLTB |
| Ciliated Cells | TTLL5 | Ciliated Cells | XRN1 | Squamous Cells | ERO1A |
| Ciliated Cells | APBB1 | Ciliated Cells | APOL1 | Squamous Cells | GAN |
| Ciliated Cells | ENOSF1 | Ciliated Cells | ANKMY2 | Squamous Cells | KRT7 |
| Ciliated Cells | BHLHE41 | Ciliated Cells | MUC20 | Squamous Cells | PIK3IP1 |
| Ciliated Cells | TMF1 | Ciliated Cells | NDUFA2 | Squamous Cells | CCNG2 |
| Ciliated Cells | OXTR | Ciliated Cells | AC060780.1 | Squamous Cells | CDKN1A |
| Ciliated Cells | PROS1 | Ciliated Cells | PUF60 | Squamous Cells | MTUS1 |
| Ciliated Cells | LENG8 | Ciliated Cells | MGMT | Squamous Cells | PHLDA1 |
| Ciliated Cells | KLHDC9 | Ciliated Cells | RNF187 | Squamous Cells | EVPL |
| Ciliated Cells | SRD5A2 | Ciliated Cells | TGS1 | Squamous Cells | STEAP4 |
| Ciliated Cells | EBNA1BP2 | Ciliated Cells | OTUD1 | Squamous Cells | TMPRSS11A |
| Ciliated Cells | UBE2H | Ciliated Cells | NAA40 | Squamous Cells | SPECC1 |
| Ciliated Cells | DUOXA1 | Ciliated Cells | MLXIP | Squamous Cells | YPEL5 |
| Ciliated Cells | COPB2 | Ciliated Cells | NHSL1 | Squamous Cells | MAFF |
| Ciliated Cells | DLG5 | Ciliated Cells | NUP153 | Squamous Cells | CTSB |
| Ciliated Cells | SYAP1 | Ciliated Cells | MKRN1 | Squamous Cells | PDLIM5 |
| Ciliated Cells | C17orf97 | Ciliated Cells | RUNDC1 | Squamous Cells | MFSD4A |
| Ciliated Cells | CLIP1 | Ciliated Cells | TBCB | Squamous Cells | CPA4 |
| Ciliated Cells | EPHX1 | Ciliated Cells | LIG3 | Squamous Cells | PTGES |
| Ciliated Cells | ANKRD11 | Ciliated Cells | KRIT1 | Squamous Cells | METRNL |
| Ciliated Cells | GNAS | Ciliated Cells | SRPK2 | Squamous Cells | PRDM1 |
| Ciliated Cells | IQCK | Ciliated Cells | MICALL2 | Squamous Cells | S100A16 |
| Ciliated Cells | MACF1 | Ciliated Cells | TSPAN15 | Squamous Cells | KRT24 |
| Ciliated Cells | SERPINI2 | Ciliated Cells | CHL1 | Squamous Cells | ITPKC |
| Ciliated Cells | AKNA | Ciliated Cells | TNFRSF25 | Squamous Cells | RND3 |
| Ciliated Cells | PHTF1 | Ciliated Cells | COPA | Squamous Cells | TMSB10 |
| Ciliated Cells | CYB5A | Ciliated Cells | FNDC3A | Squamous Cells | STK24 |
| Ciliated Cells | KIAA0825 | Ciliated Cells | EXOSC10 | Squamous Cells | PPDPF |
| Ciliated Cells | MYEF2 | Ciliated Cells | KHDRBS1 | Squamous Cells | LIPH |
| Ciliated Cells | CDK5RAP2 | Ciliated Cells | MRPL24 | Squamous Cells | SASH1 |
| Ciliated Cells | MAK | Ciliated Cells | NRP2 | Squamous Cells | CALML3 |
| Ciliated Cells | PSD3 | Ciliated Cells | VWA7 | Squamous Cells | CNFN |
| Ciliated Cells | HMGN5 | Ciliated Cells | LARP6 | Squamous Cells | JUP |
| Ciliated Cells | PPP4R1 | Ciliated Cells | AC133794.1 | Squamous Cells | MYO5B |
| Ciliated Cells | SEC62 | Ciliated Cells | TMEM218 | Squamous Cells | MYL6 |
| Ciliated Cells | BCAS1 | Ciliated Cells | SIPA1L1 | Squamous Cells | ATP6V1G1 |
| Ciliated Cells | CCDC138 | Ciliated Cells | GHITM | Squamous Cells | LRP10 |
| Ciliated Cells | INTS3 | Ciliated Cells | KRCC1 | Squamous Cells | MYO6 |
| Ciliated Cells | FDXR | Ciliated Cells | C14orf132 | Squamous Cells | PPP1CB |
| Ciliated Cells | SNTB1 | Ciliated Cells | N4BP2L2 | Squamous Cells | PHACTR2 |
| Ciliated Cells | LCA5L | Ciliated Cells | ZZEF1 | Squamous Cells | DUSP4 |
| Ciliated Cells | HNRNPF | Ciliated Cells | LINC00271 | Squamous Cells | LYPD3 |
| Ciliated Cells | AC125611.4 | Ciliated Cells | NAF1 | Squamous Cells | ABHD5 |
| Ciliated Cells | TXLNB | Ciliated Cells | IQCC | Squamous Cells | ST3GAL4 |
| Ciliated Cells | PPP1R14C | Ciliated Cells | MBD1 | Squamous Cells | CD55 |
| Ciliated Cells | ANXA7 | Ciliated Cells | FAM13C | Squamous Cells | CYSRT1 |
| Ciliated Cells | TMEM120B | Ciliated Cells | HSPA8 | Squamous Cells | PTTG1IP |
| Ciliated Cells | FBXO15 | Ciliated Cells | BBS12 | Squamous Cells | FAM84A |
| Ciliated Cells | ATP12A | Ciliated Cells | POLR2B | Squamous Cells | TMEM106B |
| Ciliated Cells | ARHGEF38 | Ciliated Cells | OMA1 | Squamous Cells | RALA |
| Ciliated Cells | ICA1L | Ciliated Cells | RAD23A | Squamous Cells | SORT1 |
| Ciliated Cells | TTLL7 | Ciliated Cells | MRPS26 | Squamous Cells | KRT15 |
| Ciliated Cells | CDC16 | Ciliated Cells | SPSB1 | Squamous Cells | IVL |
| Ciliated Cells | IQCH | Ciliated Cells | GADD45G | Squamous Cells | PCBP1 |
| Ciliated Cells | IFT80 | Ciliated Cells | SLC18B1 | Squamous Cells | UBC |
| Ciliated Cells | ANKFN1 | Ciliated Cells | TCEAL1 | Squamous Cells | WASL |
| Ciliated Cells | MSI2 | Ciliated Cells | PPP1R26 | Squamous Cells | GRHL1 |
| Ciliated Cells | SYT8 | Ciliated Cells | TMCO3 | Squamous Cells | ELF3 |
| Ciliated Cells | FAM47E | Ciliated Cells | CMTR1 | Squamous Cells | CPEB4 |
| Ciliated Cells | LPAR3 | Ciliated Cells | KDM3B | Squamous Cells | GIPC1 |
| Ciliated Cells | RBM20 | Ciliated Cells | ENTPD6 | Squamous Cells | TMEM127 |
| Ciliated Cells | KPNA3 | Ciliated Cells | IPO4 | Squamous Cells | ZFAND6 |
| Ciliated Cells | AP006284.1 | Ciliated Cells | PFKL | Squamous Cells | FOSL2 |
| Ciliated Cells | SSB | Ciliated Cells | DLL1 | Squamous Cells | SLC12A6 |
| Ciliated Cells | TRAP1 | Ciliated Cells | AZI2 | Squamous Cells | PCDH1 |
| Ciliated Cells | MAOB | Ciliated Cells | BTN2A1 | Squamous Cells | TJP1 |
| Ciliated Cells | KIFAP3 | Ciliated Cells | B3GNTL1 | Squamous Cells | RNASE7 |
| Ciliated Cells | SIGIRR | Ciliated Cells | BRF1 | Squamous Cells | PEA15 |
| Ciliated Cells | PRR15 | Ciliated Cells | FANCF | Squamous Cells | HMGA1 |
| Ciliated Cells | EML1 | Ciliated Cells | GAS7 | Squamous Cells | SNX18 |
| Ciliated Cells | DEFB124 | Ciliated Cells | UFL1 | Squamous Cells | EPS8L2 |
| Ciliated Cells | AMPD3 | Ciliated Cells | WDR5B | Squamous Cells | TPT1 |
| Ciliated Cells | BUD23 | Ciliated Cells | EAPP | Squamous Cells | SMAGP |
| Ciliated Cells | KIAA0319 | Ciliated Cells | AC244090.1 | Squamous Cells | ABCA1 |
| Ciliated Cells | HIPK3 | Ciliated Cells | PIGL | Squamous Cells | RMND5A |
| Ciliated Cells | CASZ1 | Ciliated Cells | PAX7 | Squamous Cells | SLC31A2 |
| Ciliated Cells | LRRC34 | Ciliated Cells | ATXN10 | Squamous Cells | ZFAND5 |
| Ciliated Cells | ST5 | Ciliated Cells | EMC2 | Squamous Cells | PTPN3 |
| Ciliated Cells | PTPRT | Ciliated Cells | KRT18 | Squamous Cells | PRRG4 |
| Ciliated Cells | CIRBP | Ciliated Cells | ZNF621 | Squamous Cells | F3 |
| Ciliated Cells | SYT5 | Ciliated Cells | TSC22D1 | Squamous Cells | NET1 |
| Ciliated Cells | TOMM34 | Ciliated Cells | SDR39U1 | Squamous Cells | TRIM29 |
| Ciliated Cells | SREK1 | Ciliated Cells | HRAS | Squamous Cells | HIST3H2A |
| Ciliated Cells | MID1IP1 | Ciliated Cells | GOLGA1 | Squamous Cells | ERBB3 |
| Ciliated Cells | TRAPPC9 | Ciliated Cells | COG4 | Squamous Cells | TUFT1 |
| Ciliated Cells | GNA14 | Ciliated Cells | LINC01315 | Squamous Cells | FCHO2 |
| Ciliated Cells | B9D2 | Ciliated Cells | OAS2 | Squamous Cells | VSIG2 |
| Ciliated Cells | STUB1 | Ciliated Cells | AL035701.1 | Squamous Cells | CALB1 |
| Ciliated Cells | PEAK1 | Ciliated Cells | GTF3C3 | Squamous Cells | KLF5 |
| Ciliated Cells | AC084033.3 | Ciliated Cells | TRIT1 | Squamous Cells | YPEL3 |
| Ciliated Cells | RASSF7 | Ciliated Cells | KDM7A | Squamous Cells | VPS4B |
| Ciliated Cells | ADGRV1 | Ciliated Cells | GLOD4 | Squamous Cells | ABLIM3 |
| Ciliated Cells | PFDN6 | Ciliated Cells | DAZAP2 | Squamous Cells | QSOX1 |
| Ciliated Cells | SPTLC2 | Ciliated Cells | MRFAP1L1 | Squamous Cells | CDKN1B |
| Ciliated Cells | LINC02345 | Ciliated Cells | SGCB | Squamous Cells | USP6NL |
| Ciliated Cells | GRAMD1C | Ciliated Cells | ATP2C1 | Squamous Cells | HEBP2 |
| Ciliated Cells | ANKRD36B | Ciliated Cells | HIGDZA | Squamous Cells | ADIRF |
| Ciliated Cells | DSTN | Ciliated Cells | ZNF853 | Squamous Cells | BAG1 |
| Ciliated Cells | C21orf2 | Ciliated Cells | GTF3C5 | Squamous Cells | RNF11 |
| Ciliated Cells | MYO5C | Ciliated Cells | R3HCC1 | Squamous Cells | ATP6V0E1 |
| Ciliated Cells | SCCPDH | Ciliated Cells | PLA2G10 | Squamous Cells | NABP1 |
| Ciliated Cells | TPGS2 | Ciliated Cells | MDH2 | Squamous Cells | FAM214A |
| Ciliated Cells | EPB41L1 | Ciliated Cells | MIB2 | Squamous Cells | PICALM |
| Ciliated Cells | RBM6 | Ciliated Cells | LARP4B | Squamous Cells | SGTA |
| Ciliated Cells | LMLN | Ciliated Cells | SMARCAD1 | Squamous Cells | CDYL |
| Ciliated Cells | UPF3B | Ciliated Cells | PGK1 | Squamous Cells | UBAP1 |
| Ciliated Cells | CTNNA1 | Ciliated Cells | MRPS9 | Squamous Cells | SLCO4A1 |
| Ciliated Cells | UBA1 | Ciliated Cells | PSMD4 | Squamous Cells | SH3BGRL2 |
| Ciliated Cells | TRIP13 | Ciliated Cells | DNTTIP1 | Squamous Cells | FUT6 |
| Ciliated Cells | GNA11 | Ciliated Cells | TBCA | Squamous Cells | RANBP9 |
| Ciliated Cells | ELN-AS1 | Ciliated Cells | DICER1-AS1 | Squamous Cells | CRYBG2 |
| Ciliated Cells | AHNAK | Ciliated Cells | TMEM179B | Squamous Cells | DOCK9 |
| Ciliated Cells | ESPN | Ciliated Cells | POLR2E | Squamous Cells | DIAPH1 |
| Ciliated Cells | ZDHHC11B | Ciliated Cells | TNRC6B | Squamous Cells | GABARAPL2 |
| Ciliated Cells | DDX42 | Ciliated Cells | CETN3 | Squamous Cells | TACC1 |
| Ciliated Cells | PKD1 | Ciliated Cells | THADA | Squamous Cells | TMEM40 |
| Ciliated Cells | PSMB5 | Ciliated Cells | PCMTD2 | Squamous Cells | IL36RN |
| Ciliated Cells | KDM1B | Ciliated Cells | RABEP1 | Squamous Cells | C2orf54 |
| Ciliated Cells | PIAS3 | Ciliated Cells | CTBS | Squamous Cells | VSIR |
| Ciliated Cells | SPATS2L | Ciliated Cells | RXRA | Squamous Cells | MIR4435-2HG |
| Ciliated Cells | B9D1 | Ciliated Cells | AAED1 | Squamous Cells | PARD6B |
| Ciliated Cells | CUTA | Ciliated Cells | ZFR | Squamous Cells | MBOAT2 |
| Ciliated Cells | SLC25A4 | Ciliated Cells | SELENOO | Squamous Cells | ADAM9 |
| Ciliated Cells | FTO | Ciliated Cells | ERC1 | Squamous Cells | EHD3 |
| Ciliated Cells | HDLBP | Ciliated Cells | CCDC130 | Squamous Cells | BCAT1 |
| Ciliated Cells | EXPH5 | Ciliated Cells | CORO7 | Squamous Cells | HLA-E |
| Ciliated Cells | CCDC74B | Ciliated Cells | TXLNA | Squamous Cells | MUC1 |
| Ciliated Cells | MSH3 | Ciliated Cells | 2-Sep | Squamous Cells | S100A11 |
| Ciliated Cells | TSTD1 | Ciliated Cells | TRERF1 | Squamous Cells | POLB |
| Ciliated Cells | CAMSAP1 | Ciliated Cells | KIAA0100 | Squamous Cells | PDZK1IP1 |
| Ciliated Cells | ABHD12B | Ciliated Cells | COQ10A | Squamous Cells | CHMP1B |
| Ciliated Cells | MST1 | Ciliated Cells | OCEL1 | Squamous Cells | HECA |
| Ciliated Cells | SPATA13 | Ciliated Cells | CNOT10 | Squamous Cells | OXSR1 |
| Ciliated Cells | BCAS3 | Ciliated Cells | CCDC121 | Squamous Cells | GDPD3 |
| Ciliated Cells | RNPC3 | Ciliated Cells | XRRA1 | Squamous Cells | ADIPOR1 |
| Ciliated Cells | MEAF6 | Ciliated Cells | STK16 | Squamous Cells | RNF39 |
| Ciliated Cells | RALB | Ciliated Cells | HTATIP2 | Squamous Cells | CHP1 |
| Ciliated Cells | ARHGAP32 | Ciliated Cells | USP9X | Squamous Cells | CLDN4 |
| Ciliated Cells | PSMB4 | Ciliated Cells | PSMB8 | Squamous Cells | HIST1H2BD |
| Ciliated Cells | ZBED5-AS1 | Ciliated Cells | MARVELD2 | Squamous Cells | PTK6 |
| Ciliated Cells | RNF20 | Ciliated Cells | COPG1 | Squamous Cells | WFDC2 |
| Ciliated Cells | OSBPL3 | Ciliated Cells | UBXN6 | Squamous Cells | LMTK2 |
| Ciliated Cells | CEP89 | Ciliated Cells | BTBD1 | Squamous Cells | PINK1 |
| Ciliated Cells | GPATCH2 | Ciliated Cells | SRSF10 | Squamous Cells | PERP |
| Ciliated Cells | MORN3 | Ciliated Cells | MICU2 | Squamous Cells | ARL8B |
| Ciliated Cells | MAP4K4 | Ciliated Cells | SLIT2 | Squamous Cells | ERV3-1 |
| Ciliated Cells | TPRG1L | Ciliated Cells | EYA1 | Squamous Cells | FOXC1 |
| Ciliated Cells | AC234582.1 | Ciliated Cells | FAM227B | Squamous Cells | NAPRT |
| Ciliated Cells | SFI1 | Ciliated Cells | TOMM7 | Squamous Cells | SPSB3 |
| Ciliated Cells | FOXA1 | Ciliated Cells | FARP2 | Squamous Cells | NDFIP2 |
| Ciliated Cells | C16orf71 | Ciliated Cells | CCDC136 | Squamous Cells | PMAIP1 |
| Ciliated Cells | SMPD3 | Ciliated Cells | IRAK1BP1 | Squamous Cells | RNF10 |
| Ciliated Cells | GBP3 | Ciliated Cells | PMS1 | Squamous Cells | SLPI |
| Ciliated Cells | PITPNM1 | Ciliated Cells | MRPS25 | Squamous Cells | ZNRF1 |
| Ciliated Cells | RYR3 | Ciliated Cells | IRF5 | Squamous Cells | PROM2 |
| Ciliated Cells | ABHD11 | Ciliated Cells | VIM-AS1 | Squamous Cells | TOM1 |
| Ciliated Cells | DNAL4 | Ciliated Cells | LRRCC1 | Squamous Cells | NPC1 |
| Ciliated Cells | ATP2B4 | Ciliated Cells | MTIF3 | Squamous Cells | ARF6 |
| Ciliated Cells | SFSWAP | Ciliated Cells | MAML2 | Squamous Cells | YOD1 |
| Ciliated Cells | IFI27 | Ciliated Cells | TTC30A | Squamous Cells | S100A14 |
| Ciliated Cells | GFM2 | Ciliated Cells | STOX2 | Squamous Cells | PTPRH |
| Ciliated Cells | KATNAL2 | Ciliated Cells | PIP5K1B | Squamous Cells | GABARAPL1 |
| Ciliated Cells | SRSF5 | Ciliated Cells | PYCARD | Squamous Cells | NAMPT |
| Ciliated Cells | P4HA2 | Ciliated Cells | FBXO16 | Squamous Cells | BCL2L11 |
| Ciliated Cells | PFKP | Ciliated Cells | OLFML2A | Squamous Cells | CTSA |
| Ciliated Cells | MZF1-AS1 | Ciliated Cells | PDAP1 | Squamous Cells | EIF4EBP2 |
| Ciliated Cells | ARL13B | Ciliated Cells | GSE1 | Squamous Cells | HES4 |
| Ciliated Cells | YBX3 | Ciliated Cells | PRPSAP1 | Squamous Cells | DUSP10 |
| Ciliated Cells | ACSS1 | Ciliated Cells | ANAPC15 | Squamous Cells | TRIM8 |
| Ciliated Cells | SLC6A6 | Ciliated Cells | KLHL32 | Squamous Cells | KIF1C |
| Ciliated Cells | GTF2F1 | Ciliated Cells | LGALS3BP | Squamous Cells | TMOD3 |
| Ciliated Cells | MRPS31 | Ciliated Cells | ZNF428 | Squamous Cells | AD000090.1 |
| Ciliated Cells | POLQ | Ciliated Cells | ATAD3A | Squamous Cells | TNIP1 |
| Ciliated Cells | TCTE1 | Ciliated Cells | MYCT1 | Squamous Cells | LAD1 |
| Ciliated Cells | DYNLRB1 | Ciliated Cells | YIF1A | Squamous Cells | HBP1 |
| Ciliated Cells | PTK2 | Ciliated Cells | PINLYP | Squamous Cells | CARHSP1 |
| Ciliated Cells | CFAP161 | Ciliated Cells | NGRN | Squamous Cells | ADIPOR2 |
| Ciliated Cells | F11R | Ciliated Cells | EPDR1 | Squamous Cells | PHLDA3 |
| Ciliated Cells | CCDC34 | Ciliated Cells | NSMCE4A | Squamous Cells | TMEM159 |
| Ciliated Cells | PPP1R36 | Ciliated Cells | CD46 | Squamous Cells | SPRR1A |
| Ciliated Cells | DCBLD2 | Ciliated Cells | SNW1 | Squamous Cells | ATG9B |
| Ciliated Cells | SEC14L1 | Ciliated Cells | TMEM14C | Squamous Cells | SLC44A2 |
| Ciliated Cells | PDCD6IP | Ciliated Cells | PARG | Squamous Cells | ATP10B |
| Ciliated Cells | EPPIN | Ciliated Cells | TRIOBP | Squamous Cells | RAB9A |
| Ciliated Cells | BICDL2 | Ciliated Cells | ARVCF | Squamous Cells | PLAU |
| Ciliated Cells | TMEM107 | Ciliated Cells | CSTF2T | Squamous Cells | GRN |
| Ciliated Cells | SATB1 | Ciliated Cells | PDE8B | Squamous Cells | AC019349.1 |
| Ciliated Cells | CASC2 | Ciliated Cells | NUDT12 | Squamous Cells | KYNU |
| Ciliated Cells | C2orf73 | Ciliated Cells | DPAGT1 | Squamous Cells | ATP6V1E1 |
| Ciliated Cells | SPICE1 | Ciliated Cells | PANX2 | Squamous Cells | TGFA |
| Ciliated Cells | TMEM45B | Ciliated Cells | COX8A | Squamous Cells | SPTSSA |
| Ciliated Cells | MINDY4 | Ciliated Cells | OSBP | Squamous Cells | FHL2 |
| Ciliated Cells | MAPK8IP3 | Ciliated Cells | RORC | Squamous Cells | PIP |
| Ciliated Cells | ATP6AP1 | Ciliated Cells | COMMD3 | Squamous Cells | HLA-C |
| Ciliated Cells | PPP1R32 | Ciliated Cells | PARP3 | Squamous Cells | GNA15 |
| Ciliated Cells | SNRNP25 | Ciliated Cells | MRRF | Squamous Cells | SBDS |
| Ciliated Cells | DHX40 | Ciliated Cells | SCAND1 | Squamous Cells | CRB3 |
| Ciliated Cells | PARVA | Ciliated Cells | RNH1 | Squamous Cells | TTC22 |
| Ciliated Cells | PTOV1 | Ciliated Cells | TTLL4 | Squamous Cells | UBE2G1 |
| Ciliated Cells | IDS | Ciliated Cells | MRPL20 | Squamous Cells | USP53 |
| Ciliated Cells | MTSS1L | Ciliated Cells | MEIS2 | Squamous Cells | B3GNT3 |
| Ciliated Cells | AGTRAP | Ciliated Cells | ZNF507 | Squamous Cells | SPIRE1 |
| Ciliated Cells | C11orf49 | Ciliated Cells | MBOAT7 | Squamous Cells | AIF1L |
| Ciliated Cells | FAM166B | Ciliated Cells | REX1BD | Squamous Cells | ITPRIP |
| Ciliated Cells | TRPV4 | Ciliated Cells | CNNM4 | Squamous Cells | UPP1 |
| Ciliated Cells | IFIT1 | Ciliated Cells | GUK1 | Squamous Cells | CTSV |
| Ciliated Cells | AC004832.1 | Ciliated Cells | MTX2 | Squamous Cells | UBE2B |
| Ciliated Cells | RHOU | Ciliated Cells | YIPF6 | Squamous Cells | MAP1LC3B |
| Ciliated Cells | ZC3H6 | Ciliated Cells | MPC1 | Squamous Cells | B3GALT5 |
| Ciliated Cells | CTXN1 | Ciliated Cells | PRKCE | Squamous Cells | HIVEP2 |
| Ciliated Cells | DAPK1 | Ciliated Cells | TIMM23 | Squamous Cells | SDR16C5 |
| Ciliated Cells | PCSK5 | Ciliated Cells | CMBL | Squamous Cells | GPR87 |
| Ciliated Cells | NHLRC4 | Ciliated Cells | SNX14 | Squamous Cells | MPRIP |
| Ciliated Cells | DPP7 | Ciliated Cells | ATG4A | Squamous Cells | HIST1H4H |
| Ciliated Cells | RAB3B | Ciliated Cells | GEMIN8 | Squamous Cells | KAZN |
| Ciliated Cells | NIN | Ciliated Cells | CLDN16 | Squamous Cells | MAP7D1 |
| Ciliated Cells | AL590004.4 | Ciliated Cells | PLA1A | Squamous Cells | C15orf62 |
| Ciliated Cells | PGRMC1 | Ciliated Cells | EIF2B5 | Squamous Cells | GRB7 |
| Ciliated Cells | PRRT3 | Ciliated Cells | DYNC1LI2 | Squamous Cells | TMEM54 |
| Ciliated Cells | CDK10 | Ciliated Cells | TRIM32 | Squamous Cells | MPZL3 |
| Ciliated Cells | WHRN | Ciliated Cells | SNAPC5 | Squamous Cells | VWA1 |
| Ciliated Cells | ATP5F1B | Ciliated Cells | TFCP2 | Squamous Cells | SNX9 |
| Ciliated Cells | REPIN1 | Ciliated Cells | SHQ1 | Squamous Cells | PIM1 |
| Ciliated Cells | GMPPB | Ciliated Cells | CRY1 | Squamous Cells | NECTIN2 |
| Ciliated Cells | SYTL1 | Ciliated Cells | DYRK1B | Squamous Cells | UBE2D1 |
| Ciliated Cells | HECTD4 | Ciliated Cells | ST3GAL6 | Squamous Cells | OVOL1 |
| Ciliated Cells | FER1L5 | Ciliated Cells | ATPAF1 | Squamous Cells | SPHK1 |
| Ciliated Cells | CYB561A3 | Ciliated Cells | XPC | Squamous Cells | SH3GL1 |
| Ciliated Cells | CCNL2 | Ciliated Cells | AGBL5 | Squamous Cells | KLHL24 |
| Ciliated Cells | CCT2 | Ciliated Cells | CMPK2 | Squamous Cells | GPAT3 |
| Ciliated Cells | PDCD6 | Ciliated Cells | DHX38 | Squamous Cells | TINCR |
| Ciliated Cells | GTF2I | Ciliated Cells | SLC35A3 | Squamous Cells | RNF223 |
| Ciliated Cells | C10orf95 | Ciliated Cells | SIRT5 | Squamous Cells | GADD45B |
| Ciliated Cells | DSP | Ciliated Cells | PHLPP1 | Squamous Cells | ATP6V1B2 |
| Ciliated Cells | EBLN3P | Ciliated Cells | DRAIC | Squamous Cells | VSIG10L |
| Ciliated Cells | GLIS3 | Ciliated Cells | SART3 | Squamous Cells | LRRC8A |
| Ciliated Cells | MX1 | Ciliated Cells | MVB12B | Squamous Cells | KIFC3 |
| Ciliated Cells | ACADM | Ciliated Cells | ERICH2 | Squamous Cells | SMIM5 |
| Ciliated Cells | SMIM5 | Ciliated Cells | PCBD1 | Squamous Cells | OSER1 |
| Ciliated Cells | SORBS2 | Ciliated Cells | SEC23B | Squamous Cells | TOR1AIP2 |
| Ciliated Cells | MLPH | Ciliated Cells | KBTBD4 | Squamous Cells | MCL1 |
| Ciliated Cells | ETV6 | Ciliated Cells | FAM107B | Squamous Cells | CDC42SE1 |
| Ciliated Cells | FAM149A | Ciliated Cells | ARRDC4 | Squamous Cells | ATF3 |
| Ciliated Cells | CAPNS1 | Ciliated Cells | UBA5 | Squamous Cells | RRAGC |
| Ciliated Cells | GLT8D1 | Ciliated Cells | B2M | Squamous Cells | TRIM16 |
| Ciliated Cells | CCDC82 | Ciliated Cells | TDRD3 | Squamous Cells | RASSF5 |
| Ciliated Cells | FBXL13 | Ciliated Cells | SMAD3 | Squamous Cells | RNPEPL1 |
| Ciliated Cells | DTX3 | Ciliated Cells | RTP4 | Squamous Cells | SECTM1 |
| Ciliated Cells | PCYT2 | Ciliated Cells | FOXP2 | Squamous Cells | DIRC2 |
| Ciliated Cells | CHD4 | Ciliated Cells | BCL2L13 | Squamous Cells | HSPB1 |
| Ciliated Cells | MKS1 | Ciliated Cells | MRPL18 | Squamous Cells | MYD88 |
| Ciliated Cells | IQSEC1 | Ciliated Cells | FAM213B | Squamous Cells | CTTNBP2NL |
| Ciliated Cells | PNMA1 | Ciliated Cells | UPF3A | Squamous Cells | B2M |
| Ciliated Cells | ANKDD1B | Ciliated Cells | ALDH5A1 | Squamous Cells | LY6K |
| Ciliated Cells | DUBR | Ciliated Cells | USP40 | Squamous Cells | RAB27B |
| Ciliated Cells | UGDH | Ciliated Cells | CASP7 | Squamous Cells | QKI |
| Ciliated Cells | PIGQ | Ciliated Cells | QSOX2 | Squamous Cells | ANXA9 |
| Ciliated Cells | AGPAT3 | Ciliated Cells | GPC4 | Squamous Cells | MAPK3 |
| Ciliated Cells | NDUFV1 | Ciliated Cells | ZNF839 | Squamous Cells | RHOD |
| Ciliated Cells | MYO1B | Ciliated Cells | SHARPIN | Squamous Cells | C9orf16 |
| Ciliated Cells | SEMA3C | Ciliated Cells | HNRNPH3 | Squamous Cells | EHD1 |
| Ciliated Cells | MTCH1 | Ciliated Cells | CHURC1 | Squamous Cells | PLEKHF1 |
| Ciliated Cells | SLC4A11 | Ciliated Cells | RUFY2 | Squamous Cells | TMED3 |
| Ciliated Cells | EFCAB11 | Ciliated Cells | SLC38A10 | Squamous Cells | C4orf19 |
| Ciliated Cells | DUSP14 | Ciliated Cells | NUDT7 | Squamous Cells | ARHGAP27 |
| Ciliated Cells | ALDH9A1 | Ciliated Cells | LHPP | Squamous Cells | MAP3K9 |
| Ciliated Cells | RIPOR2 | Ciliated Cells | BECN1 | Squamous Cells | FAM89B |
| Ciliated Cells | SSRP1 | Ciliated Cells | TXLNG | Squamous Cells | EPHB3 |
| Ciliated Cells | RNPEP | Ciliated Cells | PEPD | Squamous Cells | ANKRD36C |
| Ciliated Cells | GLIPR2 | Ciliated Cells | APLP2 | Squamous Cells | IER2 |
| Ciliated Cells | BBS2 | Ciliated Cells | WEE1 | Squamous Cells | LYPD2 |
| Ciliated Cells | PPP1R42 | Ciliated Cells | SF3B1 | Squamous Cells | CDC34 |
| Ciliated Cells | EEF2K | Ciliated Cells | NDUFB5 | Squamous Cells | HIST1H2BC |
| Ciliated Cells | SS18 | Ciliated Cells | MPP7 | Squamous Cells | RNF149 |
| Ciliated Cells | KDM4A | Ciliated Cells | IDE | Squamous Cells | EPHX3 |
| Ciliated Cells | EMB | Ciliated Cells | LARS | Squamous Cells | MYEOV |
| Ciliated Cells | EIF2AK1 | Ciliated Cells | PPP2R3B | Squamous Cells | AGFG2 |
| Ciliated Cells | LAP3 | Ciliated Cells | OCIAD2 | Squamous Cells | VASN |
| Ciliated Cells | TMPRSS3 | Ciliated Cells | ZBTB33 | Squamous Cells | MAFG |
| Ciliated Cells | KCTD12 | Ciliated Cells | SNX4 | Squamous Cells | NR1D1 |
| Ciliated Cells | CBX5 | Ciliated Cells | SSBP3 | Squamous Cells | ABTB2 |
| Ciliated Cells | NAA20 | Ciliated Cells | HAX1 | Squamous Cells | MYZAP |
| Ciliated Cells | CEP44 | Ciliated Cells | ATIC | Squamous Cells | CNPPD1 |
| Ciliated Cells | CDK5RAP3 | Ciliated Cells | MDH1 | Squamous Cells | RARG |
| Ciliated Cells | SIX1 | Ciliated Cells | CYC1 | Squamous Cells | GRHL3 |
| Ciliated Cells | CCPG1 | Ciliated Cells | DLAT | Squamous Cells | HMOX1 |
| Ciliated Cells | SH3GLB2 | Ciliated Cells | CD2BP2 | Squamous Cells | LINC01559 |
| Ciliated Cells | DYNC2LI1 | Ciliated Cells | MYBBP1A | Squamous Cells | PIEZO1 |
| Ciliated Cells | USP43 | Ciliated Cells | TP53AIP1 | Squamous Cells | TRIM11 |
| Ciliated Cells | CIPC | Ciliated Cells | FAM122A | Squamous Cells | PAX6 |
| Ciliated Cells | BCO2 | Ciliated Cells | DGLUCY | Squamous Cells | 5-Mar |
| Ciliated Cells | SNX29 | Ciliated Cells | CEP120 | Squamous Cells | ARHGEF10L |
| Ciliated Cells | AGR3 | Ciliated Cells | PUDP | Squamous Cells | MERTK |
| Ciliated Cells | TRMT1L | Ciliated Cells | ZNF251 | Squamous Cells | MARVELD3 |
| Ciliated Cells | IQANK1 | Ciliated Cells | MRPL16 | Squamous Cells | B3GNT8 |
| Ciliated Cells | XRN2 | Ciliated Cells | ZMYND8 | Squamous Cells | CLIC3 |
| Ciliated Cells | MCAT | Ciliated Cells | AZIN1-AS1 | Squamous Cells | CDKN2A |
| Ciliated Cells | TRIM56 | Ciliated Cells | TMEM203 | Squamous Cells | FURIN |
| Ciliated Cells | BTBD9 | Ciliated Cells | KCTD11 | Squamous Cells | ABCG1 |
| Ciliated Cells | FAM120A | Ciliated Cells | FAM102A | Squamous Cells | TUBB6 |
| Ciliated Cells | ACBD3-AS1 | Ciliated Cells | ADH5 | Squamous Cells | KLK11 |
| Ciliated Cells | AC092802.1 | Ciliated Cells | BLVRA | Squamous Cells | REEP4 |
| Ciliated Cells | ZCWPW1 | Ciliated Cells | JAGN1 | Squamous Cells | TICAM1 |
| Ciliated Cells | GNS | Ciliated Cells | PRPF31 | Squamous Cells | PLEKHM1 |
| Ciliated Cells | KIAA1257 | Ciliated Cells | TSPYL5 | Squamous Cells | VWF |
| Ciliated Cells | SLC35A2 | Ciliated Cells | EIF31 | Squamous Cells | ACAD8 |
| Ciliated Cells | RABL6 | Ciliated Cells | LONP1 | Squamous Cells | LYPLA1 |
| Ciliated Cells | CCT7 | Ciliated Cells | TPST1 | Squamous Cells | DCLRE1C |
| Ciliated Cells | FSTL1 | Ciliated Cells | RITA1 | Squamous Cells | VSIG10 |
| Ciliated Cells | PCLO | Ciliated Cells | IWS1 | Squamous Cells | PLCD3 |
| Ciliated Cells | C3orf67 | Ciliated Cells | CCDC25 | Squamous Cells | MAF |
| Ciliated Cells | CRY2 | Ciliated Cells | C1QBP | Squamous Cells | PMEPA1 |
| Ciliated Cells | RRAGA | Ciliated Cells | AK1 | Squamous Cells | ZFP36 |
| Ciliated Cells | ARSD | Ciliated Cells | TOMM20 | Squamous Cells | RHBDF1 |
| Ciliated Cells | GRM7 | Dendritic Cells | HLA-DPB1 | Squamous Cells | AMOTL2 |
| Ciliated Cells | SMIM22 | Dendritic Cells | HLA-DRA | Squamous Cells | MPP5 |
| Ciliated Cells | MAP7 | Dendritic Cells | CD83 | Squamous Cells | ARHGAP10 |
| Ciliated Cells | GLYR1 | Dendritic Cells | HLA-DPA1 | Squamous Cells | RIT1 |
| Ciliated Cells | CAP2 | Dendritic Cells | CD74 | Squamous Cells | C15orf39 |
| Ciliated Cells | FXYD3 | Dendritic Cells | HLA-DRB1 | Squamous Cells | GBA |
| Ciliated Cells | PRPF4B | Deuterosomal Cells | CDC20B | Squamous Cells | RIN3 |
| Ciliated Cells | TCERG1 | Deuterosomal Cells | CCNO | Squamous Cells | KLF7 |
| Ciliated Cells | PRDX6 | Deuterosomal Cells | HES6 | Squamous Cells | RASA2 |
| Ciliated Cells | HSP90AA1 | Deuterosomal Cells | BTG3 | Squamous Cells | TMCC3 |
| Ciliated Cells | PER2 | Deuterosomal Cells | FOXN4 | Squamous Cells | NATD1 |
| Ciliated Cells | HGS | Deuterosomal Cells | PLK4 | Squamous Cells | CD68 |
| Ciliated Cells | HSBP1 | Developing Ciliated Cells | MMACHC | Squamous Cells | KCNK6 |
| Ciliated Cells | LPGAT1 | Developing Ciliated Cells | SAA1 | Squamous Cells | RUNX2 |
| Ciliated Cells | GOLPH3L | Developing Ciliated Cells | FAM166A | Squamous Cells | TSPAN14 |
| Ciliated Cells | ATP6V0A4 | Developing Ciliated Cells | BEST1 | Squamous Cells | RASAL1 |
| Ciliated Cells | ARHGEF28 | Developing Ciliated Cells | AC093484.3 | Squamous Cells | FXYD5 |
| Ciliated Cells | FAM206A | Developing Ciliated Cells | AGR3 | Squamous Cells | GPR153 |
| Ciliated Cells | NAP1L4 | Developing Ciliated Cells | AC006064.4 | Squamous Cells | INPP5K |
| Ciliated Cells | CFAP20 | Developing Ciliated Cells | CALM2 | Squamous Cells | DBP |
| Ciliated Cells | TMEM125 | Developing Ciliated Cells | COX6A1 | Squamous Cells | MROH6 |
| Ciliated Cells | MRPL40 | Developing Ciliated Cells | SAA2 | Squamous Cells | KLF6 |
| Ciliated Cells | YWHAE | Developing Ciliated Cells | PIFO | Squamous Cells | CCDC9B |
| Ciliated Cells | KANSL1L | Developing Ciliated Cells | RPL41 | Squamous Cells | PRAG1 |
| Ciliated Cells | NAT1 | Developing Ciliated Cells | TXN | Squamous Cells | MYO1C |
| Ciliated Cells | AMZ2 | Developing Ciliated Cells | CAPSL | Squamous Cells | CD59 |
| Ciliated Cells | IARS2 | Developing Ciliated Cells | PRKAR1A | Squamous Cells | VLDLR |
| Ciliated Cells | TPR | Developing Ciliated Cells | FAM183A | Squamous Cells | KCTD5 |
| Ciliated Cells | INPP5E | Developing Ciliated Cells | ARL3 | Squamous Cells | BMP1 |
| Ciliated Cells | ARHGAP42 | Developing Ciliated Cells | C11orf88 | Squamous Cells | BOK |
| Ciliated Cells | NRDC | Developing Ciliated Cells | CD24 | Squamous Cells | AKIRIN2 |
| Ciliated Cells | CCDC89 | Developing Ciliated Cells | UQCR11 | Squamous Cells | TSC22D4 |
| Ciliated Cells | ZBTB18 | Developing Ciliated Cells | YWHAE | Squamous Cells | RCC1 |
| Ciliated Cells | LRRFIP1 | Developing Ciliated Cells | ATP5F1E | Squamous Cells | ANO8 |
| Ciliated Cells | ENDOG | Developing Ciliated Cells | HSPB11 | Squamous Cells | PNPLA2 |
| Ciliated Cells | TCTEX1D2 | Developing Ciliated Cells | NDUFA4 | Squamous Cells | RDH13 |
| Ciliated Cells | REV3L | Developing Ciliated Cells | C12orf75 | Squamous Cells | NKIRAS2 |
| Ciliated Cells | DAD1 | Developing Ciliated Cells | COX6C | Squamous Cells | GTPBP2 |
| Ciliated Cells | POLD2 | Developing Ciliated Cells | AL357093.2 | Squamous Cells | ST3GAL1 |
| Ciliated Cells | POLD3 | Developing Ciliated Cells | MLF1 | Squamous Cells | AL031777.3 |
| Ciliated Cells | VCP | Developing Ciliated Cells | MRPS31 | Squamous Cells | HRH1 |
| Ciliated Cells | DCDC2 | Developing Ciliated Cells | S100A6 | Squamous Cells | ZSWIM4 |
| Ciliated Cells | GSTM2 | Developing Ciliated Cells | C9orf24 | Squamous Cells | CDC42EP1 |
| Ciliated Cells | SDC4 | Developing Ciliated Cells | SNTN | Squamous Cells | CPM |
| Ciliated Cells | ZC2HC1C | Developing Ciliated Cells | SKP1 | Squamous Cells | ELOVL6 |
| Ciliated Cells | ZNF295-AS1 | Developing Ciliated Cells | CFAP300 | Squamous Cells | PPCDC |
| Ciliated Cells | TRIM37 | Developing Ciliated Cells | AC020922.3 | Squamous Cells | AL512274.1 |
| Ciliated Cells | CEP97 | Developing Ciliated Cells | ARMC3 | Squamous Cells | ZYX |
| Ciliated Cells | WDR93 | Developing Ciliated Cells | SPAG16 | Squamous Cells | ZNF554 |
| Ciliated Cells | RBM5 | Developing Ciliated Cells | FAM216B | Squamous Cells | PDLIM2 |
| Ciliated Cells | TIMM10B | Developing Ciliated Cells | NDUFB1 | Squamous Cells | PDZD2 |
| Ciliated Cells | CBY1 | Developing Ciliated Cells | GON7 | Squamous Cells | JARID2 |
| Ciliated Cells | ELP1 | Developing Ciliated Cells | DYNLRB2 | Squamous Cells | BLNK |
| Ciliated Cells | COBL | Developing Ciliated Cells | SAXO2 | Squamous Cells | PPP1R13L |
| Ciliated Cells | MRNIP | Developing Ciliated Cells | MORF4L1 | Squamous Cells | TIAM1 |
| Ciliated Cells | UBR4 | Developing Ciliated Cells | ATP5MD | T Cells | TNFAIP3 |
| Ciliated Cells | ELK3 | Developing Ciliated Cells | TEX26 | T Cells | CCL5 |
| Ciliated Cells | RALGAPA2 | Developing Ciliated Cells | DPY30 | T Cells | PTPRC |
| Ciliated Cells | PLPP5 | Developing Ciliated Cells | NDUFA1 | T Cells | CD69 |
| Ciliated Cells | NSFL1C | Developing Ciliated Cells | DYNLT1 | T Cells | IL7R |
| Ciliated Cells | GALC | Developing Ciliated Cells | PFN2 | T Cells | STK17B |
| Ciliated Cells | ACYP1 | Developing Ciliated Cells | CALM1 | T Cells | CXCR4 |
| Ciliated Cells | GDI1 | Developing Ciliated Cells | PRDX1 | T Cells | NR4A2 |
| Ciliated Cells | SLC4A8 | Developing Ciliated Cells | SRP14 | T Cells | KLF6 |
| Ciliated Cells | ATXN2 | Developing Ciliated Cells | UGDH | T Cells | LCP1 |
| Ciliated Cells | THRAP3 | Developing Ciliated Cells | RPL34 | T Cells | TSC22D3 |
| Ciliated Cells | CRNDE | Developing Ciliated Cells | DSTN | T Cells | TAGAP |
| Ciliated Cells | LEKR1 | Developing Ciliated Cells | ANXA1 | T Cells | CD2 |
| Ciliated Cells | DYDC1 | Developing Ciliated Cells | COX7C | T Cells | ETS1 |
| Ciliated Cells | TAGLN2 | Developing Ciliated Cells | COX7B | T Cells | CD8A |
| Ciliated Cells | COA3 | Developing Ciliated Cells | HSPE1 | T Cells | TRBC2 |
| Ciliated Cells | APOO | Developing Ciliated Cells | PTGES3 | T Cells | ITGA1 |
| Ciliated Cells | SH3D19 | Developing Ciliated Cells | TCTEX1D2 | T Cells | RGS1 |
| Ciliated Cells | PRR18 | Developing Ciliated Cells | SERF2 | T Cells | SRGN |
| Ciliated Cells | CTBP2 | Developing Ciliated Cells | CDC42 | T Cells | BTG1 |
| Ciliated Cells | CASTOR3 | Developing Ciliated Cells | UQCRQ | T Cells | FYB1 |
| Ciliated Cells | AL121899.1 | Developing Ciliated Cells | HNRNPK | T Cells | CD96 |
| Ciliated Cells | NPEPL1 | Developing Ciliated Cells | PPP1CB | T Cells | FOSB |
| Ciliated Cells | PYCR2 | Developing Ciliated Cells | VAPA | T Cells | IKZF3 |
| Ciliated Cells | NT5DC1 | Developing Ciliated Cells | FTL | T Cells | CD3D |
| Ciliated Cells | GTF3C1 | Developing Ciliated Cells | SNX3 | T Cells | SAMSN1 |
| Ciliated Cells | UPF1 | Developing Ciliated Cells | B2M | T Cells | GZMB |
| Ciliated Cells | MUCL1 | Developing Ciliated Cells | PRDX5 | T Cells | ZFP36L2 |
| Ciliated Cells | FOCAD | Developing Ciliated Cells | HSP90AB1 | T Cells | CCND2 |
| Ciliated Cells | TIMP4 | Developing Ciliated Cells | MT-ND4L | T Cells | MCL1 |
| Ciliated Cells | CAPN9 | Developing Ciliated Cells | EZR | T Cells | IKZF1 |
| Ciliated Cells | TTC3 | Developing Ciliated Cells | TSPAN1 | T Cells | MBNL1 |
| Ciliated Cells | GMPR2 | Developing Ciliated Cells | FTH1 | T Cells | RGCC |
| Ciliated Cells | MSL3 | Developing Ciliated Cells | CAPS | T Cells | STK17A |
| Ciliated Cells | SH3RF3 | Developing Secretory and | ADAM28 | T Cells | RORA |
| | | Goblet Cells | | | |
| Ciliated Cells | KIAA1671 | Developing Secretory and | FAT2 | T Cells | TENT5C |
| | | Goblet Cells | | | |
| Ciliated Cells | FBXO36 | Developing Secretory and | SRRM2 | T Cells | SLA |
| | | Goblet Cells | | | |
| Ciliated Cells | HNRNPLL | Developing Secretory and | MALAT1 | T Cells | GBP5 |
| | | Goblet Cells | | | |
| Ciliated Cells | FAM120B | Enteroendocrine Cells | TBC1D9B | T Cells | DNAJB1 |
| Ciliated Cells | UVSSA | Erythroblasts | HBB | T Cells | TRBC1 |
| Ciliated Cells | ADSS | Erythroblasts | HBA2 | T Cells | LCP2 |
| Ciliated Cells | FUBP1 | Erythroblasts | MTRNR2L1 | T Cells | PTPN22 |
| Ciliated Cells | DEGS2 | Erythroblasts | MTRNR2L12 | T Cells | TSPYL2 |
| Ciliated Cells | IDH2 | Erythroblasts | MT-ND3 | T Cells | VIM |
| Ciliated Cells | DIDO1 | Erythroblasts | MT-CO1 | T Cells | HSPH1 |
| Ciliated Cells | BDH1 | Erythroblasts | MT-ND1 | T Cells | DOCK10 |
| Ciliated Cells | C11orf74 | Erythroblasts | MT-ND5 | T Cells | KIAA1551 |
| Ciliated Cells | EGLN3 | Erythroblasts | MT-CYB | T Cells | IFNG |
| Ciliated Cells | GSTA3 | Erythroblasts | MT-ATP6 | T Cells | NR4A1 |
| Ciliated Cells | CASC4 | Erythroblasts | MT-ND2 | T Cells | SPOCK2 |
| Ciliated Cells | C22orf23 | Erythroblasts | MT-ND4 | T Cells | TRAC |
| Ciliated Cells | PFDN5 | Goblet Cells | MUC5AC | T Cells | CORO1A |
| Ciliated Cells | MANBAL | Goblet Cells | PI3 | T Cells | NR4A3 |
| Ciliated Cells | IP6K1 | Goblet Cells | BPIFB1 | T Cells | WIPF1 |
| Ciliated Cells | TMEM234 | Goblet Cells | VMO1 | T Cells | MSN |
| Ciliated Cells | AES | Goblet Cells | SERPINB3 | T Cells | PTGER4 |
| Ciliated Cells | TUBGCP6 | Goblet Cells | WFDC2 | T Cells | CLEC2D |
| Ciliated Cells | ADSSL1 | Goblet Cells | RARRES1 | T Cells | IRF4 |
| Ciliated Cells | DHCR24 | Goblet Cells | PSCA | T Cells | GPRIN3 |
| Ciliated Cells | SLC38A6 | Goblet Cells | AQP5 | T Cells | DUSP2 |
| Ciliated Cells | GOLGA8B | Goblet Cells | SLPI | T Cells | MYADM |
| Ciliated Cells | B3GNT7 | Goblet Cells | MSMB | T Cells | RGS2 |
| Ciliated Cells | CDC5L | Goblet Cells | S100P | T Cells | BRD2 |
| Ciliated Cells | DDB1 | Goblet Cells | EPAS1 | T Cells | ELF1 |
| Ciliated Cells | EFEMP1 | Goblet Cells | LCN2 | T Cells | PREX1 |
| Ciliated Cells | TMEM94 | Goblet Cells | C3 | T Cells | ZC3HAV1 |
| Ciliated Cells | ERN2 | Goblet Cells | LYPD2 | T Cells | CD3G |
| Ciliated Cells | DDX24 | Goblet Cells | PRSS23 | T Cells | SLC2A3 |
| Ciliated Cells | WASHC2A | Goblet Cells | PIGR | T Cells | ARHGAP30 |
| Ciliated Cells | SLC2A1 | Goblet Cells | KRT19 | T Cells | FLNA |
| Ciliated Cells | CCT3 | Goblet Cells | MSLN | T Cells | SRSF7 |
| Ciliated Cells | MDM2 | Goblet Cells | ADH1C | T Cells | JUND |
| Ciliated Cells | SUDS3 | Goblet Cells | F3 | T Cells | BTG2 |
| Ciliated Cells | PTGES3 | Goblet Cells | FAM3D | T Cells | SRSF3 |
| Ciliated Cells | SUGP2 | Goblet Cells | MUC1 | T Cells | JAML |
| Ciliated Cells | TNFRSF14- | Goblet Cells | AGR2 | T Cells | LAPTM5 |
| | AS1 | | | | |
| Ciliated Cells | FBXW10 | Goblet Cells | XBP1 | T Cells | NEU1 |
| Ciliated Cells | BPTF | Goblet Cells | CXCL17 | T Cells | CELF2 |
| Ciliated Cells | TTBK2 | Goblet Cells | CAPN13 | T Cells | CNOT6L |
| Ciliated Cells | ATP5F1A | Goblet Cells | ANKRD36C | T Cells | PAG1 |
| Ciliated Cells | NPHP4 | Goblet Cells | CP | T Cells | PNRC1 |
| Ciliated Cells | GBF1 | Goblet Cells | CYP2F1 | T Cells | ITGA4 |
| Ciliated Cells | MID1 | Goblet Cells | KRT7 | T Cells | FYN |
| Ciliated Cells | TACC2 | Goblet Cells | MMP10 | T Cells | HECA |
| Ciliated Cells | LDHB | Goblet Cells | CRACR2B | T Cells | ACAP1 |
| Ciliated Cells | EXTL2 | Goblet Cells | FER1L6 | T Cells | JUN |
| Ciliated Cells | PRDX3 | Goblet Cells | ERN2 | T Cells | DNAJA1 |
| Ciliated Cells | CYB5R1 | Goblet Cells | RDH10 | T Cells | LCK |
| Ciliated Cells | DHX32 | Goblet Cells | ALDH3A1 | T Cells | GZMA |
| Ciliated Cells | CHMP2A | Goblet Cells | ASRGL1 | T Cells | PARP8 |
| Ciliated Cells | CLCN3 | Goblet Cells | ADAM28 | T Cells | ZFP36L1 |
| Ciliated Cells | NIPSNAP2 | Goblet Cells | XIST | T Cells | RUNX3 |
| Ciliated Cells | RALGPS2 | Goblet Cells | GABRP | T Cells | RNF213 |
| Ciliated Cells | CATIP | Goblet Cells | STEAP4 | T Cells | BCL11B |
| Ciliated Cells | NAV2 | Goblet Cells | DUOX2 | T Cells | IVNS1ABP |
| Ciliated Cells | TMEM9 | Goblet Cells | KRT4 | T Cells | ITGAL |
| Ciliated Cells | RHOBTB1 | Goblet Cells | NOS2 | T Cells | GLS |
| Ciliated Cells | SNCAIP | Goblet Cells | CTSD | T Cells | MYH9 |
| Ciliated Cells | SIX4 | Goblet Cells | GRN | T Cells | STK10 |
| Ciliated Cells | EHF | Goblet Cells | CDC42BPA | T Cells | SERPINB9 |
| Ciliated Cells | CFAP77 | Goblet Cells | CD55 | T Cells | NLRC3 |
| Ciliated Cells | HNRNPA2B1 | Goblet Cells | ATP1B1 | T Cells | PPP1R15B |
| Ciliated Cells | MDN1 | Goblet Cells | TMEM213 | T Cells | SLC38A1 |
| Ciliated Cells | ZNF606 | Goblet Cells | MALAT1 | T Cells | CD44 |
| Ciliated Cells | CERS6 | Goblet Cells | SLC6A14 | T Cells | LBH |
| Ciliated Cells | FBXL5 | Goblet Cells | NTS | T Cells | CYTIP |
| Ciliated Cells | ACOX3 | Goblet Cells | GSTK1 | T Cells | ARL4C |
| Ciliated Cells | CCT6A | Goblet Cells | MUC4 | T Cells | ARHGAP9 |
| Ciliated Cells | CCDC28A | Goblet Cells | CHD3 | T Cells | PIP4K2A |
| Ciliated Cells | SPG11 | Goblet Cells | TFCP2L1 | T Cells | IL2RB |
| Ciliated Cells | KCNH3 | Goblet Cells | FAM129A | T Cells | SUN2 |
| Ciliated Cells | CNPY3 | Goblet Cells | A4GALT | T Cells | CD53 |
| Ciliated Cells | EID1 | Goblet Cells | TNFSF10 | T Cells | ARHGAP45 |
| Ciliated Cells | ICMT | Goblet Cells | ST6GAL1 | T Cells | TBC1D10C |
| Ciliated Cells | C19orf44 | Goblet Cells | TSPAN13 | T Cells | DDX3X |
| Ciliated Cells | BAG6 | Goblet Cells | UGT2A1 | T Cells | GNG2 |
| Ciliated Cells | KIF24 | Goblet Cells | ASS1 | T Cells | ODC1 |
| Ciliated Cells | ENO2 | Goblet Cells | FUT2 | T Cells | SEMA4D |
| Ciliated Cells | MZF1 | Goblet Cells | RIMS1 | T Cells | ICOS |
| Ciliated Cells | FAM219B | Goblet Cells | AKR1C1 | T Cells | FNBP1 |
| Ciliated Cells | ZNF3 | Goblet Cells | STARD10 | T Cells | TRAF3IP3 |
| Ciliated Cells | FEZF1-AS1 | Goblet Cells | RRBP1 | T Cells | TMC8 |
| Ciliated Cells | PACSIN2 | Goblet Cells | AZGP1 | T Cells | SMAD7 |
| Ciliated Cells | ATP6V1D | Goblet Cells | HSPB1 | T Cells | DOCK8 |
| Ciliated Cells | NBAS | Goblet Cells | HLA-A | T Cells | STK4 |
| Ciliated Cells | SSBP2 | Goblet Cells | FBP1 | T Cells | ITK |
| Ciliated Cells | IL16 | Goblet Cells | TMEM184A | T Cells | PPP1R15A |
| Ciliated Cells | RB1CC1 | Goblet Cells | S100A14 | T Cells | CD52 |
| Ciliated Cells | SLC13A3 | Goblet Cells | CAPN8 | T Cells | RPS19 |
| Ciliated Cells | OGFOD2 | Goblet Cells | TMPRSS4 | T Cells | APBB1IP |
| Ciliated Cells | PSMC5 | Goblet Cells | S100A4 | T Cells | SRSF2 |
| Ciliated Cells | FAM8A1 | Goblet Cells | ALPL | T Cells | RPS3 |
| Ciliated Cells | AK8 | Goblet Cells | CEACAM5 | T Cells | DEDD2 |
| Ciliated Cells | LINC01571 | Goblet Cells | S100A6 | T Cells | PPP1R16B |
| Ciliated Cells | HIBADH | Goblet Cells | ANPEP | T Cells | HLA-E |
| Ciliated Cells | DNER | Goblet Cells | NEAT1 | T Cells | RAC2 |
| Ciliated Cells | CLTC | Goblet Cells | FCGBP | T Cells | CD3E |
| Ciliated Cells | ARFGAP1 | Goblet Cells | VSIG2 | T Cells | BIN2 |
| Ciliated Cells | SMARCA2 | Goblet Cells | ME3 | T Cells | DOK2 |
| Ciliated Cells | ETNK1 | Goblet Cells | TCEA3 | T Cells | CD247 |
| Ciliated Cells | IQCB1 | Goblet Cells | AQP3 | T Cells | KLRB1 |
| Ciliated Cells | RPA3 | Goblet Cells | CYP2J2 | T Cells | DOCK2 |
| Ciliated Cells | OFD1 | Goblet Cells | GOLGA4 | T Cells | ARHGAP4 |
| Ciliated Cells | NDUFA8 | Goblet Cells | IFITM3 | T Cells | GIMAP7 |
| Ciliated Cells | PPP6R1 | Goblet Cells | MLPH | T Cells | RASGRP1 |
| Ciliated Cells | DLG3 | Goblet Cells | SCNN1A | T Cells | ATF4 |
| Ciliated Cells | MNAT1 | Goblet Cells | KCNE3 | T Cells | ZAP70 |
| Ciliated Cells | U2SURP | Goblet Cells | GLUL | T Cells | IL32 |
| Ciliated Cells | C2orf50 | Goblet Cells | MUC16 | T Cells | IL10RA |
| Ciliated Cells | TMEM254 | Goblet Cells | CD151 | T Cells | CD6 |
| Ciliated Cells | ANKRD13D | Goblet Cells | VAMP8 | T Cells | RHOH |
| Ciliated Cells | KIF5B | Goblet Cells | EPS8L1 | T Cells | SPN |
| Ciliated Cells | NUP50-DT | Goblet Cells | SELENBP1 | T Cells | PRKACB |
| Ciliated Cells | RABGAP1L | Goblet Cells | CLINT1 | T Cells | CD226 |
| Ciliated Cells | LRRC18 | Goblet Cells | KLF5 | T Cells | CD48 |
| Ciliated Cells | CCDC171 | Goblet Cells | SLC15A2 | T Cells | CCR5 |
| Ciliated Cells | WDR31 | Goblet Cells | CRYM | T Cells | ARID5A |
| Ciliated Cells | CACNG6 | Goblet Cells | S100A16 | T Cells | CAMK4 |
| Ciliated Cells | LTK | Goblet Cells | LMO4 | T Cells | FMNL1 |
| Ciliated Cells | HK1 | Goblet Cells | CD36 | T Cells | TNFRSF1B |
| Ciliated Cells | TUB | Goblet Cells | RPL8 | T Cells | PSTPIP1 |
| Ciliated Cells | AC113349.1 | Goblet Cells | CFH | T Cells | SCML4 |
| Ciliated Cells | DDAH1 | Goblet Cells | DHCR24 | T Cells | RASAL3 |
| Ciliated Cells | CD200R1L | Goblet Cells | SPDEF | T Cells | ZNF683 |
| Ciliated Cells | SLC23A2 | Goblet Cells | RARRES3 | T Cells | SFMBT2 |
| Ciliated Cells | PDIA4 | Goblet Cells | PLEKHH1 | T Cells | KLRC1 |
| Ciliated Cells | TTC23L | Goblet Cells | ZNHIT6 | T Cells | MYO1F |
| Ciliated Cells | RERE | Goblet Cells | GOLM1 | T Cells | AOAH |
| Ciliated Cells | CCDC93 | Goblet Cells | AKR1C2 | T Cells | TRGC2 |
| Ciliated Cells | KCNRG | Goblet Cells | CEACAM6 | T Cells | ADCY7 |
| Ciliated Cells | PXN | Goblet Cells | RPL18 | T Cells | RCSD1 |
| Ciliated Cells | BRD3OS | Goblet Cells | CST3 | T Cells | SASH3 |
| Ciliated Cells | TSPYL4 | Goblet Cells | LY6E | T Cells | TBX21 |
| Ciliated Cells | COL9A2 | Goblet Cells | CREB3L1 | T Cells | SEPT1 |
| Ciliated Cells | ZDHHC1 | Goblet Cells | LGALS3BP | T Cells | STAT4 |
| Ciliated Cells | PWWP2B | Goblet Cells | GPX2 | T Cells | GPR174 |
| Ciliated Cells | AHCYL1 | Goblet Cells | SRRM2 | T Cells | SLAMF6 |
| Ciliated Cells | CLTA | Goblet Cells | GSN | T Cells | JAK3 |
| Ciliated Cells | CDC42BPB | Goblet Cells | LYNX1 | T Cells | NKG7 |
| Ciliated Cells | STXBP4 | Goblet Cells | SYNGR2 | T Cells | PRKCQ |
| Ciliated Cells | RIMKLB | Goblet Cells | ST6GALNAC1 | T Cells | CD7 |

Table 1B. Detailed Epithelial Cell Types (see FIG. 2)

| | | | | | |
|---|---|---|---|---|---|
| AZGP1 high Goblet Cells | PI3 | Early Response FOXJ1 high | PIH1D3 | FOXJ1 high Ciliated Cells | MAOB |
| | | Ciliated Cells | | | |
| AZGP1 high Goblet Cells | PSCA | Early Response FOXJ1 high | COBL | FOXJ1 high Ciliated Cells | TTC26 |
| | | Ciliated Cells | | | |
| AZGP1 high Goblet Cells | VMO1 | Early Response FOXJ1 high | CCDC60 | FOXJ1 high Ciliated Cells | TTC12 |
| | | Ciliated Cells | | | |
| AZGP1 high Goblet Cells | S100P | Early Response FOXJ1 high | EPB41L1 | FOXJ1 high Ciliated Cells | TRMT9B |
| | | Ciliated Cells | | | |
| AZGP1 high Goblet Cells | WFDC2 | Early Response FOXJ1 high | CCDC189 | FOXJ1 high Ciliated Cells | PFN2 |
| | | Ciliated Cells | | | |
| AZGP1 high Goblet Cells | RARRES1 | Early Response FOXJ1 high | BAIAP2-DT | FOXJ1 high Ciliated Cells | DYNLRB2 |
| | | Ciliated Cells | | | |
| AZGP1 high Goblet Cells | AQP5 | Early Response FOXJ1 high | EFNB3 | FOXJ1 high Ciliated Cells | SPTLC2 |
| | | Ciliated Cells | | | |
| AZGP1 high Goblet Cells | SERPINB3 | Early Response FOXJ1 high | CLCN3 | FOXJ1 high Ciliated Cells | TTLL9 |
| | | Ciliated Cells | | | |
| AZGP1 high Goblet Cells | BPIFB1 | Early Response FOXJ1 high | DALRD3 | FOXJ1 high Ciliated Cells | SLFN13 |
| | | Ciliated Cells | | | |
| AZGP1 high Goblet Cells | LCN2 | Early Response FOXJ1 high | KIAA1671 | FOXJ1 high Ciliated Cells | MAPK8IP1 |
| | | Ciliated Cells | | | |
| AZGP1 high Goblet Cells | MUC5AC | Early Response FOXJ1 high | TCTN2 | FOXJ1 high Ciliated Cells | WDR34 |
| | | Ciliated Cells | | | |
| AZGP1 high Goblet Cells | MSLN | Early Response FOXJ1 high | APOO | FOXJ1 high Ciliated Cells | MPC2 |
| | | Ciliated Cells | | | |
| AZGP1 high Goblet Cells | ALDH3A1 | Early Response FOXJ1 high | CIPC | FOXJ1 high Ciliated Cells | CASC1 |
| | | Ciliated Cells | | | |
| AZGP1 high Goblet Cells | FAM3D | Early Response FOXJ1 high | FGD5-AS1 | FOXJ1 high Ciliated Cells | FBXO15 |
| | | Ciliated Cells | | | |
| AZGP1 high Goblet Cells | MSMB | Early Response FOXJ1 high | EIF3D | FOXJ1 high Ciliated Cells | TCTN2 |
| | | Ciliated Cells | | | |
| AZGP1 high Goblet Cells | C3 | Early Response FOXJ1 high | ATP2B4 | FOXJ1 high Ciliated Cells | WRB |
| | | Ciliated Cells | | | |
| AZGP1 high Goblet Cells | KRT19 | Early Response FOXJ1 high | RAB11FIP4 | FOXJ1 high Ciliated Cells | WFDC6 |
| | | Ciliated Cells | | | |
| AZGP1 high Goblet Cells | LYPD2 | Early Response FOXJ1 high | GABARAPL2 | FOXJ1 high Ciliated Cells | CFAP47 |
| | | Ciliated Cells | | | |
| AZGP1 high Goblet Cells | AGR2 | Early Response FOXJ1 high | FAM13A | FOXJ1 high Ciliated Cells | DZIP1L |
| | | Ciliated Cells | | | |
| AZGP1 high Goblet Cells | XBP1 | Early Response FOXJ1 high | UNC93B1 | FOXJ1 high Ciliated Cells | TMC4 |
| | | Ciliated Cells | | | |
| AZGP1 high Goblet Cells | DUOX2 | Early Response FOXJ1 high | TNFRSF21 | FOXJ1 high Ciliated Cells | NSUN7 |
| | | Ciliated Cells | | | |
| AZGP1 high Goblet Cells | MUC1 | Early Response FOXJ1 high | CCDC157 | FOXJ1 high Ciliated Cells | MTURN |
| | | Ciliated Cells | | | |
| AZGP1 high Goblet Cells | S100A9 | Early Response FOXJ1 high | AKAP14 | FOXJ1 high Ciliated Cells | ZBBX |
| | | Ciliated Cells | | | |
| AZGP1 high Goblet Cells | FCGBP | Early Response FOXJ1 high | CTSH | FOXJ1 high Ciliated Cells | ENKD1 |
| | | Ciliated Cells | | | |
| AZGP1 high Goblet Cells | KRT7 | Early Response FOXJ1 high | ZKSCAN1 | FOXJ1 high Ciliated Cells | CGN |
| | | Ciliated Cells | | | |
| AZGP1 high Goblet Cells | SLPI | Early Response FOXJ1 high | PPP2CB | FOXJ1 high Ciliated Cells | FILIP1 |
| | | Ciliated Cells | | | |
| AZGP1 high Goblet Cells | KRT4 | Early Response FOXJ1 high | TCTA | FOXJ1 high Ciliated Cells | DUSP18 |
| | | Ciliated Cells | | | |
| AZGP1 high Goblet Cells | CTSD | Early Response FOXJ1 high | PRKAR2A | FOXJ1 high Ciliated Cells | CFAP299 |
| | | Ciliated Cells | | | |
| AZGP1 high Goblet Cells | ASRGL1 | Early Response FOXJ1 high | DHX40 | FOXJ1 high Ciliated Cells | SLC23A1 |
| | | Ciliated Cells | | | |
| AZGP1 high Goblet Cells | ALPL | Early Response FOXJ1 high | TMEM59 | FOXJ1 high Ciliated Cells | WDR38 |
| | | Ciliated Cells | | | |
| AZGP1 high Goblet Cells | AZGP1 | Early Response FOXJ1 high | TRIM2 | FOXJ1 high Ciliated Cells | DNAI2 |
| | | Ciliated Cells | | | |
| AZGP1 high Goblet Cells | PRSS23 | Early Response FOXJ1 high | TMPRSS3 | FOXJ1 high Ciliated Cells | CES2 |
| | | Ciliated Cells | | | |
| AZGP1 high Goblet Cells | RDH10 | Early Response FOXJ1 high | ISCU | FOXJ1 high Ciliated Cells | CYSTM1 |
| | | Ciliated Cells | | | |
| AZGP1 high Goblet Cells | CEACAM5 | Early Response FOXJ1 high | ATP1A1 | FOXJ1 high Ciliated Cells | CCDC151 |
| | | Ciliated Cells | | | |
| AZGP1 high Goblet Cells | F3 | Early Response FOXJ1 high | MEA1 | FOXJ1 high Ciliated Cells | KIF9 |
| | | Ciliated Cells | | | |
| AZGP1 high Goblet Cells | EPAS1 | Early Response FOXJ1 high | MYB | FOXJ1 high Ciliated Cells | TMC5 |
| | | Ciliated Cells | | | |
| AZGP1 high Goblet Cells | GRN | Early Response FOXJ1 high | SPATA13 | FOXJ1 high Ciliated Cells | SYTL3 |
| | | Ciliated Cells | | | |
| AZGP1 high Goblet Cells | FBP1 | Early Response FOXJ1 high | CCT5 | FOXJ1 high Ciliated Cells | CD38 |
| | | Ciliated Cells | | | |
| AZGP1 high Goblet Cells | ADH1C | Early Response FOXJ1 high | ESYT2 | FOXJ1 high Ciliated Cells | GON7 |
| | | Ciliated Cells | | | |
| AZGP1 high Goblet Cells | IFITM3 | Early Response FOXJ1 high | HK1 | FOXJ1 high Ciliated Cells | STRBP |
| | | Ciliated Cells | | | |
| AZGP1 high Goblet Cells | CYP2F1 | Early Response FOXJ1 high | C22orf23 | FOXJ1 high Ciliated Cells | CFAP69 |
| | | Ciliated Cells | | | |
| AZGP1 high Goblet Cells | DUOXA2 | Early Response FOXJ1 high | TMEM125 | FOXJ1 high Ciliated Cells | TMEM212 |
| | | Ciliated Cells | | | |
| AZGP1 high Goblet Cells | PIGR | Early Response FOXJ1 high | SNX17 | FOXJ1 high Ciliated Cells | IFT43 |
| | | Ciliated Cells | | | |
| AZGP1 high Goblet Cells | CEACAM6 | Early Response FOXJ1 high | RNF130 | FOXJ1 high Ciliated Cells | ADGB |
| | | Ciliated Cells | | | |
| AZGP1 high Goblet Cells | FUT2 | Early Response FOXJ1 high | SMIM14 | FOXJ1 high Ciliated Cells | SQLE |
| | | Ciliated Cells | | | |
| AZGP1 high Goblet Cells | CD36 | Early Response FOXJ1 high | MDH1B | FOXJ1 high Ciliated Cells | SPEF2 |
| | | Ciliated Cells | | | |
| AZGP1 high Goblet Cells | STARD10 | Early Response FOXJ1 high | TEKT2 | FOXJ1 high Ciliated Cells | RHPN1 |
| | | Ciliated Cells | | | |
| AZGP1 high Goblet Cells | ST6GAL1 | Early Response FOXJ1 high | SEMA3C | FOXJ1 high Ciliated Cells | AP2B1 |
| | | Ciliated Cells | | | |
| AZGP1 high Goblet Cells | VSIG2 | Early Response FOXJ1 high | FAM210B | FOXJ1 high Ciliated Cells | PRRT3 |
| | | Ciliated Cells | | | |
| AZGP1 high Goblet Cells | GSTK1 | Early Response FOXJ1 high | C9orf72 | FOXJ1 high Ciliated Cells | CEP126 |
| | | Ciliated Cells | | | |
| AZGP1 high Goblet Cells | VAMP8 | Early Response FOXJ1 high | WDR31 | FOXJ1 high Ciliated Cells | ATP2A2 |
| | | Ciliated Cells | | | |
| AZGP1 high Goblet Cells | CXCL17 | Early Response FOXJ1 high | NSMCE1 | FOXJ1 high Ciliated Cells | DHRS9 |
| | | Ciliated Cells | | | |
| AZGP1 high Goblet Cells | P4HB | Early Response FOXJ1 high | C20orf96 | FOXJ1 high Ciliated Cells | PIH1D3 |
| | | Ciliated Cells | | | |
| AZGP1 high Goblet Cells | S100A16 | Early Response FOXJ1 high | ERLIN2 | FOXJ1 high Ciliated Cells | ARHGAP39 |
| | | Ciliated Cells | | | |
| AZGP1 high Goblet Cells | SCD | Early Response FOXJ1 high | CBY1 | FOXJ1 high Ciliated Cells | ZSCAN18 |
| | | Ciliated Cells | | | |
| AZGP1 high Goblet Cells | RARRES3 | Early Response FOXJ1 high | EPCAM | FOXJ1 high Ciliated Cells | GNAS |
| | | Ciliated Cells | | | |
| AZGP1 high Goblet Cells | ASS1 | Early Response FOXJ1 high | CASC4 | FOXJ1 high Ciliated Cells | TTC21A |
| | | Ciliated Cells | | | |
| AZGP1 high Goblet Cells | HLA-A | Early Response FOXJ1 high | MRFAP1 | FOXJ1 high Ciliated Cells | TUFM |
| | | Ciliated Cells | | | |
| AZGP1 high Goblet Cells | S100A4 | Early Response FOXJ1 high | MTCH1 | FOXJ1 high Ciliated Cells | SCPEP1 |
| | | Ciliated Cells | | | |
| AZGP1 high Goblet Cells | KCNE3 | Early Response FOXJ1 high | AMZ2 | FOXJ1 high Ciliated Cells | MORN3 |
| | | Ciliated Cells | | | |
| AZGP1 high Goblet Cells | IFITM2 | Early Response FOXJ1 high | CREBL2 | FOXJ1 high Ciliated Cells | CCDC24 |
| | | Ciliated Cells | | | |
| AZGP1 high Goblet Cells | TSPAN13 | Early Response FOXJ1 high | IFT172 | FOXJ1 high Ciliated Cells | TTC16 |
| | | Ciliated Cells | | | |
| AZGP1 high Goblet Cells | SPDEF | Early Response FOXJ1 high | DNAL4 | FOXJ1 high Ciliated Cells | PIH1D2 |
| | | Ciliated Cells | | | |
| AZGP1 high Goblet Cells | STEAP4 | Early Response FOXJ1 high | LRRC71 | FOXJ1 high Ciliated Cells | CFAP44 |
| | | Ciliated Cells | | | |
| AZGP1 high Goblet Cells | S100A14 | Early Response FOXJ1 high | TCP1 | FOXJ1 high Ciliated Cells | FRMPD2 |
| | | Ciliated Cells | | | |
| AZGP1 high Goblet Cells | GPX2 | Early Response FOXJ1 high | DEDD2 | FOXJ1 high Ciliated Cells | GAS8 |
| | | Ciliated Cells | | | |
| AZGP1 high Goblet Cells | PGD | Early Response FOXJ1 high | CLTC | FOXJ1 high Ciliated Cells | SLC20A2 |
| | | Ciliated Cells | | | |
| AZGP1 high Goblet Cells | CD55 | Early Response FOXJ1 high | FMN1 | FOXJ1 high Ciliated Cells | BTBD3 |
| | | Ciliated Cells | | | |
| AZGP1 high Goblet Cells | ATP1B1 | Early Response FOXJ1 high | RRAGA | FOXJ1 high Ciliated Cells | CLDN3 |
| | | Ciliated Cells | | | |
| AZGP1 high Goblet Cells | A4GALT | Early Response FOXJ1 high | NSFL1C | FOXJ1 high Ciliated Cells | GCLC |
| | | Ciliated Cells | | | |
| AZGP1 high Goblet Cells | SLC6A14 | Early Response FOXJ1 high | GFPT1 | FOXJ1 high Ciliated Cells | SRD5A2 |
| | | Ciliated Cells | | | |
| AZGP1 high Goblet Cells | LDHA | Early Response FOXJ1 high | PPM1H | FOXJ1 high Ciliated Cells | ANKRD42 |
| | | Ciliated Cells | | | |
| AZGP1 high Goblet Cells | CRACR2B | Early Response FOXJ1 high | IDH2 | FOXJ1 high Ciliated Cells | TPPP |
| | | Ciliated Cells | | | |
| AZGP1 high Goblet Cells | TSPO | Early Response FOXJ1 high | PRDX3 | FOXJ1 high Ciliated Cells | TUBGCP2 |
| | | Ciliated Cells | | | |
| AZGP1 high Goblet Cells | CREB3L1 | Early Response FOXJ1 high | LRRC49 | FOXJ1 high Ciliated Cells | PTPRT |
| | | Ciliated Cells | | | |
| AZGP1 high Goblet Cells | RAB37 | Early Response FOXJ1 high | PHTF1 | FOXJ1 high Ciliated Cells | KCTD12 |
| | | Ciliated Cells | | | |
| AZGP1 high Goblet Cells | CP | Early Response FOXJ1 high | ICA1L | FOXJ1 high Ciliated Cells | ATP9A |
| | | Ciliated Cells | | | |
| AZGP1 high Goblet Cells | RPL8 | Early Response FOXJ1 high | IDS | FOXJ1 high Ciliated Cells | LRIG1 |
| | | Ciliated Cells | | | |
| AZGP1 high Goblet Cells | CST3 | Early Response FOXJ1 high | CCDC24 | FOXJ1 high Ciliated Cells | CD164L2 |
| | | Ciliated Cells | | | |
| AZGP1 high Goblet Cells | PTGES | Early Response FOXJ1 high | DNAAF3 | FOXJ1 high Ciliated Cells | CLU |
| | | Ciliated Cells | | | |
| AZGP1 high Goblet Cells | S100A8 | Early Response FOXJ1 high | SOX9 | FOXJ1 high Ciliated Cells | CDKL1 |
| | | Ciliated Cells | | | |
| AZGP1 high Goblet Cells | HLA-DRB5 | Early Response FOXJ1 high | SAMD15 | FOXJ1 high Ciliated Cells | ARMC2 |
| | | Ciliated Cells | | | |
| AZGP1 high Goblet Cells | CAPN13 | Early Response FOXJ1 high | SPR | FOXJ1 high Ciliated Cells | APBB1 |
| | | Ciliated Cells | | | |
| AZGP1 high Goblet Cells | CD151 | Early Response FOXJ1 high | ARL13B | FOXJ1 high Ciliated Cells | SFXN3 |
| | | Ciliated Cells | | | |
| AZGP1 high Goblet Cells | S100A6 | Early Response FOXJ1 high | CATSPERD | FOXJ1 high Ciliated Cells | MUC15 |
| | | Ciliated Cells | | | |
| AZGP1 high Goblet Cells | GLUL | Early Response FOXJ1 high | PITPNA | FOXJ1 high Ciliated Cells | BBS4 |
| | | Ciliated Cells | | | |
| AZGP1 high Goblet Cells | CTSB | Early Response FOXJ1 high | SERTAD1 | FOXJ1 high Ciliated Cells | TMEM67 |
| | | Ciliated Cells | | | |
| AZGP1 high Goblet Cells | CFB | Early Response FOXJ1 high | CCNDBP1 | FOXJ1 high Ciliated Cells | SLC25A36 |
| | | Ciliated Cells | | | |
| AZGP1 high Goblet Cells | CRYM | Early Response FOXJ1 high | LMLN | FOXJ1 high Ciliated Cells | IFT88 |
| | | Ciliated Cells | | | |
| AZGP1 high Goblet Cells | GOLM1 | Early Response FOXJ1 high | NIPSNAP2 | FOXJ1 high Ciliated Cells | VDAC3 |
| | | Ciliated Cells | | | |
| AZGP1 high Goblet Cells | PKM | Early Response FOXJ1 high | FGF14 | FOXJ1 high Ciliated Cells | EFCAB12 |
| | | Ciliated Cells | | | |
| AZGP1 high Goblet Cells | HSPB1 | Early Response FOXJ1 high | MSI2 | FOXJ1 high Ciliated Cells | C4orf3 |
| | | Ciliated Cells | | | |
| AZGP1 high Goblet Cells | SORD | Early Response FOXJ1 high | STPG1 | FOXJ1 high Ciliated Cells | ARFGEF3 |
| | | Ciliated Cells | | | |
| AZGP1 high Goblet Cells | GABRP | Early Response FOXJ1 high | DNAH2 | FOXJ1 high Ciliated Cells | CCDC39 |
| | | Ciliated Cells | | | |
| AZGP1 high Goblet Cells | TMSB10 | Early Response FOXJ1 high | UGDH | FOXJ1 high Ciliated Cells | CCDC88C |
| | | Ciliated Cells | | | |
| AZGP1 high Goblet Cells | GAPDH | Early Response FOXJ1 high | SPRYD3 | FOXJ1 high Ciliated Cells | TMEM123 |
| | | Ciliated Cells | | | |
| AZGP1 high Goblet Cells | LY6E | Early Response FOXJ1 high | TMEM212 | FOXJ1 high Ciliated Cells | ACO2 |
| | | Ciliated Cells | | | |
| AZGP1 high Goblet Cells | LGALS3BP | Early Response FOXJ1 high | CERS6 | FOXJ1 high Ciliated Cells | TPRG1L |
| | | Ciliated Cells | | | |
| AZGP1 high Goblet Cells | BLVRB | Early Response FOXJ1 high | C11orf49 | FOXJ1 high Ciliated Cells | SRI |
| | | Ciliated Cells | | | |
| AZGP1 high Goblet Cells | FXYD3 | Early Response FOXJ1 high | SURF1 | FOXJ1 high Ciliated Cells | FAM227A |
| | | Ciliated Cells | | | |
| AZGP1 high Goblet Cells | TACSTD2 | Early Response FOXJ1 high | UXT | FOXJ1 high Ciliated Cells | TMEM131 |
| | | Ciliated Cells | | | |
| AZGP1 high Goblet Cells | DCXR | Early Response FOXJ1 high | VNN3 | FOXJ1 high Ciliated Cells | CFAP300 |
| | | Ciliated Cells | | | |
| AZGP1 high Goblet Cells | ADIRF | Early Response FOXJ1 high | TMEM67 | FOXJ1 high Ciliated Cells | SCGB2A1 |
| | | Ciliated Cells | | | |
| AZGP1 high Goblet Cells | OAS1 | Early Response FOXJ1 high | GRAMD2A | FOXJ1 high Ciliated Cells | EPB41L4B |
| | | Ciliated Cells | | | |
| AZGP1 high Goblet Cells | ARHGDIB | Early Response FOXJ1 high | NAT1 | FOXJ1 high Ciliated Cells | MOK |
| | | Ciliated Cells | | | |
| AZGP1 high Goblet Cells | TMED3 | Early Response FOXJ1 high | CLBA1 | FOXJ1 high Ciliated Cells | IQCA1 |
| | | Ciliated Cells | | | |
| AZGP1 high Goblet Cells | UPK1B | Early Response FOXJ1 high | COPA | FOXJ1 high Ciliated Cells | KLHDC9 |
| | | Ciliated Cells | | | |
| AZGP1 high Goblet Cells | GSTP1 | Early Response FOXJ1 high | TMEM173 | FOXJ1 high Ciliated Cells | APPL2 |
| | | Ciliated Cells | | | |
| AZGP1 high Goblet Cells | CSTA | Early Response FOXJ1 high | DCAF5 | FOXJ1 high Ciliated Cells | DPY30 |
| | | Ciliated Cells | | | |
| AZGP1 high Goblet Cells | AKR1C1 | Early Response FOXJ1 high | MEAF6 | FOXJ1 high Ciliated Cells | HNRNPF |
| | | Ciliated Cells | | | |
| AZGP1 high Goblet Cells | TMPRSS4 | Early Response FOXJ1 high | MAPK1 | FOXJ1 high Ciliated Cells | CCDC173 |
| | | Ciliated Cells | | | |
| AZGP1 high Goblet Cells | CREB3L2 | Early Response FOXJ1 high | RGCC | FOXJ1 high Ciliated Cells | P4HA2 |
| | | Ciliated Cells | | | |
| AZGP1 high Goblet Cells | HLA-C | Early Response FOXJ1 high | UBL3 | FOXJ1 high Ciliated Cells | B3GNT5 |
| | | Ciliated Cells | | | |
| AZGP1 high Goblet Cells | NR2F6 | Early Response FOXJ1 high | MLEC | FOXJ1 high Ciliated Cells | PLXNB1 |
| | | Ciliated Cells | | | |
| AZGP1 high Goblet Cells | TSTA3 | Early Response FOXJ1 high | SNX3 | FOXJ1 high Ciliated Cells | SPATA6 |
| | | Ciliated Cells | | | |
| AZGP1 high Goblet Cells | RPL18 | Early Response FOXJ1 high | SSBP2 | FOXJ1 high Ciliated Cells | KIF2A |
| | | Ciliated Cells | | | |
| AZGP1 high Goblet Cells | IFI27 | Early Response FOXJ1 high | LRRC34 | FOXJ1 high Ciliated Cells | ZNF474 |
| | | Ciliated Cells | | | |
| AZGP1 high Goblet Cells | SLC15A2 | Early Response FOXJ1 high | CEP97 | FOXJ1 high Ciliated Cells | PLAC8 |
| | | Ciliated Cells | | | |
| AZGP1 high Goblet Cells | TUBB | Early Response FOXJ1 high | VPS25 | FOXJ1 high Ciliated Cells | HSPBP1 |
| | | Ciliated Cells | | | |
| AZGP1 high Goblet Cells | SEL1L3 | Early Response FOXJ1 high | TTBK2 | FOXJ1 high Ciliated Cells | CCP110 |
| | | Ciliated Cells | | | |
| AZGP1 high Goblet Cells | AKR1C2 | Early Response FOXJ1 high | B9D1 | FOXJ1 high Ciliated Cells | CERKL |
| | | Ciliated Cells | | | |
| AZGP1 high Goblet Cells | TNFSF10 | Early Response FOXJ1 high | ACBD3- | FOXJ1 high Ciliated Cells | RSPH14 |
| | | Ciliated Cells | AS1 | | |
| AZGP1 high Goblet Cells | SSR4 | Early Response FOXJ1 high | CAMSAP1 | FOXJ1 high Ciliated Cells | ALDH9A1 |
| | | Ciliated Cells | | | |
| AZGP1 high Goblet Cells | MAL2 | Early Response FOXJ1 high | SYAP1 | FOXJ1 high Ciliated Cells | CYB5A |
| | | Ciliated Cells | | | |
| AZGP1 high Goblet Cells | GALNT7 | Early Response FOXJ1 high | FBXO36 | FOXJ1 high Ciliated Cells | GRAMD2A |
| | | Ciliated Cells | | | |
| AZGP1 high Goblet Cells | TRIP6 | Early Response FOXJ1 high | BAD | FOXJ1 high Ciliated Cells | TRAK1 |
| | | Ciliated Cells | | | |
| AZGP1 high Goblet Cells | VWA1 | Early Response FOXJ1 high | TMEM107 | FOXJ1 high Ciliated Cells | PLCH1 |
| | | Ciliated Cells | | | |
| AZGP1 high Goblet Cells | ASAH1 | Early Response FOXJ1 high | NRP2 | FOXJ1 high Ciliated Cells | SYAP1 |
| | | Ciliated Cells | | | |
| AZGP1 high Goblet Cells | SDC1 | Early Response FOXJ1 high | PXN | FOXJ1 high Ciliated Cells | UNC93B1 |
| | | Ciliated Cells | | | |
| AZGP1 high Goblet Cells | GALE | Early Response FOXJ1 high | PLA2G16 | FOXJ1 high Ciliated Cells | WDR13 |
| | | Ciliated Cells | | | |
| AZGP1 high Goblet Cells | RAB25 | Early Response FOXJ1 high | CTNNB1 | FOXJ1 high Ciliated Cells | LPAR3 |
| | | Ciliated Cells | | | |
| AZGP1 high Goblet Cells | TCEA3 | Early Response FOXJ1 high | POLD2 | FOXJ1 high Ciliated Cells | ZC2HC1A |
| | | Ciliated Cells | | | |
| AZGP1 high Goblet Cells | RHOC | Early Response FOXJ1 high | PDCD6IP | FOXJ1 high Ciliated Cells | SELENBP1 |
| | | Ciliated Cells | | | |
| AZGP1 high Goblet Cells | CAPN8 | Early Response FOXJ1 high | MUCL1 | FOXJ1 high Ciliated Cells | AC084033.3 |
| | | Ciliated Cells | | | |
| AZGP1 high Goblet Cells | CTSC | Early Response FOXJ1 high | IFFO2 | FOXJ1 high Ciliated Cells | CCT2 |
| | | Ciliated Cells | | | |
| AZGP1 high Goblet Cells | ERN2 | Early Response FOXJ1 high | COA3 | FOXJ1 high Ciliated Cells | CCDC60 |
| | | Ciliated Cells | | | |
| AZGP1 high Goblet Cells | ATP10B | Early Response FOXJ1 high | IFT88 | FOXJ1 high Ciliated Cells | GRIN3B |
| | | Ciliated Cells | | | |
| AZGP1 high Goblet Cells | ID1 | Early Response FOXJ1 high | SPATA17 | FOXJ1 high Ciliated Cells | ANKRD65 |
| | | Ciliated Cells | | | |
| AZGP1 high Goblet Cells | FAM129A | Early Response FOXJ1 high | NHLRC4 | FOXJ1 high Ciliated Cells | B9D2 |
| | | Ciliated Cells | | | |
| AZGP1 high Goblet Cells | LGALS3 | Early Response FOXJ1 high | ZBTB4 | FOXJ1 high Ciliated Cells | GIPR |
| | | Ciliated Cells | | | |
| AZGP1 high Goblet Cells | PROM2 | Early Response FOXJ1 high | SGMS2 | FOXJ1 high Ciliated Cells | CSPP1 |
| | | Ciliated Cells | | | |
| AZGP1 high Goblet Cells | FUT3 | Early Response FOXJ1 high | CDHR4 | FOXJ1 high Ciliated Cells | GSTP1 |
| | | Ciliated Cells | | | |
| AZGP1 high Goblet Cells | B4GALT5 | Early Response FOXJ1 high | TTC16 | FOXJ1 high Ciliated Cells | MYO1E |
| | | Ciliated Cells | | | |
| AZGP1 high Goblet Cells | HLA-DRB1 | Early Response FOXJ1 high | PGK1 | FOXJ1 high Ciliated Cells | ATP2B4 |
| | | Ciliated Cells | | | |
| AZGP1 high Goblet Cells | VAMP5 | Early Response FOXJ1 high | MKRN1 | FOXJ1 high Ciliated Cells | KIF3A |
| | | Ciliated Cells | | | |
| AZGP1 high Goblet Cells | CDK2AP2 | Early Response FOXJ1 high | GAS7 | FOXJ1 high Ciliated Cells | LRRC56 |
| | | Ciliated Cells | | | |
| AZGP1 high Goblet Cells | LMO4 | Early Response FOXJ1 high | SSRP1 | FOXJ1 high Ciliated Cells | SPAG16 |
| | | Ciliated Cells | | | |
| AZGP1 high Goblet Cells | TCIRG1 | Early Response FOXJ1 high | 10-Mar | FOXJ1 high Ciliated Cells | DIAPH2 |
| | | Ciliated Cells | | | |
| AZGP1 high Goblet Cells | SFN | Early Response FOXJ1 high | DYNC2H1 | FOXJ1 high Ciliated Cells | BUD23 |
| | | Ciliated Cells | | | |
| AZGP1 high Goblet Cells | ARRB2 | Early Response FOXJ1 high | FOXA1 | FOXJ1 high Ciliated Cells | CLSTN1 |
| | | Ciliated Cells | | | |
| AZGP1 high Goblet Cells | CAPN5 | Early Response FOXJ1 high | PPP1R36 | FOXJ1 high Ciliated Cells | ATP2C2 |
| | | Ciliated Cells | | | |
| AZGP1 high Goblet Cells | NDUFS7 | Early Response FOXJ1 high | DRC7 | FOXJ1 high Ciliated Cells | ANXA2 |
| | | Ciliated Cells | | | |
| AZGP1 high Goblet Cells | PADI1 | Early Response FOXJ1 high | PRPS1 | FOXJ1 high Ciliated Cells | MTSS1 |
| | | Ciliated Cells | | | |
| AZGP1 high Goblet Cells | TMEM160 | Early Response FOXJ1 high | TIMM10B | FOXJ1 high Ciliated Cells | ODF2 |
| | | Ciliated Cells | | | |
| AZGP1 high Goblet Cells | SLC44A2 | Early Response FOXJ1 high | CD164L2 | FOXJ1 high Ciliated Cells | APOBEC4 |
| | | Ciliated Cells | | | |
| AZGP1 high Goblet Cells | LGI1 | Early Response FOXJ1 high | GNB1 | FOXJ1 high Ciliated Cells | DNAAF3 |
| | | Ciliated Cells | | | |
| AZGP1 high Goblet Cells | NAPRT | Early Response FOXJ1 high | CNDP2 | FOXJ1 high Ciliated Cells | SLC25A4 |
| | | Ciliated Cells | | | |
| AZGP1 high Goblet Cells | DTX4 | Early Response FOXJ1 high | CRY2 | FOXJ1 high Ciliated Cells | C17orf97 |
| | | Ciliated Cells | | | |
| AZGP1 high Goblet Cells | GCNT3 | Early Response FOXJ1 high | CFAP65 | FOXJ1 high Ciliated Cells | TEKT4 |
| | | Ciliated Cells | | | |
| AZGP1 high Goblet Cells | ECH1 | Early Response FOXJ1 high | ZFP90 | FOXJ1 high Ciliated Cells | CFAP61 |
| | | Ciliated Cells | | | |
| AZGP1 high Goblet Cells | BAG1 | Early Response FOXJ1 high | MAATS1 | FOXJ1 high Ciliated Cells | ZBED5-AS1 |
| | | Ciliated Cells | | | |
| AZGP1 high Goblet Cells | APRT | Early Response FOXJ1 high | CLTA | FOXJ1 high Ciliated Cells | KIF6 |
| | | Ciliated Cells | | | |
| AZGP1 high Goblet Cells | ARPC5 | Early Response FOXJ1 high | TTC5 | FOXJ1 high Ciliated Cells | PGRMC1 |
| | | Ciliated Cells | | | |
| AZGP1 high Goblet Cells | FUT6 | Early Response FOXJ1 high | DUSP14 | FOXJ1 high Ciliated Cells | MPDZ |
| | | Ciliated Cells | | | |
| AZGP1 high Goblet Cells | CKAP4 | Early Response FOXJ1 high | RPL4 | FOXJ1 high Ciliated Cells | WDR49 |
| | | Ciliated Cells | | | |
| AZGP1 high Goblet Cells | MISP | Early Response FOXJ1 high | ENDOG | FOXJ1 high Ciliated Cells | CAPS2 |
| | | Ciliated Cells | | | |
| AZGP1 high Goblet Cells | PRKAR2B | Early Response FOXJ1 high | PEX6 | FOXJ1 high Ciliated Cells | PNMA1 |
| | | Ciliated Cells | | | |
| AZGP1 high Goblet Cells | VCL | Early Response FOXJ1 high | APOBEC4 | FOXJ1 high Ciliated Cells | CDH1 |
| | | Ciliated Cells | | | |
| AZGP1 high Goblet Cells | TSPAN1 | Early Response FOXJ1 high | LSM4 | FOXJ1 high Ciliated Cells | CCDC191 |
| | | Ciliated Cells | | | |
| AZGP1 high Goblet Cells | GLRX | Early Response FOXJ1 high | CDKN2AIP | FOXJ1 high Ciliated Cells | AKAP6 |
| | | Ciliated Cells | | | |
| AZGP1 high Goblet Cells | MLPH | Early Response FOXJ1 high | B3GNT7 | FOXJ1 high Ciliated Cells | LRRC49 |
| | | Ciliated Cells | | | |
| AZGP1 high Goblet Cells | XIST | Early Response FOXJ1 high | LINC01765 | FOXJ1 high Ciliated Cells | CTNNAL1 |
| | | Ciliated Cells | | | |
| AZGP1 high Goblet Cells | ZG16B | Early Response FOXJ1 high | IQCH | FOXJ1 high Ciliated Cells | CLBA1 |
| | | Ciliated Cells | | | |
| AZGP1 high Goblet Cells | TUBA1C | Early Response FOXJ1 high | WNK1 | FOXJ1 high Ciliated Cells | PDLIM4 |
| | | Ciliated Cells | | | |
| AZGP1 high Goblet Cells | C19orf33 | Early Response FOXJ1 high | ADIPOR1 | FOXJ1 high Ciliated Cells | TSGA10 |
| | | Ciliated Cells | | | |
| AZGP1 high Goblet Cells | PAM | Early Response FOXJ1 high | FOCAD | FOXJ1 high Ciliated Cells | DNAL4 |
| | | Ciliated Cells | | | |
| AZGP1 high Goblet Cells | PPA1 | Early Response FOXJ1 high | WDR63 | FOXJ1 high Ciliated Cells | 10-Mar |
| | | Ciliated Cells | | | |
| AZGP1 high Goblet Cells | BMP3 | Early Response FOXJ1 high | PPP4R1 | FOXJ1 high Ciliated Cells | SEC62 |
| | | Ciliated Cells | | | |
| AZGP1 high Goblet Cells | B3GNT3 | Early Response FOXJ1 high | CALM2 | FOXJ1 high Ciliated Cells | DUOX1 |
| | | Ciliated Cells | | | |
| AZGP1 high Goblet Cells | TFCP2L1 | Early Response FOXJ1 high | DNAH10 | FOXJ1 high Ciliated Cells | SMAP2 |
| | | Ciliated Cells | | | |
| AZGP1 high Goblet Cells | VILL | Early Response FOXJ1 high | IQSEC1 | FOXJ1 high Ciliated Cells | ANXA7 |
| | | Ciliated Cells | | | |
| AZGP1 high Goblet Cells | GMDS | Early Response FOXJ1 high | TMEM68 | FOXJ1 high Ciliated Cells | FAM47E |
| | | Ciliated Cells | | | |
| AZGP1 high Goblet Cells | BACE2 | Early Response FOXJ1 high | CYP27A1 | FOXJ1 high Ciliated Cells | EPCAM |
| | | Ciliated Cells | | | |
| AZGP1 high Goblet Cells | SLC12A2 | Early Response FOXJ1 high | SLC12A7 | FOXJ1 high Ciliated Cells | CFAP54 |
| | | Ciliated Cells | | | |
| AZGP1 high Goblet Cells | LDLR | Early Response FOXJ1 high | CHCHD6 | FOXJ1 high Ciliated Cells | EML1 |
| | | Ciliated Cells | | | |
| AZGP1 high Goblet Cells | SERINC2 | Early Response FOXJ1 high | PSMD2 | FOXJ1 high Ciliated Cells | GPX4 |
| | | Ciliated Cells | | | |
| AZGP1 high Goblet Cells | RPS12 | Early Response FOXJ1 high | VEZF1 | FOXJ1 high Ciliated Cells | IQUB |
| | | Ciliated Cells | | | |
| AZGP1 high Goblet Cells | SREBF1 | Early Response FOXJ1 high | SLC35A2 | FOXJ1 high Ciliated Cells | ALS2CR12 |
| | | Ciliated Cells | | | |
| AZGP1 high Goblet Cells | PDZK1IP1 | Early Response FOXJ1 high | HSPA4 | FOXJ1 high Ciliated Cells | PALMD |
| | | Ciliated Cells | | | |
| AZGP1 high Goblet Cells | GNAI1 | Early Response FOXJ1 high | C10orf95 | FOXJ1 high Ciliated Cells | CCDC181 |
| | | Ciliated Cells | | | |
| AZGP1 high Goblet Cells | FBLN1 | Early Response FOXJ1 high | 6-Mar | FOXJ1 high Ciliated Cells | KNDC1 |
| | | Ciliated Cells | | | |
| AZGP1 high Goblet Cells | SMAGP | Early Response FOXJ1 high | 2-Sep | FOXJ1 high Ciliated Cells | STMND1 |
| | | Ciliated Cells | | | |
| AZGP1 high Goblet Cells | MESP1 | Early Response FOXJ1 high | PBXIP1 | FOXJ1 high Ciliated Cells | RGS22 |
| | | Ciliated Cells | | | |
| AZGP1 high Goblet Cells | SLC35C1 | Early Response FOXJ1 high | PCYT1B | FOXJ1 high Ciliated Cells | C16orf71 |
| | | Ciliated Cells | | | |
| AZGP1 high Goblet Cells | GNE | Early Response FOXJ1 high | HAGH | FOXJ1 high Ciliated Cells | EFCAB2 |
| | | Ciliated Cells | | | |
| AZGP1 high Goblet Cells | SARSCoV2- | Early Response FOXJ1 high | KNDC1 | FOXJ1 high Ciliated Cells | DENND6B |
| | 3prime | Ciliated Cells | | | |
| AZGP1 high Goblet Cells | ALG3 | Early Response FOXJ1 high | GMPR | FOXJ1 high Ciliated Cells | TPGS2 |
| | | Ciliated Cells | | | |
| AZGP1 high Goblet Cells | SLC16A3 | Early Response FOXJ1 high | CEP126 | FOXJ1 high Ciliated Cells | TTLL10 |
| | | Ciliated Cells | | | |
| AZGP1 high Goblet Cells | FASN | Early Response FOXJ1 high | MIPEP | FOXJ1 high Ciliated Cells | FAM13A |
| | | Ciliated Cells | | | |
| AZGP1 high Goblet Cells | NPDC1 | Early Response FOXJ1 high | TOB2 | FOXJ1 high Ciliated Cells | TMEM173 |
| | | Ciliated Cells | | | |
| AZGP1 high Goblet Cells | LINC01133 | Early Response FOXJ1 high | RHPN2 | FOXJ1 high Ciliated Cells | TOGARAM1 |
| | | Ciliated Cells | | | |
| AZGP1 high Goblet Cells | FAM173A | Early Response FOXJ1 high | BAG6 | FOXJ1 high Ciliated Cells | IRX3 |
| | | Ciliated Cells | | | |
| AZGP1 high Goblet Cells | NME1 | Early Response FOXJ1 high | FSTL1 | FOXJ1 high Ciliated Cells | EMB |
| | | Ciliated Cells | | | |
| AZGP1 high Goblet Cells | CXCL16 | Early Response FOXJ1 high | DEGS2 | FOXJ1 high Ciliated Cells | CCDC157 |
| | | Ciliated Cells | | | |
| AZGP1 high Goblet Cells | SLC16A7 | Early Response FOXJ1 high | NDUFAF3 | FOXJ1 high Ciliated Cells | UBXN11 |
| | | Ciliated Cells | | | |
| AZGP1 high Goblet Cells | GDPD3 | Early Response FOXJ1 high | FLOT1 | FOXJ1 high Ciliated Cells | DNAJB13 |
| | | Ciliated Cells | | | |
| AZGP1 high Goblet Cells | ADI1 | Early Response FOXJ1 high | IMPAD1 | FOXJ1 high Ciliated Cells | TMEM14B |
| | | Ciliated Cells | | | |
| AZGP1 high Goblet Cells | MPZL2 | Early Response FOXJ1 high | PGM2L1 | FOXJ1 high Ciliated Cells | PPP1R7 |
| | | Ciliated Cells | | | |
| AZGP1 high Goblet Cells | RAB3D | Early Response FOXJ1 high | SLC39A6 | FOXJ1 high Ciliated Cells | GBP6 |
| | | Ciliated Cells | | | |
| AZGP1 high Goblet Cells | TPD52L1 | Early Response FOXJ1 high | MBP | FOXJ1 high Ciliated Cells | AKAP9 |
| | | Ciliated Cells | | | |
| AZGP1 high Goblet Cells | IFITM1 | Early Response FOXJ1 high | HACD3 | FOXJ1 high Ciliated Cells | PSMB4 |
| | | Ciliated Cells | | | |
| AZGP1 high Goblet Cells | MBOAT2 | Early Response FOXJ1 high | FIS1 | FOXJ1 high Ciliated Cells | ABI2 |
| | | Ciliated Cells | | | |
| AZGP1 high Goblet Cells | ZNF185 | Early Response FOXJ1 high | ZBTB10 | FOXJ1 high Ciliated Cells | WDR35 |
| | | Ciliated Cells | | | |
| AZGP1 high Goblet Cells | TRPM4 | Early Response FOXJ1 high | RBM24 | FOXJ1 high Ciliated Cells | TP53BP1 |
| | | Ciliated Cells | | | |
| AZGP1 high Goblet Cells | IRAK3 | Early Response FOXJ1 high | AGTRAP | FOXJ1 high Ciliated Cells | SMPD2 |
| | | Ciliated Cells | | | |
| AZGP1 high Goblet Cells | MZT2B | Early Response FOXJ1 high | ELOF1 | FOXJ1 high Ciliated Cells | TOMM34 |
| | | Ciliated Cells | | | |
| AZGP1 high Goblet Cells | HSD17B10 | Early Response FOXJ1 high | MYO1E | FOXJ1 high Ciliated Cells | NPHP1 |
| | | Ciliated Cells | | | |
| AZGP1 high Goblet Cells | ANPEP | Early Response FOXJ1 high | USP11 | FOXJ1 high Ciliated Cells | GFM2 |
| | | Ciliated Cells | | | |
| AZGP1 high Goblet Cells | LAPTM4B | Early Response FOXJ1 high | CFAP61 | FOXJ1 high Ciliated Cells | POU2AF1 |
| | | Ciliated Cells | | | |
| AZGP1 high Goblet Cells | ALDH3B2 | Early Response FOXJ1 high | ADGB | FOXJ1 high Ciliated Cells | B9D1 |
| | | Ciliated Cells | | | |
| AZGP1 high Goblet Cells | SLC4A4 | Early Response FOXJ1 high | CSDE1 | FOXJ1 high Ciliated Cells | JHY |
| | | Ciliated Cells | | | |
| AZGP1 high Goblet Cells | MAOA | Early Response FOXJ1 high | ZNF652 | FOXJ1 high Ciliated Cells | SUN1 |
| | | Ciliated Cells | | | |
| AZGP1 high Goblet Cells | CFH | Early Response FOXJ1 high | RPN1 | FOXJ1 high Ciliated Cells | SPAG1 |
| | | Ciliated Cells | | | |
| AZGP1 high Goblet Cells | BCL2L15 | Early Response FOXJ1 high | KLHDC10 | FOXJ1 high Ciliated Cells | CELSR1 |
| | | Ciliated Cells | | | |
| AZGP1 high Goblet Cells | GALNT5 | Early Response FOXJ1 high | AC125611.4 | FOXJ1 high Ciliated Cells | WDR31 |
| | | Ciliated Cells | | | |
| AZGP1 high Goblet Cells | CTDSPL | Early Response FOXJ1 high | ZNF440 | FOXJ1 high Ciliated Cells | DNAJC10 |
| | | Ciliated Cells | | | |
| AZGP1 high Goblet Cells | KCNK5 | Early Response FOXJ1 high | CLUAP1 | FOXJ1 high Ciliated Cells | EFCAB11 |
| | | Ciliated Cells | | | |
| AZGP1 high Goblet Cells | VSIR | Early Response FOXJ1 high | GTF2F1 | FOXJ1 high Ciliated Cells | SERPINB6 |
| | | Ciliated Cells | | | |
| AZGP1 high Goblet Cells | SH3BGRL3 | Early Response FOXJ1 high | ADSSL1 | FOXJ1 high Ciliated Cells | MAPRE3 |
| | | Ciliated Cells | | | |
| AZGP1 high Goblet Cells | TRIM7 | Early Response FOXJ1 high | TTC12 | FOXJ1 high Ciliated Cells | NEK1 |
| | | Ciliated Cells | | | |
| AZGP1 high Goblet Cells | MGST1 | Early Response FOXJ1 high | KLF4 | FOXJ1 high Ciliated Cells | USP2 |
| | | Ciliated Cells | | | |
| AZGP1 high Goblet Cells | KYNU | Early Response FOXJ1 high | GAS8 | FOXJ1 high Ciliated Cells | CCT5 |
| | | Ciliated Cells | | | |
| AZGP1 high Goblet Cells | NUDT8 | Early Response FOXJ1 high | TRAF3 | FOXJ1 high Ciliated Cells | FBXW9 |
| | | Ciliated Cells | | | |
| AZGP1 high Goblet Cells | RNF152 | Early Response FOXJ1 high | FIBP | FOXJ1 high Ciliated Cells | ARMH1 |
| | | Ciliated Cells | | | |
| AZGP1 high Goblet Cells | DHCR7 | Early Response FOXJ1 high | UBC | FOXJ1 high Ciliated Cells | 6-Mar |
| | | Ciliated Cells | | | |
| AZGP1 high Goblet Cells | MFSD4A | Early Response FOXJ1 high | MDM2 | FOXJ1 high Ciliated Cells | MIPEP |
| | | Ciliated Cells | | | |
| AZGP1 high Goblet Cells | GNPNAT1 | Early Response FOXJ1 high | LINC01513 | FOXJ1 high Ciliated Cells | CEP97 |
| | | Ciliated Cells | | | |
| AZGP1 high Goblet Cells | FAM83A | Early Response FOXJ1 high | KIAA2012 | FOXJ1 high Ciliated Cells | LRRC43 |
| | | Ciliated Cells | | | |
| AZGP1 high Goblet Cells | AC104126.1 | Early Response FOXJ1 high | FAXDC2 | FOXJ1 high Ciliated Cells | TEX9 |
| | | Ciliated Cells | | | |
| AZGP1 high Goblet Cells | ATP1B3 | Early Response FOXJ1 high | TGOLN2 | FOXJ1 high Ciliated Cells | ANKRD54 |
| | | Ciliated Cells | | | |
| AZGP1 high Goblet Cells | ZNHIT6 | Early Response FOXJ1 high | BTBD1 | FOXJ1 high Ciliated Cells | MORN1 |
| | | Ciliated Cells | | | |
| AZGP1 high Goblet Cells | PRSS8 | Early Response FOXJ1 high | PDIA4 | FOXJ1 high Ciliated Cells | ZNF440 |
| | | Ciliated Cells | | | |
| AZGP1 high Goblet Cells | EPS8L1 | Early Response FOXJ1 high | BBS9 | FOXJ1 high Ciliated Cells | CDC14A |
| | | Ciliated Cells | | | |
| AZGP1 high Goblet Cells | RPS18 | Early Response FOXJ1 high | MDM1 | FOXJ1 high Ciliated Cells | KDM1B |
| | | Ciliated Cells | | | |
| AZGP1 high Goblet Cells | AMN | Early Response FOXJ1 high | ZC2HC1C | FOXJ1 high Ciliated Cells | FOCAD |
| | | Ciliated Cells | | | |
| AZGP1 high Goblet Cells | HPGD | Early Response FOXJ1 high | GLYR1 | FOXJ1 high Ciliated Cells | IQCK |
| | | Ciliated Cells | | | |
| AZGP1 high Goblet Cells | JUP | Early Response FOXJ1 high | CCDC146 | FOXJ1 high Ciliated Cells | AKNA |
| | | Ciliated Cells | | | |
| AZGP1 high Goblet Cells | RAB27B | Early Response FOXJ1 high | ZC3H6 | FOXJ1 high Ciliated Cells | BCAS3 |
| | | Ciliated Cells | | | |
| AZGP1 high Goblet Cells | MMP1 | Early Response FOXJ1 high | SEC62 | FOXJ1 high Ciliated Cells | DCBLD2 |
| | | Ciliated Cells | | | |
| AZGP1 high Goblet Cells | FHL2 | Early Response FOXJ1 high | CTXN1 | FOXJ1 high Ciliated Cells | ALMS1 |
| | | Ciliated Cells | | | |
| AZGP1 high Goblet Cells | CLEC7A | Early Response FOXJ1 high | CCP110 | FOXJ1 high Ciliated Cells | MORF4L2 |
| | | Ciliated Cells | | | |
| AZGP1 high Goblet Cells | ECE1 | Early Response FOXJ1 high | IGF1R | FOXJ1 high Ciliated Cells | C10orf95 |
| | | Ciliated Cells | | | |
| AZGP1 high Goblet Cells | RPS28 | Early Response FOXJ1 high | PTBP3 | FOXJ1 high Ciliated Cells | NME9 |
| | | Ciliated Cells | | | |
| AZGP1 high Goblet Cells | TMSB4X | Early Response FOXJ1 high | SH3BGRL | FOXJ1 high Ciliated Cells | DALRD3 |
| | | Ciliated Cells | | | |
| AZGP1 high Goblet Cells | TPM4 | Early Response FOXJ1 high | ANKRD45 | FOXJ1 high Ciliated Cells | TSTD1 |
| | | Ciliated Cells | | | |
| AZGP1 high Goblet Cells | GPRC5A | Early Response FOXJ1 high | UEVLD | FOXJ1 high Ciliated Cells | KIAA0556 |
| | | Ciliated Cells | | | |
| AZGP1 high Goblet Cells | SPINT1 | Early Response FOXJ1 high | RIBC2 | FOXJ1 high Ciliated Cells | EFCAB6 |
| | | Ciliated Cells | | | |
| Basal Cells | KRT5 | Early Response FOXJ1 high | KIF6 | FOXJ1 high Ciliated Cells | FDXR |
| | | Ciliated Cells | | | |
| Basal Cells | KRT15 | Early Response FOXJ1 high | MMP24OS | FOXJ1 high Ciliated Cells | HHLA2 |
| | | Ciliated Cells | | | |
| Basal Cells | COL7A1 | Early Response FOXJ1 high | PPM1G | FOXJ1 high Ciliated Cells | CALM2 |
| | | Ciliated Cells | | | |
| Basal Cells | DST | Early Response FOXJ1 high | ATP2C2 | FOXJ1 high Ciliated Cells | CDC16 |
| | | Ciliated Cells | | | |
| Basal Cells | EGR1 | Early Response FOXJ1 high | GUK1 | FOXJ1 high Ciliated Cells | SCCPDH |
| | | Ciliated Cells | | | |
| Basal Cells | FOSB | Early Response FOXJ1 high | BCL2L1 | FOXJ1 high Ciliated Cells | TXNRD1 |
| | | Ciliated Cells | | | |
| Basal Cells | TP63 | Early Response FOXJ1 high | SMDT1 | FOXJ1 high Ciliated Cells | ABHD12B |
| | | Ciliated Cells | | | |
| Basal Cells | LAMB1 | Early Response FOXJ1 high | CTNNA1 | FOXJ1 high Ciliated Cells | MDM1 |
| | | Ciliated Cells | | | |
| Basal Cells | TNC | Early Response FOXJ1 high | RHBDD2 | FOXJ1 high Ciliated Cells | MSI2 |
| | | Ciliated Cells | | | |
| Basal Cells | FOS | Early Response FOXJ1 high | ZNF24 | FOXJ1 high Ciliated Cells | CTSH |
| | | Ciliated Cells | | | |
| Basal Cells | EGFR | Early Response FOXJ1 high | AKIRIN1 | FOXJ1 high Ciliated Cells | PPP1R36 |
| | | Ciliated Cells | | | |
| Basal Cells | FGFR3 | Early Response FOXJ1 high | BSG | FOXJ1 high Ciliated Cells | C9orf72 |
| | | Ciliated Cells | | | |
| Basal Cells | FAT2 | Early Response FOXJ1 high | DDR1 | FOXJ1 high Ciliated Cells | RIBC2 |
| | | Ciliated Cells | | | |
| Basal Cells | EPAS1 | Early Response FOXJ1 high | HSD17B8 | FOXJ1 high Ciliated Cells | C2orf73 |
| | | Ciliated Cells | | | |
| Basal Cells | FN1 | Early Response FOXJ1 high | LRRC61 | FOXJ1 high Ciliated Cells | SNRNP25 |
| | | Ciliated Cells | | | |
| Basal Cells | SERPINF1 | Early Response FOXJ1 high | AC084033.3 | FOXJ1 high Ciliated Cells | BRD3OS |
| | | Ciliated Cells | | | |
| Basal Cells | POSTN | Early Response FOXJ1 high | TRIM37 | FOXJ1 high Ciliated Cells | ZNF487 |
| | | Ciliated Cells | | | |
| Basal Cells | JUN | Early Response FOXJ1 high | SMPD3 | FOXJ1 high Ciliated Cells | FSD1L |
| | | Ciliated Cells | | | |
| Basal Cells | CA12 | Early Response FOXJ1 high | AC004832.1 | FOXJ1 high Ciliated Cells | PPP4R3B |
| | | Ciliated Cells | | | |
| Basal Cells | KRT17 | Early Response FOXJ1 high | CFAP47 | FOXJ1 high Ciliated Cells | PRR18 |
| | | Ciliated Cells | | | |
| Basal Cells | CD44 | Early Response FOXJ1 high | TMED10 | FOXJ1 high Ciliated Cells | CNTRL |
| | | Ciliated Cells | | | |
| Basal Cells | OBSCN | Early Response FOXJ1 high | PCYT2 | FOXJ1 high Ciliated Cells | SNTB1 |
| | | Ciliated Cells | | | |
| Basal Cells | HSPA1A | Early Response FOXJ1 high | KIAA0232 | FOXJ1 high Ciliated Cells | IARS2 |
| | | Ciliated Cells | | | |
| Basal Cells | S100A2 | Early Response FOXJ1 high | TXLNB | FOXJ1 high Ciliated Cells | PSMB5 |
| | | Ciliated Cells | | | |
| Basal Cells | FLNA | Early Response FOXJ1 high | C1orf43 | FOXJ1 high Ciliated Cells | UBAC1 |
| | | Ciliated Cells | | | |
| Basal Cells | SEMA5A | Early Response FOXJ1 high | CFAP69 | FOXJ1 high Ciliated Cells | DIXDC1 |
| | | Ciliated Cells | | | |
| Basal Cells | FMO2 | Early Response FOXJ1 high | ETNK1 | FOXJ1 high Ciliated Cells | MCAT |
| | | Ciliated Cells | | | |
| Basal Cells | SLC38A2 | Early Response FOXJ1 high | TCTEX1D1 | FOXJ1 high Ciliated Cells | GLIPR2 |
| | | Ciliated Cells | | | |
| Basal Cells | RPLP1 | Early Response FOXJ1 high | GMPPB | FOXJ1 high Ciliated Cells | PHTF1 |
| | | Ciliated Cells | | | |
| Basal Cells | PABPC1 | Early Response FOXJ1 high | EFEMP1 | FOXJ1 high Ciliated Cells | PRDX1 |
| | | Ciliated Cells | | | |
| Basal Cells | HSPA1B | Early Response FOXJ1 high | STAU1 | FOXJ1 high Ciliated Cells | PTGES3 |
| | | Ciliated Cells | | | |
| Basal Cells | ADAM28 | Early Response FOXJ1 high | MED29 | FOXJ1 high Ciliated Cells | CAP2 |
| | | Ciliated Cells | | | |
| Basal Cells | MKL2 | Early Response FOXJ1 high | OAZ2 | FOXJ1 high Ciliated Cells | PPP1R14C |
| | | Ciliated Cells | | | |
| Basal Cells | RPS3 | Early Response FOXJ1 high | NUP50-DT | FOXJ1 high Ciliated Cells | CCDC74B |
| | | Ciliated Cells | | | |
| Basal Cells | BCAM | Early Response FOXJ1 high | CARS | FOXJ1 high Ciliated Cells | TXLNB |
| | | Ciliated Cells | | | |
| Basal Cells | RASSF6 | Early Response FOXJ1 high | RNF20 | FOXJ1 high Ciliated Cells | PLEKHS1 |
| | | Ciliated Cells | | | |
| Basal Cells | CD81 | Early Response FOXJ1 high | HAX1 | FOXJ1 high Ciliated Cells | EBNA1BP2 |
| | | Ciliated Cells | | | |
| Basal Cells | LAMA5 | Early Response FOXJ1 high | SLC16A5 | FOXJ1 high Ciliated Cells | C4orf47 |
| | | Ciliated Cells | | | |
| Basal Cells | TSHZ2 | Early Response FOXJ1 high | CCDC28A | FOXJ1 high Ciliated Cells | HACD3 |
| | | Ciliated Cells | | | |
| Basal Cells | RPL3 | Early Response FOXJ1 high | ACSBG1 | FOXJ1 high Ciliated Cells | SLC12A7 |
| | | Ciliated Cells | | | |
| Basal Cells | ECE1 | Early Response FOXJ1 high | CNBP | FOXJ1 high Ciliated Cells | GALC |
| | | Ciliated Cells | | | |
| Basal Cells | ALDH3A2 | Early Response FOXJ1 high | DZIP1L | FOXJ1 high Ciliated Cells | SEC14L1 |
| | | Ciliated Cells | | | |
| Basal Cells | PIK3R1 | Early Response FOXJ1 high | TRIM44 | FOXJ1 high Ciliated Cells | TYMP |
| | | Ciliated Cells | | | |
| Basal Cells | RPL8 | Early Response FOXJ1 high | GNA14 | FOXJ1 high Ciliated Cells | TEX26 |
| | | Ciliated Cells | | | |
| Basal Cells | MT1X | Early Response FOXJ1 high | ADD1 | FOXJ1 high Ciliated Cells | TRMT1L |
| | | Ciliated Cells | | | |
| Basal Cells | RPL18 | Early Response FOXJ1 high | CFAP161 | FOXJ1 high Ciliated Cells | LMLN |
| | | Ciliated Cells | | | |
| Basal Cells | IL33 | Early Response FOXJ1 high | SLAIN2 | FOXJ1 high Ciliated Cells | PARVA |
| | | Ciliated Cells | | | |
| Basal Cells | RPL13 | Early Response FOXJ1 high | THRAP3 | FOXJ1 high Ciliated Cells | GNA11 |
| | | Ciliated Cells | | | |
| Basal Cells | PTPRZ1 | Early Response FOXJ1 high | TRIM56 | FOXJ1 high Ciliated Cells | EPPIN |
| | | Ciliated Cells | | | |
| Basal Cells | NOP53 | Early Response FOXJ1 high | FAM168B | FOXJ1 high Ciliated Cells | FAXDC2 |
| | | Ciliated Cells | | | |
| Basal Cells | ITGB4 | Early Response FOXJ1 high | TEKT4 | FOXJ1 high Ciliated Cells | CCDC30 |
| | | Ciliated Cells | | | |
| Basal Cells | LRP1 | Early Response FOXJ1 high | TMX4 | FOXJ1 high Ciliated Cells | DYNLRB1 |
| | | Ciliated Cells | | | |
| Basal Cells | SFN | Early Response FOXJ1 high | PSD3 | FOXJ1 high Ciliated Cells | CFAP20 |
| | | Ciliated Cells | | | |
| Basal Cells | RACK1 | Early Response FOXJ1 high | DAD1 | FOXJ1 high Ciliated Cells | GLT8D1 |
| | | Ciliated Cells | | | |
| Basal Cells | TXNIP | Early Response FOXJ1 high | CCDC89 | FOXJ1 high Ciliated Cells | CCDC89 |
| | | Ciliated Cells | | | |
| Basal Cells | RPS18 | Early Response FOXJ1 high | STAM2 | FOXJ1 high Ciliated Cells | C11orf49 |
| | | Ciliated Cells | | | |
| Basal Cells | RPS6 | Early Response FOXJ1 high | OTUD4 | FOXJ1 high Ciliated Cells | TMEM68 |
| | | Ciliated Cells | | | |
| Basal Cells | PRNP | Early Response FOXJ1 high | MTF1 | FOXJ1 high Ciliated Cells | CBY1 |
| | | Ciliated Cells | | | |
| Basal Cells | LMO4 | Early Response FOXJ1 high | AKNA | FOXJ1 high Ciliated Cells | XPNPEP3 |
| | | Ciliated Cells | | | |
| Basal Cells | JAG1 | Early Response FOXJ1 high | USP10 | FOXJ1 high Ciliated Cells | DUBR |
| | | Ciliated Cells | | | |
| Basal Cells | RPS4X | Early Response FOXJ1 high | DNAAF2 | FOXJ1 high Ciliated Cells | DNER |
| | | Ciliated Cells | | | |
| Basal Cells | RPS21 | Early Response FOXJ1 high | DAZAP2 | FOXJ1 high Ciliated Cells | HSBP1 |
| | | Ciliated Cells | | | |
| Basal Cells | ZFP36L1 | Early Response FOXJ1 high | SYS1 | FOXJ1 high Ciliated Cells | ZNF688 |
| | | Ciliated Cells | | | |
| Basal Cells | RPS16 | Early Response FOXJ1 high | SPAG1 | FOXJ1 high Ciliated Cells | PRDX3 |
| | | Ciliated Cells | | | |
| Basal Cells | RPL10A | Early Response FOXJ1 high | MORF4L2 | FOXJ1 high Ciliated Cells | ANKRD37 |
| | | Ciliated Cells | | | |
| Basal Cells | TPT1 | Early Response FOXJ1 high | PMM1 | FOXJ1 high Ciliated Cells | NAA20 |
| | | Ciliated Cells | | | |
| Basal Cells | TNS4 | Early Response FOXJ1 high | PPP6R1 | FOXJ1 high Ciliated Cells | TP73 |
| | | Ciliated Cells | | | |
| Basal Cells | RPL13A | Early Response FOXJ1 high | LRRC18 | FOXJ1 high Ciliated Cells | CCDC125 |
| | | Ciliated Cells | | | |
| Basal Cells | RPS12 | Early Response FOXJ1 high | MAP3K2 | FOXJ1 high Ciliated Cells | ANAPC5 |
| | | Ciliated Cells | | | |
| Basal Cells | RPS8 | Early Response FOXJ1 high | LINC02345 | FOXJ1 high Ciliated Cells | MEAF6 |
| | | Ciliated Cells | | | |
| Basal Cells | RPL4 | Early Response FOXJ1 high | RAB31 | FOXJ1 high Ciliated Cells | HSP90AB1 |
| | | Ciliated Cells | | | |
| Basal Cells | PLCH2 | Early Response FOXJ1 high | DNAJB13 | FOXJ1 high Ciliated Cells | ATP5F1A |
| | | Ciliated Cells | | | |
| Basal Cells | RPL7A | Early Response FOXJ1 high | SPCS1 | FOXJ1 high Ciliated Cells | ANKRD45 |
| | | Ciliated Cells | | | |
| Basal Cells | CLSTN1 | Early Response FOXJ1 high | MYO1B | FOXJ1 high Ciliated Cells | IFT74 |
| | | Ciliated Cells | | | |
| Basal Cells | RPL31 | Early Response FOXJ1 high | IQCK | FOXJ1 high Ciliated Cells | CIPC |
| | | Ciliated Cells | | | |
| Basal Cells | RPLP0 | Early Response FOXJ1 high | IRF6 | FOXJ1 high Ciliated Cells | KIFAP3 |
| | | Ciliated Cells | | | |
| Basal Cells | AQP3 | Early Response FOXJ1 high | ATP6AP2 | FOXJ1 high Ciliated Cells | SHANK2 |
| | | Ciliated Cells | | | |
| Basal Cells | SULF2 | Early Response FOXJ1 high | TSTD1 | FOXJ1 high Ciliated Cells | C10orf67 |
| | | Ciliated Cells | | | |
| Basal Cells | SLC25A6 | Early Response FOXJ1 high | ALDH1L1 | FOXJ1 high Ciliated Cells | NHLRC4 |
| | | Ciliated Cells | | | |
| Basal Cells | RPS23 | Early Response FOXJ1 high | KIF9 | FOXJ1 high Ciliated Cells | UGDH |
| | | Ciliated Cells | | | |
| Basal Cells | RPS19 | Early Response FOXJ1 high | RPS4Y1 | FOXJ1 high Ciliated Cells | INTU |
| | | Ciliated Cells | | | |
| Basal Cells | BOC | Early Response FOXJ1 high | SHANK2 | FOXJ1 high Ciliated Cells | XRN2 |
| | | Ciliated Cells | | | |
| Basal Cells | SULT1E1 | Early Response FOXJ1 high | CCNI | FOXJ1 high Ciliated Cells | RPGRIP1L |
| | | Ciliated Cells | | | |
| Basal Cells | RPL5 | Early Response FOXJ1 high | NAXE | FOXJ1 high Ciliated Cells | HSP90AA1 |
| | | Ciliated Cells | | | |
| Basal Cells | RPL11 | Early Response FOXJ1 high | SPAG7 | FOXJ1 high Ciliated Cells | DHX40 |
| | | Ciliated Cells | | | |
| Basal Cells | PDGFA | Early Response FOXJ1 high | EBLN3P | FOXJ1 high Ciliated Cells | AL163051.1 |
| | | Ciliated Cells | | | |
| Basal Cells | RPS5 | Early Response FOXJ1 high | TMEM254 | FOXJ1 high Ciliated Cells | RPGR |
| | | Ciliated Cells | | | |
| Basal Cells | NOTCH1 | Early Response FOXJ1 high | STK40 | FOXJ1 high Ciliated Cells | CTXN1 |
| | | Ciliated Cells | | | |
| Basal Cells | RPL30 | Early Response FOXJ1 high | WDR19 | FOXJ1 high Ciliated Cells | AMZ2 |
| | | Ciliated Cells | | | |
| Basal Cells | PCP4L1 | Early Response FOXJ1 high | CFAP77 | FOXJ1 high Ciliated Cells | TTC5 |
| | | Ciliated Cells | | | |
| Basal Cells | RPL19 | Early Response FOXJ1 high | BTBD9 | FOXJ1 high Ciliated Cells | WDR60 |
| | | Ciliated Cells | | | |
| Basal Cells | PKP1 | Early Response FOXJ1 high | ACTN1 | FOXJ1 high Ciliated Cells | ELN-AS1 |
| | | Ciliated Cells | | | |
| Basal Cells | RPS14 | Early Response FOXJ1 high | PTGES3 | FOXJ1 high Ciliated Cells | LPGAT1 |
| | | Ciliated Cells | | | |
| Basal Cells | DSC3 | Early Response FOXJ1 high | NUDT5 | FOXJ1 high Ciliated Cells | SSB |
| | | Ciliated Cells | | | |
| Basal Cells | LGR6 | Early Response FOXJ1 high | CFAP43 | FOXJ1 high Ciliated Cells | CFAP161 |
| | | Ciliated Cells | | | |
| Basal Cells | DKK3 | Early Response FOXJ1 high | WBP1L | FOXJ1 high Ciliated Cells | ZC2HC1C |
| | | Ciliated Cells | | | |
| BEST4 high Cilia high | CFAP157 | Early Response FOXJ1 high | C22orf15 | FOXJ1 high Ciliated Cells | ULK4 |
| Ciliated Cells | | Ciliated Cells | | | |
| BEST4 high Cilia high | DLEC1 | Early Response FOXJ1 high | TMED1 | FOXJ1 high Ciliated Cells | KIF27 |
| Ciliated Cells | | Ciliated Cells | | | |
| BEST4 high Cilia high | DNAAF1 | Early Response FOXJ1 high | PSMA5 | FOXJ1 high Ciliated Cells | CFAP77 |
| Ciliated Cells | | Ciliated Cells | | | |
| BEST4 high Cilia high | BEST4 | Early Response FOXJ1 high | STOML2 | FOXJ1 high Ciliated Cells | PPP1R42 |
| Ciliated Cells | | Ciliated Cells | | | |
| BEST4 high Cilia high | VWA3A | Early Response FOXJ1 high | CELSR1 | FOXJ1 high Ciliated Cells | CEP162 |
| Ciliated Cells | | Ciliated Cells | | | |
| BEST4 high Cilia high | TBC1D8 | Early Response FOXJ1 high | UBALD2 | FOXJ1 high Ciliated Cells | RABL2B |
| Ciliated Cells | | Ciliated Cells | | | |
| BEST4 high Cilia high | CROCC2 | Early Response FOXJ1 high | DYNLRB1 | FOXJ1 high Ciliated Cells | AK9 |
| Ciliated Cells | | Ciliated Cells | | | |
| BEST4 high Cilia high | CFAP100 | Early Response FOXJ1 high | H2AFV | FOXJ1 high Ciliated Cells | APOO |
| Ciliated Cells | | Ciliated Cells | | | |
| BEST4 high Cilia high | FRMPD2 | Early Response FOXJ1 high | SLC9A3R1 | FOXJ1 high Ciliated Cells | MAPK10 |
| Ciliated Cells | | Ciliated Cells | | | |
| BEST4 high Cilia high | RP1 | Early Response FOXJ1 high | G3BP2 | FOXJ1 high Ciliated Cells | DYNC2LI1 |
| Ciliated Cells | | Ciliated Cells | | | |
| BEST4 high Cilia high | CROCC | Early Response FOXJ1 high | TSPAN19 | FOXJ1 high Ciliated Cells | KIAA1671 |
| Ciliated Cells | | Ciliated Cells | | | |
| BEST4 high Cilia high | CFAP46 | Early Response FOXJ1 high | RBKS | FOXJ1 high Ciliated Cells | SULT1A1 |
| Ciliated Cells | | Ciliated Cells | | | |
| BEST4 high Cilia high | DNAH11 | Early Response FOXJ1 high | ANP32E | FOXJ1 high Ciliated Cells | CASC2 |
| Ciliated Cells | | Ciliated Cells | | | |
| BEST4 high Cilia high | CCDC17 | Early Response FOXJ1 high | ALCAM | FOXJ1 high Ciliated Cells | DDAH1 |
| Ciliated Cells | | Ciliated Cells | | | |
| BEST4 high Cilia high | DNAH3 | Early Response FOXJ1 high | ARF1 | FOXJ1 high Ciliated Cells | SPATA33 |
| Ciliated Cells | | Ciliated Cells | | | |
| BEST4 high Cilia high | DRC3 | Early Response FOXJ1 high | PITPNM1 | FOXJ1 high Ciliated Cells | STPG1 |
| Ciliated Cells | | Ciliated Cells | | | |
| BEST4 high Cilia high | CCDC114 | Early Response FOXJ1 high | GRAMD1C | FOXJ1 high Ciliated Cells | DZANK1 |
| Ciliated Cells | | Ciliated Cells | | | |
| BEST4 high Cilia high | MAPK15 | Early Response FOXJ1 high | UBE2Z | FOXJ1 high Ciliated Cells | ODF2L |
| Ciliated Cells | | Ciliated Cells | | | |
| BEST4 high Cilia high | CCDC187 | Early Response FOXJ1 high | C2orf73 | FOXJ1 high Ciliated Cells | CCDC13 |
| Ciliated Cells | | Ciliated Cells | | | |
| BEST4 high Cilia high | CFAP70 | Early Response FOXJ1 high | CYB5R1 | FOXJ1 high Ciliated Cells | LDHB |
| Ciliated Cells | | Ciliated Cells | | | |
| BEST4 high Cilia high | CDHR4 | Early Response FOXJ1 high | PIH1D2 | FOXJ1 high Ciliated Cells | LCA5L |
| Ciliated Cells | | Ciliated Cells | | | |
| BEST4 high Cilia high | LRRC74B | Early Response FOXJ1 high | NME9 | FOXJ1 high Ciliated Cells | DSTN |
| Ciliated Cells | | Ciliated Cells | | | |
| BEST4 high Cilia high | CCDC40 | Early Response FOXJ1 high | CHMP2A | FOXJ1 high Ciliated Cells | SEMA3C |
| Ciliated Cells | | Ciliated Cells | | | |
| BEST4 high Cilia high | DNAH1 | Early Response FOXJ1 high | DDX6 | FOXJ1 high Ciliated Cells | CRNDE |
| Ciliated Cells | | Ciliated Cells | | | |
| BEST4 high Cilia high | GIPR | Early Response FOXJ1 high | BEST4 | FOXJ1 high Ciliated Cells | MYB |
| Ciliated Cells | | Ciliated Cells | | | |
| BEST4 high Cilia high | DNAH6 | Early Response FOXJ1 high | ZNF688 | FOXJ1 high Ciliated Cells | TCTEX1D2 |
| Ciliated Cells | | Ciliated Cells | | | |
| BEST4 high Cilia high | DNAH7 | Early Response FOXJ1 high | IPO5 | FOXJ1 high Ciliated Cells | FAM104B |
| Ciliated Cells | | Ciliated Cells | | | |
| BEST4 high Cilia high | DNAH12 | Early Response FOXJ1 high | XPNPEP3 | FOXJ1 high Ciliated Cells | AC004832.1 |
| Ciliated Cells | | Ciliated Cells | | | |
| BEST4 high Cilia high | WDR90 | Early Response FOXJ1 high | NBR1 | FOXJ1 high Ciliated Cells | HSPH1 |
| Ciliated Cells | | Ciliated Cells | | | |
| BEST4 high Cilia high | EFHC1 | Early Response FOXJ1 high | XRCC5 | FOXJ1 high Ciliated Cells | PARK7 |
| Ciliated Cells | | Ciliated Cells | | | |
| BEST4 high Cilia high | CES4A | Early Response FOXJ1 high | MOK | FOXJ1 high Ciliated Cells | ANXA5 |
| Ciliated Cells | | Ciliated Cells | | | |
| BEST4 high Cilia high | DNAH10 | Early Response FOXJ1 high | AKR7A2 | FOXJ1 high Ciliated Cells | FAM161A |
| Ciliated Cells | | Ciliated Cells | | | |
| BEST4 high Cilia high | TTLL9 | Early Response FOXJ1 high | LRRC45 | FOXJ1 high Ciliated Cells | RPA3 |
| Ciliated Cells | | Ciliated Cells | | | |
| BEST4 high Cilia high | FAM227A | Early Response FOXJ1 high | CFAP58 | FOXJ1 high Ciliated Cells | MUC16 |
| Ciliated Cells | | Ciliated Cells | | | |
| BEST4 high Cilia high | PLXNB1 | Early Response FOXJ1 high | POMT2 | FOXJ1 high Ciliated Cells | MRPS31 |
| Ciliated Cells | | Ciliated Cells | | | |
| BEST4 high Cilia high | CFAP74 | Early Response FOXJ1 high | UBA1 | FOXJ1 high Ciliated Cells | MGST3 |
| Ciliated Cells | | Ciliated Cells | | | |
| BEST4 high Cilia high | CCDC189 | Early Response FOXJ1 high | FAM206A | FOXJ1 high Ciliated Cells | ANK3 |
| Ciliated Cells | | Ciliated Cells | | | |
| BEST4 high Cilia high | DZIP1L | Early Response FOXJ1 high | AES | FOXJ1 high Ciliated Cells | KCNRG |
| Ciliated Cells | | Ciliated Cells | | | |
| BEST4 high Cilia high | DNAI1 | Early Response FOXJ1 high | AL035661.1 | FOXJ1 high Ciliated Cells | AGR3 |
| Ciliated Cells | | Ciliated Cells | | | |
| BEST4 high Cilia high | SPEF2 | Early Response FOXJ1 high | HECTD1 | FOXJ1 high Ciliated Cells | HSPA4L |
| Ciliated Cells | | Ciliated Cells | | | |
| BEST4 high Cilia high | HAGHL | Early Response FOXJ1 high | C7orf50 | FOXJ1 high Ciliated Cells | TAX1BP1 |
| Ciliated Cells | | Ciliated Cells | | | |
| BEST4 high Cilia high | NEK10 | Early Response FOXJ1 high | DCTN3 | FOXJ1 high Ciliated Cells | SMIM22 |
| Ciliated Cells | | Ciliated Cells | | | |
| BEST4 high Cilia high | SYNE1 | Early Response FOXJ1 high | C7orf57 | FOXJ1 high Ciliated Cells | NLRP1 |
| Ciliated Cells | | Ciliated Cells | | | |
| BEST4 high Cilia high | CCDC57 | Early Response FOXJ1 high | RP1 | FOXJ1 high Ciliated Cells | EGLN3 |
| Ciliated Cells | | Ciliated Cells | | | |
| BEST4 high Cilia high | CFAP65 | Early Response FOXJ1 high | EID1 | FOXJ1 high Ciliated Cells | DMD |
| Ciliated Cells | | Ciliated Cells | | | |
| BEST4 high Cilia high | CFAP54 | Early Response FOXJ1 high | BPHL | FOXJ1 high Ciliated Cells | C5orf15 |
| Ciliated Cells | | Ciliated Cells | | | |
| BEST4 high Cilia high | DUOX1 | Early Response FOXJ1 high | YIPF6 | FOXJ1 high Ciliated Cells | FBXO36 |
| Ciliated Cells | | Ciliated Cells | | | |
| BEST4 high Cilia high | HYDIN | Early Response FOXJ1 high | GALM | FOXJ1 high Ciliated Cells | CPLANE1 |
| Ciliated Cells | | Ciliated Cells | | | |
| BEST4 high Cilia high | CDHR3 | Early Response FOXJ1 high | EIF4G3 | FOXJ1 high Ciliated Cells | PLCB4 |
| Ciliated Cells | | Ciliated Cells | | | |
| BEST4 high Cilia high | DNAH5 | Early Response FOXJ1 high | ERP29 | FOXJ1 high Ciliated Cells | C11orf74 |
| Ciliated Cells | | Ciliated Cells | | | |
| BEST4 high Cilia high | BAIAP3 | Early Response FOXJ1 high | CCDC125 | FOXJ1 high Ciliated Cells | HSPA8 |
| Ciliated Cells | | Ciliated Cells | | | |
| BEST4 high Cilia high | IFT172 | Early Response FOXJ1 high | FZD6 | FOXJ1 high Ciliated Cells | LINC02345 |
| Ciliated Cells | | Ciliated Cells | | | |
| BEST4 high Cilia high | TTC21A | Early Response FOXJ1 high | RILPL2 | FOXJ1 high Ciliated Cells | LINC01571 |
| Ciliated Cells | | Ciliated Cells | | | |
| BEST4 high Cilia high | SPPL2B | Early Response FOXJ1 high | LRRC27 | FOXJ1 high Ciliated Cells | NCALD |
| Ciliated Cells | | Ciliated Cells | | | |
| BEST4 high Cilia high | DNAH9 | Early Response FOXJ1 high | MRPL18 | FOXJ1 high Ciliated Cells | TFF3 |
| Ciliated Cells | | Ciliated Cells | | | |
| BEST4 high Cilia high | DCDC1 | Early Response FOXJ1 high | SH3RF3 | FOXJ1 high Ciliated Cells | NAP1L1 |
| Ciliated Cells | | Ciliated Cells | | | |
| BEST4 high Cilia high | ABCA13 | Early Response FOXJ1 high | C3orf67 | FOXJ1 high Ciliated Cells | CEP83 |
| Ciliated Cells | | Ciliated Cells | | | |
| BEST4 high Cilia high | KIAA2012 | Early Response FOXJ1 high | C6orf106 | FOXJ1 high Ciliated Cells | NBEA |
| Ciliated Cells | | Ciliated Cells | | | |
| BEST4 high Cilia high | MUC16 | Early Response FOXJ1 high | DLG3 | FOXJ1 high Ciliated Cells | GOLM1 |
| Ciliated Cells | | Ciliated Cells | | | |
| BEST4 high Cilia high | DNAH2 | Early Response FOXJ1 high | ARCN1 | FOXJ1 high Ciliated Cells | CD81 |
| Ciliated Cells | | Ciliated Cells | | | |
| BEST4 high Cilia high | RAD9A | Early Response FOXJ1 high | LDLRAD4 | FOXJ1 high Ciliated Cells | SYNE2 |
| Ciliated Cells | | Ciliated Cells | | | |
| BEST4 high Cilia high | EFCAB12 | Early Response FOXJ1 high | CISD3 | FOXJ1 high Ciliated Cells | HSPB11 |
| Ciliated Cells | | Ciliated Cells | | | |
| BEST4 high Cilia high | PRR29 | Early Response FOXJ1 high | ACTG1 | FOXJ1 high Ciliated Cells | YWHAE |
| Ciliated Cells | | Ciliated Cells | | | |
| BEST4 high Cilia high | ZBBX | Early Response FOXJ1 high | DNAI1 | FOXJ1 high Ciliated Cells | NME7 |
| Ciliated Cells | | Ciliated Cells | | | |
| BEST4 high Cilia high | MOK | Early Response FOXJ1 high | RMDN3 | FOXJ1 high Ciliated Cells | WARS |
| Ciliated Cells | | Ciliated Cells | | | |
| BEST4 high Cilia high | GABPB1-AS1 | Early Response FOXJ1 high | ARMC2 | FOXJ1 high Ciliated Cells | RPS4X |
| Ciliated Cells | | Ciliated Cells | | | |
| BEST4 high Cilia high | SLC25A36 | Early Response FOXJ1 high | INTS10 | HOPX high Squamous Cells | KRT13 |
| Ciliated Cells | | Ciliated Cells | | | |
| BEST4 high Cilia high | LRRIQ1 | Early Response FOXJ1 high | TMEM94 | HOPX high Squamous Cells | EMP1 |
| Ciliated Cells | | Ciliated Cells | | | |
| BEST4 high Cilia high | DTHD1 | Early Response FOXJ1 high | NEK4 | HOPX high Squamous Cells | MAL |
| Ciliated Cells | | Ciliated Cells | | | |
| BEST4 high Cilia high | CFAP69 | Early Response FOXJ1 high | IQANK1 | HOPX high Squamous Cells | HOPX |
| Ciliated Cells | | Ciliated Cells | | | |
| BEST4 high Cilia high | CFAP44 | Early Response FOXJ1 high | AP1M2 | HOPX high Squamous Cells | TMPRSS11B |
| Ciliated Cells | | Ciliated Cells | | | |
| BEST4 high Cilia high | SPAG17 | Early Response FOXJ1 high | ENSA | HOPX high Squamous Cells | KRT78 |
| Ciliated Cells | | Ciliated Cells | | | |
| BEST4 high Cilia high | TOGARAM2 | Early Response FOXJ1 high | COLCA1 | HOPX high Squamous Cells | CEACAM6 |
| Ciliated Cells | | Ciliated Cells | | | |
| BEST4 high Cilia high | DNHD1 | Early Response FOXJ1 high | ARL1 | HOPX high Squamous Cells | SCEL |
| Ciliated Cells | | Ciliated Cells | | | |
| BEST4 high Cilia high | SPAG8 | Early Response FOXJ1 high | CCT4 | HOPX high Squamous Cells | CEACAM5 |
| Ciliated Cells | | Ciliated Cells | | | |
| BEST4 high Cilia high | KIAA0556 | Early Response FOXJ1 high | RGL2 | HOPX high Squamous Cells | IL1RN |
| Ciliated Cells | | Ciliated Cells | | | |
| BEST4 high Cilia high | CCDC180 | Early Response FOXJ1 high | TEAD1 | HOPX high Squamous Cells | KRT23 |
| Ciliated Cells | | Ciliated Cells | | | |
| BEST4 high Cilia high | C11orf16 | Early Response FOXJ1 high | RRM2B | HOPX high Squamous Cells | RHCG |
| Ciliated Cells | | Ciliated Cells | | | |
| BEST4 high Cilia high | PPOX | Early Response FOXJ1 high | CCDC187 | HOPX high Squamous Cells | SPRR3 |
| Ciliated Cells | | Ciliated Cells | | | |
| BEST4 high Cilia high | FHAD1 | Early Response FOXJ1 high | ADSS | HOPX high Squamous Cells | CEACAM7 |
| Ciliated Cells | | Ciliated Cells | | | |
| BEST4 high Cilia high | CPLANE1 | Early Response FOXJ1 high | AL163051.1 | HOPX high Squamous Cells | PRSS27 |
| Ciliated Cells | | Ciliated Cells | | | |
| BEST4 high Cilia high | STK36 | Early Response FOXJ1 high | MID1 | HOPX high Squamous Cells | GPRC5A |
| Ciliated Cells | | Ciliated Cells | | | |
| BEST4 high Cilia high | IGSF10 | Early Response FOXJ1 high | EHF | HOPX high Squamous Cells | HSPB8 |
| Ciliated Cells | | Ciliated Cells | | | |
| BEST4 high Cilia high | UBXN11 | Early Response FOXJ1 high | CTBP2 | HOPX high Squamous Cells | C15orf48 |
| Ciliated Cells | | Ciliated Cells | | | |
| BEST4 high Cilia high | ZNF273 | Early Response FOXJ1 high | KIF3A | HOPX high Squamous Cells | CEACAM1 |
| Ciliated Cells | | Ciliated Cells | | | |
| BEST4 high Cilia high | TRMT9B | Early Response FOXJ1 high | CMBL | HOPX high Squamous Cells | ITGB8 |
| Ciliated Cells | | Ciliated Cells | | | |
| BEST4 high Cilia high | TSNAXIP1 | Early Response FOXJ1 high | TSPYL1 | HOPX high Squamous Cells | TP53INP2 |
| Ciliated Cells | | Ciliated Cells | | | |
| BEST4 high Cilia high | FANK1 | Early Response FOXJ1 high | ZNF33A | HOPX high Squamous Cells | TINAGL1 |
| Ciliated Cells | | Ciliated Cells | | | |
| BEST4 high Cilia high | TTLL10 | Early Response FOXJ1 high | KIAA0319L | HOPX high Squamous Cells | FAM83A |
| Ciliated Cells | | Ciliated Cells | | | |
| BEST4 high Cilia high | MAATS1 | Early Response FOXJ1 high | BBS2 | HOPX high Squamous Cells | LCN2 |
| Ciliated Cells | | Ciliated Cells | | | |
| BEST4 high Cilia high | DYNC2H1 | Early Response FOXJ1 high | TMEM50B | HOPX high Squamous Cells | PPL |
| Ciliated Cells | | Ciliated Cells | | | |
| BEST4 high Cilia high | CCDC78 | Early Response FOXJ1 high | ENO2 | HOPX high Squamous Cells | TMPRSS11E |
| Ciliated Cells | | Ciliated Cells | | | |
| BEST4 high Cilia high | POFUT2 | Early Response FOXJ1 high | PSMD1 | HOPX high Squamous Cells | A2ML1 |
| Ciliated Cells | | Ciliated Cells | | | |
| BEST4 high Cilia high | NEIL1 | Early Response FOXJ1 high | TP53INP1 | HOPX high Squamous Cells | S100A9 |
| Ciliated Cells | | Ciliated Cells | | | |
| BEST4 high Cilia high | TNNI3 | Early Response FOXJ1 high | NGRN | HOPX high Squamous Cells | ECM1 |
| Ciliated Cells | | Ciliated Cells | | | |
| BEST4 high Cilia high | ADPRHL2 | Early Response FOXJ1 high | SYPL1 | HOPX high Squamous Cells | GCNT3 |
| Ciliated Cells | | Ciliated Cells | | | |
| BEST4 high Cilia high | MAPK10 | Early Response FOXJ1 high | DNAI2 | HOPX high Squamous Cells | ALDH1A3 |
| Ciliated Cells | | Ciliated Cells | | | |
| BEST4 high Cilia high | ANKZF1 | Early Response FOXJ1 high | COG7 | HOPX high Squamous Cells | SERPINB2 |
| Ciliated Cells | | Ciliated Cells | | | |
| BEST4 high Cilia high | WDR66 | Early Response FOXJ1 high | RAB11A | HOPX high Squamous Cells | HIST1H2AC |
| Ciliated Cells | | Ciliated Cells | | | |
| BEST4 high Cilia high | NEAT1 | Early Response FOXJ1 high | TCTE1 | HOPX high Squamous Cells | TPM4 |
| Ciliated Cells | | Ciliated Cells | | | |
| BEST4 high Cilia high | CABIN1 | Early Response FOXJ1 high | HNRNPLL | HOPX high Squamous Cells | EPS8L1 |
| Ciliated Cells | | Ciliated Cells | | | |
| BEST4 high Cilia high | WDR49 | Early Response FOXJ1 high | TRAK2 | HOPX high Squamous Cells | NCCRP1 |
| Ciliated Cells | | Ciliated Cells | | | |
| BEST4 high Cilia high | CCDC190 | Early Response FOXJ1 high | TTLL5 | HOPX high Squamous Cells | CALML3 |
| Ciliated Cells | | Ciliated Cells | | | |
| BEST4 high Cilia high | TYMP | Early Response FOXJ1 high | DYRK2 | HOPX high Squamous Cells | RPTN |
| Ciliated Cells | | Ciliated Cells | | | |
| BEST4 high Cilia high | EXOC3 | Early Response FOXJ1 high | KCNRG | HOPX high Squamous Cells | FAM129B |
| Ciliated Cells | | Ciliated Cells | | | |
| BEST4 high Cilia high | C21orf58 | Early Response FOXJ1 high | PIGT | HOPX high Squamous Cells | HIST1H1C |
| Ciliated Cells | | Ciliated Cells | | | |
| BEST4 high Cilia high | CFAP43 | Early Response FOXJ1 high | LARP6 | HOPX high Squamous Cells | TMPRSS2 |
| Ciliated Cells | | Ciliated Cells | | | |
| BEST4 high Cilia high | EXOC7 | Early Response FOXJ1 high | IFT80 | HOPX high Squamous Cells | RAB11FIP1 |
| Ciliated Cells | | Ciliated Cells | | | |
| BEST4 high Cilia high | SLC25A29 | Early Response FOXJ1 high | COPS8 | HOPX high Squamous Cells | SPNS2 |
| Ciliated Cells | | Ciliated Cells | | | |
| BEST4 high Cilia high | CFAP99 | Early Response FOXJ1 high | FAM96B | HOPX high Squamous Cells | TTC9 |
| Ciliated Cells | | Ciliated Cells | | | |
| BEST4 high Cilia high | ZNF440 | Early Response FOXJ1 high | FAM47E | HOPX high Squamous Cells | TMBIM1 |
| Ciliated Cells | | Ciliated Cells | | | |
| BEST4 high Cilia high | CFAP73 | Early Response FOXJ1 high | NOA1 | HOPX high Squamous Cells | CAMK2N1 |
| Ciliated Cells | | Ciliated Cells | | | |
| BEST4 high Cilia high | ARMH1 | Early Response FOXJ1 high | GFOD2 | HOPX high Squamous Cells | XDH |
| Ciliated Cells | | Ciliated Cells | | | |
| BEST4 high Cilia high | LRRC71 | Early Response FOXJ1 high | KHDRBS1 | HOPX high Squamous Cells | KRT80 |
| Ciliated Cells | | Ciliated Cells | | | |
| BEST4 high Cilia high | TCTEX1D1 | Early Response FOXJ1 high | AMOT | HOPX high Squamous Cells | MXD1 |
| Ciliated Cells | | Ciliated Cells | | | |
| BEST4 high Cilia high | CFAP47 | Early Response FOXJ1 high | NDFIP2 | HOPX high Squamous Cells | LINC02303 |
| Ciliated Cells | | Ciliated Cells | | | |
| BEST4 high Cilia high | RFX3 | Early Response FOXJ1 high | PSMB3 | HOPX high Squamous Cells | TACSTD2 |
| Ciliated Cells | | Ciliated Cells | | | |
| BEST4 high Cilia high | ENOSF1 | Early Response FOXJ1 high | P4HA1 | HOPX high Squamous Cells | H1F0 |
| Ciliated Cells | | Ciliated Cells | | | |
| BEST4 high Cilia high | RBM20 | Early Response FOXJ1 high | CCDC151 | HOPX high Squamous Cells | SPINT1 |
| Ciliated Cells | | Ciliated Cells | | | |
| BEST4 high Cilia high | CEP126 | Early Response FOXJ1 high | WDR47 | HOPX high Squamous Cells | PIK3IP1 |
| Ciliated Cells | | Ciliated Cells | | | |
| BEST4 high Cilia high | ATP2C2 | Early Response FOXJ1 high | MBTPS1 | HOPX high Squamous Cells | PRSS8 |
| Ciliated Cells | | Ciliated Cells | | | |
| BEST4 high Cilia high | SPG7 | Early Response FOXJ1 high | TMEM219 | HOPX high Squamous Cells | TIMP2 |
| Ciliated Cells | | Ciliated Cells | | | |
| BEST4 high Cilia high | SFXN3 | Early Response FOXJ1 high | TOGARAM2 | HOPX high Squamous Cells | FUT3 |
| Ciliated Cells | | Ciliated Cells | | | |
| BEST4 high Cilia high | MYCBPAP | Early Response FOXJ1 high | AP3M2 | HOPX high Squamous Cells | NDRG2 |
| Ciliated Cells | | Ciliated Cells | | | |
| BEST4 high Cilia high | CCDC88C | Early Response FOXJ1 high | C10orf67 | HOPX high Squamous Cells | CTSB |
| Ciliated Cells | | Ciliated Cells | | | |
| BEST4 high Cilia high | TTLL3 | Early Response FOXJ1 high | PLPP5 | HOPX high Squamous Cells | RARRES3 |
| Ciliated Cells | | Ciliated Cells | | | |
| BEST4 high Cilia high | TTC16 | Early Response FOXJ1 high | HIST2H2BE | HOPX high Squamous Cells | MUC21 |
| Ciliated Cells | | Ciliated Cells | | | |
| BEST4 high Cilia high | ODF2L | Early Response FOXJ1 high | RABGAP1L | HOPX high Squamous Cells | PADI1 |
| Ciliated Cells | | Ciliated Cells | | | |
| BEST4 high Cilia high | CCDC30 | Early Response FOXJ1 high | ADD3 | HOPX high Squamous Cells | IVL |
| Ciliated Cells | | Ciliated Cells | | | |
| BEST4 high Cilia high | ADGB | Early Response FOXJ1 high | C21orf2 | HOPX high Squamous Cells | TMPRSS11D |
| Ciliated Cells | | Ciliated Cells | | | |
| BEST4 high Cilia high | RGL2 | Early Response FOXJ1 high | CFAP298 | HOPX high Squamous Cells | C1orf116 |
| Ciliated Cells | | Ciliated Cells | | | |
| BEST4 high Cilia high | ANKMY1 | Early Response FOXJ1 high | DYRK3 | HOPX high Squamous Cells | NECTIN4 |
| Ciliated Cells | | Ciliated Cells | | | |
| BEST4 high Cilia high | MTRNR2L1 | Early Response FOXJ1 high | RIPK4 | HOPX high Squamous Cells | FBXO32 |
| Ciliated Cells | | Ciliated Cells | | | |
| BEST4 high Cilia high | AK9 | Early Response FOXJ1 high | PYCR2 | HOPX high Squamous Cells | S100P |
| Ciliated Cells | | Ciliated Cells | | | |
| BEST4 high Cilia high | RABL2A | Early Response FOXJ1 high | PYCARD | HOPX high Squamous Cells | NT5C2 |
| Ciliated Cells | | Ciliated Cells | | | |
| BEST4 high Cilia high | IFT140 | Early Response FOXJ1 high | MGAT5 | HOPX high Squamous Cells | ABLIM3 |
| Ciliated Cells | | Ciliated Cells | | | |
| BEST4 high Cilia high | CRIP2 | Early Response FOXJ1 high | EFTUD2 | HOPX high Squamous Cells | EVPL |
| Ciliated Cells | | Ciliated Cells | | | |
| BEST4 high Cilia high | WDR27 | Early Response FOXJ1 high | C2orf50 | HOPX high Squamous Cells | KRT19 |
| Ciliated Cells | | Ciliated Cells | | | |
| BEST4 high Cilia high | IFT27 | Early Response FOXJ1 high | TMEM154 | HOPX high Squamous Cells | PPDPF |
| Ciliated Cells | | Ciliated Cells | | | |
| BEST4 high Cilia high | AKNA | Early Response FOXJ1 high | LDHB | HOPX high Squamous Cells | TMSB10 |
| Ciliated Cells | | Ciliated Cells | | | |
| BEST4 high Cilia high | ALMS1 | Early Response FOXJ1 high | BCL2L13 | HOPX high Squamous Cells | DUSP5 |
| Ciliated Cells | | Ciliated Cells | | | |
| BEST4 high Cilia high | TMEM63A | Early Response FOXJ1 high | LRP2BP | HOPX high Squamous Cells | ADGRF1 |
| Ciliated Cells | | Ciliated Cells | | | |
| BEST4 high Cilia high | ULK4 | Early Response FOXJ1 high | REPIN1 | HOPX high Squamous Cells | SPRR2A |
| Ciliated Cells | | Ciliated Cells | | | |
| BEST4 high Cilia high | TMEM67 | Early Response FOXJ1 high | KATNB1 | HOPX high Squamous Cells | ANXA11 |
| Ciliated Cells | | Ciliated Cells | | | |
| BEST4 high Cilia high | MAPK8IP3 | Early Response FOXJ1 high | TM9SF2 | HOPX high Squamous Cells | SQSTM1 |
| Ciliated Cells | | Ciliated Cells | | | |
| BEST4 high Cilia high | TEKT2 | Early Response FOXJ1 high | GBP3 | HOPX high Squamous Cells | MIR4435-2HG |
| Ciliated Cells | | Ciliated Cells | | | |
| BEST4 high Cilia high | ARHGAP39 | Early Response FOXJ1 high | VIM | HOPX high Squamous Cells | METRNL |
| Ciliated Cells | | Ciliated Cells | | | |
| BEST4 high Cilia high | HMGXB3 | Early Response FOXJ1 high | RPA3 | HOPX high Squamous Cells | SH3BGRL2 |
| Ciliated Cells | | Ciliated Cells | | | |
| BEST4 high Cilia high | INTS3 | Early Response FOXJ1 high | BNIP3L | HOPX high Squamous Cells | SNX18 |
| Ciliated Cells | | Ciliated Cells | | | |
| BEST4 high Cilia high | P4HTM | Early Response FOXJ1 high | C11orf74 | HOPX high Squamous Cells | PHLDA1 |
| Ciliated Cells | | Ciliated Cells | | | |
| BEST4 high Cilia high | MORN1 | Early Response FOXJ1 high | PARK7 | HOPX high Squamous Cells | BCAS1 |
| Ciliated Cells | | Ciliated Cells | | | |
| BEST4 high Cilia high | PASK | Early Response FOXJ1 high | TTC21A | HOPX high Squamous Cells | PDLIM5 |
| Ciliated Cells | | Ciliated Cells | | | |
| BEST4 high Cilia high | LENG8 | Early Response FOXJ1 high | ATPAF1 | HOPX high Squamous Cells | TMPRSS4 |
| Ciliated Cells | | Ciliated Cells | | | |
| BEST4 high Cilia high | RBM5 | Early Response FOXJ1 high | GALNT11 | HOPX high Squamous Cells | GALNT1 |
| Ciliated Cells | | Ciliated Cells | | | |
| BEST4 high Cilia high | INTU | Early Response FOXJ1 high | STYXL1 | HOPX high Squamous Cells | CDKN1A |
| Ciliated Cells | | Ciliated Cells | | | |
| BEST4 high Cilia high | KCNE1 | Early Response FOXJ1 high | RNF6 | HOPX high Squamous Cells | CDKN1B |
| Ciliated Cells | | Ciliated Cells | | | |
| BEST4 high Cilia high | DENND6B | Early Response FOXJ1 high | TSPYL4 | HOPX high Squamous Cells | SLC16A3 |
| Ciliated Cells | | Ciliated Cells | | | |
| BEST4 high Cilia high | DNAI2 | Early Response FOXJ1 high | DUBR | HOPX high Squamous Cells | ACTG1 |
| Ciliated Cells | | Ciliated Cells | | | |
| BEST4 high Cilia high | ODF3B | Early Response FOXJ1 high | DLAT | HOPX high Squamous Cells | FAM84A |
| Ciliated Cells | | Ciliated Cells | | | |
| BEST4 high Cilia high | SNED1 | Early Response FOXJ1 high | SF3B5 | HOPX high Squamous Cells | SLC6A14 |
| Ciliated Cells | | Ciliated Cells | | | |
| BEST4 high Cilia high | POMT2 | Early Response FOXJ1 high | POLR2E | HOPX high Squamous Cells | PITX1 |
| Ciliated Cells | | Ciliated Cells | | | |
| BEST4 high Cilia high | TSPOAP1 | Early Response FOXJ1 high | HERPUD1 | HOPX high Squamous Cells | KRT15 |
| Ciliated Cells | | Ciliated Cells | | | |
| BEST4 high Cilia high | ANKRD26 | Early Response FOXJ1 high | APLP2 | HOPX high Squamous Cells | FUT6 |
| Ciliated Cells | | Ciliated Cells | | | |
| BEST4 high Cilia high | ANKUB1 | Early Response FOXJ1 high | GLB1L2 | HOPX high Squamous Cells | CCNG2 |
| Ciliated Cells | | Ciliated Cells | | | |
| BEST4 high Cilia high | TTC6 | Early Response FOXJ1 high | TJP3 | HOPX high Squamous Cells | SGTA |
| Ciliated Cells | | Ciliated Cells | | | |
| BEST4 high Cilia high | WDR60 | Early Response FOXJ1 high | ACSS1 | HOPX high Squamous Cells | RIOK3 |
| Ciliated Cells | | Ciliated Cells | | | |
| BEST4 high Cilia high | MDH1B | Early Response FOXJ1 high | HADHA | HOPX high Squamous Cells | SDCBP2 |
| Ciliated Cells | | Ciliated Cells | | | |
| BEST4 high Cilia high | RABL2B | Early Response FOXJ1 high | LYRM2 | HOPX high Squamous Cells | IL36RN |
| Ciliated Cells | | Ciliated Cells | | | |
| BEST4 high Cilia high | STK11IP | Early Response FOXJ1 high | POU2AF1 | HOPX high Squamous Cells | C2orF54 |
| Ciliated Cells | | Ciliated Cells | | | |
| BEST4 high Cilia high | Cp164 | Early Response FOXJ1 high | KREMEN1 | HOPX high Squamous Cells | PTGES |
| Ciliated Cells | | Ciliated Cells | | | |
| BEST4 high Cilia high | AHI1 | Early Response FOXJ1 high | AL121899.1 | HOPX high Squamous Cells | MYL12A |
| Ciliated Cells | | Ciliated Cells | | | |
| BEST4 high Cilia high | NSUN7 | Early Response FOXJ1 high | PAF1 | HOPX high Squamous Cells | MBOAT2 |
| Ciliated Cells | | Ciliated Cells | | | |
| BEST4 high Cilia high | NEK5 | Early Response FOXJ1 high | DHX32 | HOPX high Squamous Cells | UBC |
| Ciliated Cells | | Ciliated Cells | | | |
| BEST4 high Cilia high | CCNL2 | Early Response FOXJ1 high | SPAG17 | HOPX high Squamous Cells | GABRP |
| Ciliated Cells | | Ciliated Cells | | | |
| BEST4 high Cilia high | CLMN | Early Response FOXJ1 high | UBAC1 | HOPX high Squamous Cells | CAP1 |
| Ciliated Cells | | Ciliated Cells | | | |
| BEST4 high Cilia high | ANKRD184 | Early Response FOXJ1 high | ABCD3 | HOPX high Squamous Cells | ANF117 |
| Ciliated Cells | | Ciliated Cells | | | |
| BEST4 high Cilia high | MUC4 | Early Response FOXJ1 high | CCT3 | HOPX high Squamous Cells | F3 |
| Ciliated Cells | | Ciliated Cells | | | |
| BEST4 high Cilia high | DNAJC10 | Early Response FOXJ1 high | NDUFS3 | HOPX high Squamous Cells | ISG15 |
| Ciliated Cells | | Ciliated Cells | | | |
| BEST4 high Cilia high | DCDC2B | Early Response FOXJ1 high | CCT6A | HOPX high Squamous Cells | EHD3 |
| Ciliated Cells | | Ciliated Cells | | | |
| BEST4 high Cilia high | C6orf118 | Early Response FOXJ1 high | SLC11A2 | HOPX high Squamous Cells | SPECC1 |
| Ciliated Cells | | Ciliated Cells | | | |
| BEST4 high Cilia high | AP006284.1 | Early Response FOXJ1 high | ATP6V0A4 | HOPX high Squamous Cells | STEAP4 |
| Ciliated Cells | | Ciliated Cells | | | |
| BEST4 high Cilia high | DDX17 | Early Response FOXJ1 high | NT5DC1 | HOPX high Squamous Cells | SLC31A2 |
| Ciliated Cells | | Ciliated Cells | | | |
| BEST4 high Cilia high | FER1L5 | Early Response FOXJ1 high | C5orf15 | HOPX high Squamous Cells | CDKN2B |
| Ciliated Cells | | Ciliated Cells | | | |
| BEST4 high Cilia high | IFT122 | Early Response FOXJ1 high | AC078864.2 | HOPX high Squamous Cells | DUSP4 |
| Ciliated Cells | | Ciliated Cells | | | |
| BEST4 high Cilia high | SPATA6L | Early Response FOXJ1 high | CLIC1 | HOPX high Squamous Cells | NET1 |
| Ciliated Cells | | Ciliated Cells | | | |
| BEST4 high Cilia high | SUN1 | Early Response FOXJ1 high | DCDC2 | HOPX high Squamous Cells | PABPC1 |
| Ciliated Cells | | Ciliated Cells | | | |
| BEST4 high Cilia high | DMD | Early Response FOXJ1 high | EEF2K | HOPX high Squamous Cells | KRT16 |
| Ciliated Cells | | Ciliated Cells | | | |
| BEST4 high Cilia high | IQUB | Early Response FOXJ1 high | TSGA10 | HOPX high Squamous Cells | PSCA |
| Ciliated Cells | | Ciliated Cells | | | |
| BEST4 high Cilia high | RBM6 | Early Response FOXJ1 high | NANS | HOPX high Squamous Cells | ZNF185 |
| Ciliated Cells | | Ciliated Cells | | | |
| BEST4 high Cilia high | MS4A8 | Early Response FOXJ1 high | MAK | HOPX high Squamous Cells | ERO1A |
| Ciliated Cells | | Ciliated Cells | | | |
| BEST4 high Cilia high | CCDC39 | Early Response FOXJ1 high | SDHA | HOPX high Squamous Cells | SASH1 |
| Ciliated Cells | | Ciliated Cells | | | |
| BEST4 high Cilia high | CSPP1 | Early Response FOXJ1 high | DHX57 | HOPX high Squamous Cells | MTUS1 |
| Ciliated Cells | | Ciliated Cells | | | |
| BEST4 high Cilia high | MRNIP | Early Response FOXJ1 high | CFAP100 | HOPX high Squamous Cells | FTH1 |
| Ciliated Cells | | Ciliated Cells | | | |
| BEST4 high Cilia high | MALAT1 | Early Response FOXJ1 high | SEPHS2 | HOPX high Squamous Cells | TUFT1 |
| Ciliated Cells | | Ciliated Cells | | | |
| BEST4 high Cilia high | DEFB124 | Early Response FOXJ1 high | SSU72 | HOPX high Squamous Cells | LITAF |
| Ciliated Cells | | Ciliated Cells | | | |
| BEST4 high Cilia high | GSTM2 | Early Response FOXJ1 high | NFIX | HOPX high Squamous Cells | TRIP10 |
| Ciliated Cells | | Ciliated Cells | | | |
| BEST4 high Cilia high | DDX5 | Early Response FOXJ1 high | KCTD1 | HOPX high Squamous Cells | PTK6 |
| Ciliated Cells | | Ciliated Cells | | | |
| BEST4 high Cilia high | TMEM190 | Early Response FOXJ1 high | ANAPC5 | HOPX high Squamous Cells | MAFF |
| Ciliated Cells | | Ciliated Cells | | | |
| BEST4 high Cilia high | CCDC146 | Early Response FOXJ1 high | RHOBTB1 | HOPX high Squamous Cells | VSIR |
| Ciliated Cells | | Ciliated Cells | | | |
| BEST4 high Cilia high | SYNE2 | Early Response FOXJ1 high | NUDT14 | HOPX high Squamous Cells | ERV3-1 |
| Ciliated Cells | | Ciliated Cells | | | |
| BEST4 high Cilia high | AKAP9 | Early Response FOXJ1 high | KCNH3 | HOPX high Squamous Cells | RND3 |
| Ciliated Cells | | Ciliated Cells | | | |
| BEST4 high Cilia high | RSPH1 | Early Response FOXJ1 high | KRT18 | HOPX high Squamous Cells | SLK |
| Ciliated Cells | | Ciliated Cells | | | |
| BEST4 high Cilia high | PIFO | Early Response FOXJ1 high | CACHD1 | HOPX high Squamous Cells | ASAH1 |
| Ciliated Cells | | Ciliated Cells | | | |
| BEST4 high Cilia high | TPPP3 | Early Response FOXJ1 high | B4GALT4 | HOPX high Squamous Cells | HIST1H2BD |
| Ciliated Cells | | Ciliated Cells | | | |
| BEST4 high Cilia high | CAPS | Early Response FOXJ1 high | EFCAB12 | HOPX high Squamous Cells | GALNT5 |
| Ciliated Cells | | Ciliated Cells | | | |
| BEST4 high Cilia high | MT-ND2 | Early Response FOXJ1 high | GPR107 | HOPX high Squamous Cells | SDC1 |
| Ciliated Cells | | Ciliated Cells | | | |
| BPIFA1 and Chemokine high | CXCL8 | Early Response FOXJ1 high | PTPRA | HOPX high Squamous Cells | DHRS3 |
| Secretory Cells | | Ciliated Cells | | | |
| BPIFA1 and Chemokine high | CXCL2 | Early Response FOXJ1 high | RUNDC1 | HOPX high Squamous Cells | SPINK5 |
| Secretory Cells | | Ciliated Cells | | | |
| BPIFA1 and Chemokine high | CXCL1 | Early Response FOXJ1 high | CAPS2 | HOPX high Squamous Cells | ST3GAL4 |
| Secretory Cells | | Ciliated Cells | | | |
| BPIFA1 and Chemokine high | CXCL3 | Early Response FOXJ1 high | TOMM20 | HOPX high Squamous Cells | GIPC1 |
| Secretory Cells | | Ciliated Cells | | | |
| BPIFA1 high Secretory Cells | MT-ND4L | Early Response FOXJ1 high | ZMYND12 | HOPX high Squamous Cells | USP6NL |
| | | Ciliated Cells | | | |
| BPIFA1 high Secretory Cells | CXCL8 | Early Response FOXJ1 high | USP9X | HOPX high Squamous Cells | SMAGP |
| | | Ciliated Cells | | | |
| BPIFA1 high Secretory Cells | BPIFA1 | Early Response FOXJ1 high | CA5B | HOPX high Squamous Cells | CST6 |
| | | Ciliated Cells | | | |
| BPIFA1 high Secretory Cells | MT-ND6 | Early Response FOXJ1 high | HSBP1 | HOPX high Squamous Cells | STK24 |
| | | Ciliated Cells | | | |
| BPIFA1 high Secretory Cells | MSMB | Early Response FOXJ1 high | RITA1 | HOPX high Squamous Cells | OPTN |
| | | Ciliated Cells | | | |
| BPIFA1 high Secretory Cells | CXCL1 | Early Response FOXJ1 high | TSNAXIP1 | HOPX high Squamous Cells | NAMPT |
| | | Ciliated Cells | | | |
| BPIFA1 high Secretory Cells | MT-ATP8 | Early Response FOXJ1 high | RAC1 | HOPX high Squamous Cells | LGALS3 |
| | | Ciliated Cells | | | |
| BPIFA1 high Secretory Cells | MT-ND3 | Early Response FOXJ1 high | IFT74 | HOPX high Squamous Cells | HIST1H4H |
| | | Ciliated Cells | | | |
| BPIFA1 high Secretory Cells | RPL41 | Early Response FOXJ1 high | AMPD3 | HOPX high Squamous Cells | B4GALT5 |
| | | Ciliated Cells | | | |
| BPIFA1 high Secretory Cells | MT-CO3 | Early Response FOXJ1 high | TUSC2 | HOPX high Squamous Cells | NAPRT |
| | | Ciliated Cells | | | |
| BPIFA1 high Secretory Cells | SERPINB3 | Early Response FOXJ1 high | EMC4 | HOPX high Squamous Cells | PLS3 |
| | | Ciliated Cells | | | |
| BPIFA1 high Secretory Cells | RPS27 | Early Response FOXJ1 high | CSNK1G1 | HOPX high Squamous Cells | ACTB |
| | | Ciliated Cells | | | |
| BPIFA1 high Secretory Cells | TMSB4X | Early Response FOXJ1 high | PDCD6 | HOPX high Squamous Cells | VSIG2 |
| | | Ciliated Cells | | | |
| BPIFA1 high Secretory Cells | MT-CO2 | Early Response FOXJ1 high | USP51 | HOPX high Squamous Cells | YPEL3 |
| | | Ciliated Cells | | | |
| BPIFA1 high Secretory Cells | RPS29 | Early Response FOXJ1 high | LRRC56 | HOPX high Squamous Cells | MYO5B |
| | | Ciliated Cells | | | |
| BPIFA1 high Secretory Cells | RPL34 | Early Response FOXJ1 high | WDR93 | HOPX high Squamous Cells | SRD5A3 |
| | | Ciliated Cells | | | |
| BPIFA1 high Secretory Cells | MT-ND1 | Early Response FOXJ1 high | HNRNPUL1 | HOPX high Squamous Cells | HIST3H2A |
| | | Ciliated Cells | | | |
| BPIFA1 high Secretory Cells | RPS18 | Early Response FOXJ1 high | NDUFS1 | HOPX high Squamous Cells | MYL6 |
| | | Ciliated Cells | | | |
| BPIFA1 high Secretory Cells | ATP5F1E | Early Response FOXJ1 high | ZNF33B | HOPX high Squamous Cells | TMEM127 |
| | | Ciliated Cells | | | |
| BPIFA1 high Secretory Cells | MT-CYB | Early Response FOXJ1 high | TOP2B | HOPX high Squamous Cells | MAL2 |
| | | Ciliated Cells | | | |
| BPIFA1 high Secretory Cells | MT-ND4 | Early Response FOXJ1 high | SPSB1 | HOPX high Squamous Cells | FAM3D |
| | | Ciliated Cells | | | |
| BPIFA1 high Secretory Cells | MT-ND5 | Early Response FOXJ1 high | RETREG1 | HOPX high Squamous Cells | IL18 |
| | | Ciliated Cells | | | |
| BPIFA1 high Secretory Cells | HNRNPK | Early Response FOXJ1 high | VAPB | HOPX high Squamous Cells | DUSP16 |
| | | Ciliated Cells | | | |
| CCL5 high Squamous Cells | MT-ND5 | Early Response FOXJ1 high | SLC30A9 | HOPX high Squamous Cells | KRT7 |
| | | Ciliated Cells | | | |
| CCL5 high Squamous Cells | CCL5 | Early Response FOXJ1 high | ALDH5A1 | HOPX high Squamous Cells | LMO7 |
| | | Ciliated Cells | | | |
| CCL5 high Squamous Cells | MT-ND4 | Early Response FOXJ1 high | RBM47 | HOPX high Squamous Cells | PRSS22 |
| | | Ciliated Cells | | | |
| CCL5 high Squamous Cells | S100A8 | Early Response FOXJ1 high | KIAA0895 | HOPX high Squamous Cells | AC019349.1 |
| | | Ciliated Cells | | | |
| CCL5 high Squamous Cells | SPRR3 | Early Response FOXJ1 high | MOB1A | HOPX high Squamous Cells | SAMD9 |
| | | Ciliated Cells | | | |
| CCL5 high Squamous Cells | S100A9 | Early Response FOXJ1 high | SESN1 | HOPX high Squamous Cells | LIPH |
| | | Ciliated Cells | | | |
| CCL5 high Squamous Cells | RPTN | Early Response FOXJ1 high | IDNK | HOPX high Squamous Cells | VPS4B |
| | | Ciliated Cells | | | |
| CCL5 high Squamous Cells | MT-CO1 | Early Response FOXJ1 high | STXBP4 | HOPX high Squamous Cells | ANXA1 |
| | | Ciliated Cells | | | |
| CCL5 high Squamous Cells | CRNN | Early Response FOXJ1 high | POLQ | HOPX high Squamous Cells | PHLDA3 |
| | | Ciliated Cells | | | |
| CCL5 high Squamous Cells | NR4A2 | Early Response FOXJ1 high | PRMT5 | HOPX high Squamous Cells | ZNRF1 |
| | | Ciliated Cells | | | |
| CCL5 high Squamous Cells | MT-CO2 | Early Response FOXJ1 high | CDCP1 | HOPX high Squamous Cells | C6orf132 |
| | | Ciliated Cells | | | |
| CCL5 high Squamous Cells | MAL | Early Response FOXJ1 high | PRICKLE2 | HOPX high Squamous Cells | YPEL5 |
| | | Ciliated Cells | | | |
| CCL5 high Squamous Cells | MT-ND3 | Early Response FOXJ1 high | ELOVL5 | HOPX high Squamous Cells | CYSRT1 |
| | | Ciliated Cells | | | |
| CCL5 high Squamous Cells | MT-CO3 | Early Response FOXJ1 high | CASC1 | HOPX high Squamous Cells | ARRB2 |
| | | Ciliated Cells | | | |
| CCL5 high Squamous Cells | MT-ND2 | Early Response FOXJ1 high | PDZD8 | HOPX high Squamous Cells | ATP10B |
| | | Ciliated Cells | | | |
| CCL5 high Squamous Cells | MT-CYB | Early Response FOXJ1 high | JTB | HOPX high Squamous Cells | SORT1 |
| | | Ciliated Cells | | | |
| CCL5 high Squamous Cells | MT-ND1 | Early Response FOXJ1 high | VPS28 | HOPX high Squamous Cells | SERINC2 |
| | | Ciliated Cells | | | |
| CCL5 high Squamous Cells | MT-ATP6 | Early Response FOXJ1 high | KIF24 | HOPX high Squamous Cells | NABP1 |
| | | Ciliated Cells | | | |
| CCL5 high Squamous Cells | CEACAM5 | Early Response FOXJ1 high | SGPL1 | HOPX high Squamous Cells | DOCK9 |
| | | Ciliated Cells | | | |
| CCL5 high Squamous Cells | MT-ATP8 | Early Response FOXJ1 high | SFMBT1 | HOPX high Squamous Cells | TRIM29 |
| | | Ciliated Cells | | | |
| CCL5 high Squamous Cells | KRT13 | Early Response FOXJ1 high | CELF1 | HOPX high Squamous Cells | ABHD5 |
| | | Ciliated Cells | | | |
| CCL5 high Squamous Cells | SLPI | Early Response FOXJ1 high | NUP50 | HOPX high Squamous Cells | TRIM16 |
| | | Ciliated Cells | | | |
| CCL5 high Squamous Cells | S100P | Early Response FOXJ1 high | FAM107B | HOPX high Squamous Cells | PPP1CB |
| | | Ciliated Cells | | | |
| CCL5 high Squamous Cells | NACA | Early Response FOXJ1 high | FAIM | HOPX high Squamous Cells | TACC1 |
| | | Ciliated Cells | | | |
| CCL5 high Squamous Cells | DNAJC7 | Early Response FOXJ1 high | TEX9 | HOPX high Squamous Cells | SH2D4A |
| | | Ciliated Cells | | | |
| CCL5 high Squamous Cells | PPP2CA | Early Response FOXJ1 high | TRMT9B | HOPX high Squamous Cells | SPRR1B |
| | | Ciliated Cells | | | |
| Cilia high Ciliated Cells | WDR60 | Early Response FOXJ1 high | C1QBP | HOPX high Squamous Cells | TJP1 |
| | | Ciliated Cells | | | |
| Cilia high Ciliated Cells | DMD | Early Response FOXJ1 high | SEC63 | HOPX high Squamous Cells | S100A16 |
| | | Ciliated Cells | | | |
| Cilia high Ciliated Cells | SYNE1 | Early Response FOXJ1 high | STAT3 | HOPX high Squamous Cells | YWHAZ |
| | | Ciliated Cells | | | |
| Cilia high Ciliated Cells | FHAD1 | Early Response FOXJ1 high | CCDC180 | HOPX high Squamous Cells | BAG1 |
| | | Ciliated Cells | | | |
| Cilia high Ciliated Cells | DNAH11 | Early Response FOXJ1 high | SLC4A8 | HOPX high Squamous Cells | ITPRIP |
| | | Ciliated Cells | | | |
| Cilia high Ciliated Cells | CSPP1 | Early Response FOXJ1 high | KIAA1522 | HOPX high Squamous Cells | CRYBG2 |
| | | Ciliated Cells | | | |
| Cilia high Ciliated Cells | ANKRD18A | Early Response FOXJ1 high | BCL2L2 | HOPX high Squamous Cells | HES4 |
| | | Ciliated Cells | | | |
| Cilia high Ciliated Cells | HYDIN | Early Response FOXJ1 high | PKP2 | HOPX high Squamous Cells | MYH14 |
| | | Ciliated Cells | | | |
| Cilia high Ciliated Cells | DNAH3 | Early Response FOXJ1 high | GRIN3B | HOPX high Squamous Cells | ADIPOR2 |
| | | Ciliated Cells | | | |
| Cilia high Ciliated Cells | CFAP44 | Early Response FOXJ1 high | DNAJA1 | HOPX high Squamous Cells | ANXA9 |
| | | Ciliated Cells | | | |
| Cilia high Ciliated Cells | SPEF2 | Early Response FOXJ1 high | ST8SIA4 | HOPX high Squamous Cells | EPS8L2 |
| | | Ciliated Cells | | | |
| Cilia high Ciliated Cells | KIAA2012 | Early Response FOXJ1 high | SHISA5 | HOPX high Squamous Cells | SAT1 |
| | | Ciliated Cells | | | |
| Cilia high Ciliated Cells | CFAP54 | Early Response FOXJ1 high | CAPRIN1 | HOPX high Squamous Cells | GRN |
| | | Ciliated Cells | | | |
| Cilia high Ciliated Cells | DNAH12 | Early Response FOXJ1 high | DZANK1 | HOPX high Squamous Cells | TMEM159 |
| | | Ciliated Cells | | | |
| Cilia high Ciliated Cells | SPAG17 | Early Response FOXJ1 high | LRRC43 | HOPX high Squamous Cells | STK39 |
| | | Ciliated Cells | | | |
| Cilia high Ciliated Cells | DNAH5 | Early Response FOXJ1 high | TRAPPC8 | HOPX high Squamous Cells | KAZN |
| | | Ciliated Cells | | | |
| Cilia high Ciliated Cells | CFAP43 | Early Response FOXJ1 high | UHMK1 | HOPX high Squamous Cells | S100A8 |
| | | Ciliated Cells | | | |
| Cilia high Ciliated Cells | AK9 | Early Response FOXJ1 high | CRNDE | HOPX high Squamous Cells | PTTG1IP |
| | | Ciliated Cells | | | |
| Cilia high Ciliated Cells | ZBBX | Early Response FOXJ1 high | DAG1 | HOPX high Squamous Cells | ITPKC |
| | | Ciliated Cells | | | |
| Cilia high Ciliated Cells | LRRIQ1 | Early Response FOXJ1 high | IPMK | HOPX high Squamous Cells | EPHA2 |
| | | Ciliated Cells | | | |
| Cilia high Ciliated Cells | DCDC1 | Early Response FOXJ1 high | PRPF8 | HOPX high Squamous Cells | MFSD4A |
| | | Ciliated Cells | | | |
| Cilia high Ciliated Cells | DNAH7 | Early Response FOXJ1 high | PCBP1 | HOPX high Squamous Cells | BCL2L11 |
| | | Ciliated Cells | | | |
| Cilia high Ciliated Cells | DNAH10 | Early Response FOXJ1 high | TNKS | HOPX high Squamous Cells | KIAA1551 |
| | | Ciliated Cells | | | |
| Cilia high Ciliated Cells | DNAH6 | Early Response FOXJ1 high | RAET1E | HOPX high Squamous Cells | ZFAND5 |
| | | Ciliated Cells | | | |
| Cilia high Ciliated Cells | DNAH9 | Early Response FOXJ1 high | SPAG16 | HOPX high Squamous Cells | B3GNT3 |
| | | Ciliated Cells | | | |
| Cilia high Ciliated Cells | ANKRD26 | Early Response FOXJ1 high | ADAR | HOPX high Squamous Cells | WDR1 |
| | | Ciliated Cells | | | |
| Cilia high Ciliated Cells | ANKRD18B | Early Response FOXJ1 high | PIN1 | HOPX high Squamous Cells | P4HB |
| | | Ciliated Cells | | | |
| Cilia high Ciliated Cells | CCDC30 | Early Response FOXJ1 high | ANKS1A | HOPX high Squamous Cells | PDP1 |
| | | Ciliated Cells | | | |
| Cilia high Ciliated Cells | DNAAF1 | Early Response FOXJ1 high | RPGR | HOPX high Squamous Cells | SPSB3 |
| | | Ciliated Cells | | | |
| Cilia high Ciliated Cells | CFAP46 | Early Response FOXJ1 high | GNAI2 | HOPX high Squamous Cells | LRP10 |
| | | Ciliated Cells | | | |
| Cilia high Ciliated Cells | DLEC1 | Early Response FOXJ1 high | WWC1 | HOPX high Squamous Cells | TMEM106B |
| | | Ciliated Cells | | | |
| Cilia high Ciliated Cells | MTRNR2L1 | Early Response FOXJ1 high | FYCO1 | HOPX high Squamous Cells | REEP3 |
| | | Ciliated Cells | | | |
| Cilia high Ciliated Cells | CCDC180 | Early Response FOXJ1 high | TMEM14C | HOPX high Squamous Cells | ASCC2 |
| | | Ciliated Cells | | | |
| Cilia high Ciliated Cells | CCDC39 | Early Response FOXJ1 high | AAED1 | HOPX high Squamous Cells | MUC20 |
| | | Ciliated Cells | | | |
| Cilia high Ciliated Cells | MNS1 | Early Response FOXJ1 high | ABHD10 | HOPX high Squamous Cells | OAS1 |
| | | Ciliated Cells | | | |
| Cilia high Ciliated Cells | MTRNR2L12 | Early Response FOXJ1 high | RPS6KA1 | HOPX high Squamous Cells | UBE2R2 |
| | | Ciliated Cells | | | |
| Cilia high Ciliated Cells | CFAP70 | Early Response FOXJ1 high | HIPK2 | HOPX high Squamous Cells | JUP |
| | | Ciliated Cells | | | |
| Cilia high Ciliated Cells | ADGB | Early Response FOXJ1 high | CEP41 | HOPX high Squamous Cells | CCNI |
| | | Ciliated Cells | | | |
| Cilia high Ciliated Cells | CCDC146 | Early Response FOXJ1 high | RBM43 | HOPX high Squamous Cells | ATP6V0E1 |
| | | Ciliated Cells | | | |
| Cilia high Ciliated Cells | SYNE2 | Early Response FOXJ1 high | TBCB | HOPX high Squamous Cells | TPD52L2 |
| | | Ciliated Cells | | | |
| Cilia high Ciliated Cells | VWA3A | Early Response FOXJ1 high | RTN4 | HOPX high Squamous Cells | PCBP1 |
| | | Ciliated Cells | | | |
| Cilia high Ciliated Cells | CEP128 | Early Response FOXJ1 high | ELP1 | HOPX high Squamous Cells | HIST1H2BG |
| | | Ciliated Cells | | | |
| Cilia high Ciliated Cells | CCDC40 | Early Response FOXJ1 high | JAK1 | HOPX high Squamous Cells | GPCPD1 |
| | | Ciliated Cells | | | |
| Cilia high Ciliated Cells | CASC1 | Early Response FOXJ1 high | STOM | HOPX high Squamous Cells | FOXC1 |
| | | Ciliated Cells | | | |
| Cilia high Ciliated Cells | MAATS1 | Early Response FOXJ1 high | DNAJA2 | HOPX high Squamous Cells | HEBP2 |
| | | Ciliated Cells | | | |
| Cilia high Ciliated Cells | CCDC191 | Early Response FOXJ1 high | NEK1 | HOPX high Squamous Cells | TMPRSS11A |
| | | Ciliated Cells | | | |
| Cilia high Ciliated Cells | RP1 | Early Response FOXJ1 high | IQCA1 | HOPX high Squamous Cells | RNASE7 |
| | | Ciliated Cells | | | |
| Cilia high Ciliated Cells | FRMPD2 | Early Response FOXJ1 high | PRPSAP1 | HOPX high Squamous Cells | BLVRB |
| | | Ciliated Cells | | | |
| Cilia high Ciliated Cells | CFAP74 | Early Response FOXJ1 high | NEDD4L | HOPX high Squamous Cells | VAT1 |
| | | Ciliated Cells | | | |
| Cilia high Ciliated Cells | WDR66 | Early Response FOXJ1 high | MTMR6 | HOPX high Squamous Cells | CARHSP1 |
| | | Ciliated Cells | | | |
| Cilia high Ciliated Cells | AHI1 | Early Response FOXJ1 high | NMRAL1 | HOPX high Squamous Cells | TRIP6 |
| | | Ciliated Cells | | | |
| Cilia high Ciliated Cells | CCDC173 | Early Response FOXJ1 high | MAGED1 | HOPX high Squamous Cells | ARHGDIB |
| | | Ciliated Cells | | | |
| Cilia high Ciliated Cells | ALS2CR12 | Early Response FOXJ1 high | PDHB | HOPX high Squamous Cells | ZFAND6 |
| | | Ciliated Cells | | | |
| Cilia high Ciliated Cells | ANKRD36 | Early Response FOXJ1 high | CCDC148 | HOPX high Squamous Cells | RNF10 |
| | | Ciliated Cells | | | |
| Cilia high Ciliated Cells | CFAP58 | Early Response FOXJ1 high | UBE2D3 | HOPX high Squamous Cells | TALDO1 |
| | | Ciliated Cells | | | |
| Cilia high Ciliated Cells | UTRN | Early Response FOXJ1 high | TCEA2 | HOPX high Squamous Cells | TNIP1 |
| | | Ciliated Cells | | | |
| Cilia high Ciliated Cells | FAM184A | Early Response FOXJ1 high | PUM2 | HOPX high Squamous Cells | RAC1 |
| | | Ciliated Cells | | | |
| Cilia high Ciliated Cells | DYNC2H1 | Early Response FOXJ1 high | PAIP2B | HOPX high Squamous Cells | RNPEPL1 |
| | | Ciliated Cells | | | |
| Cilia high Ciliated Cells | C6orf118 | Early Response FOXJ1 high | LMBRD1 | HOPX high Squamous Cells | MUC4 |
| | | Ciliated Cells | | | |
| Cilia high Ciliated Cells | CFAP57 | Early Response FOXJ1 high | ATF4 | HOPX high Squamous Cells | H3F3B |
| | | Ciliated Cells | | | |
| Cilia high Ciliated Cells | MT-ND2 | Early Response FOXJ1 high | ACYP1 | HOPX high Squamous Cells | PLAU |
| | | Ciliated Cells | | | |
| Cilia high Ciliated Cells | CEP126 | Early Response FOXJ1 high | WEE1 | HOPX high Squamous Cells | MYEOV |
| | | Ciliated Cells | | | |
| Cilia high Ciliated Cells | CLUAP1 | Early Response FOXJ1 high | NPHP1 | HOPX high Squamous Cells | KATNBL1 |
| | | Ciliated Cells | | | |
| Cilia high Ciliated Cells | TRAF3IP1 | Early Response FOXJ1 high | METRN | HOPX high Squamous Cells | ANKRD13A |
| | | Ciliated Cells | | | |
| Cilia high Ciliated Cells | ERICH3 | Early Response FOXJ1 high | KLF10 | HOPX high Squamous Cells | FCHO2 |
| | | Ciliated Cells | | | |
| Cilia high Ciliated Cells | MUC16 | Early Response FOXJ1 high | NRDC | HOPX high Squamous Cells | GDPD3 |
| | | Ciliated Cells | | | |
| Cilia high Ciliated Cells | CNTRL | Early Response FOXJ1 high | TXN2 | HOPX high Squamous Cells | B3GNT8 |
| | | Ciliated Cells | | | |
| Cilia high Ciliated Cells | DNAH1 | Early Response FOXJ1 high | EIF2D | HOPX high Squamous Cells | ATP6V1G1 |
| | | Ciliated Cells | | | |
| Cilia high Ciliated Cells | UPF3B | Early Response FOXJ1 high | RAB4A | HOPX high Squamous Cells | NDRG1 |
| | | Ciliated Cells | | | |
| Cilia high Ciliated Cells | CFAP100 | Early Response FOXJ1 high | ATXN7L1 | HOPX high Squamous Cells | TPT1 |
| | | Ciliated Cells | | | |
| Cilia high Ciliated Cells | WDR49 | Early Response FOXJ1 high | EFCAB6 | HOPX high Squamous Cells | CHP1 |
| | | Ciliated Cells | | | |
| Cilia high Ciliated Cells | DNAH2 | Early Response FOXJ1 high | TENT5C | HOPX high Squamous Cells | ADIPOR1 |
| | | Ciliated Cells | | | |
| Cilia high Ciliated Cells | DZIP1 | Early Response FOXJ1 high | NR2F2 | HOPX high Squamous Cells | APOL6 |
| | | Ciliated Cells | | | |
| Cilia high Ciliated Cells | CFAP45 | Early Response FOXJ1 high | IPP | HOPX high Squamous Cells | AL031777.3 |
| | | Ciliated Cells | | | |
| Cilia high Ciliated Cells | CEP83 | Early Response FOXJ1 high | AGPAT5 | HOPX high Squamous Cells | ADAM9 |
| | | Ciliated Cells | | | |
| Cilia high Ciliated Cells | DZIP1L | Early Response FOXJ1 high | TUBA1B | HOPX high Squamous Cells | CSNK1E |
| | | Ciliated Cells | | | |
| Cilia high Ciliated Cells | LUC7L3 | Early Response FOXJ1 high | RHPN1 | HOPX high Squamous Cells | ELF3 |
| | | Ciliated Cells | | | |
| Cilia high Ciliated Cells | IFT81 | Early Response FOXJ1 high | DPP7 | HOPX high Squamous Cells | KCNK6 |
| | | Ciliated Cells | | | |
| Cilia high Ciliated Cells | MT-ND1 | Early Response FOXJ1 high | TRIP12 | HOPX high Squamous Cells | HMGA1 |
| | | Ciliated Cells | | | |
| Cilia high Ciliated Cells | MT-CYB | Early Response FOXJ1 high | SAP30L | HOPX high Squamous Cells | RMND5A |
| | | Ciliated Cells | | | |
| Cilia high Ciliated Cells | ALMS1 | Early Response FOXJ1 high | KDM4A | HOPX high Squamous Cells | CAPN1 |
| | | Ciliated Cells | | | |
| Cilia high Ciliated Cells | IQUB | Early Response FOXJ1 high | NDUFAB1 | HOPX high Squamous Cells | TOM1 |
| | | Ciliated Cells | | | |
| Cilia high Ciliated Cells | IQCA1 | Early Response FOXJ1 high | GOLM1 | HOPX high Squamous Cells | VPS37B |
| | | Ciliated Cells | | | |
| Cilia high Ciliated Cells | CEP112 | Early Response FOXJ1 high | PARP1 | HOPX high Squamous Cells | SEC31A |
| | | Ciliated Cells | | | |
| Cilia high Ciliated Cells | CCDC88C | Early Response FOXJ1 high | CCDC74B | HOPX high Squamous Cells | CRB3 |
| | | Ciliated Cells | | | |
| Cilia high Ciliated Cells | NEK5 | Early Response FOXJ1 high | RGL1 | HOPX high Squamous Cells | B4GALT1 |
| | | Ciliated Cells | | | |
| Cilia high Ciliated Cells | CPLANE1 | Early Response FOXJ1 high | NDUFB10 | HOPX high Squamous Cells | NTN4 |
| | | Ciliated Cells | | | |
| Cilia high Ciliated Cells | FAM227A | Early Response FOXJ1 high | PEAK1 | HOPX high Squamous Cells | SLC12A6 |
| | | Ciliated Cells | | | |
| Cilia high Ciliated Cells | SREK1 | Early Response FOXJ1 high | DCAF6 | HOPX high Squamous Cells | RAB5IF |
| | | Ciliated Cells | | | |
| Cilia high Ciliated Cells | ULK4 | Early Response FOXJ1 high | ARMH4 | HOPX high Squamous Cells | PRDM1 |
| | | Ciliated Cells | | | |
| Cilia high Ciliated Cells | AKAP9 | Early Response FOXJ1 high | NFX1 | HOPX high Squamous Cells | PLSCR1 |
| | | Ciliated Cells | | | |
| Cilia high Ciliated Cells | INTU | Early Response FOXJ1 high | RAD23A | HOPX high Squamous Cells | LYPLA1 |
| | | Ciliated Cells | | | |
| Cilia high Ciliated Cells | CFAP157 | Early Response FOXJ1 high | TFCP2 | HOPX high Squamous Cells | RAB2A |
| | | Ciliated Cells | | | |
| Cilia high Ciliated Cells | ANKRD11 | Early Response FOXJ1 high | CD99 | HOPX high Squamous Cells | GNA15 |
| | | Ciliated Cells | | | |
| Cilia high Ciliated Cells | CROCC | Early Response FOXJ1 high | CDC14B | HOPX high Squamous Cells | PHACTR2 |
| | | Ciliated Cells | | | |
| Cilia high Ciliated Cells | DTHD1 | Early Response FOXJ1 high | CTSZ | HOPX high Squamous Cells | CYP2E1 |
| | | Ciliated Cells | | | |
| Cilia high Ciliated Cells | WDR63 | Early Response FOXJ1 high | SYNE1 | HOPX high Squamous Cells | UBAP1 |
| | | Ciliated Cells | | | |
| Cilia high Ciliated Cells | MACF1 | Early Response FOXJ1 high | EFCAB2 | HOPX high Squamous Cells | ARRDC3 |
| | | Ciliated Cells | | | |
| Cilia high Ciliated Cells | NBEA | Early Response FOXJ1 high | REEP5 | HOPX high Squamous Cells | PCDH1 |
| | | Ciliated Cells | | | |
| Cilia high Ciliated Cells | CLIP1 | Early Response FOXJ1 high | ZNF516 | HOPX high Squamous Cells | GRHL1 |
| | | Ciliated Cells | | | |
| Cilia high Ciliated Cells | PCM1 | Early Response FOXJ1 high | GHITM | HOPX high Squamous Cells | UBE2B |
| | | Ciliated Cells | | | |
| Cilia high Ciliated Cells | MT-ND3 | Early Response FOXJ1 high | LMAN2 | HOPX high Squamous Cells | ARL8B |
| | | Ciliated Cells | | | |
| Cilia high Ciliated Cells | GOLGB1 | Early Response FOXJ1 high | CES4A | HOPX high Squamous Cells | PICALM |
| | | Ciliated Cells | | | |
| Cilia high Ciliated Cells | ABCA13 | Early Response FOXJ1 high | PBX1 | HOPX high Squamous Cells | SPTSSA |
| | | Ciliated Cells | | | |
| Cilia high Ciliated Cells | MT-CO2 | Early Response FOXJ1 high | GDI1 | HOPX high Squamous Cells | MPRIP |
| | | Ciliated Cells | | | |
| Cilia high Ciliated Cells | MT-ND4 | Early Response FOXJ1 high | LARP1 | HOPX high Squamous Cells | LY6K |
| | | Ciliated Cells | | | |
| Cilia high Ciliated Cells | MT-ATP8 | Early Response FOXJ1 high | PTOV1 | HOPX high Squamous Cells | GABARAPL2 |
| | | Ciliated Cells | | | |
| Deuterosomal Cells | CDC20B | Early Response FOXJ1 high | UBXN6 | HOPX high Squamous Cells | KLK10 |
| | | Ciliated Cells | | | |
| Deuterosomal Cells | CCNO | Early Response FOXJ1 high | ASXL2 | HOPX high Squamous Cells | ATG9B |
| | | Ciliated Cells | | | |
| Deuterosomal Cells | HES6 | Early Response FOXJ1 high | MRPL41 | HOPX high Squamous Cells | UBL3 |
| | | Ciliated Cells | | | |
| Deuterosomal Cells | BTG3 | Early Response FOXJ1 high | EIF4B | HOPX high Squamous Cells | RAB5B |
| | | Ciliated Cells | | | |
| Deuterosomal Cells | FOXN4 | Early Response FOXJ1 high | PAPSS1 | HOPX high Squamous Cells | RER1 |
| | | Ciliated Cells | | | |
| Deuterosomal Cells | PLK4 | Early Response FOXJ1 high | PPP2R5C | HOPX high Squamous Cells | CSTA |
| | | Ciliated Cells | | | |
| Developing Ciliated Cells | MMACHC | Early Response FOXJ1 high | PRPF6 | HOPX high Squamous Cells | ITGB1 |
| | | Ciliated Cells | | | |
| Developing Ciliated Cells | BEST1 | Early Response FOXJ1 high | TERF2IP | HOPX high Squamous Cells | CPEB4 |
| | | Ciliated Cells | | | |
| Developing Ciliated Cells | FAM166A | Early Response FOXJ1 high | CPD | HOPX high Squamous Cells | RAB9A |
| | | Ciliated Cells | | | |
| Developing Ciliated Cells | SAA1 | Early Response FOXJ1 high | IQUB | HOPX high Squamous Cells | GABARAPL1 |
| | | Ciliated Cells | | | |
| Developing Ciliated Cells | AC093484.3 | Early Response FOXJ1 high | AKR1A1 | HOPX high Squamous Cells | CD99 |
| | | Ciliated Cells | | | |
| Developing Ciliated Cells | AGR3 | Early Response FOXJ1 high | SEC22C | HOPX high Squamous Cells | TCHP |
| | | Ciliated Cells | | | |
| Developing Ciliated Cells | MT-ND6 | Early Response FOXJ1 high | DHX30 | HOPX high Squamous Cells | HOOK3 |
| | | Ciliated Cells | | | |
| Developing Ciliated Cells | COX6A1 | Early Response FOXJ1 high | WDR45B | HOPX high Squamous Cells | ARPC5 |
| | | Ciliated Cells | | | |
| Developing Ciliated Cells | CALM2 | Early Response FOXJ1 high | JADE1 | HOPX high Squamous Cells | DUSP10 |
| | | Ciliated Cells | | | |
| Developing Ciliated Cells | RPL41 | Early Response FOXJ1 high | ATXN1 | HOPX high Squamous Cells | ZNF217 |
| | | Ciliated Cells | | | |
| Developing Ciliated Cells | PIFO | Early Response FOXJ1 high | POR | HOPX high Squamous Cells | SH3GL1 |
| | | Ciliated Cells | | | |
| Developing Ciliated Cells | UQCR11 | Early Response FOXJ1 high | SNX7 | HOPX high Squamous Cells | F11R |
| | | Ciliated Cells | | | |
| Developing Ciliated Cells | CAPSL | Early Response FOXJ1 high | PPP1R42 | HOPX high Squamous Cells | HIST2H2BE |
| | | Ciliated Cells | | | |
| Developing Ciliated Cells | FAM183A | Early Response FOXJ1 high | KDM1A | HOPX high Squamous Cells | FAM214A |
| | | Ciliated Cells | | | |
| Developing Ciliated Cells | TXN | Early Response FOXJ1 high | LRGUK | HOPX high Squamous Cells | CTSA |
| | | Ciliated Cells | | | |
| Developing Ciliated Cells | FTL | Early Response FOXJ1 high | CIRBP | HOPX high Squamous Cells | SP1 |
| | | Ciliated Cells | | | |
| Developing Ciliated Cells | ARL3 | Early Response FOXJ1 high | CEP104 | HOPX high Squamous Cells | S100A14 |
| | | Ciliated Cells | | | |
| Developing Ciliated Cells | AC020922.3 | Early Response FOXJ1 high | SORBS2 | HOPX high Squamous Cells | TMED3 |
| | | Ciliated Cells | | | |
| Developing Ciliated Cells | PRKAR1A | Early Response FOXJ1 high | DNAJC10 | HOPX high Squamous Cells | CNN2 |
| | | Ciliated Cells | | | |
| Developing Ciliated Cells | HSPB11 | Early Response FOXJ1 high | SOAT1 | HOPX high Squamous Cells | SAMD12 |
| | | Ciliated Cells | | | |
| Developing Ciliated Cells | ATP5F1E | Early Response FOXJ1 high | EIF3I | HOPX high Squamous Cells | TGFB1 |
| | | Ciliated Cells | | | |
| Developing Ciliated Cells | C11orf88 | Early Response FOXJ1 high | TTLL7 | HOPX high Squamous Cells | ARF6 |
| | | Ciliated Cells | | | |
| Developing Ciliated Cells | MLF1 | Early Response FOXJ1 high | CSPP1 | HOPX high Squamous Cells | MAP1LC3B |
| | | Ciliated Cells | | | |
| Developing Ciliated Cells | SCGB2A1 | Early Response FOXJ1 high | SERINC3 | HOPX high Squamous Cells | LASP1 |
| | | Ciliated Cells | | | |
| Developing Ciliated Cells | MT-ND4L | Early Response FOXJ1 high | GNG12 | HOPX high Squamous Cells | MARCKS |
| | | Ciliated Cells | | | |
| Developing Ciliated Cells | TSPAN19 | Early Response FOXJ1 high | KATNAL2 | HOPX high Squamous Cells | GPR87 |
| | | Ciliated Cells | | | |
| Developing Ciliated Cells | CFAP300 | Early Response FOXJ1 high | CHD4 | HOPX high Squamous Cells | SERINC1 |
| | | Ciliated Cells | | | |
| Developing Ciliated Cells | HSPE1 | Early Response FOXJ1 high | UQCRC1 | HOPX high Squamous Cells | NDFIP2 |
| | | Ciliated Cells | | | |
| Developing Ciliated Cells | TEX26 | Early Response FOXJ1 high | WDR49 | HOPX high Squamous Cells | NIPAL3 |
| | | Ciliated Cells | | | |
| Developing Ciliated Cells | AL357093.2 | Early Response FOXJ1 high | TMF1 | HOPX high Squamous Cells | SLC44A2 |
| | | Ciliated Cells | | | |
| Developing Ciliated Cells | NDUFB1 | Early Response FOXJ1 high | UPF1 | HOPX high Squamous Cells | RAB25 |
| | | Ciliated Cells | | | |
| Developing Ciliated Cells | YWHAE | Early Response FOXJ1 high | IRF2BP2 | HOPX high Squamous Cells | PAX9 |
| | | Ciliated Cells | | | |
| Developing Ciliated Cells | COX6C | Early Response FOXJ1 high | ARHGAP42 | HOPX high Squamous Cells | ARL8A |
| | | Ciliated Cells | | | |
| Developing Ciliated Cells | DYNLRB2 | Early Response FOXJ1 high | HINT2 | HOPX high Squamous Cells | GNG12 |
| | | Ciliated Cells | | | |
| Developing Ciliated Cells | NDUFA4 | Early Response FOXJ1 high | MAPRE3 | HOPX high Squamous Cells | CD68 |
| | | Ciliated Cells | | | |
| Developing Ciliated Cells | GON7 | Early Response FOXJ1 high | GALK2 | HOPX high Squamous Cells | CDCP1 |
| | | Ciliated Cells | | | |
| Developing Ciliated Cells | SKP1 | Early Response FOXJ1 high | MPP7 | HOPX high Squamous Cells | PEA15 |
| | | Ciliated Cells | | | |
| Developing Ciliated Cells | NME7 | Early Response FOXJ1 high | AGBL5 | HOPX high Squamous Cells | BRI3 |
| | | Ciliated Cells | | | |
| Developing Ciliated Cells | MORF4L1 | Early Response FOXJ1 high | PHGDH | HOPX high Squamous Cells | CLTB |
| | | Ciliated Cells | | | |
| Developing Ciliated Cells | SPAG16 | Early Response FOXJ1 high | SF3A1 | HOPX high Squamous Cells | TMSB4X |
| | | Ciliated Cells | | | |
| Developing Ciliated Cells | TCTEX1D2 | Early Response FOXJ1 high | DPM3 | HOPX high Squamous Cells | CORO2A |
| | | Ciliated Cells | | | |
| Developing Ciliated Cells | NME5 | Early Response FOXJ1 high | MVP | HOPX high Squamous Cells | RAB7A |
| | | Ciliated Cells | | | |
| Developing Ciliated Cells | SLIRP | Early Response FOXJ1 high | ADRB1 | HOPX high Squamous Cells | LEPROT |
| | | Ciliated Cells | | | |
| Developing Ciliated Cells | DPY30 | Early Response FOXJ1 high | CCT8 | HOPX high Squamous Cells | SDR16C5 |
| | | Ciliated Cells | | | |
| Developing Ciliated Cells | RPS27A | Early Response FOXJ1 high | MNS1 | HOPX high Squamous Cells | GNB2 |
| | | Ciliated Cells | | | |
| Developing Ciliated Cells | RPL34 | Early Response FOXJ1 high | HSPA4L | HOPX high Squamous Cells | EIF4EBP2 |
| | | Ciliated Cells | | | |
| Developing Ciliated Cells | DYDC2 | Early Response FOXJ1 high | VRK3 | HOPX high Squamous Cells | RNF141 |
| | | Ciliated Cells | | | |
| Developing Ciliated Cells | EFCAB10 | Early Response FOXJ1 high | ERCC1 | HOPX high Squamous Cells | HBP1 |
| | | Ciliated Cells | | | |
| Developing Ciliated Cells | PTGES3 | Early Response FOXJ1 high | PIAS3 | HOPX high Squamous Cells | KIFC3 |
| | | Ciliated Cells | | | |
| Developing Ciliated Cells | SAXO2 | Early Response FOXJ1 high | SATB1 | HOPX high Squamous Cells | DCAF12 |
| | | Ciliated Cells | | | |
| Developing Ciliated Cells | ARMC3 | Early Response FOXJ1 high | JKAMP | HOPX high Squamous Cells | SLC25A23 |
| | | Ciliated Cells | | | |
| Developing Ciliated Cells | NDUFA1 | Early Response FOXJ1 high | MED24 | HOPX high Squamous Cells | KIF1C |
| | | Ciliated Cells | | | |
| Developing Ciliated Cells | TAOK1 | Early Response FOXJ1 high | EEF2 | HOPX high Squamous Cells | FAM3C |
| | | Ciliated Cells | | | |
| Developing Ciliated Cells | PFN2 | Early Response FOXJ1 high | SCGB2A1 | HOPX high Squamous Cells | SBDS |
| | | Ciliated Cells | | | |
| Developing Ciliated Cells | SEM1 | Early Response FOXJ1 high | CMPK1 | HOPX high Squamous Cells | MROH6 |
| | | Ciliated Cells | | | |
| Developing Ciliated Cells | UGDH | Early Response FOXJ1 high | ARL6IP4 | HOPX high Squamous Cells | TRAF4 |
| | | Ciliated Cells | | | |
| Developing Ciliated Cells | PPIL6 | Early Response FOXJ1 high | RNFT2 | HOPX high Squamous Cells | RNF39 |
| | | Ciliated Cells | | | |
| Developing Ciliated Cells | COX7B | Early Response FOXJ1 high | SLC13A3 | HOPX high Squamous Cells | CLIC3 |
| | | Ciliated Cells | | | |
| Developing Ciliated Cells | PIH1D2 | Early Response FOXJ1 high | TMEM98 | HOPX high Squamous Cells | GOLPH3 |
| | | Ciliated Cells | | | |
| Developing Ciliated Cells | C9orf135 | Early Response FOXJ1 high | PPP5C | HOPX high Squamous Cells | WBP2 |
| | | Ciliated Cells | | | |
| Developing Ciliated Cells | DYNLT1 | Early Response FOXJ1 high | CFAP299 | HOPX high Squamous Cells | UBE2G1 |
| | | Ciliated Cells | | | |
| Developing Ciliated Cells | COX7C | Early Response FOXJ1 high | ACACA | HOPX high Squamous Cells | RALBP1 |
| | | Ciliated Cells | | | |
| Developing Ciliated Cells | TEX9 | Early Response FOXJ1 high | PRKCE | HOPX high Squamous Cells | CASP4 |
| | | Ciliated Cells | | | |
| Developing Ciliated Cells | CYSTM1 | Early Response FOXJ1 high | MDH2 | HOPX high Squamous Cells | CXCL17 |
| | | Ciliated Cells | | | |
| Developing Ciliated Cells | SRP14 | Early Response FOXJ1 high | PSMB8 | HOPX high Squamous Cells | CDC42SE1 |
| | | Ciliated Cells | | | |
| Developing Ciliated Cells | S100A6 | Early Response FOXJ1 high | YWHAB | HOPX high Squamous Cells | QSOX1 |
| | | Ciliated Cells | | | |
| Developing Ciliated Cells | C11orf74 | Early Response FOXJ1 high | TMEM14A | HOPX high Squamous Cells | AIF1L |
| | | Ciliated Cells | | | |
| Developing Ciliated Cells | CDC42 | Early Response FOXJ1 high | PKIB | HOPX high Squamous Cells | UBALD2 |
| | | Ciliated Cells | | | |
| Developing Ciliated Cells | MUC15 | Early Response FOXJ1 high | DNAJC16 | HOPX high Squamous Cells | HECA |
| | | Ciliated Cells | | | |
| Developing Ciliated Cells | ST13 | Early Response FOXJ1 high | COQ7 | HOPX high Squamous Cells | MAPK3 |
| | | Ciliated Cells | | | |
| Developing Ciliated Cells | CFAP298 | Early Response FOXJ1 high | MARVELD2 | HOPX high Squamous Cells | LMTK2 |
| | | Ciliated Cells | | | |
| Developing Ciliated Cells | PAIP2 | Early Response FOXJ1 high | IDE | HOPX high Squamous Cells | PTPRH |
| | | Ciliated Cells | | | |
| Developing Ciliated Cells | VAPA | Early Response FOXJ1 high | DUSP19 | HOPX high Squamous Cells | MTAP |
| | | Ciliated Cells | | | |
| Developing Ciliated Cells | HNRNPK | Early Response FOXJ1 high | PSMB1 | HOPX high Squamous Cells | TMEM50A |
| | | Ciliated Cells | | | |
| Developing Ciliated Cells | UQCR10 | Early Response FOXJ1 high | PTK2 | HOPX high Squamous Cells | PTPN12 |
| | | Ciliated Cells | | | |
| Developing Ciliated Cells | CALM1 | Early Response FOXJ1 high | PCSK5 | HOPX high Squamous Cells | ATP6V1E1 |
| | | Ciliated Cells | | | |
| Developing Ciliated Cells | SERF2 | Early Response FOXJ1 high | PZP | HOPX high Squamous Cells | MYO1C |
| | | Ciliated Cells | | | |
| Developing Ciliated Cells | LAP3 | Early Response FOXJ1 high | UBXN7 | HOPX high Squamous Cells | TRIM11 |
| | | Ciliated Cells | | | |
| Developing Ciliated Cells | UQCRQ | Early Response FOXJ1 high | YWHAH | HOPX high Squamous Cells | SNX9 |
| | | Ciliated Cells | | | |
| Developing Ciliated Cells | AZIN1 | Early Response FOXJ1 high | XPR1 | HOPX high Squamous Cells | MAP1LC3A |
| | | Ciliated Cells | | | |
| Developing Ciliated Cells | HSPH1 | Early Response FOXJ1 high | RCN2 | HOPX high Squamous Cells | REEP4 |
| | | Ciliated Cells | | | |
| Developing Ciliated Cells | ROMO1 | Early Response FOXJ1 high | PSPH | HOPX high Squamous Cells | MYD88 |
| | | Ciliated Cells | | | |
| Developing Ciliated Cells | NDUFB2 | Early Response FOXJ1 high | LPIN2 | HOPX high Squamous Cells | HIVEP2 |
| | | Ciliated Cells | | | |
| Developing Ciliated Cells | HSBP1 | Early Response FOXJ1 high | PCMTD2 | HOPX high Squamous Cells | RHBDL2 |
| | | Ciliated Cells | | | |
| Developing Ciliated Cells | DSTN | Early Response FOXJ1 high | CCDC88C | HOPX high Squamous Cells | VCL |
| | | Ciliated Cells | | | |
| Developing Ciliated Cells | ATP5MPL | Early Response FOXJ1 high | DDX42 | HOPX high Squamous Cells | RARG |
| | | Ciliated Cells | | | |
| Developing Ciliated Cells | ATP5ME | Early Response FOXJ1 high | PUF60 | HOPX high Squamous Cells | IL20RB |
| | | Ciliated Cells | | | |
| Developing Ciliated Cells | TSTD1 | Early Response FOXJ1 high | YWHAQ | HOPX high Squamous Cells | MGAT1 |
| | | Ciliated Cells | | | |
| Developing Ciliated Cells | GADD45GIP1 | Early Response FOXJ1 high | PELI1 | HOPX high Squamous Cells | ABTB2 |
| | | Ciliated Cells | | | |
| Developing Ciliated Cells | DBI | Early Response FOXJ1 high | KDM3B | HOPX high Squamous Cells | C9orf16 |
| | | Ciliated Cells | | | |
| Developing Ciliated Cells | SMIM22 | Early Response FOXJ1 high | C16orf45 | HOPX high Squamous Cells | CNP |
| | | Ciliated Cells | | | |
| Developing Ciliated Cells | NDUFC1 | Early Response FOXJ1 high | STX2 | HOPX high Squamous Cells | NKIRAS2 |
| | | Ciliated Cells | | | |
| Developing Ciliated Cells | SNX3 | Early Response FOXJ1 high | PSMD4 | HOPX high Squamous Cells | TOR1AIP2 |
| | | Ciliated Cells | | | |
| Developing Ciliated Cells | PRDX1 | Early Response FOXJ1 high | CCDC138 | HOPX high Squamous Cells | ARPC3 |
| | | Ciliated Cells | | | |
| Developing Ciliated Cells | HSP90AB1 | Early Response FOXJ1 high | RAB3IP | HOPX high Squamous Cells | STRN |
| | | Ciliated Cells | | | |
| Developing Ciliated Cells | MARCKS | Early Response FOXJ1 high | GRHL2 | HOPX high Squamous Cells | ABCA1 |
| | | Ciliated Cells | | | |
| Developing Ciliated Cells | NDUFA5 | Early Response FOXJ1 high | SIVA1 | HOPX high Squamous Cells | ZDHHC5 |
| | | Ciliated Cells | | | |
| Developing Ciliated Cells | CHMP5 | Early Response FOXJ1 high | PDPK1 | HOPX high Squamous Cells | DNAJC5 |
| | | Ciliated Cells | | | |
| Developing Ciliated Cells | SSB | Early Response FOXJ1 high | CLDN16 | HOPX high Squamous Cells | VGLL1 |
| | | Ciliated Cells | | | |
| Developing Ciliated Cells | MRNIP | Early Response FOXJ1 high | MAP4 | HOPX high Squamous Cells | PINK1 |
| | | Ciliated Cells | | | |
| Developing Ciliated Cells | DNAJA1 | Early Response FOXJ1 high | ELN-AS1 | HOPX high Squamous Cells | TAB2 |
| | | Ciliated Cells | | | |
| Developing Ciliated Cells | MORF4L2 | Early Response FOXJ1 high | ARMC9 | HOPX high Squamous Cells | CNPPD1 |
| | | Ciliated Cells | | | |
| Developing Ciliated Cells | HSPD1 | Early Response FOXJ1 high | DNAH7 | HOPX high Squamous Cells | SH3GLB1 |
| | | Ciliated Cells | | | |
| Developing Ciliated Cells | ATP5PF | Early Response FOXJ1 high | SMG7 | HOPX high Squamous Cells | MBD2 |
| | | Ciliated Cells | | | |
| Developing Ciliated Cells | TMED2 | Early Response FOXJ1 high | BTC | HOPX high Squamous Cells | RALA |
| | | Ciliated Cells | | | |
| Developing Ciliated Cells | CTSS | Early Response FOXJ1 high | CASTOR3 | HOPX high Squamous Cells | STN1 |
| | | Ciliated Cells | | | |
| Developing Ciliated Cells | TMBIM4 | Early Response FOXJ1 high | TMCO3 | HOPX high Squamous Cells | DIRC2 |
| | | Ciliated Cells | | | |
| Developing Ciliated Cells | PPP1CB | Early Response FOXJ1 high | FGGY | HOPX high Squamous Cells | ST14 |
| | | Ciliated Cells | | | |
| Developing Ciliated Cells | ERH | Early Response FOXJ1 high | CCL28 | HOPX high Squamous Cells | HM13 |
| | | Ciliated Cells | | | |
| Developing Ciliated Cells | MDM2 | Early Response FOXJ1 high | GLG1 | HOPX high Squamous Cells | C15orf62 |
| | | Ciliated Cells | | | |
| Developing Ciliated Cells | UFM1 | Early Response FOXJ1 high | ABCA13 | HOPX high Squamous Cells | ATP6V1B2 |
| | | Ciliated Cells | | | |
| Developing Ciliated Cells | NDUFB3 | Early Response FOXJ1 high | CRIM1 | HOPX high Squamous Cells | PRDM4 |
| | | Ciliated Cells | | | |
| Developing Ciliated Cells | SOD1 | Early Response FOXJ1 high | DDX24 | HOPX high Squamous Cells | GNB1 |
| | | Ciliated Cells | | | |
| Developing Ciliated Cells | RPA3 | Early Response FOXJ1 high | DHX9 | HOPX high Squamous Cells | ARNTL2 |
| | | Ciliated Cells | | | |
| Developing Ciliated Cells | CD164 | Early Response FOXJ1 high | AGPAT2 | HOPX high Squamous Cells | PAX6 |
| | | Ciliated Cells | | | |
| Developing Ciliated Cells | EEF1A1 | Early Response FOXJ1 high | ZNF3 | HOPX high Squamous Cells | HSPB1L1 |
| | | Ciliated Cells | | | |
| Developing Ciliated Cells | SRP9 | Early Response FOXJ1 high | CPNE3 | HOPX high Squamous Cells | RNF11 |
| | | Ciliated Cells | | | |
| Developing Ciliated Cells | PPT1 | Early Response FOXJ1 high | SAV1 | HOPX high Squamous Cells | CASP7 |
| | | Ciliated Cells | | | |
| Developing Ciliated Cells | SRI | Early Response FOXJ1 high | DDX1 | HOPX high Squamous Cells | NDFIP1 |
| | | Ciliated Cells | | | |
| Developing Ciliated Cells | CMTM6 | Early Response FOXJ1 high | UBE3D | HOPX high Squamous Cells | CHMP4C |
| | | Ciliated Cells | | | |
| Developing Ciliated Cells | HSPA8 | Early Response FOXJ1 high | PPP1CB | HOPX high Squamous Cells | PROM2 |
| | | Ciliated Cells | | | |
| Developing Ciliated Cells | KRT8 | Early Response FOXJ1 high | CNOT1 | HOPX high Squamous Cells | LYPD2 |
| | | Ciliated Cells | | | |
| Developing Ciliated Cells | COX7A2 | Early Response FOXJ1 high | MTCH2 | HOPX high Squamous Cells | C19orf33 |
| | | Ciliated Cells | | | |
| Developing Ciliated Cells | ADH7 | Early Response FOXJ1 high | LINC01571 | HOPX high Squamous Cells | PRAG1 |
| | | Ciliated Cells | | | |
| Developing Ciliated Cells | HMGN3 | Early Response FOXJ1 high | BECN1 | HOPX high Squamous Cells | RAB27B |
| | | Ciliated Cells | | | |
| Developing Ciliated Cells | MGST3 | Early Response FOXJ1 high | VAPA | HOPX high Squamous Cells | EPHB3 |
| | | Ciliated Cells | | | |
| Developing Ciliated Cells | B2M | Early Response FOXJ1 high | ETF1 | HOPX high Squamous Cells | POLB |
| | | Ciliated Cells | | | |
| Developing Ciliated Cells | C5orf15 | Early Response FOXJ1 high | HLTF | HOPX high Squamous Cells | MKNK2 |
| | | Ciliated Cells | | | |
| Developing Ciliated Cells | ADSS | Early Response FOXJ1 high | KCNE3 | HOPX high Squamous Cells | KLHL24 |
| | | Ciliated Cells | | | |
| Developing Ciliated Cells | ZMPSTE24 | Early Response FOXJ1 high | TCP11L2 | HOPX high Squamous Cells | SH3BGRL3 |
| | | Ciliated Cells | | | |
| Developing Ciliated Cells | ANXA1 | Early Response FOXJ1 high | GRSF1 | HOPX high Squamous Cells | VWA1 |
| | | Ciliated Cells | | | |
| Developing Ciliated Cells | NDUFB5 | Early Response FOXJ1 high | HSD17B12 | HOPX high Squamous Cells | SMIM5 |
| | | Ciliated Cells | | | |
| Developing Ciliated Cells | TMED10 | Early Response FOXJ1 high | HCLS1 | HOPX high Squamous Cells | CTTNBP2NL |
| | | Ciliated Cells | | | |
| Developing Ciliated Cells | MPC1 | Early Response FOXJ1 high | IFT52 | HOPX high Squamous Cells | KAT2B |
| | | Ciliated Cells | | | |
| Developing Ciliated Cells | CD9 | Early Response FOXJ1 high | CCDC114 | HOPX high Squamous Cells | CTSV |
| | | Ciliated Cells | | | |
| Developing Ciliated Cells | CLIC1 | Early Response FOXJ1 high | WDR11 | HOPX high Squamous Cells | NPC1 |
| | | Ciliated Cells | | | |
| Developing Ciliated Cells | TMEM123 | Early Response FOXJ1 high | GRB2 | HOPX high Squamous Cells | LGALS9 |
| | | Ciliated Cells | | | |
| Developing Ciliated Cells | SYAP1 | Early Response FOXJ1 high | GLO1 | HOPX high Squamous Cells | RNF170 |
| | | Ciliated Cells | | | |
| Developing Ciliated Cells | UBL5 | Early Response FOXJ1 high | EPPIN | HOPX high Squamous Cells | CALML3-AS1 |
| | | Ciliated Cells | | | |
| Developing Ciliated Cells | NAA20 | Early Response FOXJ1 high | NONO | HOPX high Squamous Cells | RNF149 |
| | | Ciliated Cells | | | |
| Developing Ciliated Cells | HMGN2 | Early Response FOXJ1 high | MYH14 | HOPX high Squamous Cells | GLTP |
| | | Ciliated Cells | | | |
| Developing Ciliated Cells | ACTB | Early Response FOXJ1 high | SERTAD2 | HOPX high Squamous Cells | TMEM40 |
| | | Ciliated Cells | | | |
| Developing Ciliated Cells | NAA38 | Early Response FOXJ1 high | BNIP3 | HOPX high Squamous Cells | MCL1 |
| | | Ciliated Cells | | | |
| Developing Ciliated Cells | TOB1 | Early Response FOXJ1 high | MXD4 | HOPX high Squamous Cells | CDC34 |
| | | Ciliated Cells | | | |
| Developing Ciliated Cells | PPP4R3B | Early Response FOXJ1 high | BRK1 | HOPX high Squamous Cells | LYPD3 |
| | | Ciliated Cells | | | |
| Developing Ciliated Cells | NDUFAB1 | Early Response FOXJ1 high | CFAP44 | HOPX high Squamous Cells | PIM1 |
| | | Ciliated Cells | | | |
| Developing Ciliated Cells | NARS | Early Response FOXJ1 high | SMIM22 | HOPX high Squamous Cells | MIEN1 |
| | | Ciliated Cells | | | |
| Developing Ciliated Cells | S100A11 | Early Response FOXJ1 high | IQCB1 | HOPX high Squamous Cells | NRBP1 |
| | | Ciliated Cells | | | |
| Developing Ciliated Cells | MDH1 | Early Response FOXJ1 high | C18orf25 | HOPX high Squamous Cells | RHOV |
| | | Ciliated Cells | | | |
| Developing Ciliated Cells | IDS | Early Response FOXJ1 high | MGMT | HOPX high Squamous Cells | CFL1 |
| | | Ciliated Cells | | | |
| Developing Ciliated Cells | C11orf58 | Early Response FOXJ1 high | AGL | HOPX high Squamous Cells | SDCBP |
| | | Ciliated Cells | | | |
| Developing Ciliated Cells | NDUFV2 | Early Response FOXJ1 high | SRSF3 | HOPX high Squamous Cells | GPR160 |
| | | Ciliated Cells | | | |
| Developing Ciliated Cells | UBB | Early Response FOXJ1 high | PSMD10 | HOPX high Squamous Cells | RDH13 |
| | | Ciliated Cells | | | |
| Developing Ciliated Cells | MYL12B | Early Response FOXJ1 high | ARRDC3 | HOPX high Squamous Cells | MARVELD3 |
| | | Ciliated Cells | | | |
| Developing Ciliated Cells | MOB1A | Early Response FOXJ1 high | RAB11FIP1 | HOPX high Squamous Cells | TMEM80 |
| | | Ciliated Cells | | | |
| Developing Ciliated Cells | ANXA7 | Early Response FOXJ1 high | LCOR | HOPX high Squamous Cells | RAB5A |
| | | Ciliated Cells | | | |
| Developing Ciliated Cells | PTMA | Early Response FOXJ1 high | RNF11 | HOPX high Squamous Cells | VSIG10L |
| | | Ciliated Cells | | | |
| Developing Ciliated Cells | C19orf70 | Early Response FOXJ1 high | RBL2 | HOPX high Squamous Cells | RRAGC |
| | | Ciliated Cells | | | |
| Developing Ciliated Cells | SELENOF | Early Response FOXJ1 high | FBXO31 | HOPX high Squamous Cells | ARHGEF10L |
| | | Ciliated Cells | | | |
| Developing Ciliated Cells | NAP1L1 | Early Response FOXJ1 high | TXLNA | HOPX high Squamous Cells | CAPZB |
| | | Ciliated Cells | | | |
| Developing Ciliated Cells | TMCO1 | Early Response FOXJ1 high | LRPAP1 | HOPX high Squamous Cells | AOC1 |
| | | Ciliated Cells | | | |
| Developing Ciliated Cells | PFDN5 | Early Response FOXJ1 high | CCDC181 | HOPX high Squamous Cells | MYZAP |
| | | Ciliated Cells | | | |
| Developing Ciliated Cells | C6orf62 | Early Response FOXJ1 high | ZCRB1 | HOPX high Squamous Cells | TICAM1 |
| | | Ciliated Cells | | | |
| Developing Ciliated Cells | SELENOH | Early Response FOXJ1 high | GSDMD | HOPX high Squamous Cells | DBNL |
| | | Ciliated Cells | | | |
| Developing Ciliated Cells | TPM3 | Early Response FOXJ1 high | SUMF2 | HOPX high Squamous Cells | FNDC3B |
| | | Ciliated Cells | | | |
| Developing Ciliated Cells | UQCRB | Early Response FOXJ1 high | ARL6 | HOPX high Squamous Cells | LRMP |
| | | Ciliated Cells | | | |
| Developing Ciliated Cells | ATP5F1C | Early Response FOXJ1 high | NLRP1 | HOPX high Squamous Cells | MERTK |
| | | Ciliated Cells | | | |
| Developing Ciliated Cells | BUD31 | Early Response FOXJ1 high | PACSIN2 | HOPX high Squamous Cells | MAP7D1 |
| | | Ciliated Cells | | | |
| Developing Ciliated Cells | BUD23 | Early Response FOXJ1 high | PMPCB | HOPX high Squamous Cells | SUPT4H1 |
| | | Ciliated Cells | | | |
| Developing Ciliated Cells | CANX | Early Response FOXJ1 high | SNX29 | HOPX high Squamous Cells | HIST1H2BF |
| | | Ciliated Cells | | | |
| Developing Ciliated Cells | SPCS1 | Early Response FOXJ1 high | NEDD8 | HOPX high Squamous Cells | NAA50 |
| | | Ciliated Cells | | | |
| Developing Ciliated Cells | COX6B1 | Early Response FOXJ1 high | SNX14 | HOPX high Squamous Cells | SECTM1 |
| | | Ciliated Cells | | | |
| Developing Ciliated Cells | SMDT1 | Early Response FOXJ1 high | CUEDC1 | HOPX high Squamous Cells | RIN3 |
| | | Ciliated Cells | | | |
| Developing Ciliated Cells | HIPK3 | Early Response FOXJ1 high | FHAD1 | HOPX high Squamous Cells | CAPN5 |
| | | Ciliated Cells | | | |
| Developing Ciliated Cells | TMEM59 | Early Response FOXJ1 high | FAM172A | HOPX high Squamous Cells | ELOVL6 |
| | | Ciliated Cells | | | |
| Developing Ciliated Cells | HMGN1 | Early Response FOXJ1 high | ARF4 | HOPX high Squamous Cells | HIST1H2BC |
| | | Ciliated Cells | | | |
| Developing Ciliated Cells | RBM3 | Early Response FOXJ1 high | IER2 | HOPX high Squamous Cells | CTDSP1 |
| | | Ciliated Cells | | | |
| Developing Ciliated Cells | TMEM14B | Early Response FOXJ1 high | SNCAIP | HOPX high Squamous Cells | MXI1 |
| | | Ciliated Cells | | | |
| Developing Ciliated Cells | RPS13 | Early Response FOXJ1 high | PEX2 | HOPX high Squamous Cells | EIF6 |
| | | Ciliated Cells | | | |
| Developing Ciliated Cells | FAM96B | Early Response FOXJ1 high | PJA2 | HOPX high Squamous Cells | CHMP1B |
| | | Ciliated Cells | | | |
| Developing Ciliated Cells | SET | Early Response FOXJ1 high | STX16 | HOPX high Squamous Cells | PTPRU |
| | | Ciliated Cells | | | |
| Developing Ciliated Cells | PSMA7 | Early Response FOXJ1 high | TLCD2 | HOPX high Squamous Cells | RPS27L |
| | | Ciliated Cells | | | |
| Developing Ciliated Cells | VPS35 | Early Response FOXJ1 high | PFDN6 | HOPX high Squamous Cells | MPZL3 |
| | | Ciliated Cells | | | |
| Developing Ciliated Cells | MPC2 | Early Response FOXJ1 high | LAMTOR4 | HOPX high Squamous Cells | OSTF1 |
| | | Ciliated Cells | | | |
| Developing Ciliated Cells | HNRNPC | Early Response FOXJ1 high | SPPL2A | HOPX high Squamous Cells | TAOK3 |
| | | Ciliated Cells | | | |
| Developing Ciliated Cells | RPL36AL | Early Response FOXJ1 high | PIK3R3 | HOPX high Squamous Cells | LAMP2 |
| | | Ciliated Cells | | | |
| Developing Ciliated Cells | C4orf3 | Early Response FOXJ1 high | USP22 | HOPX high Squamous Cells | HMOX1 |
| | | Ciliated Cells | | | |
| Developing Ciliated Cells | ZCRB1 | Early Response FOXJ1 high | WDFY1 | HOPX high Squamous Cells | ARHGDIA |
| | | Ciliated Cells | | | |
| Developing Ciliated Cells | RPL38 | Early Response FOXJ1 high | COPG1 | HOPX high Squamous Cells | SCAMP2 |
| | | Ciliated Cells | | | |
| Developing Ciliated Cells | ELOB | Early Response FOXJ1 high | OCIAD1 | HOPX high Squamous Cells | FBXW5 |
| | | Ciliated Cells | | | |
| Developing Ciliated Cells | SRSF9 | Early Response FOXJ1 high | RAB10 | HOPX high Squamous Cells | YIPF3 |
| | | Ciliated Cells | | | |
| Developing Ciliated Cells | NAP1L4 | Early Response FOXJ1 high | TNFAIP3 | HOPX high Squamous Cells | ITCH |
| | | Ciliated Cells | | | |
| Developing Ciliated Cells | ITM2B | Early Response FOXJ1 high | SMARCA2 | HOPX high Squamous Cells | RIT1 |
| | | Ciliated Cells | | | |
| Developing Ciliated Cells | 6-Mar | Early Response FOXJ1 high | PAPOLA | HOPX high Squamous Cells | RAB18 |
| | | Ciliated Cells | | | |
| Developing Ciliated Cells | NDUFB7 | Early Response FOXJ1 high | YWHAE | HOPX high Squamous Cells | IFNGR2 |
| | | Ciliated Cells | | | |
| Developing Ciliated Cells | SLAIN2 | Early Response FOXJ1 high | HYDIN | HOPX high Squamous Cells | CTDSP2 |
| | | Ciliated Cells | | | |
| Developing Ciliated Cells | GLO1 | Early Response FOXJ1 high | HADHB | HOPX high Squamous Cells | TSC22D4 |
| | | Ciliated Cells | | | |
| Developing Ciliated Cells | ANAPC16 | Early Response FOXJ1 high | DYNLL2 | HOPX high Squamous Cells | RASSF5 |
| | | Ciliated Cells | | | |
| Developing Ciliated Cells | LAPTM4A | Early Response FOXJ1 high | UBAP2L | HOPX high Squamous Cells | TINCR |
| | | Ciliated Cells | | | |
| Developing Ciliated Cells | MSI2 | Early Response FOXJ1 high | CCDC32 | HOPX high Squamous Cells | GOLGA7 |
| | | Ciliated Cells | | | |
| Developing Ciliated Cells | G3BP2 | Early Response FOXJ1 high | SAT2 | HOPX high Squamous Cells | FXYD5 |
| | | Ciliated Cells | | | |
| Developing Ciliated Cells | CDH1 | Early Response FOXJ1 high | C4orf47 | HOPX high Squamous Cells | AC004130.1 |
| | | Ciliated Cells | | | |
| Developing Ciliated Cells | PYURF | Early Response FOXJ1 high | BCAP31 | HOPX high Squamous Cells | NECTIN2 |
| | | Ciliated Cells | | | |
| Developing Ciliated Cells | PARK7 | Early Response FOXJ1 high | KRCC1 | HOPX high Squamous Cells | 8-Sep |
| | | Ciliated Cells | | | |
| Developing Ciliated Cells | FGD5-AS1 | Early Response FOXJ1 high | PCNX4 | HOPX high Squamous Cells | PLEKHJ1 |
| | | Ciliated Cells | | | |
| Developing Ciliated Cells | SRSF3 | Early Response FOXJ1 high | SLC23A2 | HOPX high Squamous Cells | PPCDC |
| | | Ciliated Cells | | | |
| Developing Ciliated Cells | CD46 | Early Response FOXJ1 high | LIMK2 | HOPX high Squamous Cells | CLDN23 |
| | | Ciliated Cells | | | |
| Developing Ciliated Cells | TM9SF2 | Early Response FOXJ1 high | ZFP36L2 | HOPX high Squamous Cells | EPHX3 |
| | | Ciliated Cells | | | |
| Developing Ciliated Cells | TCP1 | Early Response FOXJ1 high | GGA2 | HOPX high Squamous Cells | ELF3-AS1 |
| | | Ciliated Cells | | | |
| Developing Ciliated Cells | SLC25A5 | Early Response FOXJ1 high | TCTEX1D2 | HOPX high Squamous Cells | SUN2 |
| | | Ciliated Cells | | | |
| Developing Ciliated Cells | CD63 | Early Response FOXJ1 high | NDUFB5 | HOPX high Squamous Cells | ARL4D |
| | | Ciliated Cells | | | |
| Developing Ciliated Cells | NPTN | Early Response FOXJ1 high | HNRNPM | HOPX high Squamous Cells | CDK16 |
| | | Ciliated Cells | | | |
| Developing Ciliated Cells | DAZAP2 | Early Response FOXJ1 high | NAP1L4 | HOPX high Squamous Cells | FNBP1L |
| | | Ciliated Cells | | | |
| Developing Ciliated Cells | PSMA4 | Early Response FOXJ1 high | FAM81A | HOPX high Squamous Cells | ZNF706 |
| | | Ciliated Cells | | | |
| Developing Ciliated Cells | TOMM7 | Early Response FOXJ1 high | AKAP11 | HOPX high Squamous Cells | IST1 |
| | | Ciliated Cells | | | |
| Developing Ciliated Cells | GHITM | Early Response FOXJ1 high | GALNS | HOPX high Squamous Cells | REXO2 |
| | | Ciliated Cells | | | |
| Developing Ciliated Cells | SPATS2L | Early Response FOXJ1 high | ITGA3 | HOPX high Squamous Cells | RCC1 |
| | | Ciliated Cells | | | |
| Developing Ciliated Cells | GDI2 | Early Response FOXJ1 high | GLCCI1 | HOPX high Squamous Cells | ABI1 |
| | | Ciliated Cells | | | |
| Developing Ciliated Cells | UBL3 | Early Response FOXJ1 high | HNRNPU | HOPX high Squamous Cells | GTPBP2 |
| | | Ciliated Cells | | | |
| Developing Ciliated Cells | CAST | Early Response FOXJ1 high | CAPN2 | HOPX high Squamous Cells | LINC01559 |
| | | Ciliated Cells | | | |
| Developing Ciliated Cells | PTBP3 | Early Response FOXJ1 high | FXYD3 | HOPX high Squamous Cells | TRIM7 |
| | | Ciliated Cells | | | |
| Developing Ciliated Cells | RABGAP1L | Early Response FOXJ1 high | ZFP36 | HOPX high Squamous Cells | FAM89B |
| | | Ciliated Cells | | | |
| Developing Ciliated Cells | CSTB | Early Response FOXJ1 high | FAM120B | HOPX high Squamous Cells | UBE2J1 |
| | | Ciliated Cells | | | |
| Developing Ciliated Cells | CLCN3 | Early Response FOXJ1 high | SMARCA5 | HOPX high Squamous Cells | CBX4 |
| | | Ciliated Cells | | | |
| Developing Ciliated Cells | HSP90AA1 | Early Response FOXJ1 high | CYBA | HOPX high Squamous Cells | CCNYL1 |
| | | Ciliated Cells | | | |
| Developing Ciliated Cells | TMF1 | Early Response FOXJ1 high | POLR2H | HOPX high Squamous Cells | INPP4B |
| | | Ciliated Cells | | | |
| Developing Ciliated Cells | TAX1BP1 | Early Response FOXJ1 high | ZBBX | HOPX high Squamous Cells | MPP5 |
| | | Ciliated Cells | | | |
| Developing Ciliated Cells | RTN3 | Early Response FOXJ1 high | AGO1 | HOPX high Squamous Cells | STX10 |
| | | Ciliated Cells | | | |
| Developing Ciliated Cells | CHCHD2 | Early Response FOXJ1 high | UBXN1 | HOPX high Squamous Cells | VLDLR |
| | | Ciliated Cells | | | |
| Developing Ciliated Cells | YWHAZ | Early Response FOXJ1 high | EVI5 | HOPX high Squamous Cells | RAP2B |
| | | Ciliated Cells | | | |
| Developing Ciliated Cells | EID1 | Early Response FOXJ1 high | SUN1 | HOPX high Squamous Cells | ZNF554 |
| | | Ciliated Cells | | | |
| Developing Ciliated Cells | COX5A | Early Response FOXJ1 high | TTLL1 | HOPX high Squamous Cells | TMCC3 |
| | | Ciliated Cells | | | |
| Developing Ciliated Cells | NDUFS5 | Early Response FOXJ1 high | ZNF264 | HOPX high Squamous Cells | AGFG2 |
| | | Ciliated Cells | | | |
| Developing Ciliated Cells | VDAC3 | Early Response FOXJ1 high | PLPP2 | HOPX high Squamous Cells | HEBP1 |
| | | Ciliated Cells | | | |
| Developing Ciliated Cells | 7-Sep | Early Response FOXJ1 high | GSTA3 | HOPX high Squamous Cells | NATD1 |
| | | Ciliated Cells | | | |
| Developing Ciliated Cells | ACTR2 | Early Response FOXJ1 high | SRP68 | HOPX high Squamous Cells | FAM120AOS |
| | | Ciliated Cells | | | |
| Developing Ciliated Cells | PDCD6IP | Early Response FOXJ1 high | MECOM | HOPX high Squamous Cells | RANBP9 |
| | | Ciliated Cells | | | |
| Developing Ciliated Cells | PAPOLA | Early Response FOXJ1 high | TIPARP | HOPX high Squamous Cells | SQOR |
| | | Ciliated Cells | | | |
| Developing Ciliated Cells | CLDN7 | Early Response FOXJ1 high | ANKFN1 | HOPX high Squamous Cells | GATA3 |
| | | Ciliated Cells | | | |
| Developing Ciliated Cells | HNRNPF | Early Response FOXJ1 high | C19orf70 | HOPX high Squamous Cells | NXN |
| | | Ciliated Cells | | | |
| Developing Ciliated Cells | RPL15 | Early Response FOXJ1 high | C2CD3 | HOPX high Squamous Cells | USF2 |
| | | Ciliated Cells | | | |
| Developing Ciliated Cells | ATP5F1A | Early Response FOXJ1 high | MPV17L | HOPX high Squamous Cells | FKBP4 |
| | | Ciliated Cells | | | |
| Developing Ciliated Cells | CASC4 | Early Response FOXJ1 high | YME1L1 | HOPX high Squamous Cells | LINC01705 |
| | | Ciliated Cells | | | |
| Developing Ciliated Cells | PPP2CB | Early Response FOXJ1 high | ICK | HOPX high Squamous Cells | ATP6V1F |
| | | Ciliated Cells | | | |
| Developing Ciliated Cells | COX411 | Early Response FOXJ1 high | H2AFJ | HOPX high Squamous Cells | UBE2D1 |
| | | Ciliated Cells | | | |
| Developing Ciliated Cells | SIVA1 | Early Response FOXJ1 high | CAND1 | HOPX high Squamous Cells | UBE2D2 |
| | | Ciliated Cells | | | |
| Developing Ciliated Cells | UGP2 | Early Response FOXJ1 high | STARD7 | HOPX high Squamous Cells | MMADHC |
| | | Ciliated Cells | | | |
| Developing Ciliated Cells | EZR | Early Response FOXJ1 high | SLC25A3 | HOPX high Squamous Cells | WSB2 |
| | | Ciliated Cells | | | |
| Developing Ciliated Cells | ZNHIT1 | Early Response FOXJ1 high | RAB7A | HOPX high Squamous Cells | PPM1A |
| | | Ciliated Cells | | | |
| Developing Ciliated Cells | TSC22D1 | Early Response FOXJ1 high | MED25 | HOPX high Squamous Cells | VASN |
| | | Ciliated Cells | | | |
| Developing Ciliated Cells | CPNE3 | Early Response FOXJ1 high | RPS4X | HOPX high Squamous Cells | TGFA |
| | | Ciliated Cells | | | |
| Developing Ciliated Cells | TC2N | Early Response FOXJ1 high | DDX5 | HOPX high Squamous Cells | RECQL5 |
| | | Ciliated Cells | | | |
| Developing Ciliated Cells | SMARCA5 | Early Response FOXJ1 high | SLC20A1 | HOPX high Squamous Cells | STK38 |
| | | Ciliated Cells | | | |
| Developing Ciliated Cells | BSG | Early Response FOXJ1 high | USO1 | HOPX high Squamous Cells | LRRC8A |
| | | Ciliated Cells | | | |
| Developing Ciliated Cells | FTH1 | Early Response FOXJ1 high | CIAO1 | HOPX high Squamous Cells | PCMTD1 |
| | | Ciliated Cells | | | |
| Developing Ciliated Cells | MRPS21 | Early Response FOXJ1 high | NOLC1 | HOPX high Squamous Cells | MGAT4B |
| | | Ciliated Cells | | | |
| Developing Ciliated Cells | MRPL41 | Early Response FOXJ1 high | CLN5 | HOPX high Squamous Cells | TOM1L2 |
| | | Ciliated Cells | | | |
| Developing Ciliated Cells | EIF1 | Early Response FOXJ1 high | CDKN1A | HOPX high Squamous Cells | SIRT7 |
| | | Ciliated Cells | | | |
| Developing Ciliated Cells | HSPA9 | Early Response FOXJ1 high | NFIA | HOPX high Squamous Cells | HIGD1A |
| | | Ciliated Cells | | | |
| Developing Ciliated Cells | SDC4 | Early Response FOXJ1 high | EFCAB14 | HOPX high Squamous Cells | CAPN14 |
| | | Ciliated Cells | | | |
| Developing Ciliated Cells | SCP2 | Early Response FOXJ1 high | NDUFA2 | HOPX high Squamous Cells | PTP4A1 |
| | | Ciliated Cells | | | |
| Developing Ciliated Cells | ATXN7L3B | Early Response FOXJ1 high | TAF7 | HOPX high Squamous Cells | FAM49B |
| | | Ciliated Cells | | | |
| Developing Ciliated Cells | BCLAF1 | Early Response FOXJ1 high | NR2F6 | HOPX high Squamous Cells | NBDY |
| | | Ciliated Cells | | | |
| Developing Ciliated Cells | C1orf43 | Early Response FOXJ1 high | POLR2F | HOPX high Squamous Cells | SPINT1-AS1 |
| | | Ciliated Cells | | | |
| Developing Ciliated Cells | MATR3 | Early Response FOXJ1 high | ATP5PB | HOPX high Squamous Cells | ARPC4 |
| | | Ciliated Cells | | | |
| Developing Ciliated Cells | CLTA | Early Response FOXJ1 high | DCAF7 | HOPX high Squamous Cells | SH2D1B |
| | | Ciliated Cells | | | |
| Developing Ciliated Cells | EIF4A2 | Early Response FOXJ1 high | ABRAXAS1 | HOPX high Squamous Cells | GBP2 |
| | | Ciliated Cells | | | |
| Developing Ciliated Cells | 2-Sep | Early Response FOXJ1 high | SPART | HOPX high Squamous Cells | RHOD |
| | | Ciliated Cells | | | |
| Developing Ciliated Cells | RAB14 | Early Response FOXJ1 high | PPP1R15B | HOPX high Squamous Cells | SH3PXD2A- |
| | | Ciliated Cells | | | AS1 |
| Developing Ciliated Cells | NFE2L2 | Early Response FOXJ1 high | TFF3 | HOPX high Squamous Cells | TNFRSF1A |
| | | Ciliated Cells | | | |
| Developing Ciliated Cells | DDX3X | Early Response FOXJ1 high | RASEF | HOPX high Squamous Cells | RASSF7 |
| | | Ciliated Cells | | | |
| Developing Ciliated Cells | ZNF24 | Early Response FOXJ1 high | MAPK10 | HOPX high Squamous Cells | TTLL12 |
| | | Ciliated Cells | | | |
| Developing Ciliated Cells | COX8A | Early Response FOXJ1 high | PBRM1 | HOPX high Squamous Cells | CTNNBIP1 |
| | | Ciliated Cells | | | |
| Developing Ciliated Cells | RPN1 | Early Response FOXJ1 high | ADGRE5 | HOPX high Squamous Cells | CAPZA1 |
| | | Ciliated Cells | | | |
| Developing Ciliated Cells | RTN4 | Early Response FOXJ1 high | ALS2CR12 | HOPX high Squamous Cells | RBMS2 |
| | | Ciliated Cells | | | |
| Developing Ciliated Cells | DNAJA2 | Early Response FOXJ1 high | AARS | HOPX high Squamous Cells | KCTD5 |
| | | Ciliated Cells | | | |
| Developing Ciliated Cells | GPBP1 | Early Response FOXJ1 high | KPNA6 | HOPX high Squamous Cells | NRAS |
| | | Ciliated Cells | | | |
| Developing Ciliated Cells | PSMB1 | Early Response FOXJ1 high | TKT | HOPX high Squamous Cells | RNF7 |
| | | Ciliated Cells | | | |
| Developing Ciliated Cells | PDCD4 | Early Response FOXJ1 high | TNPO2 | HOPX high Squamous Cells | UBE2I |
| | | Ciliated Cells | | | |
| Developing Ciliated Cells | ATP5PB | Early Response FOXJ1 high | PRMT2 | HOPX high Squamous Cells | BOK |
| | | Ciliated Cells | | | |
| Developing Ciliated Cells | KTN1 | Early Response FOXJ1 high | CEP120 | HOPX high Squamous Cells | CD151 |
| | | Ciliated Cells | | | |
| Developing Ciliated Cells | PSME1 | Early Response FOXJ1 high | MAT2B | HOPX high Squamous Cells | POLR2K |
| | | Ciliated Cells | | | |
| Developing Ciliated Cells | CCT3 | Early Response FOXJ1 high | DIDO1 | HOPX high Squamous Cells | PDZK1IP1 |
| | | Ciliated Cells | | | |
| Developing Ciliated Cells | SYPL1 | Early Response FOXJ1 high | SLC30A5 | HOPX high Squamous Cells | MAPK13 |
| | | Ciliated Cells | | | |
| Developing Ciliated Cells | ISCU | Early Response FOXJ1 high | RPS6KA5 | HOPX high Squamous Cells | NOP10 |
| | | Ciliated Cells | | | |
| Developing Ciliated Cells | BCAP31 | Early Response FOXJ1 high | PTBP1 | HOPX high Squamous Cells | DCTN5 |
| | | Ciliated Cells | | | |
| Developing Ciliated Cells | CCPG1 | Early Response FOXJ1 high | SELENOF | HOPX high Squamous Cells | ABHD17C |
| | | Ciliated Cells | | | |
| Developing Ciliated Cells | IRF2BP2 | Early Response FOXJ1 high | IPO11 | HOPX high Squamous Cells | INPP5K |
| | | Ciliated Cells | | | |
| Developing Ciliated Cells | EMC4 | Early Response FOXJ1 high | PFDN5 | HOPX high Squamous Cells | OXSR1 |
| | | Ciliated Cells | | | |
| Developing Ciliated Cells | SSBP1 | Early Response FOXJ1 high | DNAH6 | HOPX high Squamous Cells | VDAC2 |
| | | Ciliated Cells | | | |
| Developing Ciliated Cells | NDUFA2 | Early Response FOXJ1 high | MECP2 | HOPX high Squamous Cells | RRAS |
| | | Ciliated Cells | | | |
| Developing Ciliated Cells | CNBP | Early Response FOXJ1 high | MFSD14B | HOPX high Squamous Cells | TRNAU1AP |
| | | Ciliated Cells | | | |
| Developing Ciliated Cells | TMEM30A | Early Response FOXJ1 high | DUSP4 | HOPX high Squamous Cells | NEU1 |
| | | Ciliated Cells | | | |
| Developing Ciliated Cells | WDR45B | Early Response FOXJ1 high | KIAA1257 | HOPX high Squamous Cells | SH2D3A |
| | | Ciliated Cells | | | |
| Developing Ciliated Cells | OCIAD1 | Early Response FOXJ1 high | PAFAH1B1 | HOPX high Squamous Cells | ATP6V1H |
| | | Ciliated Cells | | | |
| Developing Ciliated Cells | DHX30 | Early Response FOXJ1 high | POLD3 | HOPX high Squamous Cells | EREG |
| | | Ciliated Cells | | | |
| Developing Ciliated Cells | YBX3 | Early Response FOXJ1 high | MAN1A2 | HOPX high Squamous Cells | JOSD1 |
| | | Ciliated Cells | | | |
| Developing Secretory and | ADAM28 | Early Response FOXJ1 high | STIP1 | HOPX high Squamous Cells | SIPA1L2 |
| Goblet Cells | | Ciliated Cells | | | |
| Developing Secretory and | FAT2 | Early Response FOXJ1 high | G3BP1 | HOPX high Squamous Cells | CANT1 |
| Goblet Cells | | Ciliated Cells | | | |
| Developing Secretory and | SRRM2 | Early Response FOXJ1 high | OS9 | HOPX high Squamous Cells | MAFG |
| Goblet Cells | | Ciliated Cells | | | |
| Developing Secretory and | MALAT1 | Early Response FOXJ1 high | MRPL42 | HOPX high Squamous Cells | MAPKAPK2 |
| Goblet Cells | | Ciliated Cells | | | |
| Early Response FOXJ1 high | CAPS | Early Response FOXJ1 high | DAPP1 | HOPX high Squamous Cells | ARHGAP10 |
| Ciliated Cells | | Ciliated Cells | | | |
| Early Response FOXJ1 high | RRAD | Early Response FOXJ1 high | ANAPC16 | HOPX high Squamous Cells | 5-Mar |
| Ciliated Cells | | Ciliated Cells | | | |
| Early Response FOXJ1 high | CTGF | Early Response FOXJ1 high | TMEM30A | HOPX high Squamous Cells | MRPS18A |
| Ciliated Cells | | Ciliated Cells | | | |
| Early Response FOXJ1 high | ALDH3B1 | Early Response FOXJ1 high | IARS | HOPX high Squamous Cells | RNF223 |
| Ciliated Cells | | Ciliated Cells | | | |
| Early Response FOXJ1 high | TPPP3 | Early Response FOXJ1 high | PSMD8 | HOPX high Squamous Cells | GBA |
| Ciliated Cells | | Ciliated Cells | | | |
| Early Response FOXJ1 high | FAM216B | Early Response FOXJ1 high | KIAA1841 | HOPX high Squamous Cells | C15orf39 |
| Ciliated Cells | | Ciliated Cells | | | |
| Early Response FOXJ1 high | TUBB4B | Early Response FOXJ1 high | DUOXA1 | HOPX high Squamous Cells | CDKN2A |
| Ciliated Cells | | Ciliated Cells | | | |
| Early Response FOXJ1 high | C20orf85 | Early Response FOXJ1 high | LMAN1 | HOPX high Squamous Cells | DYNLT3 |
| Ciliated Cells | | Ciliated Cells | | | |
| Early Response FOXJ1 high | DUSP1 | Early Response FOXJ1 high | EMP2 | HOPX high Squamous Cells | BLNK |
| Ciliated Cells | | Ciliated Cells | | | |
| Early Response FOXJ1 high | ZMYND10 | Early Response FOXJ1 high | PDE4DIP | HOPX high Squamous Cells | MAP3K9 |
| Ciliated Cells | | Ciliated Cells | | | |
| Early Response FOXJ1 high | TUBA1A | Early Response FOXJ1 high | PSMB7 | HOPX high Squamous Cells | LTBR |
| Ciliated Cells | | Ciliated Cells | | | |
| Early Response FOXJ1 high | IGFBP7 | Early Response FOXJ1 high | LARP4B | HOPX high Squamous Cells | LMBRD1 |
| Ciliated Cells | | Ciliated Cells | | | |
| Early Response FOXJ1 high | PRDX5 | Early Response FOXJ1 high | UBE2A | HOPX high Squamous Cells | SCRIB |
| Ciliated Cells | | Ciliated Cells | | | |
| Early Response FOXJ1 high | AC007906.2 | Early Response FOXJ1 high | TTC39C | HOPX high Squamous Cells | TFG |
| Ciliated Cells | | Ciliated Cells | | | |
| Early Response FOXJ1 high | MAP6 | Early Response FOXJ1 high | POLR2B | HOPX high Squamous Cells | RELA |
| Ciliated Cells | | Ciliated Cells | | | |
| Early Response FOXJ1 high | CCDC170 | Early Response FOXJ1 high | NCALD | HOPX high Squamous Cells | FMR1 |
| Ciliated Cells | | Ciliated Cells | | | |
| Early Response FOXJ1 high | SPAG6 | Early Response FOXJ1 high | HNRNPH3 | HOPX high Squamous Cells | OSER1 |
| Ciliated Cells | | Ciliated Cells | | | |
| Early Response FOXJ1 high | CD59 | Early Response FOXJ1 high | NFIC | HOPX high Squamous Cells | C1GALT1C1 |
| Ciliated Cells | | Ciliated Cells | | | |
| Early Response FOXJ1 high | SPATA18 | Early Response FOXJ1 high | ATXN10 | HOPX high Squamous Cells | PSEN1 |
| Ciliated Cells | | Ciliated Cells | | | |
| Early Response FOXJ1 high | C5orf49 | Early Response FOXJ1 high | STK11IP | HOPX high Squamous Cells | PHF23 |
| Ciliated Cells | | Ciliated Cells | | | |
| Early Response FOXJ1 high | C9orf24 | Early Response FOXJ1 high | GLOD4 | HOPX high Squamous Cells | VPS26A |
| Ciliated Cells | | Ciliated Cells | | | |
| Early Response FOXJ1 high | CES1 | Early Response FOXJ1 high | PHB | HOPX high Squamous Cells | LAD1 |
| Ciliated Cells | | Ciliated Cells | | | |
| Early Response FOXJ1 high | FOXJ1 | Early Response FOXJ1 high | FAM213A | HOPX high Squamous Cells | CCDC9B |
| Ciliated Cells | | Ciliated Cells | | | |
| Early Response FOXJ1 high | RSPH1 | Early Response FOXJ1 high | PUM1 | HOPX high Squamous Cells | PLCD3 |
| Ciliated Cells | | Ciliated Cells | | | |
| Early Response FOXJ1 high | JUN | Early Response FOXJ1 high | CCDC173 | HOPX high Squamous Cells | TSPAN14 |
| Ciliated Cells | | Ciliated Cells | | | |
| Early Response FOXJ1 high | CLDN3 | Early Response FOXJ1 high | ACLY | HOPX high Squamous Cells | NFKB2 |
| Ciliated Cells | | Ciliated Cells | | | |
| Early Response FOXJ1 high | ALOX15 | Early Response FOXJ1 high | ANXA11 | HOPX high Squamous Cells | SSH3 |
| Ciliated Cells | | Ciliated Cells | | | |
| Early Response FOXJ1 high | UBXN10 | Early Response FOXJ1 high | ZC3H14 | HOPX high Squamous Cells | USP53 |
| Ciliated Cells | | Ciliated Cells | | | |
| Early Response FOXJ1 high | NWD1 | Early Response FOXJ1 high | UQCRC2 | HOPX high Squamous Cells | JARID2 |
| Ciliated Cells | | Ciliated Cells | | | |
| Early Response FOXJ1 high | STOML3 | Early Response FOXJ1 high | PRRC2B | HOPX high Squamous Cells | MMP14 |
| Ciliated Cells | | Ciliated Cells | | | |
| Early Response FOXJ1 high | BASP1 | Early Response FOXJ1 high | CCDC78 | HOPX high Squamous Cells | ADAM10 |
| Ciliated Cells | | Ciliated Cells | | | |
| Early Response FOXJ1 high | DNAJA4 | Early Response FOXJ1 high | CCDC47 | HOPX high Squamous Cells | ABRACL |
| Ciliated Cells | | Ciliated Cells | | | |
| Early Response FOXJ1 high | CKB | Early Response FOXJ1 high | METTL16 | HOPX high Squamous Cells | CHMP1A |
| Ciliated Cells | | Ciliated Cells | | | |
| Early Response FOXJ1 high | TEKT1 | Early Response FOXJ1 high | HMGXB3 | HOPX high Squamous Cells | UBE2W |
| Ciliated Cells | | Ciliated Cells | | | |
| Early Response FOXJ1 high | ENPP5 | Early Response FOXJ1 high | GSS | HOPX high Squamous Cells | RUNX2 |
| Ciliated Cells | | Ciliated Cells | | | |
| Early Response FOXJ1 high | CETN2 | Early Response FOXJ1 high | DGKH | HOPX high Squamous Cells | PLEKHM2 |
| Ciliated Cells | | Ciliated Cells | | | |
| Early Response FOXJ1 high | ROPN1L | Early Response FOXJ1 high | ALDH3A2 | HOPX high Squamous Cells | FAM32A |
| Ciliated Cells | | Ciliated Cells | | | |
| Early Response FOXJ1 high | LDLRAD1 | Early Response FOXJ1 high | RYBP | HOPX high Squamous Cells | RHOT1 |
| Ciliated Cells | | Ciliated Cells | | | |
| Early Response FOXJ1 high | UCP2 | Early Response FOXJ1 high | OTUD7B | HOPX high Squamous Cells | PPP1CA |
| Ciliated Cells | | Ciliated Cells | | | |
| Early Response FOXJ1 high | SLC7A2 | Early Response FOXJ1 high | RB1CC1 | HOPX high Squamous Cells | LINC01269 |
| Ciliated Cells | | Ciliated Cells | | | |
| Early Response FOXJ1 high | PROM1 | Early Response FOXJ1 high | SYTL1 | HOPX high Squamous Cells | AP3S1 |
| Ciliated Cells | | Ciliated Cells | | | |
| Early Response FOXJ1 high | CCDC113 | Early Response FOXJ1 high | HSPA9 | HOPX high Squamous Cells | STX18 |
| Ciliated Cells | | Ciliated Cells | | | |
| Early Response FOXJ1 high | IGFBP2 | Early Response FOXJ1 high | DYNC2LI1 | HOPX high Squamous Cells | KRT6A |
| Ciliated Cells | | Ciliated Cells | | | |
| Early Response FOXJ1 high | CCDC80 | Early Response FOXJ1 high | CALM3 | HOPX high Squamous Cells | ACAA1 |
| Ciliated Cells | | Ciliated Cells | | | |
| Early Response FOXJ1 high | RSPH4A | Early Response FOXJ1 high | KPNB1 | HOPX high Squamous Cells | ATXN7L3 |
| Ciliated Cells | | Ciliated Cells | | | |
| Early Response FOXJ1 high | NQ01 | Early Response FOXJ1 high | TMEM230 | HOPX high Squamous Cells | QKI |
| Ciliated Cells | | Ciliated Cells | | | |
| Early Response FOXJ1 high | GADD45B | Early Response FOXJ1 high | TEX26 | HOPX high Squamous Cells | SOWAHB |
| Ciliated Cells | | Ciliated Cells | | | |
| Early Response FOXJ1 high | CIB1 | Early Response FOXJ1 high | LRBA | HOPX high Squamous Cells | MOSPD1 |
| Ciliated Cells | | Ciliated Cells | | | |
| Early Response FOXJ1 high | LRRC10B | Early Response FOXJ1 high | ZMAT2 | HOPX high Squamous Cells | MAF |
| Ciliated Cells | | Ciliated Cells | | | |
| Early Response FOXJ1 high | CYR61 | Early Response FOXJ1 high | WDR90 | HOPX high Squamous Cells | C12orf29 |
| Ciliated Cells | | Ciliated Cells | | | |
| Early Response FOXJ1 high | TMEM190 | Early Response FOXJ1 high | DNAJC5 | HOPX high Squamous Cells | RNF181 |
| Ciliated Cells | | Ciliated Cells | | | |
| Early Response FOXJ1 high | BTG2 | Early Response FOXJ1 high | CSNK1G2 | HOPX high Squamous Cells | TMUB2 |
| Ciliated Cells | | Ciliated Cells | | | |
| Early Response FOXJ1 high | HSPA1B | Early Response FOXJ1 high | HAT1 | HOPX high Squamous Cells | RNF139 |
| Ciliated Cells | | Ciliated Cells | | | |
| Early Response FOXJ1 high | CYP4B1 | Early Response FOXJ1 high | AFTPH | HOPX high Squamous Cells | CPEB2 |
| Ciliated Cells | | Ciliated Cells | | | |
| Early Response FOXJ1 high | ERICH3 | Early Response FOXJ1 high | PLXNB1 | HOPX high Squamous Cells | HSPB1 |
| Ciliated Cells | | Ciliated Cells | | | |
| Early Response FOXJ1 high | NFE2L1 | Early Response FOXJ1 high | UBL5 | HOPX high Squamous Cells | CYTOR |
| Ciliated Cells | | Ciliated Cells | | | |
| Early Response FOXJ1 high | FOS | Early Response FOXJ1 high | RABL6 | HOPX high Squamous Cells | PPP2CA |
| Ciliated Cells | | Ciliated Cells | | | |
| Early Response FOXJ1 high | RHOB | Early Response FOXJ1 high | FAM104B | HOPX high Squamous Cells | LSR |
| Ciliated Cells | | Ciliated Cells | | | |
| Early Response FOXJ1 high | IGFBP5 | Early Response FOXJ1 high | KARS | HOPX high Squamous Cells | RGS10 |
| Ciliated Cells | | Ciliated Cells | | | |
| Early Response FOXJ1 high | KIF3B | Early Response FOXJ1 high | EDF1 | HOPX high Squamous Cells | LAMTOR1 |
| Ciliated Cells | | Ciliated Cells | | | |
| Early Response FOXJ1 high | LRRC23 | Early Response FOXJ1 high | ERBB2 | HOPX high Squamous Cells | PNPLA2 |
| Ciliated Cells | | Ciliated Cells | | | |
| Early Response FOXJ1 high | SNTN | Early Response FOXJ1 high | DAP3 | HOPX high Squamous Cells | VSIG10 |
| Ciliated Cells | | Ciliated Cells | | | |
| Early Response FOXJ1 high | UNC119B | Early Response FOXJ1 high | CSMD1 | HOPX high Squamous Cells | GPR153 |
| Ciliated Cells | | Ciliated Cells | | | |
| Early Response FOXJ1 high | WDR54 | Early Response FOXJ1 high | MTPN | HOPX high Squamous Cells | LINC02448 |
| Ciliated Cells | | Ciliated Cells | | | |
| Early Response FOXJ1 high | CDHR3 | Early Response FOXJ1 high | SPINT2 | HOPX high Squamous Cells | AMOTL2 |
| Ciliated Cells | | Ciliated Cells | | | |
| Early Response FOXJ1 high | WDR78 | Early Response FOXJ1 high | HNRNPA2B1 | HOPX high Squamous Cells | SP6 |
| Ciliated Cells | | Ciliated Cells | | | |
| Early Response FOXJ1 high | PKIG | Early Response FOXJ1 high | SNRNP200 | HOPX high Squamous Cells | ESAM |
| Ciliated Cells | | Ciliated Cells | | | |
| Early Response FOXJ1 high | LRP11 | Early Response FOXJ1 high | CTR9 | HOPX high Squamous Cells | GULP1 |
| Ciliated Cells | | Ciliated Cells | | | |
| Early Response FOXJ1 high | VWA3B | Early Response FOXJ1 high | SMAD2 | HOPX high Squamous Cells | RGS17 |
| Ciliated Cells | | Ciliated Cells | | | |
| Early Response FOXJ1 high | TNFAIP8L1 | Early Response FOXJ1 high | ECHS1 | HOPX high Squamous Cells | NTAN1 |
| Ciliated Cells | | Ciliated Cells | | | |
| Early Response FOXJ1 high | GDF15 | Early Response FOXJ1 high | BMI1 | HOPX high Squamous Cells | TNKS1BP1 |
| Ciliated Cells | | Ciliated Cells | | | |
| Early Response FOXJ1 high | HIPK1 | Early Response FOXJ1 high | FBXO7 | HOPX high Squamous Cells | RELB |
| Ciliated Cells | | Ciliated Cells | | | |
| Early Response FOXJ1 high | ZNF664 | Early Response FOXJ1 high | YAP1 | HOPX high Squamous Cells | SNX8 |
| Ciliated Cells | | Ciliated Cells | | | |
| Early Response FOXJ1 high | PIFO | Early Response FOXJ1 high | NDUFV1 | HOPX high Squamous Cells | PITPNC1 |
| Ciliated Cells | | Ciliated Cells | | | |
| Early Response FOXJ1 high | SAMHD1 | Early Response FOXJ1 high | AZI2 | HOPX high Squamous Cells | BCL9L |
| Ciliated Cells | | Ciliated Cells | | | |
| Early Response FOXJ1 high | ABHD2 | Early Response FOXJ1 high | NIN | HOPX high Squamous Cells | YTHDF3 |
| Ciliated Cells | | Ciliated Cells | | | |
| Early Response FOXJ1 high | CDS1 | Early Response FOXJ1 high | FAM219B | HOPX high Squamous Cells | VPS4A |
| Ciliated Cells | | Ciliated Cells | | | |
| Early Response FOXJ1 high | IFT57 | Early Response FOXJ1 high | CNTRL | HOPX high Squamous Cells | DCTN6 |
| Ciliated Cells | | Ciliated Cells | | | |
| Early Response FOXJ1 high | SLC44A4 | Early Response FOXJ1 high | FAM208A | HOPX high Squamous Cells | BCL10 |
| Ciliated Cells | | Ciliated Cells | | | |
| Early Response FOXJ1 high | AK7 | Early Response FOXJ1 high | CTSL | HOPX high Squamous Cells | GLMP |
| Ciliated Cells | | Ciliated Cells | | | |
| Early Response FOXJ1 high | EZR | Early Response FOXJ1 high | HSD17B4 | HOPX high Squamous Cells | ABHD12 |
| Ciliated Cells | | Ciliated Cells | | | |
| Early Response FOXJ1 high | KLHL6 | Early Response FOXJ1 high | YPEL5 | HOPX high Squamous Cells | TMEM79 |
| Ciliated Cells | | Ciliated Cells | | | |
| Early Response FOXJ1 high | SARAF | Early Response FOXJ1 high | RIMKLB | HOPX high Squamous Cells | CDC42EP1 |
| Ciliated Cells | | Ciliated Cells | | | |
| Early Response FOXJ1 high | DYNLL1 | Early Response FOXJ1 high | SLC35E1 | HOPX high Squamous Cells | RHOG |
| Ciliated Cells | | Ciliated Cells | | | |
| Early Response FOXJ1 high | SPEF1 | Early Response FOXJ1 high | MATR3 | HOPX high Squamous Cells | AMDHD2 |
| Ciliated Cells | | Ciliated Cells | | | |
| Early Response FOXJ1 high | CHST9 | Early Response FOXJ1 high | CUL1 | HOPX high Squamous Cells | STAP2 |
| Ciliated Cells | | Ciliated Cells | | | |
| Early Response FOXJ1 high | PACRG | Early Response FOXJ1 high | DNAJC3 | HOPX high Squamous Cells | DDA1 |
| Ciliated Cells | | Ciliated Cells | | | |
| Early Response FOXJ1 high | SAXO2 | Early Response FOXJ1 high | CAPN7 | HOPX high Squamous Cells | ELOC |
| Ciliated Cells | | Ciliated Cells | | | |
| Early Response FOXJ1 high | DNALI1 | Early Response FOXJ1 high | IQGAP1 | HOPX high Squamous Cells | AP2S1 |
| Ciliated Cells | | Ciliated Cells | | | |
| Early Response FOXJ1 high | HSPA1A | Early Response FOXJ1 high | COX8A | HOPX high Squamous Cells | DBP |
| Ciliated Cells | | Ciliated Cells | | | |
| Early Response FOXJ1 high | NUDC | Early Response FOXJ1 high | ARHGAP32 | HOPX high Squamous Cells | VMP1 |
| Ciliated Cells | | Ciliated Cells | | | |
| Early Response FOXJ1 high | FABP6 | Early Response FOXJ1 high | CYC1 | HOPX high Squamous Cells | RAPGEF3 |
| Ciliated Cells | | Ciliated Cells | | | |
| Early Response FOXJ1 high | TSPAN6 | Early Response FOXJ1 high | KANSL1L | HOPX high Squamous Cells | PPP4C |
| Ciliated Cells | | Ciliated Cells | | | |
| Early Response FOXJ1 high | TSPAN1 | Early Response FOXJ1 high | WDR1 | HOPX high Squamous Cells | PPP1R13L |
| Ciliated Cells | | Ciliated Cells | | | |
| Early Response FOXJ1 high | MAP1A | Early Response FOXJ1 high | ELF1 | HOPX high Squamous Cells | APH1A |
| Ciliated Cells | | Ciliated Cells | | | |
| Early Response FOXJ1 high | ANKRD37 | Early Response FOXJ1 high | XRCC6 | HOPX high Squamous Cells | SLC9A1 |
| Ciliated Cells | | Ciliated Cells | | | |
| Early Response FOXJ1 high | UFC1 | Early Response FOXJ1 high | CIR1 | HOPX high Squamous Cells | STX3 |
| Ciliated Cells | | Ciliated Cells | | | |
| Early Response FOXJ1 high | TMBIM6 | Early Response FOXJ1 high | FXR1 | HOPX high Squamous Cells | ALS2CL |
| Ciliated Cells | | Ciliated Cells | | | |
| Early Response FOXJ1 high | JPT2 | Early Response FOXJ1 high | CCDC34 | HOPX high Squamous Cells | MUL1 |
| Ciliated Cells | | Ciliated Cells | | | |
| Early Response FOXJ1 high | IFT22 | Early Response FOXJ1 high | LYN | HOPX high Squamous Cells | PLEKHF1 |
| Ciliated Cells | | Ciliated Cells | | | |
| Early Response FOXJ1 high | FAM92B | Early Response FOXJ1 high | ATP6V1D | HOPX high Squamous Cells | TAP2 |
| Ciliated Cells | | Ciliated Cells | | | |
| Early Response FOXJ1 high | CD164 | Early Response FOXJ1 high | PRRG4 | HOPX high Squamous Cells | GNG5 |
| Ciliated Cells | | Ciliated Cells | | | |
| Early Response FOXJ1 high | TUBA4B | Early Response FOXJ1 high | TBC1D8 | HOPX high Squamous Cells | PDLIM2 |
| Ciliated Cells | | Ciliated Cells | | | |
| Early Response FOXJ1 high | METTL7A | Early Response FOXJ1 high | PPP1R2 | HOPX high Squamous Cells | AKIRIN2 |
| Ciliated Cells | | Ciliated Cells | | | |
| Early Response FOXJ1 high | AZIN1 | Early Response FOXJ1 high | SKP1 | HOPX high Squamous Cells | PLEKHM1 |
| Ciliated Cells | | Ciliated Cells | | | |
| Early Response FOXJ1 high | DNAJB2 | Early Response FOXJ1 high | TMED4 | HOPX high Squamous Cells | NECAP2 |
| Ciliated Cells | | Ciliated Cells | | | |
| Early Response FOXJ1 high | GSTA2 | Early Response FOXJ1 high | PAIP2 | HOPX high Squamous Cells | GRHL3 |
| Ciliated Cells | | Ciliated Cells | | | |
| Early Response FOXJ1 high | TTC25 | Early Response FOXJ1 high | SCAND1 | HOPX high Squamous Cells | AL161645.1 |
| Ciliated Cells | | Ciliated Cells | | | |
| Early Response FOXJ1 high | PPIL6 | Early Response FOXJ1 high | VGLL4 | HOPX high Squamous Cells | ANO8 |
| Ciliated Cells | | Ciliated Cells | | | |
| Early Response FOXJ1 high | HDGF | Early Response FOXJ1 high | EXPH5 | HOPX high Squamous Cells | POLR2J3 |
| Ciliated Cells | | Ciliated Cells | | | |
| Early Response FOXJ1 high | CABCOCO1 | Early Response FOXJ1 high | SUGT1 | HOPX high Squamous Cells | NAPA |
| Ciliated Cells | | Ciliated Cells | | | |
| Early Response FOXJ1 high | PLTP | Early Response FOXJ1 high | PNRC1 | HOPX high Squamous Cells | TMC6 |
| Ciliated Cells | | Ciliated Cells | | | |
| Early Response FOXJ1 high | GSTA1 | Early Response FOXJ1 high | FNDC3A | HOPX high Squamous Cells | POLD4 |
| Ciliated Cells | | Ciliated Cells | | | |
| Early Response FOXJ1 high | DRC1 | Early Response FOXJ1 high | MAP3K13 | HOPX high Squamous Cells | ZSWIM4 |
| Ciliated Cells | | Ciliated Cells | | | |
| Early Response FOXJ1 high | TMEM231 | Early Response FOXJ1 high | CTSD | HOPX high Squamous Cells | STX7 |
| Ciliated Cells | | Ciliated Cells | | | |
| Early Response FOXJ1 high | EFHB | Early Response FOXJ1 high | TOB1 | HOPX high Squamous Cells | BMP1 |
| Ciliated Cells | | Ciliated Cells | | | |
| Early Response FOXJ1 high | PPP1R15A | Early Response FOXJ1 high | CDC42 | HOPX high Squamous Cells | PIEZO1 |
| Ciliated Cells | | Ciliated Cells | | | |
| Early Response FOXJ1 high | EFHC2 | Early Response FOXJ1 high | GNB2 | HOPX high Squamous Cells | PRKCH |
| Ciliated Cells | | Ciliated Cells | | | |
| Early Response FOXJ1 high | CLIC6 | Early Response FOXJ1 high | CCPG1 | HOPX high Squamous Cells | FURIN |
| Ciliated Cells | | Ciliated Cells | | | |
| Early Response FOXJ1 high | DYDC2 | Early Response FOXJ1 high | TRAP1 | HOPX high Squamous Cells | ARPC5L |
| Ciliated Cells | | Ciliated Cells | | | |
| Early Response FOXJ1 high | RSPH9 | Early Response FOXJ1 high | LRP10 | HOPX high Squamous Cells | TRIM38 |
| Ciliated Cells | | Ciliated Cells | | | |
| Early Response FOXJ1 high | MGLL | Early Response FOXJ1 high | C2CD5 | HOPX high Squamous Cells | SLC2A4RG |
| Ciliated Cells | | Ciliated Cells | | | |
| Early Response FOXJ1 high | TNFRSF19 | Early Response FOXJ1 high | CEBPB | HOPX high Squamous Cells | RASA2 |
| Ciliated Cells | | Ciliated Cells | | | |
| Early Response FOXJ1 high | MORN2 | Early Response FOXJ1 high | SUDS3 | HOPX high Squamous Cells | MRPL33 |
| Ciliated Cells | | Ciliated Cells | | | |
| Early Response FOXJ1 high | LAMC2 | Early Response FOXJ1 high | UBE2Q1 | HOPX high Squamous Cells | SH3TC2 |
| Ciliated Cells | | Ciliated Cells | | | |
| Early Response FOXJ1 high | PEBP1 | Early Response FOXJ1 high | TRIM8 | HOPX high Squamous Cells | CLDND1 |
| Ciliated Cells | | Ciliated Cells | | | |
| Early Response FOXJ1 high | PRR29 | Early Response FOXJ1 high | USP7 | HOPX high Squamous Cells | ZYX |
| Ciliated Cells | | Ciliated Cells | | | |
| Early Response FOXJ1 high | RUVBL1 | Early Response FOXJ1 high | KIAA0556 | HOPX high Squamous Cells | PPP3R1 |
| Ciliated Cells | | Ciliated Cells | | | |
| Early Response FOXJ1 high | CYB561 | Early Response FOXJ1 high | ATP6V1F | HOPX high Squamous Cells | KLK11 |
| Ciliated Cells | | Ciliated Cells | | | |
| Early Response FOXJ1 high | CCDC33 | Early Response FOXJ1 high | SNX2 | HOPX high Squamous Cells | RAB21 |
| Ciliated Cells | | Ciliated Cells | | | |
| Early Response FOXJ1 high | RCAN3 | Early Response FOXJ1 high | TCEANC2 | HOPX high Squamous Cells | AD000090.1 |
| Ciliated Cells | | Ciliated Cells | | | |
| Early Response FOXJ1 high | C9orf116 | Early Response FOXJ1 high | CSNK1D | HOPX high Squamous Cells | AC015712.2 |
| Ciliated Cells | | Ciliated Cells | | | |
| Early Response FOXJ1 high | FAM81B | Early Response FOXJ1 high | CEP162 | HOPX high Squamous Cells | MALL |
| Ciliated Cells | | Ciliated Cells | | | |
| Early Response FOXJ1 high | SOD1 | Early Response FOXJ1 high | ZBTB7A | HOPX high Squamous Cells | ZFAND2B |
| Ciliated Cells | | Ciliated Cells | | | |
| Early Response FOXJ1 high | C2orf40 | Early Response FOXJ1 high | RASAL2 | HOPX high Squamous Cells | TAX1BP3 |
| Ciliated Cells | | Ciliated Cells | | | |
| Early Response FOXJ1 high | CAPSL | Early Response FOXJ1 high | SRP9 | HOPX high Squamous Cells | RHBDF1 |
| Ciliated Cells | | Ciliated Cells | | | |
| Early Response FOXJ1 high | CCDC17 | Early Response FOXJ1 high | RUNX1 | HOPX high Squamous Cells | UNC13D |
| Ciliated Cells | | Ciliated Cells | | | |
| Early Response FOXJ1 high | TSPAN3 | Early Response FOXJ1 high | LARS | HOPX high Squamous Cells | FBLIM1 |
| Ciliated Cells | | Ciliated Cells | | | |
| Early Response FOXJ1 high | CYB5D1 | Early Response FOXJ1 high | MRPS21 | HOPX high Squamous Cells | PLEKHG2 |
| Ciliated Cells | | Ciliated Cells | | | |
| Early Response FOXJ1 high | PDK4 | Early Response FOXJ1 high | GDE1 | HOPX high Squamous Cells | GABARAP |
| Ciliated Cells | | Ciliated Cells | | | |
| Early Response FOXJ1 high | ALDH1A1 | Early Response FOXJ1 high | CHURC1 | HOPX high Squamous Cells | EHMT2 |
| Ciliated Cells | | Ciliated Cells | | | |
| Early Response FOXJ1 high | DNPH1 | Early Response FOXJ1 high | COPB1 | HOPX high Squamous Cells | A4GALT |
| Ciliated Cells | | Ciliated Cells | | | |
| Early Response FOXJ1 high | SLC22A4 | Early Response FOXJ1 high | KIF5B | HOPX high Squamous Cells | RAB5C |
| Ciliated Cells | | Ciliated Cells | | | |
| Early Response FOXJ1 high | CALM1 | Early Response FOXJ1 high | LRRC74B | HOPX high Squamous Cells | GAK |
| Ciliated Cells | | Ciliated Cells | | | |
| Early Response FOXJ1 high | IQCD | Early Response FOXJ1 high | ACAT1 | HOPX high Squamous Cells | GRK2 |
| Ciliated Cells | | Ciliated Cells | | | |
| Early Response FOXJ1 high | MAP3K19 | Early Response FOXJ1 high | SSB | HOPX high Squamous Cells | YIPF4 |
| Ciliated Cells | | Ciliated Cells | | | |
| Early Response FOXJ1 high | IQCG | Early Response FOXJ1 high | ZFR | HOPX high Squamous Cells | UBA6 |
| Ciliated Cells | | Ciliated Cells | | | |
| Early Response FOXJ1 high | CFAP52 | Early Response FOXJ1 high | RAB1A | HOPX high Squamous Cells | ARL6IP1 |
| Ciliated Cells | | Ciliated Cells | | | |
| Early Response FOXJ1 high | MFSD6 | Early Response FOXJ1 high | FAM102A | HOPX high Squamous Cells | CHMP5 |
| Ciliated Cells | | Ciliated Cells | | | |
| Early Response FOXJ1 high | PDLIM1 | Early Response FOXJ1 high | CDV3 | Interferon Responsive Ciliated | IFI6 |
| Ciliated Cells | | Ciliated Cells | | Cells | |
| Early Response FOXJ1 high | OSCP1 | Early Response FOXJ1 high | AC013470.2 | Interferon Responsive Ciliated | ISG15 |
| Ciliated Cells | | Ciliated Cells | | Cells | |
| Early Response FOXJ1 high | AL357093.2 | Early Response FOXJ1 high | AKR1C3 | Interferon Responsive Ciliated | IFIT1 |
| Ciliated Cells | | Ciliated Cells | | Cells | |
| Early Response FOXJ1 high | ATP5IF1 | Early Response FOXJ1 high | PSMC5 | Interferon Responsive Ciliated | IFI27 |
| Ciliated Cells | | Ciliated Cells | | Cells | |
| Early Response FOXJ1 high | DNAJB1 | Early Response FOXJ1 high | PSMC3 | Interferon Responsive Ciliated | IFITM3 |
| Ciliated Cells | | Ciliated Cells | | Cells | |
| Early Response FOXJ1 high | CMTM4 | Early Response FOXJ1 high | GSTO1 | Interferon Responsive Ciliated | IFIT3 |
| Ciliated Cells | | Ciliated Cells | | Cells | |
| Early Response FOXJ1 high | CD24 | Early Response FOXJ1 high | SIN3A | Interferon Responsive Ciliated | TUBB4B |
| Ciliated Cells | | Ciliated Cells | | Cells | |
| Early Response FOXJ1 high | EFCAB1 | Early Response FOXJ1 high | CEP89 | Interferon Responsive Ciliated | TUBA1A |
| Ciliated Cells | | Ciliated Cells | | Cells | |
| Early Response FOXJ1 high | RSPH3 | Early Response FOXJ1 high | NME7 | Interferon Responsive Ciliated | IFI44L |
| Ciliated Cells | | Ciliated Cells | | Cells | |
| Early Response FOXJ1 high | ANKUB1 | Early Response FOXJ1 high | BBIP1 | Interferon Responsive Ciliated | MX2 |
| Ciliated Cells | | Ciliated Cells | | Cells | |
| Early Response FOXJ1 high | UBB | Early Response FOXJ1 high | KPNA4 | Interferon Responsive Ciliated | SCO2 |
| Ciliated Cells | | Ciliated Cells | | Cells | |
| Early Response FOXJ1 high | CLMN | Early Response FOXJ1 high | SELENOP | Interferon Responsive Ciliated | CAPS |
| Ciliated Cells | | Ciliated Cells | | Cells | |
| Early Response FOXJ1 high | PPOX | Early Response FOXJ1 high | RER1 | Interferon Responsive Ciliated | TYMP |
| Ciliated Cells | | Ciliated Cells | | Cells | |
| Early Response FOXJ1 high | GAS2L2 | Early Response FOXJ1 high | SNU13 | Interferon Responsive Ciliated | C20orf85 |
| Ciliated Cells | | Ciliated Cells | | Cells | |
| Early Response FOXJ1 high | CCDC96 | Early Response FOXJ1 high | PDAP1 | Interferon Responsive Ciliated | IFITM1 |
| Ciliated Cells | | Ciliated Cells | | Cells | |
| Early Response FOXJ1 high | IFT46 | Early Response FOXJ1 high | IFT81 | Interferon Responsive Ciliated | OAS2 |
| Ciliated Cells | | Ciliated Cells | | Cells | |
| Early Response FOXJ1 high | SLC23A1 | Early Response FOXJ1 high | KIF27 | Interferon Responsive Ciliated | RRAD |
| Ciliated Cells | | Ciliated Cells | | Cells | |
| Early Response FOXJ1 high | FAM174A | Early Response FOXJ1 high | DUOX1 | Interferon Responsive Ciliated | C9orf24 |
| Ciliated Cells | | Ciliated Cells | | Cells | |
| Early Response FOXJ1 high | SELENOW | Early Response FOXJ1 high | GOLPH3 | Interferon Responsive Ciliated | LAP3 |
| Ciliated Cells | | Ciliated Cells | | Cells | |
| Early Response FOXJ1 high | RUVBL2 | Early Response FOXJ1 high | RAF1 | Interferon Responsive Ciliated | XAF1 |
| Ciliated Cells | | Ciliated Cells | | Cells | |
| Early Response FOXJ1 high | CHST6 | Early Response FOXJ1 high | TALDO1 | Interferon Responsive Ciliated | RSPH1 |
| Ciliated Cells | | Ciliated Cells | | Cells | |
| Early Response FOXJ1 high | MORN5 | Early Response FOXJ1 high | SPATA33 | Interferon Responsive Ciliated | OMG |
| Ciliated Cells | | Ciliated Cells | | Cells | |
| Early Response FOXJ1 high | P4HTM | Early Response FOXJ1 high | ATF6 | Interferon Responsive Ciliated | TSPAN1 |
| Ciliated Cells | | Ciliated Cells | | Cells | |
| Early Response FOXJ1 high | ENKUR | Early Response FOXJ1 high | NAA15 | Interferon Responsive Ciliated | CES1 |
| Ciliated Cells | | Ciliated Cells | | Cells | |
| Early Response FOXJ1 high | ECT2L | Early Response FOXJ1 high | COX411 | Interferon Responsive Ciliated | PSENEN |
| Ciliated Cells | | Ciliated Cells | | Cells | |
| Early Response FOXJ1 high | CC2D2A | Early Response FOXJ1 high | FARP1 | Interferon Responsive Ciliated | GSTA1 |
| Ciliated Cells | | Ciliated Cells | | Cells | |
| Early Response FOXJ1 high | ENAH | Early Response FOXJ1 high | THADA | Interferon Responsive Ciliated | C9orf116 |
| Ciliated Cells | | Ciliated Cells | | Cells | |
| Early Response FOXJ1 high | CCDC190 | Early Response FOXJ1 high | NFATC3 | Interferon Responsive Ciliated | ODF3B |
| Ciliated Cells | | Ciliated Cells | | Cells | |
| Early Response FOXJ1 high | SLC27A2 | Early Response FOXJ1 high | EXOC4 | Interferon Responsive Ciliated | ZMYND10 |
| Ciliated Cells | | Ciliated Cells | | Cells | |
| Early Response FOXJ1 high | KLF6 | Early Response FOXJ1 high | MRPS35 | Interferon Responsive Ciliated | SNTN |
| Ciliated Cells | | Ciliated Cells | | Cells | |
| Early Response FOXJ1 high | ENPP4 | Early Response FOXJ1 high | CNOT6 | Interferon Responsive Ciliated | C5orf49 |
| Ciliated Cells | | Ciliated Cells | | Cells | |
| Early Response FOXJ1 high | ARMC3 | Early Response FOXJ1 high | CYTH2 | Interferon Responsive Ciliated | AC007906.2 |
| Ciliated Cells | | Ciliated Cells | | Cells | |
| Early Response FOXJ1 high | CYP2S1 | Early Response FOXJ1 high | PLCB4 | Interferon Responsive Ciliated | BASP1 |
| Ciliated Cells | | Ciliated Cells | | Cells | |
| Early Response FOXJ1 high | HSPA8 | Early Response FOXJ1 high | COL21A1 | Interferon Responsive Ciliated | DNALI1 |
| Ciliated Cells | | Ciliated Cells | | Cells | |
| Early Response FOXJ1 high | CFAP45 | Early Response FOXJ1 high | ARMH1 | Interferon Responsive Ciliated | FAM92B |
| Ciliated Cells | | Ciliated Cells | | Cells | |
| Early Response FOXJ1 high | LRRC46 | Early Response Secretory Cells | ATF3 | Interferon Responsive Ciliated | LRRC10B |
| Ciliated Cells | | | | Cells | |
| Early Response FOXJ1 high | RFX3 | Early Response Secretory Cells | VMO1 | Interferon Responsive Ciliated | NUDC |
| Ciliated Cells | | | | Cells | |
| Early Response FOXJ1 high | PLAC8 | Early Response Secretory Cells | EGR1 | Interferon Responsive Ciliated | SPAG6 |
| Ciliated Cells | | | | Cells | |
| Early Response FOXJ1 high | TMEM123 | Early Response Secretory Cells | GDF15 | Interferon Responsive Ciliated | MORN2 |
| Ciliated Cells | | | | Cells | |
| Early Response FOXJ1 high | RHOU | Early Response Secretory Cells | BPIFB1 | Interferon Responsive Ciliated | PRDX5 |
| Ciliated Cells | | | | Cells | |
| Early Response FOXJ1 high | ERBB4 | Early Response Secretory Cells | FOS | Interferon Responsive Ciliated | STOML3 |
| Ciliated Cells | | | | Cells | |
| Early Response FOXJ1 high | STOX1 | Early Response Secretory Cells | BHLHE40 | Interferon Responsive Ciliated | C1orf194 |
| Ciliated Cells | | | | Cells | |
| Early Response FOXJ1 high | PSENEN | Early Response Secretory Cells | FOSB | Interferon Responsive Ciliated | TUBA4B |
| Ciliated Cells | | | | Cells | |
| Early Response FOXJ1 high | CCDC69 | Early Response Secretory Cells | JUN | Interferon Responsive Ciliated | C11orf88 |
| Ciliated Cells | | | | Cells | |
| Early Response FOXJ1 high | TRAF3IP1 | Early Response Secretory Cells | MAFF | Interferon Responsive Ciliated | HRASLS2 |
| Ciliated Cells | | | | Cells | |
| Early Response FOXJ1 high | SMYD2 | Early Response Secretory Cells | ZFP36 | Interferon Responsive Ciliated | PIFO |
| Ciliated Cells | | | | Cells | |
| Early Response FOXJ1 high | NELL2 | Early Response Secretory Cells | HSPA5 | Interferon Responsive Ciliated | FAM81B |
| Ciliated Cells | | | | Cells | |
| Early Response FOXJ1 high | MAGED2 | Early Response Secretory Cells | EPAS1 | Interferon Responsive Ciliated | FAM183A |
| Ciliated Cells | | | | Cells | |
| Early Response FOXJ1 high | STRBP | Early Response Secretory Cells | SOCS3 | Interferon Responsive Ciliated | CCDC17 |
| Ciliated Cells | | | | Cells | |
| Early Response FOXJ1 high | C9orf135 | Early Response Secretory Cells | KLF4 | Interferon Responsive Ciliated | CCDC114 |
| Ciliated Cells | | | | Cells | |
| Early Response FOXJ1 high | NEK11 | Early Response Secretory Cells | SLPI | Interferon Responsive Ciliated | LRRC46 |
| Ciliated Cells | | | | Cells | |
| Early Response FOXJ1 high | SHROOM3 | Early Response Secretory Cells | DNAJB1 | Interferon Responsive Ciliated | IK |
| Ciliated Cells | | | | Cells | |
| Early Response FOXJ1 high | PTPRN2 | Early Response Secretory Cells | NR4A1 | Interferon Responsive Ciliated | FOXJ1 |
| Ciliated Cells | | | | Cells | |
| Early Response FOXJ1 high | SEC14L3 | Early Response Secretory Cells | CLDN4 | Interferon Responsive Ciliated | TNFAIP8L1 |
| Ciliated Cells | | | | Cells | |
| Early Response FOXJ1 high | GNAS | Early Response Secretory Cells | WFDC2 | Interferon Responsive Ciliated | FAM229B |
| Ciliated Cells | | | | Cells | |
| Early Response FOXJ1 high | CCDC65 | Early Response Secretory Cells | CYP2F1 | Interferon Responsive Ciliated | UBXN10 |
| Ciliated Cells | | | | Cells | |
| Early Response FOXJ1 high | AC013264.1 | Early Response Secretory Cells | ID1 | Interferon Responsive Ciliated | CETN2 |
| Ciliated Cells | | | | Cells | |
| Early Response FOXJ1 high | ATF3 | Early Response Secretory Cells | PRSS23 | Interferon Responsive Ciliated | C9orf135 |
| Ciliated Cells | | | | Cells | |
| Early Response FOXJ1 high | PTPRF | Early Response Secretory Cells | ANPEP | Interferon Responsive Ciliated | CFAP126 |
| Ciliated Cells | | | | Cells | |
| Early Response FOXJ1 high | TUSC3 | Early Response Secretory Cells | MSLN | Interferon Responsive Ciliated | LRRC23 |
| Ciliated Cells | | | | Cells | |
| Early Response FOXJ1 high | IER5 | Early Response Secretory Cells | FAM107A | Interferon Responsive Ciliated | LDLRAD1 |
| Ciliated Cells | | | | Cells | |
| Early Response FOXJ1 high | ANKRD66 | Early Response Secretory Cells | FER1L6 | Interferon Responsive Ciliated | AL357093.2 |
| Ciliated Cells | | | | Cells | |
| Early Response FOXJ1 high | MPC2 | Early Response Secretory Cells | KRT7 | Interferon Responsive Ciliated | ENKUR |
| Ciliated Cells | | | | Cells | |
| Early Response FOXJ1 high | WLS | Early Response Secretory Cells | LYPD2 | Interferon Responsive Ciliated | ROPN1L |
| Ciliated Cells | | | | Cells | |
| Early Response FOXJ1 high | GCLC | Early Response Secretory Cells | RNF152 | Interferon Responsive Ciliated | IFT57 |
| Ciliated Cells | | | | Cells | |
| Early Response FOXJ1 high | CCDC81 | Early Response Secretory Cells | KCNK5 | Interferon Responsive Ciliated | MS4A8 |
| Ciliated Cells | | | | Cells | |
| Early Response FOXJ1 high | ADH7 | Early Response Secretory Cells | PI3 | Interferon Responsive Ciliated | RSPH4A |
| Ciliated Cells | | | | Cells | |
| Early Response FOXJ1 high | KIF21A | FOXJ1 high Ciliated Cells | CAPS | Interferon Responsive Ciliated | BAIAP3 |
| Ciliated Cells | | | | Cells | |
| Early Response FOXJ1 high | DNAL1 | FOXJ1 high Ciliated Cells | C20orf85 | Interferon Responsive Ciliated | SPA17 |
| Ciliated Cells | | | | Cells | |
| Early Response FOXJ1 high | HSD17B13 | FOXJ1 high Ciliated Cells | TPPP3 | Interferon Responsive Ciliated | NUCB2 |
| Ciliated Cells | | | | Cells | |
| Early Response FOXJ1 high | SPA17 | FOXJ1 high Ciliated Cells | RSPH1 | Interferon Responsive Ciliated | UFC1 |
| Ciliated Cells | | | | Cells | |
| Early Response FOXJ1 high | LRRC4 | FOXJ1 high Ciliated Cells | IGFBP7 | Interferon Responsive Ciliated | PKIG |
| Ciliated Cells | | | | Cells | |
| Early Response FOXJ1 high | PLEKHB1 | FOXJ1 high Ciliated Cells | TMEM190 | Interferon Responsive Ciliated | CAPSL |
| Ciliated Cells | | | | Cells | |
| Early Response FOXJ1 high | DYNLT1 | FOXJ1 high Ciliated Cells | CYP4B1 | Interferon Responsive Ciliated | WDR54 |
| Ciliated Cells | | | | Cells | |
| Early Response FOXJ1 high | BTBD3 | FOXJ1 high Ciliated Cells | AC007906.2 | Interferon Responsive Ciliated | MORN5 |
| Ciliated Cells | | | | Cells | |
| Early Response FOXJ1 high | PLEKHG7 | FOXJ1 high Ciliated Cells | FAM216B | Interferon Responsive Ciliated | CFAP100 |
| Ciliated Cells | | | | Cells | |
| Early Response FOXJ1 high | PRKAR1A | FOXJ1 high Ciliated Cells | C9orf24 | Interferon Responsive Ciliated | CCDC153 |
| Ciliated Cells | | | | Cells | |
| Early Response FOXJ1 high | SQLE | FOXJ1 high Ciliated Cells | LDLRAD1 | Interferon Responsive Ciliated | SPEF1 |
| Ciliated Cells | | | | Cells | |
| Early Response FOXJ1 high | C1orf194 | FOXJ1 high Ciliated Cells | SPAG6 | Interferon Responsive Ciliated | TEKT1 |
| Ciliated Cells | | | | Cells | |
| Early Response FOXJ1 high | LRIG1 | FOXJ1 high Ciliated Cells | CCDC170 | Interferon Responsive Ciliated | DRC3 |
| Ciliated Cells | | | | Cells | |
| Early Response FOXJ1 high | MLF1 | FOXJ1 high Ciliated Cells | CES1 | Interferon Responsive Ciliated | CCDC170 |
| Ciliated Cells | | | | Cells | |
| Early Response FOXJ1 high | HACD4 | FOXJ1 high Ciliated Cells | ZMYND10 | Interferon Responsive Ciliated | PRR29 |
| Ciliated Cells | | | | Cells | |
| Early Response FOXJ1 high | HHLA2 | FOXJ1 high Ciliated Cells | CD59 | Interferon Responsive Ciliated | FANK1 |
| Ciliated Cells | | | | Cells | |
| Early Response FOXJ1 high | ARMC4 | FOXJ1 high Ciliated Cells | UBXN10 | Interferon Responsive Ciliated | CFAP45 |
| Ciliated Cells | | | | Cells | |
| Early Response FOXJ1 high | MYCBP | FOXJ1 high Ciliated Cells | TUBA1A | Interferon Responsive Ciliated | DRC1 |
| Ciliated Cells | | | | Cells | |
| Early Response FOXJ1 high | KCTD12 | FOXJ1 high Ciliated Cells | ALDH3B1 | Interferon Responsive Ciliated | TTC25 |
| Ciliated Cells | | | | Cells | |
| Early Response FOXJ1 high | PERP | FOXJ1 high Ciliated Cells | CCDC17 | Interferon Responsive Ciliated | MAP1A |
| Ciliated Cells | | | | Cells | |
| Early Response FOXJ1 high | GNS | FOXJ1 high Ciliated Cells | PRDX5 | Interferon Responsive Ciliated | CFAP157 |
| Ciliated Cells | | | | Cells | |
| Early Response FOXJ1 high | MS4A8 | FOXJ1 high Ciliated Cells | ERICH3 | Interferon Responsive Ciliated | IFT22 |
| Ciliated Cells | | | | Cells | |
| Early Response FOXJ1 high | DDIT4 | FOXJ1 high Ciliated Cells | CDHR3 | Interferon Responsive Ciliated | CCDC65 |
| Ciliated Cells | | | | Cells | |
| Early Response FOXJ1 high | B3GNT5 | FOXJ1 high Ciliated Cells | RSPH4A | Interferon Responsive Ciliated | RSPH9 |
| Ciliated Cells | | | | Cells | |
| Early Response FOXJ1 high | GPR162 | FOXJ1 high Ciliated Cells | SNTN | Interferon Responsive Ciliated | FAM216B |
| Ciliated Cells | | | | Cells | |
| Early Response FOXJ1 high | TUFM | FOXJ1 high Ciliated Cells | NWD1 | Interferon Responsive Ciliated | SSBP4 |
| Ciliated Cells | | | | Cells | |
| Early Response FOXJ1 high | C12orf75 | FOXJ1 high Ciliated Cells | BASP1 | Interferon Responsive Ciliated | ERICH3 |
| Ciliated Cells | | | | Cells | |
| Early Response FOXJ1 high | GADD45G | FOXJ1 high Ciliated Cells | C5orf49 | Interferon Responsive Ciliated | CCDC113 |
| Ciliated Cells | | | | Cells | |
| Early Response FOXJ1 high | HMGN3 | FOXJ1 high Ciliated Cells | SPATA18 | Interferon Responsive Ciliated | CFAP73 |
| Ciliated Cells | | | | Cells | |
| Early Response FOXJ1 high | OMG | FOXJ1 high Ciliated Cells | PIFO | Interferon Responsive Ciliated | RUVBL1 |
| Ciliated Cells | | | | Cells | |
| Early Response FOXJ1 high | CFAP221 | FOXJ1 high Ciliated Cells | CKB | Interferon Responsive Ciliated | CCDC33 |
| Ciliated Cells | | | | Cells | |
| Early Response FOXJ1 high | DIAPH2 | FOXJ1 high Ciliated Cells | CETN2 | Interferon Responsive Ciliated | HAGHL |
| Ciliated Cells | | | | Cells | |
| Early Response FOXJ1 high | TSC22D1 | FOXJ1 high Ciliated Cells | MAP6 | Interferon Responsive Ciliated | IGFBP2 |
| Ciliated Cells | | | | Cells | |
| Early Response FOXJ1 high | ERGIC3 | FOXJ1 high Ciliated Cells | WDR78 | Interferon Responsive Ciliated | WDR90 |
| Ciliated Cells | | | | Cells | |
| Early Response FOXJ1 high | LRTOMT | FOXJ1 high Ciliated Cells | MAP1A | Interferon Responsive Ciliated | CCDC189 |
| Ciliated Cells | | | | Cells | |
| Early Response FOXJ1 high | WDR34 | FOXJ1 high Ciliated Cells | DNAH5 | Interferon Responsive Ciliated | CDHR4 |
| Ciliated Cells | | | | Cells | |
| Early Response FOXJ1 high | WRB | FOXJ1 high Ciliated Cells | CCDC113 | Interferon Responsive Ciliated | SPATA18 |
| Ciliated Cells | | | | Cells | |
| Early Response FOXJ1 high | CFAP57 | FOXJ1 high Ciliated Cells | SEC14L3 | Interferon Responsive Ciliated | TMEM231 |
| Ciliated Cells | | | | Cells | |
| Early Response FOXJ1 high | IQCE | FOXJ1 high Ciliated Cells | IFT57 | Interferon Responsive Ciliated | KIF9 |
| Ciliated Cells | | | | Cells | |
| Early Response FOXJ1 high | SPTLC2 | FOXJ1 high Ciliated Cells | STOML3 | Interferon Responsive Ciliated | C11orf97 |
| Ciliated Cells | | | | Cells | |
| Early Response FOXJ1 high | RIPOR2 | FOXJ1 high Ciliated Cells | TUBB4B | Interferon Responsive Ciliated | MAPK15 |
| Ciliated Cells | | | | Cells | |
| Early Response FOXJ1 high | AHSA1 | FOXJ1 high Ciliated Cells | FAM92B | Interferon Responsive Ciliated | MAP3K19 |
| Ciliated Cells | | | | Cells | |
| Early Response FOXJ1 high | UCKL1-AS1 | FOXJ1 high Ciliated Cells | TEKT1 | Interferon Responsive Ciliated | ANKUB1 |
| Ciliated Cells | | | | Cells | |
| Early Response FOXJ1 high | SRGAP3-AS2 | FOXJ1 high Ciliated Cells | ROPN1L | Interferon Responsive Ciliated | C11orf16 |
| Ciliated Cells | | | | Cells | |
| Early Response FOXJ1 high | ARL3 | FOXJ1 high Ciliated Cells | SLC44A4 | Interferon Responsive Ciliated | PPOX |
| Ciliated Cells | | | | Cells | |
| Early Response FOXJ1 high | PSAP | FOXJ1 high Ciliated Cells | ENPP5 | Interferon Responsive Ciliated | IQCG |
| Ciliated Cells | | | | Cells | |
| Early Response FOXJ1 high | AGBL2 | FOXJ1 high Ciliated Cells | DNALI1 | Interferon Responsive Ciliated | DNAIZ |
| Ciliated Cells | | | | Cells | |
| Early Response FOXJ1 high | HSPA5 | FOXJ1 high Ciliated Cells | SAMHD1 | Interferon Responsive Ciliated | BBOF1 |
| Ciliated Cells | | | | Cells | |
| Early Response FOXJ1 high | IL5RA | FOXJ1 high Ciliated Cells | ALOX15 | Interferon Responsive Ciliated | CCDC146 |
| Ciliated Cells | | | | Cells | |
| Early Response FOXJ1 high | ORAI2 | FOXJ1 high Ciliated Cells | AK7 | Interferon Responsive Ciliated | RAB36 |
| Ciliated Cells | | | | Cells | |
| Early Response FOXJ1 high | C11orf16 | FOXJ1 high Ciliated Cells | SLC7A2 | Interferon Responsive Ciliated | PACRG |
| Ciliated Cells | | | | Cells | |
| Early Response FOXJ1 high | TOMM34 | FOXJ1 high Ciliated Cells | TNFAIP8L1 | Interferon Responsive Ciliated | NME5 |
| Ciliated Cells | | | | Cells | |
| Early Response FOXJ1 high | TPGS2 | FOXJ1 high Ciliated Cells | PRR29 | Interferon Responsive Ciliated | MNS1 |
| Ciliated Cells | | | | Cells | |
| Early Response FOXJ1 high | ATP5F1B | FOXJ1 high Ciliated Cells | UFC1 | Interferon Responsive Ciliated | EFHC1 |
| Ciliated Cells | | | | Cells | |
| Early Response FOXJ1 high | CES2 | FOXJ1 high Ciliated Cells | GSTA1 | Interferon Responsive Ciliated | CCDC190 |
| Ciliated Cells | | | | Cells | |
| Early Response FOXJ1 high | CFAP126 | FOXJ1 high Ciliated Cells | DNAJA4 | Interferon Responsive Ciliated | AK7 |
| Ciliated Cells | | | | Cells | |
| Early Response FOXJ1 high | GLB1L | FOXJ1 high Ciliated Cells | ENKUR | Interferon Responsive Ciliated | DNAAF3 |
| Ciliated Cells | | | | Cells | |
| Early Response FOXJ1 high | ATP9A | FOXJ1 high Ciliated Cells | DRC1 | Interferon Responsive Ciliated | DTHD1 |
| Ciliated Cells | | | | Cells | |
| Early Response FOXJ1 high | APOD | FOXJ1 high Ciliated Cells | AL357093.2 | Interferon Responsive Ciliated | EFHB |
| Ciliated Cells | | | | Cells | |
| Early Response FOXJ1 high | PRDX1 | FOXJ1 high Ciliated Cells | WDR54 | Interferon Responsive Ciliated | DNAAF1 |
| Ciliated Cells | | | | Cells | |
| Early Response FOXJ1 high | COPRS | FOXJ1 high Ciliated Cells | MORN2 | Interferon Responsive Ciliated | CC2D2A |
| Ciliated Cells | | | | Cells | |
| Early Response FOXJ1 high | DNAH5 | FOXJ1 high Ciliated Cells | TSPAN1 | Interferon Responsive Ciliated | C6orf118 |
| Ciliated Cells | | | | Cells | |
| Early Response FOXJ1 high | SCPEP1 | FOXJ1 high Ciliated Cells | LRRC23 | Interferon Responsive Ciliated | IFT172 |
| Ciliated Cells | | | | Cells | |
| Early Response FOXJ1 high | COPB2 | FOXJ1 high Ciliated Cells | EFHC1 | Interferon Responsive Ciliated | TTC29 |
| Ciliated Cells | | | | Cells | |
| Early Response FOXJ1 high | PRSS12 | FOXJ1 high Ciliated Cells | LRRC10B | Interferon Responsive Ciliated | ARMC3 |
| Ciliated Cells | | | | Cells | |
| Early Response FOXJ1 high | FAM229B | FOXJ1 high Ciliated Cells | DNAH9 | Interferon Responsive Ciliated | LRRC71 |
| Ciliated Cells | | | | Cells | |
| Early Response FOXJ1 high | ATXN7L3B | FOXJ1 high Ciliated Cells | EFCAB1 | Interferon Responsive Ciliated | CCDC78 |
| Ciliated Cells | | | | Cells | |
| Early Response FOXJ1 high | EFHC1 | FOXJ1 high Ciliated Cells | CIB1 | Interferon Responsive Ciliated | TEKT2 |
| Ciliated Cells | | | | Cells | |
| Early Response FOXJ1 high | TCTEX1D4 | FOXJ1 high Ciliated Cells | MS4A8 | Interferon Responsive Ciliated | DYDC2 |
| Ciliated Cells | | | | Cells | |
| Early Response FOXJ1 high | SPACA9 | FOXJ1 high Ciliated Cells | DRC3 | Interferon Responsive Ciliated | CFAP46 |
| Ciliated Cells | | | | Cells | |
| Early Response FOXJ1 high | KIAA1211L | FOXJ1 high Ciliated Cells | C2orf40 | Interferon Responsive Ciliated | DZIP3 |
| Ciliated Cells | | | | Cells | |
| Early Response FOXJ1 high | DDB1 | FOXJ1 high Ciliated Cells | CCDC33 | Interferon Responsive Ciliated | ANKRD66 |
| Ciliated Cells | | | | Cells | |
| Early Response FOXJ1 high | FANK1 | FOXJ1 high Ciliated Cells | RRAD | Interferon Responsive Ciliated | PPIL6 |
| Ciliated Cells | | | | Cells | |
| Early Response FOXJ1 high | ATP2A2 | FOXJ1 high Ciliated Cells | UCP2 | Interferon Responsive Ciliated | WDR78 |
| Ciliated Cells | | | | Cells | |
| Early Response FOXJ1 high | DAW1 | FOXJ1 high Ciliated Cells | LRRC46 | Interferon Responsive Ciliated | CCDC187 |
| Ciliated Cells | | | | Cells | |
| Early Response FOXJ1 high | APBB1 | FOXJ1 high Ciliated Cells | CFAP157 | Interferon Responsive Ciliated | SAXO2 |
| Ciliated Cells | | | | Cells | |
| Early Response FOXJ1 high | RNPEP | FOXJ1 high Ciliated Cells | CFAP45 | Interferon Responsive Ciliated | EFCAB1 |
| Ciliated Cells | | | | Cells | |
| Early Response FOXJ1 high | SLC20A2 | FOXJ1 high Ciliated Cells | PROM1 | Interferon Responsive Ciliated | STK33 |
| Ciliated Cells | | | | Cells | |
| Early Response FOXJ1 high | WDR38 | FOXJ1 high Ciliated Cells | IGFBP5 | Interferon Responsive Ciliated | CFAP65 |
| Ciliated Cells | | | | Cells | |
| Early Response FOXJ1 high | NAT14 | FOXJ1 high Ciliated Cells | BAIAP3 | Interferon Responsive Ciliated | CFAP53 |
| Ciliated Cells | | | | Cells | |
| Early Response FOXJ1 high | POLR21 | FOXJ1 high Ciliated Cells | IQCG | Interferon Responsive Ciliated | CCDC40 |
| Ciliated Cells | | | | Cells | |
| Early Response FOXJ1 high | MAP1B | FOXJ1 high Ciliated Cells | CAPSL | Interferon Responsive Ciliated | TSNAXIP1 |
| Ciliated Cells | | | | Cells | |
| Early Response FOXJ1 high | CAT | FOXJ1 high Ciliated Cells | DNPH1 | Interferon Responsive Ciliated | SPATA17 |
| Ciliated Cells | | | | Cells | |
| Early Response FOXJ1 high | C4orf3 | FOXJ1 high Ciliated Cells | VWA3B | Interferon Responsive Ciliated | TTC21A |
| Ciliated Cells | | | | Cells | |
| Early Response FOXJ1 high | ANXA2 | FOXJ1 high Ciliated Cells | ARMC3 | Interferon Responsive Ciliated | WDR66 |
| Ciliated Cells | | | | Cells | |
| Early Response FOXJ1 high | TRAK1 | FOXJ1 high Ciliated Cells | C11orf88 | Interferon Responsive Ciliated | CABCOCO1 |
| Ciliated Cells | | | | Cells | |
| Early Response FOXJ1 high | C11orf88 | FOXJ1 high Ciliated Cells | LRP11 | Interferon Responsive Ciliated | DLEC1 |
| Ciliated Cells | | | | Cells | |
| Early Response FOXJ1 high | STEAP3 | FOXJ1 high Ciliated Cells | P4HTM | Interferon Responsive Ciliated | DZIP1L |
| Ciliated Cells | | | | Cells | |
| Early Response FOXJ1 high | TXNRD1 | FOXJ1 high Ciliated Cells | PPOX | Interferon Responsive Ciliated | CFAP57 |
| Ciliated Cells | | | | Cells | |
| Early Response FOXJ1 high | GBP6 | FOXJ1 high Ciliated Cells | EFHB | Interferon Responsive Ciliated | NEK11 |
| Ciliated Cells | | | | Cells | |
| Early Response FOXJ1 high | ADM | FOXJ1 high Ciliated Cells | GSTA2 | Interferon Responsive Ciliated | NEK10 |
| Ciliated Cells | | | | Cells | |
| Early Response FOXJ1 high | DZIP3 | FOXJ1 high Ciliated Cells | IGFBP2 | Interferon Responsive Ciliated | DNAI1 |
| Ciliated Cells | | | | Cells | |
| Early Response FOXJ1 high | ID2 | FOXJ1 high Ciliated Cells | ATP5IF1 | Interferon Responsive Ciliated | CFAP52 |
| Ciliated Cells | | | | Cells | |
| Early Response FOXJ1 high | ACO2 | FOXJ1 high Ciliated Cells | TTC25 | Interferon Responsive Ciliated | CFAP300 |
| Ciliated Cells | | | | Cells | |
| Early Response FOXJ1 high | MAOB | FOXJ1 high Ciliated Cells | TCTEX1D4 | Interferon Responsive Ciliated | VWA3A |
| Ciliated Cells | | | | Cells | |
| Early Response FOXJ1 high | CCDC153 | FOXJ1 high Ciliated Cells | CDHR4 | Interferon Responsive Ciliated | RP1 |
| Ciliated Cells | | | | Cells | |
| Early Response FOXJ1 high | SRI | FOXJ1 high Ciliated Cells | CFAP100 | Interferon Responsive Ciliated | NEK5 |
| Ciliated Cells | | | | Cells | |
| Early Response FOXJ1 high | ICMT | FOXJ1 high Ciliated Cells | OMG | Interferon Responsive Ciliated | CASC1 |
| Ciliated Cells | | | | Cells | |
| Early Response FOXJ1 high | AP2B1 | FOXJ1 high Ciliated Cells | PSENEN | Interferon Responsive Ciliated | LRRIQ1 |
| Ciliated Cells | | | | Cells | |
| Early Response FOXJ1 high | WDR66 | FOXJ1 high Ciliated Cells | CC2D2A | Interferon Responsive Ciliated | DNAH9 |
| Ciliated Cells | | | | Cells | |
| Early Response FOXJ1 high | SRD5A2 | FOXJ1 high Ciliated Cells | C9orf116 | Interferon Responsive Ciliated | FHAD1 |
| Ciliated Cells | | | | Cells | |
| Early Response FOXJ1 high | PLEKHS1 | FOXJ1 high Ciliated Cells | FABP6 | Interferon Responsive Ciliated | MOK |
| Ciliated Cells | | | | Cells | |
| Early Response FOXJ1 high | BBOF1 | FOXJ1 high Ciliated Cells | CCDC190 | Interferon Responsive Ciliated | EFCAB12 |
| Ciliated Cells | | | | Cells | |
| Early Response FOXJ1 high | ABHD12B | FOXJ1 high Ciliated Cells | CHST9 | Interferon Responsive Ciliated | RFX3 |
| Ciliated Cells | | | | Cells | |
| Early Response FOXJ1 high | PSMB4 | FOXJ1 high Ciliated Cells | TSPAN6 | Interferon Responsive Ciliated | CCDC173 |
| Ciliated Cells | | | | Cells | |
| Early Response FOXJ1 high | IK | FOXJ1 high Ciliated Cells | SPEF1 | Interferon Responsive Ciliated | CFAP74 |
| Ciliated Cells | | | | Cells | |
| Early Response FOXJ1 high | C11orf97 | FOXJ1 high Ciliated Cells | CES4A | Interferon Responsive Ciliated | LRRC74B |
| Ciliated Cells | | | | Cells | |
| Early Response FOXJ1 high | KIF2A | FOXJ1 high Ciliated Cells | SAXO2 | Interferon Responsive Ciliated | CCDC39 |
| Ciliated Cells | | | | Cells | |
| Early Response FOXJ1 high | GLIPR2 | FOXJ1 high Ciliated Cells | SYNE1 | Interferon Responsive Ciliated | EFCAB10 |
| Ciliated Cells | | | | Cells | |
| Early Response FOXJ1 high | MTURN | FOXJ1 high Ciliated Cells | WDR66 | Interferon Responsive Ciliated | TSPAN19 |
| Ciliated Cells | | | | Cells | |
| Early Response FOXJ1 high | EPB41L4B | FOXJ1 high Ciliated Cells | PKIG | Interferon Responsive Ciliated | CFAP69 |
| Ciliated Cells | | | | Cells | |
| Early Response FOXJ1 high | NUCB2 | FOXJ1 high Ciliated Cells | DZIP3 | Interferon Responsive Ciliated | HYDIN |
| Ciliated Cells | | | | Cells | |
| Early Response FOXJ1 high | TCTN1 | FOXJ1 high Ciliated Cells | TUBA4B | Interferon Responsive Ciliated | CCDC180 |
| Ciliated Cells | | | | Cells | |
| Early Response FOXJ1 high | TMC4 | FOXJ1 high Ciliated Cells | NELL2 | Interferon Responsive Ciliated | NPHP1 |
| Ciliated Cells | | | | Cells | |
| Early Response FOXJ1 high | TPRG1L | FOXJ1 high Ciliated Cells | NUDC | Interferon Responsive Ciliated | WDR63 |
| Ciliated Cells | | | | Cells | |
| Early Response FOXJ1 high | PFN2 | FOXJ1 high Ciliated Cells | CFAP52 | Interferon Responsive Ciliated | CFAP43 |
| Ciliated Cells | | | | Cells | |
| Early Response FOXJ1 high | GALC | FOXJ1 high Ciliated Cells | C1orf194 | Interferon Responsive Ciliated | MAATS1 |
| Ciliated Cells | | | | Cells | |
| Early Response FOXJ1 high | ANKRD65 | FOXJ1 high Ciliated Cells | PACRG | Interferon Responsive Ciliated | ZBBX |
| Ciliated Cells | | | | Cells | |
| Early Response FOXJ1 high | SPTBN1 | FOXJ1 high Ciliated Cells | CCDC80 | Interferon Responsive Ciliated | CEP126 |
| Ciliated Cells | | | | Cells | |
| Early Response FOXJ1 high | CD38 | FOXJ1 high Ciliated Cells | KLHL6 | Interferon Responsive Ciliated | CFAP70 |
| Ciliated Cells | | | | Cells | |
| Early Response FOXJ1 high | RIBC1 | FOXJ1 high Ciliated Cells | KIF3B | Interferon Responsive Ciliated | DNAH5 |
| Ciliated Cells | | | | Cells | |
| Early Response FOXJ1 high | COQ4 | FOXJ1 high Ciliated Cells | FAM81B | Interferon Responsive Ciliated | DNAH2 |
| Ciliated Cells | | | | Cells | |
| Early Response FOXJ1 high | VPS35 | FOXJ1 high Ciliated Cells | MAP3K19 | Interferon Responsive Ciliated | SYNE1 |
| Ciliated Cells | | | | Cells | |
| Early Response FOXJ1 high | MID1IP1 | FOXJ1 high Ciliated Cells | MORN5 | Interferon Responsive Ciliated | FAM227A |
| Ciliated Cells | | | | Cells | |
| Early Response FOXJ1 high | ANKRD42 | FOXJ1 high Ciliated Cells | CDS1 | Interferon Responsive Ciliated | SPAG17 |
| Ciliated Cells | | | | Cells | |
| Early Response FOXJ1 high | EFCAB10 | FOXJ1 high Ciliated Cells | RUVBL1 | Interferon Responsive Ciliated | DNAH6 |
| Ciliated Cells | | | | Cells | |
| Early Response FOXJ1 high | TTC29 | FOXJ1 high Ciliated Cells | ZNF106 | Interferon Responsive Ciliated | SPEF2 |
| Ciliated Cells | | | | Cells | |
| Early Response FOXJ1 high | INHBB | FOXJ1 high Ciliated Cells | TMEM231 | Interferon Responsive Ciliated | DNAH3 |
| Ciliated Cells | | | | Cells | |
| Early Response FOXJ1 high | HIPK3 | FOXJ1 high Ciliated Cells | ECT2L | Interferon Responsive Ciliated | DNAH11 |
| Ciliated Cells | | | | Cells | |
| Early Response FOXJ1 high | ODF3B | FOXJ1 high Ciliated Cells | ANKUB1 | Interferon Responsive Ciliated | DNAAF4 |
| Ciliated Cells | | | | Cells | |
| Early Response FOXJ1 high | DTHD1 | FOXJ1 high Ciliated Cells | PPIL6 | Interferon Responsive Ciliated | KIAA2012 |
| Ciliated Cells | | | | Cells | |
| Early Response FOXJ1 high | ARHGAP18 | FOXJ1 high Ciliated Cells | DNAH10 | Interferon Responsive Ciliated | CFAP44 |
| Ciliated Cells | | | | Cells | |
| Early Response FOXJ1 high | EIF2AK1 | FOXJ1 high Ciliated Cells | CFAP57 | Interferon Responsive Ciliated | DNAH10 |
| Ciliated Cells | | | | Cells | |
| Early Response FOXJ1 high | DPCD | FOXJ1 high Ciliated Cells | SPA17 | Interferon Responsive Ciliated | DNAH12 |
| Ciliated Cells | | | | Cells | |
| Early Response FOXJ1 high | SPATA6 | FOXJ1 high Ciliated Cells | C6orf118 | Interferon Responsive Ciliated | DNAH7 |
| Ciliated Cells | | | | Cells | |
| Early Response FOXJ1 high | CFAP300 | FOXJ1 high Ciliated Cells | CCDC187 | Interferon Responsive Ciliated | CSPP1 |
| Ciliated Cells | | | | Cells | |
| Early Response FOXJ1 high | MTSS1 | FOXJ1 high Ciliated Cells | KIF21A | Ionocytes | ADGRF5 |
| Ciliated Cells | | | | | |
| Early Response FOXJ1 high | CITED2 | FOXJ1 high Ciliated Cells | CCDC65 | Ionocytes | STAP1 |
| Ciliated Cells | | | | | |
| Early Response FOXJ1 high | BBS4 | FOXJ1 high Ciliated Cells | CCDC153 | Ionocytes | CFTR |
| Ciliated Cells | | | | | |
| Early Response FOXJ1 high | FAM183A | FOXJ1 high Ciliated Cells | NQO1 | Ionocytes | RARRES2 |
| Ciliated Cells | | | | | |
| Early Response FOXJ1 high | TXNIP | FOXJ1 high Ciliated Cells | RUVBL2 | Ionocytes | SCNN1B |
| Ciliated Cells | | | | | |
| Early Response FOXJ1 high | ARFGEF3 | FOXJ1 high Ciliated Cells | FAM183A | Ionocytes | CLCNKB |
| Ciliated Cells | | | | | |
| Early Response FOXJ1 high | DRC3 | FOXJ1 high Ciliated Cells | RIBC1 | Ionocytes | ITPR2 |
| Ciliated Cells | | | | | |
| Early Response FOXJ1 high | LBH | FOXJ1 high Ciliated Cells | SLC27A2 | Ionocytes | ANK2 |
| Ciliated Cells | | | | | |
| Early Response FOXJ1 high | STK33 | FOXJ1 high Ciliated Cells | DTHD1 | Ionocytes | ITIH5 |
| Ciliated Cells | | | | | |
| Early Response FOXJ1 high | CCDC74A | FOXJ1 high Ciliated Cells | IK | Ionocytes | ASCL3 |
| Ciliated Cells | | | | | |
| Early Response FOXJ1 high | MAPK8IP1 | FOXJ1 high Ciliated Cells | EZR | Ionocytes | IGF1 |
| Ciliated Cells | | | | | |
| Early Response FOXJ1 high | RAB36 | FOXJ1 high Ciliated Cells | SARAF | Ionocytes | THBS1 |
| Ciliated Cells | | | | | |
| Early Response FOXJ1 high | GPX4 | FOXJ1 high Ciliated Cells | DYNLL1 | Ionocytes | TFCP2L1 |
| Ciliated Cells | | | | | |
| Early Response FOXJ1 high | NSUN7 | FOXJ1 high Ciliated Cells | IFT172 | Ionocytes | RNF152 |
| Ciliated Cells | | | | | |
| Early Response FOXJ1 high | ABI2 | FOXJ1 high Ciliated Cells | SLC22A4 | Ionocytes | ATP6V1A |
| Ciliated Cells | | | | | |
| Early Response FOXJ1 high | EIF4G2 | FOXJ1 high Ciliated Cells | ODF3B | Ionocytes | PPP1R12B |
| Ciliated Cells | | | | | |
| Early Response FOXJ1 high | SOX2 | FOXJ1 high Ciliated Cells | CCDC146 | Ionocytes | DST |
| Ciliated Cells | | | | | |
| Early Response FOXJ1 high | WFDC6 | FOXJ1 high Ciliated Cells | GAS2L2 | Ionocytes | PLCG2 |
| Ciliated Cells | | | | | |
| Early Response FOXJ1 high | EGLN3 | FOXJ1 high Ciliated Cells | ARMC4 | Ionocytes | DGKI |
| Ciliated Cells | | | | | |
| Early Response FOXJ1 high | ANKRD54 | FOXJ1 high Ciliated Cells | PLTP | Ionocytes | PDE1C |
| Ciliated Cells | | | | | |
| Early Response FOXJ1 high | SLFN13 | FOXJ1 high Ciliated Cells | LAMC2 | Ionocytes | LINC01187 |
| Ciliated Cells | | | | | |
| Early Response FOXJ1 high | STUB1 | FOXJ1 high Ciliated Cells | CYB561 | Ionocytes | HEPACAM2 |
| Ciliated Cells | | | | | |
| Early Response FOXJ1 high | RSPH14 | FOXJ1 high Ciliated Cells | DYNC2H1 | Ionocytes | BSND |
| Ciliated Cells | | | | | |
| Early Response FOXJ1 high | BAIAP3 | FOXJ1 high Ciliated Cells | DNAH2 | Ionocytes | SCNN1G |
| Ciliated Cells | | | | | |
| Early Response FOXJ1 high | FDXR | FOXJ1 high Ciliated Cells | C22orf15 | Ionocytes | FOXI1 |
| Ciliated Cells | | | | | |
| Early Response FOXJ1 high | ERICH5 | FOXJ1 high Ciliated Cells | METTL7A | Ionocytes | CLNK |
| Ciliated Cells | | | | | |
| Early Response FOXJ1 high | COL28A1 | FOXJ1 high Ciliated Cells | CLIC6 | Ionocytes | KIT |
| Ciliated Cells | | | | | |
| Early Response FOXJ1 high | SIX4 | FOXJ1 high Ciliated Cells | IFT140 | Ionocytes | HIPK2 |
| Ciliated Cells | | | | | |
| Early Response FOXJ1 high | SNTB1 | FOXJ1 high Ciliated Cells | SMYD2 | Ionocytes | SLC43A2 |
| Ciliated Cells | | | | | |
| Early Response FOXJ1 high | EFCAB11 | FOXJ1 high Ciliated Cells | MAP1B | Ionocytes | SSFA2 |
| Ciliated Cells | | | | | |
| Early Response FOXJ1 high | LZTFL1 | FOXJ1 high Ciliated Cells | IFT22 | Ionocytes | DMRT2 |
| Ciliated Cells | | | | | |
| Early Response FOXJ1 high | NEK5 | FOXJ1 high Ciliated Cells | HSD17B13 | Ionocytes | C1orf115 |
| Ciliated Cells | | | | | |
| Early Response FOXJ1 high | PROS1 | FOXJ1 high Ciliated Cells | HAGHL | Ionocytes | ATP6V1G3 |
| Ciliated Cells | | | | | |
| Early Response FOXJ1 high | ENKD1 | FOXJ1 high Ciliated Cells | EFHC2 | Ionocytes | LTBP2 |
| Ciliated Cells | | | | | |
| Early Response FOXJ1 high | PRR15 | FOXJ1 high Ciliated Cells | ZNF664 | Ionocytes | GOLM1 |
| Ciliated Cells | | | | | |
| Early Response FOXJ1 high | PGRMC1 | FOXJ1 high Ciliated Cells | CFAP53 | Ionocytes | APLP2 |
| Ciliated Cells | | | | | |
| Early Response FOXJ1 high | CDH1 | FOXJ1 high Ciliated Cells | FANK1 | Ionocytes | CLCNKA |
| Ciliated Cells | | | | | |
| Early Response FOXJ1 high | ZBED5-AS1 | FOXJ1 high Ciliated Cells | FOXJ1 | Ionocytes | SLC14A1 |
| Ciliated Cells | | | | | |
| Early Response FOXJ1 high | KLF2 | FOXJ1 high Ciliated Cells | IQCD | Ionocytes | FOXI2 |
| Ciliated Cells | | | | | |
| Early Response FOXJ1 high | CFAP73 | FOXJ1 high Ciliated Cells | KIAA1211L | Ionocytes | PTGER3 |
| Ciliated Cells | | | | | |
| Early Response FOXJ1 high | SECISBP2L | FOXJ1 high Ciliated Cells | TRAF3IP1 | Ionocytes | SCUBE2 |
| Ciliated Cells | | | | | |
| Early Response FOXJ1 high | NRAV | FOXJ1 high Ciliated Cells | RFX3 | Ionocytes | ATP6V0D2 |
| Ciliated Cells | | | | | |
| Early Response FOXJ1 high | SAP18 | FOXJ1 high Ciliated Cells | CFAP65 | Ionocytes | RCAN2 |
| Ciliated Cells | | | | | |
| Early Response FOXJ1 high | LRRC6 | FOXJ1 high Ciliated Cells | DNAAF1 | KRT24 KRT13 high | KRT4 |
| Ciliated Cells | | | | Secretory Cells | |
| Early Response FOXJ1 high | C6orf118 | FOXJ1 high Ciliated Cells | BEST4 | Mitotic Basal Cells | MKI67 |
| Ciliated Cells | | | | | |
| Early Response FOXJ1 high | HNRNPF | FOXJ1 high Ciliated Cells | RSPH9 | Mitotic Basal Cells | CENPF |
| Ciliated Cells | | | | | |
| Early Response FOXJ1 high | SSBP4 | FOXJ1 high Ciliated Cells | SRGAP3- | Mitotic Basal Cells | TOP2A |
| Ciliated Cells | | | AS2 | | |
| Early Response FOXJ1 high | SERPINB6 | FOXJ1 high Ciliated Cells | SSBP4 | MUC5AC high Goblet Cells | ANKRD36C |
| Ciliated Cells | | | | | |
| Early Response FOXJ1 high | TTC26 | FOXJ1 high Ciliated Cells | CCDC189 | MUC5AC high Goblet Cells | MALAT1 |
| Ciliated Cells | | | | | |
| Early Response FOXJ1 high | ATP6AP1 | FOXJ1 high Ciliated Cells | FAM229B | MUC5AC high Goblet Cells | NEAT1 |
| Ciliated Cells | | | | | |
| Early Response FOXJ1 high | SYTL3 | FOXJ1 high Ciliated Cells | HMGN3 | MUC5AC high Goblet Cells | ERN2 |
| Ciliated Cells | | | | | |
| Early Response FOXJ1 high | ZSCAN18 | FOXJ1 high Ciliated Cells | CD164 | MUC5AC high Goblet Cells | SRRM2 |
| Ciliated Cells | | | | | |
| Early Response FOXJ1 high | HSDL2 | FOXJ1 high Ciliated Cells | OSCP1 | SCGB1A1 high Goblet Cells | SCGB1A1 |
| Ciliated Cells | | | | | |
| Early Response FOXJ1 high | CYSTM1 | FOXJ1 high Ciliated Cells | NUCB2 | SCGB1A1 high Goblet Cells | TMEM213 |
| Ciliated Cells | | | | | |
| Early Response FOXJ1 high | SIX1 | FOXJ1 high Ciliated Cells | CFAP43 | SCGB1A1 high Goblet Cells | SLPI |
| Ciliated Cells | | | | | |
| Early Response FOXJ1 high | ANXA5 | FOXJ1 high Ciliated Cells | PLEKHB1 | SCGB1A1 high Goblet Cells | SERPINB3 |
| Ciliated Cells | | | | | |
| Early Response FOXJ1 high | CERKL | FOXJ1 high Ciliated Cells | TSPAN3 | SCGB1A1 high Goblet Cells | EPAS1 |
| Ciliated Cells | | | | | |
| Early Response FOXJ1 high | MUC15 | FOXJ1 high Ciliated Cells | RSPH3 | SCGB1A1 high Goblet Cells | PRSS23 |
| Ciliated Cells | | | | | |
| Early Response FOXJ1 high | USP2 | FOXJ1 high Ciliated Cells | BBOF1 | SERPINB11 high Secretory | SERPINB3 |
| Ciliated Cells | | | | Cells | |
| Early Response FOXJ1 high | SELENBP1 | FOXJ1 high Ciliated Cells | DYNLT1 | SERPINB11 high Secretory | TMSB4X |
| Ciliated Cells | | | | Cells | |
| Early Response FOXJ1 high | KLHDC9 | FOXJ1 high Ciliated Cells | CCDC114 | SERPINB11 high Secretory | AGR2 |
| Ciliated Cells | | | | Cells | |
| Early Response FOXJ1 high | F11R | FOXJ1 high Ciliated Cells | PTPRN2 | SERPINB11 high Secretory | RPS18 |
| Ciliated Cells | | | | Cells | |
| Early Response FOXJ1 high | OSBPL6 | FOXJ1 high Ciliated Cells | LINC01765 | SPRR2D high Squamous Cells | S100A8 |
| Ciliated Cells | | | | | |
| Early Response FOXJ1 high | PRRT3 | FOXJ1 high Ciliated Cells | COL28A1 | SPRR2D high Squamous Cells | SPRR2A |
| Ciliated Cells | | | | | |
| Early Response FOXJ1 high | SLC2A1 | FOXJ1 high Ciliated Cells | SOD1 | SPRR2D high Squamous Cells | SPRR1B |
| Ciliated Cells | | | | | |
| Early Response FOXJ1 high | ANKMY1 | FOXJ1 high Ciliated Cells | OSBPL6 | SPRR2D high Squamous Cells | KRT6A |
| Ciliated Cells | | | | | |
| Early Response FOXJ1 high | CDKL1 | FOXJ1 high Ciliated Cells | FAM174A | SPRR2D high Squamous Cells | SPRR2E |
| Ciliated Cells | | | | | |
| Early Response FOXJ1 high | CUTA | FOXJ1 high Ciliated Cells | C9orf135 | SPRR2D high Squamous Cells | S100A9 |
| Ciliated Cells | | | | | |
| Early Response FOXJ1 high | VCP | FOXJ1 high Ciliated Cells | MAPK15 | SPRR2D high Squamous Cells | SPRR2D |
| Ciliated Cells | | | | | |
| Early Response FOXJ1 high | DYNLRB2 | FOXJ1 high Ciliated Cells | DNAI1 | SPRR2D high Squamous Cells | SPRR3 |
| Ciliated Cells | | | | | |
| Early Response FOXJ1 high | FTO | FOXJ1 high Ciliated Cells | DLEC1 | SPRR2D high Squamous Cells | C15orf48 |
| Ciliated Cells | | | | | |
| Early Response FOXJ1 high | SLC25A4 | FOXJ1 high Ciliated Cells | EFCAB10 | SPRR2D high Squamous Cells | KRT17 |
| Ciliated Cells | | | | | |
| Early Response FOXJ1 high | CMTM6 | FOXJ1 high Ciliated Cells | TMBIM6 | SPRR2D high Squamous Cells | TMPRSS11E |
| Ciliated Cells | | | | | |
| Early Response FOXJ1 high | C21orf58 | FOXJ1 high Ciliated Cells | NEK5 | SPRR2D high Squamous Cells | KLK6 |
| Ciliated Cells | | | | | |
| Early Response FOXJ1 high | TUBGCP2 | FOXJ1 high Ciliated Cells | UNC119B | SPRR2D high Squamous Cells | TMPRSS11D |
| Ciliated Cells | | | | | |
| Early Response FOXJ1 high | CTNNAL1 | FOXJ1 high Ciliated Cells | NEK11 | SPRR2D high Squamous Cells | KRT16 |
| Ciliated Cells | | | | | |
| Early Response FOXJ1 high | PALLD | FOXJ1 high Ciliated Cells | AC013264.1 | SPRR2D high Squamous Cells | PRSS22 |
| Ciliated Cells | | | | | |
| Early Response FOXJ1 high | AGPAT3 | FOXJ1 high Ciliated Cells | ABCA13 | SPRR2D high Squamous Cells | LCN2 |
| Ciliated Cells | | | | | |
| Early Response FOXJ1 high | B9D2 | FOXJ1 high Ciliated Cells | ERGIC3 | SPRR2D high Squamous Cells | TMPRSS2 |
| Ciliated Cells | | | | | |
| Early Response FOXJ1 high | CFAP53 | FOXJ1 high Ciliated Cells | CALM1 | SPRR2D high Squamous Cells | EMP1 |
| Ciliated Cells | | | | | |
| Early Response FOXJ1 high | PCM1 | FOXJ1 high Ciliated Cells | ARL3 | SPRR2D high Squamous Cells | DUOX2 |
| Ciliated Cells | | | | | |
| Early Response FOXJ1 high | IRX3 | FOXJ1 high Ciliated Cells | CFAP221 | SPRR2D high Squamous Cells | SCEL |
| Ciliated Cells | | | | | |
| Early Response FOXJ1 high | GNA11 | FOXJ1 high Ciliated Cells | MAGED2 | SPRR2D high Squamous Cells | KLK10 |
| Ciliated Cells | | | | | |
| Early Response FOXJ1 high | SF3B2 | FOXJ1 high Ciliated Cells | ALDH1A1 | SPRR2D high Squamous Cells | MXD1 |
| Ciliated Cells | | | | | |
| Early Response FOXJ1 high | HSP90AA1 | FOXJ1 high Ciliated Cells | TBC1D8 | SPRR2D high Squamous Cells | SPNS2 |
| Ciliated Cells | | | | | |
| Early Response FOXJ1 high | CLU | FOXJ1 high Ciliated Cells | CABCOCO1 | SPRR2D high Squamous Cells | IL1RN |
| Ciliated Cells | | | | | |
| Early Response FOXJ1 high | GOLPH3L | FOXJ1 high Ciliated Cells | HDGF | SPRR2D high Squamous Cells | ERO1A |
| Ciliated Cells | | | | | |
| Early Response FOXJ1 high | C1orf158 | FOXJ1 high Ciliated Cells | CLMN | SPRR2D high Squamous Cells | ECM1 |
| Ciliated Cells | | | | | |
| Early Response FOXJ1 high | CYB561A3 | FOXJ1 high Ciliated Cells | PEBP1 | SPRR2D high Squamous Cells | CEACAM1 |
| Ciliated Cells | | | | | |
| Early Response FOXJ1 high | ACADM | FOXJ1 high Ciliated Cells | DYDC2 | SPRR2D high Squamous Cells | EPS8L1 |
| Ciliated Cells | | | | | |
| Early Response FOXJ1 high | NME5 | FOXJ1 high Ciliated Cells | MDH1B | SPRR2D high Squamous Cells | FAM129B |
| Ciliated Cells | | | | | |
| Early Response FOXJ1 high | BRD2 | FOXJ1 high Ciliated Cells | DNAH6 | SPRR2D high Squamous Cells | SPRR2F |
| Ciliated Cells | | | | | |
| Early Response FOXJ1 high | DMKN | FOXJ1 high Ciliated Cells | IFT27 | SPRR2D high Squamous Cells | PRSS27 |
| Ciliated Cells | | | | | |
| Early Response FOXJ1 high | DHCR24 | FOXJ1 high Ciliated Cells | CYB5D1 | SPRR2D high Squamous Cells | PDZK1IP1 |
| Ciliated Cells | | | | | |
| Early Response FOXJ1 high | PPP1R16A | FOXJ1 high Ciliated Cells | C11orf16 | SPRR2D high Squamous Cells | LMO7 |
| Ciliated Cells | | | | | |
| Early Response FOXJ1 high | HAGHL | FOXJ1 high Ciliated Cells | CTGF | SPRR2D high Squamous Cells | NCCRP1 |
| Ciliated Cells | | | | | |
| Early Response FOXJ1 high | FBXO15 | FOXJ1 high Ciliated Cells | CCDC81 | SPRR2D high Squamous Cells | PRSS8 |
| Ciliated Cells | | | | | |
| Early Response FOXJ1 high | TMEM9 | FOXJ1 high Ciliated Cells | TUSC3 | SPRR2D high Squamous Cells | TPM4 |
| Ciliated Cells | | | | | |
| Early Response FOXJ1 high | PPP1R14C | FOXJ1 high Ciliated Cells | STK33 | SPRR2D high Squamous Cells | ANXA1 |
| Ciliated Cells | | | | | |
| Early Response FOXJ1 high | TMEM245 | FOXJ1 high Ciliated Cells | AGBL2 | SPRR2D high Squamous Cells | SAT1 |
| Ciliated Cells | | | | | |
| Early Response FOXJ1 high | MANBAL | FOXJ1 high Ciliated Cells | C21orf58 | SPRR2D high Squamous Cells | SPRR1A |
| Ciliated Cells | | | | | |
| Early Response FOXJ1 high | CGN | FOXJ1 high Ciliated Cells | UCKL1-AS1 | SPRR2D high Squamous Cells | S100A12 |
| Ciliated Cells | | | | | |
| Early Response FOXJ1 high | ZNF487 | FOXJ1 high Ciliated Cells | MAATS1 | SPRR2D high Squamous Cells | KRT6C |
| Ciliated Cells | | | | | |
| Early Response FOXJ1 high | IFT140 | FOXJ1 high Ciliated Cells | CRIP2 | VEGFA high Squamous Cells | RPTN |
| Ciliated Cells | | | | | |
| Early Response FOXJ1 high | MORN3 | FOXJ1 high Ciliated Cells | NEK10 | VEGFA high Squamous Cells | UCA1 |
| Ciliated Cells | | | | | |
| Early Response FOXJ1 high | SORT1 | FOXJ1 high Ciliated Cells | ANKRD66 | VEGFA high Squamous Cells | CEACAM5 |
| Ciliated Cells | | | | | |
| Early Response FOXJ1 high | DUSP18 | FOXJ1 high Ciliated Cells | PPP1R16A | VEGFA high Squamous Cells | SPRR3 |
| Ciliated Cells | | | | | |
| Early Response FOXJ1 high | RFX2 | FOXJ1 high Ciliated Cells | IFT46 | VEGFA high Squamous Cells | MAL |
| Ciliated Cells | | | | | |
| Early Response FOXJ1 high | TMEM45B | FOXJ1 high Ciliated Cells | CFAP73 | VEGFA high Squamous Cells | HSPB8 |
| Ciliated Cells | | | | | |
| Early Response FOXJ1 high | TMEM232 | FOXJ1 high Ciliated Cells | RP1 | VEGFA high Squamous Cells | AKR1B1 |
| Ciliated Cells | | | | | |
| Early Response FOXJ1 high | PNMA1 | FOXJ1 high Ciliated Cells | NME5 | VEGFA high Squamous Cells | LAMB3 |
| Ciliated Cells | | | | | |
| Early Response FOXJ1 high | FAM120A | FOXJ1 high Ciliated Cells | TCTN1 | VEGFA high Squamous Cells | AZGP1 |
| Ciliated Cells | | | | | |
| Early Response FOXJ1 high | EMB | FOXJ1 high Ciliated Cells | SPTBN1 | VEGFA high Squamous Cells | MXD1 |
| Ciliated Cells | | | | | |
| Early Response FOXJ1 high | SERP1 | FOXJ1 high Ciliated Cells | HIPK1 | VEGFA high Squamous Cells | FAM129B |
| Ciliated Cells | | | | | |
| Early Response FOXJ1 high | DDX3Y | FOXJ1 high Ciliated Cells | CCDC96 | VEGFA high Squamous Cells | H1FO |
| Ciliated Cells | | | | | |
| Early Response FOXJ1 high | CCT2 | FOXJ1 high Ciliated Cells | CCDC74A | VEGFA high Squamous Cells | SCEL |
| Ciliated Cells | | | | | |
| Early Response FOXJ1 high | EPHX1 | FOXJ1 high Ciliated Cells | DNAH3 | VEGFA high Squamous Cells | FBXO32 |
| Ciliated Cells | | | | | |
| Early Response FOXJ1 high | TIMP4 | FOXJ1 high Ciliated Cells | SELENOW | VEGFA high Squamous Cells | SLC5A3 |
| Ciliated Cells | | | | | |
| Early Response FOXJ1 high | TAGLN2 | FOXJ1 high Ciliated Cells | CROCC | VEGFA high Squamous Cells | NCCRP1 |
| Ciliated Cells | | | | | |
| Early Response FOXJ1 high | MAP9 | FOXJ1 high Ciliated Cells | GPR162 | VEGFA high Squamous Cells | GNE |
| Ciliated Cells | | | | | |
| Early Response FOXJ1 high | HSP90AB1 | FOXJ1 high Ciliated Cells | SPAG17 | VEGFA high Squamous Cells | SPNS2 |
| Ciliated Cells | | | | | |
| Early Response FOXJ1 high | DNAJB4 | FOXJ1 high Ciliated Cells | CFAP46 | VEGFA high Squamous Cells | CDKN2B |
| Ciliated Cells | | | | | |
| Early Response FOXJ1 high | PARVA | FOXJ1 high Ciliated Cells | DNAJB2 | VEGFA high Squamous Cells | FTH1 |
| Ciliated Cells | | | | | |
| Early Response FOXJ1 high | CNPY3 | FOXJ1 high Ciliated Cells | TTC29 | VEGFA high Squamous Cells | EMP1 |
| Ciliated Cells | | | | | |
| Early Response FOXJ1 high | GLT8D1 | FOXJ1 high Ciliated Cells | C11orf97 | VEGFA high Squamous Cells | S100A4 |
| Ciliated Cells | | | | | |
| Early Response FOXJ1 high | BRD3OS | FOXJ1 high Ciliated Cells | ABHD2 | VEGFA high Squamous Cells | SPINT1 |
| Ciliated Cells | | | | | |
| Early Response FOXJ1 high | WDR13 | FOXJ1 high Ciliated Cells | CCDC40 | VEGFA high Squamous Cells | PPL |
| Ciliated Cells | | | | | |
| Early Response FOXJ1 high | TOGARAM1 | FOXJ1 high Ciliated Cells | CFAP70 | VEGFA high Squamous Cells | CEACAM6 |
| Ciliated Cells | | | | | |
| Early Response FOXJ1 high | GSTP1 | FOXJ1 high Ciliated Cells | VWA3A | VEGFA high Squamous Cells | TMPRSS11E |
| Ciliated Cells | | | | | |
| Early Response FOXJ1 high | FSD1L | FOXJ1 high Ciliated Cells | CD24 | VEGFA high Squamous Cells | TMPRSS11D |
| Ciliated Cells | | | | | |
| Early Response FOXJ1 high | DNAH9 | FOXJ1 high Ciliated Cells | RFX2 | VEGFA high Squamous Cells | CPA4 |
| Ciliated Cells | | | | | |
| Early Response FOXJ1 high | ZNF474 | FOXJ1 high Ciliated Cells | HYDIN | VEGFA high Squamous Cells | VEGFA |
| Ciliated Cells | | | | | |
| Early Response FOXJ1 high | JHY | FOXJ1 high Ciliated Cells | SAMD15 | VEGFA high Squamous Cells | KRT80 |
| Ciliated Cells | | | | | |
| Early Response FOXJ1 high | CYB5A | FOXJ1 high Ciliated Cells | CCDC180 | VEGFA high Squamous Cells | PITX1 |
| Ciliated Cells | | | | | |
| Early Response FOXJ1 high | CANX | FOXJ1 high Ciliated Cells | ERICH5 | VEGFA high Squamous Cells | OAS1 |
| Ciliated Cells | | | | | |
| Early Response FOXJ1 high | CFAP20 | FOXJ1 high Ciliated Cells | LRRC6 | VEGFA high Squamous Cells | CALB1 |
| Ciliated Cells | | | | | |
| Early Response FOXJ1 high | SLC25A36 | FOXJ1 high Ciliated Cells | CHST6 | VEGFA high Squamous Cells | MRPS6 |
| Ciliated Cells | | | | | |
| Early Response FOXJ1 high | ZNF295-AS1 | FOXJ1 high Ciliated Cells | DNAH11 | VEGFA high Squamous Cells | SAT1 |
| Ciliated Cells | | | | | |
| Early Response FOXJ1 high | ANKRD28 | FOXJ1 high Ciliated Cells | TOGARAM2 | VEGFA high Squamous Cells | TMPRSS2 |
| Ciliated Cells | | | | | |
| Early Response FOXJ1 high | ATP5F1A | FOXJ1 high Ciliated Cells | PROS1 | VEGFA high Squamous Cells | S100A9 |
| Ciliated Cells | | | | | |
| Early Response FOXJ1 high | CDC14A | FOXJ1 high Ciliated Cells | RCAN3 | VEGFA high Squamous Cells | MFSD4A |
| Ciliated Cells | | | | | |
| Early Response FOXJ1 high | LPAR3 | FOXJ1 high Ciliated Cells | PLEKHG7 | VEGFA high Squamous Cells | LMO7 |
| Ciliated Cells | | | | | |
| Early Response FOXJ1 high | PPP4R3B | FOXJ1 high Ciliated Cells | NFE2L1 | VEGFA high Squamous Cells | NDRG2 |
| Ciliated Cells | | | | | |
| Early Response FOXJ1 high | BAIAP2L1 | FOXJ1 high Ciliated Cells | NAT14 | VEGFA high Squamous Cells | ABCA1 |
| Ciliated Cells | | | | | |
| Early Response FOXJ1 high | TRIP13 | FOXJ1 high Ciliated Cells | SPAG8 | VEGFA high Squamous Cells | PRSS8 |
| Ciliated Cells | | | | | |
| Early Response FOXJ1 high | EBNA1BP2 | FOXJ1 high Ciliated Cells | WDR90 | VEGFA high Squamous Cells | TMBIM1 |
| Ciliated Cells | | | | | |
| Early Response FOXJ1 high | GLIS3 | FOXJ1 high Ciliated Cells | LRRIQ1 | VEGFA high Squamous Cells | DUSP5 |
| Ciliated Cells | | | | | |
| Early Response FOXJ1 high | ZC2HC1A | FOXJ1 high Ciliated Cells | FHAD1 | VEGFA high Squamous Cells | TP53INP2 |
| Ciliated Cells | | | | | |
| Early Response FOXJ1 high | BUD23 | FOXJ1 high Ciliated Cells | AZIN1 | VEGFA high Squamous Cells | SDCBP2 |
| Ciliated Cells | | | | | |
| Early Response FOXJ1 high | AHCYL1 | FOXJ1 high Ciliated Cells | PRKAR1A | VEGFA high Squamous Cells | ARHGAP5 |
| Ciliated Cells | | | | | |
| Early Response FOXJ1 high | DSTN | FOXJ1 high Ciliated Cells | PCM1 | VEGFA high Squamous Cells | ECM1 |
| Ciliated Cells | | | | | |
| Early Response FOXJ1 high | SMAP2 | FOXJ1 high Ciliated Cells | DAW1 | VEGFA high Squamous Cells | KRT23 |
| Ciliated Cells | | | | | |
| Early Response FOXJ1 high | COPS6 | FOXJ1 high Ciliated Cells | SHROOM3 | VEGFA high Squamous Cells | RIOK3 |
| Ciliated Cells | | | | | |
| Early Response FOXJ1 high | ODF2 | FOXJ1 high Ciliated Cells | DNAH1 | VEGFA high Squamous Cells | TRIP10 |
| Ciliated Cells | | | | | |
| Early Response FOXJ1 high | P4HA2 | FOXJ1 high Ciliated Cells | STOX1 | VEGFA high Squamous Cells | TIMP3 |
| Ciliated Cells | | | | | |
| Early Response FOXJ1 high | STMND1 | FOXJ1 high Ciliated Cells | CFAP36 | VEGFA high Squamous Cells | CST6 |
| Ciliated Cells | | | | | |
| Early Response FOXJ1 high | CCT7 | FOXJ1 high Ciliated Cells | KIF19 | VEGFA high Squamous Cells | RALA |
| Ciliated Cells | | | | | |
| Early Response FOXJ1 high | C16orf71 | FOXJ1 high Ciliated Cells | ANKMY1 | VEGFA high Squamous Cells | PRDM1 |
| Ciliated Cells | | | | | |
| Early Response FOXJ1 high | NDUFA8 | FOXJ1 high Ciliated Cells | ARHGAP18 | VEGFA high Squamous Cells | CLTB |
| Ciliated Cells | | | | | |
| Early Response FOXJ1 high | GON7 | FOXJ1 high Ciliated Cells | AHSA1 | VEGFA high Squamous Cells | SLK |
| Ciliated Cells | | | | | |
| Early Response FOXJ1 high | CRIP2 | FOXJ1 high Ciliated Cells | GLB1L | VEGFA high Squamous Cells | SQSTM1 |
| Ciliated Cells | | | | | |
| Early Response FOXJ1 high | MCAT | FOXJ1 high Ciliated Cells | IL5RA | VEGFA high Squamous Cells | APP |
| Ciliated Cells | | | | | |
| Early Response FOXJ1 high | SLC6A6 | FOXJ1 high Ciliated Cells | DMKN | VEGFA high Squamous Cells | MTUS1 |
| Ciliated Cells | | | | | |
| Early Response FOXJ1 high | PLCH1 | FOXJ1 high Ciliated Cells | CFAP126 | VEGFA high Squamous Cells | ITGB8 |
| Ciliated Cells | | | | | |
| Early Response FOXJ1 high | HIBADH | FOXJ1 high Ciliated Cells | LRRC74B | VEGFA high Squamous Cells | PRSS27 |
| Ciliated Cells | | | | | |
| Early Response FOXJ1 high | HSPBP1 | FOXJ1 high Ciliated Cells | TSNAXIP1 | VEGFA high Squamous Cells | PRSS22 |
| Ciliated Cells | | | | | |
| Early Response FOXJ1 high | ANXA7 | FOXJ1 high Ciliated Cells | MLF1 | VEGFA high Squamous Cells | LYPD3 |
| Ciliated Cells | | | | | |
| Early Response FOXJ1 high | BCAS3 | FOXJ1 high Ciliated Cells | ENPP4 | VEGFA high Squamous Cells | FOSL2 |
| Ciliated Cells | | | | | |
| Early Response FOXJ1 high | AKAP6 | FOXJ1 high Ciliated Cells | HACD4 | VEGFA high Squamous Cells | EPHA2 |
| Ciliated Cells | | | | | |
| Early Response FOXJ1 high | RHOA | FOXJ1 high Ciliated Cells | SPACA9 | VEGFA high Squamous Cells | ANXA11 |
| Ciliated Cells | | | | | |
| Early Response FOXJ1 high | SLC22A23 | FOXJ1 high Ciliated Cells | IQCE | VEGFA high Squamous Cells | PHACTR2 |
| Ciliated Cells | | | | | |
| Early Response FOXJ1 high | KIF19 | FOXJ1 high Ciliated Cells | C1orf87 | VEGFA high Squamous Cells | PLS3 |
| Ciliated Cells | | | | | |
| Early Response FOXJ1 high | CAP2 | FOXJ1 high Ciliated Cells | C12orf75 | VEGFA high Squamous Cells | NECTIN4 |
| Ciliated Cells | | | | | |
| Early Response FOXJ1 high | GCLM | FOXJ1 high Ciliated Cells | WLS | VEGFA high Squamous Cells | FHL2 |
| Ciliated Cells | | | | | |
| Early Response FOXJ1 high | ISCA2 | FOXJ1 high Ciliated Cells | MNS1 | VEGFA high Squamous Cells | ZNF185 |
| Ciliated Cells | | | | | |
| Early Response FOXJ1 high | ELK3 | FOXJ1 high Ciliated Cells | VPS35 | VEGFA high Squamous Cells | TMEM106B |
| Ciliated Cells | | | | | |
| Early Response FOXJ1 high | NEK10 | FOXJ1 high Ciliated Cells | LZTFL1 | VEGFA high Squamous Cells | IL1RN |
| Ciliated Cells | | | | | |
| Early Response FOXJ1 high | VDAC3 | FOXJ1 high Ciliated Cells | WDR63 | VEGFA high Squamous Cells | C6orf132 |
| Ciliated Cells | | | | | |
| Early Response FOXJ1 high | EIF5 | FOXJ1 high Ciliated Cells | DPCD | VEGFA high Squamous Cells | CAMK2N1 |
| Ciliated Cells | | | | | |
| Early Response FOXJ1 high | PRR18 | FOXJ1 high Ciliated Cells | CCDC69 | VEGFA high Squamous Cells | YPEL5 |
| Ciliated Cells | | | | | |
| Early Response FOXJ1 high | LPGAT1 | FOXJ1 high Ciliated Cells | DNAL1 | VEGFA high Squamous Cells | CAP1 |
| Ciliated Cells | | | | | |
| Early Response FOXJ1 high | LCA5 | FOXJ1 high Ciliated Cells | MAP9 | VEGFA high Squamous Cells | RANBP9 |
| Ciliated Cells | | | | | |
| Early Response FOXJ1 high | TPPP | FOXJ1 high Ciliated Cells | TSPAN19 | VEGFA high Squamous Cells | TMPRSS11A |
| Ciliated Cells | | | | | |
| Early Response FOXJ1 high | WDR35 | FOXJ1 high Ciliated Cells | LRTOMT | VEGFA high Squamous Cells | KLK10 |
| Ciliated Cells | | | | | |
| Early Response FOXJ1 high | KIAA1191 | FOXJ1 high Ciliated Cells | TNFRSF19 | VEGFA high Squamous Cells | GALNT5 |
| Ciliated Cells | | | | | |
| Early Response FOXJ1 high | NORAD | FOXJ1 high Ciliated Cells | ATXN7L3B | VEGFA high Squamous Cells | CPEB4 |
| Ciliated Cells | | | | | |
| Early Response FOXJ1 high | IFT43 | FOXJ1 high Ciliated Cells | RIPOR2 | VEGFA high Squamous Cells | CCNG2 |
| Ciliated Cells | | | | | |
| Early Response FOXJ1 high | HSPH1 | FOXJ1 high Ciliated Cells | DNAH7 | VEGFA high Squamous Cells | PCDH1 |
| Ciliated Cells | | | | | |
| Early Response FOXJ1 high | RGS22 | FOXJ1 high Ciliated Cells | KIAA2012 | VEGFA high Squamous Cells | LGALS3 |
| Ciliated Cells | | | | | |
| Early Response FOXJ1 high | ARSD | FOXJ1 high Ciliated Cells | BAIAP2L1 | VEGFA high Squamous Cells | DIAPH1 |
| Ciliated Cells | | | | | |
| Early Response FOXJ1 high | DPY30 | FOXJ1 high Ciliated Cells | CLUAP1 | VEGFA high Squamous Cells | ST3GAL4 |
| Ciliated Cells | | | | | |
| Early Response FOXJ1 high | PDLIM4 | FOXJ1 high Ciliated Cells | MYCBP | VEGFA high Squamous Cells | HECA |
| Ciliated Cells | | | | | |
| Early Response FOXJ1 high | IFT27 | FOXJ1 high Ciliated Cells | CFAP74 | VEGFA high Squamous Cells | SLCO4A1 |
| Ciliated Cells | | | | | |
| Early Response FOXJ1 high | EML1 | FOXJ1 high Ciliated Cells | CCDC78 | VEGFA high Squamous Cells | B3GALT5 |
| Ciliated Cells | | | | | |
| Early Response FOXJ1 high | CAB39 | FOXJ1 high Ciliated Cells | TCTEX1D1 | VEGFA high Squamous Cells | SPECC1 |
| Ciliated Cells | | | | | |
| Early Response FOXJ1 high | PALMD | FOXJ1 high Ciliated Cells | TEKT2 | VEGFA high Squamous Cells | LAMA4 |
| Ciliated Cells | | | | | |
| Early Response FOXJ1 high | SEC14L1 | FOXJ1 high Ciliated Cells | RAB36 | VEGFA high Squamous Cells | JUP |
| Ciliated Cells | | | | | |
| Early Response FOXJ1 high | TMC5 | FOXJ1 high Ciliated Cells | PTPRF | VEGFA high Squamous Cells | TIMP2 |
| Ciliated Cells | | | | | |
| Early Response FOXJ1 high | DDAH1 | FOXJ1 high Ciliated Cells | CAT | VEGFA high Squamous Cells | TACC1 |
| Ciliated Cells | | | | | |
| Early Response FOXJ1 high | TMEM14B | FOXJ1 high Ciliated Cells | COQ4 | VEGFA high Squamous Cells | MAFF |
| Ciliated Cells | | | | | |
| Early Response FOXJ1 high | KDM1B | FOXJ1 high Ciliated Cells | ERBB4 | VEGFA high Squamous Cells | UBE2R2 |
| Ciliated Cells | | | | | |
| Early Response FOXJ1 high | HDLBP | FOXJ1 high Ciliated Cells | STEAP3 | VEGFA high Squamous Cells | HOPX |
| Ciliated Cells | | | | | |
| Early Response FOXJ1 high | IARS2 | FOXJ1 high Ciliated Cells | LRRC71 | VEGFA high Squamous Cells | NPC1 |
| Ciliated Cells | | | | | |
| Early Response FOXJ1 high | 9-Sep | FOXJ1 high Ciliated Cells | DCDC2B | VEGFA high Squamous Cells | ABHD5 |
| Ciliated Cells | | | | | |
| Early Response FOXJ1 high | RALB | FOXJ1 high Ciliated Cells | COPRS | VEGFA high Squamous Cells | CYSRT1 |
| Ciliated Cells | | | | | |
| Early Response FOXJ1 high | CFAP36 | FOXJ1 high Ciliated Cells | SPATA17 | VEGFA high Squamous Cells | VWF |
| Ciliated Cells | | | | | |
| Early Response FOXJ1 high | DCBLD2 | FOXJ1 high Ciliated Cells | ORAI2 | VEGFA high Squamous Cells | LAD1 |
| Ciliated Cells | | | | | |
| Early Response FOXJ1 high | DCDC2B | FOXJ1 high Ciliated Cells | CMTM4 | VEGFA high Squamous Cells | STRN |
| Ciliated Cells | | | | | |
| Early Response FOXJ1 high | PFKP | FOXJ1 high Ciliated Cells | RAB3B | VEGFA high Squamous Cells | OXSR1 |
| Ciliated Cells | | | | | |
| Early Response FOXJ1 high | ARHGAP39 | FOXJ1 high Ciliated Cells | PDLIM1 | VEGFA high Squamous Cells | METRNL |
| Ciliated Cells | | | | | |
| Early Response FOXJ1 high | C17orf97 | FOXJ1 high Ciliated Cells | C20orf96 | VEGFA high Squamous Cells | CNNM1 |
| Ciliated Cells | | | | | |
| Early Response FOXJ1 high | FAM149A | FOXJ1 high Ciliated Cells | TMEM232 | VEGFA high Squamous Cells | TINCR |
| Ciliated Cells | | | | | |
| Early Response FOXJ1 high | C1orf87 | FOXJ1 high Ciliated Cells | GAPVD1 | VEGFA high Squamous Cells | ITPRIP |
| Ciliated Cells | | | | | |
| Early Response FOXJ1 high | OAZ1 | FOXJ1 high Ciliated Cells | CROCC2 | VEGFA high Squamous Cells | SPHK1 |
| Ciliated Cells | | | | | |
| Early Response FOXJ1 high | CFLAR | FOXJ1 high Ciliated Cells | SF3B2 | VEGFA high Squamous Cells | PLEKHF1 |
| Ciliated Cells | | | | | |
| Early Response FOXJ1 high | PRUNE2 | FOXJ1 high Ciliated Cells | AKAP14 | VEGFA high Squamous Cells | CRYBG2 |
| Ciliated Cells | | | | | |
| Early Response FOXJ1 high | GMPR2 | FOXJ1 high Ciliated Cells | DRC7 | VEGFA high Squamous Cells | GRB7 |
| Ciliated Cells | | | | | |
| Early Response FOXJ1 high | SCRN1 | FOXJ1 high Ciliated Cells | DNAH12 | VEGFA high Squamous Cells | VSIG10L |
| Ciliated Cells | | | | | |
| Early Response FOXJ1 high | FILIP1 | | | | |
| Ciliated Cells | | | | | |

Table 1C. Detailed Immune Cell types (see FIG. 9)

| | | | | | |
|---|---|---|---|---|---|
| B Cells | MS4A1 | Early Response T Cells | SARAF | Inflammatory Macrophages | CXCL8 |
| B Cells | IGHM | Early Response T Cells | DUSP16 | Inflammatory Macrophages | IL1B |
| B Cells | BANK1 | Early Response T Cells | MAP1A | Inflammatory Macrophages | CCL3L1 |
| B Cells | FCRL5 | Early Response T Cells | SLC7A5 | Inflammatory Macrophages | CCL3 |
| B Cells | CD79A | Early Response T Cells | SNHG7 | Inflammatory Macrophages | CXCL2 |
| B Cells | IGKC | Early Response T Cells | ATF4 | Inflammatory Macrophages | PLIN2 |
| B Cells | MEF2C | Early Response T Cells | PIP4K2A | Inflammatory Macrophages | CCL20 |
| B Cells | CD22 | Early Response T Cells | UBB | Inflammatory Macrophages | CXCL3 |
| B Cells | TNFRSF13B | Early Response T Cells | BCOR | Inflammatory Macrophages | SOD2 |
| B Cells | CD19 | Early Response T Cells | ELF1 | Inflammatory Macrophages | TNFAIP6 |
| B Cells | CXCR5 | Early Response T Cells | TOB2 | Inflammatory Macrophages | IER3 |
| B Cells | PAX5 | Early Response T Cells | DYNLL1 | Inflammatory Macrophages | PLEK |
| B Cells | CD74 | Early Response T Cells | DYNLL2 | Inflammatory Macrophages | SNX10 |
| CD8 T Cells | SYNE2 | Early Response T Cells | HSP90AB1 | Inflammatory Macrophages | BAG3 |
| CD8 T Cells | XIST | Early Response T Cells | NCL | Inflammatory Macrophages | G0S2 |
| CD8 T Cells | RORA | Early Response T Cells | SNHG12 | Inflammatory Macrophages | CCL4 |
| CD8 T Cells | EVL | Early Response T Cells | LDLRAD4 | Inflammatory Macrophages | SGK1 |
| CD8 T Cells | CD2 | Early Response T Cells | TIPARP | Inflammatory Macrophages | AC243829.4 |
| CD8 T Cells | MT-ND6 | Early Response T Cells | PDCD4 | Inflammatory Macrophages | ZFP36 |
| CD8 T Cells | ETS1 | Early Response T Cells | HSPA8 | Inflammatory Macrophages | AQP9 |
| CD8 T Cells | CD96 | Early Response T Cells | CITED2 | Inflammatory Macrophages | KLF10 |
| CD8 T Cells | MTRNR2L1 | Early Response T Cells | CNOT6L | Inflammatory Macrophages | NFKBIA |
| CD8 T Cells | MT-ND5 | Early Response T Cells | ZC3HAV1 | Inflammatory Macrophages | IL1RN |
| CD8 T Cells | TC2N | Early Response T Cells | CDKN1B | Inflammatory Macrophages | BCL2A1 |
| CD8 T Cells | SYNE1 | Early Response T Cells | CCDC88C | Inflammatory Macrophages | CCRL2 |
| CD8 T Cells | KMT2A | Early Response T Cells | CALM1 | Inflammatory Macrophages | FTL |
| CD8 T Cells | MT-CYB | Early Response T Cells | ITK | Inflammatory Macrophages | FTH1 |
| CD8 T Cells | PARP8 | Early Response T Cells | PITHD1 | Inflammatory Macrophages | HMOX1 |
| CD8 T Cells | MT-ND2 | Early Response T Cells | ICOS | Inflammatory Macrophages | EREG |
| CD8 T Cells | RBL2 | Early Response T Cells | FBXO32 | Inflammatory Macrophages | CEBPB |
| CD8 T Cells | MT-ND1 | Early Response T Cells | SLC38A1 | Inflammatory Macrophages | CCL4L2 |
| CD8 T Cells | MT-ND4L | Early Response T Cells | LCK | Inflammatory Macrophages | PLAUR |
| CD8 T Cells | ARAP2 | Early Response T Cells | ERRFI1 | Inflammatory Macrophages | WTAP |
| CD8 T Cells | MT-ATP6 | Early Response T Cells | UBALD2 | Inflammatory Macrophages | ANXA5 |
| CD8 T Cells | CD69 | Early Response T Cells | IVNS1ABP | Inflammatory Macrophages | NAMPT |
| CD8 T Cells | MACF1 | Early Response T Cells | TRAC | Inflammatory Macrophages | NBN |
| CD8 T Cells | BTN3A1 | Early Response T Cells | TRIM26 | Inflammatory Macrophages | ZC3H12C |
| CD8 T Cells | CLEC2D | Early Response T Cells | ZNF683 | Inflammatory Macrophages | ICAM1 |
| CD8 T Cells | ACAP1 | Early Response T Cells | RYBP | Inflammatory Macrophages | BEST1 |
| CD8 T Cells | ANKRD44 | Early Response T Cells | SCML4 | Inflammatory Macrophages | C15orf48 |
| CD8 T Cells | STK17B | Early Response T Cells | CDR2 | Inflammatory Macrophages | CD68 |
| CD8 T Cells | MT-ND4 | Early Response T Cells | MYADM | Inflammatory Macrophages | CXCL1 |
| CD8 T Cells | IKZF1 | Early Response T Cells | SPOCK2 | Inflammatory Macrophages | MARCKS |
| CD8 T Cells | PDCD4 | Early Response T Cells | G3BP2 | Inflammatory Macrophages | CCL2 |
| CD8 T Cells | MGAT4A | Early Response T Cells | SIRT1 | Inflammatory Macrophages | CD83 |
| CD8 T Cells | CCL5 | Early Response T Cells | RASGRP1 | Inflammatory Macrophages | HSPA6 |
| CD8 T Cells | MT-CO1 | Early Response T Cells | SPTY2D1 | Inflammatory Macrophages | OLR1 |
| CD8 T Cells | SMCHD1 | Early Response T Cells | HMGCS1 | Inflammatory Macrophages | SDCBP |
| CD8 T Cells | AHNAK | Early Response T Cells | LDHA | Inflammatory Macrophages | NFKBIZ |
| CD8 T Cells | KIAA1551 | Early Response T Cells | PREX1 | Inflammatory Macrophages | RASGEF1B |
| CD8 T Cells | MT-ATP8 | Early Response T Cells | CAMK4 | Inflammatory Macrophages | TNFAIP2 |
| CD8 T Cells | AKAP9 | Early Response T Cells | CD96 | Inflammatory Macrophages | ATP2B1 |
| CD8 T Cells | UTRN | Early Response T Cells | JMY | Inflammatory Macrophages | BHLHE40 |
| CD8 T Cells | RNF213 | Early Response T Cells | HIPK1 | Inflammatory Macrophages | HSPA1A |
| CD8 T Cells | PTPN7 | Early Response T Cells | SKI | Inflammatory Macrophages | MXD1 |
| CD8 T Cells | SMARCA2 | Early Response T Cells | PTP4A1 | Inflammatory Macrophages | NINJ1 |
| CD8 T Cells | SPTAN1 | Early Response T Cells | DDX24 | Inflammatory Macrophages | NFE2L2 |
| CD8 T Cells | EMB | Early Response T Cells | SRSF2 | Inflammatory Macrophages | ETS2 |
| CD8 T Cells | TAGAP | Early Response T Cells | KDM3A | Inflammatory Macrophages | SQSTM1 |
| CD8 T Cells | EPB41 | Early Response T Cells | GNAS | Inflammatory Macrophages | DUSP1 |
| CD8 T Cells | HELZ | Early Response T Cells | SNRK | Inflammatory Macrophages | RIPK2 |
| CD8 T Cells | HIPK1 | Early Response T Cells | STMN1 | Inflammatory Macrophages | KYNU |
| CD8 T Cells | MT-ND3 | Early Response T Cells | CD3G | Inflammatory Macrophages | ZNF267 |
| CD8 T Cells | MT-CO3 | Early Response T Cells | USP38 | Inflammatory Macrophages | SPP1 |
| CD8 T Cells | NCOA2 | Early Response T Cells | EIF4A3 | Inflammatory Macrophages | HSPE1 |
| CD8 T Cells | DYNLL2 | Early Response T Cells | DUSP5 | Inflammatory Macrophages | SLC39A8 |
| CD8 T Cells | PTPRC | Early Response T Cells | ATP2B4 | Inflammatory Macrophages | C5AR1 |
| CD8 T Cells | BPTF | Early Response T Cells | CDV3 | Inflammatory Macrophages | MMP19 |
| CD8 T Cells | TNRC6B | Early Response T Cells | JADE1 | Inflammatory Macrophages | TIMP1 |
| CD8 T Cells | SYNRG | Early Response T Cells | KDM2A | Inflammatory Macrophages | CTSZ |
| CD8 T Cells | MT-CO2 | Early Response T Cells | TMX4 | Inflammatory Macrophages | CD63 |
| CD8 T Cells | MBNL1 | Early Response T Cells | ZFAS1 | Inflammatory Macrophages | LHFPL2 |
| CD8 T Cells | FKBP5 | Early Response T Cells | AHSA1 | Inflammatory Macrophages | CTSL |
| CD8 T Cells | DDX6 | Early Response T Cells | GADD45A | Inflammatory Macrophages | MAPK6 |
| CD8 T Cells | CDC42SE2 | Early Response T Cells | PNRC1 | Inflammatory Macrophages | ABL2 |
| CD8 T Cells | PCM1 | Early Response T Cells | TUBA1B | Inflammatory Macrophages | PIM3 |
| CD8 T Cells | SARAF | Early Response T Cells | PBXIP1 | Inflammatory Macrophages | LGALS3 |
| CD8 T Cells | GOLGB1 | Early Response T Cells | LNPEP | Inflammatory Macrophages | PHACTR1 |
| CD8 T Cells | DEF6 | Early Response T Cells | AKNA | Inflammatory Macrophages | PSAP |
| CD8 T Cells | ITGA4 | Early Response T Cells | IFFO2 | Inflammatory Macrophages | CLEC5A |
| CD8 T Cells | OGA | Early Response T Cells | PAG1 | Inflammatory Macrophages | HSPA1B |
| CD8 T Cells | CNTRL | Early Response T Cells | MPZL3 | Inflammatory Macrophages | CD44 |
| CD8 T Cells | PRPF38B | Early Response T Cells | NDRG1 | Inflammatory Macrophages | PPP1R15A |
| CD8 T Cells | EIF4A2 | Early Response T Cells | SF3A1 | Inflammatory Macrophages | IFNGR2 |
| CD8 T Cells | DDX24 | Early Response T Cells | GTF2B | Inflammatory Macrophages | ATP6V1B2 |
| CD8 T Cells | MYCBP2 | Early Response T Cells | IL2RB | Inflammatory Macrophages | GSTO1 |
| CD8 T Cells | PPP2R5C | Early Response T Cells | KPNA2 | Inflammatory Macrophages | DNAJB1 |
| CD8 T Cells | SETD2 | Early Response T Cells | PPP1R16B | Inflammatory Macrophages | LIMS1 |
| CD8 T Cells | STK10 | Early Response T Cells | SRSF3 | Inflammatory Macrophages | LYZ |
| CD8 T Cells | DOCK8 | Early Response T Cells | RNF125 | Inflammatory Macrophages | CTSB |
| CD8 T Cells | IDS | Early Response T Cells | SPRY1 | Inflammatory Macrophages | CLEC4E |
| CD8 T Cells | FNBP1 | Early Response T Cells | BCL11B | Inflammatory Macrophages | CSTB |
| CD8 T Cells | HECA | Early Response T Cells | PRKCH | Inflammatory Macrophages | CD300E |
| CD8 T Cells | EPC1 | Early Response T Cells | SLC2A1 | Inflammatory Macrophages | MAFF |
| CD8 T Cells | KMT2C | Early Response T Cells | RANBP2 | Inflammatory Macrophages | VIM |
| CD8 T Cells | SRSF7 | Early Response T Cells | RHOH | Inflammatory Macrophages | S100A8 |
| CD8 T Cells | SRSF11 | Early Response T Cells | POLRZA | Inflammatory Macrophages | NPC2 |
| CD8 T Cells | CALM1 | Early Response T Cells | PLEKHA2 | Inflammatory Macrophages | FNIP2 |
| CD8 T Cells | NIN | Early Response T Cells | DDX3X | Inflammatory Macrophages | AC015912.3 |
| CD8 T Cells | G3BP2 | Early Response T Cells | NFATC2 | Inflammatory Macrophages | FCER1G |
| CD8 T Cells | CEP350 | Early Response T Cells | FOXO1 | Inflammatory Macrophages | ASAH1 |
| CD8 T Cells | TERF2IP | Early Response T Cells | HSPA9 | Inflammatory Macrophages | MAFB |
| CD8 T Cells | DDX3X | Early Response T Cells | DNAJA1 | Inflammatory Macrophages | CYBB |
| CD8 T Cells | GOLGA4 | Early Response T Cells | RBL2 | Inflammatory Macrophages | ATP6V1F |
| CD8 T Cells | SKI | Early Response T Cells | SERINC1 | Inflammatory Macrophages | S100A9 |
| CD8 T Cells | NCOR1 | Early Response T Cells | SEMA4D | Inflammatory Macrophages | LYN |
| CD8 T Cells | RBM25 | Early Response T Cells | GNG2 | Inflammatory Macrophages | SERPINA1 |
| CD8 T Cells | IL6ST | Early Response T Cells | ARIH1 | Inflammatory Macrophages | APLP2 |
| CD8 T Cells | ANKRD12 | Early Response T Cells | ATP1A1 | Inflammatory Macrophages | LGALS1 |
| CD8 T Cells | MATR3 | Early Response T Cells | CCNT1 | Inflammatory Macrophages | CTSS |
| CD8 T Cells | DDX17 | Early Response T Cells | IKZF1 | Inflammatory Macrophages | FCGR2A |
| CD8 T Cells | ELOVL5 | Early Response T Cells | ANKRD28 | Inflammatory Macrophages | ACTB |
| CD8 T Cells | CHD2 | Early Response T Cells | GATA3 | Inflammatory Macrophages | ANXA2 |
| CD8 T Cells | VPS13C | Early Response T Cells | SRSF7 | Inflammatory Macrophages | SERF2 |
| CD8 T Cells | MIER1 | Early Response T Cells | IKZF3 | Inflammatory Macrophages | TYROBP |
| CD8 T Cells | SETX | Early Response T Cells | IDI1 | Inflammatory Macrophages | SAT1 |
| CD8 T Cells | GSTK1 | Early Response T Cells | ETS1 | Inflammatory Macrophages | EMILIN2 |
| CD8 T Cells | KDM2A | Early Response T Cells | CLEC2D | Inflammatory Macrophages | S100A11 |
| CD8 T Cells | TAF7 | Early Response T Cells | SAMSN1 | Inflammatory Macrophages | ATP5F1E |
| CD8 T Cells | PPP1R2 | Early Response T Cells | TERF2IP | Inflammatory Macrophages | DMXL2 |
| CD8 T Cells | CNBP | Early Response T Cells | RPS3 | Inflammatory Macrophages | FCN1 |
| CD8 T Cells | WNK1 | Early Response T Cells | TBX21 | Inflammatory Macrophages | AIF1 |
| CD8 T Cells | FXR1 | Early Response T Cells | SLC30A1 | Inflammatory Macrophages | VCAN |
| CD8 T Cells | PPP4R3B | Early Response T Cells | PIM2 | Inflammatory Macrophages | ANPEP |
| CD8 T Cells | NOP53 | Early Response T Cells | UBC | Inflammatory Macrophages | CD14 |
| CD8 T Cells | CELF2 | Early Response T Cells | FYN | Inflammatory Macrophages | ABCA1 |
| CD8 T Cells | IVNS1ABP | Early Response T Cells | SLC38A2 | Inflammatory Macrophages | CTSD |
| CD8 T Cells | GLS | Early Response T Cells | C12orf65 | Inflammatory Macrophages | CD163 |
| CD8 T Cells | KDM5A | Early Response T Cells | CD3E | Inflammatory Macrophages | SLC11A1 |
| CD8 T Cells | DYNC1H1 | Early Response T Cells | THEMIS | Inflammatory Macrophages | HLA-DRA |
| CD8 T Cells | HNRNPH1 | Early Response T Cells | NOP56 | Interferon Responsive Cytotoxic | GNLY |
| | | | | CD8 T Cells | |
| CD8 T Cells | SUPT5H | Early Response T Cells | TC2N | Interferon Responsive Cytotoxic | GZMB |
| | | | | CD8 T Cells | |
| CD8 T Cells | SLC38A2 | Early Response T Cells | CD2 | Interferon Responsive Cytotoxic | GZMA |
| | | | | CD8 T Cells | |
| CD8 T Cells | NUP98 | Early Response T Cells | GSPT1 | Interferon Responsive Cytotoxic | PRF1 |
| | | | | CD8 T Cells | |
| CD8 T Cells | ARID4B | Early Response T Cells | CEP85L | Interferon Responsive Cytotoxic | LAG3 |
| | | | | CD8 T Cells | |
| CD8 T Cells | MYH9 | Early Response T Cells | HSPA5 | Interferon Responsive Cytotoxic | IL2RB |
| | | | | CD8 T Cells | |
| CD8 T Cells | PCBP2 | Early Response T Cells | MBNL1 | Interferon Responsive Cytotoxic | NKG7 |
| | | | | CD8 T Cells | |
| CD8 T Cells | SMARCA5 | Early Response T Cells | CREM | Interferon Responsive Cytotoxic | ISG20 |
| | | | | CD8 T Cells | |
| CD8 T Cells | SERINC1 | Early Response T Cells | PPM1K | Interferon Responsive Cytotoxic | APOBEC3G |
| | | | | CD8 T Cells | |
| CD8 T Cells | AHR | Early Response T Cells | PRKCQ | Interferon Responsive Cytotoxic | CD247 |
| | | | | CD8 T Cells | |
| CD8 T Cells | CCDC47 | Early Response T Cells | SUN2 | Interferon Responsive Cytotoxic | GBP4 |
| | | | | CD8 T Cells | |
| CD8 T Cells | RBMX | Early Response T Cells | AKIRIN1 | Interferon Responsive Cytotoxic | CD7 |
| | | | | CD8 T Cells | |
| CD8 T Cells | RPL5 | Early Response T Cells | SAP18 | Interferon Responsive Cytotoxic | IFITM1 |
| | | | | CD8 T Cells | |
| CD8 T Cells | SF3B1 | Early Response T Cells | WNK1 | Interferon Responsive Cytotoxic | TRBC2 |
| | | | | CD8 T Cells | |
| CD8 T Cells | KMT2E | Early Response T Cells | SMARCA2 | Interferon Responsive Cytotoxic | HSH2D |
| | | | | CD8 T Cells | |
| CD8 T Cells | PRRC2C | Early Response T Cells | PARP8 | Interferon Responsive | CXCL10 |
| | | | | Macrophages | |
| CD8 T Cells | CLK1 | Early Response T Cells | TRGC2 | Interferon Responsive | ISG15 |
| | | | | Macrophages | |
| CD8 T Cells | CREM | Early Response T Cells | GOLGB1 | Interferon Responsive | GBP1 |
| | | | | Macrophages | |
| CD8 T Cells | SF1 | Early Response T Cells | TOB1 | ITGAX high Macrophages | VCAN |
| CD8 T Cells | RPS4X | Early Response T Cells | SPTAN1 | ITGAX high Macrophages | S100A9 |
| CD8 T Cells | HNRNPA0 | Early Response T Cells | ITGAE | ITGAX high Macrophages | SLC11A1 |
| CD8 T Cells | ELF1 | Early Response T Cells | RCAN3 | ITGAX high Macrophages | S100A8 |
| CD8 T Cells | ZMYM5 | Early Response T Cells | VCP | ITGAX high Macrophages | TIMP1 |
| CD8 T Cells | SET | Early Response T Cells | TUBA1A | ITGAX high Macrophages | ITGAX |
| CD8 T Cells | DDX5 | Early Response T Cells | HERC1 | ITGAX high Macrophages | RETN |
| CD8 T Cells | PPP1R12A | Early Response T Cells | CHD2 | ITGAX high Macrophages | FCER1G |
| CD8 T Cells | CHD4 | Early Response T Cells | KLRC1 | ITGAX high Macrophages | ANPEP |
| CD8 T Cells | CCT2 | Early Response T Cells | CD247 | ITGAX high Macrophages | HES1 |
| CD8 T Cells | SERINC3 | Early Response T Cells | ATP1B1 | ITGAX high Macrophages | SERPINA1 |
| CD8 T Cells | DNM2 | Early Response T Cells | PIK3CD | ITGAX high Macrophages | CCL2 |
| CD8 T Cells | C16orf72 | Early Response T Cells | TFRC | ITGAX high Macrophages | FCN1 |
| CD8 T Cells | RAD21 | Early Response T Cells | PRKACB | ITGAX high Macrophages | NEAT1 |
| CD8 T Cells | SLK | Early Response T Cells | RNF10 | ITGAX high Macrophages | MAFB |
| CD8 T Cells | UBXN4 | Early Response T Cells | EPB41 | ITGAX high Macrophages | CTSL |
| CD8 T Cells | PRNP | Early Response T Cells | MGAT5 | ITGAX high Macrophages | BEST1 |
| CD8 T Cells | RPL3 | Early Response T Cells | EIF4G2 | ITGAX high Macrophages | SLC43A2 |
| CD8 T Cells | SRSF2 | Early Response T Cells | RPL18 | ITGAX high Macrophages | CD14 |
| CD8 T Cells | AKIRIN1 | Early Response T Cells | NOP53 | ITGAX high Macrophages | CTSB |
| CD8 T Cells | HP1BP3 | Early Response T Cells | TBC1D10C | ITGAX high Macrophages | DMXL2 |
| CD8 T Cells | SMG1 | Early Response T Cells | SKAP1 | ITGAX high Macrophages | STAB1 |
| CD8 T Cells | NAP1L1 | Early Response T Cells | GBP5 | ITGAX high Macrophages | CSTB |
| CD8 T Cells | GSPT1 | Early Response T Cells | FAM102A | ITGAX high Macrophages | CD163 |
| CD8 T Cells | AKAP13 | Early Response T Cells | TSEN54 | ITGAX high Macrophages | TYROBP |
| CD8 T Cells | RTRAF | Early Response T Cells | RANBP9 | ITGAX high Macrophages | AIF1 |
| CD8 T Cells | RNF10 | Early Response T Cells | CDKN2AIP | ITGAX high Macrophages | ZEB2 |
| CD8 T Cells | HLA-F | Early Response T Cells | CDC42EP3 | ITGAX high Macrophages | LILRB2 |
| CD8 T Cells | ANXA1 | Early Response T Cells | CELF2 | ITGAX high Macrophages | CTSS |
| CD8 T Cells | PTBP1 | Early Response T Cells | TGFBR2 | ITGAX high Macrophages | SLC25A37 |
| CD8 T Cells | ARIH1 | Early Response T Cells | ARHGEF3 | ITGAX high Macrophages | S100A11 |
| CD8 T Cells | ZNF207 | Early Response T Cells | STK10 | ITGAX high Macrophages | LILRB1 |
| CD8 T Cells | JAK1 | Early Response T Cells | RPS12 | ITGAX high Macrophages | LRP1 |
| CD8 T Cells | TAF1D | Early Response T Cells | ARID5A | ITGAX high Macrophages | CD63 |
| CD8 T Cells | RSRP1 | Early Response T Cells | IDS | ITGAX high Macrophages | TYMP |
| CD8 T Cells | PPP1CB | Early Response T Cells | PPP2R5C | ITGAX high Macrophages | ABCA1 |
| CD8 T Cells | NONO | Early Response T Cells | OXNAD1 | ITGAX high Macrophages | LYZ |
| CD8 T Cells | TAX1BP1 | Early Response T Cells | RPS4X | ITGAX high Macrophages | TGFBI |
| CD8 T Cells | SKP1 | Early Response T Cells | EEF2 | ITGAX high Macrophages | FTH1 |
| CD8 T Cells | SERTAD2 | Early Response T Cells | ACAP1 | ITGAX high Macrophages | CLEC5A |
| Dendritic Cells | CCR7 | Early Response T Cells | JAML | ITGAX high Macrophages | SLC39A8 |
| Dendritic Cells | HLA-DPB1 | Early Response T Cells | ARHGAP9 | ITGAX high Macrophages | SH3BGRL3 |
| Dendritic Cells | HLA-DQA1 | Early Response T Cells | FAM214A | ITGAX high Macrophages | FPR1 |
| Dendritic Cells | HLA-DRA | Early Response T Cells | ZFAND5 | ITGAX high Macrophages | FTL |
| Dendritic Cells | HLA-DPA1 | Early Response T Cells | CNBP | ITGAX high Macrophages | LGALS1 |
| Dendritic Cells | HLA-DRB1 | Early Response T Cells | SBDS | ITGAX high Macrophages | TLR2 |
| Dendritic Cells | HLA-DQB1 | Early Response T Cells | CD6 | ITGAX high Macrophages | AC020656.1 |
| Dendritic Cells | HLA-DQA2 | Early Response T Cells | NSD3 | ITGAX high Macrophages | CTSD |
| Dendritic Cells | CD74 | Early Response T Cells | EIF5 | ITGAX high Macrophages | CYBA |
| Dendritic Cells | HLA-DRB5 | Early Response T Cells | PPP1R2 | ITGAX high Macrophages | CCDC88A |
| Dendritic Cells | GPR183 | Early Response T Cells | 9-Sep | ITGAX high Macrophages | S100A12 |
| Dendritic Cells | CD83 | Early Response T Cells | USP25 | ITGAX high Macrophages | FNDC3B |
| Dendritic Cells | HLA-DQB2 | Early Response T Cells | EIF4A2 | ITGAX high Macrophages | C15orf48 |
| Dendritic Cells | LAMP3 | Early Response T Cells | IP6K2 | ITGAX high Macrophages | FGR |
| Dendritic Cells | CD86 | Early Response T Cells | KMT2A | ITGAX high Macrophages | CD68 |
| Dendritic Cells | GPR157 | Early Response T Cells | 1-Sep | ITGAX high Macrophages | PLAUR |
| Dendritic Cells | DAPP1 | Early Response T Cells | PSD4 | ITGAX high Macrophages | PSAP |
| Dendritic Cells | NR4A3 | Early Response T Cells | PDE7A | ITGAX high Macrophages | EMP3 |
| Dendritic Cells | CSF2RA | Early Response T Cells | CDC42SE2 | ITGAX high Macrophages | NCF2 |
| Dendritic Cells | PPA1 | Early Response T Cells | PTPN4 | ITGAX high Macrophages | HCK |
| Dendritic Cells | KLF4 | Early Response T Cells | SYTL3 | ITGAX high Macrophages | GSTO1 |
| Dendritic Cells | RALA | Early Response T Cells | PTPRC | ITGAX high Macrophages | MCEMP1 |
| Dendritic Cells | CDKN1A | Early Response T Cells | HLA-B | ITGAX high Macrophages | HM13 |
| Dendritic Cells | ETV3 | Early Response T Cells | RPL3 | ITGAX high Macrophages | DDX60L |
| Dendritic Cells | BASP1 | Early Response T Cells | KMT2E | ITGAX high Macrophages | EMILIN2 |
| Dendritic Cells | VEGFA | Early Response T Cells | AMD1 | ITGAX high Macrophages | TNS3 |
| Dendritic Cells | CST3 | Early Response T Cells | RAD21 | ITGAX high Macrophages | CLEC4E |
| Dendritic Cells | FAM49A | Early Response T Cells | SMURF2 | ITGAX high Macrophages | LUCAT1 |
| Dendritic Cells | TMSB4X | Early Response T Cells | LASP1 | ITGAX high Macrophages | DAB2 |
| Dendritic Cells | REL | Early Response T Cells | NACA | ITGAX high Macrophages | CD300E |
| Dendritic Cells | LITAF | Early Response T Cells | RPS16 | ITGAX high Macrophages | SERF2 |
| Dendritic Cells | SERPINB9 | Early Response T Cells | ARAP2 | ITGAX high Macrophages | NUMB |
| Dendritic Cells | TTYH2 | Early Response T Cells | C16orf72 | ITGAX high Macrophages | PDXK |
| Dendritic Cells | CLIC2 | Early Response T Cells | ATP8A1 | ITGAX high Macrophages | LILRB3 |
| Dendritic Cells | CTSZ | Early Response T Cells | ATP5F1B | ITGAX high Macrophages | PFN1 |
| Dendritic Cells | PLEK | Early Response T Cells | DNAJB9 | ITGAX high Macrophages | IGSF6 |
| Early Response T Cells | NR4A2 | Early Response T Cells | PCNX1 | ITGAX high Macrophages | TNFRSF1B |
| Early Response T Cells | TNFAIP3 | Early Response T Cells | BIRC3 | ITGAX high Macrophages | APLP2 |
| Early Response T Cells | FOSB | Early Response T Cells | ZAP70 | ITGAX high Macrophages | IFITM3 |
| Early Response T Cells | TSC22D3 | Early Response T Cells | DUSP1 | ITGAX high Macrophages | SPI1 |
| Early Response T Cells | CCL5 | Early Response T Cells | RPL4 | ITGAX high Macrophages | ATP13A3 |
| Early Response T Cells | RGCC | Early Response T Cells | SELENOK | ITGAX high Macrophages | P2RX4 |
| Early Response T Cells | ZNF331 | Early Response T Cells | UHRF2 | ITGAX high Macrophages | NPC2 |
| Early Response T Cells | CSF1 | Early Response T Cells | EEF1D | ITGAX high Macrophages | CD93 |
| Early Response T Cells | FAM53C | Early Response T Cells | TBL1XR1 | ITGAX high Macrophages | ASAH1 |
| Early Response T Cells | ZFP36L2 | Early Response T Cells | CALM2 | ITGAX high Macrophages | S100A6 |
| Early Response T Cells | BTG1 | Early Response T Cells | AZIN1 | ITGAX high Macrophages | SH3BP2 |
| Early Response T Cells | ODC1 | Early Response T Cells | YWHAZ | ITGAX high Macrophages | LAIR1 |
| Early Response T Cells | CSRNP1 | Early Response T Cells | CBLB | ITGAX high Macrophages | OLR1 |
| Early Response T Cells | SOD1 | Early Response T Cells | KIAA1551 | ITGAX high Macrophages | ANXA5 |
| Early Response T Cells | NR4A3 | Early Response T Cells | BUD31 | ITGAX high Macrophages | AQP9 |
| Early Response T Cells | NEU1 | Early Response T Cells | ITGAL | ITGAX high Macrophages | CTSA |
| Early Response T Cells | ITGA1 | Early Response T Cells | H3F3B | ITGAX high Macrophages | ZMIZ1 |
| Early Response T Cells | YPEL5 | Early Response T Cells | NLRC5 | ITGAX high Macrophages | LILRB4 |
| Early Response T Cells | EZR | Early Response T Cells | CCSER2 | ITGAX high Macrophages | SIRPA |
| Early Response T Cells | BRD2 | Early Response T Cells | CLEC2B | ITGAX high Macrophages | GRN |
| Early Response T Cells | DEDD2 | Early Response T Cells | SF1 | ITGAX high Macrophages | GABARAP |
| Early Response T Cells | DUSP4 | Early Response T Cells | PIK3IP1 | ITGAX high Macrophages | ATP5F1E |
| Early Response T Cells | NR4A1 | Early Response T Cells | PRF1 | ITGAX high Macrophages | LYN |
| Early Response T Cells | CD69 | Early Response T Cells | HERPUD2 | ITGAX high Macrophages | PTPRE |
| Early Response T Cells | BTG2 | Early Response T Cells | FNBP1 | ITGAX high Macrophages | TSPO |
| Early Response T Cells | TUBA4A | Early Response T Cells | PPP1R15A | ITGAX high Macrophages | LGALS3 |
| Early Response T Cells | STK17B | Early Response T Cells | SLC20A1 | ITGAX high Macrophages | TCIRG1 |
| Early Response T Cells | CXCR4 | Early Response T Cells | EVL | ITGAX high Macrophages | MARCKS |
| Early Response T Cells | KLF6 | Early Response T Cells | PTPN7 | ITGAX high Macrophages | LILRA6 |
| Early Response T Cells | TENT5C | Early Response T Cells | RPS5 | ITGAX high Macrophages | MYL6 |
| Early Response T Cells | TUBB4B | Early Response T Cells | RASGEF1B | ITGAX high Macrophages | S100A10 |
| Early Response T Cells | ATF3 | Early Response T Cells | ANKRD44 | ITGAX high Macrophages | BRI3 |
| Early Response T Cells | SESN2 | Early Response T Cells | FYB1 | ITGAX high Macrophages | VMP1 |
| Early Response T Cells | PER1 | Early Response T Cells | RPLP0 | ITGAX high Macrophages | IRAK1 |
| Early Response T Cells | TAGAP | Early Response T Cells | SMARCA5 | ITGAX high Macrophages | EFHD2 |
| Early Response T Cells | MYLIP | Early Response T Cells | SYNRG | ITGAX high Macrophages | DSE |
| Early Response T Cells | WDR47 | Early Response T Cells | CIRBP | ITGAX high Macrophages | ATP6V0B |
| Early Response T Cells | CCND2 | Early Response T Cells | KDM5A | ITGAX high Macrophages | LIMS1 |
| Early Response T Cells | CD8A | Early Response T Cells | C6orf48 | ITGAX high Macrophages | PCF11 |
| Early Response T Cells | TSPYL2 | Early Response T Cells | RPL8 | ITGAX high Macrophages | MALAT1 |
| Early Response T Cells | IFNG | Early Response T Cells | WDR26 | ITGAX high Macrophages | TNIP1 |
| Early Response T Cells | NEDD9 | Early Response T Cells | HNRNPDL | ITGAX high Macrophages | CST3 |
| Early Response T Cells | IRF4 | Early Response T Cells | TAF1D | ITGAX high Macrophages | PKM |
| Early Response T Cells | SERTAD1 | Early Response T Cells | MATR3 | ITGAX high Macrophages | GPX4 |
| Early Response T Cells | FOSL2 | Early Response T Cells | CORO1A | ITGAX high Macrophages | ZYX |
| Early Response T Cells | LBH | Early Response T Cells | PNN | ITGAX high Macrophages | H2AFY |
| Early Response T Cells | CBX4 | Early Response T Cells | CNTRL | ITGAX high Macrophages | S100A4 |
| Early Response T Cells | MCL1 | Early Response T Cells | NOP58 | ITGAX high Macrophages | C6orf48 |
| Early Response T Cells | MKNK2 | Early Response T Cells | PRRC2C | ITGAX high Macrophages | TPM4 |
| Early Response T Cells | RGS1 | Early Response T Cells | SYNE1 | ITGAX high Macrophages | SAT1 |
| Early Response T Cells | TRBC2 | FFAR4 high Macrophages | MTRNR2L8 | ITGAX high Macrophages | ACTB |
| Early Response T Cells | PTPN22 | FFAR4 high Macrophages | MRC1 | ITGAX high Macrophages | SRGN |
| Early Response T Cells | ZFP36L1 | FFAR4 high Macrophages | MTRNR2L6 | ITGAX high Macrophages | VIM |
| Early Response T Cells | DUSP2 | FFAR4 high Macrophages | MTRNR2L3 | ITGAX high Macrophages | CFL1 |
| Early Response T Cells | BCL2L11 | FFAR4 high Macrophages | MTRNR2L10 | ITGAX high Macrophages | FLNA |
| Early Response T Cells | STK17A | FFAR4 high Macrophages | APOE | ITGAX high Macrophages | GRB2 |
| Early Response T Cells | SLC2A3 | FFAR4 high Macrophages | FABP4 | ITGAX high Macrophages | SRRM2 |
| Early Response T Cells | PNP | FFAR4 high Macrophages | ALDH2 | ITGAX high Macrophages | SOD2 |
| Early Response T Cells | JUN | FFAR4 high Macrophages | FN1 | ITGAX high Macrophages | TPM3 |
| Early Response T Cells | SYTL2 | FFAR4 high Macrophages | CFD | ITGAX high Macrophages | CCL3 |
| Early Response T Cells | HECA | FFAR4 high Macrophages | CHIT1 | MSR1 C1QB high Macrophages | APOC1 |
| Early Response T Cells | CD3D | FFAR4 high Macrophages | CCL18 | MSR1 C1QB high Macrophages | C1QA |
| Early Response T Cells | SMAD7 | FFAR4 high Macrophages | CYP27A1 | MSR1 C1QB high Macrophages | C1QB |
| Early Response T Cells | NLRC3 | FFAR4 high Macrophages | CAMP | MSR1 C1QB high Macrophages | GPNMB |
| Early Response T Cells | PDE4D | FFAR4 high Macrophages | FHL1 | MSR1 C1QB high Macrophages | LGMN |
| Early Response T Cells | TRBC1 | FFAR4 high Macrophages | BEX3 | MSR1 C1QB high Macrophages | MSR1 |
| Early Response T Cells | GABARAPL1 | FFAR4 high Macrophages | PROS1 | MSR1 C1QB high Macrophages | PSAP |
| Early Response T Cells | PRNP | FFAR4 high Macrophages | SIGLEC11 | MSR1 C1QB high Macrophages | C1QC |
| Early Response T Cells | JUND | FFAR4 high Macrophages | CHI3L1 | MSR1 C1QB high Macrophages | A2M |
| Early Response T Cells | EHD1 | FFAR4 high Macrophages | SLAMF9 | MSR1 C1QB high Macrophages | CTSB |
| Early Response T Cells | RORA | FFAR4 high Macrophages | FFAR4 | MSR1 C1QB high Macrophages | TREM2 |
| Early Response T Cells | PTGER4 | FFAR4 high Macrophages | AC126365.1 | MSR1 C1QB high Macrophages | TIMP2 |
| Early Response T Cells | NASP | FFAR4 high Macrophages | RPS4Y2 | MSR1 C1QB high Macrophages | SLCO2B1 |
| Early Response T Cells | SLC9A3R1 | FFAR4 high Macrophages | PARAL1 | MSR1 C1QB high Macrophages | GAA |
| Early Response T Cells | KIAA1191 | FFAR4 high Macrophages | SLC47A1 | | |

TABLE 2
Differentially Expressed Genes Between Cell Types from Control WHO 0 vs. COVID-19 WHO 1-5 (Mild/Moderate).
Related to FIG. 3. Results from the comparison of cells from each cell type between Control WHO 0 vs. COVID-19
WHO 1-5 (mild/moderate) individuals. (Implemented using the FindAllMarkers function in Seurat, test.use =
“negbinom”; Genes included with adjusted pvalue < 0.001, logFC > 0.25; Cell types without
sufficient cells to test or fewer than 5 significant genes meeting the cutoffs are not listed).

Table 2A. Expressed in COVID-19 WHO 1-5 (mild/moderate) individuals
Basal Cells
HSPA5, HERPUD1, MAFF, CLDN4, GADD45A, ZFP36, JUND, MCL1, IFI27, MYLIP, EGR1, CCNL1, UBC, SERTAD1, EMP1,
TXNRD1, KLF6, IFI6, TACSTD2, CDKN1A, GADD45B, RAB11FIP1, HLA-E, SLC25A25, HSPH1, KRT6A, VMO1, INSIG1,
DDIT3
Ciliated Cells (all)
IFI6, IFI27, IFITM3, ISG15, IFI44L, MX1, IFIT1, XAF1, SLPI, HLA-C, PARP14, HLA-A, IFITM1, IFI44, OAS2, IFIT3, STAT1,
HLA-E, LAP3, LY6E, HERC6, PARP9, MX2, OAS3, SCO2, OAS1, TRIM22, PLSCR1, CD74, HLA-F, IFITM2, LGALS3BP, BST2,
WFDC2, CMPK2, SPATS2L, SAMD9, HLA-B, RSAD2, UBE2L6, B2M, TMSB10, S100A6, S100A11, GBP1, ODF3B, TUBB4B,
EIF2AK2, DDX60, MT2A, ISG20, OMG, TUBA1A, UNC93B1, EPSTI1, APOL1, SLFN5, CTSH, TAP1, IFI16, CFAP126,
SMIM22, TNFSF10, SCGB2A1, ADAR, PSMB8, METTL7A, EIF5A, IFIT2, RARRES3, HLA-DRA, APOL6, TSPAN1, PSME2,
COX5A, SAMD9L, COX6A1, FXYD3, FTL, TYMP, MUC1, JTB, HRASLS2, SHISA5, PSMB9, HSPH1, RNF213, DYNLL1, KRT19,
STAT2, NUPR1, C1orf194, ROMO1, SERPING1, MDK, AD000090.1, CTSS, PSCA, POLR2I, WARS, AL357093.2, LAMP3,
SP100, STOM, S100P, GAPDH, DDX58, AES, COX5B, TAPBP, FAM183A, ATP5F1E, HIST1H1C, HINT1, MPV17L, NDUFA4,
PSMB3, NUCB2, SAT1, COX7A2, CXCL17, ATP1B1, PIGR, SLC6A6, IK, HDGF, CTSD, PTMA, PLAC8, ANXA11, ATP5MG,
RHBDD2, COX6B1, SP110, DDX24, CHCHD10, COX7C, NDUFB1, MUC15, PSENEN, GSTK1, FAU, DHCR24, TRIM69,
NFE2L2, NDUFB4, EIF1, NFKBIA, GSN, DTX3L, KRT8, COX8A, ATP5ME, TUBA1B, MORN2, C9orf116, COX6C, ATP5MC2,
OXTR, ACTG1, NAA38, IGFBP2, UQCR11, HSP90B1, ZNFX1, HLA-DRB1, HLA-DPA1, TPI1, PSME1, DHRS9, MDH2,
TRIM8, SLC2A1, UBB, SERF2, COX7B, AGR3, FLNB, PPP1CB, SKP1, PPDPF, RPN2, ITM2B, TPPP3, TMEM14B, PEBP1,
TSPAN3, CAST, UQCRQ, IRF1, ATP6AP1, ATP5MPL, NDUFB9, YWHAE, ATP5IF1, TFF3, CDCP1, OPTN, BCL2L1, ENSA,
PGD, SSR4, UQCR10, CALR, CD9, FDFT1, NDUFA2, TMEM219, CFAP36, ATP5F1B, LRRC10B, SRP14, GBP3, CAPS,
PALLD, TALDO1, IFIH1, PDIA3, CD82, TMEM231, LAPTM4A, QSOX1, C1orf43, LDLR, FAM229B, CD99, TOMM7,
DYNLT1, SNTN, ELOB, SSB, F11R, PRSS23, NUDC, SELENOH, POR, PLXNB2, PLEKHS1, NUB1, CNDP2, SLC35A2,
TMEM14C, ACTB, HSPA5, BRI3, ACO2, IFT22, CSTB, DUOX2, CPEB4, FAM81B, S100A10, ANXA1, CUTA, CFL1, CETN2,
CDC42, ATP6V0E1, CYP2S1, ARPC3, CFAP298, SPINT2, TMED10, NACA, CBR1, SRSF3, SNU13, STOML3, KIF9, TSTD1,
TACSTD2, TMED4, FAM92B, C3, DHX32, SMAP2, IDH2, DBI, PTGES3, TUSC3, ZFAS1, SPCS1, ANXA2, CD81, RPN1,
ERGIC3, TSPAN13, SNRNP25, WRB, TMEM50A, TMED9, TRIM29, ENO1, BUD23, ZMYND10, UBL5, RER1, CTGF, IFT57,
OSCP1, TRAF4, SEC14L1, NDUFS3, ATP2A2, HNRNPA2B1, MGST3, TXN, MDM2, UBA52, WDR1, STUB1, PPIB, CHCHD2,
SAP18, AC007906.2, ACTN4, DYNLRB1, TMEM59, LYN, NDUFA1, ZMAT2, MYL12B, TMBIM4, ALCAM, ATP6V1G1,
PSMA4, HSP90AB1, TMEM173, H3F3B, NDUFAB1, SF3B2, SAMHD1, TUFM, MTF1, TMEM258, C4orf3, TAX1BP1, CES1,
GPX4, VNN3, CYSTM1, KIAA1522, RAB36, DNTTIP2, GADD45GIP1, ATP6V0B, EDF1, NDUFB10, SIX1, ATP5PD, BTF3,
PKM, UBC, IRX3, HSPD1, HSPBP1, SARAF, RILPL2, NDUFS5, 9-Sep, RIBC2, PRDX6, SQLE, RAD23A, ELF3, BRD2, FIS1,
HDLBP, GNB2, VAPA, IDS, CST3, SQSTM1, RAB11FIP1, CLCN3, ATP5MD, IFT46, PFDN5, SPA17, CAPSL, UCP2, CHMP5,
CMPK1, SLC7A2, SLC25A5, EPHX1, ATF4, RTN3, BUD31, CALM2, SLC9A3R1, GUK1, PRDX1, SLC25A6, HNRNPM, RTN4,
ATP5F1A, XRCC5, ATP1A1, COX4I1, FTH1, SMDT1, CHMP2A, KLF6, GSR, WDR45B, XRCC6, PTP4A2, ESRRA, GTF2F1,
DAD1, FUS, PSMD2, CTNNAL1, TXNIP, CIRBP, NME5, C20orf85, CYB561, NUCKS1, SELENOW, C9orf24, UQCRC1,
TMED2, ARL3, P4HB, PERP, YPEL5, DYNLL2, CAPNS1, PFN2, LGALS3, DDR1, CYB561A3, UPF1, MRFAP1, MAL2, EIF4G1,
MLEC, TJP3, SSBP1, FAM107B, DNAJA1, CDS1, CD24, RABGAP1L, PABPC1, CIR1, ROPN1L, DNAJB1, MUC20, MCL1,
BASP1, C9orf135, OCIAD1, VMO1, CES2, CCT6A, KTN1, CD63, PDIA4, NQO1, WDR54, MEA1, CABCOCO1, SCAF11,
NAT14, ASRGL1, HNRNPK, PIGT, GOLPH3L, TNFAIP8L1, GNG5, GSTP1, TMEM123, HNRNPD, MVP, COQ4, TNFRSF21,
NDRG2, EIF3D, CAPN1, REEP5, NDUFB2, RSPH1, SSU72, CFAP300, NFE2L1, ARF1, ERLEC1, HSP90AA1, B3GNT7, LARP1,
PDLIM1, BCAP31, CFLAR, CIB1, GNB1, HMGN3, PSAP, TEKT1, SPTBN1, SET, AHSA1, P4HTM, PPM1G, ISCU, ST14,
NEDD8, DEGS2, UFC1, C5orf49, ANKRD54, GLB1L, BPIFA1, BAIAP2, HSPA9, VAMP8, HSBP1, TMBIM6, PKIG,
HNRNPUL1, ERH, HACD3, AQP3, SLC31A1, LMAN2, RAC1, CCDC113, CCDC80, PTTG1IP, JPT2, DNPH1, TGOLN2,
DNALI1, SDC4, C6orf132, CLDN7, EEF2, PPP1R15A, CSNK1A1, DDB1, CASC4, ABLIM1, CDH1, YWHAB, XBP1, CTSB,
GOLM1, RUVBL1, RAB11A, SYAP1, EPCAM, APP, SLC25A3, STAT3, SPATA18, TSC22D1, CCT2, KIF5B, LRTOMT,
B4GALT1, MSI2, ARPC2, ALDH3A1, ADH7, BBX, PSMB5, CCDC65, CD38, CLIC1, KIAA0319L, PCBP1, TM9SF2, ZFAND5,
MYL12A, MTDH, PLPP2, ADIPOR1, EZR, PCBP2, ARL6IP4, ERP29, LRP10, GADD45B, SLC20A2, VDAC3, PLTP, SLAIN2,
TMX4, FAM96B, CLINT1, ALKBH5, IQCG, NARS, UBXN4, YWHAZ, TMC4, CHMP4B, SOD1, TPT1, GSTA1, MID1IP1,
PTBP3, DIAPH2, CCNI, BAG6, PFKP, BBOF1, WDR90, MORN5, DLG3, AGPAT3, HLA-DRB5, CLSTN1, CANX, GABARAPL2,
EIF4G2, CCPG1, THRAP3, EEF1D, DDX3Y, RBM47, C11orf97, SLC44A4, RND3, EBNA1BP2, DSTN, YBX3, CERS6, UBE2H,
CALM1, STAU1, TRIM2, TMF1, CD164, PDIA6, KPNB1, CTNNA1, OS9, PIFO, VPS35, ITGA2, SMIM14, GDI2, KHDRBS1,
DPY30, DPCD, UBE2D3, PPP4R3B, PPL, MAP1A, PAPOLA, RACK1, WDR34, COPB2, ALDH1A1, LMO7, KRT18, CLTA,
TPM3, MLF1, SSRP1, MGLL, CMTM6, HSPA1B, OAZ1, TUBA4B, MAP3K19, PPP1R7, LRP11, VCP, CSDE1, CCT3, DNAJB2,
TTC25, LRRC23, PTPRF, RHOA, ZNF664, EXPH5, SPTLC2, PARK7, MPC2, MYL6, ALDH3B1, ILF3, PSMB4, TAGLN2, KIF3B,
AKR1C3, SERPINB1, RSPH9, GHITM, RAB7A, CDKN1A, MORF4L2, RRAD, ANXA7, RBM3, PROM1, KIF2A, GAPVD1, NCL,
EIF5, GNAS, ICMT, MATR3, FAM120A, MYH14, IQGAP1, RUVBL2, GLUL, SLC27A2, KLF5, B3GNT5, ERICH3, AKR1A1,
CCDC170, SRI, JUN, FOXJ1, LRRFIP1, EIF3A, C2orf40, UBXN10, CFAP45, ENAH, LDLRAD1, CCDC69, PPIL6, C12orf75,
GNS, LDHB, RSPH4A, EGR1, CLDN3, CKB, PCM1, MFSD6, DAZAP2, ZNF106, ATP12A, ATF3, CC2D2A, TM9SF3, CHST6,
NWD1, PRKAR1A, SAA1, AKR1C2, ABHD2, CYP4B1, APLP2, CHD4, EHF, HSPA8, SELENBP1, ALOX15, CHST9, SPAG6,
AGR2, AP2B1, KIF21A, TBC1D8, ENKUR, C11orf88, MAP6, TMEM190, HIPK1, GSTA2, RHOB, CLTC, CD59, SAA2,
TXNRD1, PRDX5, TSPAN6, LCN2, FMO3, HSPA1A, FOS
Developing Ciliated Cells
HSP90AA1, IFI6, HSP90AB1, HIST1H1C, HLA-B, HSP90B1, CD74, AGR2, IFI27, HSPH1, SERF2, B2M, HLA-DRA, ENKUR,
STAT1, KTN1, HSPA8, HLA-C, LAP3, HLA-E, TXN, SAMHD1, CFAP53, CCDC146, HSPA4, CALM1, CANX, IQCG, DZIP3,
HSPD1, LDLRAD1, PCM1, KIF21A, CAST, PSCA, C20orf85, S100A4, S100A10, CCDC113, ABLIM1, SYNE2, PARP14,
TMA7, TMEM212, HLA-A, IK, DYNLL1, NUDC, FMO3, DNAJA4, SCGB2A1, MMACHC, HSPA5, UGT2A1, CFAP36, IFITM3,
ATP1B1, PTGES3, HLA-DRB1, DNAJA1, GRAMD2B, MUC16, TSPAN1, UPF1, NWD1, HSPE1, CCT3, C4orf3, GBP1,
TUBA1A, ANXA2, TYMP, ALCAM, STIP1, CETN2, C11orf88, HNRNPU, ARMC3, MT2A, SERPINB3, YBX1, UFC1, STAU1,
PPIL6, RIBC2, ZNF106, YWHAE, HSBP1, WARS, SKP1, HNRNPA2B1, ATP5F1E, SOD1, SAMD15, CBR1, CLIP1, CYP4B1,
COX6A1, SPTLC2, SPATS2L, ARHGAP18, SNX3, ATP5MG, CALR, HNRNPC, FTL, CFAP45, MLEC, ABCA13, RSPH1,
CC2D2A, AHNAK, DYNLRB2, ARL3, SNTN, THRAP3, GOLGB1, ATP2A2, DDX17, BEST1, ADGRF1, JPT2, CFAP298, MNS1,
ST13, COX7A2, PTMA, PDCD6IP, PPP4R3B, MX1, PDIA3, MATR3, EIF4A2, TPR, SEM1, TP53BP1, AKR1A1, COX7C,
GOLM1, NME5, SLFN5, HSPA9, PABPC1, FAM216B, UQCRQ, DHRS9, TPT1, CFAP43, TMSB4X, SON, SARAF, CAPSL,
CDHR3, MAP1A, KRT19, TM9SF2, ALDH1A1, LCN2, HECTD1, TSPAN3, C1orf194, AC007906.2, SSB, FAM166A,
SECISBP2L, HDLBP, PIGR, GCC2, RSPH3, LRP11, MORF4L2, BBOF1, SPATA18, ANXA1, SMIM22, PARK7, PTBP3, PIFO,
WDR78, ACTG1, SRP14, CLTC, CHL1, RSPH4A, CDC42, ABHD2, TUBB4B, SPEF2, SLPI, GON7, GNAS, TPPP3, CTNNA1,
YWHAZ, PKM, EIF3A, FTH1, RBM39, S100A11, APLP2, DSTN, NPHP1, ENAH, CCDC65, HNRNPK, AKAP9, KIF3B, TRIP12,
DPY30, EEF1A1, KRT8, GAPDH, UGDH, LDHB, DBI, PRRC2C, TMEM59, ADH7, TSPAN6, TM9SF3, UQCR11, HNRNPF,
VWA3B, KIF5B, ZMYND10, CD59, VPS35, CDH1, C9orf116, CFAP300, CD24, SAXO2, CDS1, AZIN1, TMED10, NFE2L1,
CEP126, DYNLT1, HIPK1, CCDC170, SPA17, MDM2, TAGLN2, TNFAIP8L1, UBE2H, NDUFA4, CLINT1, DNALI1, LMO7,
UBL5, TMF1, SPTBN1, MSI2, HMGN3, WDR66, AL357093.2, YWHAB, LRRFIP1, TAX1BP1, AGR3, FHAD1, LRRIQ1,
EFCAB1, VAPA, PSENEN, MORF4L1, MYL6, NQO1, ATP1A1, C9orf24, BASP1, MYL12B, NUCB2, EIF4G2, RFX3, OMG,
CD9, ACTB, SYAP1, TMEM123, MAP3K19, MORN2, AK7, IFT57, CIB1, SARSCoV2-3prime, GSTP1, DNAH12, IGFBP5,
PPP1CB, FAM183A, ATP5IF1, CFAP44, PSAP, PERP, TMBIM6, SPAG6, H3F3B, CCDC80, UBB, EZR
Goblet Cells (all)
IFI27, IFI6, MX1, XAF1, IFI44L, IFI44, PARP14, ISG15, STAT1, HERC6, IFITM1, IFITM3, EPSTI1, IFIT1,
PARP9, BST2, IFIT3, EIF2AK2, MX2, OAS3, OAS2, LGALS3BP, TRIM22, DDX60, HLA-C, HLA-E, LY6E, SAMHD1,
VMO1, CYP2A13, EPAS1, SLC31A1, SP100, APOL6, PRSS23, HLA-A, AQP5, MSMB, PSCA, ADAR, F3, TMEM213,
TNFSF10, GSN, UGT2A1, CD82
Ionocytes
IFI27, CD74, HLA-C, IFI6, TMSB10, HLA-E, STAT1, PRDX1, DBI, HLA-F, CTSS, ENAM, TXNRD1, S100A6
Macrophages (all)
CXCL2, CXCL3, B2M, HLA-C, HLA-B, STAT1, HLA-DRA, GBP1, HLA-DPA1, HLA-A, CD74
Secretory Cells (all)
IFI27, MX1, IFI6, IFITM3, XAF1, PARP14, STAT1, ISG15, TNFSF10, HLA-E, VMO1, IFITM1, HLA-C, HERC6, IFI44L, MSMB,
IFI44, CD74, WARS, RNF213, FTL, IFIT3, SP100, MDK, PARP9, HLA-DRA, IFIT1, BST2, FAM107B, BPIFB1, EIF2AK2, OAS2,
SAMHD1, DUOX2, HLA-A, EPSTI1, APOL1, LAP3, HLA-DRB5, HSP90AA1, ADAM28, ADAR, HLA-B, FBP1, ID2, GOLGA4,
MTUS1, S100A4, ATF4, FMO3, STAT2, RARRES3, DNAJA1, GOLGB1, LY6E, KTN1
Squamous Cells (all)
IFI6, IFI27, ISG15, CD74, IFIT3, RSAD2, IFIT1, XAF1,
WARS

Table 2B. Expressed in Control WHO 0 individuals
Basal Cells
COL7A1, KRT15
Ciliated Cells (all)
FRMPD2
Developing Ciliated Cells
GSTA2
Goblet Cells (all)
ANKRD36C, LINC00342, DST, ABCA13, STATH
Squamous Cells (all)
EEF1A1, ATP1B3, ABCA13, MALAT1, MACF1, AHNAK, SUN1, SF3B1, S100A7,
CNN3, SERPINB4, DNAH5, SERPINB3, SYNE1, KLK13, FTH1

TABLE 3
Differentially Expressed Genes Between Cell Types from Control WHO 0 vs. COVID-19 WHO 6-8 (Severe). Related to FIG. 3. Results
from the comparison of cells from each cell type between Control WHO 0 vs. COVID-19 WHO 6-8 (severe) individuals. (Implemented
using the FindAllMarkers function in Seurat, test.use = “negbinom”; Genes included with adjusted pvalue <
0.001, logFC > 0.25; Cell types without sufficient cells to test or fewer than 5 significant genes meeting the cutoffs are not listed).

Table 3A. Expressed in COVID-19 WHO 6-8 (severe) individuals
Basal Cells
HLA-C, ADH1C, NEBL, IFI27, VMO1, FMO2, HLA-B, HLA-A, XAF1, CHL1, CYP4B1, HLA-E, KIF21A, EGR1, B2M, MUC16,
ZFP36, FN1, SHISA5, TSHZ2, UGT2A1, HSPA5, AGR2, F3, TNC, HLA-DRA, KIAA1324, PIK3R3, SOCS3, IFITM3, CD74,
PSAP, JUND, H3F3B, KITLG, TTC3, ATRX, CSDE1, FAM129A, SERPINF1, CALM1, DDX5, TUBB4B, ACTG1, ALDH1A1,
MCL1, FOSB, CDKN1A, SORL1, CHST9, SYNE2, ATF4, GOLGB1, PRRC2C, S100A6
Ciliated Cells (all)
IFI27, IFI6, HLA-A, SLPI, HSPH1, FKBP5, HSP90AA1, RSPH1, HLA-C, CKB, AHNAK2, IFITM3, CD74, SCO2, LDLRAD1,
WFDC2, PSCA, HLA-F, POLR2I, DHCR24, KRT19, HLA-B, TPPP3, S100P, CYP4B1, HSP90AB1, HSPA8, SCGB2A1, HLA-E,
DYNLL1, ENKUR, ISG15, ERP29, NFE2L1, C1orf194, IFIT1, CFAP53, MAP1A, MT2A, TMSB10, SPR, CCDC113, CFAP36,
OSBPL6, TSPAN1, HSPD1, SERF2, APBB1, ZMYND10, XAF1, CFAP126, LRRC10B, ERG28, NUDC, CDKN1A, FAM183A,
DNAJB2, MAPK8IP1, TUBA1A, METRN, MBOAT7, ARL3, CHCHD10, MGLL, KRT18, MUC15, RIBC2, HOMER2, SAMHD1,
PPDPF, TUBB4B, PTGES3, SPTLC2, PPIL6, IGFBP5, OAZ1, SPATS2L, MORN5, DNAJA1, P4HA2, HSP90B1, C9orf116,
DZIP3, TXLNB, LDLR, CCT3, B2M, TEKT1, SMIM22, IFT22, TSC22D3, METTL7A, CTSD, UQCR11, HSPBP1, GON7,
SLC44A4, CALR, CFAP45, HLA-DRA, SYS1, DBI, ACACA, CAPS, COX7C, CCDC33, SOD1, MS4A8, C3, BTNL9, IPO11, CARS,
NELL2, FXYD3, UFC1, DYNLRB2, UQCR10, SELENOW, FAM216B, HSPB1, RAB36, CALM1, C20orf85, PBXIP1, UPF1,
PSMB8, KRT8, ENPP5, MLEC, ATP6V1D, FTL, MTCH1, TNFSF10, STIP1, COPS9, MAP6, SAA1, TNFAIP8L1, INSIG1,
LINC01765, LRP11, DNAJA4, AC007906.2, NAT14, NLRP1, MPV17L, SQLE, CNPY3, VAMP8, NWD1, CFAP300,
FAM229B, ENSA, SARAF, CAPSL, EFCAB1, BAIAP2, LRTOMT, XBP1, AHSA1, UBA52, UQCRQ, CYB561, PSAP, CSTB,
SHROOM3, WRB, H3F3B, PABPC1, FTH1, CDS1, TCTN1, RIBC1, GDE1, PLEKHB1, IK, DRC1, TSTD1, TMX4, PRDX6, COBL,
UBE2H, DNPH1, ATP5MD, RTN3, LGALS3BP, BAIAP3, ZMAT2, MORN2, EIF1, ALDH3A2, JPT2, WDR34, SPATA18,
C5orf49, PSMC5, H2AFJ, YWHAB, FAU, ZNF106, ATP6V0E1, PDIA6, CLCN3, C9orf135, CNDP2, NME5, TUBA4B, POR,
BDH1, CCDC80, GSTP1, IQCD, STOML3, COX5B, C9orf24, IQGAP1, ICMT, GPR162, SLFN5, MPC2, JTB, RSPH3, HDLBP,
HSBP1, PPP1R16A, CETN2, CCT6A, SLAIN2, COX7B, MX1, CAPNS1, GDI2, ATP5F1E, ARMC3, AC013264.1, HLA-DPA1,
LRPAP1, AES, PSENEN, S100A11, HNRNPC, HLA-DRB1, HLA-DRB5, SLC27A2, FDFT1, ELOB, RUVBL1, PTPRT, CYP2S1,
ARSD, MID1IP1, TUFM, SRGAP3-AS2, THRAP3, AGPAT3, HSPA4, HACD3, DPCD, IDH2, NDUFA4, CST3, OMG, PDCD6IP,
MRFAP1, COX8A, VWA3B, GOLM1, SRP14, KIF9, LRRC6, RAB11FIP1, DNAJB1, PPP1CB, CD24, PTTG1IP, MORF4L2,
SMAP2, P4HTM, RSPH4A, ATP5IF1, CCDC69, RUVBL2, DHRS9, SAP18, ATP6AP1, SKP1, FAM92B, IL5RA, STK33, GPX4,
SSBP4, KTN1, PSMB5, SSB, UBB, CCT7, EFCAB10, GBP3, RTN4, ACO2, DNALI1, SEC62, DAD1, EDF1, KIF5B, CCT2,
SLC9A3R1, TTC25, TSC22D1, CAST, ERICH5, CDHR3, HINT1, C11orf88, C11orf97, TPRG1L, LAP3, PEBP1, ABHD2, AGR2,
CFAP298, CYB5A, SERP1, PARK7, SMIM14, MGAT5, OSCP1, CCNI, LRRC46, AZIN1, PLPP2, TMEM59, PSMD2, ATP2A2,
PKIG, CTNNA1, PTPRN2, CANX, UBL5, TYMP, STAU1, KIF3B, ERGIC3, YWHAE, NUPR1, TAGLN2, COX4I1, RAC1, SF3B2,
LDHB, STRBP, PIFO, ROPN1L, LAMC2, TM9SF2, SNTN, AHNAK, CCT5, HSPA5, UBXN10, UGDH, ARF1, KIF21A, GUK1,
ATXN7L3B, CC2D2A, C4orf3, COX7A2, HNRNPF, ATP5F1B, UCP2, TSPAN3, SPA17, CD164, CHST6, CYSTM1, EIF4G2,
S100A10, VCP, ENAH, ALDH3B1, SRI, IGFBP2, ABLIM1, HDGF, PTPRF, GAPDH, DSTN, EEF2, TMBIM6, CLSTN1, CD9,
C2orf40, CLDN3, ANXA1, TPT1, PKM, CSDE1, LGALS3, SLC7A2, CCDC170, TACSTD2, LCN2, IFT57, KLF6, CALM2, ANXA2,
ZNF664, GNAS, UBC, CIB1, SPAG6, CD59, BASP1, S100A6, TMEM190, MYL6, GADD45B, JUN, ATF3, HSPA1B, ALOX15,
SAA2, NQO1, CTGF
Developing Ciliated Cells
SAA1, HLA-B, KRT19, KRT7, PSCA, IFI6, TMSB4X, HLA-A, SAA2, HLA-E, SAT1, H1F0, MUC1, TXN, SLPI, KRT8, ANXA1,
HLA-C, NCOA7, ACTG1, S100A6, MACC1, NFKBIA, TMSB10, RND3, LCN2, TM4SF1, C15orf48, FTH1, CAPN2, S100P,
ELF3, ACTB, SOD2, FTL, RAB11FIP1, ATP1B1, GAPDH, CD63, WFDC2, HSPA5, MAL2, COX6A1, CD74, UBC, CDKN1A,
TACSTD2, S100A4, COX7A2, SQSTM1, MYL12A, ABLIM1, PSAP, IFI27, TNFAIP3, LGALS3, IRF1, NDUFA4, CAST, HSPB1,
PPP1R15A, HSP90AA1, MYL6, SPTBN1, BPIFA1, KLF6, GBP1, KRT23, ANXA2, PKM, ATF3, KRT18, GDF15, PPP1CB,
TPM4, HSP90B1, CLINT1, S100A14, COX6C, PABPC1, S100A2, HSP90AB1, CSTB, F3, AQP3, COX7B, HEBP2, SPATS2L,
UQCRQ, YWHAZ, TPT1, CXCL8, CD55, ATP5MG, FOS, EIF1, ATP5F1E, HES1, COX4I1, PARP14, SERF2, UQCR11,
S100A10, ITGB8, CLDN4, H3F3B, COX7C, CANX, EZR, DUSP1, SKP1, ALCAM, HSPA8, HSPA1A, KRT17, JUN, DSTN,
S100A11, GLUL, YWHAE, DNAJB1, AGR2, CALM2, HSPH1, SARSCoV2-3prime, CALM1
Goblet Cell (all)
IFI27, S100A9, PSCA, TSPAN1, AQP5, KRT19, CYP4B1, S100A8, S100P, HLA-A, IFITM3, SERPINB3, GSTP1, VMO1, CTSD,
SERF2, F3, DHCR24, MX1, HLA-B, PI3, CAPNS1, GAPDH, HLA-C, XBP1, EPAS1, GOLM1, MUC1, KRT7, ASRGL1, LYPD2,
VAMP8, TMSB10, HLA-E, AKR1A1, CTSB, P4HB, PRSS23, RARRES3, SLC6A14, AGR2, FUT2, ATP1B1, LGALS3BP, OAZ1,
CEACAM6, IFI6, B2M, HSPA8, TACSTD2, DYNLL1, IFITM2, ANXA2, PKM, CALR, MYL6, HSP90AB1, PRDX1, STARD10,
WFDC2, SLC9A3R1, ATP5F1B, HSPA5, FXYD3, ENO1, HDLBP, SYNGR2, HLA-DRB5, SCD, TSPAN13, GRN, UQCRQ,
KRT18, ACTG1, TPI1, CCDC69, KCNE3, COX7A2, TMBIM6, S100A14, PSAP, S100A16, GUK1, CALM1, FBP1, SORD, LCN2,
COX5B, FAM3D, UBC, DCXR, PSMB8, CAPN2, GSTK1, SSR4, CD24, UBB, TSPO, PRDX5, ACSL3, STEAP4, CSTB, LY6E,
LDHA, BLVRB, ARF1, JTB, TUBB4B, ST6GAL1, B4GALT5, EDF1, SLPI, ALPL, SAA1, COX6B1, KRT4, SELENBP1, VCP,
YWHAB, ELOB, ALDH1A1, ATP1A1, FAM107B, NANS, TUFM, ERP29, ENSA, MAL2, TUBB, CD74, SLC44A4, PGK1, LRP10,
NR2F6, ANXA11, MDH2, NQO1, RARRES1, GPX2, S100A6, UBA52, HLA-DRB1, UQCR10, ADH1C, NDUFB2, CD82, FAU,
MSLN, PDIA6, PGD, A4GALT, PSMB6, COX7C, NDUFA4, UPF1, S100A10, GLUL, COX5A, RPN2, SDC1, DUOX2, OAS1,
TKT, TALDO1, ALDH3A1, TSTA3, CLDN4, FTL, ATP5F1A, ACTB, CCDC80, CD36, ASCC2, SEL1L3, S100A11, ECHS1, ST14,
GSN, CFL1, FDFT1, NDUFA6, COX4I1, ECH1, RAB25, CAPN1, XAF1, PSMA7, CAP1, CST3, LDLR, PSME2, DBI, RNH1,
RACK1, ABLIM1, FBXW5, HSPB1, UQCRH, S100A4, MSMB, PDIA4, RHOC, CYP2F1, PPDPF, CANX, RAB10, CSDE1, ASS1,
PDIA3, HNRNPC, LMAN2, PSMD8, GDI2, NAPRT, SFN, TUBA1B, PERP, KIF5B, CDK2AP2, MLEC, DEGS2, NDUFB10,
PSMB4, PLAC8, GCNT3, ARPC2, ANAPC11, COX8A, HLA-F, EEF1D, H3F3B, HSP90AA1, BRK1, UQCR11, CSTA, EZR, CYC1,
HSP90B1, PPA1, APRT, SREBF1, TMED9, C19orf33, ARHGDIB, NDUFS6, C3, MISP, AQP3, PTTG1IP, TMED3, BCAS1,
BACE2, ATP6V0E1, PHB, CREB3L2, GALNT7, ATP5MG, PSME1, PCBP1, EIF4G2, ALCAM, ATP5ME, LGALS3, PLPP2,
TXN2, UBL5, SOD1, CEACAM5, NDUFS7, PTP4A2, NDUFS5, SLC2A1, CXCL17, KRT8, SCAMP2, ATP6V1F, GNG5, CNDP2,
PHPT1, EIF3K, LDHB, ATP5F1E, RHOA, MCL1, NCOA4, CCNI, HSBP1, CLTC, HINT1, SPDEF, IFITM1, CUTA, EIF1, PSMB5,
RAC1, ATP5MC3, DYNLL2, DNAJB1, CCT5, CHP1, PSMB1, RBM47, SNRPD2, SEC31A, PPP1CA, TRIP6, ALDH3A2, DAD1,
CREB3L1, TAGLN2, EIF4G1, ECI1, RDH10, ASAH1, NDUFB7, SMIM14, FDPS, CKAP4, ELF3, C19orf53, TMEM59, DAP,
LAMTOR5, MRFAP1, SSR1, PIK3R3, FUT3, ATP6V0B, MUC20, CTSH, SMDT1, CCND1, CDC42, PSMC5, JPT1, SERP1,
TAPBP, COPB2, SHISA5, GLRX, VWA1, CLTA, DUSP1, B3GNT3, SDF4, RAB37, PSMC3, RAB2A, MYL12B, SPTLC2,
CTNNA1, HLA-DRA, CCT3, UQCRC1, ATP5MPL, TUBA1C, SPINT2, ARL6IP4, NME1, SLC15A2, MVP, COPZ1, SAP18, EEF2,
COMT, RASSF7, SMIM22, SARAF, DSTN, ST6GALNAC1, ICMT, CIB1, PPIB, SERINC2, PSMD2, HADHA, SLC25A5, EPS8L1,
BAG1, CBR1, EIF3I, KDELR2, FUT6, SPINK5, EID1, PABPC1, GPAA1, HNRNPA2B1, COX6A1, C1orf43, HSPH1, XRCC6,
YBX1, EIF6, SRI, HNRNPM, COX7B, TMPRSS4, SSNA1, TSPAN3, ISG15, REX1BD, OAS2, ACTN4, LAPTM4A, POLR2L,
ATF4, ARF4, ATP5MC2, GGT6, CLTB, ACO2, C1orf122, COPS9, CALM3, SH3BGRL3, NDRG2, UGP2, GLTP, FAM129A,
UBXN4, PSMB9, RNF10, TST, TMEM219, SEC61A1, OST4, RAB7A, C1orf116, PPCS, GPI, TJP3, PSMB7, LITAF, UBA1,
CFB, SRP14, VPS28, EPRS, ARPC3, TRIR, VSIG2, APOL6, NAA38, FCGBP, CD151, FLNB, ROMO1, NDUFB9, F11R,
RAB11A, IFI16, FOS, PSMA4, ATP6V1G1, PFDN5, FAM83A, ZNHIT1, TMEM125, TMEM160, ATP5PD, GOLGA2, SNX17,
FKBP4, ARPC5, FAM96B, ARL1, PDXK, SEC13, PIGT, YWHAZ, AK2, MIEN1, TRIM29, JPT2, SF3B2, CTSS, SMAGP, HDGF,
STAT1, HSPA1B, DUOXA2, NUCB2, CALM2, XRCC5, SAMHD1, KPNB1, MTPN, NDUFS2, APLP2, SELENOH, LAPTM4B,
ATP5IF1, AURKAIP1, PTMA, C12orf57, NDUFAB1, PSMD1, TMEM54, H2AFZ, PRDX6, JUN, AP2M1, FASN, HSPA4,
COPA, VDAC1, WDR83OS, INSIG1, JUND, GCHFR, ITM2B, ATP5MD, BAG6, DYNLT1, PRSS8, CHMP3, RDX, TIMM13,
EPHX1, FAM120A, NDUFA13, CAPN13, PARK7, PNKD, TNFSF10, DHRS3, VILL, CD55, PTGES, AUP1, CD59, GNB1,
STUB1, BST2, TPM3, PSMB3, SLC25A3, FKBP2, NDUFA1, IL13RA1, TMED10, DTX2, DTX4, GSPT1, UBE2L3, COX14,
TAX1BP1, RAN, HIGD2A, RBX1, GALE, ATP6AP1, NDUFA2, OAS3, PROM2, BRI3, CCT2, MTDH, DNAJA1, CPD, QSOX1,
CAST, CHMP2A, PUF60, GNE, NUCKS1, RALBP1, HSPA9, LRRC59, PDAP1, AHCY, AVPI1, ENC1, CXCL16, TM9SF3,
C19orf70, SF3B5, FTH1, COA3, LRG1, GNS, HSPA1A, VAMP5, SNU13, MYL12A, COX6C, CA12, IQGAP1, EFCAB14,
DTX3L, MTCH1, SLC25A39, NCL, NUCB1, DNAJB2, STAT3, MAGED1, GNG12, GADD45GIP1, GMDS, ALKBH7, NAXE,
AES, CALU, TMEM141, SLC39A7, BTF3, TAF10, TXNDC17, TPM4, AP2S1, MEA1, AKR1C1, UBE2L6, TXNL4A, KIAA0513,
CERS6, HACD3, ANXA4, NEDD8, B4GALT1, SQSTM1, RNF181, PYGL, LRP11, S100A13, POMP, IRAK1, NT5C2, SLC1A5,
RNF7, CXXC5, PLIN3, GLG1, NDUFB4, C6orf132, SEC11C, VSIR, TM7SF2, FLOT1, FIS1, ACTR2, EIF4A2, GM2A, SCNN1A,
PRELID1, MAOA, MDH1, NHP2, ID1, CSNK1A1, IFI44L, STIP1, SEC61B, SNX3, SCPEP1, CENPX, SLC31A1, PLEKHS1,
PPP1CB, SLC27A2, RNPEPL1, EBP, SERINC3, CCT6A, HLA-DPA1, PPM1G, CCPG1, CD81, AHCYL1, PLXNB2, SSU72,
MSMO1, HEBP2, KDELR1, DCAF7, TERF2IP, SURF4, TXN, CAPZB, MGST2, EMC4, TSC22D1, C6orf106, TCIRG1, ATP5PO,
TAP1, KRT24, PARP9, GSR, RAB1B, SPRR3, FBLN1, CYB5A, CSRP1, FAM129B, IRF7, GNAS, IDH2, CTSA, PPL, OTUB1,
SRPRB, HERC6, EI24, PTPRF, RPN1, TXNL1, NDFIP1, COPS6, EIF5, UFC1, JUP, GHITM, RHBDL2, HTATIP2, ADAR, RRBP1,
AZGP1, MPC2, CHCHD10, AKR1B10, SELENOW, CYB561, PIGR, TCEA3, ZNF185, PDZK1IP1, EIF2AK1, C6orf62, POLR2F,
ARCN1, ALDH9A1, SLC16A9, CCT7, DDX6, CHCHD2, TMEM30A, CD63, DNPH1, SLC44A2, GOT2, ADI1, YIF1A, POLR21,
GDF15, PSMD13, ILF2, RAB11FIP1, PPP1CC, SOX2, YWHAE, H2AFY, SCARB2, FAM114A1, ERLIN1, GPX4, SPAG7,
ATP5PF, YIPF3, RETSAT, ANAPC16, HNRNPF, TXNIP, RSL1D1, CYR61, SLC25A11, TCP1, TOP1, SERBP1, MXD4, VAPA,
SCEL, KLF6, PCYOX1, OS9, RAB1A, CRACR2B, CYB5R3, ACAA1, WDR1, SKP1, RTRAF, TMEM147, BDH1, RND3, LASP1,
GDE1, PQLC1, CRYM, EMP2, RTN4, ERBB2, RAB8A, TMEM258, PLPP5, GBP3, TMED2, PTAFR, PLEKHB2, EFEMP1,
DDOST, CAPN5, SET, SUMF2, ATP5F1C, PTOV1, UXT, HSD17B10, HSPD1, STT3A, NOP10, VCL, MBOAT2, TRIOBP,
LSM7, PSMC2, RAB3D, MYH14, MANF, FLII, KARS, PSMB2, TMEM165, CDKN1A, ALG3, SLC16A3, ALDH3B2, POR,
GPT2, PABPC4, PGLS, PSMD4, MYDGF, EFHD2, ARHGAP1, CTSC, RDH11, COPE, EIF2AK2, UBE2D3, RTN3, MZT2B,
GPS1, SUCLG1, TIMM17B, NAGK, HM13, CNPY3, PSMC4, HK1, HNRNPU, SSRP1, YME1L1, MPZL1, FOXA1, SERINC1,
TMEM208, KIF1C, VPS25, RNPEP, EMC10, RAI14, SDHB, NFE2L1, PMVK, TMEM205, ASPH, TRIM16, CNN2, CLINT1,
CMAS, GFPT1, ERH, MFSD1, ATP6V1E1, SERTAD1, HAX1, CRK, CLIC6, ADD3, FARSA, GDPD3, MON1B, KRT10,
PPP1R11, FAM32A, ATP5PB, IMP3, YWHAG, AHSA1, SCP2, KHDRBS1, COPG1, PDLIM1, ARPC1B, TMEM9B, UQCRB,
CLDN7, ADGRG1, UBE2N, 2-Sep, SLC25A1, CAPZA1, NUDC, DNAJC3, PSMA3, GRB2, PAQR4, GNA15, GTF2F1, CANT1,
PLPBP, PSENEN, TTC3, SYPL1, HSPE1, APEH, PPP1R15A, AHR, ACBD3, PA2G4, SYAP1, HLA-G, STS, ARPC4, ACTR3,
ATF6, TFG, RAB5IF, LSM4, DDRGK1, TMCO1, PRKAR1A, SLIRP, RER1, MLPH, TM9SF2, ACOT13, TCTA, LLGL2, NONO,
GABRP, CORO2A, EIF3G, NDUFC1, ERG28, GAA, EIF5A, SH2D4A, POLDIP2, IPO7, SLC3A2, NIPAL3, SSR2, PTBP3,
DYNLRB1, ADIPOR2, RAB14, UROS, SELENOF, SSR3, SRSF3, LAMP1, CHCHD1, PDHB, BTG1, DSG2, GNPNAT1, TGOLN2,
ADIPOR1, PDLIM5, TSPAN6, PAPOLA, GMPPB, SND1, PYGB, PRDX2, PAM, FHL2, PLEKHJ1, SEC63, SLC35A4, SRP72,
PRDX4, PDCD6IP, ARRB2, SAR1B, AP1M2, HYOU1, CPPED1, POLR2E, CNBP, FUCA1, DDX49, RSU1, SLC39A11, SUN2,
PHACTR4, MAP2K2, SLC35C1, RNF187, NDUFA8, CD9, LAMTOR1, TSR3, DNAJC15, IDI1, LGI1, ALDH2, TRAM1,
ATP6AP2, ATP6V1D, SCAP, LPCAT3, BCCIP, CCNO, PARP1, MORF4L2, PRR15, ALOX15, FAM98A, MLF2, VWASA, NARS,
STOML2, NDUFS3, ATP5MC1, ARHGDIA, CCT8, MCRIP2, TMBIM1, BSG, TMX2, PFKP, TIMMDC1, PPP2CA, ATRAID,
ARF6, PTGES3, NSFL1C, EPHA2, OCIAD1, EIF3H, KLF5, UGCG, GNAI1, BAG3, CDH1, CYP4X1, SLC38A10, TMEM87A,
DCTN2, SLC40A1, SNRPC, BSPRY, XIAP, STOM, EIF4EBP2, AP3D1, CD46, AC104126.1, SGPL1, ANAPC13, TMC4,
SLC52A2, IL1RN, TNFRSF21, LGALS8, HSBP1L1, HNRNPAB, WARS, DMAC1, CCT4, ME1, G3BP2, HMOX2, CWC15,
HMGA1, LMNA, KPNA6, GRHPR, BUD31, NDUFA12, FAM120AOS, HS3ST1, ANXA5, PTP4A1, PQBP1, CTR9, CBX6,
LONP2, LIMK2, SRPRA, TIMM8B, MYH9, DDB1, MPST, MATR3, MARCKS, DHCR7, NDUFS8, PYURF, NRDC, EMC6,
ZMAT2, RAB5B, BCAP31, NME3, EIF5B, FUCA2, LAMTOR4, MACC1, PRPF40A, GSS, ZBTB38, DNAJA4, NENF,
NIPSNAP1, STT3B, NAP1L4, THEM6, TM7SF3, DIAPH1, TYMP, LINC01133, PLPP1, BRD2, IMMT, RAD23A, BCR, DUT,
QARS, MGAT1, SYNCRIP, DUSP23, YY1, CD164, NPDC1, RABAC1, NDUFB3, WNT7B, CHMP1A, UBA7, YIPF5, LRPAP1,
WASL, ARSD, ADAM9, LMAN1, ERGIC3, UAP1, SCO2, STARD7, ILVBL, MMAB, METTL9, NDUFB8, HMGN1, DDX1,
GTF3A, NOLC1, GORASP2, RNF5, CYP2J2, SMC4, SPCS1, ATXN7L3B, CREB3, RNF20, PPP1R7, EXPH5, TRIM26, CRELD2,
ACSL1, CTNNBIP1, ARHGAP35, C19orf24, C4orf3, TTC1, KTN1, TP5313, CTTN, ELL2, TRMT112, SLC4A4, SPCS3, UQCC2,
IK, WASF2, NCCRP1, NFKBIA, SNF8, SCAND1, MVD, TEX264, ACTR1B, ACLY, EDEM3, PSMA1, NECTIN2, CRCP, EMC3,
DAZAP2, RABL6, SEC11A, PSMA5, PDCD6, HDAC1, RNF145, COPB1, UBQLN1, CHL1, IFNAR1, KIAA0319L, ECHDC1,
DEDD2, LARS, TMED1, SMARCC1, MAGT1, ATIC, TPD52L1, ORMDL2, PLSCR1, COQ4, CLIC1, NACA, EIF4B, SON, COQ9,
NEBL, NSF, OSBP, NDUFB11, CTDSPL, RALY, RNF114, FKBP1A, RMDN3, PEBP1, EIF3A, GNAQ, LSM3, TMEM167A,
MESD, ANP32B, BCL2L1, UBE2D2, SELENOK, ACACA, LAMB2, BCL2L15, TRNP1, CASP3, TRIM25, USP22, TMBIM4,
SLC35E1, GGCT, MPV17L, FKBP3, PITX1, METAP2, CHST6, IDH1, GOLGA7, PTPRU, PRMT2, DPM3, MED8, EEF1B2,
TSTD1, RGL2, HLA-DMA, DUSP3, DNAJC8, NMRAL1, CASP4, PSMD11, PTBP1, CMPK1, SEC24C, TMEM30B, ARFIP1,
LAP3, SARS, AIP, MARS, SCAMP3, MFN2, PSMD14, EBNA1BP2, RTF2, PSMD7, DFFA, AGRN, ACTR1A, RBM8A,
GOLPH3L, BAZ1B, TSPYL1, CACFD1, MPZL2, PDHA1, PAFAH1B3, SLC12A2, ERLEC1, RBCK1, SZRD1, SURF1, DAP3,
DCTPP1, MYO1B, CYCS, YIPF6, NDUFV2, CNP, ERMP1, UBXN6, ESRP1, RXRA, NUDT5, TMEM159, ATOX1, ARL8B, PJA2,
PRPF6, AP1B1, ERLIN2, SELENOS, G3BP1, ATG101, C1QBP, NUDT8, ALDH3B1, EIF3L, MUC21, PHB2, SDF2L1, ETFB,
VGLL1, ARPC5L, NDUFS1, TMEM179B, LGALS9, CHTOP, DNTTIP2, GOLPH3, SUMO2, ELOF1, CDC37, RAD23B, ALKBH5,
TMSB4X, BAD, BPIFB1, DGCR2, METRNL, PPIC, ATP5MF, LRRC8A, FIBP, ZNF106, TMEM263, IFIH1, ETF1, TBC1D9B,
ADIRF, MORF4L1, TUBA4A, HNRNPH3, SYT8, UGT2A1, AMOTL2, SPG21, SYNGR1, UHMK1, GOLGB1, SMC1A, PPT1,
MECOM, ERICH5, REEP5, DENR, GIPC1, MAPKAP1, AGL, HP1BP3, TMX4, ZCRB1, SPATS2L, EIF3D, TMEM123, PEA15,
FYCO1, PRKAR2A, EBAG9, SPTY2D1, DSP, CNIH4, PRRC1, SLC50A1, PREB, TMOD3, CFDP1, CTCF, MMADHC, NDUFB6,
UBXN1, DUSP6, FBL, ARL4D, SAR1A, MPC1, THRAP3, NOSIP, ALAD, SCRIB, ANXA7, GARS, CNN3, TPRG1L, NDUFB5,
PSME3, PRDX3, CAB39, ZFP36, PAFAH1B1, MESP1, POLR2K, HIST1H2AC, TBL1XR1, FAM173A, UQCC3, GNA11,
TRIM14, PFN1, ADGRF1, CDK10, HADHB, PSMD3, ERO1A, VTI1B, 11-Sep, LYN, CD47, SPINT1-AS1, HINT2, GADD45B,
RAB5A, SDR16C5, CFH, RAB27B, MCU, CHMP4B, UTP14C, G6PD, SDC4, TMEM50B, ATP2A2, ALAS1, YBX3, GALNT1,
IP6K2, ATP1B3, RHOV, AP3B1, AGPAT3, PRRC2B, SCYL1, NDUFB1, RSRP1, DHX29, XPO1, TMEM259, TOX4, MYO1C,
GTF2A2, MAPK13, SYTL1, RBBP9, ZNFX1, SNRPD3, EPN3, MGST1, ESRRA, EIF2S1, MTIF3, VAMP3, CMTM6, ZDHHC3,
HERPUD1, APEX1, PON2, CCDC186, IDE, ATP10B, EIF3J, DBNL, FAM83E, TBC1D5, EIF2S2, DHX9, PCDH1, ATP8B1,
ZDHHC5, SGPP2, BROX, SH3GLB1, CISD3, CLCN3, LYPD6B, SMARCD2, HDAC2, ENTPD4, CHMP1B, ATXN10, SPTBN1,
GLUD1, FAM83D, SYNJ2BP, LMO4, MTCH2, NAA20, PGRMC1, SEC23B, TSPAN14, REXO2, CAPRIN1, IER2, ARF3,
UBE2H, UBE2K, HMGN3, CXADR, NPC2, ZMPSTE24, AARS, WDR13, EMC7, SMARCA5, TES, TOB2, SLC16A7, VGLL4,
BTN3A2, HSPA6, GNB2, GOLGA4, TACC2, TRIM44, WIPF2, FBXO34, TMF1, ECPAS, TMCO4, 7-Sep, CDS1, NR2F2,
KIAA1191, ACOX1, CCDC47, SMG7, MTURN, CHMP5, EPB41L1, EIF4E2, TRA2B, GPD1L, NAV1, UBE4A, STX10, ABHD2,
SREBF2, ZFR, PPM1K, EIF3B, LIPH, IL6ST, DDX21, KIAA0100, CLSTN1, MAPRE1, LIMA1, GSTA1, VPS35, TRIP11,
SERPINF1, PTGR1, PADI1, ANP32A, ZNF652, RNASE1, CARD19, UQCRC2, NPM1, AZIN1, CAMK1D, CRYBG1, SEPHS2,
UBE2R2, NUP50, FOSB, FAM84A, PAPSS1, ANTXR1, TMEM33, AMD1, MAN1A2, CHURC1, EIF2A, BNIP3L, MID1IP1,
SOX4, FAM210B, UNC93B1, H2AFJ, SEC24D, TRIM8, STK24, EIF3M, CHP2, HNRNPK, CD2AP, DHRS9, STAU1, DNAJC21,
ITPKC, CADPS2, RRAGD, LAMP2, LSG1, HPGD, GABARAPL2, CDCP1, SPINT1, KDM5B, TAF7, SERPINB6, NIPAL2, ATP9A,
CCDC12, NSD3, CNOT1, ID2, NFE2L2, SSB, GPRC5C, DNAJC10, ARL6IP5, ELOA, TNIP1, HES1, DUS1L, VPS37B, SP1,
TMC5, TLN1, TOMM70, SCAF11, SCIN, NAA50, CAND1, HOMER2, DDR1, GOLGA3, RAD21, COL4A3BP, NDUFV1,
ABCF1, MKNK2, MDK, SMARCA4, CBX5, FNDC3B, PRRC2C, MDM2, MAFF, EYA2, ZC3H15, CTBP2, JUNB, ISCU, DLG3,
ERN2, HTATSF1, EIF4H, UBE2Q1, CEBPD, ADAM10, FGD5-AS1, SIVA1, ATP6V1A, PPM1L, HSD17B4, DYNC1H1,
DNAJC1, JAK1, TOMM7, PRKAR2B, HOOK1, NORAD, TMEM50A, CPNE3, RABGAP1L, ZBTB7C, LY6D, SSBP1, TRPM4,
TP53I11, CCDC6, RCC2, TTC9, SH3PXD2A, SRP54, SIX3, LYNX1, MKRN1, GALNT3, UBE2J1, IQGAP2, DDX24, SLFN5,
ELOVL5, ANPEP, SEC62, KLF3, ZKSCAN1, TUBB2A, PPP2R2A, SGK1, SAMD9, SRSF7, ELF1, STK39, SERPINB1, GGNBP2,
APOL1, TOMM20, TAF15, KIAA1522, MIA3, LARP7, LARP4B, NOL7, CTNND1, AKR1C3, USO1, ZMYND8, IBTK,
C11orf58, ARGLU1, TPT1, ZFAS1, LARP1, NEDD4L, IRF2BP2, CHD3, SLK, ARID5B, WNK1, NFIA, OPTN, KRT6A, BMP3,
OAT, ADH7, IRAK3, ATF3, CXCL1, MYO5B, MGLL, SASH1, HNRNPUL1, HNRNPR, BBX, CDC42BPA, METTL7A, PRPF38B,
PDCD4, KRT23, ATF7IP, TFCP2L1, H6PD, SOCS3, ZC3H13, MUC4, ANKRD17, ARFGEF3, TRIP12, ECE1, FAT1, CIRBP,
ITGB1, IRF1, MAGED2, ANXA1, TNFAIP3, KIF13B, SRD5A3, TC2N, SECISBP2L, LPP, TCF25, SLC25A6, LMO7, STIM2,
EPS8L2, TRIM2, KLF4, CHD4, HUWE1, EIF4G3, DDX5, TRIM56, KIF21A, SF3B1, CYBA, KRT13, NFAT5, SAA2, DDIT4,
MUC16, ACADVL, PTPN13, PARP14, DDX17, HECTD1, EGR1, BHLHE40, EHF, CYP2A13, GPRC5A, MT2A, AKR1C2
Ionocytes
IFI27
Macrophages (all)
FTH1, FTL, BEST1, PLIN2, CD63, SRGN, CTSL, FCER1G, SERF2
Secretory Cells (all)
MSMB, IFI6
Squamous Cells (all)
SAA1, IFI6, PDZK1IP1, IFI27, S100A9, AGR2, TMSB10, SAA2, GLUL, TYMP, C15orf48, FTL, FCGBP, TXNIP, S100A8,
TSPAN1, CXCL17, VMO1, KRT16, KATNBL1, C3, DSG2, LCN2, TMEM160, BEST1, ISG15, SERF2, IDO1
AZGP1 high Goblet Cells
PSCA, S100A9, SERPINB3, IFI27, CYP4B1, TSPAN1, EPAS1, AQP5, SERF2, KRT19, DHCR24, RARRES1, CTSD, IFITM3,
HSP90AA1, CAPNS1, HDLBP, HSP90AB1, FOS, XBP1, HSPA8, FAM3D, PRSS23, MX1, P4HB, ATP1A1, VMO1, GSTP1,
COX5B, S100P, CD24, GAPDH, RACK1, F3, HLA-A, LGALS3BP, S100A8, TSPAN13, ADH1C, SYNGR2, NDUFA4, UBB,
ASRGL1, STARD10, SLC9A3R1, FXYD3, FAM129A, COX6B1, UBC, IFITM2, CALR, OAZ1, SCD, ACSL3, FAU, B4GALT5, IFI6,
CCDC69, CYP2F1, BLVRB, HSPB1, HSPA1B, CCDC80, ATP5F1B, STEAP4, JUN, FUT2, PERP, TMBIM6, DYNLL1, AGR2,
TUBB4B, PKM, ALDH3A1, MSLN, PABPC1, PLEKHS1, GOLM1, CXCL17, LYPD2, ASCC2, NQO1, CSTB, DCXR, HLA-E,
ST6GAL1, LDLR, KIF5B, FDFT1, ENSA, UQCRQ, SELENBP1, HSPA5, YWHAB, NUCKS1, CAPN13, CTSB, TMSB10, COX7C,
LY6E, AKR1A1, RAB10, EEF2, UPF1, SORD, TUBB, VAMP8, ATP5F1A, CANX, ECHS1, ALDH3A2, UBA52, RBM47, TKT,
HNRNPC, DBI, EDF1, TMPRSS4, HLA-DRB5, PRDX5, KRT18, ARF1, TPI1, ANXA2, SSR4, KRT8, NDUFA6, GABRP, GRN,
SDC1, GOLGA4, PDIA6, UQCR11, NDUFB10, CREB3L2, PIK3R3, CREB3L1, TUFM, PDIA3, SOX2, A4GALT, NDUFB2,
SNRPD2, ATP5ME, ERP29, PTPRF, JTB, TXNIP, PGK1, GUK1, SLC25A5, NDUFS5, PSAP, ATP5F1E, NUCB2, S100A6,
RNH1, MLEC, CCNI, HLA-B, CD36, HLA-C, GALNT7, CNDP2, YBX1, SRI, RPN2, KRT7, ATP1B1, BAG1, EIF4G1, NANS,
SLC15A2, EIF4G2, MSMB, SREBF1, HSP90B1, DEGS2, FBP1, NR2F6, TSTA3, TMED9, SSR1, XAF1, JUND, CAPN1, NCOA4,
HSPH1, CALM2, UQCR10, FOSB, PPIB, TCEA3, NDUFS7, FLNB, KCNE3, SERP1, CHL1, DTX4, ASS1, GSTK1, TOP1, GDI2,
RAB25, ECH1, TAGLN2, ENO1, EIF4A2, TTC3, CDC42, OST4, CPD, COX7B, C3, FDPS, SARAF, FAM107B, KTN1, COX7A2,
MDH2, SMDT1, HINT1, TUBA1B, GSTA1, GLG1, SEC61A1, CUTA, UBXN4, ATP5MC2, ARL1, GPX2, HSPA1A, GOLGB1,
CD74, UQCRH, NCL, CST3, TSPO, GLUL, PPDPF, COX8A, UBA1, EPRS, COMT, NDUFB7, CDK2AP2, H3F3B, CFL1,
ST6GALNAC1, SCAMP2, SEL1L3
AZGP1 SCGB3A1 LTF high Goblet Cells
IFI6, IFI27, RARRES3, HLA-DRB5, SAA1, SCD, HLA-A, CALR
BEST4 high Cilia high Ciliated Cells
SCO2, BTNL9, WDR6, HLA-F, CFAP70, WDR27
Cilia high Ciliated Cells
IFI6, IFI27, HLA-A, HSPH1, HLA-B, HLA-C, CD74
Early Response FOXJ1 high Ciliated Cells
IFI6, FKBP5, AHNAK2, HLA-A, IFI27, SLPI, SCO2, HOMER2, WFDC2, PSCA, CKB, TSC22D3, S100P, SPON2, SCNN1A,
DHCR24, KRT19, SLC6A8, NFE2L1, PDK4, ID2, APBB1, CFD, OSBPL6, SAA1, HSPA8, INSIG1, VSTM2L, METRN, SPR,
SOCS3, MUC16, ID1, LDLRAD1, MUC15, S100A8, TFCP2L1, FHAD1, ZBTB16, XIST, CAPN2, SCGB2A1, S100A9, COBL,
HLA-F, PFKFB3, ERP29, ID4, TPPP3, PDE4DIP, LDLR, ARSD, CALR, SORD, CEP126, SGK1, HLA-C, CYP4B1, HMGCR, PIM3,
RSPH1, C1orf194, PQLC1, CD74, DNAJB1, MT2A, IFITM3, TM7SF3, HLA-E, MAFF, RIBC2, DNAJB2, LINC01765, PRKX,
ICAM1, ID3, ALDH3A2, NELL2, PAQR8, PKIB, AHNAK, YBX3, HLA-B, TXNIP, ST6GAL1, LPIN1, HMGB2, TMSB10, NWD1,
CCDC33, HSPB1, CD99, LBH, XAF1, LCN2, NBL1, PPL, CDKN1A, DSG2, PPDPF, PPP1CB, ERBB4, NFKBIA, PRPF38B,
SLC43A2, CDHR3, TFF3, TMEM50B, P4HA2, MGLL, SPTLC2, CTSD, BAIAP3, CRLF1, METTL7A, ZBTB7A, POLR2I,
DYNLRB2, OGA, KLF10, SLC44A4, ENKUR, MS4A8, MTCH1, PTP4A1, FTL, BAG3, HSPH1, FLNB, XBP1, ENPP5, TEKT1,
SLC23A1, GADD45B, CCDC113, ATP6V1D, UBA1, CFAP53, RNF145, CCDC151, HP1BP3, BCO2, CDKN2AIP, ANXA4, VILL,
FAU, C3, TNFSF10, RHOB, CLU, KRT7, LRP11, CFAP126, LRPAP1, WEE1, FAM183A, PSAP, PPIL6, MAPK8IP1, SELENOP,
RTN3, BPIFB1, DEDD2, JUN, RAB11FIP1, RALGAPA2, CCDC24, PBXIP1, CFAP45, LRRC10B, OAZ1, RSRP1, SLC25A36,
RIBC1, NFIC, KIAA1841, PABPC1, CCNL1, SMC1A, DYNLL1, IDI1, CLCN3, LPIN2, INHBB, SUMF2, QSOX1, ABHD2,
SRD5A2, CCDC80, KRT18, CNPY3, FANK1, IER5, SQLE, RHOU, TOB2, TRIB1, CYR61, PPP3CA, KTN1, LGALS3BP, DNAJA4,
BHLHE40, SHROOM3, SUN1, KIAA1671, MPC2, IQGAP2, MLEC, CFAP36, TUBA4B, IFT140, KIAA2012, EFCAB1, MTSS1
FOXJ1 high Ciliated Cells
IFI6, IFI27, AHNAK2, FKBP5, HLA-A, SCO2, RSPH1, HSPH1, LDLRAD1, PSCA, HSP90AA1, OSBPL6, CKB, HLA-C, CYP4B1,
DHCR24, MAP1A, ACACA, IGFBP5, POLR21, NFE2L1, LDLR, MUC16, TXLNB, SCGB2A1, IFITM3, CCDC113, ERG28, HLA-
B, CFAP53, MAPK8IP1, ERP29, TPPP3, MSMO1, BAIAP3, APBB1, ENKUR, HSP90AB1, DZIP3, HOMER2, HLA-E, CDKN1A,
DNAJB2, HSPA8, IPO11, SQLE, DYNLL1, C1orf194, HSPD1, HLA-F, CD74, PBXIP1, RIBC2, SLPI, ARL3, NUDC, KRT19,
SPTLC2, ZMYND10, NELL2, RETREG1, CFAP36, FHAD1, RSPH3, ENPP5, LRRC10B, LRRC6, GON7, TSPAN1, MGLL,
MPV17L, TCTN1, HSP90B1, SAMHD1, PTPRT, COBL, RAB36, SPATS2L, MAP6, EFCAB1, FAM183A, CFAP126, CFAP45,
TUBA1A, SERF2, TMX4, AC007906.2, CEP126, SOD1, HSPBP1, CCT3, KRT18, P4HTM, IQCD, TEKT1, FAM216B, VWA3B,
MGAT5, NWD1, PPIL6, MORN5, CCDC180, UPF1, MUC15, DRC1, FTH1, MS4A8, UGT2A1, LRP11, C9orf116, SARAF,
DNAJB1, TMSB10, PTGES3, ZNF106, IFT22, IL5RA, TNFAIP8L1, UFC1, CYB561, CDHR3, SPATA18, CALM1, UCHL1,
HDLBP, CC2D2A, WFDC2, S100P, KIF21A, DNAJA1, OAZ1, DNAJA4, JPT2, METTL7A, UBE2H, ATP2A2, C20orf85,
CCDC80, PSAP, CAPS, TUBB4B
MUC5AC high Goblet Cells
XAF1, IFI27
Interferon Responsive Ciliated Cells
IFI6, IFI27, POLR21, HLA-A
ITGAX high Macrophages
FTL, FTH1, BEST1, CD63
SCGB1A1 high Goblet Cells
IFI27, S100P, CYP4B1, XAF1
SPRR2D high Squamous Cells
LCN2, SAA1, FTH1, FTL, PDZK1IP1, PSCA, BEST1, TMEM160, ELOB
CCL5 high Squamous Cells
S100A9, S100P, SLPI, PSCA, KRT24, S100A8, SERPINB3, TMSB10, LCN2, IFI27, KRT19, MUC1, WFDC2, SPRR1B, AGR2,
VMO1, SPRR3, SAA1, ANXA1, C15orf48, HLA-A, IFI6, KRT7, LGALS3, TSPAN1, GLUL, B2M, HLA-B
VEGFA high Squamous Cells
IFI6, IFITM3, ISG15, XAF1, IFIT3, IFI27, IFIT1, TYMP,
Table 3B. Expressed in Control WHO 0 individuals
Ciliated Cells (all)
TBC1D8, TNFAIP2, FRMPD2, RBM3, DLEC1, LRRC74B, PROM1, CROCC2, ABCA13, ANKRD36C, GSTA2, NEAT1
Developing Ciliated Cells
DNAH5, TMEM190, GSTA2
Goblet Cell (all)
CYP1B1, TNFAIP2, ANKRD36C, RIMS1, SCGB3A1, SCGB1A1, NEAT1, STATH, ALDH1A3
Ionocytes
AHNAK, SYNE2, BRWD1, CENPC, LAMA4, NKTR, NUMA1, XIST, PTPN21, HIPK2, RARRES2, AKAP13, MTSS1, LUC7L3,
GOLM1, AKAP9, VPS13C, CHD2, ATP2A3, MUC20-OT1, GNAS, MUC16, DST, TCERG1, ABR, TCF25, CHD6, KMT2C, SCIN,
DGKI, PATJ, SRRM2, GOLGB1, PSD3, MUM1, TFCP2L1, CHD4, TACC2, SLC29A1, DCDC2, RBM5, OBSCN, RABL6
Macrophages (all)
JAML, FAM118A, CLEC10A, LRRK2, ATG16L2, CSF3R, CAPS, AOAH
Secretory Cells (all)
ABCA13, UPK1B, ALDH1A3, ALDH3A1, RIMS1, TNFAIP2, TPT1, MUC4, SPRR3
Squamous Cells (all)
SLC5A3, UCA1, ATP1B3, VEGFA, SLC38A2, TIMP3, GNE, GLS, RNF19A, AKR1B1, ARHGAP5, AKAP9, CRIM1, AQP3,
S100A4, BAMBI, PRRG4, VWF, GDE1, TRIM2, PKM, PRSS3, APP, PARD6B, NIPAL3, GSPT1, RPTN, WDR26, CEACAM5,
ABCA13, CNN3, MIR22HG, GAN, LDHA, EHD1, IFRD1, B3GALT5, PHACTR2, FAM107B, RANBP9, DDX3X, TOB1, UBL3,
USP38, ATP1B1, DAP, MTPN, SPTAN1, FAM102A, PHACTR4, ARHGEF12, MAPKAPK2, PPP1R15B, TP53INP1, YOD1,
CCNG2, ANKLE2, ZBTB38, AHNAK, RALA, KDM5B, DHX32, CPEB4, LRRFIP1, H2AFZ, FAM84B, HECA
T Cells (all)
SYNE1, HSP90AA1, TXNIP, HSPA1A, AHNAK, ITGB2, HERC1, HSPA1B, MACF1, SLA, ITGAL, DNAJB1, SUN2, ARHGEF1,
ANXA1, HSPH1, MYCBP2, DYNC1H1, HUWE1, CYFIP2, SNRNP200, RNF213, NLRC5, SRRM2, NACA, SPTAN1, DDX3X,
SPN, HLA-E, FLNA
AZGP1 high Goblet Cells
TRIM31, CYP1B1
BEST4 high Cilia high Ciliated Cells
TBC1D8, ALDH3A1, ALDH1A1, GSTA2, GSTA1, PROM1, PRDX1, PLAC8, TNFAIP2, TXN, ADH7, CP, ABCA13, PIGR, ETV6,
TSPAN19, CHST9, DPY30, ADGRF1
BPIFA1 high Secretory Cells
SLPI, TPT1, GPRC5C, ABCA13, ARG2, WFDC2, SAT1, LCN2, PLEKHS1, SLC25A3, GNAS
Cilia high Ciliated Cells
TBC1D8, GSTA2, ABCA13, SFPQ, DNAH5
Early Response FOXJ1 high Ciliated Cells
RBM3, DDX3Y, GCLC, PROM1, SRPX2, IL33, TNFAIP2, CD200R1L, GSTA2, TBC1D8, ADH1C, ANXA3, METTL27, NOL6,
FAM129A, KCNE3, CROCC2, FABP6, APOD, NEAT1
FOXJ1 high Ciliated Cells
TBC1D8, RBM3, TNFAIP2, PROM1, GSTA2, CP, SAT1, TMC5, PIGR, IGFBP7, SAA2, LCN2, SEC14L3
MUC5AC high Goblet Cells
ALDH3A1, TNFAIP2, CYP1B1, SCGB1A1, LGALS3, TPT1, CP, ETV6, CD63, CEACAM5, BPIFA1
Interferon Responsive Ciliated Cells
SCGB1A1, TBC1D8
ITGAX high Macrophages
JAML, NLRP1, LRRK2, JAK2, FGD2, CCDC88B, AHNAK, RNF213, FAM118A, HLA-DPB1, MYCBP2, ATG16L2, SETX, LYST,
PLCB2, ITGA4, CPSF1, AKAP13, HLA-DPA1, CAPS, CLEC10A, MS4A6A, HLA-DQA1, ARHGAP4, TNK2, AOAH, CSF3R,
CD74, FRY, TRIM44, UTRN, SAMHD1, NOP53, HLA-DRB1, HLA-DMB, HERC1, USP15, ADGRE5, SRRM2
SCGB1A1 high Goblet Cells
RIMS1
SPRR2D high Squamous Cells
PRSS3, TPRG1, SOX15, RNF222
CCL5 high Squamous Cells
TBC1D8
CD8 T Cells
HSP90AA1, HSPA1A, TXNIP, AHNAK, SYNE1, HSPA1B, DNAJB1, HLA-B, ITGB2, HLA-E, DDX3X, ACTG1, SUN2, ITGAL,
ARHGEF1, MALAT1, HSPA8, RNF213, TPT1, HUWE1, RBL2, SPOCK2, HLA-A, MACF1, HLA-C, SLA, UBC, BTG1, FLNA,
HERC1, ACAP1, DDX17, FYB1, ARHGAP30, TCF25, AKNA, SPTAN1, SNRNP200, GLG1, KMT2A, NOP53, DUSP1,
DYNC1H1, RBM39, SMG1, NACA, PRRC2C, ATM, IGF2R, MSN, SRRM2, SORL1, RBM5, ITGA4, HLA-F, SPN, B2M, TMC6,
PTPRC, CYFIP2, NLRC5, DDX5, SYNE2, ACTB, MYO9B, MYCBP2, EEF2, JAML, TLN1, LCP2, GNAS, ZC3HAV1, NFATC3,
AKAP13, TNF, HSP90AB1, ARL4C, DIAPH1, MYH9, TMBIM6, CHD3, TMC8, TAGAP, KIAA1551, NEAT1, CFLAR
VEGFA high Squamous Cells
ARHGAP5, APP, GLS, ATP1B1, AKAP9, TIMP3, RNF19A

TABLE 4
Differentially Expressed Genes Between Cell Types from Control WHO 1-5 (Mild/Moderate) vs. COVID-19 WHO 6-8
(Severe). Related to FIG. 3. Results from the comparison of cells from each cell type between COVID-19 WHO
1-5 (mild/moderate) vs. COVID-19 WHO 6-8 (severe) individuals. (Implemented using the FindAllMarkers function
in Seurat, test.use = “negbinom”; Genes included with adjusted pvalue < 0.001, logFC >
0.25; Cell types without sufficient cells to test or fewer than 5 significant genes meeting the cutoffs are not listed).

Table 4A. Expressed in COVID-19 WHO 6-8 (severe) individuals
Basal Cells
COL7A1, KRT15, TNC, TP63, TSHZ2, DST, SERPINF1, DYNC1H1, AKAP9, NEBL
Ciliated Cells (all)
AHNAK2, XIST, CFAP53, CKB, FKBP5, P4HA2, LDLRAD1, RSPH1, HSPA8, NELL2, ENKUR, IGFBP5, FAM216B
Developing Ciliated Cells
SAA1, KRT7, NFKBIA, TM4SF1, RBM3, KRT19, BPIFA1, KRT23, DUSP1, MACC1, RND3, GDF15, SAA2, HSPA1A,
C15orf48, S100A6, H1F0, CD63, CXCL8, JUN, NCOA7, MUC1, TACSTD2, FOS, DNAJB1, S100A2, PPP1R15A, TMSB4X,
TMSB10, ATF3, CAPN2, UBC, ELF3
Goblet Cells (all)
S100A9, S100A8, TSPAN1, LCN2, SERPINB3, KRT19, AGR2, PI3, PSCA, S100P, CYP4B1, ALDH3A1, XBP1, SERF2, AQP5,
CTSD, WFDC2, DHCR24, GLUL, VAMP8, GSTP1, SCD, CEACAM6, P4HB, SAA1, DYNLL1, FUT2, GPX2, PKM, LDHA, TSPO,
MYL6, OAZ1, ENO1, FXYD3, CAPNS1, CSTB, KRT7, TACSTD2, PRDX1, GAPDH, CALR, HDLBP, HSP90B1, CAPN2, MSLN,
CSTA, UQCRQ, TALDO1, MUC1, KRT18, ACTB, HSPA5, AZGP1, ACTG1, CD24, TKT, FAM3D, FDFT1, PGK1, COX5B,
SLC44A4, SYNGR2, HSPA8, AKR1A1, TMSB10, ASRGL1, ARF1, ST6GAL1, IFITM2, CANX, TUBB, ELF3, KCNE3, SELENBP1,
NQO1, TSTA3, ATP1B1, HSP90AB1, LGALS3, RDH10, PRDX5, ATP5F1B, LDLR, GUK1, COX7A2, YWHAB, S100A16,
DAD1, ELOB, BLVRB, ASCC2, TSPAN13, C1orf116, RARRES1, GOLM1, GSTK1, KRT8, CD36, SORD, ALPL, A4GALT, DCXR,
UQCRH, NANS, CCDC69, TJP3, SFN, COX7B, UPF1, EDF1, ANXA2, CAP1, PSMA7, ARHGDIB, COX6B1, GRN, PDIA6,
DSTN, CEACAM5, SMIM14, RARRES3, ANAPC11, CD151, HLA-A, S100A6, DBI, FDPS, PDIA4, CDK2AP2, ATP5MG, F3,
EIF3K, CREB3L1, PCBP1, NDUFA4, STARD10, CLTB, LMAN2, VCP, ENSA, FBXW5, MVP, PGD, MLEC, TPI1, LYPD2, TUFM,
GALNT7, GNG5, PTTG1IP, HLA-B, C3, NDUFA6, LRP10, CALM1, HLA-DRB5, ECH1, RPN2, MISP, ARPC2, NAPRT, CLTC,
SLC2A1, DUOX2, SLC6A14, TMBIM6, UBA52, VWA1, BRK1, PSAP, MAL2, ERP29, COX7C, TXN, CIB1, ACSL3, SPDEF,
NDUFS7, ST14, BCAS1, TRIP6, COPZ1, TMED9, CST3, PPDPF, S100A14, CD55, CFL1, DEGS2, B4GALT5, DUOXA2,
ATP1A1, VSIG2, SLC9A3R1, PSMB4, CTNNA1, S100A10, OST4, TMPRSS4, GNAS, MDH2, SREBF1, UBL5, EID1, HSPB1,
RAB2A, NDUFS5, PSMB6, ATP6V0E1, HNRNPC, NDUFB2, FBP1, HNRNPA2B1, PPCS, SSR4, UQCR10, PDIA3, CLDN4,
FKBP2, ASAH1, GALE, ECHS1, PABPC1, SH3BGRL3, BACE2, COX8A, CYP2F1, SRI, SEC31A, ATP5F1A, IQGAP1, APRT,
TXNDC17, UQCRC1, EIF3I, UQCR11, SDC1, SDF4, COX4I1, CAPN1, QSOX1, PPIB, RAB10, FCGBP, RAB25, SOD1, GCNT3,
GNE, FTL, CALM2, CHMP2A, ATP5MC3, PSMB8, YBX1, PHPT1, MRFAP1, SCAMP2, CCT3, PROM2, ACTN4, C19orf53,
COX5A, PSMD8, HLA-DRB1, ATP6V0B, C1orf122, POLR2L, NR2F6, FTH1, MUC4, CLTA, ANXA11, GMDS, SLC25A5,
TPM4, TMED3, VILL, COPB2, TUBB4B, SPRR3, SMDT1, TM9SF3, SMIM22, SERINC2, SERP1, KRT10, CHP1, MIEN1,
S100A11, MSMO1, HINT1, JPT1, CCT5, FAM120A, PPA1, ATP6V1F, SEL1L3, OS9, MYL12A, CAPN5, TOP1, NDUFAB1,
NDUFB10, EIF4G2, ALDH3A2, SSR1, FAU, RNH1, UBC, ARL1, ATP6V1G1, NEDD8, MTPN, TAGLN2, ASS1, RASSF7,
PSMB5, MBOAT2, COPS9, ARF4, KRT4, FUT6, RHOA, YWHAZ, RALBP1, PLPP2, FASN, FLII, ALDH1A1, GDI2, CSDE1,
ATP5MC2, FAM83A, JUP, PTPRF, LITAF, ATP5MD, SEC61A1, RNF10, TUBA1C, NDUFB7, FLNB, HYOU1, TXN2, PSME2,
RACK1, NME1, RAC1, PSMD2, FKBP4, ACTR3, PPP1CA, SH2D4A, UFC1, MVD, ICMT, PHB, ECI1, ARRB2, CTSH, CALU,
PSMB3, TMEM219, RHOC, SEC13, ATP5ME, CXCL17, DNAJB2, ASPH, SEC61B, PRRC2B, VMO1, STEAP4, CD59, MGST2,
PSMC5, SAP18, LAPTM4A, CNDP2, SPINK5, CTSB, PARK7, TMEM54, MLPH, ATP5PB, SLC16A3, TMC5, FAM96B,
KPNB1, TPM3, GNAI1, EEF2, CYC1, INSIG1, H2AFZ, COA3, COPS6, TCIRG1, FBLN1, UGP2, B2M, RAB11A, HSBP1, ACO2,
MCL1, SSNA1, TMED10, GOLGA2, BCAP31, MUC20, EIF1, VAMP5, UQCC2, VPS28, DAP, GNB1, SLC15A2, CREB3L2,
TXNL4A, NAXE, WDR83OS, CMAS, ERBB2, REX1BD, PRKAR1A, SCP2, ATP5F1E, EBP, TRIR, TMEM59, GLRX, AKR1B10,
ANXA4, PCYOX1, MEA1, WDR1, CALM3, TM9SF2, TSPAN3, RAB7A, CCT7, PLXNB2, PLAC8, RETSAT, XRCC5, UQCRB,
TMEM165, COPA, COX6C, SAA2, EIF6, GPI, UBA1, SEC11C, LSM4, PTP4A2, PPL, CHCHD10, SRD5A3, H2AFY, AHCY,
GPAA1, COX6A1, PSME1, EPHA2, AP2M1, CUTA, NDUFS6, EFHD2, CD74, SNU13, NDUFB4, NUCB1, HNRNPM,
HIGD2A, ARPC5, MANF, ZNF185, LASP1, SLC44A2, EPAS1, STS, CKAP4, CFB, PDZK1IP1, JTB, ITM2B, XRCC6, GPX4,
ADGRF1, BMP3, ID1, IFITM3, PPM1G, AQP3, CAPZB, PERP, SRPRB, EPS8L1, KDELR2, VDAC1, LRRC59, TMEM141,
PRSS8, RAB37, NAA38, ATP5PF, SLC25A3, PFKP, B3GNT3, BAG6, ME1, SPTLC2, LAMTOR5, TMEM258, TAF10, CNN2,
CCT2, CRACR2B, VWA5A, SSU72, MAGED1, AES, PSMD1, TIMM13, TUBA1B, ENC1, HADHA, CERS6, SMAGP, PLEKHS1,
GADD45GIP1, KLF5, UQCRC2, CYP4X1, DNAJC3, LMNA, CDC42, CXXC5, ATP5PO, SERBP1, GFPT1, SNRPD2, TSC22D1,
RHBDL2, GLTP, VSIR, AP2S1, MPZL2, SLPI, PIK3R3, ACTR2, RAN, ALDH3B2, PFDN5, CHCHD2, SARAF, ABLIM1, CD81,
UBE2L3, AK2, BAG1, PPP1CC, PDAP1, COMT, MDH1, MYH9, TCTA, DYNLT1, SPINT2, RRBP1, APEH, GHITM, DHRS3,
SLC16A7, DHCR7, EIF4G1, SNX17, ARPC3, MYL12B, ATPSPD, SOX4, PPP1R7, DSG2, EI24, H3F3B, MARCKS, DNPH1,
BDH1, TST, NAGK, PNKD, GSPT1, TRIOBP, NDUFA1, DYNLL2, RNF7, KIFSB, FEZF1-AS1, NDUFB9, UBXN4, SREBF2,
CSRP1, PLIN3, HLA-DRA, TMEM208, HSP90AA1, TM7SF2, GNA15, PUF60, KDELR1, ST6GALNAC1, RNPEPL1, C6orf132,
PSMB1, AUP1, GNG12, ACSL1, JPT2, SLC25A11, SND1, CRK, CRELD2, ZNHIT1, ERG28, SRP14, CTTN, HSD17B10, F11R,
DTX2, CYB5R3, NHP2, TMEM147, SLC1A5, REXO2, SF3B2, C12orf57, PPP1R11, RAB1A, ATP5MPL, PTAFR, SELENOH,
EEF1D, ATP2A2, PDXK, ILVBL, HSPA1A, TMEM125, TRIM29, PRSS23, ACAA1, CHMP3, STT3B, OAS1, ERLIN1, CD9,
RAB1B, PSENEN, FUT3, SLC40A1, PGLS, NCOA4, SCEL, NDUFA2, LPCAT3, SELENOW, PSMC3, NDUFB3, LLGL2,
SLC25A39, RBM47, TRPM4, SCNN1A, KRT24, GDPD3, SRSF3, DNAJA4, AP3D1, ALCAM, LGALS8, CBR1, ADI1, ARL6IP4,
SF3B5, SLC39A7, TMED2, SEC62, HACD3, DBNL, RDH11, GM2A, CCPG1, SLC16A9, POLR2E, GRHPR, BTF3, TMC4,
DHRS9, ARPC1B, YIF1A, TAX1BP1, C1orf43, NDUFS2, BCL2L15, STIP1, ADH1C, CNPY3, PQBP1, PDCD6IP, ACACA,
MTURN, MUC16, SYPL1, PAM, COX14, B4GALT1, BRI3, ERMP1, FARSA, HDGF, CD46, CAPRIN1, GDF15, ATP6V1D,
PYGB, PDCD6, PRELID1, KIF1C, TTC1, TMCO4, PHB2, PSMD13, MPC2, HAX1, GNS, ARHGAP1, SPCS3, HNRNPF, NIPAL3,
ANAPC16, C19orf70, STUB1, LRG1, UBE2D3, EFEMP1, GRB2, NOP10, CISD1, MYDGF, TRAM1, EMC4, ROMO1, CCNI,
IDI1, SRPRA, PTMA, SLC25A1, ANXA5, AURKAIP1, SHISA5, GSR, RGL2, POLR2F, SUMF2, AHSA1, AC104126.1, RNPEP,
CLDN7, EMP2, IRAK1, CD164, NDUFA13, ADIPOR1, HTATIP2, DTX4, ADGRG1, TERF2IP, LAPTM4B, PTOV1, MCRIP2,
FAM107B, GMPPB, PYGL, SLC35C1, CANT1, FIS1, HSPA4, TCP1, 2-Sep, DNAJC8, EIF4G3, WASL, LRP11, NDUFB6,
EFCAB14, CAPN13, IRAK3, FAM173A, PTGES, PTBP3, PON2, NFE2L1, NAP1L4, G6PD, TPT1, NIPSNAP1, NDFIP1, PQLC1,
GNB2, ALG3, MORF4L2, FLOT1, VGLL4, SET, IMP3, MAOA, RBX1, RTN4, DDRGK1, CENPX, HLA-DPA1, MTCH1, GIPC1,
EIF4EBP2, TAF7, PSMC2, RSRP1, TMEM205, RNF181, ACLY, PMVK, VCL, GOLPH3, RAB14, HLA-E, STX10, SDR16C5,
CDH1, UXT, ADAM9, PSMB7, KYNU, TFG, CAPZA1, TM7SF3, MMAB, TXNIP, NDUFA12, RND3, DNAJB1, MON1B, ILF2,
EIF3H, CCT4, SNRPC, SOX2, BCR, EPRS, DUSP1, BSG, NACA, LAMTOR4, CYB561, SSR2, PREB, HEBP2, SERPINF1, ARCN1,
TRMT112, SERINC3, NDRG2, PLPBP, S100A4, MPZL1, PSMA3, LRPAP1, CYCS, MTCH2, GOT2, IL13RA1, GORASP2,
NPDC1, ALKBH7, TBC1D9B, SCPEP1, FAM129A, TLN1, HK1, RSL1D1, SPSB2, PFN1, STOM, NDUFB5, ATP6AP1, PSMC4,
ARHGDIA, HMOX2, SNRPD3, PLEKHJ1, CPD, CCT6A, EIF3L, CYB5A, CCT8, DNAJC15, COPG1, PA2G4, CMPK1, TIMM17B,
FKBP3, ACAT2, OTUB1, GABARAPL2, TRNP1, FAM129B, PRKCSH, APLP2, WBP2, ZG16B, FHL2, RPN1, LSM7, CSNK1A1,
GPS1, CXCL16, TIMM8B, YIPF3, MAPK13, MESP1, PARP1, TMEM50B, ACTR1A, HLA-F, TAPBP, SELENOF, JUND,
AP1M2, NDUFB11, SSR3, HNRNPU, NDUFV2, HLA-G, STT3A, MTIF3, TGOLN2, DMAC1, MID1IP1, ARSD, TNFAIP3,
CYFIP1, CD82, DDB1, PRDX3, STOML2, MYH14, ERH, KLF6, ARPC4, CDC37, SQSTM1, POLDIP2, ARF6, UBB, TXNL1,
COMTD1, LMO4, SP1, LRRC8A, GTF3A, CTSA, NDUFA8, DIAPH1, TP53111, DDOST, HM13, SEC63, USF2, DYNC1H1,
GALNT1, TMEM167A, ATP6AP2, TRIM16, SUN2, KIAA0513, DCAF7, DHX29, NIPAL2, MAGT1, BRD2, STAT3, NAV1,
AVPI1, CHCHD1, GGT6, YWHAG, FAT1, AHCYL1, ATXN7L3B, STK38, PSMD14, GTF2A2, CDCP1, MTDH, KTN1, C6orf106,
ATP5IF1, IPO7, YY1, SMARCC1, RSU1, PSMD4, SYT8, PCDH1, GSTA1, SUCLG1, SLC39A11, ESRP2, TNFRSF1A, ATP10B,
POLR21, ELOF1, COPS7A, ZMAT2, TNFRSF21, EIF4A2, ALDH1L1, SDHB, PSMA4, HLA-C, RAB8A, NRDC, ATP5F1C, SAT1,
ERLEC1, PDHB, PSMD11, NDUFB1, UBE2H, YBX3, PTGFRN, SGPL1, GLUD1, SPCS1, ENPP4, NENF, YME1L1, UTP14C,
AHR, HSPA9, TMBIM4, MYO1C, NAGA, TMEM33, EMC3, NEDD4L, SLC52A2, SERTAD1, EZR, ACOT13, ARFIP1, HADHB,
CREB3, TMEM123, FOS, PRDX2, TOX4, VAPA, WASF2, RDX, GPRC5A, ZBTB38, LGALS3BP, BLCAP, EPS8L2, METRNL,
ALDH9A1, C6orf62, CBX6, DDX49, SRP72, PAPOLA, ECHDC1, LDHB, FAM114A1, GLG1, YWHAE, CCNO, PTGR1,
EBNA1BP2, ORMDL2, NAA50, GPRC5C, BUD31, NDUFC1, QARS, POLR2K, MMADHC, SYTL1, MECOM, NDUFS1,
NAA20, HNRNPAB, RNF20, SF3B3, LAMTOR1, ATP8B1, CCDC80, FUCA1, PYURF, MGST1, NCCRP1, FOXA1, KIAA0100,
NUDC, MFSD1, RABAC1, SF3B6, EPHX1, HSPE1, PPP1R15A, FAM83D, GALNT3, PDLIM1, TSPAN6, TIMM23, PPP1CB,
ABHD2, RAB3D, TTC3, ATP6V1A, RNF187, CDKN1A, SPAG7, EPB41L1, SLC38A10, GAA, DGCR2, HDAC1, LARS, TSR3,
CISD3, LAMP1, PPP2CA, KPNA6, PDHA1, KRT6A, RALY, SH3PXD2A, SAR1B, DUSP6, PTPRU, PSMA1, MLF2, IFI16,
PLEKHB2, ATP5MC1, GOLPH3L, NSFL1C, PTGES3, ITGB4, ZC3H14, CLDN1, WNT7B, FAM98A, SCAP, ATXN10, HS3ST1,
UBQLN1, TSTD1, LSM3, DUT, RBCK1, VTI1B, FYCO1, CAST, PIGT, SURF4, VPS25, SYAP1, ADD3, SARS, GDE1, OCIAD1,
SCARB2, PARM1, COPB1, EPS8, PSMB2, UGCG, TUBGCP2, EYA2, EMC10, C4orf3, RAD21, ACOX1, PITX1, CD63, MZT2B,
MET, SSB, PRR15, IDH2, ATRAID, JUN, DYNLRB1, KARS, DDR1, DAP3, SLIRP, HMGCS1, MOB1A, ADIPOR2, EXPH5,
LAMB2, CBX5, UBE2K, POMP, DENR, CLSTN1, CDC42EP3, RAD23A, MATR3, NME3, SLC3A2, GPD1L, UBXN1, SEC24C,
AC025154.2, PLPP5, MAPRE1, CEBPD, TBL1XR1, RABL6, LINC01133, RTN3, AZIN1, TIMMDC1, LYPD6B, CLCN3, ERGIC3,
AGRN, RAB5B, G3BP2, SPINT1, SNX3, S100A13, GCHFR, AGPAT3, MORF4L1, TSPYL1, ESRP1, NARS, MPC1, TUBB2A,
PPP2R2A, CTSS, SPTBN1, CHL1, IER2, SDF2L1, DAG1, SEC11A, SPG21, RTF2, TMF1, ESRRA, EEF1B2, TCEA3, CASP4,
EIF3G, NCL, SKP1, MYO1B, CLIC1, SYNCRIP, EIF4H, AGL, EDEM3, SEC24D, DFFA, NONO, HDAC2, ANTXR1, ATP6V1E1,
KIAA1522, NSF, CYBA, ATF4, SIVA1, HP1BP3, PJA2, NFKBIA, ZBTB7C, ARPC5L, CTR9, PDLIM5, SLC4A11, HINT2,
ACADVL, LAD1, ACBD3, 11-Sep, LIMK2, DDX6, TAP1, CLIC6, UBE2J1, TOMM7, GNAQ, CCND1, ATP1B3, ANP32A, IDH1,
FAM84A, SLC12A2, FIBP, ELOVL5, EIF2AK1, MYO5B, MKRN1, GBP3, POF1B, MKNK2, OCIAD2, PRKAA2, KRT13, PSMA5,
PAPSS1, PSMB9, IKZF2, HMGN1, FAM32A, DNAJA1, EIF4B, CRYM, PIGR, SCRIB, CHST6, STK24, GALNT5, DNTTIP2,
MXD4, PGRMC1, FAM120AOS, IFI27, PEA15, ARL6IP5, C19orf33, TMCO1, OAT, KLF3, RER1, NT5C2, LGI1, SELENOS,
USP22, MAPK1, SMC1A, EIF3A, FNDC3B, AKR1C1, GTF2F1, NDUFS3, XIAP, KIAA1191, UBE4A, DAZAP2, PRMT2, EIF5A,
TMBIM1, HUWE1, NBR1, G3BP1, PRKAR2B, EIF3D, EIF5B, PRRC1, ZDHHC3, CHTOP, ARFGAP3, AHNAK, RXRA, HMGN3,
SEC16A, OSBP, NUCKS1, CTBP2, ATP9A, PILRB, SELENOK, CLINT1, VMP1, GSS, UBE2L6, TMEM9B, CNOT1, HSPA1B,
SYNJ2BP, XPO1, CTCF, TMEM30A, TP5313, NEBL, 7-Sep, CAPN8, PRPF38B, ADD1, CAB39, UNC13B, TMEM50A, JAK1,
CTDSPL, PRKAR2A, PADI1, KHDRBS1, EIF5, VPS35, KCNK6, GOLGA3, RAB5A, TES, IRF2BP2, PTBP1, SORT1, GPT2, IK,
CDC42BPA, CNBP, MAPKAP1, PAFAH1B1, LMAN1, ERBB3, GTF21, MUC21, ZFR, RMDN3, GABRP, MAN1A2, DCAF5,
SERPINB1, ALAS1, PRDX6, CCDC186, PEBP1, SRSF7, TFCP2L1, TMEM87A, H6PD, MAVS, UAP1, RCC2, HTATSF1,
CTNNB1, CHD3, TTC9, RHOV, NOLC1, DNAJC10, CFH, ALDH3B1, SAR1A, CYR61, SERINC1, NCOR1, STARD7, ANP32B,
REEP5, FGFBP1, TRIM26, ALDH2, WNK1, HES1, C15orf48, ANXA1, ECPAS, LARP1, SDC4, RALGAPA2, ELL2, SSRP1,
DHX9, BTG1, ANKRD17, CTSC, LONP2, SOD2, NORAD, GSN, HSPD1, CHMP1B, BAG3, TXNRD1, NDUFV1, KIF13B,
ZNF106, TMSB4X, ITPKC, SMARCA4, CORO2A, ETF1, BAZ1B, ZFP36, SASH1, LIMA1, MAFF, LAMB3, SH3GLB1, NFE2L2,
PLEC, PAQR4, MIA3, TRAK1, MAGED2, HNRNPUL1, BBX, THRAP3, TRIP11, HERPUD1, CAMK1D, SMARCA2, CHMP4B,
SCAF11, ZKSCAN1, HNRNPK, LY6E, HNRNPR, SLC4A4, TRIM56, RAB11FIP1, EHF, TMEM160, UPK1B, CA12, FOSB, DSP,
ADIRF, CRYBG1, UBR4, ATF3, RAI14, ADH7, RNASE1, MACC1, TC2N, JUNB, SARSCoV2-NegStrand, LYN, CTNND1,
HSPH1, MUC5AC, DDX17, PTPN13, IQGAP2, BHLHE40, GADD45B, ABCA13
Secretory Cells (all)
SAA1, CXCL8, CXCL3, S100A8, CXCL2
Squamous Cells (all)
SAA1, KRT16, SERPINB3, PDZK1IP1, GLUL, AGR2, PLAT, HES2, AHNAK2, DSG2, KRT6C, SAA2, S100A8, S100A9,
SERPINB4, GSTP1, FTH1, FCGBP, BEST1, TXNIP, SPRR1B, TRIOBP, RHOC, TSPAN1, HSP90B1, SERF2, KATNBL1, CD59,
SARSCoV2-NegStrand, C3, CYP4B1, CD55, LCN2, SYNGR2, MSLN, ATP5F1E, LRG1, NTN4, NAPRT, SARSCoV2-ORF10,
ELOB, FTL, SERINC2, CANX, SARSCoV2-N, SARSCoV2-3prime, CALR, MUC16, NEAT1, TGM2, S100A12, CSTB, CCDC80,
SARSCoV2-S, UQCRQ, SPRR2D, RSPH1, CXCL17, DHCR24, CD24, CEBPD, S100A7, KRT6A, HDLBP, C15orf48, KRT17,
LAMC2, ERO1A, KRT19, POR, YWHAE, SERPINB1, CALM2, PRDX5, GRN, MUC1, CARHSP1, CD46, PTTG1IP, UACA,
CEACAM7, KLK8, TMEM160, RAC1, NABP1, TMSB10, SDR16C5
AZGP1 high Goblet Cells
S100A9, CYP4B1, S100A8, PSCA, RARRES1, XBP1, KRT19, SERPINB3, TSPAN1, C3, AGR2, LCN2, DHCR24, SERF2,
PLEKHS1, FUT2, AQP5, ASRGL1, HDLBP, SELENBP1, AZGP1, SLC44A4, FAM129A, S100P, GUK1, CD36, IFITM2, SCD,
VAMP8, P4HB, MLEC, HSPA8, SAA1, CCDC69, TUBB, SORD, KRT18, UQCRQ, GLUL, HLA-DRB1, LDHA, MSLN, CAPN13,
OAZ1, C1orf116, EIF3K, UPF1, HSP90AB1, EIF3A, AKR1A1, GALNT7, EPAS1, ACTB, TJP3, PI3, ENO1, CAPNS1, PRRC2B
BEST4 high Cilia high Ciliated Cells
BTNL9, GRAMD2A, CFAP70, TOGARAM2, WDR6
Early Responsive FOXJ1 high Ciliated Cells
AHNAK2, FKBP5, TSC22D3, TFCP2L1, SCO2, SPON2, RYR3, CWH43, HOMER2
Early Response Secretory Cells
FKBP5, CYP2A6, HSD11B2, ARRDC2, CYP4B1, FAM83D, ALOX15B, PDK4, ZBTB16, HCAR2, TSC22D3, SCNN1G,
PPARGC1A, GPX2, CYR61, HCAR3, CFD, SCNN1B, GLUL, DEPTOR, TP5313, TFCP2L1, XBP1, DHCR24, HDAC5, LRRC26,
CELF2, STEAP3, MSMO1, SLC26A2, DUSP1, TCIM, ATP1A1, IDI1, PTGS2, PDE4DIP, FLT1, LDLR, DUSP2, DHCR7,
HMGCS1, DUSP6, SORD, SERPINF1, MAFF, SLC6A8, FDFT1, SQLE, NDFIP1, CYP2A13, TENT5C, KRT24, NR4A1
FOXJ1 high Ciliated Cells
AHNAK2, LDLRAD1, RSPH1, FKBP5, CKB, HSP90AA1, CYP4B1, KRT18, HSPA8, MAPK8IP1, APBB1, DRC1, BAIAP3,
MAP1A, ENKUR, NELL2, FAM216B, RIBC1, NFE2L1, RYR3, PTPRT, P4HA2, OSBPL6, MGLL, SOD1, NFX1, HSP90AB1,
NLRP1, CARS, SMC1A, TRAF3IP1, COBL, CCDC113, PEX6, SAMHD1, PBXIP1, ERP29, ACACA, AGR2, ANXA4, MAP6,
CYB5A, SAMD15, PLEKHB1, IGFBP5, IL5RA, APOD, STMND1, CFAP45, CFAP100, CDKN1A, ZNF106, SLC44A4, MYH10,
UCHL1, LRRC6, PPIL6, DTHD1, DNALI1, FTH1, PPP1R16A, ARL3, CFAP53, MPC2, OAZ1, HEATR5B, DNAJA1, HNRNPC,
HSPA4, ERBB4, FHAD1, CCDC190, PDCD6IP, C1orf87, C11orf88
MUC5AC high Goblet Cells
S100A9
Interferon Responsive Ciliated Cells
BTNL9, S100A9, BDH1, CFAP53, CACYBP, METRN, HSP90AA1, S100A8, LINC01436, LYPD2, HSPB1, SRGAP3-AS2, SAA1,
LCN2, HSPA8
SCGB1A1 high Goblet Cells
GPX2, SERPINB3
SERPINB11 high Secretory Cells
SAA1, COX6A1, BPIFA1, ACTB, FTH1, CXCL8, TXN, TMSB4X, HSPB1, FTL, S100A11, AQP5, PTMA, MIF, HES1, GLUL,
CTSD, COX7A2, SLC25A5, UQCR10, YBX1, KRT7, UQCR11, UQCRQ, NFKBIA, NPM1, KRT24, PSAP, PRDX1, PFN1, JUN,
H3F3B, DNAJB1, GSTP1, ASS1, TAGLN2, TPI1, ANXA1, JUNB, CYR61, ELOB, KRT8, HEBP2, TKT, COX7B, CLDN7, SERF2,
GSR, ZFP36, CDC42, H1F0, F3, PPP1R15A, APRT, ENO1, PPP1CB, PLK2, CAP1, HNRNPK, GAPDH, CLIC1, DUSP1, KLF6,
MORF4L1, TPM4, NCOA7, PPP1CA, GDI2, RAC1, IER2, RBM3, SLIRP, JUND, PTTG1IP, ELF3, PABPC1, ATP5MF, NDUFA4,
ATP5MC1, EIF1, HSPA1A, PPIB, HINT1, BHLHE40, SRSF3, S100A14, CBX3, YWHAE, COX6B1, PSMB6, UBB, TMED2,
ATP5PO, DUSP5, C1orf43, ATP5ME, CSTA, HNRNPA1, COX5B, ARHGDIB, CD55, EEF2, PKM, SPTSSA, SET, H2AFZ,
VAMP8, PPDPF, CD9, KRT23, DHCR24, UBC, SOCS3, PSMA7, SAA2, AHR, PRSS23, TMA7, SERPINB4, S100A9, C19orf33,
NDUFA13, NDUFB2, EEF1A1, SLC38A2, RND3, RAB10, MYL6, DSG2, NDUFA6
CCL5 high Squamous Cells
GLUL, SERPINB3, KRT19, S100A9, S100P, FTH1, SLPI, KRT7, KRT24, PSCA, GSTP1, WFDC2, LCN2, TSPAN1, CYP4B1,
CAPN2, MUC1, TACSTD2, C15orf48, S100A8, SPRR1B, SAA1, CD55, VMO1, DHCR24, CD59, ANXA2, AHNAK2, VAMP8,
CCDC80, ANXA1, ATP1A1, TXNIP, PRDX5, EZR, CALM1, HSP90AA1, FOS, MUC16, MALAT1, AGR2, KRT6A, C9orf24,
MYL6, RSPH1, DUSP1, SARSCoV2-NegStrand, LGALS3, FAM216B, GDF15, SLC44A4, CIB1, KRT17, CDHR3, ELF3,
ALCAM, DSP, SARSCoV2-ORF10, CKB, MUC4, TMEM59, BPIFB1, CLDN4, SARSCoV2-N, PSAP, CYR61, CAPS, CD24, HLA-
A, SOD1, ATP5IF1, HSPA1A, HLA-B, AHNAK, H3F3B, SARSCoV2-3prime, FTL, CANX, HSPA1B, TPPP3, SERF2, SARSCoV2-
S, NEAT1, TPT1, TPM4, GNAS, KRT23, CTSD, CDKN1A, PPL, CLU, ATP1B1, GPRC5A, TSPAN3, KLF5, TMBIM6, CALM2,
SAT1, B2M, F3,
Table 4B. Expressed in COVID-19 WHO 1-5 (mild/moderate)individuals
Ciliated Cells (all)
TBC1D8, IFI44L, IFITM3, ISG15, PARP14, MX1, IFITM1, IFI44, RNF213, STAT1, RBM3, SLC6A6, IFI6, OAS2, SAT1,
AD000090.1, PROM1, OAS3, TRIM22, IFIT3, STAT2, IFIT1, IFI27, ETV6, RSAD2, TMC5, MX2, CMPK2, DUOX2, XAF1,
HERC6, LAP3, PLSCR1, PIGR, LY6E, TNFAIP2, PARP9, DDX60, ATP12A, RHBDD2, STOM, FUS, SFPQ, GSTA2, OXTR, IRF1,
ISG20, GBP1, OAS1, IFI16, APOL1, DDX3Y, SAMD9, AKR1C2, CBR1, ABCA13, S100A6, SP100, SPG7, COLCA1, TRAF4,
PLAC8, PALLD, WDR90, SLC4A11, CXCL17, SAMD9L, UBE2L6, ODF3B, PSME2, CTSH, LGALS3BP, LYN, TRIM8,
UNC93B1, SLC37A1, APOL6, UBXN11, POMT2, ADAR, IL33, DDX24, TAPBP, TMEM173, TAP1, PRSS23, PGD, EIF2AK2,
PRDX1, ACTG1, ACTB, PLXNB1, ATP1B1, ELF3, VNN3, CYB561A3, GCLC, CRACR2B, AL357093.2, RARRES3, CCDC189,
CTSS, AQP3, MUC1, ALDH3A1, PLXNB2, HLA-C, ITM2B, CTNNAL1, FTO, S100A11, AKR1B10, SDC4, NUCB2, PFKP,
PSMB9, FAM107B, MUC4, ADH7, PLTP, MDK, GAPDH, SPATS2L, TXN, PLEKHS1, ANXA11, B2M, CES1, CTSB, GSN, VIM
Developing Ciliated Cells
ABCA13, HIST1H1C, CYP4B1, CFAP53, KIF21A, DZIP3, TMEM212, CFAP43, TUBA1A, ENKUR, IGFBP5, KTN1, HSP90AA1,
CCDC146, DNAH9, CCDC113, TUBB4B, DNAH5, ARMC3, RSPH4A, TSPAN6, AC007906.2, WDR78, PCM1, DNAH12,
SYNE2, MDH1B, CETN2, SAXO2, ALDH1A1, SAMHD1, HSP90AB1, AKAP9, ZNF106, FMO3, IQCG, BBOF1, NWD1,
DNAH6, HSPH1, SPTLC2, KLHL6, VWA3B, AGR2, LDLRAD1, NUCB2, ARHGAP18, UBXN10, ADH7, CFAP45, SYNE1,
C11orf88, TNFAIP8L1, HNRNPA2B1, OMG, DYNC2H1, RSPH1, CC2D2A, TPPP3, CCDC170, IFI27, SPEF2, FHAD1,
AL357093.2, CFAP157, DYNLRB2, PIGR, UFC1, PIFO, DNAJA4, SNTN, TMEM190, DYNLL1, C20orf85, SPATA18, PTGES3,
MNS1, CDHR3, SCGB2A1, CAPSL, C4orf3, MAP1A, EEF1A1, FAM216B, HSP90B1, CD59, DDX17, AK7, SERF2, HLA-DRA,
ZBBX, SPA17, SARAF, CALM1, MMACHC, IFT57, MUC16, HSPA8, ERICH3, LRP11, LRRIQ1, SOD1, CFAP44, NQO1, IK,
DNAJA1, ARL3, STAT1, IFI6, CAPS, TSPAN1, CD24, CD74, TMBIM6, C9orf24
Goblet Cells (all)
PARP14, SCGB1A1, IFI44, PLEKHH1, IFI44L, TNFAIP2, TMEM213, NEAT1, BPIFA1
Ionocytes
RARRES2, FOS, HLA-C, JUN, GNAS, DNAJB1, SCNN1B, APLP2, ACTG1, UBC, GOLM1, HSPA1B, RBM3, BRD2, MUC20,
AKR1B1, KRT18, DMRT2, SRSF3, RBP1, FBXO32, KRT8, SEMA3C, CBR1, EZR, GADD45B, FTH1, PPP1R12B, CCNI,
AHNAK, HSPA1A, PSAP, TPT1, HLA-E, ID2, NDUFB9, ATF4, BHLHE40, HLA-F, DAP, LAMA4, ITIH5, SCNN1A, MYL6,
PTPN21, PEBP1, EGFR, ECE1, DUSP1, SPTBN1, ATF3, HLA-B, ACTB, PLK2, SLC25A6, TIMP3, PGD, CDV3, DHCR24,
SCNN1G, NACA, LMO7, CD81, STK24, 9-Sep, TFF3, CHD4, TUBB4B, S100A6, CSDE1, EEF2, LGALS3, STAP1, KLF4,
RACK1, FAU, NBL1, KRT7, HERPUD1, SLC3A2, NEDD4L, ATP1A1, NONO, FOSB, DDR1, HLA-A, ENC1, PFN2, MAOA,
IMPA2, SCARB2, SSFA2, TAOK1, FTL, FAM120A, TACSTD2, TBC1D1, COX8A, ARHGAP35, ERLEC1, KIT, OS9, JAK1,
RHOB, ARHGAP18, VPS13C, ZDHHC3, LGR4, COX4I1, KLF6, HIPK2, EEF1D, TC2N, HDGF, EIF1, CD63, IFI27, TMSB10,
ATP1B1, BSND, ENO1, GCLC, NR4A1, SIRT2, ABHD2, FOLR1, CFTR, PKM, CD24, DYNLL2, ZNF217, PIM3, HSP90AB1,
SCAND1, NCL, WDR1, GSTP1, GAPDH, RGS2, FOXI1, STAT1, LAMB2, RHBDD2, FAM102A, NOP53, PACRG, CD74,
CAPNS1, SDC2, AKAP7, CIRBP, GNA11, CAPN2, PCBP1, AP2M1, TMEM9B, PSMB3, ZFAS1, GABARAPL1, UBB, TXNRD1,
TAPBP, IMPAD1, CD9, PPP2R5C, B2M, EIF2AK1, CIB1, ARF1, ATP2A3, CUTA, EIF4G2, DSP, ARPC2, EMC10, DDB1,
HSPA5, ATP5F1B, TFCP2L1, ATP6V0B, MAL, PERP, MAN2A1, PSMB4, KIF1C, MTSS1, MYO1B, ADM, PRDX5, TMEM14C,
AKAP13, COX5B, TMBIM6, KDM5B, DAG1, SH3BP4, PRDX1, NDUFS3, AUTS2, TSPAN6, SF3B2, STK39, HNRNPA2B1,
KLHL24, MYOF, TMEM59, LMO4, PBXIP1, CLCNKB, UBE2D3, EIF5, CSNK1A1, PTTG1IP, C1orf115, ATP13A5, ATP6AP1,
HEPACAM2, GUK1, ADAR, POSTN, P4HB, AES, ASCL3, PPFIA1, REPIN1, DNAJC10, C12orf57, KCNQ1, PTP4A1, H2AFY,
SIN3A, EIF3A, MX1, SF3B1, APOL6, TRIP12, NR1D2, MTDH, ZG16B, POLR2E, RASD1, PTDSS1, ACTN1, ATP6V0A4,
PDIA3, EFHD2, PCBP2, RERE, HYOU1, SARAF, SSR1, SREBF2, EPCAM, ITM2B, ETS2, FIS1, GPRC5B, ACO2, CALM1,
CCT6A, SQSTM1, MTUS1, RNH1, MRFAP1, ATP5IF1, SPOCK1, MYH9, BRI3, ATP2A2, ESRRA, INPPL1, PTP4A2,
AD000090.1, NARF, IFI6, DNAJA2, STARD7, MIR22HG, CANX, KLF10
Macrophages (all)
TMSB10, CLEC10A, CST3, RUBCN, PARP14, ADAM19, JAML, STAT1, MS4A6A, RNF213, ANKRD22, PPA1, CALHM6,
SERPING1, PSMB3, MYD88, CD74, ALDH3A1, CD274, LMNB1, HLA-DRA, HLA-DPA1, GBP4, WARS, LSP1, HLA-B,
ARID5A, SAT1, HLA-A, BRD2, IER5, CXCL9, GBP2
Secretory Cells (all)
PARP14, MX1, STAT1, HERC6, ALDH3A1, TNFSF10, RNF213, ABCA13, CCDC80, AQP3, IFI27, XIST, IFI44L, HLA-E, IFI44,
MDK, PIGR, FAM107B, RBM25, APOL6, ADAR, SP100, SYNE2, ALDH1A1, VPS13C, CPLANE1, APOL1, OPTN, SLK, LMO7,
CTSB, PRRC2C, TPT1, XAF1, HLA-C, TSIX, MUC16, EPSTI1, ADAM28, MTUS1, FER1L6, HSP90AA1, KIF21A, AHNAK,
PSCA, GOLGB1, OAS2, APP, SLC6A6, IFITM1, CEACAM5, WARS, EIF2AK2, LAP3, CALM1, SMCHD1, AKR1C2, HLA-DRB5,
NFAT5, AKAP13, APLP2, RSAD2, NCL, PARP9, ATP13A5, ZNFX1, PLEKHH1, RBM39, IFIT1, PRSS23, CD74, EZR, STAT2,
SAT1, ANXA11, ABLIM1, TXNIP, GOLM1, IFITM3, POR, PTPRZ1, CP, TRIM22, TRIP12, PLXNB2, BST2, DDX5, SLC31A1,
PLSCR1, TAP1, SORL1, VMO1, KIAA1551, DHRS3, F2R, ANK3, UBE2L6, IFIT3, MYOF, PHF20L1, PPFIA1, KIAA0319L,
SRSF5, DDX60, PGD, SLFN5, SAMD9L, OAS3, LIPH, GOLGA4, BAZ2A, OGFRL1, EML4, PTPN13, KIF13B, ARHGAP18,
XRN1, MACF1, ERN2, GAPVD1, TCERG1, FAM3D, DYNC1H1, SRSF11, ATF4, DSP, MUC4, SH3RF1, SAMHD1, ECE1,
PEBP1, CCPG1, TET2, SYTL2, STK24, DDX58, CTSS, ASH1L, ZFAND5, SFPQ, NOP53, PERP, KLF5, EIF4G2, EPAS1,
ARFGEF1, HLA-A, BOD1L1, LMO4, BRD2, TACSTD2, MX2, ISG15, UBB, ADH7, HLA-DQB1, PHACTR2, SAMD9, CHD3, F3,
KIF1C, SREK1, RRBP1, EIF5, ST6GALNAC1, PSMB9, NT5C2, ANKRD12, HSPA5, ATP7B, LRIG3, RNF19A, C6orf132,
LGALS3BP, ETV6, PDXDC1, SECTM1, KIF5B, TAPBP, EHF, RIOK3, ITM2B, CHD4, TRIM33, FNBP4, TGM2, PABPC4, HDGF,
UBC, MTDH, MYLIP, ITPR3, TCF25, SCAF11, SLC28A3, NCOA4, GK5, CHD6, GGT6, HLA-B, FBP1, ITGB8, EIF3A, CTDSPL,
CTNNA1, CRYBG1, TRIM2, LYN, CLINT1, TPR, HECTD1, ZMYM2, TYMP, H2AFY, MACC1, CTNND1, ALOX15, TRIM44,
TGOLN2, USP34, ST8SIA4, ERBB3, OAS1, KMT2C, ATP2C2, CHMP4B, ABCC4, HSP90AB1, PLXNB1, CYLD, UTRN, CD82,
DTX4, IFI6, GSN, SERPING1, S100A4, HLA-DRA, FLNB, CAST, ILF3, C6orf106, CFLAR, DDX24, PABPC1, PLEKHA5,
ZNF207, LIMA1, ALCAM, SF3B1, ARHGAP5, KTN1, EFHD2, PRKAR2B, CRACR2B, TCN2, SETD5, CHD2, SDC1, PPM1L,
SQSTM1, LRP10, ANP32B, SLC25A36, CNDP2, CLDN4, B3GALT5, GALNT5, CD151, RARRES3, DDX3X, BPIFB1, NQO1,
DUOX2, PDLIM1, CCND1, PTPN3, ST14, NFIB, ATP10B, RAB37, VPS13D, FMO3, KIAA1217, SCO2, ARHGAP35, PSME1,
ZNF326, CSDE1, IFI16, CLSTN1, CDK5RAP2, IQSEC1, BCAS1, OGA, SASH1, EEF2, ARHGEF12, TSPAN3, FOXN3, DDX46,
UBR5, AKAP9, CMYA5, DDX17, NACA, UBXN6, FAM129B, ADH1C, TM9SF3, MYH14, ALAS1, CCDC6, WDFY1, SRSF2,
SENP6, C3, PNN, ADGRF1, ACAP2, WAC, ATRX, TRIM26, SFSWAP, ANKLE2, GDE1, PKM, SARAF, RAB11FIP1, HSH2D,
RCC2, LCOR, SNRNP200, AP3D1, MFSD4A, EWSR1, NCK2, LGALS8, RSRC2, TXNRD1, KDM2A, CFH, STARD10, PPP4R1,
SON, DHX32, AKAP8L, TNFAIP2, RABGAP1L, SLTM, MYCBP2, SLC5A3, PTGES, CYP2S1, PNISR, GSTP1, FXYD3, VPS13A,
MYO1B, PLEKHG1, SRRM2, ZDHHC13, HNRNPU, NR2F6, HDLBP, TC2N, LY6E, RACK1, PHF3, SSFA2, GSTK1, PLPP2,
KIAA2026, CD2AP, HNRNPA2B1, HNRNPDL, ANKIB1, HNRNPUL1, PCM1, RERE, PRPF40A, CPEB4, TAF15, CIRBP, ADIRF,
SMARCA2, CD63, NEURL3, GAK, GRHL1, NDRG2, EIF2AK3, ARHGAP32, ZNF292, DUS1L, TMPRSS4, ARGLU1, S100A16,
SPTAN1, MAN1A2, KIAA0513, STIM2, TMEM87A, TNKS2, WNK1, DNAJC2, SMAD3, CCNL2, MARK3, VPS37B,
HERPUD1, SPTBN1, SPG7, MBD2, CSNK1G2, CD59, AHCYL1, LMAN1, CNOT1, ZBTB7A, IGFBP3, LIMK2, ITCH, SUPT6H,
CLIP1, RORA, TRAK1, FAM214A, FCHSD2, PFDN5, SLC38A2, PRDM2, ELF3, CORO2A, RHOB, BAZ1A, EIF2AK1, EPS8L2,
SLC37A1, ANKRD11, MIR22HG, CSNK1D, EEF1D, ZNF644, PTBP1, ZFAS1, PER3, BAZ2B, NCOR1, LRRFIP1, CREBBP,
SETD2, GCLC, NUB1, TMBIM6, CHST9, CCNT2, USP53, WNT7B, PTTG1IP, PSME4, COX4I1, ADD3, N4BP1, CCDC88C,
KIF2A, SELENBP1, CUL4A, SLC25A28, NUMA1, MUC1, MAP3K8, TTC3, USP15, DCAF5, RASSF7, TP53I11, STRBP, EIF3B,
RAB11A, SEC14L2, SRRT, WDR26, ME3, FNDC3B, NCKAP1, IGF1R, MED13, RSRP1, ZNHIT6, PSMB8, PDCD4, TAX1BP1,
MAP3K2, TNIP1, PROM1, VCP, HSPH1, ACTG1, CNN3, EXPH5, CCAR1, ZFP91, SETX, USP8, JMJD1C, PRDX6, MYH9,
NAMPT, NPEPPS, SERP1, GLG1, FRMD4B, MKRN1, TFDP2, PUM2, RNF152, EIF4G1, DAP, ACSL3, NKTR, SERTAD1,
UHMK1, ZZEF1, PPL, ISG20, EP300, RHBDD2, TRAF4, NUFIP2, AHI1, JTB, ID1, ITPKC, RMDN3, DNAJA1, REL, FLII,
CAB39L, TBC1D9B, CHD1, TRAM1, SOCS6, CTBP2, SPINT2, BPTF, CCDC186, SLC12A2, ARPC2, TUT7, SP3, KDM5B,
HNRNPD, GALNT12, RBM47, PRDX1, CLUH, CAMK2G, ABHD11, SLC15A2, UBXN4, DTX2, TANC1, TMSB10, NSD1, CTSC,
ADNP, FUT3, TRIB2, ARHGEF28, KDM7A, SH3GLB2, RBM5, DAAM1, DNAJB1, PPP4R3A, DNMT1, KIAA1109, NFE2L1,
PLEKHA7, NIPBL, RAD21, AFF4, SYNGR2, YY1, SMARCC1, ATP2A2, OXR1, PCBP2, SCARB2, THRAP3, CARD19, NET1,
HLA-DRB1, GSPT1, PTP4A2, TMEM259, STAT3, HSPA1B, SP110, PLA2R1, DLG1, LDLRAP1, HSPA1A, CHD9, GPI,
SIPA1L1, PITX1, BAG1, PSAP, CEACAM6, CACUL1, ARF1, N4BP2L2, JUN, SUN1, IFIH1, MAGI3, NFE2L2, CAPNS1, RABL6,
CDH1, AVPI1, IL6ST, RXRA, ZNF148, SLC20A1, TACC1, ENTPD5, OASL, NCOA3, CDC42BPB, UBTF, ARID2, KLHDC2,
HDAC7, BIRC6, CSNK1A1, SWAP70, BTG1, COL4A3BP, DNMBP, NR1D2, GUK1, ALDH3B1, B2M, SLC9A3R1, SEC62,
AKR1A1, CLTC, GOLGA3, KDM3B, SF3B2, UBE3A, DENND2C, UNC93B1, AFDN, CEP350, GADD45B, BAZ1B, ZBTB38,
PIK3IP1, LARP1, SMARCA4, HSPA8, NCOA2, CD36, ITSN2, TOMM20, ZKSCAN1, PTPRU, PLEKHS1, ZNF638, ARAP2,
HP1BP3, CNBP, SCAMP2, F11R, RBBP6, SORBS2, SPARCL1, YWHAB, CASP3, SLC25A3, BRD4, SECISBP2L, CYFIP2,
GTF2F1, H3F3B, PTPRF, FAM102A, CXCL17, HEBP2, MECOM, CEMIP2, NFX1, ATMIN, WIPI2, COBLL1, FOXA1, S100P,
HNRNPH1, BSPRY, HUWE1, TTC9, SPSB3, TSPAN6, IBTK, KMT2E, PDZD8, PSMD1, TENT5A, TMEM248, TMEM160,
BTAF1, CXCL16, TP53BP2, U2SURP, RNH1, CCNL1, EFCAB14, DDX42, TOP2B, CARMIL1, SLC39A7, ORMDL3, MYL6,
MUC20, PSMG3, TRAK2, C4orf3, QARS, ATP1B1, GNPTAB, LRIG1, BMPR2, HNRNPM, SHISA5, DIS3, DTX3L, SESTD1,
SSRP1, PDS5A, RNF114, SOD1, MIA3, ERBB2, EPS8L1, DCAF7, PRR15, CBR1, VCL, MYO5C, ZNF106, PNPLA2, MED13L,
MLLT6, CCNI, PTK2, PFKL, KCMF1, ANKRD17, ALDH9A1, RIPK1, FBXO32, PDLIM5, MFSD6, RBM3, GABRP, PPP1CC,
MKL2, ATP6AP1, TMOD3, MYO5B, PRPF4B, MATR3, CPNE3, SLC44A4, PRRG4, ALDH2, EPHX1, REEP5, YTHDC1,
TRIM24, MMP10, JAK1, FBXW5, CAPN2, TRAF3IP1, 6-Mar, DDR1, RALBP1, RAI1, VAMP8, SPINT1, ECPAS, FAM84A,
FHL2, STAU1, YME1L1, PATJ, ESCO1, EPB41L1, SIX1, IGF2R, ITGB1, ZMYND8, TUBB4B, GBP1, ZMYM4, SLMAP, RTF1,
UGT2A1, ADD1, EGFR, ZDHHC20, AUTS2, RAB7A, FGD5-AS1, VSIG2, RNF115, PKN2, BRWD1, TAB2, RBM33, PODXL,
B4GALT4, UBE2D3, ASPH, WWTR1, MTCH1, CTCF, RAB13, ASAH1, C16orf72, TRIM8, ALAD, FUBP1, USP7, RUNX1,
LUC7L3, OSBP, AGRN, KDM5A, IK, PSMD7, TRIM56, RHOA, CASC4, SAP18, RDH10, PTPN11, CRYM, C1orf21, DYRK2,
ETF1, FAU, STK38, HSPA4, TMED4, NUCB2, NSD3, GPR107, HLA-F, SMG1, MBP, ELF1, PYGL, S100A13, CAMK2D, PLEC,
UBR4, ACIN1, SEC63, ZC3H13, COPA, DUOXA1, ESRP1, TMEM30A, JUND, BICDL2, ENSA, BCAP31, PRPF38B, CYTH1,
EIF1, SUPT5H, NAA50, EI24, UBE2K, GNAS, NEDD4L, WDR45B, MYL12B, TRIP10, MPZL2, CALCOCO2, EIF3D, CLTA,
PDXK, SGSM2, TPD52L1, PRRC2B, PPM1G, SOX2, GRN, ZFHX3, PUM1, MAPKAPK2, ARID5B, HK1, UBXN1, SDC4,
CYB561, OSBPL8, KIAA1324, KPNB1, LAMP1, CIAO1, DNAJA4, MAGED1, NEAT1, AMOTL2, TMED3, FUS, CD81,
DIAPH1, ABCF1, ARCN1, NAPA, TNRC6B, TRIM29, UGDH, SLC3A2, CASP7, SIRT7, KLF3, SPATS2L, PIGT, IST1, CIR1,
PPP1R12A, CDV3, ATF7IP, ALDH3A2, CTTN, MYO6, VGLL4, RAB2A, SMC5, CPD, ENAH, TM9SF2, RANBP9, ENTPD4,
CDC37, RSF1, NPTN, ATXN7L3B, FBXO34, XRCC5, HNRNPF, PHACTR4, CBX4, FAM114A1, UBA52, PARD6B, SLU7,
SRSF4, DNAJC5, DYNC1LI2, POLR2A, PHIP, KIAA1522, MTMR10, CTNNB1, METAP2, PI3, ARSD, KLF4, HNRNPR, TTC19,
EXOC1, TRAP1, STARD7, KPNA3, OTUD7B, TSPAN14, RAB31, EPS8, VEGFA, NECTIN4, RABGAP1, NUCKS1, EID1,
HTATSF1, TMEM150C, HSPA9, PRR14L, PAPOLA, SCNN1A, PSD3, ZNF750, PAFAH1B1, SPTLC2, SEL1L3, SDF4, CLIC6,
RC3H1, MRFAP1, UAP1, MCL1, TJP3, CDCP1, TSPAN13, ATP8B1, OS9, SMARCC2, RIF1, SIN3A, VTCN1, SEC14L1,
RSBN1L, SLC16A9, ANXA2, RBBP7, LLGL2, USP47, NONO, MAGED2, CNP, LMNA, SEC31A, CCNO, IDH2, SYF2, EIF3H,
NRDC, BAG5, SYNCRIP, HADHA, PRKAR2A, CLDN1, PFKP, MAP7, ANPEP, UGCG, DLG5, AQP5, PPM1K, S100A6, YAP1,
DICER1, NPC2, MAVS, UBAP2L, PICALM, BBX, CTR9, GNE, TCEA3, TMF1, FTL, PIK3R1, NUP50, CLK1, CHMP3, ERBIN,
TMEM123, BRI3, EIF3G, GPBP1, EYA2, KRT19, LY6D, TM4SF1, MAL2, CDC5L, MISP, OSBPL2, LMTK2, RNF10, SPEN,
WASF2, GIGYF2, GOLGA2, CDK13, CHD8, ARPC5, SLC6A14, GNS, SLC4A11, THOC2, SRP14, SH3GLB1, ID2, TMC5, CIB1,
LRRC8A, IRF1, PDIA3, HNRNPAB, STUB1, LONP2, TOP1, RPN2, VPS28, PLCE1, WAPL, LGALS3, RDX, SRRM1, ADAM10,
SMARCA5, PJA2, MVP, RBFOX2, GNPNAT1, SMC1A, CDK12, TCIRG1, TSPYL2, ZDHHC3, CALR, ARRB2, EFNA1, MTPN,
EIFSB, ERLEC1, KARS, KRT4, ATF6, NDUFV1, IDH1, DGKH, RND3, CAPN5, TACC2, ATP5F1B, UBE2R2, KIAA0232, LPP,
STEAP4, KLF2, PSMB4, GLTP, LCN2, CYP2J2, AP1G2, PADI1, 7-Sep, MPV17L, CCDC47, SPAG9, UBE2J1, H1F0, TJP1,
PTPRK, B4GALT1, REST, DDX21, ATP5IF1, ABCD3, TRIM38, SMAGP, CD47, SLC5A8, ERGIC3, B4GALT5, NDUFB10,
BCLAF1, S100A14, MLF2, RAC1, CANX, SLPI, PUF60, RYBP, CGN, KIAA1191, GDF15, OAZ1, ESRP2, TTC37, SCP2, YPEL5,
BDP1, MIA2, RBMX, GGNBP2, TFRC, GNL3, PRPF8, BCL2L11, H6PD, ATP6V1G1, C11orf58, MCU, RASEF, RNF7, MLPH,
PAQR4, PTPRA, ARID4B, ARFGEF3, PPP2CA, CDC42BPA, MSI2, LAMP2, EFEMP1, TKT, SRSF3, GAN, LITAF, CKB, SLC2A1,
CAND1, DUSP10, AMD1, TMEM205, CXXC5, SEPHS2, ZNF24, ANKRD13A, PNRC1, EMC10, DDX1, FOS, UBE2H,
ADGRG1, HIPK3, MEA1, SSU72, AES, HNRNPC, SDCBP2, FTH1, CAPN1, ATP5F1A, CAMK1D, RAB14, EIF3K, FAM129A,
WASL, GCC2, SORT1, NR3C2, KMT2A, TOR1AIP2, STK39, HIPK2, LARP4B, GALNT7, TMEM50A, STAT6, PBX1, CD44,
COPB2, P4HB, RNF145, MET, HOMER2, NORAD, SNX6, DHX9, MBNL1, ZFR, CREB3L2, IRF2BP2, HPGD, FOSL2, PLCB4,
CRK, SEC61A1, PIM3, GPC1, RTN4, LYPD2, TAOK1, JUP, DNAJC3, RPN1, AP2M1, AKR1C3, PDCD6IP, NANS, GSR,
TUBA1B, ASCC2, PLAC8, DYNLL2, CUX1, CST3, DLG3, TMEM30B, ACTN4, ANAPC5, LYNX1, MUC13, CDC42EP4,
CTTNBP2NL, CKAP4, UPF1, BAG6, PSMA3, PSMB1, EDF1, PGRMC1, LDHB, GNB1, GNG12, SLC25A6, CTSD, EIF4G3,
PSMD12, IRAK1, KRAS, PPIG, RTN3, NFIC, RAI14, TMEM59, SCIN, PSMA4, TMEM165, IDO1, SERINC3, EIF4A2, VPS4B,
TMEM213, TMX4, LAPTM4B, PGM2L1, CD24, ACBD3, SET, EIF6, IQGAP1, TPM4, SEMA3C, TUFM, DDB1, PSMB7, CYC1,
IFITM2, AUP1, ARF6, ELL2, GAPDH, SRPRA, DAZAP2, TMEM219, PER2, MSMB, DEDD2, RAP2B, RAB1A, TLN1,
PPP1R15A, ABHD2, LPCAT4, FOSB, ASRGL1, CAPN13, DDIT3, RAB5A, KHDRBS1, CD55, ARL6IP4, TOB2, COX5A,
TMED10, MAPKAP1, TALDO1, HCLS1, USP22, KCNK5, GRB2, TCP1, PSMB3, PCBP1, CMTM6, H2AFJ, CD46, USO1,
CSRNP1, SERINC1, NFIA, SGK1, PRSS8, TERF2IP, NDUFA2, PSMD2, HM13, CD164, UBQLN1, BCL6, ACADVL, EEF1A1,
ETS2, NOLC1, ID3, CHP1, REEP3, G3BP2, LASP1, YWHAZ, NEDD8, CHL1, EMP2, ATP6V0E1, PALLD, MIDN, EPHA2,
SERINC2, SPINK5, ANXA5, PSMC3, METTL7A, SPRR3, NEBL, WFDC2, LDHA, CHMP2A, CCDC69, TMCO1, WDR1,
RASSF9, ECHS1, PLEKHB2, EMC4, SSR4, TUBA1C, SGPL1, ENO1, ICMT, ZNF185, HSP90B1, PSMC5, IER3, C6orf62,
IQGAP2, SRSF7, ALDH1A3, GRHL2, CSTB, PHB, FAM120A, G3BP1, ARL1, BAG3, SCPEP1, TSPAN1, SKP1, TMED9, TPI1,
EIF4EBP2, KRT23, UQCRC2, CLDN7, LAMTOR5, PCDH1, NFKBIZ, PDIA4, SLC38A1, STK17B, CFL1, PSME2, MXD1,
TSC22D1, KCNE3, KLF6, SRI, ZFP36L1, FUT2, 2-Sep, CHMP1B, PTBP3, ZFP36, RHOC, AKR1C1, CYB5A, LAPTM4A,
ZNF652, VMP1, TRA2B, TAF7, CDKN2AIP, ZFP36L2, PPA1, SELENOP, BTF3, ZNF217, EIF4A3, RHOV, COX14, GNG5,
PRDX5, MLEC, SMIM14, CCT6A, DYNLL1, YBX3, EIF4B, PMAIP1, GPT2, UGP2, SFN, ACTR2, ARRDC3, NOS2, ANXA1,
SNRPD2, JUNB, PIK3R3, SERBP1, TOB1, GPX4, PSMB6, C1orf116, IKZF2, TSHZ2, XRCC6, ADAM9, MDH2, BHLHE40,
ATF3, TPM3, SLC44A2, TSPO, PTMA, ZFAND2A, ATP6V0B, LRP11, IER5, IER2, CAP1, EFNB2, DUSP5, DHRS9, ASS1,
PDIA6, IL1R1, SOX4, LAMB3, HSBP1, GHITM, SOCS3, BLVRB, ALPL, IVNS1ABP, PPIB, HNRNPK, A4GALT, COX8A, CA12,
S100A10, ATP1A1, FMO2, MSLN, HES1, CLU, PPDPF, SH3BGRL3, HSPB1, HSPD1, CHKA, CTSH, KLF10, NFKBIA, CEBPD,
DSTN, TIPARP, MYL12A, CYP2F1, NR4A1, SLC25A25, ST6GAL1, DUSP1, UPK1B, COX7C, GPRC5A, CDKN1A, C19orf33,
FAM107A, KRT7, EMP1, IRS2, INSIG1, S100A11, KRT6A, EGR1, RARRES1, AKR1B10, AGR2, MAFF, COX5B, BTG2, GLUL,
KRT18, ACTB, ANKRD36C, NTS
Squamous Cells (all)
S100A4, NR4A2, UCA1, TIMP3, SLC38A2, KRT4, CPA4, WARS, MX1, VEGFA, AQP3, SLC5A3, GNE, GBP1, ALDH3A1,
RNF19A, FAM102A
T Cells (all)
HLA-B, HLA-C, TMSB10, TUBB4B, HSPH1, B2M, HLA-A, DNAJB1, NACA, GZMB, HSPA8, HLA-E, GNLY, ACTG1,
HSP90AA1, EIF4A3, FOS, GADD45B, BRD2, HSPA5, ODC1, MX1, FAM107B, XAF1, KLF6, ATF3, GZMA, HSP90AB1,
SP100, MYLIP, TMBIM6, SERTAD1, PRNP, HLA-F, MYL6, CD3D, SAP18, OAZ1, IFI44L, DYNLL1, ZC3HAV1, CD74, PNP,
EEF1D, ADAR, GNAS, IL2RB, PPP1R15A, RNF213, ATP5F1B, RAC2, UBB, UBC, VIM, HSPA1B, VCP, DEDD2, TAP1, ACTB,
ZFAS1, ISG20, CIRBP, MCL1, IFI16, AHSA1, RHOA, LCP1, CORO1A, LCK, IER5, SRGN, IFI6, GBP2, SLAMF7, EIF4G2, LDHA,
HSPA6, BCL2L11, PSMB9, PSTPIP1, ENO1, HNRNPA2B1, SARAF, ARPC2, FLNA, STAT1, KLF2, APOL6, LCP2, CDV3,
C16orf72, IRS2, SNHG7, EEF2, IVNS1ABP, EZR, EIF2AK2, SUN2, GAPDH, ISG15, TOP1, PRF1, LAG3, NFKBIA, SNRNP200,
TXNIP, ARHGAP30, STK17A, GBP1, PSMB4, UBA52, ITGAL, C6orf48, PREX1, CBX4, FAM53C, JUN, CSRNP1, SQSTM1,
LNPEP, CFLAR, TAGAP, ID2, AHNAK, DUSP4, CALR, IFITM2, SLA, 9-Sep, CLK1, APOBEC3G, PABPC1, HCLS1, ATP2A2,
ARRDC3, IKZF3, LPIN2, SAMD9L, TUBA4A, HSP90B1, SEMA4D, NCL, RYBP, GTF2B, PSME1, HNRNPH1, EHD1, ATF4,
ACAP1, XIST, CANX, STOM, TLN1, NOP56, PPP1R16B
AZGP1 high Goblet Cells
AD000090.1, CYP1B1
BEST4 high Cilia high Ciliated Cells
TBC1D8, PARP14, ETV6, IFI44L, RNF213, MX1, PROM1, IFI6, IFI44, ABCA13, TNFAIP2, STAT2, PIGR
Early Responsive FOXJ1 high Ciliated Cells
DDX3Y, AD000090.1, PIGR, ABCA13, RBM3, IL33, GSTA2, PROM1, OMG, EPPIN, AL357093.2, ALDH1A1, ODF3B, AGR3,
NFE2L2, CXCL17, C12orf75, UGT2A1, TDRD1, TMEM67, S100A11, ACTB, PRSS23, USF1, GRAMD2B, AQP4-AS1,
HSD17B13, RHBDD2, TBC1D8, PROS1, CTSH, SERPINB1, LRRC36, IFITM1, SFPQ, PLAC8, SLC6A6, STOM, FUS, ACADM,
EGR1, VIM, DNAH5, EIF5A, GAPVD1, TNFAIP2, ACTG1, F11R, IGFBP2, GABPB1-AS1, DNAAF1, ELF3, TUBA1A, VNN3,
FTO, ZNF326, IFI44L, SNTB1, SAT1, RAB34, NDUFB9, DAZAP1, PSMB3, TPR, TCTEX1D4, B9D1, ISG15, TMEM14B,
MAPK15, DDX24, TMEM154, ATP5PB, COLCA1, METTL27
Early Response Secretory Cells
PARP14, MX1, TNFSF10, RNF213, IFI44L, TNFAIP2, IFI27, IFITM1, HERC6, MSMB, IFI44, IFITM3, STAT1, ISG15, S100A4,
IFIT1, IGFBP3, EPSTI1, CCDC80, OAS2, MDK, ALDH3A1, SLC6A14, IFI6, EIF2AK2, AQP3, TYMP, TM4SF1, XAF1, HLA-
DRB5, MX2, KRT4, SP100, CEACAM5, TRIM22, ITGB8, BST2, RARRES3, PARP9, PI3, ARHGAP18, APOL6, MUC4, SPRR3,
TGM2, SARSCoV2-3prime, VPS13C, DDX60, RBM3, MACC1, OAS1, DDX58, OAS3, CCND1, IFIT3, RSAD2, SLFN5, ADIRF,
LAP3, SLK, GALNT5, PER3, LY6E, STAT2, ASS1, MYOF, ADH7, PLSCR1, APOL1, ADAR, FLNB, TMOD3, S100A10,
C6orf132, UBE2L6, EFHD2, ANKRD36C, SLC6A6, KIF1C, SAMD9L, F2R, AKR1C2, ST8SIA4, RBM25, ZNFX1, SLC28A3,
PRDX1, SECTM1, OPTN, SAMD9, LMO7, AQP5, CEACAM6, CMPK2, FAM107B, PSME1, APP, TP53I11, FBP1, CYP2S1,
TAP1, S100A16, CLEC7A, PLXNB1, IFIH1, XIST, PGD, TAPBP, SDC1, PADI1, RCC2, SLC12A2, TPR, NQO1, PTMA, AXIN2,
ALDH1A1, CCDC88C, TRIB2, CMYA5, PNISR, NINJ1, PTGES, CPLANE1, FBXW4, PHACTR2, LGALS9, AHI1, NEAT1, CD82,
IFITM2, ALOX15, NSMCE4A, WARS, TCERG1, ATP7B, B3GALT5, NFE2L2, ETV6, DHRS3, PSME2, CYLD, SMCHD1, NCL,
NLRC5, OGFRL1, ANP32B, CTSS, DDX60L, PTPRZ1, KIAA1551, SH3RF1, CAST, CD151
FOXJ1 high Ciliated Cells
AD000090.1, DDX3Y, GSTA2, SLC6A6, RBM3, PROM1, FTO, TBC1D8, SFPQ, IL33, STOM, AL357093.2, PIGR, SLPI,
STEAP3, ACTG1, MAPK15
MUC5AC high Goblet Cells
PARP14, IFI44L, IFI44, MX1, SP100, FER1L6, STAT1, HERC6, RNF213, EIF2AK2, DDX58, IFIT1, EPSTI1, XAF1, AHI1,
SLC28A3, MX2, IFI27, IFIT3, TNFAIP2, TNFSF10, ISG15, TRIM22, PARP9, PLEKHH1, GK5, LMO7, RBM25, XRN1, OAS2,
SFPQ, STAT2, MTUS1, TRIP12, ADAR, TCF25, IFI6, UBA6, FAM107B, DDX60, USP15, DDX24, CP, PDXDC1
Interferon Responsive Ciliated Cells
TBC1D8, STAT1, IFI44L, MX1, IFITM3, ISG15, IFI44, PARP14, IFITM1, RNF213, RBM3, OAS2, OAS3, SLC6A6, SAT1,
RSAD2, ATP12A, GCLC, OXTR, PROM1, HERC6, LAP3, IFI27, IFI6, TRIM22, MX2, IFIT1, CMPK2, TMC5, ADAR, ETV6,
PLSCR1, DUOX2, IFIT3, AKR1C2, ADH7, STAT2, LY6E, HLA-E, PARP9, IRF1, SPTBN1, COLCA1, SAMD9, ISG20, CBR1,
CBR3, PGD, APOL6, PFKP, MYOF, CTGF, SLC37A1, PALLD, RHBDD2, DDX60, OGFR, AD000090.1, IFI16, PIGR, ABCA13,
ANXA1, UBE2L6, XAF1, SDC4, OAS1, SPARCL1, DUOXA1, GBP4, LGALS3BP, STOM, EIF5, SAMD9L, AP2B1, FUS, SP100,
HMGXB3, TAP1, APOL1, ALDH1A1, SLFN5, DTX3L, MDM2, GBP1, DDX5, HELZ2, EXOC3, RTP4, FAM107B, STAT3, REC8,
MAP3K2, TMEM173, PLXNB1, SLC23A2, ACTG1, RBM25, TRAF4, RNF19A, PLEKHA4, GNA14, PRDX1, DDX24, LYN,
CYB561A3, HNRNPDL, TAPBP, CHST9, CTNNAL1, SPG7, GBP5, ATP1B1, GSR, CDH1, SCO2, CAMSAP1, APP, UBR4,
MYO1B, LAMP3, TCERG1, MYO5B, SLC25A6, DLG5, TNFRSF19, TMEM123, TRIM2, ATP6V0A2, UNC93B1, EEF2K,
MTUS1, B3GNT5, TRIM8, GAPDH, LIMK2, PELI1, ATP1A1, CLUH, PER1, ZFAND5, SHISA5, CLINT1, CES1, PARP12, ATF4,
JAK2, CCPG1, TXNRD1, ELF3, TRIM69, LARP1, EZR, RBP1, SGSM2, CCDC189, PLXNB2, DDX58, KIAA1217, WDR90,
EPSTI1, HK1, TNFAIP2, TRAK1, GCLM, TSPAN3, POMT2, SRSF5, HLA-C, HNRNPD, PSMB9, FRMD4B, CD38, SLC4A11,
GSTA1, OPTN, PPP1CB, SLC26A2, BUD23, SRSF2, AKR1B10, PYGL, SERPING1, CEP170B, APEX1, DNHD1, VNN3, ITM2B,
AGRN, HNRNPR, GLUL, ATP6V0A4, KIF2A, SNRNP200, KIAA1522, PDXDC1, RHOU, BRD2, CIRBP, ANKRD28, ACTB,
PSME4, DIAPH1, XRCC5, EIF2AK2, APLP2, GRHL2, XRN1, PRPF8, HNRNPK, IDI1, SP110, CLDN1, SERPINB1, CRACR2B,
AQP3, CSNK1G2, IFT172, CTNNB1, PRR14L, PLAC8, STARD7, CTSS, RAB31, CCNL1, TRAP1, ZNFX1, EHD4, PPP1R15A,
GPX2, TMEM67, OSBPL2, UACA, CASC4, UTRN, LMO7, DRC3, WARS, DSP, WDR26, LPGAT1, PEBP1, PERP, OASL,
CELSR1, TTC21A, TACSTD2, EIF4G2, IFIH1, KIAA0232, ANXA11, ATP2B1, NCL, POLR2A, TALDO1, PSMB3, DIAPH2,
RHOA, GSN, HDGF, DDX42, RERE, MARK3, PPM1L, SFPQ, ILF3, PIAS3, MYL12A, GGT6, ESRRA, DUS1L, SMG7, LRRC74B,
HLA-F, CTSH, CDCP1, SORT1, PSME2, PLA2G16, PRSS23, CLUAP1, ASRGL1, DYNC1H1, ZNF664, HIPK2, TPGS2, ARMH1,
NDRG2, SWAP70, IFITM2, PDIA3, SF3B1, MKRN1, ST6GALNAC1, EPCAM, SLC2A1, SLC25A3, LRP10, TMEM45B,
C16orf72, JUN, CFLAR, TXNIP, IDS, GSTA2, WDR1, RBM5, LZTFL1, MYL12B, CPEB4, SOD2, ALDH3A1, RBM47, SEC63,
MUC1, DNAAF1, UBR5, KLF6, KLF4, MACC1, ANKRD54, RP1, UCP2, CAPN2, TMEM190, YWHAZ, CXCL17, CCDC88C,
CTSB, ARHGAP32, TMEM245, SRSF3, METTL7A, RARRES3, UBC, B3GNT7, PRKAR1A
SCGB1A1 high Goblet Cells
SCGB1A1, IFI6, MX1, BPIFA1, IFI44
SERPINB11 high Secretory Cells
TSIX, ABCA13, SYNE2, ADAM28, CPLANE1, STAT1
CCL5 high Squamous Cells
CCL5

TABLE 5
Common Differentially Expressed Genes Between SARS-CoV-2 RNA+ Cells and Bystander Cells. Related
to FIG. 6. log2 fold change between SARS-CoV-2 RNA+ cells (high, positive values) and matched
bystander cells (low, negative values). Columns: detailed cell types with at least 5 SARS-CoV-2 RNA+ cells

| | | BEST4 high | | | | | Interferon | KRT24 KRT13 |
|---|---|---|---|---|---|---|---|---|
| | | Cilia high | BPIFA1 high | Developing | FOXJ1 high | | Responsive | high |
| | AZGP1 high | Ciliated | Secretory | Ciliated | Ciliated | Goblet | Ciliated | Secretory |
| Gene | Goblet Cells | Cells | Cells | Cells | Cells | Cells | Cells | Cells |
| SAA1 | −0.6744 | −0.0122 | 0.0946 | −0.3336 | 0.0002 | 0.0725 | −0.8702 | −0.1021 |
| CD74 | −0.4089 | 0.0894 | −0.0986 | −0.1087 | −0.2153 | −0.5348 | −0.6455 | −0.3701 |
| HLA-DRB1 | −0.2059 | −0.1306 | −0.0247 | 0.0680 | −0.0434 | −0.4507 | −0.8144 | 0.1716 |
| HSPA1A | 0.2471 | 0.1585 | −0.1646 | 0.2672 | 0.3755 | −0.7105 | −0.0177 | −0.5203 |
| HLA-DRB5 | −0.2421 | −0.1566 | 0.0000 | −0.0117 | −0.1341 | −0.2224 | −0.8156 | 0.2563 |
| HLA-DRA | 0.0474 | 0.0426 | 0.1255 | −0.2572 | −0.4837 | −0.5443 | −0.5872 | 0.1083 |
| APRT | −0.2052 | 0.0705 | −0.1822 | −0.0415 | 0.1752 | −0.0141 | −0.3301 | 0.2675 |
| MMP10 | −0.3828 | −0.0005 | 0.0000 | −0.0239 | −0.0284 | −0.7655 | 0.0467 | 0.5288 |
| CCDC171 | 0.0002 | 0.0570 | −0.0544 | −0.0473 | −0.3218 | −0.1605 | −0.0449 | 0.0067 |
| CDKN1A | 0.0651 | 0.0183 | 0.0634 | 0.1365 | −0.2791 | −0.0589 | −0.3692 | 0.5566 |
| RARRES1 | 0.2038 | −0.0142 | −0.2647 | −0.0507 | −0.0748 | 0.0489 | 0.0411 | −0.8124 |
| LDLRAD1 | 0.0380 | 0.2313 | −0.0255 | −0.0388 | −0.6062 | 0.0000 | −0.5857 | −0.0211 |
| HTATSF1 | −0.1411 | −0.1932 | 0.0336 | −0.0988 | 0.0685 | −0.3339 | −0.0619 | 0.2288 |
| FMO3 | 0.2362 | 0.3503 | 0.0000 | −0.2556 | −0.9620 | −0.2224 | −0.4546 | −0.4644 |
| EIF4G2 | −0.1668 | −0.2075 | −0.1089 | 0.1463 | −0.3792 | 0.0885 | −0.0065 | 0.4219 |
| HSPA1B | 0.1059 | 0.2751 | −0.0448 | 0.1592 | 0.4525 | −0.8395 | 0.1452 | −0.5783 |
| TP53BP1 | −0.0382 | −0.1827 | −0.0086 | 0.0000 | −0.2863 | −0.0619 | −0.3943 | 0.0062 |
| PLAT | −0.0408 | −0.0277 | 0.0089 | −0.0298 | −0.0189 | 0.0000 | 0.0275 | −0.0280 |
| FCGBP | −0.4236 | 0.0195 | −0.0610 | −0.0298 | −0.0564 | −0.0589 | 0.0033 | 0.0138 |
| CCDC113 | 0.0967 | 0.3387 | −0.1325 | −0.1551 | −0.5741 | −0.0317 | −0.1573 | −0.0280 |
| UGDH | −0.0134 | −0.1728 | −0.1740 | −0.0204 | −0.3284 | −0.0280 | −0.0403 | −0.1065 |
| NWD1 | −0.0366 | 0.0346 | −0.1440 | −0.0612 | −0.5093 | −0.0133 | −0.3888 | 0.1894 |
| RPS6 | −0.3443 | 0.3805 | 0.0482 | 0.2593 | −0.2710 | −0.2950 | −0.2115 | 0.3504 |
| FAM216B | −0.0475 | 0.5789 | −0.0589 | −0.2752 | −0.6928 | −0.0473 | −0.3163 | 0.0348 |
| RPS19 | −0.3125 | 0.2434 | −0.0981 | 0.1547 | −0.2879 | 0.0151 | −0.0606 | 0.4235 |
| SEC14L3 | −0.0136 | −0.7305 | −0.0633 | −0.0175 | 0.1176 | −0.0780 | −0.3245 | −0.0141 |
| LGALS8 | −0.2305 | −0.1087 | −0.0352 | −0.0114 | −0.2586 | −0.1417 | −0.2210 | 0.0911 |
| TCP1 | 0.1380 | 0.1721 | −0.1234 | 0.0157 | −0.2668 | −0.1349 | −0.1298 | 0.0589 |
| PGM2L1 | −0.2868 | 0.2718 | −0.0610 | −0.0055 | −0.0460 | −0.3339 | −0.0125 | −0.0485 |
| NFIA | −0.2468 | 0.0011 | −0.1240 | −0.0672 | −0.0651 | −0.0704 | −0.1251 | 0.1541 |
| MTURN | −0.3089 | 0.2008 | 0.0089 | 0.0282 | −0.1036 | 0.2204 | −0.1769 | 0.1420 |
| TXNL4A | −0.0575 | 0.0304 | −0.0255 | −0.0348 | −0.0709 | −0.0825 | −0.3751 | 0.0000 |
| CCNO | −0.3758 | 0.1632 | −0.0265 | 0.0628 | −0.0394 | −0.1375 | −0.1374 | 0.1660 |
| ATF3 | 0.9592 | −0.0245 | −0.2297 | 0.0719 | 0.0562 | 0.0406 | −0.2286 | −0.2168 |
| HLA-DPA1 | −0.0643 | −0.0277 | 0.0179 | 0.0406 | −0.2333 | −0.1664 | −0.5263 | 0.0614 |
| TSGA10 | −0.0495 | −0.3163 | 0.0173 | −0.1284 | −0.2705 | −0.0133 | 0.0858 | −0.0605 |
| ERLEC1 | −0.1697 | −0.0440 | −0.1984 | 0.0109 | −0.3190 | −0.0825 | −0.0747 | 0.0371 |
| SCGB1A1 | −0.5543 | 0.1167 | −0.0423 | 0.0584 | 0.0188 | 0.0647 | −0.1532 | −0.1043 |
| ALOX15 | 0.0567 | 0.3814 | −0.1751 | 0.0725 | −0.1184 | −0.5944 | −0.3289 | 0.3864 |
| BRD7 | −0.0215 | −0.0122 | −0.0780 | −0.0647 | 0.2306 | −0.2102 | −0.0165 | −0.0436 |
| XBP1 | −0.1955 | 0.2899 | −0.2522 | −0.0362 | 0.1830 | −0.1806 | −0.3536 | 0.1686 |
| PLCB4 | −0.0686 | 0.5823 | −0.1575 | 0.0053 | −0.2528 | 0.0083 | −0.4001 | 0.2021 |
| BRD8 | −0.0272 | 0.6447 | −0.0695 | −0.0171 | 0.0452 | 0.0121 | −0.1669 | −0.2405 |
| BAZ1B | −0.0441 | −0.1531 | −0.0339 | 0.0109 | −0.1538 | −0.3344 | −0.2513 | 0.1541 |
| HSPH1 | −0.1097 | 0.2680 | −0.3273 | −0.0494 | −0.0437 | 0.0202 | −0.5826 | 0.2768 |
| CEP83 | −0.0295 | −0.1928 | −0.0610 | 0.0275 | −0.1373 | 0.0142 | −0.2890 | −0.0330 |
| RSRP1 | −0.1373 | 0.0259 | −0.0080 | −0.0378 | 0.1170 | −0.4811 | −0.1156 | 0.2523 |
| ADH1C | −0.2926 | 0.3189 | −0.0732 | 0.2193 | −0.1176 | −0.4097 | −0.2568 | 0.2873 |
| PPP1R7 | −0.2754 | 0.1662 | −0.0089 | 0.1255 | −0.1784 | −0.0418 | −0.2105 | 0.1390 |
| SMC4 | −0.0837 | −0.2010 | −0.1758 | 0.0960 | 0.0422 | −0.4330 | −0.0958 | 0.3663 |
| DOCK1 | −0.1820 | 0.1842 | −0.0721 | 0.0172 | −0.0315 | −0.1375 | −0.0159 | −0.1037 |
| DZIP3 | 0.0356 | 0.3483 | 0.0078 | −0.2080 | −0.5198 | −0.1520 | −0.3601 | 0.0338 |
| ECI1 | −0.1441 | −0.0245 | −0.0177 | 0.0172 | −0.3149 | 0.0825 | −0.2335 | 0.3312 |
| NDFIP1 | −0.2969 | 0.1565 | −0.0834 | 0.0056 | −0.2680 | 0.1829 | −0.0151 | −0.0690 |
| ACAA1 | −0.1585 | −0.3307 | −0.0721 | −0.0818 | −0.0169 | −0.2224 | −0.0654 | −0.0369 |
| TOR1AIP2 | −0.1316 | −0.1689 | −0.1575 | 0.0172 | −0.1789 | −0.2464 | 0.1125 | −0.1099 |
| RPS27 | −0.0283 | 0.2434 | −0.4887 | 0.0944 | −0.0994 | −0.2086 | 0.1141 | 0.1472 |
| ACTR3 | −0.0968 | 0.1908 | 0.0693 | −0.0055 | −0.2443 | −0.2479 | −0.1188 | −0.0444 |
| ZFC3H1 | −0.0097 | 0.0229 | 0.1168 | −0.0632 | −0.0726 | 0.0662 | −0.0266 | −0.2675 |
| PNRC1 | −0.0673 | 0.3461 | −0.0834 | −0.0233 | −0.1621 | −0.2605 | −0.0472 | −0.0060 |
| OSBPL6 | −0.0727 | −0.1863 | 0.0704 | 0.0117 | −0.5214 | −0.0317 | −0.4244 | −0.0418 |
| KIAA0100 | −0.2741 | 0.1343 | −0.0265 | 0.0628 | −0.4544 | 0.0740 | −0.1186 | −0.1580 |
| IGFBP5 | −0.0340 | −0.0137 | −0.0896 | −0.0763 | −0.3937 | −0.0473 | −0.3424 | −0.0071 |
| UPF3B | −0.0978 | 0.1857 | −0.0352 | 0.0960 | −0.1194 | −0.3326 | −0.0654 | −0.0867 |
| HLA-DQB1 | −0.0624 | −0.0412 | −0.0092 | 0.0058 | −0.2079 | −0.1806 | −0.4506 | −0.0211 |
| CARS2 | −0.2361 | −0.0045 | −0.0365 | −0.0175 | 0.1377 | −0.2895 | −0.0285 | −0.1312 |
| PLEKHA5 | −0.1775 | 0.0369 | 0.1045 | 0.0109 | −0.2034 | −0.3799 | 0.1293 | 0.2630 |
| LRRC46 | −0.0131 | 0.1710 | −0.0610 | −0.0109 | −0.2588 | −0.0159 | −0.2680 | 0.0348 |
| GCHFR | −0.3629 | 0.1032 | 0.0000 | 0.0058 | −0.0163 | 0.1255 | 0.0468 | −0.2168 |
| NISCH | −0.1854 | 0.2623 | −0.0365 | 0.0058 | 0.0555 | −0.2224 | −0.2352 | −0.0704 |
| HES6 | −0.0119 | 0.0183 | −0.0092 | 0.0177 | −0.0254 | −0.0627 | −0.0277 | 0.0134 |
| TSTA3 | −0.1172 | 0.1692 | −0.1069 | 0.0687 | −0.1194 | 0.0914 | −0.0723 | 0.0857 |
| BTN3A2 | −0.0711 | −0.3017 | −0.0721 | −0.0114 | 0.0579 | −0.1565 | −0.3448 | 0.1660 |
| AK2 | −0.2401 | 0.0395 | −0.0265 | −0.0059 | 0.0826 | 0.2065 | −0.1449 | 0.2522 |
| ZNF292 | −0.1801 | 0.5430 | −0.0695 | 0.0387 | −0.2434 | −0.3219 | 0.1568 | −0.2005 |
| PTPN3 | −0.2565 | 0.2659 | −0.1155 | 0.0671 | −0.1907 | −0.1861 | −0.1891 | 0.2295 |
| MSH3 | −0.0586 | 0.2771 | −0.1240 | −0.0451 | −0.0601 | −0.2730 | −0.0208 | 0.0191 |
| EFCAB10 | 0.0340 | 0.1226 | 0.0173 | −0.0589 | −0.2638 | 0.0000 | −0.0204 | −0.0141 |
| PSMD1 | −0.0970 | 0.2732 | −0.0633 | 0.0339 | −0.1606 | −0.2464 | −0.2835 | −0.1587 |
| GPR162 | −0.0408 | −0.0272 | −0.0633 | 0.0454 | −0.5375 | −0.0159 | −0.2431 | −0.0071 |
| ANAPC5 | −0.3556 | −0.1716 | −0.1492 | −0.0656 | −0.2310 | −0.3569 | −0.0226 | 0.1333 |
| PIP | −0.0917 | −0.0142 | −0.0184 | −0.0180 | −0.0189 | 0.1646 | 0.1752 | −0.0141 |
| DENND2C | −0.2728 | −0.0299 | −0.0365 | 0.0172 | −0.1419 | −0.2332 | 0.0167 | 0.0249 |
| INTS3 | −0.2777 | −0.0153 | 0.0436 | 0.0995 | −0.1698 | −0.2095 | −0.1158 | −0.0539 |
| NUP50 | −0.1054 | −0.0272 | −0.0525 | −0.0396 | −0.1649 | −0.0251 | −0.1847 | −0.0283 |
| PPIL6 | −0.0475 | 0.3341 | −0.0352 | −0.1572 | −0.4874 | −0.0317 | −0.1033 | 0.0348 |
| TNFSF10 | −0.2180 | 0.2434 | 0.1291 | −0.0163 | 0.0807 | −0.0460 | −0.0788 | 0.3436 |
| RPS4X | −0.2226 | 0.0395 | −0.0533 | 0.2728 | −0.2405 | −0.0915 | −0.2059 | 0.3377 |
| SUN1 | −0.3133 | 0.2391 | −0.0780 | 0.1016 | −0.3493 | 0.1520 | 0.0808 | 0.2232 |
| PSMB6 | −0.1106 | 0.1559 | −0.1114 | 0.0454 | −0.0338 | 0.1125 | −0.1765 | −0.2515 |
| TRIM24 | −0.0836 | 0.0252 | −0.0255 | 0.1226 | −0.1438 | −0.3099 | 0.0917 | −0.1004 |
| CAB39 | 0.0440 | 0.1654 | −0.1069 | 0.0000 | −0.0557 | 0.3774 | −0.1155 | 0.0938 |
| FAM111A | −0.1697 | 0.3354 | 0.0173 | 0.0396 | −0.0296 | −0.0412 | −0.0742 | −0.2167 |
| RHOV | −0.3470 | 0.0463 | −0.1155 | −0.0120 | −0.0564 | −0.1031 | −0.0540 | 0.0148 |
| SSR3 | −0.2111 | 0.1949 | −0.0610 | −0.0575 | −0.2767 | 0.0725 | −0.0116 | 0.1718 |
| HSD17B13 | −0.0258 | −0.0245 | −0.0633 | −0.0114 | −0.2663 | −0.0732 | −0.3998 | 0.0529 |
| HNRNPH3 | −0.2457 | 0.0292 | −0.0809 | −0.0291 | −0.0279 | −0.1220 | −0.1532 | −0.1043 |
| WDR78 | 0.0002 | 0.0210 | 0.0421 | −0.2959 | −0.6342 | −0.0317 | −0.2227 | −0.0141 |
| CDK2AP2 | −0.0904 | −0.0452 | −0.0633 | 0.0000 | −0.0505 | −0.0297 | −0.2821 | 0.1137 |
| CFB | −0.4156 | 0.3152 | −0.0589 | 0.0230 | 0.0616 | −0.0825 | −0.0624 | 0.1769 |
| MLEC | −0.2236 | −0.0153 | −0.1114 | 0.0215 | −0.0778 | −0.1031 | −0.1140 | 0.0825 |
| ATP5F1D | −0.1943 | 0.0252 | −0.0275 | −0.0180 | −0.0946 | −0.2086 | −0.1206 | −0.1077 |
| NARS | −0.1964 | 0.5832 | −0.1420 | −0.0249 | −0.2325 | −0.2546 | 0.3147 | 0.2303 |
| CCDC74A | −0.0133 | −0.0180 | 0.0794 | 0.0331 | −0.2359 | −0.0473 | −0.1605 | 0.0277 |
| KRI1 | −0.1724 | 0.1439 | 0.0179 | 0.0058 | −0.1739 | −0.1155 | 0.0084 | −0.0126 |
| OMG | 0.0272 | 0.4657 | 0.0572 | −0.1748 | −0.8097 | −0.0159 | −0.0117 | 0.0614 |
| GRAMD2B | 0.1815 | 0.2179 | −0.0525 | −0.0217 | −0.3898 | 0.0772 | −0.3004 | −0.0199 |
| MPV17L | −0.0969 | −0.0334 | −0.0610 | −0.1074 | −0.2078 | 0.0862 | −0.2492 | 0.3093 |
| CMPK1 | −0.1801 | 0.1332 | −0.1915 | 0.0755 | 0.0563 | 0.0614 | −0.0700 | 0.2349 |
| C1orf43 | −0.2268 | 0.1555 | −0.0525 | 0.0444 | 0.0002 | 0.1050 | 0.0362 | 0.0825 |
| RPS27L | −0.1357 | 0.3503 | −0.0931 | 0.0269 | −0.0143 | 0.1050 | 0.0078 | 0.2582 |
| SLC28A3 | 0.0130 | 0.2685 | −0.0275 | 0.0236 | 0.0768 | −0.3829 | −0.0459 | −0.0349 |
| NACA | −0.2262 | 0.2760 | −0.0473 | 0.0107 | −0.1551 | −0.5158 | 0.1371 | 0.3206 |
| LY6D | −0.2852 | −0.0277 | −0.0247 | −0.0180 | −0.0182 | 0.1605 | −0.0359 | −0.0067 |
| MRFAP1L1 | −0.2830 | 0.1439 | −0.0089 | −0.0058 | −0.0071 | −0.2479 | −0.1051 | 0.0173 |
| LCORL | −0.0171 | −0.1192 | −0.0365 | −0.0298 | −0.0364 | −0.1520 | −0.0734 | −0.0330 |
| NBEAL1 | 0.1102 | 0.2404 | −0.0948 | −0.0988 | −0.1419 | −0.5541 | −0.1340 | 0.0137 |
| GBP3 | −0.0782 | −0.3132 | −0.0695 | −0.0056 | 0.1437 | 0.0385 | −0.1550 | 0.0059 |
| AK9 | −0.1305 | 0.0736 | 0.0167 | 0.0963 | −0.0087 | −0.1832 | −0.2708 | 0.1858 |
| DPM2 | −0.3266 | −0.0290 | −0.0721 | −0.0120 | −0.0254 | −0.2086 | 0.0693 | −0.0265 |
| TMX3 | −0.0911 | 0.3783 | −0.0721 | −0.0348 | −0.1311 | −0.0780 | −0.0684 | −0.0680 |
| DNAI1 | 0.0674 | −0.1537 | −0.0365 | −0.0672 | −0.1353 | 0.0000 | −0.1123 | 0.0418 |
| LCOR | −0.1959 | 0.2959 | −0.1520 | 0.0424 | −0.1962 | −0.2026 | −0.2315 | 0.1231 |
| USP46 | −0.3217 | −0.1071 | −0.0455 | −0.0239 | −0.1547 | −0.0265 | −0.0055 | 0.0687 |
| PHIP | −0.2606 | 0.3058 | −0.0569 | −0.0746 | −0.1079 | −0.4785 | 0.0539 | 0.0666 |
| ANKS1A | −0.1856 | 0.3804 | −0.0455 | −0.0298 | −0.3397 | −0.1293 | 0.0349 | 0.0647 |
| SLC25A24 | −0.1097 | −0.0067 | −0.0544 | −0.0415 | 0.0574 | −0.1806 | −0.0722 | −0.0559 |
| NUDT3 | −0.1966 | −0.2235 | −0.0544 | −0.0647 | 0.2492 | −0.2479 | −0.1553 | 0.0970 |
| ST6GALNAC1 | −0.1442 | 0.5014 | −0.0569 | −0.0114 | −0.0449 | −0.2224 | −0.1961 | 0.1823 |
| BUB3 | −0.1261 | 0.1908 | −0.0544 | −0.0462 | −0.1438 | −0.2224 | 0.0266 | 0.0644 |
| SLC35A2 | −0.0586 | −0.1311 | −0.0184 | 0.0396 | −0.3097 | 0.0304 | 0.0667 | −0.0199 |
| DDX17 | −0.1052 | −0.0011 | −0.0073 | −0.1133 | −0.2101 | −0.3071 | −0.2801 | 0.2142 |
| WFDC6 | 0.0002 | −0.0826 | −0.0544 | 0.0282 | −0.3080 | −0.0159 | −0.2234 | −0.0071 |
| SSBP4 | −0.0342 | −0.0827 | −0.0721 | −0.0284 | 0.0761 | 0.0151 | −0.1920 | −0.0133 |
| TP53I11 | −0.2515 | 0.0673 | −0.0455 | −0.0120 | −0.1719 | −0.1565 | −0.0930 | −0.0395 |
| H3F3B | −0.1385 | 0.2836 | −0.1025 | 0.0318 | −0.3186 | −0.0395 | −0.1372 | 0.3326 |
| HINT1 | −0.2403 | 0.1439 | −0.2601 | 0.0355 | −0.0048 | 0.0647 | 0.1094 | 0.1166 |
| BTAF1 | −0.1353 | 0.5084 | 0.0348 | −0.0291 | 0.1799 | −0.2829 | 0.0404 | −0.1758 |
| TMEM123 | −0.2469 | 0.1046 | 0.0156 | −0.0493 | −0.0067 | −0.0690 | 0.0325 | 0.4010 |
| IFNAR1 | −0.2651 | 0.1565 | −0.2460 | −0.1155 | −0.0405 | 0.1699 | 0.0309 | 0.0215 |
| MED13L | 0.0895 | −0.3312 | −0.1069 | −0.0059 | 0.0153 | −0.0100 | 0.0569 | −0.0405 |
| CA12 | −0.3009 | −0.0826 | −0.0569 | −0.0059 | −0.1407 | 0.1203 | −0.0525 | 0.0489 |
| ZFYVE16 | −0.0050 | 0.1842 | −0.0896 | −0.0222 | 0.0148 | −0.3049 | −0.2032 | −0.0740 |
| ABO | −0.1284 | −0.0452 | −0.0184 | −0.0117 | 0.1422 | −0.1399 | −0.1723 | −0.0448 |
| CCT7 | −0.0192 | 0.0064 | −0.0695 | 0.0112 | 0.0333 | −0.0150 | −0.2008 | −0.0405 |
| ZSCAN31 | −0.1185 | −0.1192 | −0.0184 | 0.0177 | −0.1194 | −0.0931 | −0.2519 | −0.0406 |
| CACFD1 | −0.0510 | 0.1439 | −0.0184 | −0.0357 | −0.0169 | −0.0444 | −0.1323 | −0.0780 |
| NQO1 | −0.5508 | 0.5995 | −0.1081 | −0.3591 | −0.1708 | −0.0280 | 0.1491 | 0.4235 |
| FABP6 | −0.0136 | 0.3828 | 0.0089 | 0.0861 | −0.3347 | −0.0159 | −0.4541 | −0.0071 |
| ATF6 | −0.0481 | −0.0578 | −0.0089 | −0.0233 | −0.2599 | −0.0525 | 0.0701 | 0.1255 |
| GSTA2 | −0.0542 | −0.1077 | 0.1255 | 0.0168 | −0.6047 | 0.2630 | −0.1902 | −0.0071 |
| SUGT1 | 0.0286 | 0.0064 | −0.0439 | −0.0056 | −0.3153 | 0.0385 | −0.0911 | −0.1185 |
| RALBP1 | −0.3510 | −0.0106 | −0.0439 | 0.0000 | −0.2551 | −0.0109 | 0.0093 | 0.2630 |
| SYS1 | −0.2066 | −0.2712 | −0.0365 | −0.0405 | 0.0376 | −0.0297 | −0.0267 | 0.0713 |
| COMMD2 | −0.1283 | 0.1324 | −0.0633 | −0.0120 | −0.1197 | −0.0473 | −0.1115 | −0.0065 |
| SAA2 | −0.3901 | −0.1548 | 0.1338 | −0.1206 | −0.0632 | −0.0627 | −0.6437 | −0.1123 |
| MRPL49 | −0.0886 | 0.0549 | −0.0544 | 0.0177 | −0.1467 | 0.1806 | −0.1587 | 0.1321 |
| TEKT1 | 0.0605 | 0.2384 | −0.0339 | 0.0303 | −0.0034 | −0.0159 | −0.0617 | 0.0000 |
| PTPN13 | −0.1749 | 0.1439 | −0.1325 | −0.0405 | −0.2291 | −0.0211 | −0.2756 | 0.2869 |
| MRPL41 | −0.0295 | −0.0520 | −0.0352 | 0.0225 | −0.3535 | 0.0406 | −0.0634 | 0.2345 |
| CDC16 | −0.0134 | −0.2298 | −0.0525 | −0.0167 | −0.1463 | 0.1255 | −0.1049 | −0.1037 |
| LRRC43 | −0.0067 | −0.3095 | −0.0365 | 0.0000 | 0.0464 | −0.0159 | −0.1840 | 0.0348 |
| LUC7L3 | −0.3289 | 0.0735 | −0.1851 | 0.0317 | −0.1248 | 0.0946 | −0.0220 | −0.1430 |
| CTCF | −0.1678 | −0.0860 | −0.0610 | −0.0532 | −0.1496 | 0.1387 | 0.0800 | −0.1829 |
| CIRBP | −0.5664 | −0.1298 | −0.1837 | −0.0213 | −0.5126 | −0.1069 | 0.2061 | 0.1685 |
| EFCAB12 | −0.0272 | 0.1736 | −0.0365 | 0.0225 | −0.1436 | 0.0000 | −0.1223 | 0.0000 |
| SPR | −0.0819 | −0.0676 | −0.0092 | −0.0175 | −0.1698 | −0.0627 | −0.1998 | 0.0395 |
| ZNF664 | 0.0142 | −0.1825 | −0.0177 | −0.0055 | −0.4826 | −0.2730 | −0.0554 | 0.0960 |
| PHYKPL | −0.0886 | −0.2213 | −0.0265 | −0.0931 | −0.0454 | −0.1278 | −0.0325 | 0.0312 |
| HSPA9 | −0.2910 | 0.1110 | −0.1031 | −0.1725 | −0.2471 | 0.0995 | 0.0607 | 0.0602 |
| ANAPC13 | −0.1408 | −0.1192 | −0.0809 | −0.0117 | −0.1642 | −0.0444 | 0.0136 | 0.0660 |
| FYB2 | 0.0002 | −0.3707 | 0.0000 | −0.0239 | −0.1112 | −0.1699 | −0.1822 | 0.0270 |
| INSR | −0.1762 | −0.0441 | −0.1155 | −0.0284 | 0.0562 | −0.0133 | −0.2590 | −0.0906 |
| MFN2 | −0.0981 | 0.0236 | 0.0348 | −0.0175 | −0.0900 | −0.0297 | −0.0164 | −0.0919 |
| SRSF7 | −0.1955 | −0.1253 | 0.1907 | −0.0054 | −0.3515 | −0.0952 | 0.0066 | 0.0641 |
| DZIP1 | −0.0204 | 0.1130 | −0.0455 | −0.0348 | 0.1370 | −0.0473 | 0.0736 | −0.0071 |
| H2AFZ | −0.1898 | −0.0122 | −0.0753 | 0.0339 | −0.2296 | −0.1478 | −0.0169 | 0.3937 |
| PPA1 | −0.1878 | −0.0325 | −0.1114 | 0.0282 | −0.0751 | −0.1430 | 0.0348 | 0.3505 |
| EVI5 | −0.0963 | 0.1162 | −0.0525 | −0.0332 | 0.1156 | −0.3410 | 0.2481 | 0.0127 |
| RPL6 | 0.0438 | 0.0426 | 0.0772 | −0.0507 | 0.0082 | −0.1699 | −0.0946 | −0.0827 |
| PPP1CB | −0.1577 | 0.0309 | −0.2895 | 0.0740 | −0.2001 | −0.1605 | 0.1432 | 0.4579 |
| TSR3 | −0.1434 | 0.0673 | −0.0455 | −0.0405 | −0.2919 | −0.0297 | 0.0512 | 0.2689 |
| SPG21 | −0.3844 | −0.0860 | −0.0177 | −0.0589 | 0.1507 | −0.0958 | −0.1853 | −0.1099 |
| HMGB1 | 0.0002 | 0.2237 | −0.0317 | −0.0495 | −0.2613 | −0.0418 | −0.0261 | −0.1449 |
| PTPRT | 0.0067 | 0.2959 | 0.0179 | −0.1044 | −0.3574 | −0.0317 | −0.3469 | −0.0280 |
| ANKRD36 | −0.0596 | 0.1511 | 0.0089 | −0.0589 | 0.0555 | −0.1640 | 0.1493 | −0.0199 |
| THOC2 | −0.2524 | 0.0482 | −0.1575 | −0.0340 | 0.0322 | −0.4988 | −0.0641 | −0.0473 |
| GLYR1 | −0.1390 | 0.4008 | 0.0086 | −0.0888 | −0.2222 | −0.0375 | −0.1020 | 0.2167 |
| ARL3 | −0.0280 | 0.3689 | −0.1155 | −0.0750 | −0.1444 | −0.1375 | −0.0110 | 0.0477 |
| UBP1 | −0.0969 | 0.0946 | −0.0983 | 0.0058 | −0.1633 | −0.1806 | −0.0874 | −0.0385 |
| ATAD2 | −0.1313 | −0.0452 | 0.0704 | −0.0415 | 0.0376 | −0.4270 | −0.1676 | 0.0825 |
| AFTPH | −0.3172 | 0.4434 | −0.0525 | −0.0171 | −0.1091 | 0.0914 | −0.1988 | 0.0924 |
| ORMDL1 | −0.2543 | 0.2084 | −0.0896 | −0.0532 | −0.0883 | −0.1155 | −0.0725 | −0.0498 |
| MEA1 | −0.1081 | 0.0426 | 0.0000 | −0.0818 | −0.1816 | −0.0141 | −0.0449 | 0.3155 |
| SLC25A36 | 0.0306 | 0.2314 | −0.0995 | −0.1026 | 0.0448 | 0.1500 | 0.0233 | 0.1498 |
| RAET1E | −0.0408 | 0.1908 | 0.0000 | −0.0532 | −0.2747 | −0.0159 | −0.1709 | 0.0684 |
| DNAI2 | 0.0208 | −0.2010 | −0.0544 | 0.0000 | 0.0904 | 0.0000 | −0.0308 | −0.0071 |
| TEX26 | 0.0208 | 0.1308 | −0.0544 | −0.0507 | −0.4655 | 0.0000 | −0.2690 | 0.0000 |
| GTF2A2 | −0.2236 | 0.1179 | −0.0339 | −0.0228 | −0.0680 | 0.0431 | −0.1953 | −0.0356 |
| NIPAL3 | −0.2477 | 0.2005 | 0.0348 | −0.0239 | −0.3317 | 0.1571 | −0.1979 | 0.3755 |
| GFM2 | 0.0345 | 0.1804 | −0.0983 | 0.0220 | −0.0798 | −0.0931 | −0.1949 | 0.1959 |
| DIS3 | −0.0046 | 0.0236 | −0.0633 | −0.0233 | −0.1849 | −0.2345 | 0.0600 | −0.1540 |
| ERMP1 | −0.2686 | −0.0807 | −0.0455 | −0.0180 | −0.0778 | −0.0444 | −0.1872 | 0.0124 |
| EFHC2 | −0.0136 | 0.2466 | −0.0184 | 0.0112 | −0.7186 | 0.0000 | −0.1469 | 0.0348 |
| NME7 | 0.0253 | 0.2899 | −0.1031 | −0.1180 | 0.1289 | −0.0627 | −0.1423 | −0.1414 |
| ESRP1 | −0.1518 | 0.1775 | −0.0809 | −0.0931 | −0.1360 | −0.0126 | −0.2050 | 0.1061 |
| ADGRG1 | −0.0912 | −0.1408 | −0.0809 | −0.0291 | −0.2007 | −0.0251 | 0.1809 | 0.0390 |
| ST13 | 0.0196 | 0.2959 | −0.0423 | −0.1155 | 0.1652 | 0.2157 | −0.0833 | −0.0485 |
| SRGAP3 | −0.1856 | 0.2675 | −0.0695 | −0.0473 | −0.0243 | −0.3569 | −0.1382 | 0.0438 |
| C16orf71 | 0.0071 | 0.2845 | −0.0092 | −0.0647 | −0.2514 | 0.0000 | −0.2321 | −0.0211 |
| SPAG1 | −0.0608 | 0.5452 | −0.0409 | −0.0656 | −0.1804 | −0.1399 | −0.1316 | 0.1759 |
| HNRNPK | −0.1462 | 0.5640 | −0.0758 | −0.0811 | −0.2095 | 0.1476 | 0.2143 | 0.0088 |
| FAM104B | −0.0586 | −0.0245 | −0.0265 | −0.0688 | −0.1300 | −0.0159 | −0.1701 | −0.0487 |
| ABCA13 | −0.0882 | 0.4378 | 0.1110 | −0.0856 | −0.0118 | −0.3475 | −0.1805 | −0.0903 |
| CEP104 | −0.1924 | 0.2363 | −0.0275 | −0.0058 | −0.2872 | 0.1325 | −0.2977 | −0.0305 |
| LRRC34 | −0.0067 | 0.0284 | −0.0365 | −0.0462 | −0.1560 | 0.0000 | −0.1926 | 0.0000 |
| CCP110 | −0.0929 | 0.4459 | −0.0455 | −0.0055 | −0.4103 | 0.0127 | 0.0342 | −0.1541 |
| RBM19 | −0.1618 | 0.2331 | −0.0455 | −0.0059 | −0.0726 | −0.5025 | 0.0884 | 0.1287 |
| C8orf59 | 0.1127 | 0.1097 | −0.1325 | −0.0462 | −0.1544 | −0.1520 | −0.0123 | 0.0696 |
| NDUFB7 | −0.2628 | 0.1681 | −0.1312 | −0.0451 | 0.1156 | −0.1731 | 0.0540 | 0.0667 |
| EIF5 | −0.1839 | 0.3093 | 0.2475 | −0.1130 | −0.2512 | 0.1162 | 0.0529 | 0.1444 |
| ENKUR | 0.0674 | 0.6623 | −0.1031 | −0.3265 | −0.1761 | −0.0159 | −0.3409 | 0.0348 |
| CALM3 | −0.0531 | 0.1314 | −0.0525 | −0.0761 | −0.1886 | −0.1832 | −0.0073 | −0.2651 |
| ADNP | −0.0370 | −0.2010 | −0.0525 | −0.0340 | −0.3317 | −0.2102 | −0.0871 | 0.0525 |
| CDC5L | 0.1311 | 0.0664 | 0.0000 | −0.0799 | −0.0496 | −0.1758 | 0.1411 | 0.2086 |
| AC006064.4 | −0.0504 | −0.0142 | −0.0409 | −0.2128 | −0.0189 | −0.0159 | 0.0653 | −0.0406 |
| KRT23 | −0.2312 | 0.0352 | −0.1114 | −0.1430 | −0.0854 | −0.3410 | −0.0583 | 0.0117 |
| PRDX6 | −0.1582 | 0.2320 | −0.0948 | −0.1232 | −0.3611 | −0.1096 | 0.0420 | 0.4014 |
| KDM5C | −0.1245 | 0.3325 | −0.0809 | −0.0298 | −0.2285 | −0.3937 | 0.0229 | 0.1829 |
| GON7 | 0.0127 | 0.1565 | 0.1255 | −0.0868 | −0.5945 | −0.0473 | 0.1251 | 0.2358 |
| ZKSCAN1 | −0.1991 | 0.1704 | −0.0915 | −0.0463 | −0.2983 | −0.2587 | −0.2078 | 0.3724 |
| TDG | −0.2336 | −0.1689 | −0.0633 | −0.0589 | −0.0632 | −0.1375 | 0.0001 | 0.1125 |
| SLC25A5 | 0.0056 | 0.2714 | −0.1155 | −0.1343 | −0.2686 | −0.1832 | 0.1770 | 0.3119 |
| ZC2HC1A | −0.0441 | 0.2791 | −0.1155 | −0.1208 | −0.2432 | −0.1081 | 0.1587 | 0.0138 |
| SPAG16 | −0.0976 | 0.7890 | −0.0339 | −0.0651 | 0.0093 | −0.0444 | 0.1557 | 0.0065 |
| MMACHC | −0.0441 | 0.0328 | 0.0754 | −0.5784 | −0.0263 | 0.0614 | 0.0996 | −0.1520 |
| BEST1 | −0.0872 | −0.0676 | −0.1570 | −0.3335 | −0.1804 | −0.0317 | 0.0153 | 0.0329 |
| RNF19A | −0.0141 | 0.0165 | 0.0251 | −0.0875 | 0.0778 | −0.2419 | 0.0181 | 0.1019 |
| DYNC2H1 | −0.0933 | 0.1758 | 0.1468 | −0.2972 | −0.0393 | −0.2224 | −0.3663 | −0.0843 |
| CFAP36 | 0.0112 | 0.1883 | −0.0610 | −0.1977 | −0.3431 | −0.1015 | 0.0948 | −0.0589 |
| CLU | −0.2745 | 0.5464 | −0.1712 | −0.2951 | −0.0108 | −0.8281 | −0.2062 | 0.4381 |
| MT-ND4 | −0.0466 | −0.2652 | 0.3011 | 0.8197 | −0.5463 | −0.1527 | −0.0035 | 0.3913 |
| DUSP1 | 0.4712 | 0.1234 | −0.2157 | 0.5268 | −0.0131 | 0.6443 | −0.0806 | −0.2712 |
| AQP5 | −0.0048 | 0.1703 | −0.1185 | 0.1475 | 0.0881 | 0.3173 | −0.2489 | −0.1878 |
| RPL27 | −0.1373 | 0.5560 | −0.0802 | 0.2937 | −0.1600 | 0.0304 | 0.2699 | 0.1243 |
| DAAM1 | 0.0455 | 0.3341 | −0.0423 | 0.1312 | 0.2632 | 0.3857 | 0.0364 | −0.0111 |
| ATG3 | −0.0221 | −0.1064 | −0.0506 | 0.2332 | −0.0854 | 0.1890 | 0.1238 | 0.3286 |
| PKN2 | −0.0922 | 0.0932 | 0.1699 | 0.1029 | −0.1804 | 0.2065 | 0.0752 | 0.0103 |
| LCN2 | −0.8219 | 0.0352 | −0.0931 | 0.1549 | 0.2379 | 0.1864 | −0.6963 | 0.4512 |
| CCDC186 | −0.2240 | 0.4780 | 0.0358 | 0.1844 | 0.1785 | 0.1392 | 0.1597 | 0.4788 |
| KIF27 | 0.0769 | 0.3414 | 0.0436 | 0.1508 | 0.0174 | −0.0280 | −0.0532 | 0.1289 |
| NCL | −0.1895 | 0.3763 | −0.0589 | 0.3027 | 0.1980 | 0.0320 | 0.2672 | 0.3423 |
| ZNF141 | −0.0316 | −0.0961 | −0.0275 | 0.0644 | 0.2066 | 0.2494 | 0.0321 | 0.1858 |
| CFAP69 | 0.0204 | 0.4750 | 0.0270 | 0.0379 | 0.2141 | 0.1605 | −0.1142 | 0.0208 |
| BAZ1A | −0.0190 | −0.2600 | 0.0879 | 0.1755 | 0.2105 | 0.0544 | −0.0544 | 0.0468 |
| CD82 | 0.0706 | 0.1082 | 0.0083 | 0.1828 | 0.1659 | −0.0431 | 0.1392 | 0.2963 |
| TCEAL3 | 0.0322 | 0.0825 | −0.0275 | 0.1485 | −0.0883 | 0.0458 | 0.5363 | −0.0623 |
| ADGRF1 | −0.3693 | 0.1439 | −0.0307 | 0.1458 | 0.2080 | 0.1338 | −0.0506 | 0.0474 |
| PPARGC1A | −0.0064 | 0.0508 | −0.0455 | 0.0786 | 0.1257 | 0.1489 | 0.0584 | 0.0196 |
| BCAR1 | −0.0087 | 0.2320 | 0.0270 | 0.0584 | 0.3372 | 0.1255 | −0.0389 | 0.0289 |
| SERPINB1 | −0.0934 | 0.1928 | −0.1623 | 0.0317 | −0.2222 | 0.1790 | −0.0568 | 0.2824 |
| TES | 0.2095 | 0.3461 | 0.0324 | 0.1052 | 0.0537 | 0.0270 | 0.0843 | 0.3973 |
| SELENBP1 | −0.1486 | 0.4604 | −0.1915 | 0.2575 | 0.2131 | −0.0365 | 0.0746 | 0.3499 |
| CAPZA2 | −0.0372 | 0.0768 | −0.0086 | 0.1110 | 0.1446 | 0.2487 | 0.0529 | 0.1926 |
| PERP | 0.0484 | 0.7229 | −0.1006 | 0.1822 | −0.0296 | 0.0317 | 0.1844 | 0.4870 |
| MT-ND2 | −0.2341 | −0.0168 | 0.1859 | 0.6331 | 0.0076 | 0.0255 | 0.1154 | 0.5726 |
| CANX | −0.3265 | 0.0891 | −0.0245 | 0.1151 | −0.4945 | −0.1651 | 0.0908 | 0.6096 |
| MARVELD3 | 0.0733 | 0.3756 | −0.0184 | 0.0687 | 0.0491 | 0.0406 | −0.2258 | −0.0251 |
| LDLR | −0.0461 | 0.2904 | 0.0151 | 0.1061 | 0.1799 | 0.1414 | 0.2634 | 1.0777 |
| CAMLG | 0.1771 | 0.1314 | 0.0436 | 0.0000 | 0.1830 | 0.2783 | −0.0180 | 0.0124 |
| FASN | −0.0599 | −0.0290 | −0.0544 | 0.0296 | 0.1757 | 0.2065 | 0.0186 | 0.7846 |
| ANO10 | 0.1677 | 0.0899 | 0.0794 | 0.0236 | 0.0796 | 0.1489 | −0.0655 | 0.3626 |
| ARID4B | −0.1547 | 0.0064 | −0.1040 | 0.0468 | 0.2632 | 0.3132 | 0.2151 | 0.2244 |
| SLC26A2 | 0.0003 | 0.0267 | 0.0235 | 0.0804 | 0.4696 | 0.4036 | 0.2488 | 0.0662 |
| CNTRL | −0.0829 | 0.5605 | 0.0910 | 0.1255 | 0.0794 | 0.0914 | −0.1616 | 0.0733 |
| HECTD4 | −0.1912 | 0.4803 | 0.0942 | 0.0396 | 0.6477 | 0.2912 | 0.1207 | −0.1155 |
| MYL6 | −0.3685 | 0.8342 | 0.0614 | 0.3466 | −0.0202 | 0.2828 | 0.0119 | 0.0874 |
| GABRP | −0.0878 | −0.0122 | −0.1775 | 0.3370 | −0.0609 | −0.1851 | 0.1242 | −0.0687 |
| ATP5PD | −0.0193 | −0.0406 | 0.0740 | 0.1717 | −0.1540 | 0.0780 | 0.2753 | 0.0337 |
| PHB2 | 0.0616 | 0.3190 | 0.1120 | 0.0289 | 0.0371 | −0.0732 | 0.1022 | 0.2630 |
| ZFYVE21 | −0.1431 | 0.1804 | 0.0270 | 0.0745 | 0.0826 | 0.1890 | −0.0599 | 0.0946 |
| CD59 | 0.1137 | 0.5817 | 0.0075 | 0.1712 | 0.1168 | 0.2863 | −0.0288 | 0.3913 |
| CYTH1 | −0.0253 | 0.1565 | −0.0352 | 0.1029 | −0.1729 | −0.1155 | 0.0903 | 0.0712 |
| JMY | 0.0055 | 0.2120 | −0.0780 | 0.1110 | 0.1538 | 0.0946 | 0.0696 | −0.0448 |
| PEX16 | −0.0778 | 0.0637 | 0.1069 | 0.0296 | 0.0002 | −0.0931 | 0.1141 | 0.0802 |
| ID1 | −0.2679 | 0.0899 | 0.0260 | 0.0524 | 0.1112 | 0.5585 | −0.1159 | −0.0303 |
| COQ9 | 0.1146 | 0.3580 | 0.1120 | 0.0177 | 0.1151 | 0.0875 | 0.0110 | 0.0557 |
| HNRNPA1 | −0.1800 | 0.2899 | 0.0716 | 0.0225 | 0.0491 | −0.1031 | 0.2345 | −0.1283 |
| KIAA1468 | −0.0652 | 0.2048 | 0.0825 | 0.0000 | 0.3027 | −0.0227 | 0.0147 | 0.0060 |
| PLEKHM2 | −0.0105 | 0.3083 | −0.0184 | 0.0177 | 0.3221 | −0.1155 | 0.0108 | 0.0065 |
| CEP250 | −0.1418 | 0.1632 | 0.1392 | −0.0589 | −0.0659 | 0.1255 | 0.0083 | 0.0665 |
| ATP2B1 | −0.0761 | 0.3573 | 0.0679 | 0.1312 | −0.0570 | 0.0385 | 0.3956 | 0.0786 |
| NRBP1 | −0.1237 | 0.2434 | 0.0592 | 0.1284 | −0.0531 | 0.1125 | −0.0776 | 0.0671 |
| TNFRSF21 | 0.0083 | 0.0705 | 0.1301 | 0.0570 | −0.0557 | 0.1550 | −0.0725 | 0.0960 |
| ZNF226 | 0.0061 | 0.3647 | 0.0544 | 0.0628 | −0.0350 | −0.0875 | 0.1521 | 0.0385 |
| COX7B | −0.0903 | −0.0659 | 0.2322 | 0.0252 | 0.1173 | 0.0000 | −0.0515 | 0.3079 |
| HLA-E | −0.1583 | 0.4511 | −0.0065 | 0.1855 | −0.1634 | 0.2303 | −0.3160 | 0.7448 |
| NAA50 | −0.0813 | 0.0508 | 0.0679 | 0.0786 | −0.0158 | −0.1015 | 0.1951 | 0.0952 |
| TRMT44 | 0.0483 | 0.2179 | −0.0184 | 0.1018 | 0.2014 | 0.1890 | 0.0761 | 0.0597 |
| TNPO2 | 0.0692 | 0.1626 | 0.0089 | 0.0628 | −0.2441 | 0.4005 | −0.0250 | −0.0194 |
| TIMP2 | −0.1030 | −0.0142 | −0.0365 | 0.0177 | −0.0094 | −0.0444 | −0.0033 | 0.1719 |
| DDX60 | −0.0620 | 0.3432 | −0.0275 | 0.0570 | 0.3887 | 0.1543 | 0.1552 | 0.1352 |
| MLPH | −0.4828 | −0.0576 | 0.0436 | 0.1226 | 0.1103 | 0.0134 | −0.0058 | 0.1976 |
| RNF19B | −0.0944 | −0.0676 | −0.0184 | 0.0406 | −0.1238 | 0.2157 | 0.0088 | 0.2503 |
| BACE2 | −0.0902 | −0.0726 | 0.0854 | −0.0519 | 0.1040 | 0.1520 | 0.0797 | 0.1069 |
| ZBTB38 | −0.1732 | 0.0945 | 0.0336 | 0.1601 | 0.0236 | 0.1520 | 0.0874 | 0.3165 |
| ABCC1 | −0.0713 | 0.3306 | 0.1520 | 0.0000 | −0.0632 | 0.1747 | 0.2151 | 0.0438 |
| RNF213 | −0.2353 | 0.6365 | 0.2003 | 0.3297 | 0.2984 | 0.3169 | −0.0639 | 0.2665 |
| TSPAN15 | −0.0669 | 0.2005 | 0.0179 | −0.0120 | 0.3434 | 0.4898 | −0.0274 | 0.0115 |
| EPN2 | −0.1538 | 0.3432 | 0.0794 | 0.0225 | 0.2632 | 0.1699 | −0.0400 | 0.0946 |
| TJP3 | −0.3903 | 0.4590 | −0.0265 | 0.1116 | 0.1583 | 0.3081 | 0.0369 | 0.1685 |
| PCBP2 | −0.2469 | 0.2656 | −0.1468 | 0.0247 | 0.1044 | 0.6332 | 0.1835 | 0.2969 |
| CHCHD2 | −0.0558 | −0.0173 | −0.0423 | 0.0586 | −0.2283 | 0.2345 | 0.3397 | 0.1278 |
| AEBP2 | 0.0255 | 0.0443 | 0.0995 | 0.0230 | −0.0081 | 0.2762 | −0.0541 | 0.0825 |
| EIF3B | −0.1811 | 0.1153 | −0.0896 | 0.1369 | 0.1151 | 0.1897 | 0.0447 | 0.2406 |
| COMT | −0.0357 | 0.2148 | 0.0436 | 0.0524 | 0.1964 | −0.0280 | 0.0681 | 0.3460 |
| PUS1 | −0.0811 | 0.3878 | 0.0544 | 0.0983 | 0.0181 | −0.1229 | 0.1564 | 0.2291 |
| MXD1 | 0.1021 | −0.0005 | 0.1081 | 0.0000 | 0.1351 | −0.0150 | −0.0248 | 0.1829 |
| CSRNP1 | 0.2203 | −0.0005 | 0.0977 | 0.0524 | 0.0826 | 0.2479 | −0.0157 | 0.0956 |
| SENP6 | 0.2233 | 0.8100 | 0.0879 | −0.0114 | 0.0002 | 0.0566 | 0.1214 | 0.0211 |
| RACK1 | −0.1763 | 0.2257 | 0.0504 | −0.0265 | 0.0530 | 0.2285 | −0.0413 | 0.5225 |
| CLHC1 | −0.0400 | 0.1336 | 0.0361 | 0.0115 | −0.0084 | −0.0627 | 0.0214 | −0.0141 |
| HDAC9 | −0.0532 | 0.3333 | −0.0184 | 0.0687 | 0.0526 | 0.1178 | 0.0030 | 0.1222 |
| RNH1 | −0.3352 | 0.5198 | 0.0766 | 0.1401 | 0.0397 | 0.1193 | 0.1433 | 0.3543 |
| GDI2 | −0.0800 | 0.3259 | 0.1699 | 0.0641 | 0.1286 | −0.0690 | −0.1518 | 0.3833 |
| RHPN2 | 0.1629 | 0.0621 | −0.0455 | 0.0938 | 0.1364 | 0.1178 | 0.3198 | 0.0630 |
| IL13RA1 | 0.0002 | 0.4403 | 0.0251 | 0.1369 | −0.0369 | −0.2095 | 0.0351 | 0.3661 |
| SMAP2 | −0.1414 | 0.1912 | 0.0525 | −0.0171 | 0.1257 | 0.1747 | 0.0293 | 0.1959 |
| ZBTB4 | 0.0642 | 0.0797 | −0.0809 | 0.0745 | −0.0135 | −0.0280 | −0.0119 | 0.3542 |
| TPPP3 | 0.0814 | 0.4393 | 0.0595 | −0.0421 | −0.0727 | 0.3070 | −0.0315 | 0.0338 |
| SNAPC4 | −0.0628 | 0.8190 | −0.0365 | −0.0357 | 0.3372 | 0.1402 | 0.3122 | −0.0958 |
| CCDC173 | 0.0208 | 0.9127 | −0.0352 | 0.1967 | −0.1804 | −0.0159 | 0.3823 | 0.0000 |
| SAFB | −0.0170 | 0.5849 | 0.0942 | 0.0339 | −0.0063 | 0.2515 | −0.0575 | 0.1008 |
| HRASLS2 | −0.0054 | 0.5831 | −0.0184 | 0.0057 | 0.2096 | 0.0772 | −0.2117 | 0.5850 |
| GBP5 | 0.0328 | 0.0007 | 0.0270 | −0.0059 | 0.2084 | 0.0772 | 0.0829 | 0.0000 |
| SH3GLB1 | 0.1005 | 0.4324 | −0.0649 | 0.0995 | 0.1257 | 0.1543 | −0.0301 | 0.2157 |
| KHSRP | −0.0649 | 0.3208 | 0.0525 | 0.0000 | 0.0168 | −0.0297 | 0.0448 | 0.0885 |
| USP39 | −0.0149 | 0.2895 | 0.0614 | 0.0406 | −0.1029 | 0.1550 | 0.0360 | 0.1255 |
| ANKRD11 | −0.1853 | 0.8043 | 0.2385 | 0.0053 | 0.1952 | −0.2619 | 0.0159 | −0.1520 |
| CCNI | −0.1368 | 0.2397 | −0.0328 | 0.0220 | 0.2978 | 0.2883 | 0.0709 | 0.1671 |
| OTUD7B | 0.0659 | 0.3503 | 0.1301 | −0.0589 | 0.2257 | 0.0967 | 0.0001 | 0.1444 |
| IRF1 | 0.0692 | 0.6744 | −0.2730 | 0.0641 | 0.0002 | 0.2106 | 0.0055 | 0.1795 |
| CCDC6 | −0.0701 | 0.1776 | −0.0633 | 0.0230 | −0.0305 | 0.2065 | 0.1609 | 0.2281 |
| RAPGEFL1 | −0.0552 | −0.1689 | −0.0455 | 0.0177 | 0.1071 | 0.5585 | −0.0553 | 0.2881 |
| ZCRB1 | 0.0184 | 0.3783 | −0.0352 | −0.0340 | 0.1389 | −0.0280 | 0.0494 | 0.1142 |
| AUTS2 | 0.0368 | 0.3562 | −0.0864 | 0.0512 | −0.1568 | 0.1665 | 0.2610 | −0.0518 |
| SMIM5 | −0.0701 | −0.0578 | 0.1301 | 0.0745 | 0.0634 | 0.0614 | −0.0336 | 0.0802 |
| MAP1B | −0.0136 | 0.3249 | 0.1301 | −0.0462 | −0.1553 | 0.0772 | 0.5150 | −0.0071 |
| VRK2 | −0.0221 | 0.1555 | 0.0436 | 0.0844 | 0.0526 | 0.2330 | 0.2541 | 0.0584 |
| EIF6 | −0.3977 | 0.0001 | 0.1468 | 0.0289 | 0.2708 | 0.0431 | 0.0264 | 0.2292 |
| POLR2B | −0.2238 | 0.3756 | 0.0910 | 0.0444 | −0.1538 | 0.0740 | 0.2130 | −0.0120 |
| IFI16 | −0.2344 | 0.6159 | 0.0825 | 0.1365 | 0.1112 | 0.0161 | 0.2735 | 0.1646 |
| DOCK6 | 0.0554 | 0.0894 | −0.0455 | 0.0524 | 0.0796 | 0.3165 | 0.0726 | 0.1050 |
| TENT5A | −0.1427 | 0.2229 | −0.0265 | −0.0233 | −0.0163 | 0.5721 | −0.0122 | 0.4594 |
| DPM3 | −0.1854 | 0.5023 | 0.0000 | 0.1284 | −0.1518 | 0.1178 | 0.2598 | 0.3471 |
| BICDL1 | −0.1178 | 0.1328 | −0.0455 | 0.0177 | 0.2174 | 0.2382 | 0.0904 | 0.1402 |
| CEBPB | 0.0158 | 0.2053 | −0.0610 | −0.0462 | 0.0253 | 0.2182 | 0.0132 | 0.2345 |
| CRNN | 0.0401 | −0.0005 | −0.0092 | 0.0296 | −0.0284 | 0.0000 | −0.0139 | −0.0071 |
| CELSR1 | 0.0483 | 0.7414 | 0.1120 | 0.0331 | 0.0400 | −0.2338 | −0.0153 | 0.0696 |
| METAP2 | −0.0509 | 0.1218 | −0.0352 | 0.0917 | 0.0215 | 0.1812 | 0.0713 | 0.0439 |
| EPHA2 | −0.0716 | 0.1319 | −0.0089 | 0.2147 | 0.2947 | 0.2908 | 0.1311 | 0.1859 |
| FAM213A | −0.0497 | 0.7622 | 0.0592 | 0.0687 | 0.2005 | −0.1520 | 0.3429 | 0.2988 |
| S100A4 | −0.1329 | 0.1928 | 0.2996 | 0.0975 | 0.1435 | 0.4930 | 0.3305 | 0.2313 |
| MED15 | 0.0053 | 0.3411 | −0.0455 | 0.0804 | −0.0153 | 0.1255 | −0.0193 | 0.0629 |
| LRP8 | 0.1280 | 0.4552 | 0.0270 | 0.0584 | 0.2406 | 0.0931 | 0.1457 | 0.0138 |
| RPS15 | −0.0986 | 0.0304 | 0.0208 | 0.2096 | 0.1330 | −0.1096 | 0.2429 | 0.3737 |
| OAS3 | −0.1579 | 0.0586 | 0.0942 | 0.1401 | 0.1866 | −0.0860 | 0.3112 | 0.4611 |
| C2orf40 | 0.0208 | 0.3663 | 0.0260 | −0.0396 | 0.5212 | −0.0317 | 0.3366 | 0.0000 |
| KIAA1147 | 0.1844 | 0.2732 | 0.2065 | 0.0644 | −0.0245 | −0.0619 | −0.0575 | 0.1255 |
| TSPAN14 | 0.0117 | −0.0936 | 0.0942 | 0.0058 | −0.1633 | 0.1618 | −0.0269 | 0.1520 |
| HIVEP2 | 0.0233 | 0.4005 | 0.0270 | 0.0406 | 0.2201 | −0.1293 | 0.0449 | 0.0502 |
| ANXA5 | 0.0924 | 0.3696 | −0.0753 | 0.0053 | −0.0741 | 0.3141 | 0.0737 | 0.4768 |
| PLK2 | 0.1708 | −0.0005 | −0.0780 | 0.0115 | 0.1112 | 0.1747 | −0.0195 | 0.3795 |
| ZNF316 | 0.0121 | 0.3074 | −0.0092 | 0.0117 | 0.1257 | 0.0000 | 0.0788 | 0.0515 |
| TNNI3 | 0.0547 | 0.3008 | −0.0184 | −0.0180 | 0.4193 | 0.0000 | 0.4500 | −0.0141 |
| RABL2B | −0.0727 | 0.7836 | −0.0365 | 0.0225 | 0.2276 | −0.0317 | 0.1298 | 0.1325 |
| RBM17 | −0.1466 | 0.1529 | −0.0983 | 0.0172 | 0.1138 | −0.0653 | 0.1790 | 0.1543 |
| NTN1 | 0.0116 | 0.1032 | 0.0361 | 0.0347 | −0.0751 | −0.1015 | −0.0439 | 0.2858 |
| TNNI2 | −0.1868 | −0.2850 | −0.0365 | 0.0584 | 0.1541 | 0.2895 | 0.0187 | 0.2484 |
| CLASRP | −0.0771 | 0.2434 | 0.0179 | −0.0060 | 0.0181 | −0.0525 | 0.1391 | 0.1743 |
| YWHAE | −0.0806 | 0.6375 | 0.1079 | −0.0382 | −0.1859 | 0.2418 | 0.2728 | 0.2244 |
| GRM7 | −0.0067 | 0.5589 | 0.0452 | −0.0120 | −0.0338 | 0.0000 | 0.1781 | −0.0071 |
| ATP6V1G1 | −0.1927 | 0.0210 | 0.2630 | −0.0921 | 0.0002 | 0.2176 | 0.1019 | 0.3294 |
| EIF3E | −0.0218 | 0.1820 | −0.0896 | 0.0454 | 0.0085 | 0.0577 | 0.0542 | 0.1984 |
| CFH | −0.1658 | 0.2959 | 0.0536 | 0.0465 | 0.2226 | −0.3103 | −0.1406 | 0.2630 |
| ARRDC3 | −0.1912 | 0.5496 | 0.0336 | 0.0532 | −0.0407 | 0.4647 | 0.0957 | 0.0570 |
| CRYBG2 | −0.0126 | −0.0142 | −0.0275 | 0.0177 | −0.0378 | −0.0317 | 0.0035 | 0.1125 |
| PTP4A1 | 0.0769 | 0.0637 | −0.0238 | 0.2630 | 0.0236 | −0.0280 | 0.1544 | 0.1869 |
| TMPRSS11D | 0.0373 | −0.0005 | −0.0352 | −0.0180 | −0.0094 | 0.0286 | −0.0275 | 0.0845 |
| ZMIZ2 | 0.0934 | 0.3529 | 0.0452 | −0.0357 | 0.1138 | 0.1829 | 0.1319 | 0.0803 |
| SAR1B | −0.1404 | 0.0236 | 0.2082 | 0.0512 | 0.1872 | 0.2370 | −0.0226 | 0.2798 |
| TMEM231 | −0.0675 | 0.3655 | −0.0171 | 0.1520 | 0.1636 | −0.0780 | 0.2556 | −0.0280 |
| ARNTL2 | −0.0051 | −0.0578 | 0.0270 | 0.1195 | −0.0364 | 0.3774 | −0.0796 | 0.0825 |
| MYADM | 0.0187 | 0.0463 | 0.0910 | −0.0059 | 0.0438 | 0.1850 | −0.0115 | 0.1287 |
| TRAP1 | −0.1336 | 0.3849 | 0.0348 | −0.0291 | 0.0203 | 0.2259 | −0.0765 | 0.1390 |
| CHP1 | −0.0322 | −0.0578 | 0.0000 | 0.0164 | 0.4648 | −0.1565 | 0.0903 | 0.3954 |
| USP8 | −0.0639 | −0.0081 | −0.0409 | −0.0271 | 0.0329 | −0.1844 | −0.0351 | 0.1414 |
| HM13 | −0.0758 | 0.0352 | 0.0766 | 0.0112 | 0.0742 | 0.0867 | 0.0026 | 0.4832 |
| KPNA3 | 0.0485 | 0.5667 | −0.0352 | 0.1717 | 0.1337 | −0.0280 | 0.0959 | −0.0060 |
| SLC9A3R2 | 0.0306 | 0.2212 | 0.0885 | 0.0236 | −0.0087 | 0.0431 | −0.0119 | 0.3755 |
| IGF2BP2 | 0.1015 | 0.3383 | −0.0633 | −0.0233 | 0.0279 | 0.0684 | 0.0064 | 0.1493 |
| SLC37A1 | −0.1198 | 0.8157 | −0.0633 | 0.0786 | −0.1540 | −0.0589 | 0.0939 | 0.2387 |
| RPS24 | 0.1136 | 0.2694 | 0.0510 | 0.0269 | 0.0629 | 0.3251 | 0.2728 | 0.0773 |
| RASEF | 0.0723 | −0.0529 | 0.0436 | −0.0396 | −0.0071 | 0.2505 | −0.0622 | 0.0177 |
| SLC44A4 | −0.1069 | 0.6824 | −0.0533 | 0.2082 | 0.0972 | 0.4051 | 0.0030 | 0.2224 |
| IER3 | 0.2421 | 0.1439 | −0.1081 | 0.0347 | 0.1611 | 0.8771 | −0.0389 | −0.1602 |
| FOXA1 | 0.0414 | 0.5209 | 0.0679 | 0.0056 | −0.1932 | 0.3429 | 0.1049 | 0.2531 |
| PLCD3 | −0.0738 | −0.0005 | 0.0452 | −0.0060 | −0.0189 | 0.0772 | 0.0716 | 0.1190 |
| DEF8 | 0.1808 | 0.1001 | 0.0794 | −0.0239 | 0.1175 | 0.0286 | 0.0728 | 0.1321 |
| VCAN | −0.0067 | 0.4393 | 0.0270 | 0.0289 | −0.2458 | 0.1806 | 0.0208 | 0.0000 |
| 44450.0000 | 0.0489 | 0.1439 | 0.0173 | 0.0000 | 0.1339 | 0.1790 | 0.3101 | −0.0415 |
| STK36 | 0.1072 | 0.0489 | 0.0179 | 0.0230 | 0.0546 | 0.0875 | 0.0060 | 0.0462 |
| MINK1 | −0.0723 | 0.1546 | −0.0365 | 0.0347 | 0.1175 | 0.4220 | 0.2217 | 0.2566 |
| LMTK2 | −0.0048 | −0.0245 | −0.0633 | 0.1198 | −0.1849 | 0.0995 | 0.1194 | 0.2751 |
| GBF1 | −0.0895 | 0.1857 | 0.0348 | 0.0512 | 0.3907 | 0.3119 | 0.0187 | −0.0245 |
| POMT2 | −0.0727 | 0.6192 | 0.0704 | 0.0115 | 0.3804 | −0.0780 | 0.2826 | 0.1561 |
| MT-ATP8 | −0.0477 | 0.6982 | 0.0971 | 0.1220 | −0.0387 | 0.2685 | 0.4269 | 0.1056 |
| ZBED1 | −0.0059 | 0.0923 | −0.0184 | −0.0239 | 0.2237 | 0.0825 | 0.1389 | 0.1587 |
| MCU | −0.0797 | 0.0304 | −0.0177 | 0.0115 | 0.1220 | 0.4834 | 0.0306 | 0.3165 |
| SYT5 | 0.0208 | 0.6440 | −0.0092 | −0.0233 | 0.2214 | 0.0000 | 0.3991 | 0.0000 |
| ITGA6 | 0.0963 | 0.1439 | 0.0270 | −0.0473 | −0.0587 | 0.0431 | 0.1896 | 0.0060 |
| RSL1D1 | −0.0889 | 0.3207 | 0.1045 | −0.0228 | 0.2315 | 0.0385 | 0.2144 | 0.1330 |
| ABHD11 | −0.2348 | 0.3395 | −0.0089 | −0.0175 | −0.0778 | 0.0825 | 0.3070 | 0.2216 |
| BMPR1B | 0.0175 | 0.0304 | 0.1255 | 0.0296 | −0.2304 | 0.2330 | 0.1311 | 0.2693 |
| FGFBP1 | −0.0568 | −0.0005 | 0.0270 | −0.0060 | −0.0094 | 0.1850 | −0.0136 | 0.4486 |
| RASSF5 | −0.0448 | −0.0545 | 0.0704 | −0.0357 | −0.0808 | 0.2783 | 0.1400 | 0.0067 |
| TCN1 | 0.0893 | −0.0005 | 0.0270 | −0.0180 | 0.0002 | −0.0627 | 0.0478 | −0.0555 |
| SDC4 | −0.0247 | 0.3190 | 0.0614 | 0.1146 | −0.2444 | −0.2095 | 0.3397 | 0.1687 |
| SDCBP2 | −0.0040 | 0.4783 | −0.0089 | −0.0233 | −0.1719 | 0.2630 | 0.0860 | 0.2838 |
| PER3 | 0.0322 | 0.4420 | −0.0544 | 0.1853 | 0.3332 | 0.1125 | 0.1521 | 0.0067 |
| NUCKS1 | −0.2175 | 0.6953 | 0.0704 | −0.0050 | −0.0100 | 0.0458 | 0.1431 | 0.3602 |
| SLC16A9 | 0.0150 | 0.0768 | −0.0177 | −0.0415 | 0.0944 | 0.1890 | −0.1182 | 0.6504 |
| MRPS26 | −0.2362 | 0.4775 | 0.0270 | −0.0059 | 0.0826 | 0.0431 | 0.1374 | 0.1402 |
| IFIT3 | −0.1015 | 0.0637 | 0.1881 | 0.0117 | 0.1433 | 0.1255 | −0.0801 | 0.1532 |
| CHD1 | −0.1016 | 0.4360 | −0.1492 | 0.1543 | −0.0073 | 0.1092 | 0.0090 | 0.0107 |
| LRRC8A | −0.0953 | 0.0328 | 0.1301 | −0.0117 | 0.0094 | 0.3165 | 0.0063 | 0.2937 |
| HERC5 | 0.0917 | 0.5050 | −0.0092 | 0.0117 | −0.0273 | −0.1081 | 0.1149 | −0.0141 |
| SWAP70 | 0.1299 | 0.5131 | −0.0983 | 0.0057 | 0.2856 | 0.0110 | 0.3980 | 0.1854 |
| TMEM106B | −0.0358 | 0.0768 | 0.0506 | −0.0233 | −0.1621 | −0.0931 | 0.1993 | 0.0644 |
| YARS | −0.1273 | 0.2148 | 0.0089 | 0.0804 | −0.0315 | 0.2908 | −0.1114 | −0.0348 |
| N4BP1 | −0.0586 | 0.1776 | 0.0179 | 0.1343 | 0.1949 | 0.1618 | 0.2602 | 0.0276 |
| ALDH3A1 | −0.3791 | 0.6668 | 0.3426 | 0.4124 | −0.2516 | 0.6210 | 0.3294 | 0.4135 |
| TBC1D9B | −0.2311 | −0.0081 | 0.0614 | 0.0844 | 0.1811 | 0.1365 | 0.0176 | 0.3833 |
| TMPRSS2 | −0.0578 | 0.2859 | −0.0896 | −0.0348 | −0.2592 | 0.1890 | −0.1437 | 0.1956 |
| TRIP10 | −0.0504 | −0.0142 | 0.0089 | 0.0058 | 0.0887 | 0.2630 | 0.0678 | −0.0631 |
| MEPCE | 0.0233 | 0.0121 | −0.0275 | 0.0584 | 0.0085 | 0.3479 | 0.2034 | 0.0695 |
| AKAP13 | −0.0380 | −0.1126 | 0.2866 | 0.1006 | −0.3448 | −0.2439 | 0.2311 | 0.1423 |
| PEA15 | −0.0957 | 0.0508 | −0.0633 | 0.0117 | −0.0169 | 0.4227 | 0.0930 | 0.2130 |
| PELP1 | 0.1613 | 0.1681 | −0.0275 | −0.0059 | −0.0505 | 0.2345 | 0.1545 | 0.1375 |
| EAPP | −0.0701 | 0.0173 | 0.0592 | −0.0704 | 0.0446 | 0.0000 | 0.3316 | 0.0704 |
| ITPR1 | −0.0532 | 0.2474 | −0.0092 | 0.0058 | 0.3975 | 0.1026 | 0.2084 | 0.0544 |
| DIO2 | 0.0537 | 0.0463 | 0.0000 | −0.0060 | 0.1112 | 0.3410 | 0.0766 | −0.0418 |
| LSM8 | 0.0446 | 0.1324 | 0.0679 | 0.0465 | 0.0706 | 0.0134 | 0.2074 | 0.0191 |
| IRS2 | −0.2144 | 0.1032 | −0.0171 | 0.0058 | −0.0632 | 0.4220 | 0.1748 | −0.1210 |
| FXYD3 | −0.3319 | 0.8399 | 0.0312 | 0.1666 | 0.1458 | 0.4603 | −0.0345 | 0.3294 |
| TM9SF2 | −0.1256 | 0.3351 | 0.0000 | 0.0415 | −0.2799 | 0.5454 | 0.0543 | 0.2881 |
| CAPN5 | −0.2210 | 0.3543 | 0.0361 | 0.0117 | −0.2709 | 0.4150 | 0.2030 | 0.1029 |
| LETM1 | −0.0979 | 0.0653 | 0.0525 | −0.0239 | −0.0895 | 0.0914 | 0.0706 | 0.2447 |
| DDX24 | 0.0338 | 0.6078 | −0.1278 | 0.1466 | 0.1056 | 0.2524 | 0.3690 | 0.3230 |
| RSBN1L | −0.0826 | 0.2865 | 0.0942 | 0.1110 | 0.1081 | 0.1301 | −0.1571 | 0.1026 |
| SUPT6H | −0.0468 | 0.2370 | 0.1031 | 0.0230 | 0.3602 | 0.3833 | 0.0659 | −0.1758 |
| PKP3 | 0.0002 | 0.1082 | −0.0275 | 0.0117 | 0.0262 | −0.0150 | 0.1122 | 0.1566 |
| RARRES2 | −0.1236 | −0.1932 | 0.0348 | 0.1077 | −0.0523 | −0.0159 | 0.2167 | 0.0348 |
| DUS1L | −0.1242 | 0.3759 | 0.0173 | 0.1195 | 0.0440 | −0.1520 | 0.1002 | 0.3729 |
| C9orf78 | −0.2810 | 0.5398 | 0.0421 | 0.0938 | 0.0521 | 0.1255 | 0.0388 | 0.4633 |
| MDM4 | −0.2286 | 0.4099 | 0.1816 | −0.0348 | 0.0904 | 0.0614 | 0.1077 | −0.0721 |
| EPC1 | −0.1257 | 0.3536 | −0.0255 | 0.1970 | 0.3314 | 0.3119 | 0.1213 | 0.3789 |
| HNRNPA2B1 | −0.0731 | 0.3614 | 0.3149 | 0.0504 | −0.0867 | 0.0624 | 0.0473 | 0.7125 |
| CTSS | −0.1305 | 0.5881 | 0.0156 | −0.0520 | 0.0435 | 0.3196 | −0.0693 | 0.3703 |
| MROH1 | 0.0259 | 0.7777 | 0.0794 | −0.0175 | −0.1389 | 0.0914 | 0.1767 | −0.0126 |
| IL6R | −0.0133 | 0.6157 | −0.0092 | −0.0239 | −0.0609 | 0.7155 | 0.1547 | 0.0475 |
| CGN | −0.0117 | 0.3818 | 0.0000 | −0.0964 | −0.0084 | 0.6171 | −0.1507 | 0.2773 |
| SQSTM1 | −0.0524 | 0.6032 | −0.0371 | 0.0577 | 0.1336 | 0.2540 | 0.3231 | 0.1965 |
| SLC39A7 | 0.0348 | 0.1314 | −0.0809 | 0.0177 | −0.1818 | 0.3671 | 0.0320 | 0.4517 |
| GNAS | −0.2464 | 0.6881 | −0.1375 | 0.1850 | −0.0300 | −0.1193 | 0.0768 | 0.6821 |
| GPRC5A | −0.3248 | 0.0598 | −0.0806 | −0.1155 | 0.0002 | 0.5305 | −0.0065 | 0.1575 |
| FUS | −0.3125 | 0.4658 | 0.2157 | 0.1498 | 0.3050 | −0.0511 | 0.0523 | 0.0075 |
| CCDC66 | 0.0230 | 0.4508 | 0.0173 | 0.0600 | −0.1320 | −0.2630 | 0.2554 | 0.0385 |
| RAB14 | −0.1116 | 0.1314 | −0.0352 | −0.0291 | −0.0709 | 0.4874 | −0.0112 | 0.3748 |
| ACTB | 0.0285 | 0.1546 | −0.0133 | −0.0309 | −0.1348 | 0.2751 | 0.2809 | 0.6563 |
| FAM219B | −0.1997 | 0.5287 | −0.0633 | −0.0704 | 0.4463 | 0.2895 | 0.1486 | 0.0427 |
| DUOX1 | −0.0436 | 0.2737 | 0.0614 | 0.0824 | −0.0611 | −0.0506 | 0.1597 | 0.1758 |
| DNAJC3 | −0.1501 | 0.0705 | −0.0165 | 0.1593 | −0.2278 | −0.2102 | 0.2038 | 0.5126 |
| IRF2BPL | 0.0106 | 0.1681 | 0.0794 | 0.0058 | 0.2014 | 0.2487 | 0.1256 | 0.2188 |
| ADIRF | −0.0696 | −0.0412 | 0.0173 | 0.0117 | −0.0839 | 0.1605 | 0.0590 | −0.0492 |
| SLC7A11 | −0.1727 | 0.3023 | −0.0544 | −0.0180 | 0.2159 | 0.1646 | 0.2888 | 0.0338 |
| ACBD3 | −0.1576 | −0.1311 | −0.0086 | −0.0114 | 0.2066 | −0.1444 | −0.1126 | 0.3643 |
| CBX3 | −0.1000 | 0.3465 | 0.0825 | −0.0780 | 0.1551 | 0.2515 | 0.1340 | 0.2201 |
| CD24 | −0.2690 | 0.3563 | −0.2515 | 0.0952 | −0.0939 | 0.1325 | 0.3997 | 0.4064 |
| FAF2 | −0.1533 | 0.4918 | 0.0089 | 0.1168 | −0.0471 | 0.1926 | −0.1797 | 0.1912 |
| DDIT4 | 0.1151 | 0.6409 | 0.0592 | 0.1699 | 0.1969 | 0.2908 | 0.0996 | 0.1380 |
| IQCB1 | 0.0409 | 0.2577 | 0.0260 | 0.0000 | −0.1453 | −0.0525 | −0.0894 | 0.0312 |
| SNX9 | −0.1342 | 0.3543 | −0.0275 | −0.0473 | 0.0337 | 0.3093 | 0.0339 | −0.0843 |
| ST8SIA4 | −0.0400 | 0.3878 | 0.0452 | −0.0532 | 0.1103 | 0.0304 | 0.1145 | 0.1790 |
| MEF2A | 0.0104 | −0.1736 | 0.1298 | −0.0114 | 0.1799 | 0.4220 | 0.1161 | 0.0191 |
| WDR90 | 0.0311 | 0.7863 | −0.0721 | 0.0168 | −0.0139 | 0.0431 | 0.3439 | 0.0802 |
| RIOK3 | 0.0368 | 0.2904 | 0.0324 | 0.1508 | 0.0826 | 0.3119 | −0.0942 | 0.2580 |
| PPARGC1B | 0.2518 | 0.0797 | −0.0184 | −0.0120 | −0.0564 | 0.5564 | 0.0190 | −0.1979 |
| RPS13 | 0.0002 | 0.4334 | 0.1038 | 0.0703 | −0.0151 | 0.0000 | 0.1231 | 0.1476 |
| CBX6 | −0.1373 | 0.0923 | 0.0704 | −0.0357 | 0.2214 | −0.0444 | 0.0322 | 0.3479 |
| MTRNR2L6 | −0.0714 | 0.3238 | 0.2214 | 0.0324 | 0.1522 | 0.0489 | 0.3367 | −0.1484 |
| GALNT12 | −0.1697 | 0.2845 | −0.0265 | 0.0570 | −0.1373 | 0.2091 | 0.1569 | 0.3931 |
| NARF | −0.1423 | 0.6001 | 0.1031 | −0.0175 | 0.2331 | 0.1790 | 0.2351 | 0.1409 |
| PALLD | −0.1616 | 0.9034 | −0.1575 | 0.1200 | 0.1337 | −0.1293 | 0.2327 | 0.2949 |
| RPS15A | −0.1495 | 0.3718 | 0.0577 | −0.0053 | −0.0456 | −0.0395 | 0.0347 | 0.2003 |
| EMP1 | −0.0048 | −0.0412 | 0.0592 | −0.0704 | 0.0472 | −0.1081 | −0.0238 | 0.5368 |
| SMAD4 | 0.1367 | 0.4049 | 0.0089 | 0.0339 | −0.1276 | 0.1050 | −0.0213 | 0.0489 |
| SELENOF | 0.0152 | 0.3849 | −0.0171 | −0.0888 | −0.0173 | 0.0875 | 0.1151 | 0.3440 |
| PSME3 | −0.0227 | 0.3189 | −0.0275 | −0.0405 | 0.2477 | −0.1081 | 0.0954 | 0.3747 |
| TMEM160 | −0.3027 | 0.4005 | 0.0592 | 0.0339 | 0.1339 | −0.0251 | 0.1002 | 0.1775 |
| UGCG | −0.1888 | 0.2320 | −0.2153 | 0.0917 | −0.0124 | 0.0231 | 0.2362 | 0.3610 |
| ZNF397 | −0.0393 | −0.1291 | 0.0270 | 0.0117 | 0.2708 | −0.0280 | 0.2264 | 0.0630 |
| FAM177A1 | −0.0291 | 0.0395 | 0.1255 | −0.0818 | 0.0077 | 0.3049 | 0.1968 | 0.1038 |
| SLU7 | 0.0175 | 0.5215 | 0.0083 | −0.0875 | −0.1084 | 0.0825 | 0.2697 | 0.1255 |
| RPL27A | −0.2447 | 0.9214 | 0.0908 | −0.0664 | −0.0907 | 0.1410 | 0.1471 | 0.2758 |
| RHOC | −0.2112 | −0.1318 | 0.1555 | 0.0786 | 0.0475 | −0.2630 | 0.1836 | 0.1417 |
| UBE2E1 | 0.1041 | 0.2306 | −0.0265 | −0.0291 | 0.0826 | 0.2479 | −0.0886 | 0.2065 |
| RAB5IF | −0.0667 | 0.0797 | 0.1255 | −0.0357 | −0.0169 | 0.1464 | 0.1786 | 0.1752 |
| MYOF | 0.1736 | 0.2278 | 0.0519 | 0.0641 | 0.0586 | 0.1912 | 0.1886 | 0.0645 |
| RPS4Y1 | 0.0002 | 0.7830 | −0.0255 | 0.0000 | 0.3027 | 0.0931 | 0.5339 | 0.1178 |
| TUBA1C | −0.2417 | 0.1820 | 0.1168 | 0.0172 | −0.0350 | 0.1605 | 0.1487 | 0.3993 |
| RAB9A | 0.0283 | 0.0768 | −0.0365 | 0.0745 | −0.1323 | 0.0875 | 0.1493 | 0.0262 |
| OGT | 0.0576 | 0.0570 | 0.0179 | −0.0058 | −0.0805 | 0.2895 | −0.0488 | 0.1660 |
| EIF5A | −0.0077 | 0.1308 | 0.0086 | 0.0056 | −0.0135 | 0.0286 | 0.0945 | 0.3789 |
| ZBTB7A | −0.3753 | 0.3809 | 0.0260 | 0.1312 | −0.1843 | −0.3219 | −0.0201 | 0.3696 |
| GNB2 | −0.0693 | 0.3424 | −0.0455 | 0.0057 | −0.0984 | 0.2182 | 0.1907 | 0.0198 |
| TNFRSF12A | 0.0401 | 0.0463 | −0.0184 | −0.0415 | 0.0094 | 0.0772 | 0.0246 | 0.1168 |
| PHF3 | 0.1432 | 0.3589 | 0.0078 | −0.0056 | −0.1916 | −0.0159 | −0.0770 | 0.1649 |
| PDZD8 | 0.2847 | 0.5147 | −0.0177 | −0.0761 | 0.1563 | −0.0375 | 0.1262 | 0.2878 |
| OAS1 | −0.2367 | 0.3138 | 0.0000 | 0.0236 | 0.1556 | 0.1444 | 0.1555 | 0.2179 |
| SPRR2D | 0.0340 | −0.0005 | 0.0000 | −0.0060 | 0.0002 | 0.0772 | −0.0243 | 0.0418 |
| RPS21 | −0.2026 | 0.7002 | −0.2955 | 0.0911 | 0.1362 | 0.1550 | 0.1819 | 0.4250 |
| TYMP | −0.2916 | 0.6139 | 0.0393 | 0.0517 | 0.2017 | 0.3626 | 0.1141 | 0.2579 |
| MPC1 | −0.2350 | 0.3727 | 0.0086 | 0.0500 | −0.0348 | −0.1806 | 0.0440 | 0.7139 |
| SLC4A1AP | 0.0560 | −0.0634 | −0.0983 | 0.0396 | −0.0153 | 0.3862 | 0.2097 | 0.1507 |
| EPS8L2 | −0.1713 | 0.3233 | −0.0177 | 0.1255 | 0.1051 | −0.0310 | 0.2441 | 0.1214 |
| PPP4R2 | 0.0002 | 0.3259 | −0.0265 | 0.0396 | 0.2710 | 0.1959 | 0.0741 | 0.0057 |
| TOP1 | −0.0752 | 0.2048 | −0.1114 | 0.1643 | 0.0985 | −0.0841 | 0.0745 | 0.1983 |
| MAN2C1 | −0.0701 | 0.7288 | 0.0977 | 0.0289 | −0.1323 | 0.3370 | 0.2911 | −0.0194 |
| KLK13 | −0.0639 | −0.0005 | −0.0184 | 0.0356 | 0.0002 | 0.0931 | 0.0001 | 0.1464 |
| UBR4 | −0.3416 | 0.5249 | 0.0348 | 0.1310 | 0.0511 | 0.2157 | 0.1563 | −0.0726 |
| CABIN1 | −0.0113 | 0.4484 | 0.0525 | 0.0570 | 0.2632 | 0.2768 | 0.2533 | 0.1193 |
| UBE3A | −0.1305 | 0.3833 | 0.0825 | 0.0396 | 0.2027 | 0.0637 | 0.1726 | 0.2834 |
| CCPG1 | −0.1547 | 0.5192 | 0.0980 | 0.0807 | −0.1973 | 0.2630 | 0.2527 | 0.0348 |
| TMBIM1 | −0.0120 | 0.0195 | −0.0544 | 0.0117 | 0.1818 | 0.0431 | 0.2112 | 0.2401 |
| RPL36 | −0.2758 | 0.0379 | −0.0902 | −0.0340 | 0.1219 | 0.0946 | 0.3035 | 0.5070 |
| COX6B1 | −0.2208 | 0.2397 | 0.0000 | 0.1784 | −0.0471 | 0.2746 | 0.2365 | 0.2729 |
| CLOCK | −0.1000 | 0.1908 | −0.0265 | −0.0175 | −0.1038 | 0.0684 | 0.1276 | 0.0924 |
| DDX60L | 0.0345 | 0.2474 | 0.1520 | 0.0584 | 0.0681 | 0.3755 | −0.0697 | −0.0321 |
| SEL1L | 0.0787 | 0.0304 | 0.0656 | 0.0671 | 0.0727 | −0.1015 | 0.0377 | 0.2751 |
| UNC93B1 | −0.2347 | 0.4180 | −0.0809 | 0.0861 | 0.1500 | 0.3119 | 0.2134 | 0.2106 |
| KRT18 | −0.1777 | −0.0867 | −0.1699 | 0.0911 | 0.0165 | 0.1841 | −0.1702 | 0.6886 |
| MTDH | −0.0096 | 0.4434 | −0.0753 | 0.0489 | −0.2222 | 0.0361 | 0.0864 | 0.3359 |
| TXNIP | −0.2670 | 0.2029 | −0.1735 | 0.2681 | 0.6942 | 0.1255 | −0.2492 | 0.6755 |
| F2RL1 | −0.0872 | −0.0142 | −0.0184 | −0.0120 | −0.0720 | 0.0931 | 0.1506 | 0.3272 |
| ARHGDIA | −0.0431 | 0.1051 | −0.0184 | 0.0804 | 0.0158 | 0.0825 | 0.2883 | 0.1778 |
| MT2A | −0.1886 | 0.0825 | 0.0000 | −0.0451 | −0.0169 | −0.1699 | 0.2130 | 0.6632 |
| EBP | −0.2121 | 0.4658 | −0.0365 | −0.0357 | 0.1730 | 0.3070 | 0.1780 | 0.4150 |
| CIR1 | 0.0742 | 0.5043 | −0.0525 | −0.0909 | −0.2274 | 0.0115 | 0.2011 | 0.1375 |
| CLK2 | 0.1183 | 0.5053 | 0.0977 | −0.0298 | 0.0253 | 0.1959 | 0.0516 | 0.0970 |
| KLF4 | 0.3756 | 0.3503 | −0.0962 | 0.1312 | 0.0658 | 0.6181 | 0.0044 | 0.1184 |
| AQP3 | 0.2318 | 0.8648 | −0.0978 | 0.2762 | −0.1187 | 0.1357 | 0.1496 | 0.2307 |
| FDPS | −0.0393 | 0.4546 | 0.0506 | 0.0339 | −0.3291 | 0.1137 | 0.2100 | 0.3075 |
| KLF5 | −0.0844 | 0.2401 | 0.2108 | −0.0370 | −0.1902 | 0.2088 | 0.0038 | 0.4438 |
| CEACAM1 | −0.0221 | −0.0412 | 0.0525 | −0.0120 | 0.0472 | 0.0458 | 0.0035 | 0.0515 |
| PRKAR1A | 0.0256 | 0.5659 | 0.2265 | −0.0738 | −0.2223 | 0.1050 | 0.0202 | 0.2728 |
| SLK | −0.4750 | 0.2434 | −0.1864 | 0.0242 | −0.1276 | 0.3160 | 0.0894 | 0.3852 |
| DNAJC15 | 0.0670 | 0.3878 | 0.0679 | 0.0331 | 0.0002 | −0.0280 | 0.1018 | 0.3395 |
| SPATS2L | −0.2067 | 0.4809 | 0.0086 | −0.0614 | 0.0311 | 0.2630 | 0.0180 | 0.4193 |
| LPCAT4 | −0.1656 | 0.1688 | −0.0365 | 0.0524 | 0.1928 | 0.1414 | 0.1346 | 0.2539 |
| CHD2 | −0.1397 | −0.2222 | −0.0806 | 0.1310 | 0.4916 | −0.2150 | 0.2128 | 0.1480 |
| B3GNT5 | −0.1047 | 0.5543 | 0.1081 | −0.0051 | −0.1898 | 0.1026 | 0.2104 | 0.0684 |
| RABL6 | −0.3573 | 0.2607 | 0.0000 | 0.1601 | 0.0358 | 0.0557 | 0.1235 | 0.1231 |
| KTN1 | −0.2176 | 0.8052 | −0.0117 | −0.0948 | 0.3761 | 0.5921 | 0.0857 | 0.3738 |
| PSMD3 | −0.0767 | 0.2845 | −0.0455 | 0.0172 | −0.0381 | 0.1890 | 0.0422 | 0.2224 |
| CD63 | −0.0511 | 0.4938 | 0.3205 | 0.1329 | −0.3052 | 0.2065 | 0.3084 | 0.4935 |
| GORASP2 | −0.1460 | −0.1064 | 0.0525 | −0.0298 | 0.0091 | 0.0725 | 0.0139 | 0.2868 |
| PER2 | 0.0075 | 0.4407 | −0.0695 | 0.1983 | 0.1659 | 0.3392 | −0.1544 | 0.3720 |
| RPP38 | 0.0495 | 0.4324 | −0.0275 | 0.0282 | 0.1928 | 0.2157 | 0.2303 | 0.3322 |
| SYAP1 | −0.2221 | 0.4555 | −0.0255 | −0.0831 | −0.2180 | 0.6509 | 0.0296 | 0.3219 |
| FUBP1 | 0.0224 | 0.2461 | 0.1904 | 0.0112 | 0.1377 | −0.2701 | 0.2139 | −0.1129 |
| STARD10 | −0.2310 | 0.2237 | 0.0179 | 0.0000 | 0.1969 | 0.3175 | 0.1737 | 0.1699 |
| HES1 | −0.0438 | 0.5831 | 0.1149 | 0.1681 | 0.3063 | 0.3906 | 0.1745 | 0.3679 |
| MYCBP2 | −0.0667 | 0.1526 | −0.1155 | 0.2166 | 0.1895 | 0.0525 | 0.2596 | 0.2744 |
| FEM1A | −0.1330 | 0.0637 | 0.1210 | 0.0406 | 0.0546 | 0.0151 | 0.1919 | 0.3206 |
| EEA1 | 0.0162 | 0.6134 | −0.0247 | −0.0688 | 0.2554 | 0.1055 | 0.0577 | 0.1122 |
| RSRC2 | 0.0167 | 0.4724 | −0.1224 | 0.0520 | 0.1822 | 0.3755 | 0.1759 | 0.1105 |
| SLC12A6 | −0.0153 | −0.0578 | 0.0179 | 0.0282 | −0.0778 | 0.3119 | −0.1481 | 0.2003 |
| RPL37A | −0.2003 | 0.5345 | −0.0297 | 0.0048 | 0.0904 | 0.0242 | 0.3130 | 0.2526 |
| RBM3 | 0.0045 | 0.4088 | 0.0161 | −0.0167 | −0.1476 | −0.1783 | 0.3306 | −0.0500 |
| ABHD2 | −0.1425 | 0.4843 | −0.0409 | 0.1154 | −0.3439 | 0.4150 | −0.0088 | 0.3513 |
| PDCD11 | 0.1598 | 0.2341 | −0.0721 | 0.0728 | 0.2931 | 0.0647 | 0.0137 | 0.0000 |
| SPINT1 | −0.1866 | −0.2051 | −0.0455 | 0.1077 | 0.1522 | 0.2895 | 0.1052 | 0.4544 |
| TFCP2L1 | −0.1302 | 0.2913 | −0.0423 | −0.0058 | −0.0273 | 0.8698 | −0.1014 | 0.3910 |
| DNM2 | −0.0421 | 0.3403 | 0.0525 | 0.0000 | 0.1477 | −0.2464 | 0.2404 | 0.4389 |
| BSPRY | −0.1912 | 0.1190 | −0.0365 | −0.0059 | −0.1438 | 0.2065 | 0.1767 | 0.2511 |
| IFIT2 | −0.0857 | −0.0005 | −0.0695 | 0.0671 | −0.0929 | 0.0000 | −0.2682 | 0.0665 |
| PLSCR1 | 0.1865 | 0.1945 | 0.0910 | −0.0743 | 0.1184 | −0.0375 | −0.0778 | 0.4150 |
| DUSP5 | 0.0860 | −0.0005 | −0.0506 | −0.0761 | −0.0094 | 0.1550 | 0.0299 | 0.2962 |
| METTL7A | −0.1076 | 0.4743 | 0.2130 | 0.0710 | 0.0749 | 0.2392 | 0.5550 | 0.5276 |
| ALDH1A1 | −0.0842 | 0.6777 | 0.0793 | 0.4999 | −0.6284 | 0.2119 | 0.0896 | 0.3701 |
| TACC2 | −0.2609 | 0.1961 | 0.0854 | 0.0557 | −0.0837 | −0.1312 | −0.4464 | 0.2451 |
| BEST4 | 0.0137 | 0.3426 | 0.0452 | −0.0964 | 0.1952 | −0.0159 | 0.2060 | −0.0071 |
| MVP | −0.0719 | 0.4918 | 0.0766 | 0.2344 | 0.0491 | 0.2041 | 0.0992 | 0.3203 |
| B4GALT4 | −0.0156 | 0.0121 | −0.0184 | 0.0289 | 0.0315 | 0.4081 | 0.1358 | 0.0000 |
| SF3B2 | 0.0002 | 0.6192 | 0.0406 | 0.0601 | 0.0674 | −0.0745 | 0.2798 | 0.1983 |
| FOXJ1 | 0.0605 | 0.6025 | 0.0704 | 0.3064 | 0.2281 | −0.0317 | 0.0609 | −0.0071 |
| LGALS3BP | 0.0206 | 0.0966 | 0.0260 | 0.0057 | 0.0358 | 0.1427 | 0.1306 | 0.5368 |
| RPL11 | −0.1676 | 0.5182 | 0.0186 | −0.0483 | −0.2049 | 0.1368 | 0.1766 | 0.3653 |
| REEP3 | −0.0646 | 0.2694 | 0.0000 | 0.0960 | 0.1730 | −0.0375 | 0.0693 | 0.3152 |
| LY6E | −0.2014 | 0.2768 | −0.2147 | −0.0291 | −0.0456 | 0.1255 | 0.5169 | 0.3770 |
| ATP5MPL | −0.0830 | 0.2959 | 0.0235 | 0.1335 | −0.2486 | 0.0000 | 0.2159 | 0.1080 |
| PTBP3 | 0.0002 | 0.3678 | 0.0740 | −0.0694 | −0.1991 | 0.0647 | 0.0482 | 0.4893 |
| FOSL2 | −0.0310 | 0.1439 | 0.0436 | 0.0339 | 0.2632 | 0.0684 | 0.1122 | 0.1595 |
| RPL7L1 | −0.2221 | 0.1439 | 0.1343 | 0.0000 | −0.1004 | 0.2908 | 0.2565 | 0.1812 |
| RRAD | −0.0267 | 0.5360 | 0.0406 | 0.4820 | 0.4249 | 0.2157 | −0.0484 | 0.0138 |
| VSIG2 | −0.4638 | −0.1192 | 0.0885 | 0.0703 | 0.2539 | 0.4834 | 0.0402 | 0.2518 |
| PPP1R10 | 0.0742 | 0.3539 | −0.0352 | 0.0768 | 0.2632 | 0.1926 | −0.0639 | 0.0196 |
| DOCK5 | −0.0387 | −0.0093 | 0.1255 | −0.0059 | 0.0002 | 0.5241 | 0.0238 | 0.2630 |
| REEP5 | −0.2147 | 0.3461 | −0.0439 | 0.1828 | −0.0529 | −0.0653 | 0.0589 | 0.2074 |
| TAF7 | −0.1667 | 0.3954 | 0.2370 | −0.0277 | −0.0057 | 0.0000 | 0.1293 | 0.0986 |
| CIART | −0.0067 | 0.1692 | 0.0000 | 0.2326 | 0.0979 | 0.2630 | 0.2268 | −0.0071 |
| B2M | −0.2445 | 0.9619 | 0.2462 | −0.0291 | −0.2663 | 0.2937 | 0.0655 | 0.5516 |
| SPTSSA | −0.0322 | −0.0412 | 0.1609 | 0.0687 | −0.1153 | 0.0614 | 0.0001 | 0.5168 |
| DRC3 | 0.1628 | 0.6275 | −0.0177 | 0.0269 | 0.0216 | 0.1489 | 0.1827 | −0.0071 |
| VCP | −0.2830 | 0.5544 | 0.0772 | 0.1627 | −0.1930 | 0.5241 | 0.0489 | 0.1115 |
| MTUS1 | −0.2221 | 0.2248 | −0.1520 | 0.1052 | 0.1536 | −0.2310 | 0.0671 | 0.5949 |
| SNRNP200 | −0.3185 | 0.0264 | 0.0000 | 0.0973 | 0.3264 | 0.1901 | 0.0458 | −0.0050 |
| SERINC2 | −0.2928 | 0.2642 | −0.0455 | −0.0239 | 0.2188 | −0.0133 | 0.3061 | 0.1539 |
| RSAD2 | −0.0778 | 0.0945 | −0.0184 | 0.0177 | −0.1323 | 0.2783 | 0.0804 | 0.2134 |
| MAGI3 | −0.0489 | 0.3351 | −0.0265 | 0.0570 | −0.1357 | 0.0317 | 0.0971 | 0.1954 |
| LMNA | −0.0029 | −0.0802 | 0.1168 | 0.1200 | 0.1151 | 0.4606 | 0.1001 | 0.5276 |
| ISG15 | 0.0860 | 0.4122 | −0.0589 | 0.0396 | 0.2798 | 0.3340 | 0.2430 | 0.2261 |
| DNAJC21 | −0.1846 | 0.3333 | −0.0525 | −0.0059 | 0.1928 | 0.3497 | 0.0386 | 0.2240 |
| EFHD2 | −0.0342 | 0.1218 | −0.0352 | −0.0114 | −0.2809 | 0.3626 | 0.0548 | 0.3098 |
| OASL | 0.1027 | −0.0142 | 0.0000 | 0.0177 | −0.0189 | 0.0151 | −0.0589 | 0.2146 |
| AES | −0.3084 | 0.2688 | −0.1926 | −0.1256 | −0.1428 | −0.0641 | 0.3582 | 0.2759 |
| ARPC5 | −0.1311 | 0.3208 | −0.0589 | 0.0112 | 0.0088 | 0.0867 | 0.2090 | 0.4788 |
| PTP4A2 | −0.0316 | 0.0586 | 0.0227 | −0.0325 | 0.0531 | 0.5481 | 0.0722 | 0.4034 |
| CCDC39 | 0.0208 | 0.5913 | 0.1841 | −0.0577 | 0.1852 | −0.0317 | 0.3295 | 0.0000 |
| ERCC3 | 0.2097 | 0.2642 | 0.0000 | 0.1018 | 0.1776 | 0.0914 | 0.2459 | 0.1022 |
| MT-ND3 | −0.3629 | 0.9104 | 0.0065 | 0.5904 | −0.2092 | 0.4448 | 0.5070 | 0.3610 |
| SCEL | 0.0347 | 0.0463 | 0.0942 | 0.0117 | −0.0471 | 0.0614 | −0.0695 | 0.0270 |
| CAMK1D | 0.0291 | 0.4419 | 0.2263 | 0.0115 | 0.1928 | 0.0786 | 0.0245 | 0.5297 |
| CLDN7 | −0.0235 | 0.2857 | 0.0875 | −0.0051 | −0.0683 | 0.1618 | 0.3065 | 0.1463 |
| PITPNM1 | −0.1208 | 0.3007 | 0.0173 | −0.0171 | 0.0909 | 0.1178 | −0.0833 | 0.2447 |
| ZC3H15 | 0.0334 | 0.1210 | 0.0080 | −0.0575 | 0.0475 | 0.4695 | −0.0387 | 0.3968 |
| CCND1 | −0.1891 | 0.1662 | −0.0834 | −0.0056 | −0.0731 | 0.3833 | 0.1310 | 0.5529 |
| CDCP1 | −0.0354 | 0.5589 | 0.0179 | 0.0628 | −0.0553 | 0.5284 | 0.0289 | 0.0687 |
| FAM3B | −0.1363 | −0.0578 | 0.0794 | 0.0584 | 0.3434 | 0.1699 | 0.2536 | 0.2681 |
| ELK3 | 0.1780 | 0.3503 | 0.1728 | −0.0233 | 0.1804 | −0.0780 | 0.0386 | 0.2820 |
| SLPI | −0.1766 | 0.6554 | −0.0804 | 0.2219 | 0.0932 | −0.1723 | −0.4071 | 0.5458 |
| PRR14L | −0.0719 | 0.1354 | 0.0336 | −0.0647 | 0.2422 | 0.3312 | 0.2020 | 0.1137 |
| DSP | −0.1995 | 0.7067 | −0.1191 | 0.1356 | 0.4737 | 0.1088 | 0.2140 | −0.0164 |
| CLIP1 | −0.0741 | 0.7760 | 0.1058 | 0.3996 | 0.0605 | 0.1387 | 0.1241 | 0.0704 |
| ITPR3 | −0.2082 | 0.3954 | −0.0455 | 0.0172 | 0.1131 | 0.5122 | 0.1317 | 0.0169 |
| DDR1 | −0.1057 | 0.3222 | 0.0995 | 0.0600 | −0.2454 | 0.2630 | 0.0944 | 0.2230 |
| XRCC5 | −0.2097 | 0.5696 | −0.0231 | 0.0053 | −0.2393 | 0.1812 | 0.1951 | 0.5216 |
| B3GALT5 | 0.1684 | −0.0142 | 0.0794 | −0.0239 | −0.0094 | 0.2487 | 0.0698 | 0.0825 |
| GSR | −0.3653 | 0.1776 | −0.0255 | −0.0378 | −0.0678 | 0.3119 | 0.3112 | 0.2780 |
| CTGF | −0.0328 | 0.5819 | −0.0086 | −0.0413 | 0.1628 | 0.0931 | 0.6760 | −0.0137 |
| PGD | −0.2327 | 0.1963 | 0.0348 | −0.0451 | 0.0480 | 0.3034 | 0.4612 | 0.4271 |
| PCDH1 | −0.1439 | 0.5215 | 0.0614 | 0.0236 | 0.1807 | 0.2330 | −0.0174 | −0.0386 |
| A4GALT | −0.2668 | 0.1067 | −0.0544 | 0.0524 | −0.0981 | 0.1778 | 0.2129 | 0.1069 |
| MAP3K8 | 0.0827 | 0.1550 | 0.0173 | 0.2454 | 0.0326 | 0.3070 | 0.1906 | −0.1155 |
| EGR1 | 0.3188 | 0.1167 | −0.0690 | 0.3923 | 0.3372 | 0.1255 | −0.1763 | 0.5197 |
| ATP12A | −0.0929 | 0.4831 | −0.0177 | 0.0465 | 0.4676 | 0.1026 | 0.3412 | 0.5133 |
| TXN | −0.1851 | 0.4152 | −0.0893 | 0.1167 | 0.2084 | 0.1800 | 0.4321 | 0.3467 |
| WNK1 | −0.1427 | 0.1591 | 0.1174 | 0.1954 | 0.3779 | 0.1874 | −0.1743 | 0.2014 |
| C9orf24 | 0.2309 | 0.7524 | 0.1674 | 0.2072 | −0.2577 | 0.0151 | −0.0777 | −0.0069 |
| IQGAP2 | 0.2732 | 0.3503 | 0.2200 | 0.0220 | 0.0198 | 0.4265 | −0.2168 | 0.1901 |
| LIMA1 | −0.0740 | 0.3007 | −0.1081 | 0.1061 | −0.0271 | 0.2630 | −0.0163 | 0.1383 |
| PTGES2 | 0.1127 | 0.1324 | −0.0544 | −0.0180 | 0.0349 | 0.1402 | 0.1285 | 0.0630 |
| MPRIP | −0.0132 | 0.2744 | −0.0525 | 0.0000 | 0.3707 | 0.3923 | −0.0164 | 0.4095 |
| CCDC80 | −0.0886 | 0.9824 | −0.1822 | 0.2334 | 0.2212 | 0.4930 | −0.2738 | 0.2308 |
| HMGCS1 | −0.0524 | 0.0379 | 0.0167 | 0.0056 | −0.1074 | −0.2895 | 0.3466 | 0.8175 |
| TSPAN1 | −0.3126 | 1.2060 | 0.1963 | 0.0275 | 0.1474 | 0.2206 | 0.1115 | 0.3400 |
| SPEN | −0.0406 | 0.4871 | 0.2285 | −0.0109 | 0.2012 | −0.0813 | 0.2273 | 0.2786 |
| MRPL3 | −0.0049 | −0.0196 | 0.1031 | 0.0058 | 0.2033 | −0.0780 | −0.0235 | 0.1225 |
| IL1RN | −0.1797 | 0.0463 | −0.0780 | 0.0628 | 0.1637 | 0.3251 | −0.0034 | 0.2468 |
| PRRC2C | −0.1624 | 0.4428 | −0.1361 | 0.0924 | −0.0185 | 0.0115 | 0.0955 | 0.1033 |
| EWSR1 | −0.0097 | 0.3981 | −0.0165 | 0.0444 | 0.1662 | −0.1740 | −0.0153 | 0.3473 |
| PRPF8 | −0.0610 | 0.3557 | −0.0423 | 0.0824 | −0.3241 | 0.0614 | 0.2944 | 0.1029 |
| MUC4 | −0.6090 | 0.3162 | 0.1409 | −0.1658 | 0.3997 | 0.2051 | 0.2723 | 0.2195 |
| SRRM2 | −0.0843 | 0.3134 | 0.1291 | −0.0578 | −0.4118 | −0.3833 | 0.0457 | 0.2247 |
| EPS8L1 | −0.5735 | −0.1896 | −0.0589 | −0.0291 | 0.0305 | 0.1008 | −0.1694 | 0.2228 |
| SPINK5 | 0.4605 | −0.0005 | 0.0000 | 0.0644 | 0.0002 | −0.4389 | 0.0336 | −0.0906 |
| FRMD4B | −0.0631 | 0.1336 | −0.0265 | −0.0117 | 0.1589 | 0.2392 | 0.3977 | 0.4150 |
| SERPINB2 | 0.0720 | 0.0463 | −0.0177 | −0.0532 | 0.0732 | −0.0317 | 0.2326 | 0.0925 |
| UQCR11 | 0.0031 | 0.0825 | 0.3557 | 0.1479 | −0.0858 | 0.4017 | −0.0012 | 0.1476 |
| CREBBP | 0.2189 | 0.1990 | 0.1081 | 0.0225 | −0.0500 | 0.1031 | −0.0553 | 0.1043 |
| TUBB | −0.1514 | 0.1570 | 0.0348 | 0.0745 | 0.1083 | −0.0297 | 0.0331 | 0.7614 |
| TRAPPC9 | 0.0814 | 0.2870 | 0.0270 | 0.0570 | 0.3336 | −0.0690 | −0.0239 | 0.3020 |
| NAV2 | 0.0230 | 1.0225 | −0.0809 | 0.0687 | 0.4255 | −0.1349 | 0.2012 | 0.0385 |
| CTSD | −0.4446 | 0.3733 | 0.0065 | 0.2105 | 0.1398 | 0.2003 | 0.1892 | 0.4019 |
| TPT1 | −0.0945 | 0.4515 | 0.0906 | 0.4089 | −0.2152 | 0.2224 | −0.0586 | 0.3982 |
| ATP6V0B | −0.1809 | 0.0508 | 0.1841 | 0.0164 | 0.0122 | 0.3497 | −0.0250 | 0.2964 |
| TTC9 | −0.0401 | 0.3138 | 0.1298 | −0.0233 | 0.0358 | 0.1255 | 0.0667 | 0.3370 |
| CPSF1 | −0.0757 | 0.1336 | 0.0885 | −0.0239 | 0.1297 | 0.2091 | 0.1403 | 0.2274 |
| CES2 | −0.2978 | 0.4658 | 0.0089 | 0.0557 | 0.1163 | 0.0914 | 0.3009 | 0.2198 |
| KRT6A | −0.3650 | −0.0005 | 0.0506 | −0.0415 | −0.0094 | 0.3991 | 0.0645 | 0.8340 |
| SLC2A1 | 0.0601 | 0.1550 | 0.0270 | 0.1198 | −0.2015 | −0.0280 | 0.3972 | 0.3017 |
| GAK | −0.1882 | 1.1656 | 0.0173 | 0.0057 | 0.0983 | 0.4731 | 0.1536 | 0.2905 |
| TRIM16 | −0.3087 | 0.2179 | 0.0089 | −0.0117 | 0.2372 | 0.2783 | 0.0791 | 0.1874 |
| SGK1 | −0.2004 | 0.1945 | 0.0242 | 0.1283 | 0.3236 | 0.7645 | 0.0341 | 0.3677 |
| NCF2 | 0.0934 | −0.0844 | 0.0977 | 0.0454 | 0.1170 | −0.0444 | 0.2811 | 0.0802 |
| CPEB4 | −0.0457 | 0.4835 | 0.0489 | 0.1436 | 0.2199 | 0.0110 | 0.1852 | 0.1769 |
| MAP3K13 | 0.0602 | 0.3809 | −0.1409 | 0.0168 | −0.0907 | 0.2756 | 0.0682 | 0.1078 |
| COX8A | −0.1425 | −0.0367 | −0.0589 | 0.0973 | −0.0263 | 0.2630 | 0.3891 | 0.4286 |
| PPP6R2 | −0.1362 | 0.3365 | −0.0455 | −0.0059 | 0.2966 | 0.4150 | 0.1222 | 0.0127 |
| RBM33 | 0.0048 | 0.1615 | −0.0864 | 0.0960 | 0.4191 | 0.1255 | 0.0735 | 0.2267 |
| RHOA | −0.2941 | 0.4658 | 0.1597 | −0.1278 | −0.0818 | 0.0786 | 0.2424 | 0.3268 |
| PTMA | −0.0409 | 1.0747 | 0.1738 | −0.1046 | 0.1348 | 0.1218 | 0.5033 | 0.6766 |
| GOLGA3 | 0.1547 | 0.4658 | 0.0525 | 0.0057 | −0.0669 | 0.1459 | 0.2407 | −0.0900 |
| IGFBP3 | 0.0181 | −0.0142 | −0.0864 | −0.0647 | −0.1109 | 0.1444 | 0.2848 | 1.1759 |
| METTL5 | 0.0194 | 0.3503 | 0.0995 | 0.0786 | 0.3577 | −0.0875 | 0.1769 | 0.0173 |
| PRRG4 | 0.0248 | 0.0705 | 0.0270 | 0.0000 | 0.2695 | 0.1520 | 0.1617 | 0.2099 |
| TAGLN2 | −0.0441 | 0.6177 | −0.0834 | 0.1413 | 0.3882 | 0.4695 | 0.1716 | 0.2538 |
| CD200R1 | 0.2060 | 0.1703 | 0.0179 | −0.0239 | 0.0768 | 0.2630 | 0.0652 | −0.0069 |
| FAU | −0.0305 | 0.5460 | −0.2352 | −0.0525 | 0.0932 | 0.6859 | 0.2265 | 0.4139 |
| ERBB2 | 0.2187 | 0.2139 | 0.1255 | −0.0112 | −0.2456 | −0.0100 | 0.0741 | 0.0000 |
| DDX3Y | 0.0002 | 0.8283 | 0.0348 | 0.0728 | 0.3925 | 0.2313 | 0.3085 | −0.0780 |
| PIGR | −0.2376 | 0.5808 | −0.0834 | 0.3830 | −0.7708 | 0.4349 | −0.1323 | 0.8679 |
| CFD | −0.1231 | 0.1302 | −0.0092 | 0.0524 | −0.0378 | 0.2330 | 0.1043 | 0.2439 |
| NTS | 0.1756 | −0.0277 | 0.0525 | −0.0171 | 0.1974 | 0.6245 | −0.1451 | 0.4271 |
| CD99 | −0.0228 | 0.4534 | 0.0592 | −0.0964 | 0.0803 | 0.1699 | 0.2349 | 0.2955 |
| PITPNA | 0.0540 | 0.2732 | 0.1210 | 0.0000 | 0.0305 | 0.0134 | 0.2292 | 0.4302 |
| ASAH1 | −0.2285 | −0.1296 | −0.1234 | 0.2684 | 0.0056 | 0.0614 | 0.1266 | 0.5187 |
| C1orf116 | 0.0224 | 0.0899 | 0.1210 | −0.0175 | 0.0768 | −0.0280 | −0.0980 | 0.4150 |
| ATXN2 | 0.0427 | 0.4720 | 0.0179 | 0.0220 | 0.1105 | 0.5361 | 0.0873 | 0.0908 |
| NPEPPS | −0.3296 | 0.5116 | 0.0421 | −0.0340 | −0.0203 | 0.3857 | 0.0558 | 0.2356 |
| PPP6R3 | −0.1523 | 0.4152 | 0.1383 | 0.1853 | 0.0706 | 0.0000 | 0.1591 | 0.1069 |
| SFN | −0.0319 | 0.2563 | −0.0171 | 0.0000 | −0.0364 | 0.0825 | 0.1495 | 0.5343 |
| GALNT5 | −0.1140 | 0.0508 | 0.0000 | 0.1110 | −0.0471 | 0.2115 | 0.1645 | 0.2630 |
| HK1 | −0.1661 | 0.5543 | 0.0942 | 0.0628 | 0.3050 | −0.3163 | 0.2647 | 0.0154 |
| KRT8 | 0.1444 | 0.6495 | −0.1964 | 0.0291 | 0.1727 | 0.0532 | 0.1918 | 0.5710 |
| PARP14 | −0.4376 | 0.9602 | −0.1822 | 0.0995 | 0.3716 | 0.1926 | −0.0126 | 0.0048 |
| ABHD5 | 0.0844 | 0.0797 | 0.0436 | −0.0357 | −0.1067 | 0.4361 | 0.1074 | 0.3032 |
| OXTR | −0.0067 | 0.1542 | −0.0184 | 0.0557 | 0.5504 | 0.0000 | 0.7617 | 0.0000 |
| RPS12 | −0.3556 | 0.5379 | −0.2280 | 0.2149 | −0.1577 | −0.2829 | 0.3101 | 0.3117 |
| ANXA11 | −0.2290 | 0.1521 | −0.1075 | 0.1310 | 0.0353 | 0.1624 | 0.1786 | 0.1908 |
| SF3A1 | −0.0635 | 0.6275 | −0.0455 | 0.0745 | 0.1981 | 0.1926 | 0.2477 | 0.0356 |
| CCDC40 | −0.0663 | 0.7567 | −0.0633 | 0.0719 | 0.0827 | −0.1229 | 0.3419 | 0.0665 |
| SCO2 | −0.1063 | 0.7627 | −0.0633 | 0.1395 | 0.6676 | 0.0142 | 0.2453 | 0.2320 |
| SPRR2A | −0.1152 | −0.0412 | 0.0421 | −0.0180 | 0.1112 | −0.0159 | −0.0243 | 0.1876 |
| ACAT2 | 0.0946 | 0.0899 | 0.0173 | −0.0058 | 0.0002 | −0.0297 | 0.4778 | 0.2578 |
| P4HB | −0.2949 | −0.0219 | 0.0716 | 0.0973 | 0.0271 | 0.4695 | −0.0785 | 0.2816 |
| NFKBIA | 0.5919 | 0.6815 | −0.1598 | 0.2418 | 0.4237 | 0.6101 | 0.0563 | −0.2322 |
| C6orf132 | −0.2082 | −0.0122 | 0.1031 | −0.0632 | 0.1284 | 0.2382 | 0.0987 | 0.4475 |
| PSMD2 | 0.1867 | 0.2907 | 0.0679 | −0.0632 | 0.1823 | 0.3012 | 0.0011 | 0.3458 |
| CEBPD | 0.1257 | 0.3696 | −0.1658 | 0.0225 | 0.1969 | −0.0227 | 0.2809 | 0.4792 |
| RPL32 | −0.1435 | 0.2959 | −0.2163 | 0.1407 | −0.2101 | 0.3493 | 0.3720 | 0.2715 |
| ZFAND5 | 0.0037 | 0.5379 | 0.0358 | 0.0317 | −0.0626 | 0.0444 | 0.0760 | 0.3160 |
| CHD4 | −0.1044 | 0.5116 | 0.2462 | 0.4175 | 0.0469 | −0.0231 | −0.0197 | 0.1872 |
| POR | −0.1894 | 0.1130 | 0.0800 | 0.2579 | 0.0401 | 0.2003 | −0.0164 | 0.3949 |
| MT-ATP6 | −0.3318 | 0.1556 | 0.1954 | 0.9947 | 0.0795 | −0.0393 | 0.0576 | 0.3177 |
| EIFZAK2 | −0.0798 | 0.7020 | −0.0671 | 0.0844 | 0.1349 | −0.0365 | −0.0632 | 0.2630 |
| TUBB4B | −0.0971 | 1.3679 | 0.2701 | 0.1547 | 0.1694 | 0.4919 | 0.2160 | 0.4849 |
| AKR1C2 | −0.6241 | 0.7441 | −0.0506 | 0.0712 | 0.0574 | 0.3555 | 0.6171 | 0.1229 |
| RBP1 | 0.1865 | 0.0945 | −0.0092 | −0.0239 | 0.1103 | −0.0473 | 0.5486 | 0.0982 |
| SPRR2E | 0.0401 | −0.0005 | 0.0179 | 0.0000 | 0.0567 | 0.0000 | −0.0069 | −0.0141 |
| SPRR1B | −0.1170 | −0.0005 | 0.0260 | −0.0357 | −0.0094 | 0.1646 | −0.0377 | 0.0375 |
| PSMD11 | 0.1905 | 0.2120 | −0.0265 | 0.0230 | 0.0002 | 0.1550 | 0.1909 | 0.2088 |
| BAG1 | −0.0018 | −0.1192 | 0.0235 | 0.1198 | 0.0477 | 0.7105 | 0.1998 | 0.4442 |
| CYP1B1 | 0.0409 | −0.0142 | −0.0092 | −0.0060 | −0.0189 | −0.0150 | 0.2077 | 0.2701 |
| SAMD9 | −0.1296 | −0.0219 | 0.3203 | 0.0282 | 0.0749 | 0.3626 | −0.0218 | 0.0270 |
| ALDH1A3 | −0.2607 | 0.1820 | −0.0544 | 0.0347 | 0.0002 | 0.5700 | −0.0135 | 0.6404 |
| OGFR | −0.0771 | 0.7121 | −0.0092 | 0.0584 | 0.2296 | 0.2908 | 0.2836 | 0.2630 |
| NLRC5 | −0.0784 | 0.1340 | −0.0275 | −0.0532 | 0.0856 | 0.2401 | −0.0053 | 0.0186 |
| PFKP | 0.1851 | 0.4946 | −0.0275 | 0.0000 | −0.1428 | 0.0406 | 0.3808 | 0.5579 |
| PARP9 | 0.1848 | 0.4764 | −0.0265 | 0.0824 | 0.0002 | 0.5741 | 0.0519 | 0.1769 |
| CEACAM6 | −0.7234 | 0.1302 | 0.0671 | 0.0230 | 0.1577 | −0.0238 | −0.1164 | 0.2941 |
| MYO5B | −0.0769 | 0.2623 | −0.0864 | 0.1508 | 0.1871 | 0.0946 | 0.0949 | 0.2686 |
| STAT3 | −0.2612 | 0.4273 | 0.2144 | 0.0263 | 0.0757 | 0.2176 | 0.1514 | 0.0842 |
| SLC4A11 | −0.2254 | 0.3283 | 0.1210 | 0.0172 | −0.1428 | 0.3910 | 0.3498 | 0.3196 |
| KLF6 | 0.2487 | 0.6159 | −0.1285 | 0.3554 | 0.1785 | 0.3100 | −0.2535 | 0.8534 |
| CCDC57 | 0.0002 | 0.8295 | 0.1081 | −0.0340 | 0.0602 | 0.0115 | 0.0596 | 0.1019 |
| TRIM8 | 0.0291 | 0.5472 | 0.1210 | 0.0902 | 0.0479 | 0.2746 | 0.2417 | 0.1842 |
| MSMB | −0.1039 | 0.0637 | −0.3072 | 0.1389 | 0.2539 | 0.6868 | −0.1680 | −0.3908 |
| SPRR3 | −0.3851 | 0.0252 | −0.1269 | 0.0000 | 0.2101 | 0.9556 | 0.0500 | 0.2908 |
| TMPRSS4 | −0.2249 | 0.4390 | −0.0489 | −0.0175 | 0.1892 | 0.4118 | 0.0816 | 0.1218 |
| MVK | 0.1872 | 0.6078 | 0.1031 | −0.0357 | 0.0002 | 0.0544 | 0.1740 | −0.0721 |
| CTSH | −0.1509 | 0.7185 | 0.0506 | 0.1282 | 0.1421 | 0.0000 | 0.6886 | −0.0972 |
| KRT4 | 0.2957 | −0.0412 | 0.0748 | −0.0167 | 0.0508 | −0.0780 | −0.4625 | 0.5597 |
| RPL37 | −0.1920 | 0.3866 | −0.0137 | −0.0160 | 0.1289 | 0.0280 | 0.4006 | 0.3093 |
| ASRGL1 | −0.2185 | 0.3238 | 0.0086 | 0.0339 | −0.3811 | 0.5331 | 0.1933 | 0.4337 |
| CSNK1D | −0.2692 | 0.4376 | 0.0679 | 0.0614 | 0.3183 | 0.4735 | 0.3061 | −0.1449 |
| KRT7 | −0.2242 | −0.2235 | −0.1926 | −0.0100 | 0.1033 | 0.6119 | 0.0216 | 0.4396 |
| S100A14 | −0.3716 | 0.0195 | 0.0235 | 0.1141 | 0.1071 | 0.1085 | 0.2112 | 0.4107 |
| MACC1 | 0.1386 | 0.3138 | 0.1886 | −0.0265 | 0.0222 | 0.0336 | 0.0378 | 0.1065 |
| MT-CO2 | −0.3568 | 0.3983 | 0.1599 | 0.7929 | 0.0611 | −0.0108 | 0.4803 | 0.2335 |
| ARHGAP5 | 0.0626 | 0.4099 | −0.0238 | 0.0561 | −0.1180 | 0.3415 | 0.1775 | 0.5588 |
| TRIM29 | −0.2687 | 0.2274 | −0.0086 | −0.0284 | 0.2507 | 0.0786 | 0.2132 | 0.4842 |
| MUC1 | −0.5294 | 0.5577 | −0.3732 | 0.3792 | 0.4236 | 0.7388 | 0.2071 | 0.4049 |
| SLC25A3 | −0.1028 | 0.3567 | −0.0077 | 0.2630 | 0.3271 | 0.1979 | 0.2112 | 0.4638 |
| PYGL | 0.1595 | 0.2665 | 0.0794 | 0.0804 | 0.1412 | 0.1186 | 0.2246 | 0.3312 |
| MUC20-OT1 | −0.1000 | 0.9435 | 0.1210 | 0.0712 | 0.0981 | 0.0336 | 0.2336 | −0.0436 |
| ACTN4 | 0.0232 | 0.5775 | 0.0634 | 0.0157 | −0.2412 | 0.2335 | 0.1465 | 0.3695 |
| S100A10 | −0.0898 | 1.1439 | −0.0550 | 0.0458 | 0.2676 | 0.0255 | 0.4584 | 0.2332 |
| KRT24 | −0.3786 | −0.0005 | 0.1031 | 0.0269 | 0.0567 | 0.7776 | −0.0539 | −0.1914 |
| GAN | −0.1607 | 0.2714 | 0.0260 | 0.0230 | −0.0946 | 0.2401 | −0.0130 | 0.4429 |
| GLUL | −0.2134 | 0.6235 | 0.0220 | 0.0697 | −0.0491 | 0.0704 | 0.0101 | 0.3409 |
| IFI44L | −0.0041 | 0.9156 | −0.0275 | 0.0844 | 0.5851 | 0.1587 | 0.2892 | 0.2077 |
| RDH10 | −0.4027 | 0.4133 | −0.0653 | 0.1812 | 0.1783 | 0.3074 | −0.0654 | 0.3339 |
| KRT19 | −0.4907 | 0.4057 | −0.0630 | 0.3816 | −0.1558 | 0.3115 | −0.0040 | 0.4215 |
| EZR | −0.1786 | 1.0040 | 0.0809 | 0.2525 | 0.0593 | 0.3413 | 0.1299 | 0.2198 |
| IFITM1 | −0.2943 | 0.2768 | −0.0177 | 0.0117 | 0.2717 | 0.1605 | 0.7030 | 0.6526 |
| FLNB | −0.0636 | 0.3393 | 0.1031 | 0.0745 | 0.0723 | −0.2130 | 0.2782 | 0.2796 |
| S100P | −0.5590 | 0.6666 | −0.0247 | 0.1206 | 0.4393 | −0.1728 | 0.0302 | −0.7573 |
| MT-ND5 | 0.2931 | 0.3066 | 0.1674 | 0.7394 | −0.2728 | 0.2290 | 0.4204 | 0.7959 |
| AHNAK2 | 0.0068 | 0.7271 | −0.0365 | −0.0112 | −0.0867 | −0.0740 | 0.7479 | 0.0925 |
| TNFAIP3 | 0.1755 | 0.3997 | −0.2609 | 0.4044 | 0.9857 | −0.0875 | 0.2776 | 0.0255 |
| TBC1D8 | 0.2123 | 0.9606 | 0.0885 | −0.0228 | 0.3096 | 0.0134 | 0.4109 | 0.0385 |
| S100A9 | −0.7110 | 0.0945 | 0.3577 | 0.5794 | 0.0594 | 1.1365 | 0.0967 | −0.1185 |
| OS9 | −0.1803 | −0.0196 | −0.0780 | 0.0000 | 0.2860 | 0.2734 | −0.1642 | 0.6389 |
| S100A6 | −0.1294 | 0.9265 | −0.3455 | 0.4842 | 0.6425 | 0.2787 | 0.6162 | 0.7700 |
| CEACAM5 | −0.3491 | 0.0463 | 0.0421 | 0.0687 | −0.0091 | 0.5963 | 0.0162 | 0.0603 |
| SPINT2 | −0.0430 | −0.0971 | 0.0740 | 0.1038 | 0.0460 | 0.4210 | 0.2513 | 0.5649 |
| ACTG1 | 0.0777 | 0.5939 | 0.0584 | 0.0920 | 0.0942 | 0.6051 | 0.2691 | 0.6787 |
| MT-CYB | −0.0975 | 0.0889 | 0.2135 | 1.1338 | 0.2461 | 0.0586 | 0.1177 | 0.5854 |
| MT-ND1 | −0.0529 | 0.4429 | −0.0039 | 1.2174 | 0.9417 | −0.2427 | 0.2240 | 0.6913 |
| MUC21 | −0.2076 | −0.0142 | 0.0544 | −0.0060 | 0.0002 | −0.1155 | −0.0209 | 0.1984 |
| MT-CO3 | −0.2190 | 0.3234 | 0.2570 | 0.4338 | 0.7821 | 0.1623 | 0.4989 | 0.0465 |
| LRRFIP1 | −0.0836 | 0.6429 | 0.2020 | 0.0824 | −0.0073 | 0.3015 | 0.2416 | 0.2514 |
| PPL | 0.1027 | 0.4499 | 0.0083 | −0.0507 | 0.1958 | 0.6728 | −0.1055 | 0.4292 |
| ELF3 | −0.2093 | 0.7759 | 0.0317 | 0.5805 | 0.3521 | 0.7637 | 0.2565 | 0.6241 |
| MX1 | −0.1104 | 0.9056 | 0.0879 | 0.4015 | 0.3026 | 0.0599 | 0.1261 | 0.6397 |
| MTRNR2L1 | −0.2457 | 0.7486 | 0.0194 | 0.7470 | 0.3989 | 0.8319 | 0.4662 | −0.0122 |
| F3 | 0.1518 | 0.1873 | 0.1125 | 0.1451 | −0.1773 | −0.1220 | −0.2703 | 0.7578 |
| MTRNR2L12 | 0.2930 | 0.5502 | −0.2569 | 1.7414 | 0.7236 | −0.2296 | 0.4073 | 0.0178 |
| WFDC2 | −0.4447 | 0.3381 | 0.2753 | 0.2673 | −0.0502 | 0.4337 | 0.0632 | 0.6150 |
| MT-ND6 | 0.3273 | 0.5029 | 0.2812 | −0.1016 | 0.0866 | 0.1022 | 0.3444 | 0.1097 |
| PER1 | −0.0183 | 0.3249 | 0.0089 | 0.3821 | 0.6183 | 0.4695 | 0.3556 | 0.0191 |
| PLEC | −0.1322 | 0.3547 | −0.0339 | 0.0054 | 0.4068 | 0.1203 | 0.1588 | 0.4664 |
| TACSTD2 | −0.1726 | 0.9665 | 0.0551 | 0.1649 | −0.1833 | 0.2075 | 0.2265 | 0.5361 |
| LMO7 | −0.2075 | 0.4274 | 0.0286 | 0.1076 | −0.0175 | 0.3191 | 0.2816 | 0.0411 |
| AHNAK | −0.0094 | 0.5564 | 0.2901 | 0.2944 | −0.1284 | 0.4949 | 0.0295 | 0.1232 |
| IFI27 | −0.1209 | 0.7761 | 0.0310 | 0.7600 | 0.1883 | 0.4861 | 0.3087 | 0.5085 |
| IFITM3 | −0.3798 | 0.5314 | 0.0634 | 0.1643 | 0.4427 | 0.0893 | 0.7705 | 0.4493 |
| LGALS3 | −0.2468 | 0.9224 | 0.0000 | −0.0042 | 0.0222 | 0.8931 | 0.3679 | 0.2933 |
| PSCA | −0.4441 | 0.4891 | 0.1359 | 0.5285 | 0.3286 | 1.0605 | 0.0689 | 0.6666 |
| IF16 | −0.2621 | 0.6892 | 0.1220 | 0.2725 | 0.4469 | 1.2572 | 0.6535 | 0.5369 |
| MUC5AC | −0.4601 | 0.1692 | 0.0406 | 0.0768 | −0.0175 | 1.8219 | −0.0136 | 0.0860 |

| | | | | Early Response | | | | SPRR2D high |
|---|---|---|---|---|---|---|---|---|
| | Squamous | Cilia high | Deuterosomal | FOXJ1 high | Early Response | SCGB1A1 high | Secretory | Squamous |
| Gene | Cells | Ciliated Cells | Cells | Ciliated Cells | Secretory Cells | Goblet Cells | Cells | Cells |
| SAA1 | 0.0956 | −0.1339 | −0.4150 | −1.0544 | 0.2370 | −0.0991 | −0.6724 | −0.2996 |
| CD74 | 0.0052 | −0.0834 | 0.4150 | −2.8755 | −0.2185 | −0.0895 | 0.0914 | 0.1476 |
| HLA-DRB1 | 0.1612 | 0.0375 | −0.1155 | −1.7021 | −0.8021 | −0.0219 | −0.2630 | 0.3312 |
| HSPA1A | −0.1876 | −0.3145 | −1.1775 | −0.4468 | 0.2716 | 0.8339 | −0.9363 | 0.0489 |
| HLA-DRB5 | −0.0418 | −0.0199 | −0.0780 | −0.4695 | −0.4800 | −0.4582 | 0.0671 | 0.0000 |
| HLA-DRA | −0.0339 | −0.0780 | −0.2224 | −1.9434 | 0.0280 | −0.3325 | 0.3370 | 0.0902 |
| APRT | −0.0289 | 0.0000 | −0.2895 | −0.5399 | −0.7997 | −0.5480 | −0.3650 | −0.2035 |
| MMP10 | 0.0825 | 0.0000 | −1.1339 | 0.0000 | −1.5096 | 0.1144 | −0.1903 | 0.1375 |
| CCDC171 | 0.0577 | −0.2352 | −0.9115 | −0.3955 | −0.3081 | −0.0251 | −0.0931 | −0.0238 |
| CDKN1A | −0.0365 | −0.0395 | −0.3081 | −0.3236 | 0.7341 | −0.3686 | −0.6881 | −1.8580 |
| RARRES1 | 0.3301 | −0.0199 | 0.1756 | −0.1476 | −0.6462 | −0.7959 | −0.6705 | 0.1375 |
| LDLRAD1 | −0.1339 | −0.3145 | 0.0348 | −1.9889 | 0.0931 | −0.0181 | 0.0000 | 0.6724 |
| HTATSF1 | −0.0623 | −0.1876 | 0.1069 | −0.6621 | −1.0171 | −0.2790 | −0.3819 | −0.2224 |
| FMO3 | −0.2451 | −0.3728 | −0.4448 | −2.0302 | −0.4104 | 0.0622 | 0.7687 | 2.0544 |
| EIF4G2 | −0.0949 | −0.2563 | −0.6818 | −0.5871 | −0.7863 | 0.5516 | −0.3334 | 0.0733 |
| HSPA1B | −0.3978 | 0.0000 | −0.6818 | −0.9137 | 0.4023 | 0.7049 | −0.3604 | −0.5580 |
| TP53BP1 | −0.0690 | −0.4263 | −0.0671 | −0.3251 | −0.3219 | −0.2849 | 0.1476 | 0.2410 |
| PLAT | −0.1089 | −0.0199 | 0.0000 | −0.1069 | 0.0000 | 0.0000 | −0.3026 | −1.8628 |
| FCGBP | 0.0504 | −0.0589 | −0.1155 | −0.1069 | −0.2801 | −0.1720 | −0.0431 | −0.9758 |
| CCDC113 | 0.2566 | −0.4425 | −0.3536 | −1.5019 | 0.1587 | −0.0301 | −0.0931 | 0.1137 |
| UGDH | −0.1454 | −0.0199 | −0.2224 | 0.0869 | −0.7225 | −0.0036 | 0.1699 | −0.1375 |
| NWD1 | 0.1660 | −0.1783 | −0.1876 | −0.8509 | −0.2224 | −0.1557 | −0.1375 | 0.2392 |
| RPS6 | −0.3536 | −0.3440 | −0.4854 | −1.4444 | −0.0589 | 0.5104 | 0.3575 | −0.3565 |
| FAM216B | −0.0536 | −0.1069 | −0.1155 | −1.8783 | −0.0339 | −0.0361 | 0.0000 | 0.3547 |
| RPS19 | −0.0637 | −0.2395 | −0.8413 | −1.4310 | −0.3034 | 0.5541 | 0.6314 | −0.6285 |
| SEC14L3 | −0.0780 | 0.1420 | −0.0395 | −1.6759 | −0.1312 | −0.1557 | 0.0000 | 0.2392 |
| LGALS8 | −0.0605 | −0.0395 | −0.1312 | −1.1069 | −0.3099 | 0.7224 | −0.0544 | −0.3728 |
| TCP1 | −0.1031 | −0.0896 | 0.1402 | −1.0302 | −0.3479 | −0.2790 | −0.3058 | −0.2370 |
| PGM2L1 | 0.0713 | −0.1699 | −0.5025 | −0.0544 | −0.8931 | 0.0370 | 0.1476 | −0.0704 |
| NFIA | −0.1155 | 0.0173 | −0.4739 | −0.7425 | −0.2895 | 0.3353 | 0.0186 | −0.2016 |
| MTURN | −0.0758 | −0.2395 | −0.1155 | −1.3440 | 0.0740 | −0.3827 | −0.0931 | −0.3479 |
| TXNL4A | −0.0487 | 0.0759 | −0.2224 | −0.5519 | −0.3219 | −0.0340 | 0.0173 | −0.3819 |
| CCNO | −0.0371 | −0.0184 | −0.7638 | 0.0451 | −0.8580 | 0.0654 | 0.0000 | 0.0902 |
| ATF3 | −0.3823 | −0.0780 | 0.0000 | 1.4481 | −2.0780 | 0.2039 | −0.4150 | −0.4634 |
| HLA-DPA1 | −0.1898 | 0.0000 | −0.0780 | −0.6070 | −0.1964 | 0.2277 | −0.0931 | 0.0000 |
| TSGA10 | −0.0623 | −0.1783 | −0.2801 | −0.9201 | −0.2515 | −0.0785 | −0.0704 | 0.1137 |
| ERLEC1 | 0.2352 | −0.2395 | −0.0297 | −0.9475 | −0.0506 | 0.8828 | −0.3536 | −0.5025 |
| SCGB1A1 | −0.1832 | 0.0000 | 0.0704 | −0.1069 | 0.3547 | −1.1054 | −0.1699 | 0.0000 |
| ALOX15 | 0.1282 | −0.0671 | −0.3458 | −1.0045 | 0.0766 | −0.7149 | −0.6114 | 0.6542 |
| BRD7 | −0.0571 | −0.1520 | −0.1876 | −0.9169 | −0.3692 | 0.2864 | −0.0589 | −0.5194 |
| XBP1 | 0.2693 | −0.0184 | −0.0641 | −0.5399 | −0.4481 | 0.4016 | −0.8037 | −0.1468 |
| PLCB4 | −0.0395 | −0.4623 | −0.0339 | −1.0544 | 0.2157 | −0.3443 | 0.2251 | −0.0238 |
| BRD8 | 0.1434 | −0.2382 | −0.3847 | −0.2768 | −0.7574 | −0.1831 | −0.2630 | −0.0506 |
| BAZ1B | −0.0958 | 0.2313 | −0.1876 | −0.5168 | −0.0280 | −0.2281 | 0.0885 | −0.4903 |
| HSPH1 | 0.1903 | −0.3370 | 0.6007 | −0.5755 | −1.2876 | 0.3675 | −0.0104 | 0.0825 |
| CEP83 | 0.0142 | 0.0097 | −0.3356 | −0.2630 | −0.1926 | −0.2040 | 0.0220 | −0.0473 |
| RSRP1 | 0.1470 | −0.4695 | 0.1587 | −0.3492 | −0.1203 | −0.5371 | −0.2538 | −0.3692 |
| ADH1C | 0.0059 | −0.1339 | 0.1618 | −1.6998 | −0.5976 | 0.2030 | −0.2322 | 0.1699 |
| PPP1R7 | −0.1054 | −0.1876 | 0.0704 | −0.6919 | −0.4349 | 0.2343 | −0.1069 | −0.3985 |
| SMC4 | 0.0134 | −0.0589 | −0.2630 | −0.0101 | −0.3969 | 0.9094 | 0.2251 | −1.3565 |
| DOCK1 | −0.0589 | −0.1409 | −0.2515 | −0.7105 | 0.1375 | 0.0518 | −0.1592 | −0.3026 |
| DZIP3 | 0.0450 | −0.1484 | −0.0395 | −1.2866 | −0.1926 | 0.1599 | −0.0473 | 0.1476 |
| ECI1 | 0.0489 | −0.0395 | −0.1876 | −1.0138 | −0.1575 | −0.0455 | −0.0199 | −0.5194 |
| NDFIP1 | 0.1190 | 0.0375 | −0.1155 | −0.9642 | −0.2224 | −0.3443 | −0.1876 | −0.1417 |
| ACAA1 | −0.1085 | −0.0589 | −0.1876 | −0.3175 | −0.1240 | 0.4423 | −0.0217 | −0.2224 |
| TOR1AIP2 | −0.1361 | −0.0589 | −0.1155 | −0.2926 | −0.1651 | 0.3906 | 0.0772 | −0.3155 |
| RPS27 | −0.1908 | 0.0956 | −0.1699 | −0.5399 | −0.4150 | 0.5813 | 0.1099 | −0.3650 |
| ACTR3 | −0.0395 | −0.0589 | −0.0780 | −0.7258 | 0.0525 | 0.0011 | −0.1375 | −0.6222 |
| ZFC3H1 | 0.0475 | −0.4279 | −0.1876 | −0.5399 | −0.4657 | −0.0817 | 0.1635 | −0.3870 |
| PNRC1 | −0.1054 | 0.1444 | −0.2224 | 0.2086 | 0.0000 | −0.2986 | −0.1844 | −0.7796 |
| OSBPL6 | −0.0071 | 0.2295 | 0.0000 | −1.2195 | 0.0000 | −0.0361 | 0.0000 | 0.0000 |
| KIAA0100 | −0.0431 | −0.2224 | −0.1876 | −0.1575 | −0.1454 | 0.5087 | 0.2426 | −0.4507 |
| IGFBP5 | 0.3076 | −0.2224 | 0.0000 | −1.2768 | −0.2224 | 0.3418 | 0.1375 | −0.0238 |
| UPF3B | −0.0501 | −0.2934 | −0.1312 | −0.4024 | −0.4150 | 0.2121 | −0.0849 | 0.0000 |
| HLA-DQB1 | −0.0289 | 0.0000 | −0.0395 | −0.2996 | −0.1375 | −0.0625 | 0.0000 | 0.0000 |
| CARS2 | −0.0217 | −0.1699 | 0.2224 | −0.5399 | −0.5384 | −0.0699 | −0.2016 | −0.0473 |
| PLEKHA5 | 0.2696 | −0.5397 | −0.1430 | −0.5184 | −1.0732 | 0.5601 | −0.2035 | −0.0589 |
| LRRC46 | −0.0780 | −0.1664 | 0.0704 | −1.3440 | 0.0000 | 0.0000 | −0.0238 | −0.0238 |
| GCHFR | −0.0069 | −0.0199 | −0.1876 | −0.3320 | −0.6439 | 0.0242 | −0.0217 | −0.3410 |
| NISCH | 0.0138 | −0.1876 | −0.3219 | −0.0544 | −0.2451 | 0.1164 | −0.0641 | −0.1699 |
| HES6 | −0.0371 | 0.0000 | −0.6930 | −0.9024 | −0.2224 | −0.0464 | 0.0000 | −0.0704 |
| TSTA3 | 0.1038 | −0.0395 | −0.0395 | −0.7105 | −0.3326 | −0.6041 | 0.0324 | −0.5025 |
| BTN3A2 | −0.1054 | 0.0000 | 0.0000 | −1.1476 | 0.1038 | 0.0647 | −0.0217 | −0.0641 |
| AK2 | −0.0571 | −0.0199 | −0.1876 | −1.0544 | −0.4854 | 0.1854 | −0.2775 | −0.4634 |
| ZNF292 | −0.1186 | −0.1575 | −0.1520 | 0.4664 | 0.0161 | 0.2005 | 0.0375 | −1.1015 |
| PTPN3 | 0.0780 | −0.2395 | 0.1069 | −0.3540 | −0.2563 | −0.1047 | −0.0849 | −0.3458 |
| MSH3 | 0.0000 | −0.3692 | −0.4150 | −0.4695 | −0.3626 | 0.3927 | 0.1038 | 0.0000 |
| EFCAB10 | −0.0339 | −0.2395 | −0.2563 | −1.6070 | 0.0000 | 0.0000 | 0.0000 | −0.0238 |
| PSMD1 | −0.0969 | 0.0704 | 0.1587 | −0.1800 | −0.3293 | 0.1002 | 0.2182 | −0.6421 |
| GPR162 | 0.0504 | −0.1409 | −0.0780 | −0.5201 | −0.0339 | −0.0181 | 0.1375 | −0.0238 |
| ANAPC5 | 0.0438 | −0.2515 | 0.0931 | −0.4330 | 0.1881 | 0.0825 | 0.1881 | −0.0589 |
| PIP | 0.1402 | 0.0000 | 0.0000 | −0.5475 | −0.0339 | 0.0000 | −0.0238 | −1.9241 |
| DENND2C | 0.0208 | −0.2895 | −0.1520 | 0.4431 | −0.3923 | 0.2150 | 0.1476 | −0.5443 |
| INTS3 | −0.0349 | −0.0995 | −0.1520 | −0.2768 | −0.0589 | −0.1557 | −0.0931 | −0.0704 |
| NUP50 | 0.0475 | 0.0000 | −0.3847 | −0.4330 | −0.3293 | −0.0689 | 0.1168 | 0.0544 |
| PPIL6 | −0.1285 | −0.0671 | −0.1520 | −0.7914 | −0.0339 | −0.0361 | −0.0473 | 0.0000 |
| TNFSF10 | −0.0821 | −0.1520 | −0.3949 | −1.2580 | −0.4482 | 0.4097 | −0.0732 | −0.8497 |
| RPS4X | −0.3668 | −0.0184 | −0.1993 | −1.0324 | −0.1907 | 0.3812 | 0.3769 | −0.3293 |
| SUN1 | 0.1069 | −0.4657 | −0.3536 | −0.8716 | 0.0489 | 0.2355 | 0.2792 | −0.6062 |
| PSMB6 | 0.0338 | 0.1829 | −0.3847 | −1.0544 | −0.1349 | −0.0799 | −0.0150 | 0.0995 |
| TRIM24 | −0.0875 | −0.1155 | −0.4739 | 0.6236 | −0.8413 | 0.0923 | 0.1255 | −0.0780 |
| CAB39 | 0.0529 | −0.1339 | −0.3219 | −0.7325 | −0.5619 | −0.0487 | −0.2730 | −0.2102 |
| FAM111A | 0.0000 | −0.3219 | −0.1520 | −0.6711 | −0.1592 | −0.4105 | −0.1806 | 0.3262 |
| RHOV | 0.1183 | 0.0000 | 0.1829 | −0.6070 | −0.1375 | −0.2881 | 0.2439 | −0.5949 |
| SSR3 | 0.0214 | −0.0199 | −0.1155 | −0.6394 | −0.6301 | 0.4165 | −0.1699 | −0.5025 |
| HSD17B13 | 0.0202 | 0.1155 | 0.0000 | −0.7083 | −0.0297 | −0.1405 | −0.0704 | 0.0000 |
| HNRNPH3 | 0.0475 | 0.0000 | −0.1312 | −0.0544 | −0.0849 | −0.3686 | −0.1339 | 0.1255 |
| WDR78 | 0.0427 | −0.1375 | 0.0000 | −0.8845 | 0.1587 | −0.0361 | −0.0473 | 0.2157 |
| CDK2AP2 | −0.1015 | −0.0395 | −0.4739 | −0.5399 | 0.2854 | −0.1812 | 0.4695 | −0.2410 |
| CFB | −0.0501 | −0.0589 | −0.2224 | −0.5399 | 0.2251 | −0.0817 | 0.0324 | −0.7370 |
| MLEC | −0.0141 | −0.1339 | 0.1255 | −1.7325 | −0.1699 | 0.3418 | 0.1850 | −0.0217 |
| ATP5F1D | −0.0444 | −0.0969 | 0.0000 | −0.6919 | −0.2479 | −0.1443 | 0.4436 | −0.0780 |
| NARS | 0.0630 | −0.2845 | −0.6374 | −0.9024 | −0.0544 | 0.4895 | −0.1623 | −0.4150 |
| CCDC74A | −0.1054 | −0.0544 | −0.4406 | −1.0220 | 0.0000 | −0.0361 | 0.1375 | −0.0238 |
| KRI1 | −0.0517 | 0.0186 | −0.1876 | −0.3955 | −0.4150 | −0.0943 | −0.1155 | −0.2429 |
| OMG | −0.2051 | −0.1155 | 0.2224 | −2.1277 | −0.0339 | 0.0895 | −0.0238 | 0.0902 |
| GRAMD2B | −0.0444 | −0.0395 | −0.2563 | −0.0910 | −0.0297 | 0.0212 | −0.0589 | −0.3985 |
| MPV17L | 0.0825 | −0.0780 | −0.1155 | −0.8845 | 0.0946 | −0.0136 | −0.4150 | −0.1155 |
| CMPK1 | −0.0623 | 0.0759 | −0.3081 | −1.5690 | −0.4507 | 0.3740 | 0.0000 | −0.0627 |
| C1orf43 | −0.0339 | −0.0395 | −0.1876 | −0.9613 | −0.7370 | 0.4836 | −0.2395 | −0.3440 |
| RPS27L | −0.1024 | 0.0000 | −0.0671 | −0.7590 | 0.3785 | −0.2600 | −0.5995 | −0.8974 |
| SLC28A3 | −0.0360 | −0.0184 | −0.2224 | −0.2580 | −1.1155 | 0.1219 | −0.0704 | −0.0473 |
| NACA | −0.2056 | −0.1520 | −0.1520 | −0.9720 | −0.4914 | 1.1034 | −0.3004 | −0.3536 |
| LY6D | −0.2542 | 0.0000 | 0.0000 | −0.1069 | −0.7033 | 0.5623 | −0.4634 | −0.1454 |
| MRFAP1L1 | −0.0349 | −0.0780 | −0.1520 | −0.0544 | −0.4546 | 0.4384 | −0.0641 | −0.3985 |
| LCORL | −0.0297 | −0.0199 | −0.1520 | −0.3870 | −0.2801 | −0.2040 | −0.0473 | −0.0431 |
| NBEAL1 | 0.0802 | −0.1240 | 0.0614 | −0.4695 | 0.0566 | 0.0048 | −0.4150 | −0.0995 |
| GBP3 | −0.1585 | −0.0671 | −0.1520 | −0.7914 | 0.1476 | 0.1814 | 0.1635 | −0.3306 |
| AK9 | 0.1022 | −0.8745 | −0.1430 | −0.0736 | 0.4898 | −0.1720 | −0.0704 | 0.0000 |
| DPM2 | −0.0517 | −0.0395 | 0.0000 | −0.4695 | −0.3890 | −0.0402 | −0.0473 | 0.0759 |
| TMX3 | −0.0289 | −0.1155 | −0.0780 | −0.8231 | −0.5619 | −0.0625 | 0.1357 | 0.0000 |
| DNAI1 | 0.0270 | −0.3099 | −0.1520 | −0.8940 | 0.0000 | 0.1255 | 0.0000 | −0.0704 |
| LCOR | −0.0460 | −0.1339 | −0.3536 | −0.5070 | −0.6804 | 0.2984 | 0.7045 | 0.1850 |
| USP46 | 0.0849 | −0.0589 | −0.1155 | −0.4024 | −0.5670 | −0.0207 | 0.0825 | −0.0217 |
| PHIP | 0.2820 | −0.3672 | −0.7843 | −0.0220 | 0.0286 | 0.0267 | 0.5070 | −0.2109 |
| ANKS1A | 0.0138 | −0.3379 | −0.2563 | −0.0220 | −0.3219 | −0.1253 | 0.2157 | −0.1454 |
| SLC25A24 | 0.0073 | −0.0969 | −0.1876 | −0.1800 | −0.5384 | 0.2453 | −0.1844 | 0.0000 |
| NUDT3 | −0.0641 | −0.0184 | −0.2224 | 0.2504 | −0.4150 | 0.0630 | −0.1454 | −0.0473 |
| ST6GALNAC1 | 0.0262 | −0.3145 | 0.0614 | −0.7258 | −0.3536 | 0.4334 | −0.1926 | −0.4507 |
| BUB3 | −0.0349 | −0.1339 | −0.1520 | −0.2065 | −0.3458 | 0.1074 | −0.1651 | −0.2035 |
| SLC35A2 | −0.0661 | 0.0956 | 0.0000 | −0.7649 | −0.4903 | −0.1881 | 0.0902 | −0.2630 |
| DDX17 | 0.1821 | 0.0208 | −0.3112 | −0.7992 | −0.0280 | −0.0337 | −0.2763 | 0.2713 |
| WFDC6 | −0.0145 | −0.0199 | −0.1520 | −0.9434 | 0.0000 | 0.1255 | 0.1375 | 0.0000 |
| SSBP4 | 0.1038 | −0.1926 | −0.2563 | −0.5850 | −0.2801 | −0.1557 | 0.0000 | 0.1926 |
| TP53I11 | 0.0146 | 0.0000 | 0.0000 | −0.9434 | −0.1806 | 0.2277 | −0.1844 | 0.1137 |
| H3F3B | −0.3863 | 0.1168 | −0.3356 | 0.2025 | 0.1435 | −0.0703 | −0.5119 | 0.0343 |
| HINT1 | 0.0348 | −0.1339 | −0.1926 | −0.7761 | −0.0297 | 0.4267 | 0.0385 | −0.5305 |
| BTAF1 | −0.0067 | −0.3585 | −0.2895 | −0.7914 | −0.5850 | 0.4058 | 0.1926 | −0.3306 |
| TMEM123 | −0.2168 | 0.0525 | −0.2801 | −1.0709 | −0.8745 | 0.7162 | −0.0555 | −0.4681 |
| IFNAR1 | −0.0349 | −0.0589 | −0.0995 | −0.9024 | −0.2224 | 0.5548 | 0.0000 | −0.1520 |
| MED13L | −0.0849 | −0.1876 | −0.2895 | 0.3379 | 0.0000 | 0.0223 | −0.1155 | −0.3293 |
| CA12 | 0.0294 | −0.0395 | 0.0000 | −0.1069 | −0.3661 | −0.0407 | −0.2382 | −0.1375 |
| ZFYVE16 | −0.0141 | −0.0171 | −0.1876 | −0.3175 | 0.2370 | −0.0991 | −0.2630 | −0.0199 |
| ABO | −0.0780 | −0.0780 | −0.0780 | −0.2065 | −0.4150 | 0.1281 | −0.1375 | −0.0473 |
| CCT7 | −0.0501 | −0.0395 | −0.3219 | −0.3764 | −0.3692 | −0.0817 | 0.2996 | −0.0395 |
| ZSCAN31 | −0.0875 | −0.0199 | −0.1155 | −0.3955 | 0.0000 | 0.2423 | −0.0473 | −0.0217 |
| CACFD1 | −0.0073 | −0.0199 | −0.1520 | −0.6919 | −0.3219 | 0.4751 | −0.0431 | −0.5361 |
| NQO1 | −0.0931 | 0.0000 | −0.5619 | −0.6571 | 0.3561 | 0.3273 | −0.5850 | −0.0969 |
| FABP6 | −0.0211 | −0.3585 | −0.0780 | −0.2065 | 0.0000 | −0.0361 | −0.0238 | 0.1375 |
| ATF6 | −0.0487 | −0.0544 | −0.1876 | −0.5850 | −0.6027 | 0.1036 | 0.0885 | −0.2429 |
| GSTA2 | −0.2655 | 0.0186 | −0.0780 | −0.6919 | 0.0000 | 0.0000 | −0.0238 | 0.0000 |
| SUGT1 | −0.0711 | 0.2099 | −0.4448 | −0.5336 | 0.0444 | −0.1706 | −0.0395 | 0.0671 |
| RALBP1 | 0.0196 | −0.1876 | −0.3219 | −1.0862 | −0.4594 | 0.8541 | 0.4312 | −0.3728 |
| SYS1 | −0.0641 | −0.1339 | −0.2224 | −0.5284 | −0.2977 | 0.0691 | 0.2854 | −0.0431 |
| COMMD2 | −0.0217 | −0.0589 | −0.0780 | −0.3955 | −0.4657 | 0.3208 | −0.0931 | −0.1806 |
| SAA2 | 0.3247 | −0.0589 | −0.1155 | 0.0610 | −0.0297 | −0.1225 | 0.0704 | 0.1561 |
| MRPL49 | −0.0780 | 0.0000 | 0.0000 | −0.8231 | −0.3890 | −0.0251 | −0.1375 | −0.1592 |
| TEKT1 | −0.1339 | −0.2538 | −0.3847 | −0.7370 | 0.0000 | −0.1056 | −0.0238 | 0.0000 |
| PTPN13 | 0.0713 | 0.0000 | −0.3081 | 0.4374 | −0.2332 | 0.3208 | 0.4606 | −0.9780 |
| MRPL41 | −0.0671 | 0.0704 | −0.2895 | −1.0170 | −0.6202 | 0.4853 | 0.2224 | −0.3219 |
| CDC16 | −0.0917 | −0.1740 | 0.0704 | −0.9289 | 0.2370 | 0.1903 | −0.1651 | −0.0217 |
| LRRC43 | −0.0145 | −0.2895 | −0.0395 | −0.7105 | −0.0339 | 0.0000 | 0.1375 | 0.0000 |
| LUC7L3 | 0.0677 | −0.4561 | 0.2003 | −0.1369 | 0.0304 | 0.0281 | 0.2025 | −0.0375 |
| CTCF | −0.2029 | 0.0489 | −0.3536 | −0.1325 | −0.4150 | 0.0863 | 0.4557 | −0.0995 |
| CIRBP | −0.0531 | −0.1565 | 0.3833 | −0.5690 | −0.6545 | 0.8377 | 0.1110 | 0.1050 |
| EFCAB12 | 0.0286 | −0.3356 | −0.1876 | −0.9642 | 0.0000 | 0.0000 | 0.0000 | 0.1375 |
| SPR | −0.0517 | 0.0000 | −0.0395 | −0.6711 | 0.0000 | −0.1392 | 0.0000 | −0.0217 |
| ZNF664 | −0.3190 | 0.0885 | 0.1069 | −0.6759 | −0.3410 | 0.7200 | −0.0395 | −0.0217 |
| PHYKPL | −0.0289 | −0.0184 | 0.0000 | −0.5475 | −0.2801 | −0.1276 | −0.1155 | 0.0825 |
| HSPA9 | −0.0498 | −0.0721 | −0.6077 | −0.1325 | −0.3356 | 0.4038 | 0.4938 | −0.0896 |
| ANAPC13 | −0.0071 | −0.0395 | −0.0780 | −0.0544 | −0.3356 | −0.0846 | 0.1255 | −0.2829 |
| FYB2 | 0.0073 | −0.2361 | −0.0780 | −0.3955 | 0.4898 | −0.2661 | −0.0473 | 0.1137 |
| INSR | −0.0211 | 0.0186 | −0.0339 | −0.5764 | 0.2392 | 0.6173 | −0.1592 | −0.3379 |
| MFN2 | 0.0071 | −0.0969 | −0.1520 | −0.6394 | −0.6323 | 0.4836 | −0.1069 | −0.1844 |
| SRSF7 | 0.0908 | −0.1926 | −0.0589 | −0.1476 | −0.3306 | 0.0129 | −0.2895 | −0.3410 |
| DZIP1 | −0.0732 | −0.3112 | −0.2563 | −1.2580 | 0.1255 | −0.0538 | 0.0000 | −0.0238 |
| H2AFZ | −0.1220 | −0.0199 | −0.3081 | −0.6270 | −0.3058 | −0.3817 | −0.1155 | 0.5700 |
| PPA1 | 0.0142 | −0.0395 | 0.0000 | −0.6711 | −0.4150 | 0.3083 | −0.5749 | −0.1240 |
| EVI5 | −0.0280 | 0.0725 | −0.4739 | −0.5626 | −0.4406 | 0.3319 | −0.2224 | −0.3026 |
| RPL6 | −0.0360 | 0.0375 | −0.6881 | 0.0899 | −0.5119 | 0.5224 | −0.1478 | −0.2594 |
| PPP1CB | −0.1753 | 0.0186 | 0.0270 | −0.4919 | 0.2204 | 0.0536 | −0.3610 | −0.2869 |
| TSR3 | −0.0289 | 0.0956 | −0.1520 | −1.0544 | −0.1758 | 0.0499 | −0.1592 | −0.2630 |
| SPG21 | 0.0754 | −0.0969 | 0.0348 | −0.5764 | 0.3923 | 0.3849 | −0.2016 | −0.3306 |
| HMGB1 | −0.0071 | −0.1699 | −0.6114 | −0.1857 | 0.1476 | 0.3679 | 0.3626 | −0.3985 |
| PTPRT | 0.0294 | −0.0339 | −0.0395 | −0.6919 | −0.0339 | 0.0543 | −0.0931 | 0.0000 |
| ANKRD36 | −0.0280 | −0.5025 | −0.5236 | 0.3241 | 0.1255 | 0.1525 | −0.0641 | −0.3847 |
| THOC2 | −0.0891 | 0.3049 | −0.2730 | 0.2549 | −0.2370 | 0.6023 | 0.0772 | −0.2996 |
| GLYR1 | 0.0069 | −0.1155 | −0.3536 | −0.4695 | −0.5648 | 0.0630 | 0.0759 | −0.1339 |
| ARL3 | −0.0063 | −0.1409 | 0.0304 | −1.6033 | 0.3785 | 0.1219 | −0.0641 | −0.2895 |
| UBP1 | 0.0000 | −0.0780 | −0.1155 | −0.2065 | −0.0780 | 0.1513 | −0.1806 | 0.0902 |
| ATAD2 | 0.0073 | 0.1155 | −0.3219 | −0.2996 | −0.4594 | 0.2069 | −0.0473 | −0.0704 |
| AFTPH | −0.1866 | −0.0395 | −0.1520 | −0.4206 | −0.0589 | −0.0301 | 0.2451 | −0.2382 |
| ORMDL1 | −0.0418 | 0.0186 | −0.1520 | −0.4024 | −0.0780 | 0.3138 | 0.0000 | −0.1454 |
| MEA1 | −0.1188 | −0.0395 | −0.3219 | −0.7914 | −0.1240 | 0.0115 | 0.1699 | −0.2692 |
| SLC25A36 | −0.0919 | −0.6980 | −0.1926 | −0.5718 | 0.3943 | 0.0768 | −0.2594 | −0.0589 |
| RAET1E | −0.0371 | −0.0589 | −0.1155 | −0.8845 | −0.0671 | −0.0361 | 0.0000 | 0.0000 |
| DNAI2 | −0.0211 | −0.5475 | −0.1876 | −0.2216 | −0.0671 | −0.0181 | 0.1375 | 0.1375 |
| TEX26 | 0.1328 | −0.1155 | 0.0000 | −1.2065 | 0.0000 | 0.0000 | 0.0000 | 0.3785 |
| GTF2A2 | −0.0804 | −0.0395 | −0.1876 | −0.1325 | −0.2224 | −0.0348 | 0.4150 | −0.2065 |
| NIPAL3 | −0.0605 | −0.0199 | −0.2563 | −1.2996 | −0.1255 | 0.6553 | −0.0969 | −0.3785 |
| GFM2 | −0.1224 | −0.1699 | −0.2563 | −0.7105 | −0.0875 | −0.0301 | 0.0671 | 0.1926 |
| DIS3 | −0.0145 | 0.0186 | 0.0704 | −1.0000 | −0.2410 | 0.2849 | −0.1592 | 0.0202 |
| ERMP1 | 0.0000 | −0.0589 | 0.2224 | −0.5950 | 0.0444 | −0.1032 | −0.0217 | −0.0238 |
| EFHC2 | −0.0737 | 0.0000 | 0.0348 | −0.8105 | −0.0339 | 0.0000 | 0.0000 | 0.1375 |
| NME7 | −0.0071 | −0.1069 | 0.0931 | −1.0544 | −0.0995 | 0.2594 | −0.3785 | 0.0000 |
| ESRP1 | 0.0142 | −0.0721 | −0.2563 | −0.5399 | −0.0896 | 0.5477 | −0.1069 | −0.0896 |
| ADGRG1 | −0.0501 | −0.2730 | −0.0780 | −0.1133 | −0.3306 | 0.4155 | −0.1454 | −0.1409 |
| ST13 | −0.0780 | −0.1699 | 0.0000 | −0.8174 | −0.7370 | −0.3007 | −0.0544 | 0.0885 |
| SRGAP3 | 0.0395 | −0.2102 | −0.1876 | −0.1920 | −0.3219 | 0.4058 | −0.1592 | 0.2392 |
| C16orf71 | −0.0732 | −0.0780 | −0.1155 | −0.6539 | 0.0000 | 0.0000 | 0.0000 | 0.0000 |
| SPAG1 | 0.0630 | −0.3067 | −0.4739 | −0.5646 | −0.2977 | 0.1281 | 0.0220 | 0.1850 |
| HNRNPK | −0.1580 | −0.1155 | −0.0849 | −0.4415 | −0.2854 | 0.2594 | −0.6781 | −0.2065 |
| FAM104B | −0.0444 | −0.1155 | 0.1829 | −0.4695 | −0.1623 | 0.0895 | −0.0704 | −0.0704 |
| ABCA13 | 0.1699 | −0.1935 | −0.4150 | −0.7152 | −0.4800 | 0.1883 | −0.3219 | 0.4639 |
| CEP104 | −0.0501 | −0.2563 | 0.1829 | −0.5201 | −0.4657 | 0.1861 | 0.2630 | 0.1769 |
| LRRC34 | −0.0217 | −0.1876 | −0.0395 | −0.2471 | 0.0000 | 0.0000 | −0.0238 | −0.0473 |
| CCP110 | −0.0539 | −0.3759 | −0.0589 | −0.3764 | −0.0297 | 0.0212 | 0.1137 | 0.0931 |
| RBM19 | −0.0073 | −0.2692 | −0.1520 | −0.4024 | −0.4150 | 0.0971 | 0.0000 | 0.2630 |
| C8orf59 | −0.0804 | 0.0000 | −0.1155 | −0.5950 | −0.4150 | 0.2594 | 0.0566 | −0.1155 |
| NDUFB7 | −0.0986 | 0.0000 | −0.4448 | −0.4170 | −0.2515 | 0.8349 | 0.0489 | −0.2515 |
| EIF5 | −0.2983 | −0.5475 | −0.5236 | −0.1284 | 0.0369 | 0.7368 | 0.3741 | −0.9175 |
| ENKUR | 0.1858 | 0.0121 | −0.2895 | −1.3275 | 0.0000 | −0.0361 | −0.0238 | 0.2410 |
| CALM3 | 0.0000 | −0.0395 | 0.2630 | −0.6394 | −0.1592 | 0.3461 | 0.1561 | −0.4150 |
| ADNP | −0.0986 | 0.0759 | −0.2224 | 0.6680 | −0.1409 | 0.6173 | 0.1561 | −0.5525 |
| CDC5L | −0.1715 | −0.0141 | −0.2563 | −0.5915 | −1.1312 | 0.9018 | 0.0375 | −0.5688 |
| AC006064.4 | 0.0369 | 0.0000 | −0.3536 | −0.2065 | 0.0931 | −0.1881 | −0.3132 | 0.1137 |
| KRT23 | 0.0454 | −0.0969 | −0.1520 | 0.3930 | −0.4150 | 0.3849 | 0.4475 | −0.1065 |
| PRDX6 | 0.0304 | 0.0186 | −0.3219 | −0.3722 | −0.3219 | 0.2449 | 0.0704 | −0.3026 |
| KDM5C | −0.1388 | −0.1876 | −0.1155 | 0.2675 | −0.0431 | 0.1219 | 0.4352 | −0.5361 |
| GON7 | −0.1155 | −0.1339 | 0.0348 | −1.5475 | −0.1312 | 0.0030 | −0.0473 | −0.0931 |
| ZKSCAN1 | 0.0118 | −0.2546 | 0.1699 | −0.9841 | −0.3026 | 0.4566 | 0.5850 | −0.1775 |
| TDG | −0.0517 | −0.1339 | 0.1444 | −0.3320 | −0.1430 | 0.0971 | 0.1699 | −0.1155 |
| SLC25A5 | −0.1715 | −0.0395 | 0.1699 | −1.0940 | −0.2730 | 0.4114 | −0.1031 | −0.4792 |
| ZC2HC1A | −0.0211 | −0.0834 | −0.0395 | −1.1614 | −0.1312 | −0.1225 | 0.0444 | 0.2410 |
| SPAG16 | 0.0630 | −0.7549 | −0.5305 | −0.2471 | 0.0931 | −0.0625 | 0.0444 | −0.0238 |
| MMACHC | −0.0371 | 0.0000 | −0.3356 | −0.2996 | −0.1312 | 0.2088 | −0.1213 | −0.0238 |
| BEST1 | −0.0406 | −0.0199 | 0.0348 | 0.0451 | 0.1255 | 0.2594 | 0.0759 | −0.2224 |
| RNF19A | −0.2161 | −0.1876 | −0.0780 | 0.1769 | −0.6699 | 0.1903 | −0.1339 | 0.1026 |
| DYNC2H1 | 0.0397 | −0.0802 | 0.0740 | −0.1965 | −0.0875 | −0.1276 | −0.0931 | 0.2854 |
| CFAP36 | −0.0671 | −0.0780 | −0.2515 | −0.1857 | 0.1255 | 0.3208 | −0.0704 | 0.0902 |
| CLU | −0.2486 | 0.3806 | 0.0242 | −0.2324 | −0.1888 | 0.7988 | −0.4064 | 0.3699 |
| MT-ND4 | −0.1334 | −0.2206 | −0.0564 | −0.6852 | −0.1409 | −0.3380 | −0.0275 | 0.4970 |
| DUSP1 | −0.1449 | 0.0000 | −0.2224 | 0.9854 | 0.1076 | 0.3504 | −0.6907 | 0.0000 |
| AQP5 | 0.5670 | 0.3951 | −0.4150 | −0.5399 | −0.0109 | −0.3998 | 0.6485 | 0.0458 |
| RPL27 | 0.2295 | 0.0566 | −0.1993 | −1.2630 | −0.3692 | 1.2026 | 0.3956 | −0.5070 |
| DAAM1 | 0.2497 | −0.2086 | −0.0339 | 0.1155 | −0.4507 | 0.3644 | 0.0186 | −0.2224 |
| ATG3 | 0.0684 | −0.0395 | 0.2996 | −0.3440 | 0.1476 | −0.0762 | −0.0589 | 0.1255 |
| PKN2 | 0.1009 | 0.3870 | 0.2955 | −0.1894 | −0.7726 | −0.2746 | −0.0969 | 0.2996 |
| LCN2 | 1.0157 | 0.4406 | 0.0489 | −1.3955 | −0.0831 | −0.3927 | 1.0704 | 0.3346 |
| CCDC186 | 0.2039 | −0.0126 | −0.5850 | −0.0752 | −0.5384 | 0.9842 | 0.3923 | −0.8365 |
| KIF27 | 0.2134 | 0.1483 | −0.4448 | 0.1866 | 0.0825 | −0.0713 | 0.1137 | 0.2157 |
| NCL | 0.2572 | 0.2511 | −0.2630 | −0.3686 | −0.7667 | 0.2458 | 0.7578 | −0.7630 |
| ZNF141 | 0.0073 | 0.2439 | 0.0000 | 0.1826 | 0.0000 | 0.0030 | 0.0902 | 0.0000 |
| CFAP69 | 0.1959 | 0.0902 | −0.1876 | 0.0759 | 0.1255 | 0.0370 | 0.0000 | 0.0000 |
| BAZ1A | −0.0395 | 0.1699 | 0.0000 | 0.0831 | 0.9846 | 0.0923 | 0.1420 | −0.3641 |
| CD82 | 0.0067 | −0.0780 | −0.0671 | −0.6571 | −0.6374 | 0.6709 | 0.6001 | −0.0150 |
| TCEAL3 | 0.1069 | −0.0199 | −0.0395 | −0.0910 | 0.0614 | 0.3589 | −0.0931 | 0.0000 |
| ADGRF1 | 0.1402 | −0.1339 | 0.2955 | −1.0170 | −0.0911 | −0.2179 | 0.5850 | 1.1315 |
| PPARGC1A | 0.1143 | 0.0375 | 0.0000 | 1.0159 | 0.0000 | 0.0370 | 0.0902 | 0.0000 |
| BCAR1 | 0.1038 | −0.0780 | 0.2224 | 0.1435 | 0.0000 | −0.0011 | 0.4919 | −0.4150 |
| SERPINB1 | 0.3219 | 0.2025 | 0.1069 | −0.5950 | 0.6167 | 0.7791 | 0.0211 | −0.2801 |
| TES | −0.0349 | −0.0589 | −0.1155 | −0.1476 | 0.5343 | −0.0625 | 0.0304 | −0.3785 |
| SELENBP1 | 0.2233 | −0.2224 | 0.3626 | −1.0029 | −0.0184 | 0.2335 | 0.1444 | 0.3262 |
| CAPZA2 | −0.0280 | −0.0199 | 0.0000 | 0.3606 | 0.0825 | 0.4384 | −0.1520 | 0.0525 |
| PERP | 0.3237 | 0.0000 | −0.5025 | −0.8845 | −0.3435 | 1.0422 | −0.3833 | 0.0544 |
| MT-ND2 | 0.0557 | −0.4499 | −0.2582 | −0.4498 | −0.2662 | −0.0754 | 0.4624 | 0.5272 |
| CANX | 0.2738 | 0.5687 | 0.0000 | −0.4844 | −0.0682 | 0.6309 | −0.2443 | 0.1699 |
| MARVELD3 | 0.1149 | 0.3561 | −0.0780 | 0.4374 | 0.0406 | 0.3138 | −0.0931 | 0.2003 |
| LDLR | 0.1943 | −0.0780 | −0.5384 | −0.9642 | −0.9696 | 0.0971 | −0.1806 | 0.4150 |
| CAMLG | 0.0754 | −0.0199 | −0.0780 | −0.3320 | −0.2730 | 0.2453 | 0.3479 | 0.2630 |
| FASN | 0.0000 | 0.0566 | −0.3847 | 0.1895 | 0.2410 | 0.0262 | −0.0641 | 0.1926 |
| ANO10 | 0.0358 | −0.0199 | −0.0780 | −0.1069 | 0.0544 | 0.1281 | −0.0217 | 0.1476 |
| ARID4B | −0.1123 | 0.1829 | −0.3356 | 0.7056 | 0.6015 | 0.1625 | −0.1740 | 0.0719 |
| SLC26A2 | −0.0444 | 0.0956 | −0.1520 | −0.1857 | −0.7127 | 0.8367 | −0.2594 | 0.0671 |
| CNTRL | −0.0958 | 0.0980 | −0.4406 | 0.3081 | 0.0544 | 0.4561 | 0.3923 | 0.2630 |
| HECTD4 | 0.2696 | −0.3720 | −0.2224 | 0.1155 | 0.0825 | 0.4798 | −0.2429 | −0.0995 |
| MYL6 | −0.2194 | 0.2937 | −0.1255 | −1.1158 | −0.3923 | 0.3772 | 0.8381 | 0.1580 |
| GABRP | 0.0208 | 0.0956 | −0.3536 | −0.1476 | 0.5687 | 0.6744 | 0.3536 | 1.0424 |
| ATP5PD | −0.0406 | 0.4436 | 0.0000 | −0.1758 | −0.7370 | 0.8260 | −0.2076 | 0.2908 |
| PHB2 | 0.0358 | 0.1155 | −0.1155 | −0.2065 | −0.0641 | 0.0954 | 0.0885 | 0.0375 |
| ZFYVE21 | 0.0967 | −0.0395 | 0.0000 | 0.6236 | −0.3692 | 0.5664 | 0.4258 | 0.0406 |
| CD59 | −0.0649 | 0.0161 | 0.4150 | −1.3616 | −0.8556 | 0.0297 | 0.3196 | 0.1873 |
| CYTH1 | 0.1790 | 0.0577 | 0.2630 | −0.2065 | −0.0896 | −0.2711 | 0.3020 | 0.8194 |
| JMY | 0.1453 | 0.0525 | 0.1444 | 0.6130 | −0.5025 | 0.3927 | −0.0217 | 0.3237 |
| PEX16 | 0.1143 | 0.1155 | −0.0780 | 0.0831 | 0.3833 | 0.0895 | 0.2392 | 0.0444 |
| ID1 | 0.0754 | −0.0199 | −0.0395 | 0.1520 | 0.5392 | 0.3083 | 0.1489 | 0.2630 |
| COQ9 | 0.0489 | 0.0566 | −0.0395 | −0.0544 | 0.0544 | −0.0464 | 0.0825 | 0.0825 |
| HNRNPA1 | 0.0632 | 0.3561 | 0.1926 | −0.0220 | 0.1699 | 0.2199 | −0.0297 | −0.1339 |
| KIAA1468 | 0.1839 | 0.0525 | −0.1520 | −0.0544 | 0.2157 | 0.6744 | 0.1561 | −0.2594 |
| PLEKHM2 | 0.1291 | −0.0199 | −0.0780 | −0.1800 | −0.0525 | 0.2745 | 0.2410 | 0.5493 |
| CEP250 | 0.2768 | 0.1967 | 0.1069 | 0.1155 | −0.1430 | 0.1923 | −0.1375 | 0.1155 |
| ATP2B1 | 0.0067 | 0.0186 | −0.1520 | −0.6759 | 0.0406 | 0.4597 | 0.3626 | −0.1054 |
| NRBP1 | 0.1434 | −0.1155 | 0.2996 | −0.2926 | 0.0242 | 0.1636 | 0.5025 | 0.2295 |
| TNFRSF21 | 0.0802 | −0.0395 | 0.0704 | −0.2065 | 0.3421 | 0.3000 | 0.4150 | −0.0780 |
| ZNF226 | 0.1362 | 0.1255 | −0.0395 | 0.2310 | −0.2224 | 0.5395 | 0.0902 | −0.1155 |
| COX7B | 0.1470 | −0.1699 | −0.0589 | −0.8415 | 0.0000 | 0.6744 | 0.3100 | 0.1790 |
| HLA-E | 0.2151 | 0.1587 | 0.3370 | −1.9764 | −0.3904 | 0.8165 | 0.1646 | 0.0934 |
| NAA50 | 0.0597 | 0.0956 | −0.1520 | 0.1585 | 0.0000 | 0.3083 | 0.3923 | 0.1967 |
| TRMT44 | −0.0732 | −0.1339 | 0.0000 | 0.5305 | 0.2313 | 0.3762 | −0.0704 | 0.1444 |
| TNPO2 | −0.0071 | 0.0000 | −0.2563 | 0.4481 | 0.2895 | 0.3620 | 0.6063 | 0.1561 |
| TIMP2 | 0.2560 | 0.0000 | 0.0000 | 0.2895 | 0.3370 | 0.1697 | 0.1255 | 0.4944 |
| DDX60 | −0.0217 | −0.2065 | 0.1444 | 0.5305 | −0.1454 | 0.7386 | −0.3410 | −0.0931 |
| MLPH | 0.0847 | 0.0458 | 0.9279 | 0.3081 | −0.3219 | 0.7912 | −0.0506 | −0.2685 |
| RNF19B | 0.1578 | −0.0199 | 0.1829 | 0.4150 | 0.5611 | 0.0030 | 0.0444 | 0.1168 |
| BACE2 | 0.2630 | −0.0395 | 0.2955 | −0.2926 | −0.1651 | 0.4986 | 0.2462 | −0.1640 |
| ZBTB38 | 0.0614 | −0.0969 | −0.1155 | 0.6009 | −0.4300 | 0.1498 | 0.8480 | −0.0473 |
| ABCC1 | −0.0444 | 0.2630 | −0.0395 | −0.4695 | −0.1699 | 0.3849 | −0.0217 | 0.0902 |
| RNF213 | 0.2854 | −0.8931 | −0.1699 | 0.8524 | 0.5617 | −0.1138 | 0.2996 | −0.6037 |
| TSPAN15 | 0.0142 | −0.0199 | −0.0395 | −0.7105 | 0.0902 | 0.4939 | 0.1476 | 0.3251 |
| EPN2 | 0.0138 | −0.1240 | −0.1155 | −0.1325 | 0.6644 | 0.1513 | 0.1699 | 0.1769 |
| TJP3 | 0.0000 | −0.1775 | 0.1926 | −0.0681 | 0.6289 | −0.1466 | 0.1926 | 0.3039 |
| PCBP2 | 0.0599 | −0.1740 | −0.0671 | −0.2926 | 0.2494 | 0.5964 | 0.3862 | −0.1155 |
| CHCHD2 | 0.1155 | 0.3370 | −0.1155 | −0.9101 | −0.0217 | 0.7542 | 0.6181 | −0.2692 |
| AEBP2 | 0.0632 | 0.1520 | −0.0780 | 0.3081 | 0.1137 | 0.3927 | −0.1651 | 0.1829 |
| EIF3B | −0.0205 | 0.0000 | −0.0671 | −0.3440 | 0.2630 | 0.6804 | 0.6781 | 0.0186 |
| COMT | −0.0431 | −0.0395 | −0.0780 | 0.1155 | −0.2429 | 0.7136 | 0.2630 | −0.0849 |
| PUS1 | 0.0294 | −0.0199 | 0.1444 | 0.0451 | 0.2922 | −0.0136 | 0.0902 | −0.0238 |
| MXD1 | 0.0181 | −0.0199 | −0.1155 | 0.4236 | 0.1038 | 0.2755 | 0.2630 | 0.0839 |
| CSRNP1 | 0.0849 | 0.0000 | −0.0395 | 0.8524 | 0.1476 | −0.1056 | −0.0641 | −0.1054 |
| SENP6 | 0.0825 | −0.1375 | −0.0995 | 0.1155 | −0.3692 | 0.3730 | 0.0000 | −0.0184 |
| RACK1 | 0.2333 | −0.0896 | −0.1964 | −0.5972 | −0.3644 | 0.7099 | 0.7193 | −0.1520 |
| CLHC1 | 0.0725 | 0.3370 | 0.3755 | 0.1155 | −0.0671 | 0.5045 | 0.0000 | −0.0931 |
| HDAC9 | −0.0444 | 0.0956 | 0.3370 | 0.6301 | −0.1312 | 0.1074 | 0.3049 | −0.1651 |
| RNH1 | −0.0849 | −0.0199 | 0.0704 | −0.2400 | 0.0931 | 0.2121 | −0.0544 | 0.2330 |
| GDI2 | −0.0758 | 0.0956 | 0.2630 | −0.2996 | −0.1468 | 1.0249 | −0.0931 | −0.3999 |
| RHPN2 | −0.0431 | −0.0395 | 0.2224 | 0.1935 | 0.0740 | −0.0785 | 0.2630 | −0.1375 |
| IL13RA1 | 0.0214 | −0.0395 | 0.0704 | −0.4081 | 0.4245 | 0.8017 | 0.0375 | −0.2515 |
| SMAP2 | 0.1926 | 0.1026 | 0.4150 | 0.2854 | −0.5648 | 0.0223 | 0.1926 | −0.1454 |
| ZBTB4 | 0.0348 | −0.0589 | 0.3755 | −0.3060 | −0.0238 | 0.7680 | 0.1476 | 0.1155 |
| TPPP3 | 0.1884 | 0.0671 | −0.1155 | −1.8677 | −0.1312 | 0.4724 | 0.3923 | 0.7417 |
| SNAPC4 | −0.0071 | 0.0458 | −0.0671 | 0.4565 | −0.1623 | −0.0098 | 0.1255 | −0.1806 |
| CCDC173 | 0.0475 | 0.3923 | −0.4448 | 0.0451 | 0.0000 | −0.0181 | 0.0000 | 0.0000 |
| SAFB | 0.0338 | 0.1178 | −0.1926 | 0.3499 | 0.0375 | 0.0233 | 0.2838 | −0.0721 |
| HRASLS2 | −0.0371 | 0.1069 | −0.0395 | −0.1476 | 0.4525 | 0.0056 | 0.0000 | −0.0931 |
| GBP5 | −0.0875 | 0.0186 | −0.0395 | −0.0975 | 1.5208 | 0.0000 | 0.0000 | 0.0000 |
| SH3GLB1 | 0.0406 | −0.0184 | −0.1520 | 0.0649 | 0.4074 | 0.5713 | 0.4511 | −0.5081 |
| KHSRP | 0.0000 | 0.3020 | 0.4330 | −0.2996 | −0.2479 | 0.2461 | 0.0956 | 0.0614 |
| USP39 | 0.0358 | 0.0000 | 0.1444 | 0.4431 | −0.1592 | 0.3927 | 0.2193 | −0.0473 |
| ANKRD11 | 0.0740 | −0.1081 | −0.3458 | 1.2504 | 0.4361 | 0.0630 | 0.4312 | −0.3847 |
| CCNI | −0.0498 | 0.0956 | −0.4150 | −0.4363 | 0.0127 | 0.6825 | 0.7924 | 0.0348 |
| OTUD7B | −0.1361 | 0.3755 | −0.3219 | −0.3060 | −0.0473 | 0.6368 | 0.2630 | −0.2594 |
| IRF1 | 0.2370 | −0.0589 | 0.2274 | 0.0780 | −0.3169 | 0.7899 | −0.1069 | 0.2003 |
| CCDC6 | 0.0940 | −0.0969 | −0.1312 | 0.4374 | 0.4450 | 0.7569 | 0.1699 | −0.2730 |
| RAPGEFL1 | 0.0146 | −0.0395 | 0.0704 | −0.3955 | 0.7776 | 0.0835 | 0.6063 | −0.1375 |
| ZCRB1 | 0.0940 | 0.0759 | 0.2996 | −0.5519 | 0.1038 | 0.4090 | 0.2630 | −0.0641 |
| AUTS2 | −0.0205 | 0.1444 | −0.0339 | 0.6009 | −0.3026 | 0.5964 | 0.1155 | 0.2410 |
| SMIM5 | 0.0489 | 0.0000 | −0.0395 | 0.0330 | 0.1699 | 0.1185 | 0.2193 | 0.6350 |
| MAP1B | 0.0914 | 0.2091 | −0.0780 | −0.5139 | 0.0000 | 0.0718 | −0.0238 | 0.0000 |
| VRK2 | −0.0732 | 0.0566 | −0.1155 | 0.1435 | 0.1137 | 0.5687 | 0.1635 | −0.1454 |
| EIF6 | 0.1722 | −0.0589 | −0.2563 | −0.1325 | −0.1155 | 0.3613 | 0.4091 | 0.6092 |
| POLR2B | 0.0802 | 0.0000 | 0.2274 | 0.3081 | 0.5377 | 0.1116 | −0.2955 | 0.1699 |
| IFI16 | 0.1959 | 0.1967 | −0.4739 | 0.2202 | −0.0492 | 0.6825 | −0.1856 | −0.3668 |
| DOCK6 | 0.0614 | −0.0184 | −0.0395 | 0.2895 | 0.0000 | 0.4561 | 0.0902 | 0.2157 |
| TENT5A | 0.1909 | −0.0395 | 0.1926 | 1.1961 | −0.2496 | 0.5713 | −0.5850 | −0.3847 |
| DPM3 | 0.0277 | 0.0759 | −0.1155 | −0.5336 | 0.5261 | 0.4490 | 0.1155 | −0.2410 |
| BICDL1 | 0.0754 | 0.4056 | 0.0348 | 0.2086 | −0.4594 | 0.3959 | −0.0238 | 0.0825 |
| CEBPB | 0.1143 | −0.0199 | −0.0395 | 0.0831 | 0.6339 | −0.1831 | −0.0395 | 0.5261 |
| CRNN | 0.2364 | 0.0000 | 0.0000 | 0.1520 | 0.0000 | 0.0000 | 0.0000 | 1.1735 |
| CELSR1 | 0.1769 | −0.1430 | −0.1155 | −0.1216 | −0.3785 | 0.3265 | 0.2854 | 0.4919 |
| METAP2 | −0.0891 | 0.0348 | −0.1430 | 0.6618 | 0.1069 | 0.9037 | 0.4753 | −0.0896 |
| EPHA2 | −0.1089 | −0.1155 | −0.1155 | 1.3010 | 0.3312 | 0.3981 | 0.1635 | −0.6342 |
| FAM213A | −0.0945 | −0.0589 | 0.1444 | −0.6501 | −0.2977 | 0.2096 | −0.2410 | 0.2392 |
| S100A4 | −0.4455 | −0.0969 | 0.3833 | −0.7914 | −1.0251 | 0.9545 | 0.7639 | −0.8391 |
| MED15 | 0.0967 | −0.1155 | 0.2630 | 0.3930 | 0.4616 | 0.3849 | 0.3626 | −0.3306 |
| LRP8 | −0.0073 | 0.1069 | −0.0395 | −0.1614 | −0.0339 | −0.0538 | 0.2630 | 0.0000 |
| RPS15 | 0.2010 | 0.1255 | −0.2479 | −0.8565 | −0.0104 | 0.6575 | 0.4429 | −0.1946 |
| OAS3 | −0.0945 | −0.0589 | 0.0000 | −0.5239 | −0.5406 | 1.0647 | 0.0956 | 0.0375 |
| C2orf40 | 0.0871 | −0.1339 | 0.0000 | −0.2471 | 0.0000 | 0.1074 | −0.0473 | 0.1375 |
| KIAA1147 | 0.1328 | −0.0589 | −0.0780 | −0.4024 | 0.1864 | 0.7188 | 0.0444 | −0.1375 |
| TSPAN14 | 0.1186 | −0.0199 | −0.1876 | −0.3955 | 0.2099 | 1.0559 | 0.7335 | −0.0875 |
| HIVEP2 | −0.0661 | 0.2025 | 0.0000 | 0.4565 | 0.0489 | −0.2198 | 0.3755 | 0.1699 |
| ANXA5 | −0.0205 | −0.1155 | −0.3219 | −0.5738 | 0.2065 | 0.7289 | 0.4344 | 0.0902 |
| PLK2 | −0.0073 | 0.0000 | −0.0780 | 1.6301 | 0.8722 | −0.0817 | −0.6245 | −0.0159 |
| ZNF316 | −0.0073 | −0.0395 | 0.4695 | 0.0899 | 0.3219 | 0.3927 | 0.0000 | −0.1592 |
| TNNI3 | 0.0922 | −0.2934 | −0.1876 | 0.2310 | 0.0000 | 0.1255 | 0.2392 | 0.1375 |
| RABL2B | 0.0208 | 0.1255 | 0.2224 | −0.1476 | −0.1312 | 0.3298 | −0.0238 | −0.1592 |
| RBM17 | 0.2122 | −0.2065 | 0.0704 | −0.4170 | 0.3547 | 0.5785 | 0.5227 | 0.2824 |
| NTN1 | 0.0651 | −0.0199 | 0.2996 | −0.1800 | 0.0220 | 0.2461 | 0.6280 | 0.1137 |
| TNNI2 | 0.1871 | −0.0721 | 0.0000 | 0.2410 | 0.4420 | −0.1854 | 0.9588 | 0.0000 |
| CLASRP | −0.0145 | 0.2065 | −0.0395 | 0.1520 | 0.0544 | 0.2594 | 0.3312 | 0.1476 |
| YWHAE | 0.1196 | −0.0133 | −0.5525 | −0.9056 | −0.1520 | 0.9979 | −0.1676 | 0.4948 |
| GRM7 | 0.0220 | 0.3798 | 0.0000 | 0.0000 | −0.0339 | 0.0000 | 0.0000 | 0.1375 |
| ATP6V1G1 | 0.2900 | −0.1520 | 0.1699 | −1.1857 | 0.0867 | 0.8152 | 0.0465 | −0.2856 |
| EIF3E | 0.0214 | 0.0000 | 0.2996 | 0.2960 | 0.0740 | 0.4597 | 0.2439 | 0.0825 |
| CFH | 0.1118 | −0.0780 | 0.3833 | −0.1476 | 0.0121 | 0.7838 | −0.0834 | 0.3149 |
| ARRDC3 | −0.1024 | −0.0199 | −0.1520 | 1.2790 | 0.3455 | 0.3620 | −0.4475 | 0.1325 |
| CRYBG2 | 0.3892 | 0.0000 | 0.0000 | 0.7370 | 0.2630 | 0.0718 | 0.3312 | 0.0995 |
| PTP4A1 | −0.0395 | 0.0000 | 0.2274 | 0.5305 | −0.0395 | 0.6130 | −0.0732 | −0.0995 |
| TMPRSS11D | 0.4217 | 0.0000 | 0.0000 | 0.0000 | 0.0270 | 0.1525 | 0.1769 | 0.6634 |
| ZMIZ2 | 0.0208 | −0.2224 | −0.0395 | 0.2675 | 0.5552 | 0.3468 | 0.0000 | −0.0969 |
| SAR1B | −0.0071 | −0.0589 | −0.2224 | −0.1476 | −0.1758 | 0.4114 | 0.4630 | 0.1926 |
| TMEM231 | −0.0539 | 0.4381 | 0.1402 | −0.3570 | 0.0000 | 0.2049 | 0.0902 | 0.0000 |
| ARNTL2 | −0.0661 | 0.0000 | 0.1926 | 0.3499 | −0.2224 | 0.6829 | −0.0395 | 0.5984 |
| MYADM | −0.0073 | 0.0000 | 0.0348 | −0.0544 | 0.1110 | 0.6271 | 0.3951 | 0.0000 |
| TRAP1 | 0.1506 | 0.0304 | 0.2630 | 0.3379 | −0.1844 | −0.1913 | 0.4352 | 0.2025 |
| CHP1 | 0.1050 | −0.0199 | 0.0000 | −0.0055 | 0.1699 | 0.5224 | 0.0772 | −0.1043 |
| USP8 | 0.3322 | 0.1778 | 0.1255 | 0.4195 | −0.0995 | 0.6220 | 0.5110 | −0.0958 |
| HM13 | −0.0501 | −0.0780 | 0.2996 | 0.0610 | −0.3969 | 0.4731 | −0.0184 | 0.3755 |
| KPNA3 | 0.0475 | 0.1926 | 0.0000 | −0.0351 | 0.0220 | 0.0647 | 0.6431 | −0.2630 |
| SLC9A3R2 | −0.0517 | 0.0000 | 0.0000 | −0.1476 | 0.3119 | 0.6891 | 0.2392 | −0.2538 |
| IGF2BP2 | −0.0145 | 0.1402 | 0.0000 | 0.5894 | 0.1699 | 0.2594 | −0.1155 | 0.3699 |
| SLC37A1 | 0.0067 | −0.0506 | −0.1520 | 0.5811 | −0.1155 | 0.5224 | 0.5850 | 0.0406 |
| RPS24 | 0.0973 | −0.2801 | −0.0995 | −1.3521 | −0.1915 | 0.9758 | 0.7808 | −0.1375 |
| RASEF | 0.0802 | 0.2065 | 0.0704 | 0.5794 | 0.0000 | 0.1578 | 0.8946 | −0.3650 |
| SLC44A4 | 0.0807 | −0.3458 | −0.2801 | −1.5010 | −0.3626 | 0.8858 | 1.0336 | 0.5070 |
| IER3 | −0.0671 | 0.0956 | −0.1520 | −0.7396 | 0.5454 | 0.7289 | 0.3677 | −0.0525 |
| FOXA1 | −0.1541 | 0.0566 | 0.2274 | −0.6318 | −0.4011 | 0.4587 | 0.5110 | −0.0544 |
| PLCD3 | −0.0444 | 0.0000 | 0.0000 | 0.0000 | 0.2922 | 0.2594 | 0.8890 | 0.1850 |
| DEF8 | −0.0732 | 0.0375 | −0.0395 | 0.0280 | 0.4975 | 0.4014 | −0.0217 | 0.0614 |
| VCAN | 0.0146 | 0.0000 | −0.0780 | 0.2504 | 1.5850 | 0.0000 | −0.0238 | 0.0000 |
| 44450.0000 | −0.0289 | −0.1155 | 0.4150 | −0.2768 | 0.2630 | 0.1967 | −0.0473 | 0.1357 |
| STK36 | −0.0360 | 0.0458 | 0.1444 | 0.1155 | 0.0931 | 0.5568 | 0.0000 | 0.4616 |
| MINK1 | 0.0286 | −0.0969 | −0.0780 | −0.0975 | 0.0544 | 0.4293 | 0.0671 | 0.2224 |
| LMTK2 | 0.1561 | −0.0589 | −0.0780 | −0.3540 | −0.1699 | 0.8735 | 0.6674 | 0.3340 |
| GBF1 | 0.0754 | −0.2895 | 0.2996 | 0.0566 | 0.3370 | 0.0098 | −0.1375 | 0.5025 |
| POMT2 | 0.0849 | −0.0184 | −0.0395 | 0.0451 | −0.1312 | −0.0361 | −0.0704 | 0.0000 |
| MT-ATP8 | −0.2419 | −0.5381 | −0.4349 | 0.1847 | −0.3440 | −0.0073 | 0.4718 | 1.0774 |
| ZBED1 | −0.0145 | −0.0969 | −0.0395 | −0.2630 | 0.2630 | 0.3686 | 0.3699 | 0.4352 |
| MCU | −0.0555 | 0.1635 | −0.2224 | 0.7004 | −0.2035 | −0.1695 | 0.5146 | 0.2922 |
| SYT5 | −0.0732 | 0.1444 | −0.0395 | −0.0170 | 0.0000 | 0.0000 | 0.1375 | −0.0238 |
| ITGA6 | −0.0371 | 0.1829 | 0.4150 | −0.0544 | 0.0000 | −0.0136 | 0.5539 | −0.1054 |
| RSL1D1 | 0.0802 | −0.0721 | 0.1255 | −0.2471 | −0.4681 | 0.7463 | 0.4361 | −0.3026 |
| ABHD11 | 0.1149 | 0.0161 | 0.3370 | −0.1894 | 0.1255 | −0.1067 | 0.4764 | −0.0589 |
| BMPR1B | 0.0651 | −0.0199 | 0.1829 | 0.3081 | 0.1402 | 0.2736 | −0.0365 | −0.0704 |
| FGFBP1 | 0.1293 | 0.0000 | 0.3755 | 0.0000 | −0.0995 | 0.3319 | 0.1881 | −0.0931 |
| RASSF5 | 0.0208 | −0.0395 | 0.2224 | −0.0101 | −0.2224 | −0.0464 | 0.3312 | 0.9069 |
| TCN1 | −0.0075 | 0.0000 | 0.0000 | 0.0000 | −0.2801 | 1.4014 | 0.7875 | 0.0671 |
| SDC4 | −0.0365 | 0.0186 | 0.4330 | −0.4277 | −0.4250 | 0.8645 | 0.4819 | −0.2224 |
| SDCBP2 | 0.2490 | −0.0199 | −0.1520 | 0.1826 | −0.4330 | 0.3849 | 0.3798 | 0.1226 |
| PER3 | −0.0517 | 0.0186 | −0.0395 | 0.8524 | 0.1255 | 0.2594 | −0.0395 | −0.1155 |
| NUCKS1 | 0.0186 | 0.0725 | 0.1255 | −0.4106 | −0.1520 | 0.7109 | 0.3070 | −0.4330 |
| SLC16A9 | 0.0776 | 0.0956 | −0.0395 | −0.1614 | −0.2016 | 0.7246 | 0.2630 | −0.0704 |
| MRPS26 | −0.0371 | −0.0589 | 0.5070 | −0.2768 | 0.2854 | 0.3278 | 0.4764 | −0.2829 |
| IFIT3 | 0.0369 | −0.0969 | 0.2224 | −0.2065 | −0.5443 | 0.5749 | −0.0365 | 0.8194 |
| CHD1 | 0.1894 | 0.3991 | −0.3536 | 0.6267 | −0.1240 | 0.6387 | 0.1881 | 0.3326 |
| LRRC8A | −0.0732 | −0.0199 | −0.0780 | 0.4481 | 0.1561 | 0.3138 | 0.2937 | 0.1019 |
| HERC5 | −0.0150 | 0.0000 | −0.0780 | 0.7232 | 1.3093 | −0.0361 | 0.0000 | −0.1155 |
| SWAP70 | 0.1186 | −0.0969 | 0.0348 | 0.2410 | −0.2895 | 0.0030 | −0.0995 | 0.1255 |
| TMEM106B | 0.1481 | 0.0000 | −0.0780 | 0.1895 | 0.7521 | 0.1365 | −0.0506 | 0.5850 |
| YARS | 0.0000 | 0.0566 | 0.2996 | 0.0610 | 0.6339 | 0.4709 | 0.0406 | 0.2630 |
| N4BP1 | 0.1186 | −0.0395 | −0.1155 | 0.0899 | 0.0186 | 0.9268 | −0.2630 | 0.0825 |
| ALDH3A1 | −0.9239 | −0.1946 | −0.5619 | 0.5402 | −0.3788 | 1.3379 | 1.2299 | −0.8875 |
| TBC1D9B | 0.2765 | −0.0969 | 0.0704 | −0.2996 | 0.4150 | 0.2270 | 0.3219 | −0.1081 |
| TMPRSS2 | 0.1255 | 0.1926 | 0.2274 | −0.7720 | 0.7454 | 0.2277 | 0.2025 | 0.6702 |
| TRIP10 | 0.1118 | −0.0395 | 0.1444 | −0.2996 | 0.5850 | 0.2728 | 0.2439 | 0.4056 |
| MEPCE | 0.0995 | −0.0199 | 0.2274 | 0.3606 | −0.1278 | 0.3686 | −0.0431 | 0.1769 |
| AKAP13 | −0.0365 | 0.0584 | 0.0348 | 0.4102 | −0.0864 | 0.3525 | 0.8713 | −0.2313 |
| PEA15 | −0.0986 | −0.0199 | 0.4695 | −0.2768 | −0.4150 | 0.3561 | 0.1343 | 0.8316 |
| PELP1 | −0.0589 | 0.1069 | 0.1829 | −0.3320 | 0.1520 | 0.5531 | 0.2157 | −0.0473 |
| EAPP | 0.2630 | 0.0000 | 0.2996 | −0.4695 | −0.6951 | 1.0041 | 0.1561 | 0.0614 |
| ITPR1 | 0.0560 | 0.2812 | −0.0395 | 0.0280 | 0.2922 | −0.0625 | −0.0238 | −0.0238 |
| DIO2 | −0.0297 | 0.2224 | 0.0000 | 0.0000 | −0.0339 | 0.1074 | 0.0000 | 0.7776 |
| LSM8 | 0.0146 | 0.0956 | 0.1444 | −0.5475 | 0.2895 | 0.8883 | −0.1592 | 0.0202 |
| IRS2 | 0.0614 | −0.0199 | 0.0000 | 0.4310 | −0.0159 | 0.4610 | 0.5651 | 0.4150 |
| FXYD3 | 0.1014 | 0.0525 | −0.0339 | −1.1393 | −0.2895 | 0.2403 | 0.4603 | 0.1609 |
| TM9SF2 | 0.2364 | 0.0566 | 0.3973 | −0.3764 | −0.2801 | 0.5328 | 0.0286 | −0.1375 |
| CAPN5 | −0.0431 | −0.0199 | −0.0395 | −0.3440 | 0.2193 | 0.6173 | 0.4150 | 0.3455 |
| LETM1 | −0.0589 | 0.2439 | 0.2630 | 0.4481 | −0.3744 | 0.1941 | 0.4764 | 0.1444 |
| DDX24 | 0.0000 | −0.2370 | −0.1926 | 0.3129 | −0.2659 | 0.3000 | 0.3973 | −0.0589 |
| RSBN1L | −0.0067 | 0.0161 | 0.5208 | 0.3379 | −0.4695 | 0.5128 | 0.5025 | −0.2051 |
| SUPT6H | 0.0825 | −0.0834 | −0.0395 | 0.4634 | −0.6374 | 0.2594 | 0.5639 | 0.2099 |
| PKP3 | 0.0000 | −0.0199 | −0.0395 | −0.1325 | 0.1375 | 0.7241 | 1.0995 | −0.1740 |
| RARRES2 | 0.1434 | 0.0000 | 0.0000 | 0.2086 | −0.0589 | 1.1525 | 0.3312 | 0.1375 |
| DUS1L | 0.0462 | −0.1876 | 0.3755 | −0.6270 | 0.0000 | 0.3183 | 0.4150 | 0.2824 |
| C9orf78 | 0.0825 | 0.0000 | −0.3219 | −0.1216 | 0.0406 | 0.3787 | 0.4475 | 0.1520 |
| MDM4 | −0.0289 | 0.1806 | 0.3286 | −0.2580 | 0.0614 | 0.5356 | 0.1979 | −0.1844 |
| EPC1 | 0.0348 | −0.1339 | −0.1155 | 0.6598 | −0.2479 | 0.4610 | −0.0395 | −0.1240 |
| HNRNPA2B1 | 0.0937 | 0.0931 | 0.0324 | −0.8356 | −0.1907 | 0.6502 | −0.3467 | −0.4739 |
| CTSS | −0.2912 | 0.5178 | 0.3370 | −0.9201 | −0.0780 | 0.2242 | 0.1747 | 0.1019 |
| MROH1 | 0.0073 | 0.0995 | 0.1069 | −0.1216 | 0.5040 | −0.1417 | 0.1155 | −0.0544 |
| IL6R | −0.0224 | −0.0199 | −0.0395 | 0.2257 | 0.1699 | 0.3083 | −0.0704 | −0.0473 |
| CGN | 0.1612 | −0.3219 | −0.1876 | −0.1740 | 0.7335 | 0.3018 | 0.9661 | −0.3388 |
| SQSTM1 | 0.3128 | 0.2630 | −0.3847 | 0.4780 | −0.7242 | 0.5081 | −0.0525 | −0.3830 |
| SLC39A7 | −0.0205 | −0.0780 | 0.1069 | −0.3175 | −0.1015 | 0.7178 | 0.2630 | 0.4579 |
| GNAS | −0.0135 | −0.2515 | 0.2895 | −0.3440 | −0.6292 | 0.7977 | 0.8360 | 0.1216 |
| GPRC5A | 0.2500 | −0.0589 | 0.0704 | 0.3081 | −0.1940 | 0.6685 | 0.8086 | 0.1627 |
| FUS | −0.1155 | 0.0577 | −0.4594 | 1.0610 | 0.1312 | 0.8859 | 0.2762 | −0.2730 |
| CCDC66 | 0.1605 | 0.0465 | −0.0589 | 0.9175 | 0.5552 | 0.0030 | −0.0704 | 0.0671 |
| RAB14 | 0.0544 | 0.0759 | 0.1069 | 0.1679 | 0.1756 | 0.6335 | 0.3455 | −0.3155 |
| ACTB | −0.0199 | −0.0365 | −0.0875 | −0.3090 | −0.2515 | 0.6668 | −0.1387 | 0.2740 |
| FAM219B | 0.1362 | 0.0000 | 0.1069 | −0.2768 | −0.2016 | 0.2314 | 0.4919 | 0.0186 |
| DUOX1 | 0.1022 | 0.3370 | −0.3847 | 0.0759 | −0.1775 | 0.1646 | 0.0956 | 0.6630 |
| DNAJC3 | −0.0205 | 0.1444 | −0.0671 | −0.3820 | 0.0614 | 0.5628 | 0.2182 | 0.7004 |
| IRF2BPL | 0.0071 | −0.0395 | 0.1829 | 0.2960 | 0.0995 | −0.1417 | 0.0902 | −0.0238 |
| ADIRE | −0.0418 | 0.0000 | −0.0780 | −0.0544 | −0.2605 | 0.9770 | 0.7705 | 0.7847 |
| SLC7A11 | 0.0725 | −0.0395 | 0.1444 | 1.4780 | −0.0995 | 0.0718 | −0.0704 | 0.0566 |
| ACBD3 | 0.1578 | 0.4881 | 0.0000 | 0.1895 | −0.2382 | 0.6938 | 0.4066 | 0.0134 |
| CBX3 | 0.4044 | 0.0995 | −0.1623 | −0.2926 | −0.0896 | 0.4372 | −0.0875 | −0.1806 |
| CD24 | −0.0854 | −0.0721 | 0.0544 | −1.0197 | 0.3991 | 0.4853 | 0.7210 | 0.6234 |
| FAF2 | 0.0431 | −0.1155 | −0.1155 | 1.1155 | −0.2451 | 0.4939 | 0.3262 | 0.5661 |
| DDIT4 | −0.1964 | −0.1699 | 0.2274 | 0.6324 | −0.3585 | 0.1599 | 0.1255 | −0.1876 |
| IQCB1 | 0.0825 | 0.6431 | 0.0348 | −0.0825 | 0.3370 | 0.2594 | 0.1926 | 0.0902 |
| SNX9 | 0.1328 | 0.2025 | −0.0780 | 0.2086 | 0.1864 | 0.5377 | 0.0186 | 0.4406 |
| ST8SIA4 | −0.0224 | −0.0199 | −0.1520 | 0.0159 | −0.3923 | 0.7740 | 0.7182 | 0.1375 |
| MEF2A | −0.0141 | −0.0896 | −0.1876 | 0.4634 | 0.4475 | 0.1719 | 0.2439 | 0.2099 |
| WDR90 | −0.0272 | −0.0444 | −0.3219 | 0.8716 | −0.1430 | 0.0212 | 0.1038 | 0.5626 |
| RIOK3 | 0.0416 | 0.0656 | 0.1926 | 0.9456 | −0.1278 | 0.5109 | −0.0506 | −0.4854 |
| PPARGC1B | −0.0297 | −0.0199 | −0.0395 | 0.4236 | 0.7370 | 0.2767 | 0.4381 | −0.0238 |
| RPS13 | −0.0120 | 0.2251 | −0.2224 | −0.4024 | −0.0265 | 0.7622 | 0.4102 | −0.1089 |
| CBX6 | 0.0220 | −0.0780 | 0.7965 | −0.4695 | −0.4150 | 0.3508 | 0.3973 | 0.0220 |
| MTRNR2L6 | 0.1190 | 0.0875 | −0.1520 | 0.1155 | −0.0265 | 0.4724 | −0.1520 | −0.0589 |
| GALNT12 | −0.0217 | −0.0199 | 0.4150 | −0.3699 | −0.1339 | 0.6106 | 0.2099 | 0.1375 |
| NARF | −0.1565 | −0.0896 | 0.1444 | −0.3440 | 0.6845 | 0.3025 | 0.1255 | −0.3890 |
| PALLD | −0.0063 | −0.0297 | −0.2563 | −0.1598 | 0.1520 | 0.1245 | 0.7510 | 0.3119 |
| RPS15A | 0.2698 | −0.0395 | 0.1699 | −0.6394 | 0.1343 | 0.8981 | 0.0614 | 0.1967 |
| EMP1 | 0.3644 | 0.0000 | 0.3755 | 0.0000 | 0.4764 | −0.0885 | −0.6738 | 0.3651 |
| SMAD4 | −0.0289 | 0.1635 | 0.0000 | 0.4525 | 0.2630 | 0.2745 | 0.4764 | −0.0217 |
| SELENOF | 0.1118 | −0.0395 | 0.0000 | −0.5626 | 0.0489 | 0.5895 | 0.0704 | 0.4639 |
| PSME3 | 0.0754 | 0.1635 | −0.1155 | −0.4695 | 0.9125 | 0.5369 | 0.0566 | −0.3132 |
| TMEM160 | 0.0202 | −0.0199 | −0.0995 | −0.1800 | −0.2801 | 0.6891 | 1.1468 | 0.0489 |
| UGCG | 0.0329 | −0.0589 | 0.2630 | 0.3701 | −0.2224 | 0.6220 | 0.3923 | 0.4220 |
| ZNF397 | 0.0286 | 0.0186 | 0.4695 | 0.3499 | 0.5850 | 0.0818 | −0.0473 | −0.1155 |
| FAM177A1 | −0.0431 | 0.0704 | −0.1155 | 0.1520 | 0.0525 | 0.3981 | 0.0759 | 0.3951 |
| SLU7 | 0.0733 | 0.0151 | −0.2895 | −0.1476 | 0.2224 | 1.0738 | 0.4436 | −0.2942 |
| RPL27A | 0.0332 | −0.0544 | 0.2873 | −1.1822 | −0.0973 | 0.9882 | 1.0144 | −0.8817 |
| RHOC | 0.0825 | 0.2224 | 0.0348 | −0.0170 | 0.2157 | 0.3608 | 0.6153 | 0.0000 |
| UBE2E1 | 0.0071 | 0.0759 | 0.0704 | −0.1325 | 0.2345 | 0.4144 | 0.5189 | 0.0406 |
| RAB5IF | 0.0632 | 0.0000 | 0.0000 | 0.2675 | −0.3026 | 0.4798 | −0.0834 | 0.5850 |
| MYOF | 0.0356 | −0.2382 | −0.0995 | 0.4403 | 0.0462 | 0.0306 | 0.5850 | 0.1520 |
| RPS4Y1 | −0.0945 | −0.1155 | −0.2224 | −0.7496 | −0.0671 | 0.5224 | 0.2801 | 0.0406 |
| TUBA1C | 0.0000 | −0.0199 | 0.0000 | −0.3440 | −0.1699 | 0.8560 | 0.5406 | −0.0431 |
| RAB9A | 0.1291 | 0.0000 | 0.0000 | 0.2086 | 0.4297 | 0.4400 | −0.0431 | 0.6855 |
| OGT | −0.0205 | 0.0000 | 0.0348 | 0.1826 | 0.2115 | 0.3849 | 0.4975 | 0.3833 |
| EIF5A | 0.1050 | −0.0199 | 0.1255 | −0.3060 | 0.1069 | −0.0224 | 0.6974 | 0.2251 |
| ZBTB7A | 0.0000 | −0.0171 | 0.1444 | 0.1826 | −0.7238 | 0.7691 | 1.2531 | 0.5734 |
| GNB2 | 0.1464 | −0.0199 | −0.1155 | −0.0825 | 0.3479 | 0.6685 | 0.7036 | −0.0704 |
| TNFRSF12A | −0.0224 | −0.0199 | −0.1155 | 0.9711 | −0.2224 | 0.6744 | 1.1069 | 0.0000 |
| PHF3 | 0.1677 | 0.3191 | 0.0614 | 0.0159 | 0.2224 | 0.3418 | −0.2224 | 0.4445 |
| PDZD8 | 0.0208 | 0.0000 | −0.1876 | −0.5850 | 0.1343 | 0.6379 | 0.2274 | 0.0614 |
| OAS1 | −0.0137 | −0.0199 | −0.1155 | −0.4695 | −0.5090 | 0.7048 | 0.5454 | 0.8445 |
| SPRR2D | 0.4975 | 0.0000 | 0.0000 | 0.0000 | 0.0000 | −0.0538 | 0.0000 | 1.0579 |
| RPS21 | 0.2106 | −0.1339 | 0.2370 | −1.4950 | −0.6439 | 1.3496 | 1.2238 | −0.2016 |
| TYMP | 0.4215 | −0.2382 | −0.2895 | 0.1087 | 0.5619 | 0.1391 | 0.5670 | −0.6590 |
| MPC1 | 0.0069 | 0.0186 | 0.4695 | 0.4150 | −0.0931 | 0.4334 | −0.3785 | 0.1881 |
| SLC4A1AP | 0.2108 | 0.2251 | 0.0348 | 0.2086 | 0.1110 | 0.0691 | 0.4695 | −0.1699 |
| EPS8L2 | −0.0258 | 0.1333 | 0.1587 | −0.6789 | 0.1959 | 0.2410 | 1.0458 | −0.1520 |
| PPP4R2 | 0.0358 | 0.0375 | 0.1069 | 0.0566 | 0.1476 | 0.4165 | 0.1444 | 0.1756 |
| TOP1 | 0.0867 | −0.1468 | 0.1255 | 0.6725 | 0.5291 | 0.1642 | 0.4235 | 0.2709 |
| MAN2C1 | −0.0297 | −0.0969 | −0.1155 | −0.2350 | 0.4780 | 0.1663 | 0.3081 | 0.0406 |
| KLK13 | −0.0224 | 0.0000 | 0.0000 | 0.0000 | 0.0000 | 0.1074 | 0.0000 | 1.7521 |
| UBR4 | 0.0983 | −0.0958 | −0.1520 | 1.1155 | −0.4313 | 0.0370 | 0.8745 | 0.4074 |
| CABIN1 | −0.0280 | −0.0978 | −0.2895 | 0.8842 | 0.2065 | 0.1185 | −0.1155 | 0.0614 |
| UBE3A | 0.1464 | −0.0184 | −0.1876 | −0.4206 | 0.0000 | 0.6689 | 0.2065 | 0.0173 |
| CCPG1 | 0.1033 | −0.1640 | 0.1255 | −0.4933 | −0.1915 | 1.0184 | 0.0875 | 0.2500 |
| TMBIM1 | −0.1059 | 0.0000 | −0.0395 | 0.3081 | −0.1651 | 0.6668 | 0.6656 | 0.2274 |
| RPL36 | 0.0000 | 0.0759 | 0.0825 | −0.8845 | −0.2382 | 1.3160 | 0.7190 | −0.1155 |
| COX6B1 | 0.1223 | 0.0704 | −0.1623 | −0.9495 | 0.1019 | 0.9108 | 0.4944 | 0.0390 |
| CLOCK | −0.0501 | 0.1444 | −0.1520 | 0.3930 | 0.5850 | 0.9375 | 0.2274 | 0.2099 |
| DDX60L | 0.1328 | 0.1092 | 0.2224 | 0.1435 | 0.1979 | 0.0212 | −0.0849 | 0.0614 |
| SEL1L | −0.0360 | 0.2824 | −0.1520 | −0.0544 | −0.2675 | 1.0172 | 0.0525 | 0.3755 |
| UNC93B1 | 0.1291 | −0.1240 | −0.2563 | 0.1730 | 0.3862 | 1.2950 | 0.0186 | −0.2016 |
| KRT18 | 0.2515 | −0.1468 | 0.4898 | −0.5775 | 0.0060 | 0.7315 | 0.5025 | 0.3403 |
| MTDH | −0.0460 | 0.1926 | −0.1926 | 0.0999 | −0.0614 | 1.0011 | 0.4898 | 0.1646 |
| TXNIP | 0.1536 | 0.3244 | −0.1430 | 1.3969 | −0.1887 | 0.2910 | −0.0882 | −0.2998 |
| F2RL1 | 0.0577 | −0.0199 | 0.1444 | −0.0101 | 0.0995 | 0.7448 | 0.1155 | 0.4616 |
| ARHGDIA | 0.0000 | 0.0000 | −0.0780 | −0.4081 | 0.4420 | 0.0930 | 0.6630 | 0.3833 |
| MT2A | 0.1255 | 0.0375 | 0.1402 | −0.2926 | −0.3890 | 0.9798 | −0.0395 | 0.3785 |
| EBP | 0.0286 | 0.0000 | 0.1444 | 0.2310 | −0.3692 | 0.5502 | −0.1339 | 0.1829 |
| CIR1 | −0.0349 | 0.2955 | −0.1623 | −0.4288 | 0.1561 | 0.3038 | 0.4919 | 0.6041 |
| CLK2 | 0.0073 | −0.0969 | 0.0704 | 0.1895 | 0.0544 | 0.1967 | 0.1137 | 0.2630 |
| KLF4 | −0.0525 | −0.0395 | −0.2224 | 0.9264 | −0.2801 | 0.8765 | −0.3356 | 0.1890 |
| AQP3 | 0.0965 | −0.3219 | 0.2873 | −0.5959 | −0.5291 | 1.1298 | −0.0223 | 0.1125 |
| FDPS | −0.0069 | −0.0589 | 0.4150 | −0.6539 | −0.0395 | 0.1814 | 0.2955 | 0.4406 |
| KLF5 | −0.1829 | −0.0969 | −0.1592 | −0.6124 | 0.4175 | 1.4334 | 0.5661 | −0.3119 |
| CEACAM1 | 0.0544 | −0.0199 | 0.0000 | 0.0000 | 0.0000 | −0.0301 | 0.8480 | 0.9941 |
| PRKAR1A | −0.2005 | 0.1587 | −0.0339 | −0.4866 | 0.5387 | 0.3313 | −0.2076 | 0.4406 |
| SLK | 0.1666 | −0.2651 | −0.1430 | 0.2086 | −0.1993 | 1.3780 | 1.5560 | 0.0329 |
| DNAJC15 | −0.0217 | −0.0589 | −0.0671 | −0.1857 | −0.2035 | 0.7740 | 0.7406 | −0.1255 |
| SPATS2L | 0.0065 | −0.0825 | −0.0875 | −0.3802 | −0.1430 | 1.1938 | 0.6590 | −0.0444 |
| LPCAT4 | −0.0280 | 0.1829 | −0.0995 | −0.0170 | −0.1651 | 0.6537 | 0.2824 | 0.4695 |
| CHD2 | 0.0500 | 0.1599 | 0.2274 | 1.5114 | 0.2630 | 0.7802 | 0.0173 | −0.5934 |
| B3GNT5 | 0.0065 | 0.1168 | −0.1155 | 0.4930 | 0.2630 | −0.0136 | 0.0220 | 0.4695 |
| RABL6 | 0.0202 | 0.0000 | −0.0589 | 0.0097 | 0.2462 | 0.3693 | 0.8417 | 0.4910 |
| KTN1 | 0.0975 | −0.0671 | −0.2730 | −0.8668 | −0.5070 | 0.7802 | 0.4874 | 0.0242 |
| PSMD3 | 0.0614 | 0.0375 | 0.2630 | 0.0610 | −0.2977 | 0.8809 | 0.3421 | 0.1881 |
| CD63 | −0.0404 | 0.0704 | −0.3692 | −1.2471 | −0.5400 | 1.4130 | −0.2872 | 0.0938 |
| GORASP2 | 0.0214 | −0.0395 | 0.3755 | 0.2310 | 0.4420 | 0.4553 | 0.3020 | 0.1881 |
| PER2 | −0.0071 | −0.0395 | −0.2895 | 1.5987 | −0.3626 | 0.8756 | 0.0186 | 0.0406 |
| RPP38 | −0.0289 | −0.0171 | 0.1829 | 0.0000 | −0.2224 | 0.2755 | 0.1979 | −0.1054 |
| SYAP1 | 0.0660 | 0.1587 | 0.2630 | −1.2996 | −0.0896 | 0.6630 | 0.2274 | 0.5122 |
| FUBP1 | 0.0871 | 0.3049 | −0.0671 | 0.2895 | −0.1844 | 0.4069 | 0.0956 | 0.1926 |
| STARD10 | −0.0069 | 0.0000 | 0.3370 | 0.1679 | −0.5502 | 0.4740 | 0.4056 | 0.2025 |
| HES1 | 0.0450 | 0.0186 | −0.1278 | 2.0765 | −0.5949 | 0.6960 | −1.1240 | −0.6579 |
| MYCBP2 | −0.0199 | −0.0671 | 0.1926 | 0.3190 | 0.6554 | 0.1625 | 0.1587 | −0.0159 |
| FEM1A | −0.0555 | −0.0395 | −0.0395 | 0.0000 | 0.5850 | 0.3525 | 0.3312 | 0.1038 |
| EEA1 | −0.0069 | −0.1775 | 0.0348 | −0.2285 | 0.0566 | 0.3265 | 1.1357 | −0.0395 |
| RSRC2 | −0.0059 | −0.3458 | 0.0242 | 1.1826 | −0.6477 | 0.6427 | 0.6897 | 0.1155 |
| SLC12A6 | 0.3049 | −0.1155 | 0.0000 | −0.1325 | 1.0000 | 0.3772 | 0.3626 | 0.2025 |
| RPL37A | 0.1812 | −0.0473 | 0.0614 | −1.4695 | −0.4999 | 1.2150 | 1.0896 | −0.1822 |
| RBM3 | −0.0448 | 0.0956 | −0.2730 | 0.7715 | −0.4525 | 1.0821 | 0.7995 | 0.1756 |
| ABHD2 | 0.0746 | −0.0365 | 0.4297 | −0.4509 | −0.6781 | 0.6155 | 1.1155 | 0.0161 |
| PDCD11 | −0.0875 | −0.1664 | 0.1587 | 0.7784 | 0.2895 | 0.6033 | 0.3699 | 0.1255 |
| SPINT1 | 0.0406 | −0.0780 | −0.0780 | 0.2086 | 0.1420 | 0.4548 | 0.7162 | 0.2320 |
| TFCP2L1 | −0.0349 | 0.1444 | −0.1312 | 0.5305 | 0.4461 | 0.8315 | −0.2845 | −0.1155 |
| DNM2 | 0.0544 | −0.0544 | 0.1069 | −0.6539 | 0.2630 | 0.5224 | 0.2224 | 0.0825 |
| BSPRY | −0.0945 | 0.0000 | 0.2996 | −0.0544 | 0.0759 | 0.7634 | 0.8546 | 0.0614 |
| IFIT2 | −0.0517 | −0.0199 | 0.0000 | 0.8524 | 3.2390 | 0.0971 | 0.0220 | −0.0238 |
| PLSCR1 | 0.0338 | 0.1635 | −0.1155 | 0.3861 | −0.1155 | 0.3730 | 0.0000 | 0.3272 |
| DUSP5 | 0.0329 | 0.0000 | −0.1155 | 0.7370 | −0.2675 | 0.0718 | 0.6868 | 0.8391 |
| METTL7A | −0.2168 | −0.2730 | −0.1520 | −1.3374 | −0.2065 | 0.4766 | 0.0000 | 0.4381 |
| ALDH1A1 | −0.0751 | 0.1178 | 0.4095 | −0.8460 | −0.3431 | 0.2674 | 0.5155 | 0.2091 |
| TACC2 | 0.1155 | −0.1699 | 0.4806 | 0.9090 | 0.2520 | 0.7035 | 0.5070 | 0.4975 |
| BEST4 | −0.0211 | 0.3959 | 0.2224 | −0.2768 | −0.0339 | 0.1255 | 0.1375 | 0.2630 |
| MVP | 0.0067 | 0.0173 | 0.1587 | 0.0869 | −0.4150 | 0.6134 | 0.4695 | −0.4525 |
| B4GALT4 | 0.1186 | 0.0956 | −0.1520 | 0.0566 | 0.0671 | 0.7680 | 0.6831 | 0.0525 |
| SF3B2 | −0.0721 | 0.1375 | −0.2224 | −0.3440 | −0.1565 | 0.5813 | 0.7335 | −0.0498 |
| FOXJ1 | 0.0131 | 0.3626 | −0.0704 | −0.1800 | −0.0671 | 0.1074 | −0.0238 | 0.2630 |
| LGALS3BP | −0.0141 | 0.0885 | −0.0671 | 0.1300 | −0.4349 | 0.8322 | 0.3119 | 0.0444 |
| RPL11 | 0.2350 | 0.3119 | −0.0931 | −1.5399 | −0.1964 | 0.6184 | 1.1907 | −0.2035 |
| REEP3 | −0.0501 | −0.0589 | 0.4150 | −0.2350 | −0.0184 | 1.0559 | 0.3774 | −0.2361 |
| LY6E | 0.0825 | 0.0759 | 0.0000 | −0.3362 | −0.4930 | 1.3632 | 0.4944 | 0.1092 |
| ATP5MPL | 0.1829 | 0.0000 | −0.0589 | −0.5950 | 0.8480 | 0.7051 | 0.6533 | −0.1349 |
| PTBP3 | −0.0339 | −0.0969 | 0.1255 | 0.3441 | 0.5639 | 0.4199 | 0.3677 | −0.2224 |
| FOSL2 | −0.0906 | −0.0199 | 0.1069 | 1.0899 | −0.2630 | 0.7981 | 0.4557 | −0.2583 |
| RPL7L1 | −0.0289 | −0.0199 | 0.1587 | 0.0070 | 0.1255 | 0.6500 | 0.1769 | 0.1561 |
| RRAD | −0.1353 | −0.0297 | −0.2224 | 1.9507 | −0.1806 | −0.0625 | −0.3410 | 0.5146 |
| VSIG2 | −0.0891 | 0.0000 | 0.1829 | 0.5305 | −0.9069 | 1.4504 | 0.4975 | 0.1740 |
| PPP1R10 | 0.1769 | 0.3505 | −0.0995 | 0.2895 | 1.0242 | 0.0971 | 0.0000 | −0.3599 |
| DOCK5 | 0.0000 | −0.1155 | −0.0780 | 0.0280 | 0.8651 | 0.4199 | 0.5261 | −0.2955 |
| REEP5 | −0.0906 | −0.0395 | 0.1587 | 0.0649 | 0.1850 | 0.4766 | 0.5564 | 0.8395 |
| TAF7 | −0.0525 | −0.2065 | 0.2955 | 0.0451 | −0.6009 | 0.7762 | 0.3973 | 0.4975 |
| CIART | 0.0651 | 0.0000 | 0.0000 | 1.2467 | −0.1623 | 0.0718 | 0.2630 | 0.1375 |
| B2M | 0.1926 | 0.0080 | −0.2090 | −1.7238 | −0.5570 | 1.0492 | 0.4444 | 0.0343 |
| SPTSSA | 0.1747 | −0.0589 | 0.4695 | 0.4374 | −0.3219 | 0.7386 | −0.2035 | −0.0995 |
| DRC3 | 0.2447 | −0.0092 | −0.2224 | 0.1644 | −0.1312 | 0.0718 | 0.3785 | 0.4150 |
| VCP | 0.1942 | −0.1740 | 0.3440 | −1.1800 | −0.1255 | 0.1788 | 0.9241 | 0.4036 |
| MTUS1 | 0.1069 | 0.1926 | 0.4475 | 0.7618 | −0.1265 | 0.2028 | −0.1312 | 0.3833 |
| SNRNP200 | 0.0759 | −0.1312 | 0.6181 | 0.2895 | 0.4227 | 0.3550 | 0.6374 | −0.1240 |
| SERINC2 | 0.1038 | −0.0395 | −0.0780 | −0.1325 | 0.2630 | 0.3151 | 0.3973 | 0.7847 |
| RSAD2 | −0.0371 | 0.3020 | −0.0780 | −0.0220 | −0.2829 | 0.3525 | 0.7678 | 0.6781 |
| MAGI3 | −0.0069 | 0.0656 | 0.4330 | −0.0544 | 0.1026 | 0.1975 | 0.3951 | 0.4735 |
| LMNA | −0.2410 | 0.1069 | −0.4739 | 0.1472 | 0.4005 | 0.4853 | 0.5741 | −0.1699 |
| ISG15 | −0.0349 | −0.0184 | −0.0780 | −0.0709 | −0.9511 | 1.0529 | 0.8480 | −0.2345 |
| DNAJC21 | 0.0489 | −0.1339 | 0.4695 | −0.3175 | 0.1864 | 0.9744 | 0.1635 | −0.0171 |
| EFHD2 | 0.0191 | 0.1155 | 0.0348 | −0.6759 | 0.0365 | 1.1663 | 1.1561 | −0.1793 |
| OASL | −0.0589 | 0.0000 | −0.0395 | 1.5305 | 0.7678 | 0.4014 | 0.2630 | 0.0902 |
| AES | 0.0924 | −0.0339 | −0.0339 | −0.6585 | 0.2124 | 1.1678 | 1.3410 | 0.3272 |
| ARPC5 | 0.0780 | −0.0969 | 0.1926 | 0.2504 | 0.0931 | 1.2704 | −0.2361 | −0.0995 |
| PTP4A2 | −0.0265 | 0.0566 | 0.0000 | −0.2471 | −0.1043 | 0.5984 | 0.3286 | 0.2487 |
| CCDC39 | −0.0958 | −0.3186 | 0.4695 | 0.0666 | 0.0000 | −0.0361 | 0.0000 | 0.3785 |
| ERCC3 | −0.0589 | 0.2065 | −0.1155 | −0.1857 | 0.0242 | 0.3025 | 0.2157 | 0.2824 |
| MT-ND3 | −0.3653 | 0.0550 | −0.9408 | 0.0304 | −0.6381 | 1.0247 | 0.6374 | 0.5329 |
| SCEL | 0.1752 | 0.0000 | 0.2224 | 0.0000 | 0.1343 | 0.3620 | 0.5439 | 0.5216 |
| CAMK1D | −0.1015 | 0.0566 | −0.0395 | −0.9720 | −0.1575 | 0.5628 | 0.3870 | −0.0704 |
| CLDN7 | 0.2503 | −0.1069 | 0.0242 | −1.0000 | 0.4210 | 0.5288 | 0.4819 | −0.0177 |
| PITPNM1 | 0.1464 | −0.2563 | 0.5208 | 0.3670 | 0.1110 | 0.3366 | −0.0217 | 0.6431 |
| ZC3H15 | −0.0875 | 0.0704 | 0.2630 | −0.3440 | 0.0825 | 0.6400 | 0.2974 | 0.1255 |
| CCND1 | 0.0684 | 0.0566 | 0.3370 | 0.0280 | 0.8371 | −0.2397 | 0.1092 | 0.0825 |
| CDCP1 | −0.0641 | 0.3370 | 0.2224 | −0.1575 | −0.5119 | −0.0699 | 0.4753 | 0.4594 |
| FAM3B | 0.0220 | 0.0759 | 0.1069 | −0.5284 | 0.4975 | 0.3374 | −0.2016 | 0.3262 |
| ELK3 | −0.0641 | 0.0375 | −0.0395 | −0.1575 | 0.0825 | 0.1439 | 0.0444 | 0.5189 |
| SLPI | 0.3053 | −0.1444 | 0.6616 | 2.0589 | −0.3611 | 0.5142 | 1.2810 | 0.6398 |
| PRR14L | 0.0208 | 0.1069 | 0.0931 | 0.0070 | 0.6063 | 0.1245 | 0.5261 | −0.3132 |
| DSP | −0.0709 | −0.0911 | −0.0589 | 0.3670 | −0.1093 | 0.8664 | 1.0784 | −0.3299 |
| CLIP1 | 0.0356 | −0.1155 | 0.2630 | 0.0395 | −0.6951 | 0.5474 | 0.4874 | −0.1322 |
| ITPR3 | −0.1155 | −0.0969 | −0.0339 | 0.7111 | 0.0375 | 0.6264 | 0.8625 | 0.1255 |
| DDR1 | 0.0759 | 0.0995 | 0.4475 | −0.5422 | −0.0740 | 0.1785 | 0.5850 | 0.1444 |
| XRCC5 | −0.0179 | −0.0995 | 0.4594 | −0.7776 | −0.3692 | 0.8443 | 0.5146 | 0.2630 |
| B3GALT5 | −0.0571 | 0.0000 | −0.0395 | 0.0000 | −0.0704 | 0.4597 | 0.7171 | 0.5670 |
| GSR | 0.0134 | 0.3561 | 0.0000 | −0.2889 | −0.0780 | 0.9206 | 0.7462 | −0.1651 |
| CTGF | 0.0173 | −0.0834 | −0.0395 | 0.4412 | 0.1699 | 0.2229 | 0.0671 | −0.0931 |
| PGD | 0.0803 | 0.0566 | 0.0931 | −0.0689 | −0.9773 | 0.9082 | 0.3370 | 0.0097 |
| PCDH1 | 0.0462 | 0.3182 | −0.0395 | −0.5475 | 0.0375 | 0.0492 | 0.6735 | 0.5585 |
| A4GALT | −0.0071 | 0.0000 | −0.1876 | 0.8301 | 0.3070 | 0.8205 | 1.1110 | 0.0704 |
| MAP3K8 | −0.0711 | 0.0759 | −0.0780 | −0.0544 | −0.2895 | 0.9215 | 0.6063 | 0.3626 |
| EGR1 | −0.1866 | −0.0395 | −0.0995 | 3.3031 | −0.4406 | 0.0614 | −0.2692 | −0.0589 |
| ATP12A | −0.0071 | 0.0000 | −0.0395 | −0.1393 | −0.1964 | 0.5548 | −0.0473 | −0.1375 |
| TXN | −0.0146 | 0.1520 | 0.1255 | −0.5229 | −0.8391 | 1.1330 | 0.0797 | 0.2487 |
| WNK1 | 0.1657 | −0.0141 | −0.0671 | −0.6539 | 0.3326 | 0.7954 | 0.4150 | 0.1699 |
| C9orf24 | 0.0919 | 0.2471 | 0.2313 | −1.6533 | 0.3219 | 0.1697 | 0.1699 | 0.5406 |
| IQGAP2 | −0.0875 | 0.1926 | 0.3626 | 0.5950 | −0.4288 | 0.2214 | 0.2025 | 0.2392 |
| LIMA1 | 0.2364 | −0.0506 | 0.4475 | −0.1393 | −0.5467 | 1.0078 | 0.9735 | −0.0506 |
| PTGES2 | 0.0000 | −0.0395 | 0.1069 | 0.0451 | 0.0995 | 0.5845 | 0.6063 | 0.5552 |
| MPRIP | 0.0067 | −0.0339 | 0.0704 | 0.0711 | −0.2035 | 0.2461 | 0.6234 | 0.1520 |
| CCDC80 | −0.0536 | −0.2546 | 0.1069 | −1.0453 | −0.2065 | 1.7328 | 0.2768 | 0.6951 |
| HMGCS1 | 0.1183 | 0.0186 | 0.0000 | −0.2768 | 0.2003 | −0.0301 | −0.3819 | 1.0885 |
| TSPAN1 | 0.0944 | 0.1120 | 0.1349 | −2.1675 | −0.6508 | 0.4970 | 0.6748 | 0.0661 |
| SPEN | −0.0891 | 0.0780 | 0.0000 | −0.0055 | 0.5850 | −0.1557 | 0.3455 | −0.3219 |
| MRPL3 | −0.0217 | 0.0956 | 0.5070 | 0.0831 | 0.7485 | 0.2594 | 0.2630 | −0.0238 |
| IL1RN | −0.1054 | 0.0000 | 0.0000 | 0.0000 | 0.3626 | 0.0485 | 0.7776 | 1.0544 |
| PRRC2C | 0.4177 | 0.1876 | 0.3262 | −0.0892 | −0.1414 | 0.5102 | 0.3996 | 0.2339 |
| EWSR1 | 0.2025 | 0.0458 | 0.2065 | 0.4374 | 0.0000 | 0.1391 | 0.3049 | 0.2487 |
| PRPF8 | 0.0191 | 0.0161 | −0.0995 | 0.7149 | 0.0704 | 0.7651 | 0.2451 | 0.6735 |
| MUC4 | −0.0473 | 0.0000 | −0.0896 | 0.4766 | −0.1189 | 0.7754 | 1.1624 | −0.4245 |
| SRRM2 | 0.1608 | −0.0780 | 0.7866 | 0.1854 | 0.0854 | 0.7695 | 0.1864 | 0.2901 |
| EPS8L1 | 0.5208 | 0.1806 | 0.4330 | 0.3190 | 0.1897 | 0.9725 | 0.9742 | −0.1251 |
| SPINK5 | 0.6850 | −0.0199 | 0.0000 | 0.0000 | 0.1343 | 0.0818 | 0.0220 | 0.9592 |
| FRMD4B | 0.0825 | 0.0525 | −0.1520 | 0.9899 | 0.5406 | 0.2453 | −0.2730 | −0.2065 |
| SERPINB2 | 0.0825 | 0.0000 | 0.1829 | 0.3606 | 0.6707 | 0.1074 | 0.8408 | −0.1454 |
| UQCR11 | 0.0515 | 0.0375 | 0.1520 | −1.4081 | 0.1333 | 0.6033 | 0.1778 | 0.2762 |
| CREBBP | −0.0825 | 0.1926 | 0.0704 | 0.4634 | 0.5291 | 0.6264 | 0.3755 | −0.2515 |
| TUBB | 0.1110 | −0.0395 | 0.1069 | 0.2310 | −0.1278 | 0.4165 | 0.6031 | −0.0969 |
| TRAPPC9 | 0.2987 | 0.2471 | −0.1520 | 0.5305 | −0.3081 | 0.1923 | 0.1699 | 0.1699 |
| NAV2 | −0.0217 | 0.0931 | 0.1926 | 0.3081 | −0.2730 | 0.0818 | 0.5639 | −0.0704 |
| CTSD | 0.1312 | −0.1520 | 0.3165 | −1.3764 | −0.6708 | 0.8951 | 0.5850 | 0.8541 |
| TPT1 | 0.4091 | −0.4150 | 0.4150 | −1.0355 | −0.0421 | 1.1435 | 0.2442 | −0.0431 |
| ATP6V0B | −0.0573 | −0.0969 | 0.5850 | −0.5475 | −0.2065 | 0.7347 | 0.6991 | 0.0679 |
| TTC9 | 0.0196 | 0.1829 | −0.0395 | −0.4695 | 0.6041 | 0.5589 | −0.1155 | 0.2295 |
| CPSF1 | −0.0431 | 0.2065 | 0.2996 | 0.1895 | 0.3720 | 0.1029 | 0.1926 | −0.0395 |
| CES2 | 0.0196 | −0.0184 | 0.0704 | 0.5305 | 0.2370 | 0.3171 | 0.1561 | 0.3262 |
| KRT6A | 0.0427 | 0.0956 | 0.0000 | 0.1520 | −0.1623 | 0.0895 | 0.2016 | 0.7227 |
| SLC2A1 | 0.1088 | −0.0395 | 0.0704 | 0.6588 | 0.0000 | 0.1301 | 0.5291 | 0.0348 |
| GAK | 0.2299 | 0.0324 | 0.1069 | −0.1575 | −0.1312 | 0.2594 | 0.2630 | −0.5850 |
| TRIM16 | −0.0661 | 0.0956 | −0.0780 | 0.5305 | 1.1085 | 0.0223 | 0.0525 | 0.5850 |
| SGK1 | −0.0623 | −0.0199 | 0.2274 | 0.6085 | 0.0566 | 0.8512 | −0.3536 | −0.2410 |
| NCF2 | 0.0725 | 0.0000 | 0.1069 | 0.3379 | 0.2345 | 0.7000 | 0.2630 | 0.0000 |
| CPEB4 | 0.0677 | 0.0772 | 0.1926 | 1.2960 | 0.4066 | 0.1381 | 0.0173 | −0.6421 |
| MAP3K13 | −0.0605 | 0.3410 | 0.1069 | 0.4150 | 0.3870 | 0.3083 | 0.3561 | 0.3720 |
| COX8A | 0.2426 | −0.0589 | 0.0000 | −0.5626 | −0.0780 | 0.5668 | 0.7866 | 0.1829 |
| PPP6R2 | 0.0358 | 0.2065 | 0.3755 | 0.1435 | −0.0473 | 0.2736 | 0.2410 | 0.1881 |
| RBM33 | −0.0217 | 0.1092 | 0.0000 | 0.8354 | 0.2801 | 0.3461 | 0.1769 | 0.2274 |
| RHOA | 0.0388 | 0.0186 | 0.2922 | −0.8594 | −0.2224 | 0.7772 | 0.5603 | 0.2705 |
| PTMA | 0.0406 | −0.2224 | −1.1844 | −0.6339 | −0.0458 | 1.2973 | 0.4800 | −0.3923 |
| GOLGA3 | 0.2699 | 0.1699 | −0.2895 | 0.0280 | 0.2003 | 1.1281 | 0.1255 | −0.1575 |
| IGFBP3 | −0.1979 | −0.0199 | −0.2563 | 0.8675 | 0.4014 | 0.5467 | 0.3605 | −0.2515 |
| METTL5 | 0.1506 | 0.1255 | 0.1444 | −0.0544 | −0.1278 | 0.4939 | 0.4557 | −0.1592 |
| PRRG4 | −0.0448 | −0.1876 | 0.0348 | −0.5915 | 0.1967 | 0.8588 | 0.2838 | 0.8321 |
| TAGLN2 | −0.0717 | 0.0956 | 0.3785 | −0.6759 | 0.0348 | 0.6155 | 0.0647 | −0.2224 |
| CD200R1 | −0.0150 | 0.0000 | 0.2224 | 0.0330 | −0.1623 | 1.3589 | 0.4258 | 0.0000 |
| FAU | 0.2406 | 0.0525 | 0.1038 | −1.2596 | −0.4150 | 1.0959 | 0.8254 | −0.2224 |
| ERBB2 | 0.1125 | 0.4066 | 0.2996 | −0.2350 | −0.2801 | 0.4639 | 0.5319 | 0.1756 |
| DDX3Y | −0.0671 | −0.0473 | 0.4330 | 0.0358 | −0.0339 | 0.0212 | 0.0220 | 0.0444 |
| PIGR | −0.5824 | −0.0297 | 0.4663 | −0.5850 | −0.4005 | 1.1780 | 1.4340 | 0.5377 |
| CFD | 0.0286 | 0.0000 | 0.0000 | 0.0451 | −0.1312 | 0.9238 | 1.2065 | 0.1137 |
| NTS | 0.2330 | 0.0000 | 0.1699 | 0.1520 | −0.3714 | 0.7962 | 0.0000 | −0.0473 |
| CD99 | 0.0196 | 0.0566 | −0.1520 | −0.3699 | 0.3523 | 0.3052 | 0.5025 | 0.2630 |
| PITPNA | −0.0711 | −0.1339 | 0.3755 | 0.2224 | −0.3219 | 0.6993 | 0.3626 | −0.1155 |
| ASAH1 | 0.0000 | 0.0375 | 0.3523 | −0.3382 | −0.0286 | 0.8087 | 0.3906 | 0.8707 |
| C1orf116 | 0.0270 | −0.0199 | 0.1829 | 0.2086 | 0.1420 | 0.6073 | 1.0000 | −0.1911 |
| ATXN2 | 0.0515 | −0.1265 | 0.2313 | 0.2675 | 0.7105 | 0.4776 | −0.2429 | 0.0000 |
| NPEPPS | 0.2168 | 0.0656 | 0.4695 | −0.2630 | 0.3626 | 0.1036 | 0.3149 | 0.1143 |
| PPP6R3 | −0.0360 | 0.1881 | 0.1069 | 0.3081 | 0.4274 | −0.0561 | 0.2838 | 0.2630 |
| SFN | 0.1328 | 0.0000 | −0.0780 | 0.5305 | −0.1575 | 0.1265 | 0.4297 | 0.6181 |
| GALNT5 | −0.1054 | −0.0199 | −0.1520 | 0.1520 | 0.1520 | 0.8822 | 1.0415 | 0.3833 |
| HK1 | 0.1289 | 0.2462 | 0.2224 | −0.3764 | −0.8480 | 0.5561 | 0.6735 | 0.4044 |
| KRT8 | 0.3219 | −0.0995 | 0.1255 | −0.6801 | −0.1106 | 0.6107 | 0.3677 | 0.0000 |
| PARP14 | 0.0845 | 0.2534 | 0.0931 | 0.4739 | 0.3801 | 0.4520 | 0.3572 | 0.3219 |
| ABHD5 | 0.3191 | 0.1444 | −0.0780 | −0.6070 | 0.3081 | 0.0647 | 0.4579 | 0.3505 |
| OXTR | 0.0073 | 0.0759 | −0.0395 | −0.1069 | 0.0000 | 0.2410 | 0.1375 | 0.1375 |
| RPS12 | 0.3238 | 0.2630 | 0.1699 | −0.6659 | 0.1728 | 0.9154 | 1.4411 | −0.3177 |
| ANXA11 | 0.1141 | 0.2974 | 0.1699 | −0.5950 | 0.0647 | 0.9906 | 0.8615 | 0.1057 |
| SF3A1 | 0.2204 | −0.0395 | 0.1069 | 0.1155 | 0.1618 | 0.3418 | 0.2426 | 0.1635 |
| CCDC40 | 0.0566 | 0.3806 | 0.4695 | −0.4106 | −0.1623 | 0.2410 | 0.2392 | 0.1375 |
| SCO2 | −0.0780 | −0.4975 | −0.1155 | 0.7190 | 0.3561 | 0.2880 | 0.5850 | 0.1635 |
| SPRR2A | 0.8377 | −0.0199 | 0.0000 | 0.0000 | −0.0995 | −0.0181 | 0.3093 | 0.9084 |
| ACAT2 | 0.1945 | 0.0000 | 0.5454 | −0.4695 | 0.1402 | 0.1599 | 0.1255 | 0.0825 |
| P4HB | 0.2178 | 0.2224 | 0.2313 | −0.2350 | −0.7026 | 0.6859 | 0.7710 | 0.5146 |
| NFKBIA | 0.2088 | −0.0896 | −0.2730 | 1.8061 | −0.1876 | 0.4254 | −0.1699 | −0.2964 |
| C6orf132 | −0.1501 | 0.0173 | 0.4150 | −0.5113 | −0.1349 | 0.7542 | 0.3755 | 0.6616 |
| PSMD2 | 0.0890 | 0.0375 | 0.1587 | −0.0752 | −0.3692 | 0.5964 | 0.2182 | 0.2204 |
| CEBPD | 0.0871 | 0.0000 | 0.0704 | 0.2310 | −0.0120 | 1.2109 | 0.2274 | −0.2651 |
| RPL32 | 0.0427 | 0.0000 | −0.0265 | −0.4695 | 0.1904 | 1.1970 | 1.0176 | 0.2630 |
| ZFAND5 | −0.1240 | −0.1520 | 0.3833 | −0.1409 | 0.2738 | 0.3451 | 0.2494 | 0.8420 |
| CHD4 | 0.0938 | 0.1146 | −0.3081 | 0.0916 | −0.1155 | 0.4589 | 1.1790 | −0.2611 |
| POR | 0.1078 | 0.1967 | 0.2065 | 0.4050 | −0.6534 | 0.8205 | 0.3462 | 0.2863 |
| MT-ATP6 | 0.5490 | −0.2584 | −0.1224 | −0.5120 | −0.4027 | −0.0085 | 0.6685 | 0.6809 |
| EIF2AK2 | 0.0597 | 0.1587 | 0.0000 | −0.1158 | −0.1155 | 0.9756 | 0.9829 | 0.1255 |
| TUBB4B | 0.1749 | −0.5070 | 0.1747 | −1.3925 | −0.7472 | 0.7437 | −0.8413 | 0.5493 |
| AKR1C2 | −0.1054 | 0.0956 | 0.0304 | 0.6725 | −0.4874 | 0.9937 | 0.8745 | −0.1806 |
| RBP1 | −0.0945 | 0.3370 | 0.0000 | −0.3175 | −0.2224 | 0.9416 | 0.6077 | 0.0000 |
| SPRR2E | 0.2283 | 0.0000 | 0.0000 | −0.2065 | −0.0339 | 0.0000 | −0.0238 | 2.5124 |
| SPRR1B | 1.0000 | 0.1155 | 0.2224 | 0.0451 | 0.1255 | 0.0895 | 0.3626 | 0.2416 |
| PSMD11 | −0.0589 | 0.0186 | 0.2955 | −0.0910 | 0.4352 | 0.8133 | 0.4579 | −0.0184 |
| BAG1 | 0.0397 | −0.0589 | 0.4594 | 0.0525 | −0.1822 | 0.3784 | 0.0110 | 0.2846 |
| CYP1B1 | −0.0224 | −0.0199 | 0.0000 | 0.2086 | 0.0489 | 1.6220 | 0.9260 | 0.0000 |
| SAMD9 | 0.1764 | 0.0566 | 0.4475 | 0.2086 | −0.6280 | 1.0548 | 0.1255 | 0.1790 |
| ALDH1A3 | −0.0641 | 0.2224 | 0.1444 | 0.1679 | 0.3119 | 0.3418 | −0.0533 | 0.8007 |
| OGFR | 0.0489 | 0.1635 | 0.0000 | 0.7111 | 0.0270 | 0.2005 | 0.4919 | −0.4634 |
| NLRC5 | 0.1747 | −0.0395 | 0.5850 | 0.2310 | 0.4919 | 0.3418 | 0.7182 | 0.3149 |
| PFKP | −0.0069 | 0.1069 | −0.0395 | 0.5894 | 0.0995 | −0.0348 | 0.5110 | −0.0199 |
| PARP9 | 0.0277 | −0.3999 | −0.1155 | −0.0825 | −0.1220 | 0.6447 | 0.9635 | 0.7859 |
| CEACAM6 | 0.0656 | 0.0000 | 0.2274 | 0.0000 | −0.0902 | 1.1923 | 1.0881 | 1.0986 |
| MYO5B | 0.2370 | 0.1255 | 0.2224 | 0.8386 | −0.3650 | 0.4423 | 0.4639 | 0.3479 |
| STAT3 | 0.2439 | 0.2451 | −0.0297 | −0.1216 | −0.1640 | 0.5224 | 0.1587 | 0.5159 |
| SLC4A11 | −0.0473 | 0.0525 | −0.3536 | −0.0314 | 1.1762 | −0.0235 | 1.0000 | −0.0100 |
| KLF6 | −0.2462 | 0.1829 | −0.0995 | 0.9682 | −0.5255 | 0.9531 | −0.4854 | 0.6666 |
| CCDC57 | 0.0270 | 0.1459 | 0.0304 | 0.6460 | 0.1769 | 0.5606 | 0.0202 | 0.3699 |
| TRIM8 | −0.1285 | 0.1699 | 0.0704 | −0.6318 | −0.0171 | 0.5324 | 0.2974 | 0.5261 |
| MSMB | 0.3536 | 0.0000 | 0.5110 | 0.9456 | −0.2290 | 1.3229 | 1.3444 | 0.0772 |
| SPRR3 | 1.3077 | 0.1699 | −0.0395 | −0.1476 | 0.5611 | −0.4036 | −0.4150 | 0.2324 |
| TMPRSS4 | −0.0604 | −0.0721 | 0.9329 | 0.8842 | −0.3766 | 0.8119 | 0.7341 | −0.2315 |
| MVK | 0.0220 | −0.0199 | 0.7370 | 0.2086 | 0.4150 | 0.2121 | 0.1476 | 0.0444 |
| CTSH | 0.0477 | −0.1575 | −0.1520 | −0.3820 | 0.0577 | 0.8138 | 0.7406 | 0.3370 |
| KRT4 | 0.0304 | 0.0000 | 0.4806 | −0.1069 | 1.1653 | −1.2431 | 0.8147 | 1.3251 |
| RPL37 | 0.0304 | 0.0885 | −0.1031 | −0.9289 | 0.1155 | 1.3430 | 1.1164 | 0.0875 |
| ASRGL1 | 0.2204 | 0.1444 | 0.4297 | −0.3175 | 0.0956 | 0.7164 | 0.2801 | 0.2157 |
| CSNK1D | −0.0671 | 0.2274 | −0.1876 | 0.9741 | 0.2479 | 0.4406 | 0.5227 | 0.1901 |
| KRT7 | 0.1151 | 0.1155 | 0.0220 | 0.9899 | 0.0402 | 0.3016 | 0.8504 | 0.9723 |
| S100A14 | 0.0406 | 0.1155 | 0.1255 | −0.2996 | 0.2115 | 0.7094 | 0.4304 | 1.0156 |
| MACC1 | 0.1743 | −0.1699 | 0.5525 | −0.4695 | 0.6506 | 0.7289 | 0.6859 | −0.2865 |
| MT-CO2 | −0.0659 | −0.1443 | −0.2785 | −0.2082 | −0.3949 | 0.5069 | 0.9202 | 0.8382 |
| ARHGAP5 | −0.0704 | 0.1069 | 0.1699 | −0.1832 | 0.7726 | 0.4498 | 0.0000 | 0.3415 |
| TRIM29 | 0.1679 | −0.0395 | 0.0348 | 0.6207 | −0.1155 | 0.6220 | 0.5694 | 0.5850 |
| MUC1 | 0.4711 | −0.3692 | −0.0641 | −1.1248 | −0.1578 | 1.0748 | 1.8911 | 0.1685 |
| SLC25A3 | −0.1043 | 0.0186 | 0.1255 | 0.0759 | 0.0566 | 0.4740 | 0.5208 | 0.3340 |
| PYGL | 0.0940 | −0.0395 | −0.2895 | 0.0899 | 0.6888 | 0.4458 | 0.6831 | 0.1110 |
| MUC20-OT1 | 0.1424 | 0.0684 | −0.0671 | 0.1679 | 0.4826 | 0.6173 | 0.4630 | 0.0489 |
| ACTN4 | 0.1520 | 0.0324 | −0.2224 | 0.0634 | −0.2439 | 0.9287 | 1.2838 | 0.1349 |
| S100A10 | 0.0397 | 0.0000 | −0.0265 | −0.3739 | 0.1459 | 0.5467 | 0.0995 | 0.5984 |
| KRT24 | 0.4381 | 0.1155 | −0.3890 | 0.0000 | 2.6242 | −0.7686 | −0.8225 | 2.0995 |
| GAN | 0.1069 | 0.0566 | 0.3755 | 0.3499 | 0.1255 | 1.0172 | 0.5431 | 0.2943 |
| GLUL | 0.4216 | 0.1520 | −0.2775 | 0.5871 | 1.1305 | 1.0553 | 0.0465 | −0.0708 |
| IFI44L | −0.0217 | −0.1339 | 0.0000 | 0.1520 | −0.0633 | 1.1039 | 0.0956 | 0.1926 |
| RDH10 | 0.1287 | 0.0000 | 0.4005 | 0.5305 | −0.9125 | 1.5863 | 0.1312 | 1.2050 |
| KRT19 | 0.8251 | −0.0780 | 0.3286 | −1.0234 | 0.1612 | 0.7386 | 1.1260 | 0.1327 |
| EZR | −0.1061 | 0.2065 | −0.5983 | −0.1770 | −0.1709 | 0.8881 | 0.8845 | 0.7196 |
| IFITM1 | −0.0517 | 0.1635 | 0.0000 | 0.7462 | −0.2224 | 0.4400 | 0.0000 | 0.2854 |
| FLNB | 0.1858 | 0.0704 | 0.4330 | −0.4206 | 0.2242 | 0.2080 | 1.0473 | 0.1699 |
| S100P | 0.9832 | −0.2065 | 1.0679 | −0.6183 | −0.4135 | 1.0604 | 1.6119 | 0.2608 |
| MT-ND5 | 0.0137 | −0.4471 | 0.1614 | −0.3691 | −0.4981 | 0.2357 | 0.2656 | 0.5619 |
| AHNAK2 | 0.0647 | 0.0740 | −0.0395 | 0.2675 | 0.3219 | 0.4240 | 1.0656 | 0.0000 |
| TNFAIP3 | −0.0952 | 0.0186 | −0.0995 | 3.8524 | 0.3626 | 0.1760 | −0.0544 | −0.1876 |
| TBC1D8 | −0.1305 | −0.0238 | −0.0780 | 1.1249 | 0.0956 | 0.7981 | 0.2193 | −0.0431 |
| S100A9 | 1.0902 | 0.5510 | 0.2922 | −0.2996 | −0.1096 | −0.7912 | −0.0255 | −0.1977 |
| OS9 | 0.0845 | −0.0184 | 0.4695 | −0.0289 | 0.3165 | 0.8443 | 0.7687 | 0.7705 |
| S100A6 | 0.5081 | −0.4418 | 0.2285 | −0.9813 | −1.0129 | 1.8943 | 0.2241 | −0.4003 |
| CEACAM5 | −0.5394 | 0.0956 | 0.1255 | 0.0000 | −0.8131 | 3.0249 | 0.7898 | 1.6226 |
| SPINT2 | 0.0000 | 0.2451 | 0.7370 | −0.7105 | −0.2419 | 0.8443 | 1.0641 | −0.3569 |
| ACTG1 | 0.1969 | −0.0506 | −0.3479 | −0.6007 | −0.0943 | 1.0255 | 0.7586 | −0.0894 |
| MT-CYB | 0.5187 | −0.1075 | 0.1444 | −0.7794 | −0.4036 | −0.0684 | 0.3399 | 0.6903 |
| MT-ND1 | 0.4979 | −0.4377 | −0.1532 | 0.0500 | −0.2897 | −0.0449 | 0.1921 | 0.4972 |
| MUC21 | 0.2908 | 0.0000 | 0.1829 | 0.0000 | 0.8546 | 0.5065 | 0.5406 | 1.9309 |
| MT-CO3 | 0.1468 | −0.3293 | −0.2119 | 0.2904 | −0.4206 | 0.3062 | 0.7742 | 0.7099 |
| LRRFIP1 | 0.2209 | 0.2193 | −0.3626 | −0.2966 | 0.5633 | 0.9932 | 0.9538 | 0.1795 |
| PPL | −0.0234 | −0.0671 | −0.1312 | 0.6858 | 0.4826 | 0.4458 | 1.4411 | 0.2529 |
| ELF3 | 0.2093 | 0.0115 | −0.4150 | 0.6673 | −0.0249 | 0.1452 | 0.1587 | 0.1997 |
| MX1 | 0.3194 | 0.1402 | −0.0589 | −0.1513 | −0.9086 | 1.3488 | 0.6542 | −0.1409 |
| MTRNR2L1 | −0.0381 | 0.1333 | −0.1031 | −0.5201 | 0.2141 | 0.6288 | 0.7687 | 0.0000 |
| F3 | 0.1162 | −0.1069 | 0.6739 | 0.5305 | 0.0863 | 0.9841 | −0.3370 | 1.5281 |
| MTRNR2L12 | 0.5095 | −0.1185 | 0.1318 | −0.2554 | 0.1598 | −0.0231 | 0.6717 | −0.0748 |
| WFDC2 | −0.0843 | 0.0577 | 0.7105 | −1.5553 | −0.5140 | 1.2132 | 1.5958 | 0.2370 |
| MT-ND6 | 0.2630 | −0.0473 | 0.5431 | 0.2675 | −0.5555 | 0.1360 | 0.6636 | 0.2630 |
| PER1 | 0.1506 | 0.0000 | −0.0395 | 2.2051 | 0.4420 | 0.6821 | 0.2193 | −0.2955 |
| PLEC | 0.1654 | −0.2659 | 0.2996 | 0.5712 | 0.7335 | 0.5321 | 1.1444 | 0.5850 |
| TACSTD2 | 0.3779 | −0.1876 | 0.6256 | −1.0074 | 0.1122 | 0.4109 | 1.4565 | 0.3233 |
| LMO7 | 0.2354 | 0.2630 | −0.0265 | 0.2164 | 0.1255 | 1.1439 | 1.5253 | 0.5827 |
| AHNAK | 0.1646 | 0.0073 | 0.7105 | −0.1699 | 0.2389 | 0.6111 | 0.8771 | 0.4975 |
| IFI27 | 0.3272 | 0.4285 | −0.0184 | −0.8398 | −0.6795 | 1.1824 | 1.5645 | −0.4072 |
| IFITM3 | 0.2563 | 0.0704 | 0.2630 | −0.3508 | −0.0378 | 1.0907 | 1.1954 | 0.1420 |
| LGALS3 | 0.1409 | 0.3523 | 0.5850 | −0.8222 | 0.0342 | 1.6517 | 1.0298 | 0.1289 |
| PSCA | 0.3116 | 0.2035 | 0.5168 | −1.2269 | −0.4596 | 1.2544 | 1.6953 | 0.0761 |
| IFI6 | 0.6764 | 0.1756 | −0.1312 | −0.6366 | −1.0875 | 2.2083 | 0.3655 | −0.0614 |
| MUC5AC | 0.0057 | 0.0186 | 2.4837 | 0.8106 | 0.5744 | 1.7880 | 2.1786 | 0.0000 |

TABLE 6
Participant characteristics

| | Control | Intubated Control | COVID-19 m/m | COVID-19 severe | COVID-19 conv. |
|---|---|---|---|---|---|
| | (WHO score 0) | (WHO score 7-8) | (WHO score 1-5) | (WHO score 6-8) | (WHO score 0) |
| Case number | 25.9% (15/58) | 10.3% (6/58) | 24.1% (14/58) | 36.2 (21/58) | 3.4% (2/58) |
| Age (years) | | | | | |
| Minimum | 27 | 33 | 19 | 28 | 20 |
| Median (IQR) | 58 (16) | 65.5 (31) | 49.5 (17.8) | 62 (13) | N/A |
| Maximum | 73 | 71 | 69 | 84 | 57 |
| Sex | | | | | |
| Female | 60% (9/15) | 16.7% (1/6) | 42.9% (6/14) | 47.6% (10/21) | 50% (1/2) |
| Male | 40% (6/15) | 83.3% (5/6) | 57.1% (8/14) | 52.4% (11/21) | 50% (1/2) |
| Ethnicity | | | | | |
| Hispanic | 0% (0/15) | 0% (0/6) | 0% (0/14) | 4.8% (1/21) | 0% (0/2) |
| Not Hispanic | 100% (15/15) | 100% (6/6) | 100% (14/14) | 95.2% (20/21) | 100% (2/2) |
| Race | | | | | |
| Black/African American | 66.7% (10/15) | 66.7% (4/6) | 71.4% (10/14) | 61.9% (13/21) | 50% (1/2) |
| White | 33.3% (5/15) | 33.3% (2/6) | 28.6% (4/14) | 23.8% (5/21) | 50% (1/2) |
| American Indian | 0% (0/15) | 0% (0/6) | 0% (0/14) | 14.3% (3/21) | 0% (0/2) |
| BMI | | | | | |
| Median (IQR) | 37.5 (14.4) | 30.5 (18.1) | 23.0 (11.6) | 31.9 (14.2) | 40.7 |
| Pre-existing conditions | | | | | |
| Diabetes | 40% (6/15) | 33.3% (2/6) | 28.6% (4/14) | 71.4% (15/21) | 0% (0/2) |
| Chronic kidney disease | 6.7% (1/15) | 0% (0/6) | 7.1% (1/14) | 19.0% (4/21) | 0% (0/2) |
| Congestive heart failure | 6.7% (1/15) | 16.7% (1/6) | 0% (0/14) | 4.8% (1/21) | 0% (0/2) |
| Lung disorder | 6.7% (1/15) | 16.7% (1/6) | 28.6% (4/14) | 38.1% (8/21) | 0% (0/2) |
| Hypertension | 86.7% (13/15) | 50% (3/6) | 42.9% (6/14) | 81.0% (17/21) | 0% (0/2) |
| IBD | 13.3% (2/15) | 0% (0/6) | 0% (0/14) | 0% (0/21) | 50% (1/2) |
| Treatment | | | | | |
| Corticosteroids | N/A | 33.3% (2/6) | 42.9% (6/14) | 66.7% (14/21) | N/A |
| Remdesivir | N/A | 0% (0/6) | 42.9% (6/14) | 85.7% (18/21) | N/A |
| 28-day mortality | 0% (0/15) | 33.3% (2/6) | 0% (0/14) | 76.2% (16/21) | 0% (0/2) |

m/m: mild/moderate
conv: convalescent
IQR: inter-quartile range
BMI: body mass index
IBD: inflammatory bowel disease

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed:

1. A method of treating a barrier tissue infection in a subject in need thereof comprising:
detecting one or more indicators of infection from a sample obtained from the subject, wherein the sample comprises one or more of epithelial, immune, stromal, and neuronal cells;
comparing the indicators to control/healthy samples or disease reference values to determine whether the subject will progress to a risk group selected from:
(i) mild/moderate disease; or
(ii) severe disease; and
administering one or more treatments if one or more indicators are present.

2. The method of claim 1, wherein the barrier tissue infection is a respiratory barrier tissue infection.

3. The method of claim 2, wherein mild subjects are asymptomatic or symptomatic and not hospitalized, wherein moderate subjects are hospitalized and do not require oxygen by non-invasive ventilation or high flow, and wherein severe subjects are hospitalized and require oxygen by non-invasive ventilation, high flow, or intubation and mechanical ventilation.

4. The method of any of claims 1 to 3, wherein the infection is a viral infection.

5. The method of claim 4, wherein the viral infection is a coronavirus.

6. The method of claim 5, wherein the coronavirus is SARS-CoV2 or variant thereof.

7. The method of claim 6, wherein mild/moderate subjects have a WHO score of 1-5 and severe subjects have a WHO score of 6-8.

8. The method of any of claims 1 to 7, wherein the one or more indicators of infection are selected from the group consisting of:
a) decreased interferon-stimulated gene (ISG) induction;
b) upregulation of one or more anti-viral factors or IFN-responsive genes;
c) reduction of mature ciliated cell population or increased immature ciliated cell population;
d) increased secretory cell population;
e) increased deuterosomal cell population;
f) increased ciliated cell population;
g) increased goblet cell population;
h) decreased expression in Type II interferon specific genes;
i) increased expression in Type I interferon specific genes;
j) increased MHC-I and MHC-II genes;
k) increased developing ciliated cell populations;
l) altered expression of one or more genes in a cell type selected from any of Tables 2-4;
m) altered expression of one or more genes in a cell type selected from Table 5;
n) increase expression of IFITM3 and IFI44L;
o) increased expression of EIF2AK2;
p) increased expression of TMPRSS4, TMPRSS2, CTSS, CTSD;
q) upregulation of cholesterol and lipid biosynthesis; and
r) increased abundance of low-density lipoprotein receptors LDLR and LRP8.

9. The method of claim 8, wherein one or more interferon-stimulated genes are detected, wherein if the one or more interferon-stimulated genes are downregulated the subject is at risk for severe disease and if the one or more interferon-stimulated genes are upregulated the subject is not at risk for severe disease.

10. The method of claim 9, wherein the one or more interferon-stimulated genes are selected from the group consisting of STAT1, STAT2, IRF1, and IRF9.

11. The method of any of claims 1 to 10, wherein the one or more indicators of infection are detected in infected host cells and compared to reference values in infected host cells from a risk group.

12. The method of claim 11, wherein one or more anti-viral factors or IFN-responsive genes are detected in virally-infected cells, wherein if the one or more anti-viral factors or IFN-responsive genes are downregulated or absent in virally-infected cells the subject is at risk for severe disease and if the one or more anti-viral factors or IFN-responsive genes are upregulated in virally-infected cells the subject is not at risk for severe disease.

13. The method of claim 12, wherein the one or more anti-viral factors or IFN-responsive genes are selected from the group consisting of EIF2AK2, STAT1 and STAT2.

14. The method of any of claims 8 to 13, wherein the secretory cells comprise one or both of: KRT13 KRT24 high Secretory Cells and Early Response Secretory Cells.

15. The method of any of claims 8 to 13, wherein the secretory cells express CXCL8.

16. The method of any of claims 8 to 13, wherein the goblet cells comprise one or both of: AZGP1 high Goblet Cells and SCGB1A1 high Goblet Cells.

17. The method of any of claims 8 to 13, where the ciliated cells comprise one or more upregulated genes selected from the group consisting of IFI27, IFIT1, IFI6, IFITM3, and GBP3.

18. The method of any of claims 8 to 13, wherein one or both of the ciliated cells and the goblet cells comprise increased gene expression of one or more IFN gene selected from any of Tables 2-4.

19. The method of any of claims 8 to 13, wherein ACE2 expression is upregulated compared to other epithelial cells among one or more of secretory cells, goblet cells, ciliated cells, developing ciliated cells, and deuterosomal cells.

20. The method of any of claims 8 to 13, wherein the mature ciliated cells are BEST4 high cilia high ciliated cells.

21. The method of any of claims 8 to 13, wherein the MHC-I and MHC-II genes comprise at least one or more of: HLA-A, HLA-C, HLA-F, HLA-E, HLA-DRB1, and HLA-DRA.

22. The method of any of claims 8 to 13, wherein the upregulated cholesterol and lipid biosynthesis genes comprise at least one or more of: FDFT1, MVK, FDPS, ACAT2, and HMGCS1.

23. The method of any of claims 1 to 22, wherein detecting one or more indicators is performed by using Simpson's index.

24. The method of any of claims 1 to 23, where a subject will progress to the severe risk group if one or more of the following is detected in the sample:
a) proinflammatory cytokines comprising at least one or more of: IL1B, TNF, CXCL8, CCL2, CCL3, CXCL9, CXCL10, and CXCL11;
b) upregulation of alarmins comprising one or both of: S100A8 and S100A9;
c) 14%-26% of all epithelial cells are secretory cells;
d) elevated BPIFA1 high Secretory cells;
e) elevated KRT13 KRT24 high secretory cells;
f) macrophage population increase as compared to other immune cells;
g) upregulated genes in ciliated cells comprising one or both of: IL5RA and NLRP1;
h) no increase of at least one or more of: type I, type II, and type III interferon abundance;
i) elevated stress response factors comprising at least one or more of: HSPA8, HSPA1A, and DUSP1;
j) increased expression of one or more genes differentially expressed in COVID-19 WHO 6-8 according to Table 3 or Table 4;
k) reduced or absent antiviral/interferon response; and
l) reduced or absent mature ciliated cells.

25. The method of claim 24, wherein the macrophage population comprises at least one or more of: ITGAX High Macrophages, FFAR High Macrophages, Inflammatory Macrophages, and Interferon Responsive Macrophages.

26. The method of any of claims 1 to 23, where a subject is determined to belong to the mild/moderate risk group if one or more of the following is detected in the sample:
a) 4%-12% of all epithelial cells are Secretory Cells;
b) 10%-20% of all epithelial cells comprise Interferon Responsive Ciliated Cells;
c) upregulated ciliated cell genes comprising at least one or more of: IFI44L, STAT1, IFITM1, MX1, IFITM3, OAS1, OAS2, OAS3, STAT2, TAP1, HLA-C, ADAR, XAF1, IRF1, CTSS, and CTSB;
d) increase in type I interferon abundance;
e) high expression of interferon-responsive genes;
f) decreased expression of one or more genes differentially expressed in COVID-19 WHO 6-8 according to Table 3 or Table 4;
g) induction of type I interferon responses; and
h) high abundance of IFI6 and IFI27.

27. The method of claim 26, where the interferon-responsive genes comprise at least one or more of: STAT1, MX1, HLA-B, and HLA-C.

28. The method of claim 26, where the interferon response occurs in at least one or more of: MUC5AC high Goblet Cells, SCGB1A1 high Goblet Cells, Early Response Secretory Cells, Deuterosomal Cells, Interferon Responsive Ciliated Cells, and BEST4 high Cilia high Ciliated Cells.

29. The method of any of claims 1 to 28, wherein the treatment is administered according to determined risk group.

30. The method of claim 29, where the treatment involves administering a preventative or therapeutic intervention according to the determined risk group.

31. The method of claim 29 or 30, wherein if the subject is determined to be at risk for progression to the severe risk group the subject is administered a treatment comprising one or more treatments selected from the group consisting of:
a) one or more antiviral;
b) blood-derived immune-based therapy;
c) one or more corticosteroid;
d) one or more interferon;
e) one or more interferon Type I agonists;
f) one or more interleukin-1 inhibitors;
g) one or more kinase inhibitors;
h) one or TLR agonists;
i) a glucocorticoid; and
j) interleukin-6 inhibitor.

32. The method of claim 29 or 30, wherein if the subject is determined to be at risk for progression to either risk group the subject is administered a treatment comprising one or more of:
a) one or more antiviral;
b) one or more antibiotic; and
c) one or more cholesterol biosynthesis inhibitor.

33. The method of claim 29 to 32, where the treatment comprises an antiviral.

34. The method of the 33, where the antiviral inhibits viral replication.

35. The method of claim 34, where the antiviral is selected from the group consisting of paxlovid, molnupiravir and remdesivir.

36. The method of claim 29 to 32, where the treatment is an immune-based therapy.

37. The method of claim 36, where the immune-based therapy is a blood-derived product comprising at least one or more of: a convalescent plasma and an immunoglobin.

38. The method of claim 37, where the immune-based therapy is an immunomodulator comprising at least one or more of: a corticosteroid, a glucocorticoid, an interferon, an interferon Type I agonist, an interleukin-1 inhibitor, an interleukin-6 inhibitor, a kinase inhibitor, and a TLR agonist.

39. The method of the claim 38, where the corticosteroid comprises at least one of: methylprednisolone, hydrocortisone, and dexamethasone.

40. The method of the claim 38, where the glucocorticoid comprises at least one of: cortisone, prednisone, prednisolone, methylprednisolone, dexamethasone, betamethasone, triamcinolone, Fludrocortisone acetate, deoxycorticosterone acetate, and hydrocortisone.

41. The method of claim 38, where the interferon comprises at least one or more of: interferon beta-1b and interferon alpha-2b.

42. The method of claim 38, where the interleukin-1 inhibitor comprises anakinra.

43. The method of claim 38, where the interleukin-6 inhibitor comprises at least one or more of: anti-interleukin-6 receptor monoclonal antibodies and anti-interleukin-6 monoclonal antibody.

44. The method of the claim 43, where the anti-interleukin-6 receptor monoclonal antibody is tocilizumab.

45. The method of the claim 43, where the anti-interleukin-6 monoclonal antibody is siltuximab.

46. The method of the claim 38, where the kinase inhibitor comprises of at least one or more of Bruton's tyrosine kinase inhibitor and Janus kinase inhibitor.

47. The method of claim 46, where the Bruton's tyrosine kinase inhibitor comprises at least one or more of: acalabrutinib, ibrutinib, and zanubrutinib.

48. The method of claim 46, where the Janus kinase inhibitor comprises at least one or more of: baracitinib, ruxolitinib and tofacitinib.

49. The method of claim 38, were the TLR agonist comprises at least one or more of: imiquimod, BCG, and MPL.

50. The method of claim 29 to 32, wherein the treatment comprises inhibiting cholesterol biosynthesis.

51. The method of claim 50, wherein inhibiting cholesterol biosynthesis comprises administering HMG-CoA reductase inhibitors.

52. The method of 51, wherein the HMG-CoA reductase inhibitor comprises at least one or more of: simvastatin atorvastatin, lovastatin, pravastatin, fluvastatin, rosuvastatin, pitavastatin.

53. The method of any claim 29 to 32, where the treatment comprises an antibiotic.

54. The method of claim 1, wherein the treatment comprises one or more agents capable of shifting epithelial cells to express an antiviral signature.

55. The method of claim 1, wherein the treatment comprises one or more agents capable of suppressing a myeloid inflammatory response.

56. The method of claim 1, wherein the treatment comprises a CRISPR-Cas system.

57. The method of claim 56, wherein the CRISPR system comprises a CRISPR-Cas base editing system, a prime editor system, or a CAST system.

58. The method of any of the preceding claims, wherein the treatment is administered before disease onset.

59. The method of any of the preceding claims, wherein the one or more cell types are detected using one or markers differentially expressed in the cell types.

60. The method of any of the preceding claims, wherein the one or more cell types or one or more genes are detected by immunohistochemistry (IHC), fluorescence activated cell sorting (FACS), fluorescently bar-coded oligonucleotide probes, RNA FISH (fluorescent in situ hybridization), RNA-seq, or any combination thereof.

61. The method of claim 60, wherein single cell expression is inferred from bulk RNA-seq.

62. The method of claim 61, wherein expression is determined by single cell RNA-seq.

63. A method of screening for agents capable of shifting epithelial cells from a SARS-CoV2 severe phenotype to a mild/moderate phenotype comprising:
a. treating a sample comprising epithelial cells with a drug candidate;
b. detecting modulation of any indicators of infection according to any of the preceding claims; and
c. identifying the drug, wherein the one or more indicators shift towards a mild/moderate phenotype.

64. The method of claim 63, wherein the sample comprises epithelial cells infected with SARS-CoV2.

65. The method of claim 63, wherein the sample comprises epithelial cells expressing one or more SARS-CoV2 genes.

66. The method of any of claims 63 to 65, wherein the sample is an organoid or tissue model.

67. The method of any of claims 63 to 65, wherein the sample is an animal model.

68. The method of any of the preceding claims, wherein cell types are detected using one or markers selected from Table 1.

69. A method of detecting susceptibility to a barrier tissue infection in a subject in need thereof comprising:
detecting one or more indicators of susceptibility from a sample obtained from the subject, wherein the sample comprises one or more of epithelial, immune, stromal, and neuronal cells;
comparing the indicators to control/healthy samples or disease reference values to determine whether the subject belongs to a risk group selected from mild/moderate; or severe.

70. The method of claim 69, wherein the barrier tissue infection is a respiratory barrier tissue infection.

71. The method of claim 70, wherein mild subjects are asymptomatic or symptomatic and not hospitalized, wherein moderate subjects are hospitalized and do not require oxygen by non-invasive ventilation or high flow, and wherein severe subjects are hospitalized and require oxygen by non-invasive ventilation, high flow, or intubation and mechanical ventilation.

72. The method of any of claims 69 to 71, wherein the infection is a viral infection.

73. The method of claim 72, wherein the viral infection is a coronavirus.

74. The method of claim 73, wherein the coronavirus is SARS-CoV2 or variant thereof.

75. The method of claim 74, wherein mild/moderate subjects have a WHO score of 1-5 and severe subjects have a WHO score of 6-8.

76. The method of any of claims 69 to 75, wherein the one or more indicators of susceptibility are selected from the group consisting of:
a) decreased interferon-stimulated gene (ISG) induction;
b) upregulation of one or more anti-viral factors or IFN-responsive genes;
c) reduction of mature ciliated cell population or increased immature ciliated cell population;
d) increased secretory cell population;
e) increased deuterosomal cell population;
f) increased ciliated cell population;
g) increased goblet cell population;
h) decreased expression in Type II interferon specific genes;
i) increased expression in Type I interferon specific genes;
j) increased MHC-I and MHC-II genes;
k) increased developing ciliated cell populations;
l) altered expression of one or more genes in a cell type selected from any of Tables 2-4;
m) altered expression of one or more genes in a cell type selected from Table 5;
n) increase expression of IFITM3 and IFI44L;
o) increased expression of EIF2AK2;
p) increased expression of TMPRSS4, TMPRSS2, CTSS, CTSD;
q) upregulation of cholesterol and lipid biosynthesis; and
r) increased abundance of low-density lipoprotein receptors LDLR and LRP8.

77. The method of claim 76, wherein one or more interferon-stimulated genes are detected, wherein if the one or more interferon-stimulated genes are downregulated the subject is at risk for severe disease and if the one or more interferon-stimulated genes are upregulated the subject is not at risk for severe disease.

78. The method of claim 77, wherein the one or more interferon-stimulated genes are selected from the group consisting of STAT1, STAT2, IRF1, and IRF9.

79. The method of any of claims 69 to 78, wherein the one or more indicators of infection are detected in infected host cells and compared to reference values in infected host cells from a risk group.

80. The method of claim 79, wherein one or more anti-viral factors or IFN-responsive genes are detected in virally-infected cells, wherein if the one or more anti-viral factors or IFN-responsive genes are downregulated or absent in virally-infected cells the subject is at risk for severe disease and if the one or more anti-viral factors or IFN-responsive genes are upregulated in virally-infected cells the subject is not at risk for severe disease.

81. The method of claim 80, wherein the one or more anti-viral factors or IFN-responsive genes are selected from the group consisting of EIF2AK2, STAT1 and STAT2.

82. The method of claim 70, wherein the secretory cells comprise one or both of: KRT13 KRT24 high Secretory Cells and Early Response Secretory Cells.

83. The method of claim 70, wherein the secretory cells express CXCL8.

84. The method of claim 70, wherein the goblet cells comprise one or both of: AZGP1 high Goblet Cells and SCGB1A1 high Goblet Cells.

85. The method of claim 70, where the ciliated cells comprise one or more upregulated genes selected from the group consisting of IFI27, IFIT1, IFI6, IFITM3, and GBP3.

86. The method of claim 70, wherein one or both of the ciliated cells and the goblet cells comprise increased gene expression of one or more IFN gene selected from any of Tables 2-4.

87. The method of claim 70, wherein ACE2 expression is upregulated compared to other epithelial cells among one or more of secretory cells, goblet cells, ciliated cells, developing ciliated cells, and deuterosomal cells.

88. The method of claim 70, wherein the mature ciliated cells are BEST4 high cilia high ciliated cells.

89. The method of claim 70, wherein the MHC-I and MHC-II genes comprise at least one or more of: HLA-A, HLA-C, HLA-F, HLA-E, HLA-DRB1, and HLA-DRA.

90. The method of claim 70, wherein the upregulated cholesterol and lipid biosynthesis genes comprise at least one or more of: FDFT1, MVK, FDPS, ACAT2, and HMGCS1.

91. The method of claim 69, wherein detecting one or more indicators is performed by using Simpson's index.

92. The method of claim 69, where a subject is determined to belong to the severe risk group if one or more of the following is detected in the sample:
a) proinflammatory cytokines comprising at least one or more of: IL1B, TNF, CXCL8, CCL2, CCL3, CXCL9, CXCL10, and CXCL11;
b) upregulation of alarmins comprising one or both of: S100A8 and S100A9;
c) 14%-26% of all epithelial cells are secretory cells;
d) elevated BPIFA1 high Secretory cells;
e) elevated KRT13 KRT24 high secretory cells;
f) macrophage population increase as compared to other immune cells;
g) upregulated genes in ciliated cells comprising one or both of: IL5RA and NLRP1;
h) no increase of at least one or more of: type I, type II, and type III interferon abundance;
i) elevated stress response factors comprising at least one or more of: HSPA8, HSPA1A, and DUSP1;
j) increased expression of one or more genes differentially expressed in COVID-19 WHO 6-8 according to Table 3 or Table 4;
k) reduced or absent antiviral/interferon response; and
l) reduced or absent mature ciliated cells.

93. The method of claim 92, wherein the macrophage population comprises at least one or more of: ITGAX High Macrophages, FFAR High Macrophages, Inflammatory Macrophages, and Interferon Responsive Macrophages.

94. The method of claim 69, where a subject is determined to belong to the mild/moderate risk group if one or more of the following is detected in the sample:
a) 4%-12% of all epithelial cells are Secretory Cells;
b) 10%-20% of all epithelial cells comprise Interferon Responsive Ciliated Cells;
c) upregulated ciliated cell genes comprising at least one or more of: IFI44L, STAT1, IFITM1, MX1, IFITM3, OAS1, OAS2, OAS3, STAT2, TAP1, HLA-C, ADAR, XAF1, IRF1, CTSS, and CTSB;
d) increase in type I interferon abundance;
e) high expression of interferon-responsive genes;
f) decreased expression of one or more genes differentially expressed in COVID-19 WHO 6-8 according to Table 3 or Table 4;
g) induction of type I interferon responses; and
h) high abundance of IFI6 and IFI27.

95. The method of claim 94, where the interferon-responsive genes comprise at least one or more of: STAT1, MX1, HLA-B, and HLA-C.

96. The method of claim 94, where the interferon response occurs in at least one or more of: MUC5AC high Goblet Cells, SCGB1A1 high Goblet Cells, Early Response Secretory Cells, Deuterosomal Cells, Interferon Responsive Ciliated Cells, and BEST4 high Cilia high Ciliated Cells.


SEQUENCE LISTING (19 sequences)

SEQ ID NO: 1 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 3 - protein, 7 aa, Simian virus 40
PKKKRKV

SEQ ID NO: 4 - protein, 10 aa, Simian virus 40
PKKKRKVEAS

SEQ ID NO: 5 - protein, 16 aa, Artificial Sequence
  Synthetic
KRPAATKKAGQAKKKK

SEQ ID NO: 6 - protein, 9 aa, Homo sapiens
PAAKRVKLD

SEQ ID NO: 7 - protein, 11 aa, Homo sapiens
RQRRNELKRSP

SEQ ID NO: 8 - protein, 38 aa, Homo sapiens
NQSSNFGPMKGGNFGGRSSGPYGGGGQYFAKPRNQGGY

SEQ ID NO: 9 - protein, 42 aa, Homo sapiens
RMRIZFKNKGKDTAELRRRRVEVSVELRKAKKDEQILKRRNV

SEQ ID NO: 10 - protein, 8 aa, Homo sapiens
VSRKRPRP

SEQ ID NO: 11 - protein, 8 aa, Homo sapiens
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis D virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK